(19) United States
(12) Patent Application Publication
(10) Pub. No.: US 2025/0332300 A1
(43) Pub. Date: Oct. 30, 2025

(54) HER2 TARGETING CYCLIC PEPTIDES AND CONJUGATES THEREOF

(71) Applicant: Novartis AG, Basel (CH)

(72) Inventors: Gonçalo dos Santos CLEMENTE, Basel (CH); Takeru EHARA, Tokyo (JP); Richard FELBER, Rheinfelden (DE); Matteo FISCHER, Basel (CH); Francois GAUTER, Rixheim (FR); Alexei KARPOV, Basel (CH); Marion LACAUD-BAUMLIN, Geispitzen (FR); Rafael BERMEO MALO, Basel (CH); Andreas MARZINZIK, Weil am Rhein (DE); Yoshihide MIZUKOSHI, Kanagawa (JP); Danielle PARK, Basel (CH); Markus Oliver RESCHKE, Birsfelden (CH); Sebastien RIPOCHE, Rantzwiller (FR); Michael SCHAEFER, Village Neuf (FR); Therese-Marie STACHYRA, Mulhouse (FR); Kyosuke UEDA, Yokohama-shi (JP); Patrik Peter ZUEGER, Basel (CH)

(21) Appl. No.: 19/184,167

(22) Filed: Apr. 21, 2025

Related U.S. Application Data
(60) Provisional application No. 63/637,572, filed on Apr. 23, 2024.
(60) Provisional application No. 63/760,270, filed on Feb. 19, 2025.

(51) Int. Cl. A61K 51/08; A61P 35/00; C07K 7/56

(52) U.S. Cl. CPC A61K 51/088; A61P 35/00; C07K 7/56; A61K 2121/00; A61K 2123/00

(57) ABSTRACT

Described herein are cyclic peptides targeting human epidermal growth factor receptor 2 (HER2), and their incorporation into compounds for radioligand imaging and therapies, as well as methods and/or uses of such compounds for the imaging, treatment and/or prevention of HER2-implicated diseases and disorders (e.g., cancer).

### CROSS-REFERENCE TO RELATED APPLICATIONS

This application claims priority to U.S. Provisional Application Ser. No. 63/637,572 filed Apr. 23, 2024 and U.S. Provisional Application Ser. No. 63/760,270 filed Feb. 19, 2025, which are hereby incorporated herein by reference in their entirety.

### SEQUENCE LISTING

The instant application contains a Sequence Listing which has been submitted herewith in ST.26 XML format and is hereby incorporated by reference in its entirety. Said ST.26 XML copy, created on Apr. 17, 2025, is named 763537_NIT-59597PC_ST26_SEQUENCE_LISTING and is 529.390 bytes in size.

### BACKGROUND

Since the discovery of the human epidermal growth factor receptor 2 (HER2) as an oncogenic driver in a subset of breast cancers and the development of HER2 targeted therapies, the prognosis of HER2 amplified breast cancer has significantly improved (Meric-Bernstram, F. et al., Clin Cancer Res. 25 (7): 2033-2041, 2019). Several monoclonal antibodies and tyrosine kinase inhibitors (TKIs) led to success in the HER2 amplified and/or overexpressed setting (Kunte et al., Cancer. 126 (19): 4278-4288 2020), which make up approximately 20% of all breast cancers. However, for the roughly 50% of breast cancers with low levels of HER2 expression and lack of amplification, development of these multiple classes of agents has proven unsuccessful (Tarantino, P. et al., J Clin Oncol. 38 (17): 1951-1962, 2020; Fehrenbacher, L. et al., J Clin Oncol. 38 (5): 444-453, 2020; Eiger, D. et al. Cancers (Basel). 13 (5): 1015, 2020). This is thought to be due to lack of antigen engagement in the case of the monoclonal antibodies and TKIs, dose-limiting toxicities caused by the payload itself, and/or on-target activity in normal tissues in the case of the antibody-drug conjugates. In addition to an overall lower antigen density in HER2-low tumors, a high degree of heterogeneity for HER2 expression between individual cells of a given lesion is widely accepted as another challenge (Ocaña, A. et al. Breast Cancer Res. 2020 Jan. 31; 22 (1): 15, Marchio, C. et al. Semin Cancer Biol. 2021 July; 72:123-135).

Several next generation HER2-directed antibody-drug conjugates are currently under clinical investigation (Kreutzfeldt et al. Am J Cancer Res. 2020 Apr. 1; 10 (4): 1045-1067). ENHERTU, an ADC coupling a topoisomerase I inhibitor to trastuzumab with a cleavable linker, has shown single agent antitumor activity in patients with HER low breast cancer. Currently, HER2 directed treatments in breast cancer are not indicated in patients with HER2 low disease. These patients do not benefit from targeted treatments and are usually treated with chemotherapy combinations. ENHERTU is currently being assessed in a randomized trial in HER2 low (defined as immunohistochemistry (IHC) 2+/in situ hybridization (ISH)− or IHC 1+ISH− or untested) versus investigator choice of chemotherapy (Keam, S J. Drugs. 2020 April; 80 (5): 501-508., Modi, S. et al. N Engl J Med. 2020 Feb. 13; 382 (7): 610-621). Further, low level and heterogeneous expression of HER2 has posed an obstacle to extending current HER2-directed treatments to more patients.

Accordingly, there is a need for targeting agents having both high and specific affinity for HER2 and radiotherapeutic conjugates thereof.

### SUMMARY

Provided herein are cyclic peptides that target human epidermal growth factor receptor 2 (HER2), compounds incorporating such cyclic peptides, which are suitable for radiolabeling, corresponding pharmaceutical compositions, and methods and/or uses of the HER2-targeting compounds (also referred to as HER2-targeting ligands) for the imaging and treatment of HER2-implicated cancers.

In particular, the present disclosure provides compounds, or pharmaceutically acceptable salts or solvates thereof, comprising:
a) at least one cyclized peptide that binds to human epidermal growth factor receptor 2 (HER2) with a dissociation constant (K_{D}) of about 1000 nM or less as measured by surface plasmon resonance (SPR) at a temperature of 25° C.; and
b) at least one imaging agent, chelating agent, radionuclide, or cytotoxic drug.

In an aspect, provided herein is a compound, or a pharmaceutically acceptable salt or solvate thereof, comprising:
a) at least one cyclized peptide \textcircled{P}, wherein \textcircled{P} is

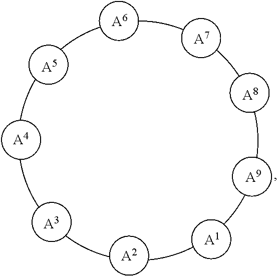

wherein A¹-A⁹ are as defined herein; and
b) at least one imaging agent, chelating agent, radionuclide, or cytotoxic drug, wherein the cyclized peptide \textcircled{P} is conjugated to the at least one imaging agent, chelating agent, radionuclide, or cytotoxic drug via any one of A¹-A⁹, optionally through a linker.

In some embodiments, the compounds are HER2-targeting compounds of formula (I), (Ia), (Ib), or (Ic):

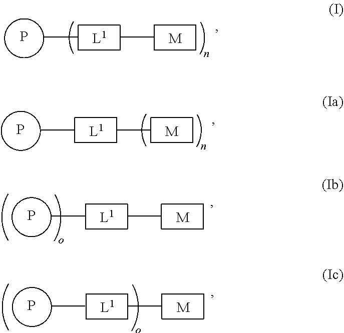

or a pharmaceutically acceptable salt or solvate thereof, wherein
P is a cyclic HER2-targeting peptide as defined above;
L¹ is, independently at each occurrence, a bond or a linker;
M is, independently at each occurrence, an imaging agent, a chelating agent, or a radionuclide,
wherein the chelating agent is optionally radiolabeled with a radionuclide;
n is 1, 2, 3, or 4; and
o is 1, 2, 3, or 4,
wherein any of P, L¹, or M is optionally substituted with an albumin binder, a biotin tag, or one or more polyethylene glycol (PEG) chains.

In certain embodiments, the HER2-targeting compounds are radiolabeled with a diagnostic or therapeutic radionuclide. Such radiolabeled compounds can be referred to a HER2-targeting radioligands, HER2-targeting radiotherapeutics, HER2-targeting radioimaging agents, or HER2-targeting radiopharmaceuticals.

The present disclosure further provides pharmaceutical compositions comprising the HER2-targeting compounds described herein, or a pharmaceutically acceptable salt or solvate thereof, and a pharmaceutically acceptable carrier.

The present disclosure further provides a combination comprising the HER2-targeting compounds described herein, or a pharmaceutically acceptable salt or solvate thereof, and one or more therapeutically active agents.

The present disclosure further provides a method of imaging HER2-related diseases and disorders, comprising administering to a subject in need thereof a diagnostically effective amount of a HER2-targeting ligand and/or a HER2-targeting radioligand, or a pharmaceutically acceptable salt or solvate thereof, or a pharmaceutical composition described herein.

The present disclosure further provides a method of treating and/or preventing HER2-related diseases and disorders, comprising administering to a subject in need thereof a therapeutically effective amount of a HER2-targeting ligand and/or a HER2-targeting radioligand, or a pharmaceutically acceptable salt or solvate thereof, or a pharmaceutical composition described herein.

### BRIEF DESCRIPTION OF THE DRAWINGS

FIG. 1 shows the results of the biodistribution studies performed with Example H1.

FIG. 2 shows the results of the biodistribution studies performed with Example H2.

FIG. 3 is a graph of the effects of administration of Example H1 and Example H2 on HCC1187 Breast Cancer CDX Tumor growth.

FIG. 4 is a graph of the effects of administration of Example H2 on ST313 Breast Cancer PDX Tumor growth.

FIG. 5 is a graph of the effects of administration of Example J1 and Example J2 on NSCLC PDX Tumor growth.

FIG. 6 is a graph of the effects of administration of Example J2 on ST3932 Breast Cancer PDX Tumor growth.

### DETAILED DESCRIPTION

Provided herein are cyclic peptides that target human epidermal growth factor receptor 2 (HER2), compounds incorporating such cyclic peptides, which are suitable for radiolabeling, corresponding pharmaceutical compositions, and methods and/or uses of the HER2-targeting compounds (also referred to as HER2-targeting ligands) for the imaging and treatment of HER2-implicated disease, such as, e.g., cancer.

Accordingly, described herein are HER2-targeting compounds (or alternatively, HER2-targeting ligands) comprising:
a) at least one cyclized peptide \textcircled{P}, wherein \textcircled{P} is

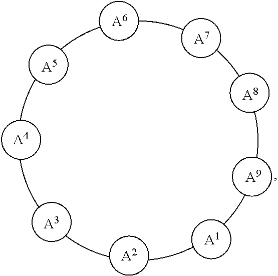

wherein A¹-A⁹ are as defined herein; and
b) at least one imaging agent, chelating agent, radionuclide, or cytotoxic drug,
wherein the cyclized peptide \textcircled{P} is conjugated to the at least one imaging agent, chelating agent, radionuclide, or cytotoxic drug via any one of A¹-A⁹, optionally through a linker.

The HER2-targeting compounds can be, inter alia, a compound of formula (I), (Ia), (Ib), (Ic), or (Id):

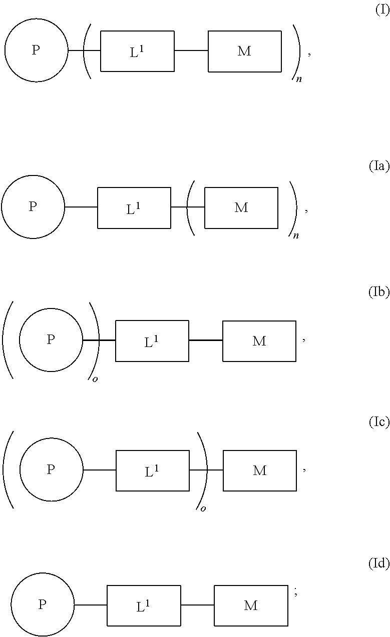

or a pharmaceutically acceptable salt or solvate thereof,
where P is a cyclic HER2-targeting peptide; M is, independently at each occurrence, selected from an imaging agent, a chelating agent optionally chelated to a radionuclide, a radionuclide, and a cytotoxic drug; L¹ is a bond or a linker adapted to form a chemical bond between P and M; and n and o are each independently 1, 2, 3, or 4.

The compounds disclosed herein, including the compounds of Formula (I), (Ia), (Ib), (Ic), or (Id), or a pharmaceutically acceptable salt or solvate thereof, exhibit strong binding to HER2, i.e., exhibit a dissociation constant (K_{D}) for human HER2 of about 1000 nM or less as measured by surface plasmon resonance (SPR) at a temperature of 25° C.

Compounds of Formula (I), (Ia), (Ib), (Ic), or (Id), or a pharmaceutically acceptable salt or solvate thereof, also exhibit prolonged tumor retention time.

Accordingly, described herein are methods of targeting HER2, imaging HER2-expression, and treating HER2-related disease, with a compound of any of Formulae (I), (Ia), (Ib), (Ic), or (Id) or a pharmaceutically acceptable salt or solvate thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

It is further appreciated that certain features of the disclosure, which are, for clarity, described in the context of separate embodiments, can also be provided in combination in a single embodiment. Conversely, various features of the invention, which are, for brevity, described in the context of a single embodiment, can also be provided separately or in any suitable sub-combination.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

The terms “peptide,” “polypeptide,” and “protein” are used interchangeably herein and typically refer to a molecule comprising a chain of two or more amino acids (e.g., L-amino acids, D-amino acids, modified or derivatized amino acids, amino acid analogs, amino acid mimetics, etc.).

Unless otherwise indicated, naturally occurring L-amino acids and D-amino acids are both represented by either conventional three-letter, or capitalized one-letter, amino acid designations of Table 1. In some embodiments, naturally occurring L-amino acids are represented by either conventional three-letter, or capitalized one-letter, amino acid designations of Table 1. In some embodiments, D-amino acids, are represented by lower-case one-letter amino acid designations corresponding to one-letter designations of Table 1, i.e., g, a, l, m, f, w, k, q, e, s, p, v, i, c, y, h, r, n, d, and t.

TABLE 1
Naturally occurring amino acids

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

The term “L-amino acid,” as used herein, refers to the “L” isomeric form of an amino acid, and conversely the term “D-amino acid” refers to the “D” isomeric form of an amino acid (e.g., (D) Asp or D-Asp; (D) Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, D-arginine can be represented as “arg” or “r.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g., sarcosine, ornithine, etc.), frequently employed three- or four-character codes are employed for residues thereof, including, Beta-Ala, b-Ala, or bA (β-alanine), Sar or Sarc (sarcosine, i.e., N-methylglycine), Aib (α-aminoisobutyric acid), Dab (2,4-diaminobutanoic acid), Dap (2,3-diaminopropanoic acid), γ-Glu (γ-glutamic acid), Gaba (γ-aminobutanoic acid), β-Pro (pyrrolidine-3-carboxylic acid), and Abu (2-aminobutyric acid).

Further, non-limiting examples of non-naturally occurring amino acids that may appear, e.g., in the compounds disclosed herein including those of Formulae (I), (Ia), (Ib), (Ic) or (Id) appear in Table 2 below.

TABLE 2
Non-limiting list of non-naturally occurring amino acids that may be incorporated
into the HER2-targeting cyclic peptides of the present disclosure.

| | | Examples of incorporation |
|---|---|---|
| AA abbreviation | AA structure | into a cyclic peptide |
| AlloT | 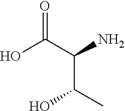 | 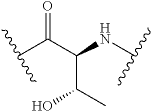 [SMILES: *N[C@H](C(*)=O)[C@H](C)O] |
| H-bA(2S-Me) | 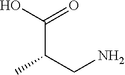 | 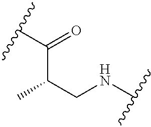 [SMILES: *NC[C@H](C)C(*)=O] |
| H-hCys | 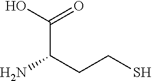 | 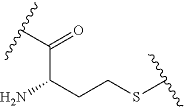 [SMILES: *SCC[C@H](N)C(*)=O] |
| 3-(CH2NH2)Bz | 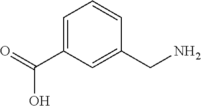 [SMILES: NCc1cccc(C(=O)O)c1] | 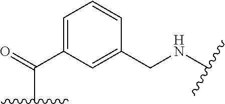 [SMILES: *NCc1cccc(C(*)=O)c1] |
| H-F(3aa) | 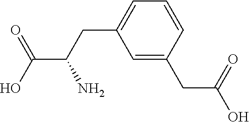 [SMILES: N[C@@H](Cc1cccc(CC(=O)O)c1)C(=O)O] | 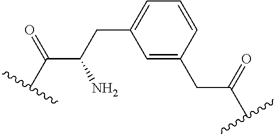 [SMILES: *C(=O)Cc1cccc(C[C@H](N)C(*)=O)c1] |
| HAsp | 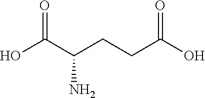 | 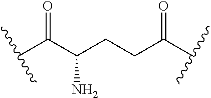 [SMILES: *C(=O)CC[C@H](N)C(*)=O] |
| NAc-Asp | 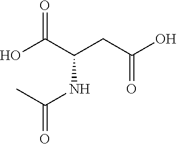 | 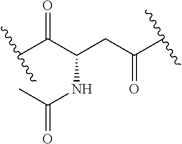 [SMILES: *C(=O)C[C@H](NC(C)=O)C(*)=O] |
| NAc-E | 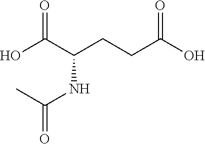 | 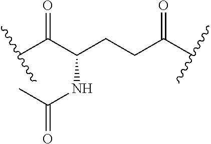 [SMILES: *C(=O)CC[C@H](NC(C)=O)C(*)=O] |
| hE | 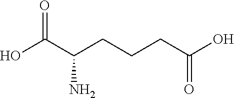 [SMILES: N[C@@H](CCCC(=O)O)C(=O)O] | 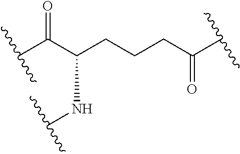 [SMILES: *N[C@@H](CCCC(*)=O)C(*)=O] |
| | | or |
| | | 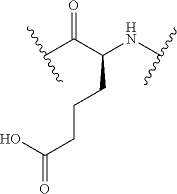 |
| Malonyl-hE | 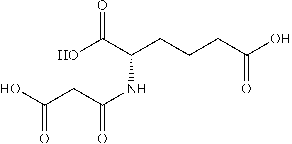 | 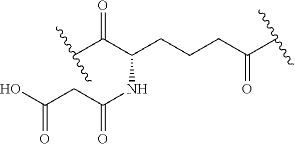 |
| Succinyl-hE | 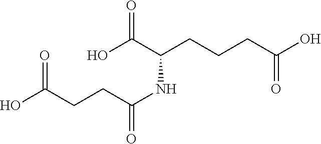 [SMILES: O=C(O)CCC[C@H](NC(=O)CCC(=O)O)C(=O)O] | 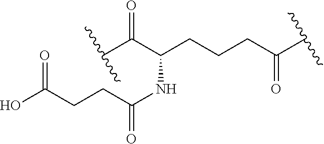 [SMILES: *C(=O)CCC[C@H](NC(=O)CCC(=O)O)C(*)=O] |
| COCH2Tet (1H)-hE | 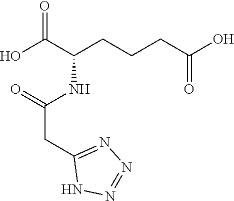 [SMILES: O=C(O)CCC[C@H](NC(=O)Cc1nnn[nH]1)C(=O)O] | 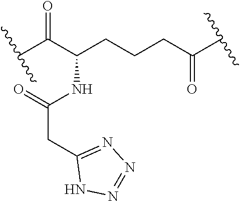 [SMILES: *C(=O)CCC[C@H](NC(=O)Cc1nnn[nH]1)C(*)=O] |
| NAc-hE | 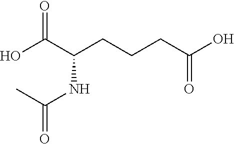 | 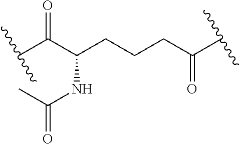 [SMILES: *C(=O)CCC[C@H](NC(C)=O)C(*)=O] |
| HOAc-hE | 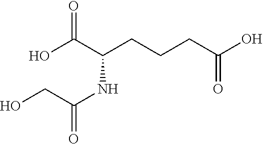 [SMILES: O=C(O)CCC[C@H](NC(=O)CO)C(=O)O] | 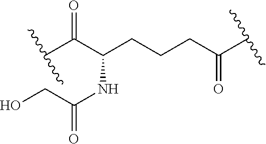 [SMILES: *C(=O)CCC[C@H](NC(=O)CO)C(*)=O] |
| H-Dap | 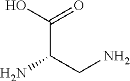 | 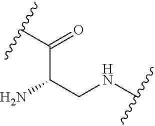 [SMILES: *NC[C@H](N)C(*)=O] |
| H-bA(2S-OH) | 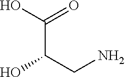 | 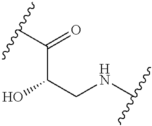 [SMILES: *NC[C@H](O)C(*)=O] |
| beta2E | 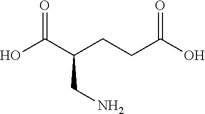 [SMILES: NC[C@H](CCC(=O)O)C(=O)O] | 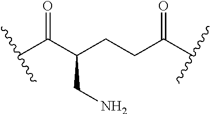 [SMILES: *C(=O)CC[C@@H](CN)C(*)=O] |
| H-Alk1 | 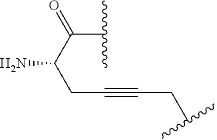 [SMILES: *CC#CC[C@H](N)C(*)=O] | 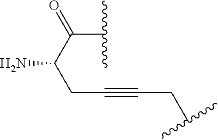 [SMILES: *CC#CC[C@H](N)C(*)=O] |
| H-hE | 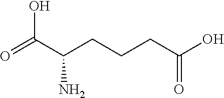 [SMILES: N[C@@H](CCCC(=O)O)C(=O)O] | 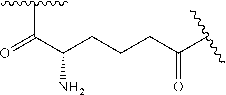 [SMILES: *C(=O)CCC[C@H](N)C(*)=O] |
| NAc-S(aa) | 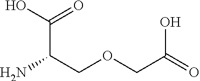 [SMILES: N[C@@H](COCC(=O)O)C(=O)O] | 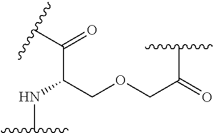 [SMILES: *N[C@@H](COCC(*)=O)C(*)=O] |
| 3PyA(6OH) | 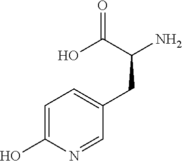 [SMILES: N[C@@H](Cc1ccc(O)nc1)C(=O)O] | 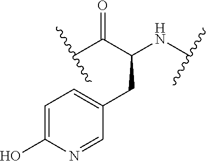 [SMILES: *N[C@@H](Cc1ccc(O)nc1)C(*)=O] |
| 4PyA | 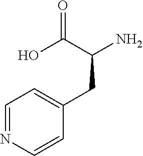 [SMILES: N[C@@H](Cc1ccncc1)C(=O)O] | 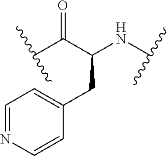 [SMILES: *N[C@@H](Cc1ccncc1)C(*)=O] |
| NMeS | 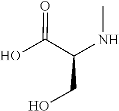 | 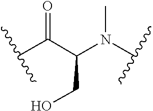 [SMILES: *C(=O)[C@H](CO)N(*)C] |
| Dap | 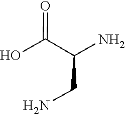 | 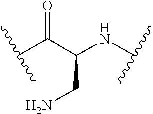 [SMILES: *N[C@@H](CN)C(*)=O] |
| | | or |
| | | 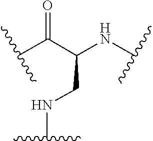 [SMILES: *NC[C@H](N*)C(*)=O] |
| KCOpipzaa | 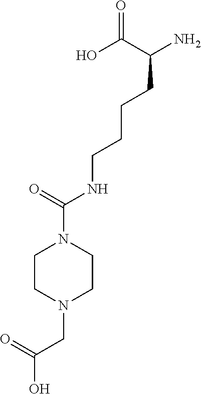 [SMILES: N[C@@H](CCCCNC(=O)N1CCN(CC(=O)O)CC1)C(=O)O] | 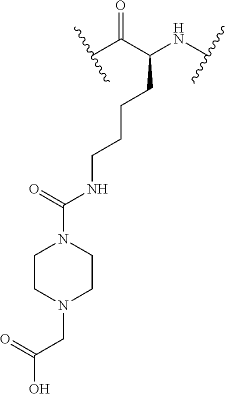 [SMILES: *N[C@@H](CCCCNC(=O)N1CCN(CC(=O)O)CC1)C(*)=O] |
| Lys(Alloc) | 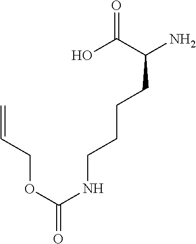 [SMILES: C=CCOC(=O)NCCCC[C@H](N)C(=O)O] | 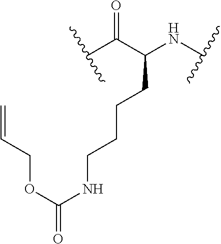 [SMILES: *N[C@@H](CCCCNC(=O)OCC=C)C(*)=O] |
| propargyl-Gly | 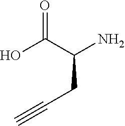 | 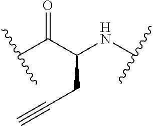 [SMILES: *N[C@@H](CC#C)C(*)=O] |
| Q(Mann) | 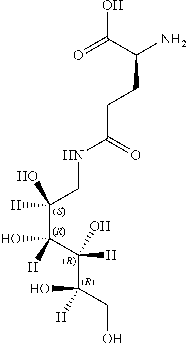 | 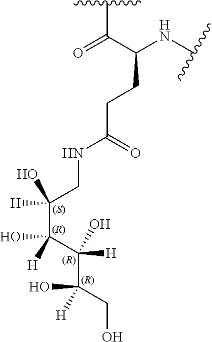 |
| SAR14 | 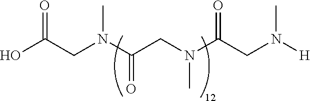 | 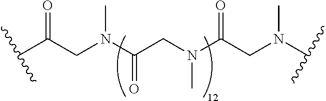 |
| NMehF | 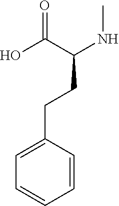 | 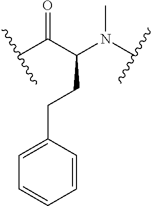 [SMILES: *C(=O)[C@H](CCc1ccccc1)N(*)C] |
| NMehF(3Cl) | 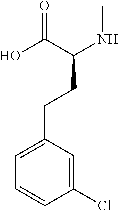 | 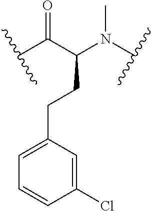 [SMILES: *C(=O)[C@H](CCc1cccc(Cl)c1)N(*)C] |
| Ahp | 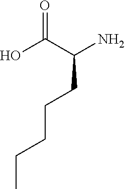 | 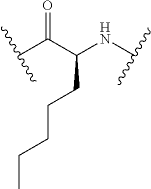 |
| NMeAhp | 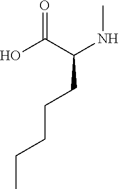 | 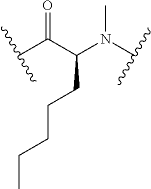 |
| Aoc | 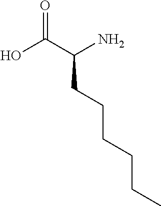 | 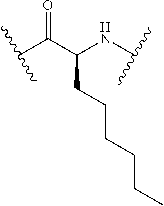 |
| NMeAoc | 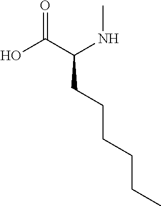 | 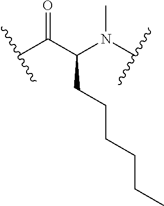 |
| NMeh2PyA | 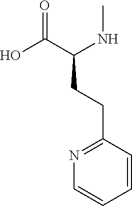 | 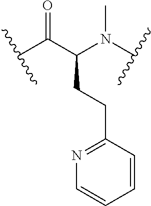 [SMILES: *C(=O)[C@H](CCc1ccccn1)N(*)C] |
| NMe4PyA | 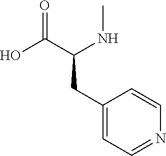 | 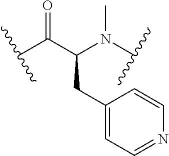 [SMILES: *C(=O)[C@H](Cc1ccncc1)N(*)C] |
| NMecHexA | 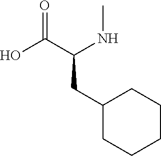 | 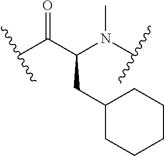 [SMILES: *C(=O)[C@H](CC1CCCCC1)N(*)C] |
| NMeh3PyA | 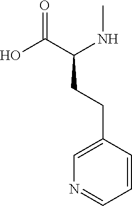 | 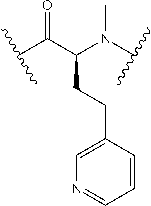 [SMILES: *C(=O)[C@H](CCc1cccnc1)N(*)C] |
| NMehF(3OH)) | 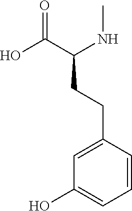 | 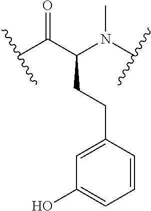 [SMILES: *C(=O)[C@H](CCc1cccc(O)c1)N(*)C] |
| NMehF(4Cl) | 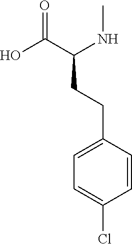 | 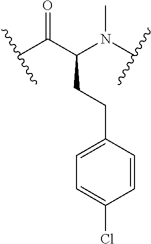 [SMILES: *C(=O)[C@H](CCc1ccc(Cl)cc1)N(*)C] |
| NMehF(4F) | 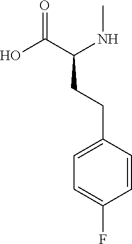 | 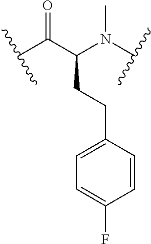 [SMILES: *C(=O)[C@H](CCc1ccc(F)cc1)N(*)C] |
| NMehY | 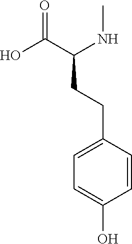 | 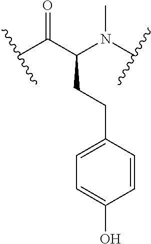 [SMILES: *C(=O)[C@H](CCc1ccc(O)cc1)N(*)C] |
| NMeNle | 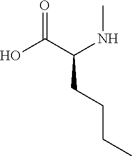 | 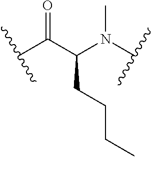 |
| NMeS(Ph) | 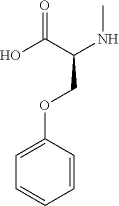 | 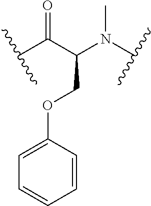 [SMILES: *C(=O)[C@H](COc1ccccc1)N(*)C] |
| W(5OH) | 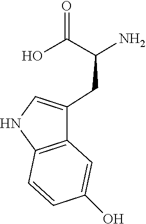 [SMILES: N[C@@H](Cc1c[nH]c2ccc(O)cc12)C(=O)O] | 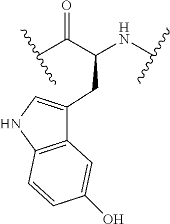 [SMILES: *N[C@@H](Cc1c[nH]c2ccc(O)cc12)C(*)=O] |
| W(5OAc) | 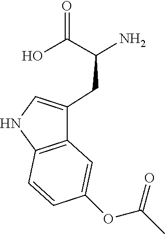 [SMILES: CC(=O)Oc1ccc2[nH]cc(C[C@H](N)C(=O)O)c2c1] | 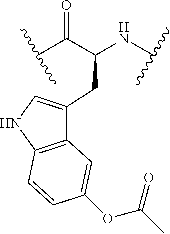 [SMILES: *N[C@@H](Cc1c[nH]c2ccc(OC(C)=O)cc12)C(*)=O] |
| W8N | 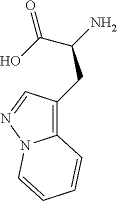 [SMILES: N[C@@H](Cc1cnn2ccccc12)C(=O)O] | 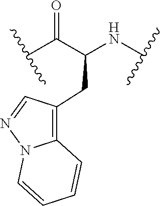 [SMILES: *N[C@@H](Cc1cnn2ccccc12)C(*)=O] |
| W(6OH) | 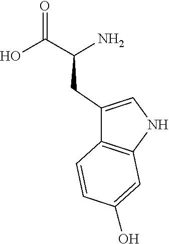 [SMILES: N[C@@H](Cc1c[nH]c2cc(O)ccc12)C(=O)O] | 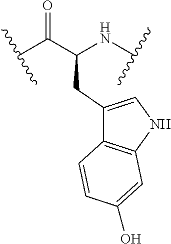 [SMILES: *N[C@@H](Cc1c[nH]c2cc(O)ccc12)C(*)=O] |
| W5F | 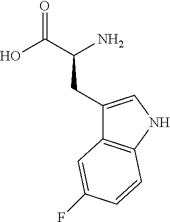 [SMILES: N[C@@H](Cc1c[nH]c2ccc(F)cc12)C(=O)O] | 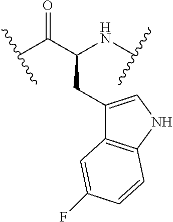 [SMILES: *N[C@@H](Cc1c[nH]c2ccc(F)cc12)C(*)=O] |
| W(2Me) | 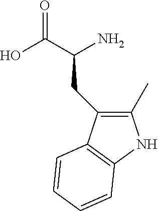 [SMILES: Cc1[nH]c2ccccc2c1C[C@H](N)C(=O)O] | 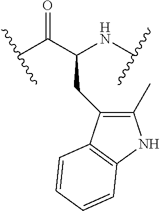 [SMILES: *N[C@@H](Cc1c(C)[nH]c2ccccc12)C(*)=O] |
| IndaA | 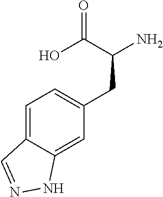 [SMILES: N[C@@H](Cc1ccc2cn[nH]c2c1)C(=O)O] | 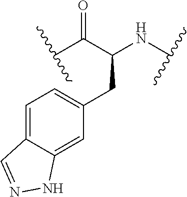 [SMILES: *N[C@@H](Cc1ccc2cn[nH]c2c1)C(*)=O] |
| CproG | 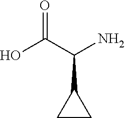 | 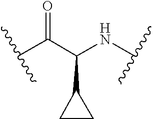 [SMILES: *N[C@H](C(*)=O)C1CC1] |
| CBuG | 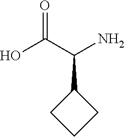 | 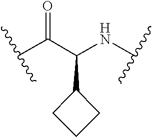 [SMILES: *N[C@H](C(*)=O)C1CCC1] |
| NMeDap | 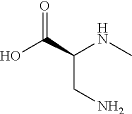 | 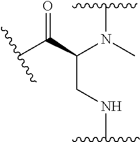 [SMILES: *NC[C@@H](C(*)=O)N(*)C] |
| NMeC or NMeCys | 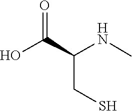 | 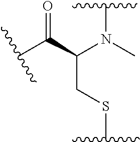 [SMILES: *SC[C@@H](C(*)=O)N(*)C] |
| NMeE | 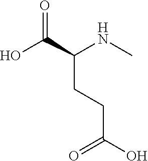 | 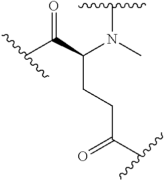 [SMILES: *C(=O)CC[C@@H](C(*)=O)N(*)C] |
| NMehCys or NMehC | 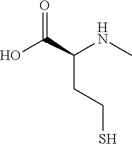 | 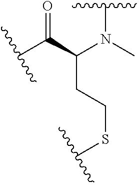 [SMILES: *SCC[C@@H](C(*)=O)N(*)C] |
| NMeD | 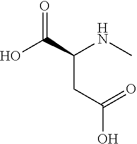 | 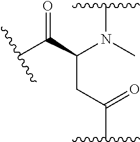 [SMILES: *C(=O)C[C@@H](C(*)=O)N(*)C] |
| NMeDab | 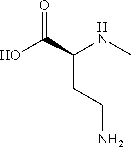 | 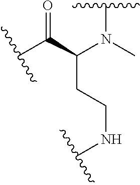 [SMILES: *NCC[C@@H](C(*)=O)N(*)C] |
| NMe-K(TFA) | 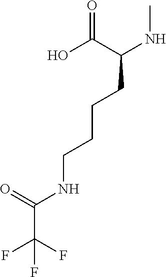 [SMILES: CN[C@@H](CCCCNC(=O)C(F)(F)F)C(=O)O] | 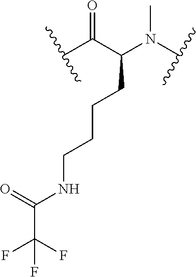 [SMILES: *C(=O)[C@H](CCCCNC(=O)C(F)(F)F)N(*)C] |
| NMeF(4F) | 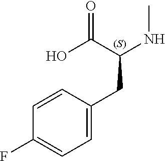 | 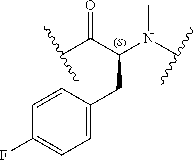 |
| MeKAc | 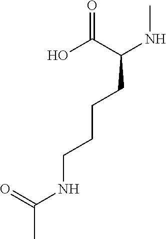 | 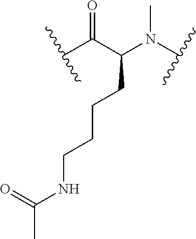 [SMILES: *C(=O)[C@H](CCCCNC(C)=O)N(*)C] |
| MeK (COCH2OH) | 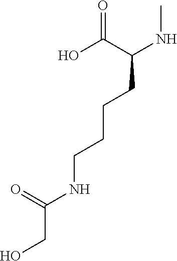 | 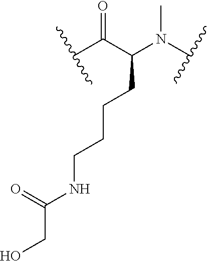 [SMILES: *C(=O)[C@H](CCCCNC(=O)CO)N(*)C] |
| MeK (COEtOH) | 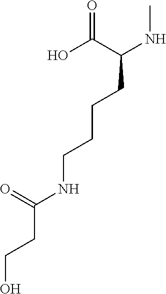 [SMILES: CN[C@@H](CCCCNC(=O)CCO)C(=O)O] | 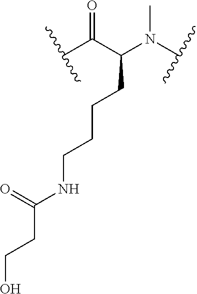 [SMILES: *C(=O)[C@H](CCCCNC(=O)CCO)N(*)C] |
| K(N3) | 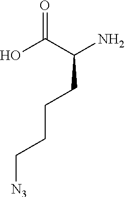 [SMILES: [N-]=[N+]=NCCCC[C@H](N)C(=O)O] | 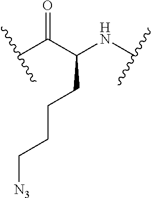 |
| Orn | 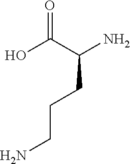 | 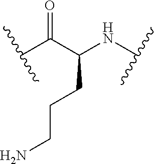 [SMILES: *N[C@@H](CCCN)C(*)=O] |
| hK | 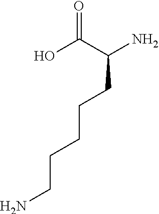 [SMILES: NCCCCC[C@H](N)C(=O)O] | 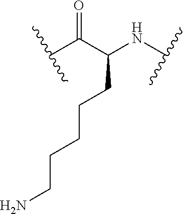 |
| MeK (Malonyl) | 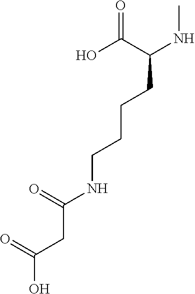 [SMILES: CN[C@@H](CCCCNC(=O)CC(=O)O)C(=O)O] | 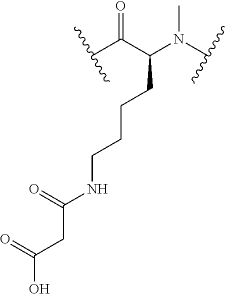 [SMILES: *C(=O)[C@H](CCCCNC(=O)CC(=O)O)N(*)C] |
| b-A, b-Ala, or Beta-Ala | 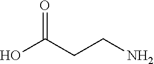 | 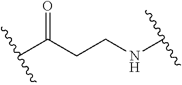 [SMILES: *NCCC(*)=O] |

Amino acids of the D-isomeric form may be located at any of the positions in the HER2-targeting compounds disclosed herein (e.g., any of A¹-A¹⁰ appearing in the compounds of Formulae (I), (Ia), (Ib), (Ic), (Id), or (I-i)).

Peptides may be naturally occurring, synthetically produced, or recombinantly expressed. Peptides may also comprise additional groups modifying the amino acid chain, for example, functional groups added via post-modification. Examples of post-modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. The term peptide also includes modifications, such as, but not limited to, those described above, of amino acids falling between the amino and carboxy termini.

The skilled artisan will recognize that the peptide sequences disclosed herein in some cases are depicted having the left end of the sequence being the N-terminus of the peptide and the right end of the sequence being the C-terminus of the peptide, or in other cases are depicted having the left end of the sequence being the C-terminus of the peptide and the right end of the sequence being the N-terminus of the peptide. The context of the use of the sequence will make such directionality clear. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the disclosure, a C-terminal “—OH” moiety may be substituted for a C-terminal “—NH₂” moiety, and vice-versa.

The phrase “amino acid,” “amino acid residue,” or “residue” as used herein refers to an amino acid, a modified or derivatized amino acid, an amino acid analog, or an amino acid mimetic that is incorporated into a peptide by an amide bond or an amide bond mimetic.

Unless indicated otherwise the names of naturally occurring and non-naturally occurring amino acid residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14 (2), (1975). To the extent that the names and abbreviations of amino acids and aminoacyl residues employed in this specification and appended claims differ from those suggestions, they will be made clear to the reader.

One of skill in the art will appreciate that certain amino acids and other chemical moieties are modified when bound to another molecule. For example, an amino acid side chain may be modified when it is derivatized (e.g., with a substituent) or forms an intramolecular bridge with another amino acid side chain, e.g., one or more hydrogens may be removed or replaced by the bond.

In some embodiments, amino acid residues in the disclosed cyclic peptides may exist in the zwitterionic form. As will be appreciated by one of skill in the art, a zwitterion is a molecule that contains both a positive charge and a negative charge, resulting in an overall neutral charge. For example, an amino acid in the zwitterion form includes a carboxylate ion (negative charge) and an ammonium ion (positive charge). Zwitterionic amino acids may exist at neutral pH.

As used herein, “about” means within +10% of a value.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” means physiologically and pharmaceutically acceptable salts of the compounds disclosed herein, i.e., salts that retain the desired biological activity of the compounds and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of the compounds disclosed herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

As used herein, the term “solvate” means a physical association of a compound disclosed herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolable solvates. In general, such solvents selected for the purpose of the disclosure do not interfere with the biological activity of the solute. Non-limiting examples of suitable solvates include hydrates, ethanolates, methanolates, and the like.

As used herein, the term “hydrate” means a solvate wherein the solvent molecule(s) is/are water. In an embodiment, the solvate is a hydrate.

As used herein, the term “stereoisomer” means a molecule that has the same molecular formula and sequence of bonded atoms but differs in the three-dimensional orientations of its atoms in space.

The term “tautomer” means two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

As used herein, the term “treat,” “treatment,” or “treating” means decrease, suppress, attenuate, diminish, arrest, or stabilize the development or progression of a disorder or disease.

As used herein, the term “prevent” or “prevention” means no disorder or disease development if none had occurred, or no further disorder or disease development if there had already been development of the disorder or disease. Also contemplated is the ability to prevent some or all of the symptoms associated with the disorder or disease.

The term “pharmaceutical composition” is defined herein to refer to a mixture (e.g., a solution or an emulsion) containing at least one active ingredient or therapeutic agent to be administered to a subject, e.g., a human, in order to prevent or treat a particular disease or condition affecting the subject.

The term “a therapeutically effective amount” of a compound (i.e., a compound of Formula (I), or a pharmaceutically acceptable salt thereof) of the present disclosure refers to an amount of the compound of the present disclosure that will elicit the biological or medical response of a subject (patient or subject), for example, reduction or inhibition of enzymatic, protein, or cellular activity, or ameliorate symptoms, alleviate conditions, slow or delay disease progression. The therapeutically effective dosage of a compound, the pharmaceutical composition, or the combinations thereof, can be dependent on the body weight, age, sex, and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The term “detectably effective amount” of a compound (i.e., a compound of Formula (I) or a pharmaceutically acceptable salt thereof) as used herein refers to an amount of the compound of the present disclosure sufficient to provide an acceptable image using equipment that is available in a clinical setting.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. A prefix such as “Cᵤ₋ᵥ” or (Cᵤ-Cᵥ) indicates that the following group has from u to v carbon atoms. For example, “C₁₋₆-alkyl” and “C₁-C₆ alkyl” both indicate that the alkyl group has from 1 to 6 carbon atoms.

The term “alkyl” is a straight or branched saturated hydrocarbon. For example, an alkyl group can have 1 to 10 carbon atoms (i.e., C₁₋₁₀-alkyl), 1 to 8 carbon atoms (i.e., C₁₋₈-alkyl), 1 to 5 carbon atoms (i.e., C₁₋₅-alkyl), 1 to 4 carbon atoms (i.e., C₁₋₄-alkyl), or 1 to 3 carbon atoms (i.e., C₁₋₃-alkyl). Examples of alkyl groups include, but are not limited to, methyl(Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃) CH₂CH₃), tert-butyl (1-bu, 1-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃) CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₁₋₆-alkyl. In another embodiment, alkyl refers to C₁₋₄alkyl. In another embodiment, alkyl refers to C₁₋₃alkyl.

The term “alkylene” refers to a bivalent alkyl group. For example, an alkylene group can have 1 to 10 carbon atoms (i.e., (C₁₋₁₀alkylene), 1 to 5 carbon atoms (i.e., (C₁₋₅alkylene), 1 to 2 carbon atoms (i.e., (C₁₋₂alkylene), or 1 carbon atom (i.e., C₁-alkylene). Examples of alkylene groups include, but are not limited to, methylene (—CH₂—), ethylene (—CH₂CH₂—), n-propylene (—CH₂CH₂CH₂—), n-butylene (—CH₂CH₂CH₂CH₂—), etc.

The term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, biphenyl, and naphthyl. In some embodiments, aryl groups have from six to sixteen carbon atoms (e.g., C₆₋₁₆-aryl). In some embodiments, aryl groups have from six to twelve carbon atoms (e.g., C₆₋₁₂-aryl). In some embodiments, aryl groups have six carbon atoms (e.g., C₆-aryl, which may also be referred to as phenyl).

The term “halo” or “halogen” refers to bromo (—Br), chloro (—Cl), fluoro (—F), or iodo (—I). In an embodiment, halo refers to fluoro.

The term “haloalkyl” refers to a straight- or branched-chain alkyl group having from 1 to 12 carbon atoms, 1 to 6 carbon atoms, 1 to 4 carbon atoms, or 1 to 3 carbon atoms in the chain optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples. In an embodiment, haloalkyl refers to C₍₁₋₆₎ haloalkyl. In another embodiment, haloalkyl refers to C₍₁₋₄₎ haloalkyl. In another embodiment, alkyl refers to C₍₁₋₃₎ haloalkyl.

The term “cycloalkyl” refers to a saturated or partially unsaturated all carbon ring system having 3 to 8 carbon atoms (i.e., C₃₋₈cycloalkyl), or 3 to 6 carbon atoms (i.e., C₃₋₆cycloalkyl), wherein the cycloalkyl ring system has a single ring or multiple rings, e.g., in a spirocyclic or bicyclic form. Exemplary cycloalkyls include, but are not limited to, cyclopropyl, cyclobutyl, cyclopentyl, cyclohexyl, cycloheptyl, and cyclooctyl. Some cycloalkyl groups may exist as spirocycloalkyls, wherein two cycloalkyl rings are fused through a single carbon atom; for example and without limitation, an example of a spiropentyl group is

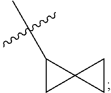 [SMILES: *C1CC12CC2]

for example and without limitation, examples of spirohexyl groups include

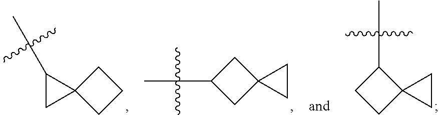 [SMILES: *C1CC12CCC2 ; *C1CC2(CC2)C1 ; *C1CCC12CC2]

for example and without limitation examples of cycloheptyl groups include

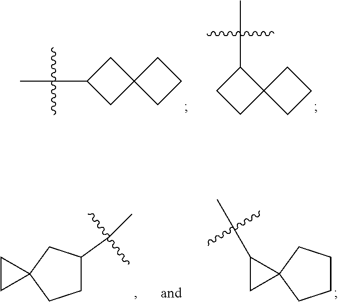

for example and without limitation examples of cyclooctyl groups include

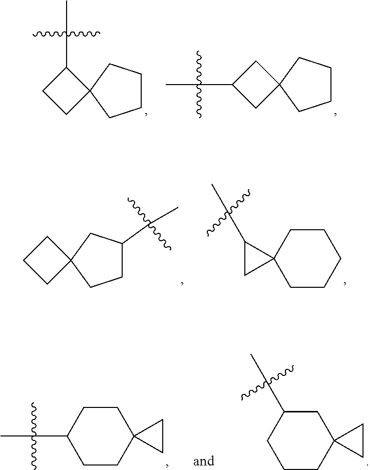 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Bicyclic cycloalkyl ring systems also include

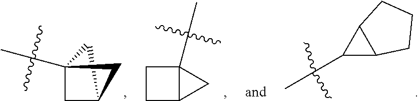

The terms “heterocycle,” “heterocyclyl,” and “heterocycloalkyl” each refer to a saturated or partially unsaturated ring system that has at least one atom other than carbon in the ring system, wherein the atom is selected from the group consisting of oxygen, nitrogen and sulfur. The heterocyclyl group may, for example, consist of a single ring or multiple rings (e.g., in the form of a spirocyclic or bicyclic ring system). Exemplary heterocycles include, but are not limited to oxetanyl, aziridinyl, azetidinyl, pyrrolidinyl, piperidinyl, piperazinyl, morpholinyl, tetrahydropyranyl, tetrahydrofuranyl, and thiomorpholinyl.

As used herein, the term “heteroaryl” or “heteroaromatic,” employed alone or in combination with other terms, refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. Alternatively, heteroaryl substituents may be defined by the number of atoms in the heteroaryl core, e.g., 5-6 membered heteroaryl indicates the number of carbon and heteroatoms contained in the heteroaryl core. As used herein, heteroaryl includes polycyclic ring systems wherein at least one ring has aromatic character and thus, may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

When two terms are combined, such as, for example, “alkylaryl” and “alkylheteroaryl,” it is intended that the elements defined by those terms are covalently attached, and that such covalent attachment may occur at any site of each element. For example, C₁₋₆-alkyl (5 to 6 membered heteroaryl) indicates a C₁₋₆-alkyl that is covalently attached to a 5 to 6 membered heteroaryl. In some embodiments, the 5 to 6 membered heteroaryl may be covalently attached to the C₁₋₆-alkyl at the C₁ or C₆ carbon, thereby providing a linear alkylheteroaryl group. In some embodiments, the 5 to 6 membered heteroatom is covalently attached to the C₁₋₆-alkyl at any one of C₂, C₃, C₄, and C₅ carbon atoms, thereby providing a branched alkylheteroaryl group. Likewise, the C₁₋₆-alkyl may be attached to any atom of the 5 to 6 membered heteroaryl. Further, a substituent defined using combined terms, e.g., “alkylaryl” and “alkylheteroaryl,” may be connected to the compound via any atom of either of the elements defined by the combined terms. For example, an “alkylaryl” or “alkylheteroaryl” substituent may be connected to the compound via the alkyl group or the aryl/heteroaryl group. In various embodiments, an “alkylaryl” or “alkylheteroaryl” substituent is connected to the compound via the alkyl group. In various other embodiments, the “alkylaryl” or “alkylheteroaryl” substituent is connected to the compound via the aryl or heteroaryl group.

The term “independently selected” is used herein to indicate that, for a variable which occurs in more than one location in a genus, the identity of the variable is determined separately in each instance. For example, if R^{x} appears as a substituent on two different atoms, the two instances of R^{x} may be the same moiety, or different moieties. The same is true if a single atom is substituted with more than one instance of R^{x}. The identity of R$ in each instance is determined independently of the identity of the other(s).

In non-limiting embodiments, when a substituent is designated in the form:

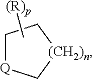

then it is to be understood that substituent R can occur p number of times on the ring, and R can be a different moiety at each occurrence. It is understood that each R may replace any hydrogen atom attached to a ring atom, including one or both of the (CH₂) n hydrogen atoms. Further, in the above example, should the variable Q be defined to include hydrogen atoms, such as when Q is CH₂, NH, etc., any floating substituent such as R in the above example, can replace a hydrogen atom of the Q variable as well as a hydrogen atom in any other non-variable component of the ring.

The terms “connected to” and “conjugated to” as used herein may be used interchangeably and are meant to indicate that two independent constituents are joined together such as by one or more covalent bonds. In some embodiments, the cyclized peptide is conjugated to or connected to a chelating agent via a covalent bond.

The terms “chelated to” and “complexed to” as used herein may be used interchangeably and are meant to indicate that two independent constituents are joined together such as by one or more non-covalent bonds, e.g., coordination bonds.

The term “radiolabeled” as used herein means that a non-radioactive compound is labeled with a radioisotope. Radiolabeling can be achieved, e.g., via chelation or complexation of a chelator with an appropriate radionuclide. Radiolabeling can also refer to chemically substituting one group on a compound for a radionuclide, such as, e.g., in the case of ¹⁸F.

Furthermore, it is intended that within the scope of the present invention, any element, in particular when mentioned in relation to a peptide of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎ alkyl includes not only —CH₃, but also CD₃; not only CH₂CH₃, but also CD₂CD₃, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, stabilizers (i.e., a pharmaceutically acceptable stabilizer), binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the imaging, therapeutic, or pharmaceutical compositions is contemplated.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### HER2-Targeting Compounds

The present disclosure provides HER2-targeting compounds, or pharmaceutically acceptable salts or solvates thereof, comprising:
a) at least one cyclized peptide that binds to human epidermal growth factor receptor 2 (HER2) with a dissociation constant (K_{D}) of about 1000 nM or less as measured by surface plasmon resonance (SPR) at a temperature of 25° C.; and
b) at least one imaging agent, chelating agent, radionuclide, or cytotoxic drug.

In an embodiment, the K_{D} is about 200 nM or less.

In another embodiment, the cyclized peptide comprises seven to fifteen amino acids and/or amino acid derivatives.

In yet another embodiment, the cyclized peptide comprises at least two consecutive amino acids or amino acid derivatives independently selected from tryptophan and tryptophan derivatives.

In an aspect, provided herein is a compound, or a pharmaceutically acceptable salt or solvate thereof, comprising:

a) at least one cyclized peptide \textcircled{P}, wherein \textcircled{P} is

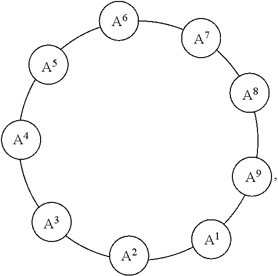

wherein A¹-A⁹ are as defined herein; and
b) at least one imaging agent, chelating agent, radionuclide, or cytotoxic drug, wherein at least one cyclized peptide \textcircled{P} is conjugated to the at least one imaging agent, chelating agent, radionuclide, or cytotoxic drug via any one of A¹-A⁹, optionally through a linker.

In some embodiments, the HER2-targeting compound is a compound of formula (I), (Ia), (Ib), or (Ic):

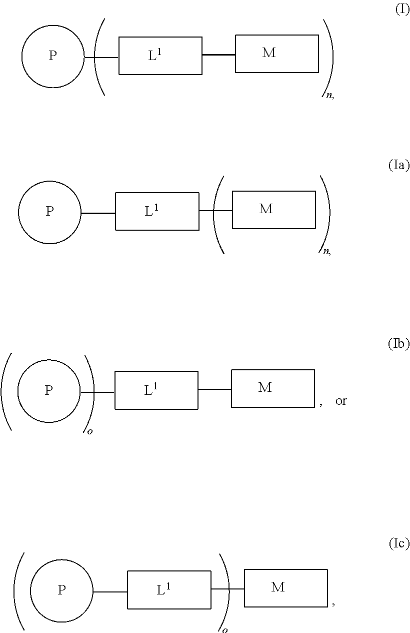

or a pharmaceutically acceptable salt or solvate thereof,
   wherein:
P is a cyclic HER2-targeting peptide as defined above;
L¹ is, independently at each occurrence, a bond or a linker;
M is, independently at each occurrence, an imaging agent, a chelating agent, or a radionuclide,
wherein the chelating agent is optionally radiolabeled with a radionuclide;
n is 1, 2, 3, or 4; and
is 1, 2, 3, or 4,
wherein any of P, L¹, or M are optionally substituted with an albumin binder, a biotin tag, or one or more polyethylene glycol (PEG) chains.

In some embodiments, L¹ is a bond. In some embodiments, L¹ is a linker.

In some embodiments of the compounds of compound formula (I), (Ia), (Ib), (Ic), or (Id), or a pharmaceutically acceptable salt or solvate thereof, M is, independently at each occurrence, an imaging agent, a chelating agent, or a radionuclide, wherein the chelating agent is optionally radiolabeled with a radionuclide. In further embodiments, at least one M is a chelating agent, wherein the chelating agent is radiolabeled with a radionuclide. In further embodiments, at least one M is an imaging agent. In some embodiments, at least one M is a radionuclide.

In some embodiments of the compounds of compound formula (I), (Ia), (Ib), (Ic), or (Id), or a pharmaceutically acceptable salt or solvate thereof, n is 1, 2, 3, or 4. In further embodiments, n is 1, 2, or 3. In further embodiments, n is 1 or 2. In further embodiments, n is 1. In further embodiments, n is 2. In further embodiments, n is 3. In further embodiments, n is 4.

In some embodiments of the compound of formula (I), (Ia), (Ib), (Ic), or (Id), or a pharmaceutically acceptable salt or solvate thereof, o is 1, 2, 3, or 4. In further embodiments, o is 1, 2, or 3. In further embodiments, o is 1 or 2. In further embodiments, o is 1. In further embodiments, o is 2. In further embodiments, o is 3. In further embodiments, o is 4.

In some embodiments of the compound of formula (I), (Ia), (Ib), (Ic), or (Id), or a pharmaceutically acceptable salt or solvate thereof, the compound is substituted with an albumin binder. In further embodiments, P is substituted with an albumin binder. In further embodiments, L¹ is substituted with an albumin binder. In further embodiments, M is substituted with an albumin binder. In some embodiments, none of P, L¹, and M are substituted with an albumin binder (i.e., the compound of formula (I), (Ia), (Ib), (Ic), or (Id), or a pharmaceutically acceptable salt or solvate thereof is not substituted with an albumin binder).

In some embodiments, the HER2-targeting compound is a compound of formula (I):

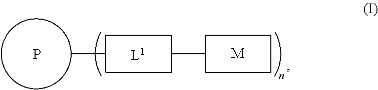

or a pharmaceutically acceptable salt or solvate thereof.

In some embodiments, the HER2-targeting compound is a compound of formula (Ib):

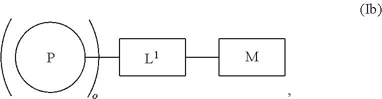

or a pharmaceutically acceptable salt or solvate thereof.
Cyclic Peptide (P) and Incorporation into, e.g., Compounds of Formulae (I), (Ia), (Ib), (Ic), and (Id)

In some aspects, the present disclosure provides a cyclic peptide (P) that is capable of binding to human epidermal growth factor receptor 2 (HER2), as well as compounds comprising said cyclic peptide (P).

In an embodiment, P binds to human epidermal growth factor receptor 2 (HER2) with a dissociation constant (K_{D}) of about 1000 nM or less as measured by surface plasmon resonance (SPR) at a temperature of 25° C.

In another embodiment, the K_{D} is about 200 nM or less.

In yet another embodiment, P comprises seven to fifteen amino acids and/or amino acid derivatives.

In still another embodiment, P comprises at least two consecutive amino acids or amino acid derivatives independently selected from tryptophan and tryptophan derivatives.

In one or more embodiments, P has the structure:

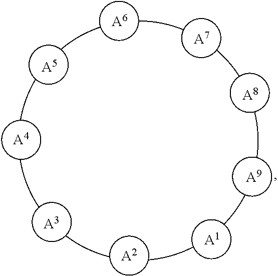

wherein:
A¹ is

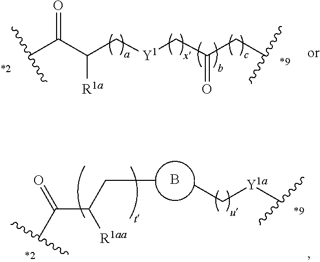

wherein:
R^{1a} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁₋₆-alkyl)₂, and —NH₂;
\textcircled{B} is selected from C₃₋₈-cycloalkyl, phenyl, and 5-6 membered heteroaryl, wherein the C₃₋₈-cycloalkyl, phenyl, and 5-6 membered heteroaryl of \textcircled{B} are optionally substituted with 1 or 2 substituents independently selected from halo, —OH, —NH₂, and C₁₋₆-alkyl;
R^{1aa} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁₋₆-alkyl)₂, and —NH₂;
Y¹ is selected from a bond, C≡C, NH, NC₁₋₆-alkyl, O, and S;
Y^{1a} is selected from C(O), NH, NC₁₋₆-alkyl, C(O)NH, C(O)NC₁₋₆-alkyl, NHC(O), N(C₁₋₆-alkyl) C(O), O, and S;
a is 1, 2, 3, or 4;
b, c, t′, and x′ are each independently 0 or 1;
u′ is 0, 1, 2, or 3; and
*2 indicates the point of attachment to A² and *9 indicates the point of attachment to A⁹;
A² is

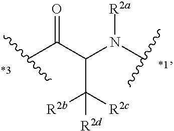

wherein:
R^{2a} is selected from H and C₁₋₆-alkyl;
R^{2b} is selected from H, C₁₋₆-alkyl, and halo;
R^{2c} is selected from halo, C₁₋₆-alkyl, C₃₋₈-cycloalkyl, phenyl, and 5-6 membered heteroaryl, wherein the C₁₋₆-alkyl, phenyl, and 5-6 membered heteroaryl of R^{2c} are optionally substituted with 1, 2, 3, or 4 substituents independently selected from halo, —C(O)NH₂, —C(O)OH, —C(O)C₁₋₆-alkyl, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —NHC₁₋₆-alkyl, —N(C₁₋₆-alkyl)₂, and —NH₂;
R^{2d} is selected from H, C₁₋₆-alkyl, and halo; and
*3 indicates the point of attachment to A³ and *1 indicates the point of attachment to A¹;
A³ is

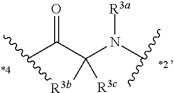

wherein:
R^{3a} is selected from H and C₁₋₆-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)-(5-6 membered heterocycloalkyl), and —C₁₋₆-alkyl-N(H)—C₁₋₆-alkyl-(5-6 membered heterocycloalkyl), wherein the C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)-(5-6 membered heterocycloalkyl), and —C₁₋₆-alkyl-N(H)—C₁₋₆-alkyl-(5-6 membered heterocycloalkyl) of R^{3c} are optionally substituted with 1, 2, 3, 4, 5, or 6 substituents independently selected from —CN, —C(O)OH, —C₁₋₆-alkyl-C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *4 indicates the point of attachment to A⁴ and *2 indicates the point of attachment to A²;
A⁴ is

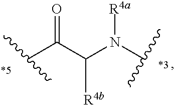

wherein:
R^{4a} is selected from H, C₁₋₆-alkyl, and —CH₂-phenyl, wherein the C₁₋₆-alkyl and —CH₂-phenyl of R^{4a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{4b} is selected from H, C₁₋₆-alkyl, —C₁₋₆-alkyl-phenyl, —C₁₋₆-alkyl-O-phenyl, —C₁₋₆-alkyl-(5-6 membered heteroaryl), —C₁₋₇-alkyl-C(O)OH, and —C₁₋₆-alkyl-NH—C(O)OH, wherein the C₁₋₆-alkyl, —C₁₋₆-alkyl-phenyl, —C₁₋₆-alkyl-O-phenyl, and —C₁₋₆-alkyl-(5-6 membered heteroaryl) of R^{4b} are optionally substituted with 1, 2, or 3 substituents independently selected from halo, —C(O)NH₂, —NH₂, —NHC₁₋₆-alkyl, —N(C₁₋₆-alkyl)₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —OC₁₋₆-alkyl, and C₁₋₆-alkyl; and
wherein *5 indicates the point of attachment to A⁵ and *3 indicates the point of attachment to A³;
A⁵ is

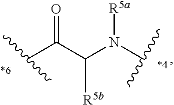

wherein:
R^{5a} is selected from H, C₁₋₆-alkyl, and —CH₂-phenyl, wherein the C₁₋₆-alkyl and —CH₂-phenyl of R^{5a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5b} is selected from H, C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c}, wherein the C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c} of R^{5b} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5c} is selected from H, C₁₋₆-alkyl, —NH₂, —NHC₁₋₆-alkyl, —N(C₁₋₆-alkyl)₂, —OH, —OC₁₋₆-alkyl, C₁₋₆-haloalkyl, C₁₋₆-alkyl-C(O)OH, and C₁₋₆-alkyl-C(O) OC₁₋₆-alkyl; and
wherein *6 indicates the point of attachment to A⁶ and *4 indicates the point of attachment to A⁴;
A⁶ is

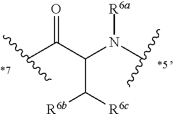

wherein:
R^{6a} is selected from H and C₁₋₆-alkyl;
R^{6b} is selected from H and C₁₋₆-alkyl;
R^{6c} is selected from C₁₋₆-alkyl and 5-10 membered heteroaryl, wherein the C₁₋₆-alkyl and 5-10 membered heteroaryl of R^{6c} are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —OC(O)—C₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *5 indicates the point of attachment to A⁵ and *7 indicates the point of attachment to A⁷;
A⁷ is

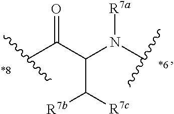

wherein:
R^{7a} is selected from H and C₁₋₆-alkyl;
R^{7b} is selected from H and C₁₋₆-alkyl;
R^{7c} is selected from C₁₋₆-alkyl and 5-10 membered heteroaryl, wherein the C₁₋₆-alkyl and 5-10 membered heteroaryl are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *8 indicates the point of attachment to A⁸ and *6 indicates the point of attachment to A⁶;
A⁸ is

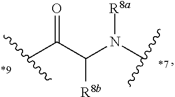

wherein:
R^{8a} is selected from H and C₁₋₆-alkyl;
R^{8b} is selected from H, C₁₋₆-alkyl, C₃₋₈-cycloalkyl, 5-7 membered heterocycloalkyl ring, and 5-10 membered heteroaryl, wherein the C₁₋₆-alkyl, C₃₋₈-cycloalkyl, 5-7 membered heterocycloalkyl ring, and 5-10 membered heteroaryl of R^{8b} are optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, and —OC₁₋₆-alkyl; and
wherein *9 indicates the point of attachment to A⁹ and *7 indicates the point of attachment to A⁷;
A⁹ is

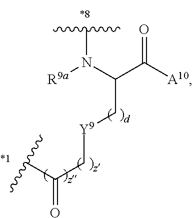

wherein:
Y⁹ is selected from a bond, C(O), NH, NC₁₋₆-alkyl, O, and S;
R^{9a} is selected from H and C₁₋₆-alkyl;
d is 1, 2, or 3; and
z′ and z″ are each independently 0 or 1; and
wherein *8 indicates the point of attachment to A⁸ and *1 indicates the point of attachment to A¹; and
A¹⁰ is

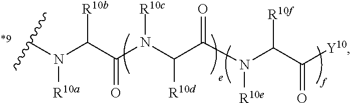

wherein:
Y¹⁰ is selected from OH and N(R^{10g})(R^{10h});
R^{10a}, R^{10c}, R^{10e}, R^{10g}, and R^{10h} are each independently selected from H and C₁₋₆-alkyl;
R^{10b} is selected from H, C₁₋₆-alkyl, —C₁₋₆-alkyl-phenyl, —C₁₋₆-alkyl-(5-6 membered heteroaryl), —C₁₋₇-alkyl-C(O)R¹⁰ⁱ, —C₁₋₆-alkyl-O—C(O)R¹⁰ⁱ, and —C₁₋₆-alkyl-NH—C(O)R¹⁰ⁱ, wherein the C₁₋₆-alkyl, —C₁₋₆-alkyl-phenyl, and —C₁₋₆-alkyl-(5-6 membered heteroaryl) of R^{10b} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10a} and R^{10b}, together with the atoms to which they are attached, form a 5-7 membered heterocycloalkyl ring;
R^{10d} is selected from H, C₁₋₆-alkyl, —C₁₋₆-alkyl-phenyl, —C₁₋₆-alkyl-(5-6 membered heteroaryl), —C₁₋₇-alkyl-C(O)R^{10j}, —C₁₋₆-alkyl-O—C(O)R^{10j}, and —C₁₋₆-alkyl-NH—C(O)R^{10j}, wherein the C₁₋₆-alkyl, —C₁₋₆-alkyl-phenyl, and —C₁₋₆-alkyl-(5-6 membered heteroaryl) of R^{10d} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, —C₁₋₆-alkyl-(5-6 membered heteroaryl), —C₁₋₇-alkyl-C(O)R^{10k}, —C₁₋₆-alkyl-O—C(O)R^{10k}, and —C₁₋₆-alkyl-NH—C(O)R^{10k} wherein the C₁₋₆-alkyl, —C₁₋₆-alkyl-phenyl, and —C₁₋₆-alkyl-(5-6 membered heteroaryl) of R^{10f} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10e} and R^{10f}, together with the atoms to which they are attached, form a 5-7 membered heterocycloalkyl ring;
R¹⁰ⁱ, R^{10j}, and R^{10k} are each independently selected from H, C₁₋₆-alkyl, C₃₋₇ cycloalkyl, 5-6 membered heteroaryl, and 3-7 membered heterocycloalkyl, wherein the C₃₋₇ cycloalkyl, 5-6 membered heteroaryl, and 3-7 membered heterocycloalkyl of R¹⁰ⁱ, R^{10j}, and R^{10k} are each optionally substituted with 1, 2, 3, or 4 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, C₁₋₆-alkyl, —C(O)OH, —C(O)NH₂, and C₁₋₆-alkyl-C(O)NH₂;
e and f are each independently 0 or 1; and
wherein *9 indicates the point of attachment to A⁹.

In some embodiments of the cyclic peptide (P), or compounds comprising cyclic peptide (P):
A¹ is

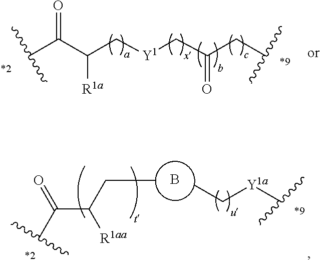

wherein:
R^{1a} is selected from H, C₁₋₆-alkyl, OH, halo, —NH₂, —N(H)—C(O)—C₁₋₆-alkyl, —NHC₁₋₆-alkyl, and —N(C₁₋₆-alkyl)₂, wherein the C₁₋₆-alkyl, —N(H)—C(O)—C₁₋₆-alkyl, —NHC₁₋₆-alkyl, and —N(C₁₋₆-alkyl) 2 of R^{1a} are optionally substituted with 1, 2, or 3 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁₋₆-alkyl)₂, and —NH₂;
\textcircled{B} is selected from C₃₋₈-cycloalkyl and phenyl, wherein the C₃₋₈-cycloalkyl and phenyl of \textcircled{B} are optionally substituted with 1 or 2 substituents independently selected from C₁₋₆-alkyl, halo, —OH, and —NH₂;
R^{1aa} is selected from H, C₁₋₆-alkyl, —NH₂, —NHC₁₋₆-alkyl, and —N(C₁₋₆-alkyl)₂;
Y¹ is selected from a bond, C≡C, NH, NC₁₋₆-alkyl, O, and S;
Y^{1a} is selected from C(O), NH, NC₁₋₆-alkyl, C(O)NH, NHC(O), O, and S;
a is 1, 2, 3, or 4;
b, c, t′, and x′ are each independently 0 or 1;
u′ is 0, 1, 2, or 3;
*2 indicates the point of attachment to A² and *9 indicates the point of attachment to A⁹;
A² is

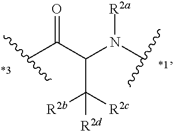

wherein:
R^{2a} is selected from H and C₁₋₃-alkyl; R^{2b} is selected from H, C₁₋₆-alkyl, and halo;
R^{2c} is selected from C₁₋₆-alkyl, phenyl, and 5-6 membered heteroaryl, wherein the phenyl and 5-6 membered heteroaryl of R^{2c} are optionally substituted with 1, 2, or 3, substituents independently selected from halo, —C(O)NH₂, —C(O)OH, —C(O)C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —NHC₁₋₆-alkyl, —N(C₁₋₆-alkyl)₂, and —NH₂;
R^{2d} is selected from H, C₁₋₆-alkyl, and halo;
*3 indicates the point of attachment to A³ and *1 indicates the point of attachment to A¹;
A³ is

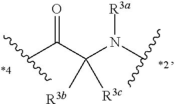

wherein:
R^{3a} is selected from H and C₁₋₃-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)-(5-6 membered heterocycloalkyl), and —C₁₋₆-alkyl-N(H)—C₁₋₆-alkyl-(5-6 membered heterocycloalkyl), wherein the C₁₋₆-alkyl, —C₁₋₆-alkyl-C(O)NH—C₁₋₆-alkyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)-(5-6 membered heterocycloalkyl), and —C₁₋₆-alkyl-N(H)—C₁₋₆-alkyl-(5-6 membered heterocycloalkyl) of R^{3c} are optionally substituted with 1, 2, 3, 4, or 5 substituents independently selected from —C(O)OH, —C₁₋₆-alkyl-C(O)OH, —C(O)NH₂, —NH₂, —NHC₁₋₆-alkyl, —N(C₁₋₆-alkyl)₂, —NHC(O)C₁₋₆-alkyl, —OH, and —OC₁₋₆-alkyl;
wherein *4 indicates the point of attachment to A⁴ and *2 indicates the point of attachment to A²;
A⁴ is

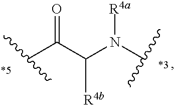

wherein:
R^{4a} is selected from H and C₁₋₃-alkyl, wherein the C₁₋₃-alkyl of R^{4a} is optionally substituted with 1 or 2 substituents independently selected from —NH₂, —NHC₁₋₆-alkyl, —N(C₁-6-alkyl)₂, —OH, and —OC₁₋₆-alkyl;
R^{4b} is selected from H, C₁₋₆-alkyl, —C₁₋₆-alkyl-phenyl, —C₁₋₆-alkyl-O-phenyl, —C₁₋₆-alkyl-pyridyl, —C₁₋₇-alkyl-C(O)OH, and —C₁₋₆-alkyl-NH—C(O)OH, wherein the C₁₋₆-alkyl, —C₁-6-alkyl-phenyl, —C₁₋₆-alkyl-O-phenyl, and —C₁₋₆-alkyl-pyridyl, of R^{4b} are optionally substituted with 1 or 2 substituents independently selected from halo, —C(O)NH₂, —NH₂, —NHC₁₋₆-alkyl, —N(C₁₋₆-alkyl)₂, —OH, and —OC₁₋₆-alkyl;
wherein *5 indicates the point of attachment to A⁵ and *3 indicates the point of attachment to A³;
A⁵ is

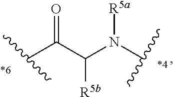

wherein:
R^{5a} is selected from H and C₁₋₃-alkyl, wherein the C₁₋₃-alkyl of R^{5a} is optionally substituted with 1 or 2 substituents independently selected from —NH₂, —NHC₁₋₆-alkyl, —N(C₁-6-alkyl)₂, and —OH;
R^{5b} is selected from H, C₁₋₆-alkyl, —C₁₋₇-alkyl-C(O)R^{5c}, —C₁₋₆-alkyl-O—C(O)R^{5c}, and —C₁₋₆-alkyl-NH—C(O)R^{5c}, wherein the C₁₋₆-alkyl, —C₁₋₇-alkyl-C(O)R^{5c}, —C₁₋₆-alkyl-O—C(O)R^{5c}, and —C₁₋₆-alkyl-NH—C(O)R^{5c} of R^{5b} are each optionally substituted with 1 or 2 substituents independently selected from —NH₂, —NHC₁₋₆-alkyl, —N(C₁₋₆-alkyl)₂, and —OH;
R^{5c} is selected from H, C₁₋₆-alkyl, —NH₂, —OH, —OC₁₋₆-alkyl, C₁₋₆-haloalkyl, C₁₋₆-alkyl-C(O)OH, and C₁₋₆-alkyl-C(O) OC₁₋₆-alkyl;
wherein *6 indicates the point of attachment to A⁶ and *4 indicates the point of attachment to A⁴;
A⁶ is

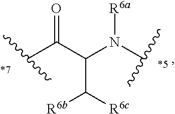

wherein:
R^{6a} is selected from H and C₁₋₃-alkyl;
R^{6b} is selected from H and C₁₋₆-alkyl;
R^{6c} is selected from C₁₋₆-alkyl and 7-10 membered heteroaryl, wherein the C₁₋₆-alkyl and 7-10 membered heteroaryl of R^{60c} are each optionally substituted with 1, 2, or 3 substituents independently selected from C₁₋₆-alkyl, —CN, halo, —NH₂, —NHC₁₋₆-alkyl, —N(C₁₋₆-alkyl)₂, —OH, —OC₁₋₆-alkyl, and —OC(O)—C₁₋₆-alkyl;
wherein *5 indicates the point of attachment to A⁵ and *7 indicates the point of attachment to A⁷;
A⁷ is

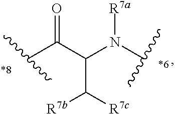

wherein:
R^{7a} is selected from H and C₁₋₃-alkyl;
R^{7b} is selected from H and C₁₋₆-alkyl;
R^{7c} is selected from C₁₋₆-alkyl and 7-10 membered heteroaryl, wherein the C₁₋₆-alkyl and 7-10 membered heteroaryl are each optionally substituted with 1, 2, or 3 substituents independently selected from —CN, halo, —NH₂, —NHC₁₋₆-alkyl, —N(C₁₋₆-alkyl)₂, and —OH;
wherein *8 indicates the point of attachment to A⁸ and *6 indicates the point of attachment to A⁶;
A⁸ is

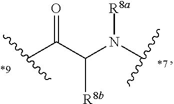

wherein:
R^{8a} is selected from H and C₁₋₃-alkyl;
R^{8b} is selected from H, C₁₋₆-alkyl, C₃₋₆-cycloalkyl, 5-6 membered heteroaryl, wherein the C₁₋₆-alkyl, C₃₋₆-cycloalkyl and 5-6 membered heteroaryl of R^{8b} are optionally substituted with 1, 2, or 3 substituents independently selected from —CN, halo, —NH₂, —NHC₁₋₆-alkyl, —N(C₁₋₆-alkyl)₂, and —OH;
wherein *9 indicates the point of attachment to A⁹ and *7 indicates the point of attachment to A⁷;
A⁹ is

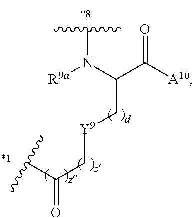

wherein:
Y⁹ is selected from a bond, C(O), NH, NC₁₋₆-alkyl, O, and S;
R^{9a} is selected from H and C₁₋₃-alkyl;
d is 1, 2, or 3;
z′ and z″ are each independently 0 or 1;
A¹⁰ is

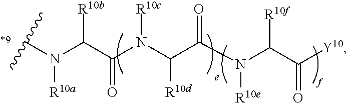

wherein:
Y¹⁰ is selected from OH and N(R^{10g})(R^{10h});
R^{10a}, R^{10c}, and R^{10e} are each independently selected from H and C₁₋₃-alkyl;
R^{10g} and R^{10h} are each independently selected from H and C₁₋₆-alkyl;
R^{10b} is selected from H, C₁₋₆-alkyl, —C₁₋₆-alkyl-phenyl, and —C₁₋₆-alkyl-NH—C(O)R¹⁰ⁱ, wherein the C₁₋₆-alkyl and —C₁₋₆-alkyl-phenyl of R^{10b} are optionally substituted with 1 or 2 substituents independently selected from halo, —NH₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10a} and R^{10b}, together with the atoms to which they are attached, form a 5-7 membered heterocycloalkyl ring;
R^{10d} is selected from H, C₁₋₆-alkyl, —C₁₋₆-alkyl-phenyl, and —C₁₋₆-alkyl-NH—C(O)R^{10j}, wherein the C₁₋₆-alkyl and —C₁₋₆-alkyl-phenyl of R^{10d} are optionally substituted with 1 or 2 substituents independently selected from halo, —NH₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, and —C₁₋₆-alkyl-NH—C(O)R^{10k}, wherein the C₁₋₆-alkyl, —C₁₋₆-alkyl-phenyl, and —C₁₋₆-alkyl-(5-6 membered heteroaryl) of R^{10f} are optionally substituted with 1 or 2 substituents independently selected from halo, —NH₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10e} and R^{10f}, together with the atoms to which they are attached, form a 5-7 membered heterocycloalkyl ring;
R¹⁰ⁱ, R^{10j}, and R^{10k} are each independently selected from H, C₁₋₆-alkyl, 5-6 membered heterocycloalkyl, and 5-6 membered heteroaryl, wherein the 5-6 membered heterocycloalkyl, and 5-6 membered heteroaryl of R¹⁰ⁱ, R^{10j}, and R^{10k} are each optionally substituted with 1, 2, or 3 substituents independently selected from halo, —NH₂, —NHC₁₋₆-alkyl, —N(C₁₋₆-alkyl)₂, —N₃, —OH, C₁₋₆-alkyl, and C₁₋₆-alkyl-C(O)OH;
e and f are each independently 0 or 1; and
wherein *9 indicates the point of attachment to A⁹.

In some embodiments of the cyclic peptide (P), or compounds comprising cyclic peptide (P):
A¹ is

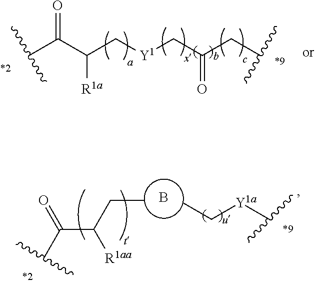

wherein:
R^{1a} is selected from H, C₁₋₆-alkyl, halo, —NH₂, —N(H)—C(O)—C₁₋₆-alkyl, —NHC₁₋₆-alkyl, and —N(C₁₋₆-alkyl)₂, wherein the C₁₋₆-alkyl, —N(H)—C(O)—C₁₋₆-alkyl, —NHC₁₋₆-alkyl, and —N(C₁₋₆-alkyl) 2 of R^{1a} are optionally substituted with 1 or 2 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, 5-membered heteroaryl, —OH, and —NH₂;
\textcircled{B} is selected from C₅₋₇-cycloalkyl and phenyl, wherein the C₅₋₇-cycloalkyl and phenyl of \textcircled{B} are optionally substituted with 1 substituent selected from C₁₋₆-alkyl, halo, —OH, and —NH₂;
R^{1aa} is selected from H, C₁₋₆-alkyl, —NHC₁₋₆-alkyl, —N(C₁₋₆-alkyl)₂, and —NH₂;
Y¹ is selected from a bond, C≡C, NH, NC₁₋₆-alkyl, O, and S;
Y^{1a} is selected from C(O), NH, NC₁₋₆-alkyl, C(O)NH, NHC(O), O, and S;
a is 1, 2, or 3;
b, c, t′, and x′ are each independently 0 or 1;
u′ is 0, 1, or 2;
*2 indicates the point of attachment to A² and *9 indicates the point of attachment to A⁹
A² is

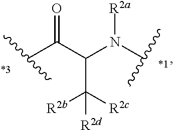

wherein:
R^{2a} is selected from H and C₁₋₃-alkyl;
R^{2b} is selected from H, C₁₋₆-alkyl, and halo;
R^{2c} is selected from C₁₋₆-alkyl, phenyl, and 6-membered heteroaryl, wherein the phenyl and 6-membered heteroaryl of R^{2c} are optionally substituted with 1 or 2 substituents independently selected from halo, —C(O)NH₂, —C(O)OH, —C(O)C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, and —NH₂;
R^{2d} is selected from H, C₁₋₆-alkyl, and halo; and
*3 indicates the point of attachment to A³ and *1 indicates the point of attachment to A¹;
A³ is

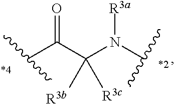

wherein:
R^{3a} is selected from H and C₁₋₃-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-(6-membered heterocycloalkyl), —C₁₋₆-alkyl-N(H)-(6-membered heterocycloalkyl), —C₁₋₆-alkyl-N(H)—C(O)-(6-membered heterocycloalkyl), and —C₁₋₆-alkyl-N(H)—C₁₋₆-alkyl-(6-membered heterocycloalkyl), wherein the C₁₋₆-alkyl, —C₁₋₆-alkyl-C(O)NH—C₁₋₆-alkyl, —C₁₋₆-alkyl-(6-membered heterocycloalkyl), —C₁₋₆-alkyl-N(H)-(6-membered heterocycloalkyl), —C₁₋₆-alkyl-N(H)—C(O)-(6-membered heterocycloalkyl), and —C₁₋₆-alkyl-N(H)—C₁₋₆-alkyl-(6-membered heterocycloalkyl) of R^{3c} are optionally substituted with 1 2, 3, 4, or 5 substituents independently selected from —C(O)OH, —C₁₋₆-alkyl-C(O)OH, —C(O)NH₂, —NH₂, —NHC(O)C₁₋₆-alkyl, —OH, and —OC₁₋₆-alkyl; and
wherein *4 indicates the point of attachment to A⁴ and *2 indicates the point of attachment to A²;
A⁴ is

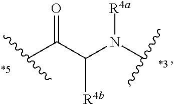

wherein:
R^{4a} is selected from H and C₁₋₃-alkyl, wherein the C₁₋₃-alkyl of R^{4a} is optionally substituted with 1 substituent selected from —NH₂, —OH, —OC₁₋₆-alkyl, and C₁₋₆-alkyl;
R^{4b} is selected from H, C₁₋₆-alkyl, —C₁₋₆-alkyl-phenyl, —C₁₋₆-alkyl-pyridyl, and —C₁₋₆-alkyl-NH—C(O)OH, wherein the C₁₋₆-alkyl, —C₁₋₆-alkyl-phenyl, and —C₁₋₆-alkyl-pyridyl of R^{4b} are optionally substituted with 1 or 2 substituents independently selected from halo, —NH₂, —OH, and —OC₁₋₆-alkyl; and
wherein *5 indicates the point of attachment to A⁵ and *3 indicates the point of attachment to A³;
A⁵ is

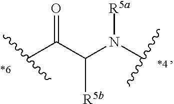

wherein:
R^{5a} is selected from H and C₁₋₃-alkyl, wherein the C₁₋₃-alkyl of R^{5a} is optionally substituted with 1 substituent selected from —NH₂, —NHC₁₋₆-alkyl, —N(C₁₋₆-alkyl)₂, and —OH;
R^{5b} is selected from H, C₁₋₆-alkyl, —C₁₋₆-alkyl-O—C(O)R^{5c}, and —C₁₋₆-alkyl-NH—C(O)R^{5c}, wherein the C₁₋₆-alkyl, —C₁₋₆-alkyl-O—C(O)R^{5c}, and —C₁₋₆-alkyl-NH—C(O)R^{5c} of R^{5b} are each optionally substituted with 1 or 2 substituents independently selected from —NH₂, —NHC₁₋₆-alkyl, —N(C₁₋₆-alkyl)₂, and —OH;
R^{5c} is selected from H, C₁₋₆-alkyl, —NH₂, —OH, —OC₁₋₆-alkyl, C₁₋₆-haloalkyl, C₁₋₆-alkyl-C(O)OH, and C₁₋₆-alkyl-C(O) OC₁₋₆-alkyl; and
wherein *6 indicates the point of attachment to A⁶ and *4 indicates the point of attachment to A⁴;
A⁶ is

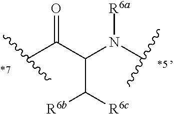

wherein:
R^{6a} is selected from H and C₁₋₃-alkyl;
R^{6b} is selected from H and C₁₋₆-alkyl;
R^{6c} is selected from C₁₋₆-alkyl and 8-9 membered heteroaryl, wherein the C₁₋₆-alkyl and 8-9 membered heteroaryl of R^{6c} are each optionally substituted with 1 or 2 substituents independently selected from C₁₋₆-alkyl, —CN, halo, —NH₂, —OH, —OC₁₋₆-alkyl, and —OC(O)—C₁₋₆-alkyl; and
wherein *5 indicates the point of attachment to A⁵ and *7 indicates the point of attachment to A⁷;
A⁷ is

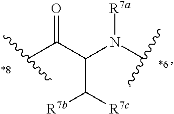

wherein:
R^{7a} is selected from H and C₁₋₃-alkyl;
R^{7b} is selected from H and C₁₋₆-alkyl;
R^{7c} is selected from C₁₋₆-alkyl and 8-9 membered heteroaryl, wherein the C₁₋₆-alkyl and 8-9 membered heteroaryl are each optionally substituted with 1 or 2 substituents independently selected from —CN, halo, —NH₂, and —OH; and
wherein *8 indicates the point of attachment to A⁸ and *6 indicates the point of attachment to A⁶;
A⁸ is

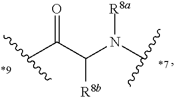

wherein:
R^{8a} is selected from H and C₁₋₃-alkyl;
R^{8b} is selected from H, C₁₋₆-alkyl, and C₃₋₆-cycloalkyl, wherein the C₁₋₆-alkyl and C₃-6-cycloalkyl of R^{8b} are optionally substituted with 1 or 2 substituents independently selected from —CN, halo, —NH₂, and —OH; and
wherein *9 indicates the point of attachment to A⁹ and *7 indicates the point of attachment to A⁷;
A⁹ is

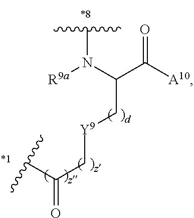

wherein:
Y⁹ is selected from a bond, C(O), NH, NC₁₋₆-alkyl, O, and S;
R^{9a} is selected from H and C₁₋₃-alkyl;
d is 1 or 2; and
wherein *8 indicates the point of attachment to A⁸ and *1 indicates the point of attachment to A¹; and
A¹⁰ is

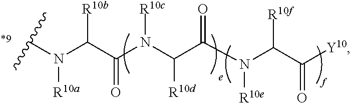

wherein:
Y¹⁰ is selected from OH and N(R^{10g})(R^{10h});
R^{10a}, R^{10c}, and R^{10e} are each independently selected from H and C₁₋₃-alkyl;
R^{10g} and R^{10h} are each independently selected from H and C₁₋₆-alkyl;
R^{10b} is selected from H, C₁₋₃-alkyl, —C₁₋₆-alkyl-phenyl, and —C₁₋₆-alkyl-NH—C(O)R¹⁰ⁱ, wherein the C₁₋₆-alkyl and —C₁₋₆-alkyl-phenyl of R^{10b} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
R^{10d} is selected from H, C₁₋₆-alkyl, —C₁₋₆-alkyl-phenyl, and —C₁₋₆-alkyl-NH—C(O)R^{10j}, wherein the C₁₋₆-alkyl and —C₁₋₆-alkyl-phenyl of R^{10d} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, and —C₁₋₆-alkyl-NH—C(O)R^{10k}, wherein the C₁₋₆-alkyl, —C₁₋₆-alkyl-phenyl, and —C₁₋₆-alkyl-(5-6 membered heteroaryl) of R^{10f} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
R¹⁰ⁱ, R^{10j}, and R^{10k} are each independently selected from H, C₁₋₆-alkyl, 6-membered heterocycloalkyl, and 6-membered heteroaryl, wherein the 6-membered heterocycloalkyl, and 6-membered heteroaryl of R¹⁰ⁱ, R^{10j}, and R^{10k} are each optionally substituted with 1 or 2 substituents independently selected from halo, —NH₂, —NHC₁₋₆-alkyl, —N(C₁₋₆-alkyl)₂, —OH, C₁-6-alkyl, and C₁₋₆-alkyl-C(O)OH;
e and f are each independently 0 or 1; and
wherein *9 indicates the point of attachment to A⁹.

In some embodiments of the cyclic peptide (P), or compounds comprising cyclic peptide (P):

A¹ is a moiety of formula (A1-I), formula (A1-II), formula (A1-III), or formula (A1-IV)

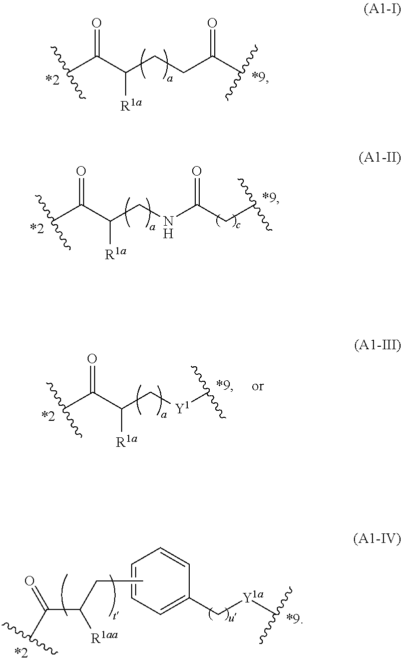

In some embodiments of the cyclic peptide (P), or compounds comprising cyclic peptide (P):

A¹ is substituted with one PEG chain.

In some embodiments of the cyclic peptide (P), or compounds comprising cyclic peptide (P):

A¹ is substituted with a PEG chain, wherein A¹ and the PEG chain, together, have the structure of formula (A1-P):

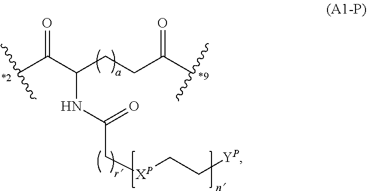

wherein:
X^{P} is selected from CH₂, N(R^{P}), and O;
Y^{P} is selected from H, —OH, —OC₁₋₆-alkyl, and —N(R^{P})₂;
each R^{P} is independently selected from H and C₁₋₆-alkyl;
a is 1, 2, or 3;
n′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
r′ is 0, 1, 2, 3, or 4.

In some embodiments of the cyclic peptide (P), or compounds comprising cyclic peptide (P):

A¹ is selected from:

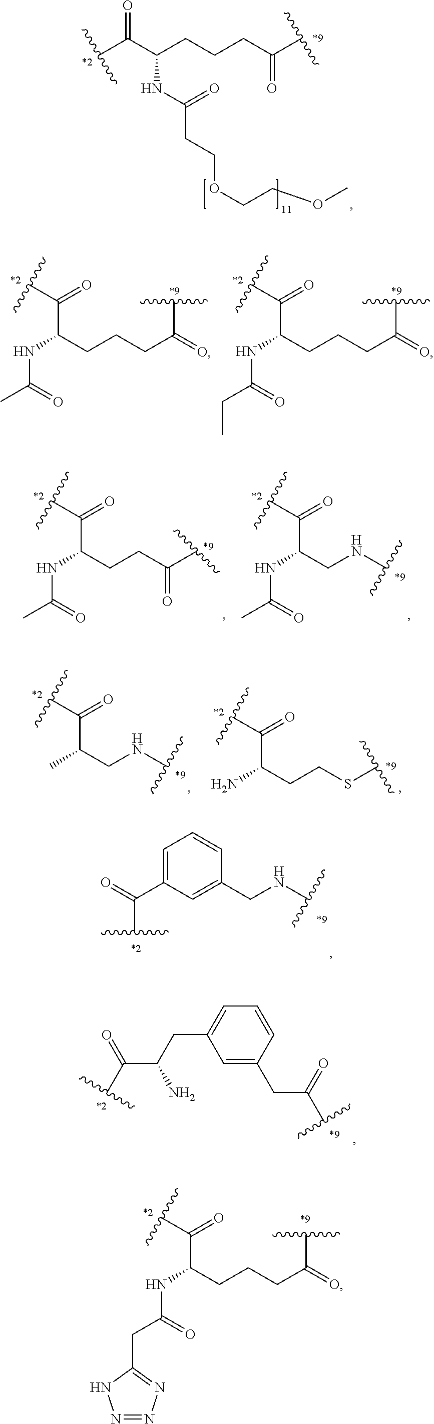

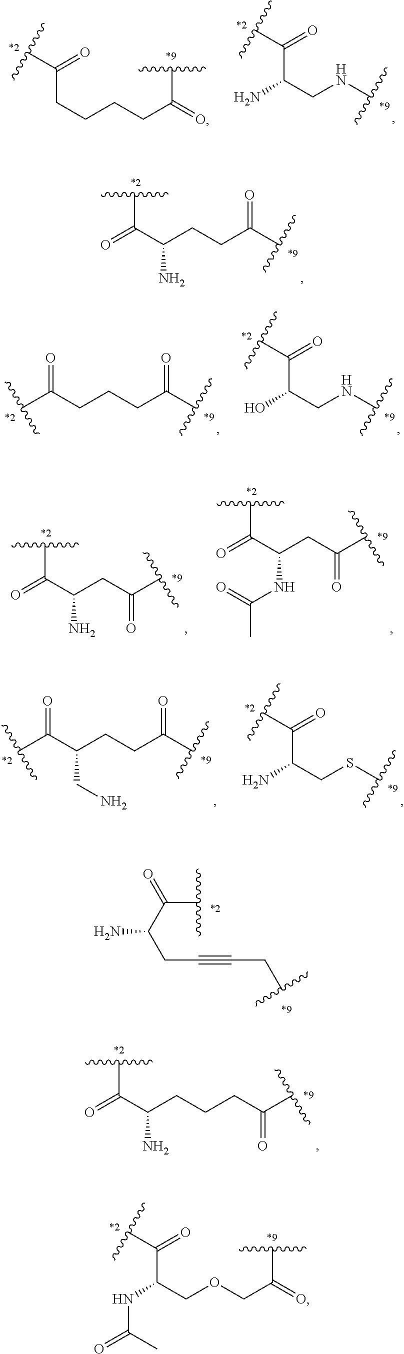

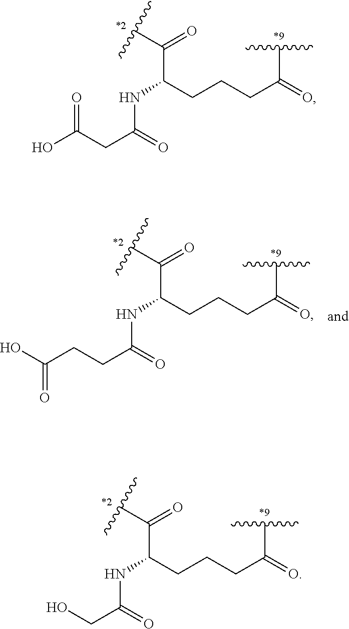 [SMILES: *C(=O)CCC[C@H](NC(=O)CC(=O)O)C(*)=O ; *C(=O)CCC[C@H](NC(=O)CCC(=O)O)C(*)=O ; *C(=O)CCC[C@H](NC(=O)CO)C(*)=O]

In some embodiments of the cyclic peptide (P), or compounds comprising cyclic peptide (P):

A¹ is selected from:

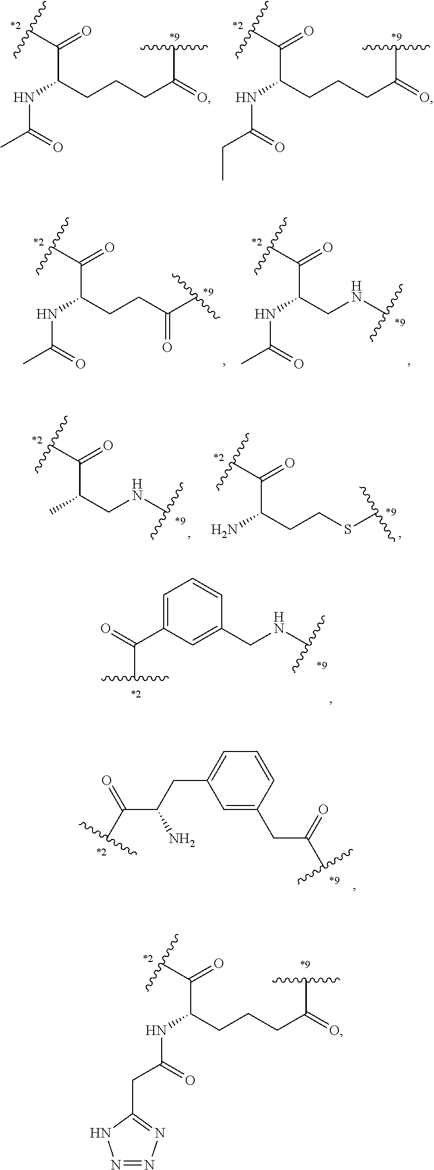

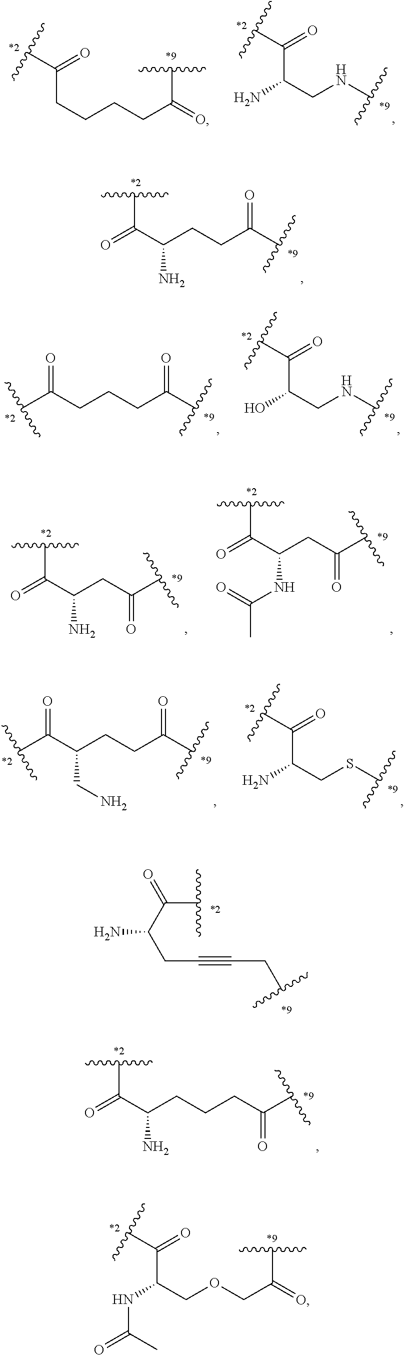

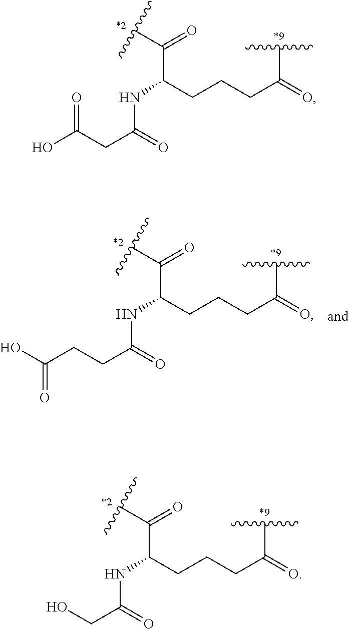 [SMILES: *C(=O)CCC[C@H](NC(=O)CC(=O)O)C(*)=O ; *C(=O)CCC[C@H](NC(=O)CCC(=O)O)C(*)=O ; *C(=O)CCC[C@H](NC(=O)CO)C(*)=O]

In some embodiments of the cyclic peptide (P), or compounds comprising cyclic peptide (P):

A¹ is selected from:

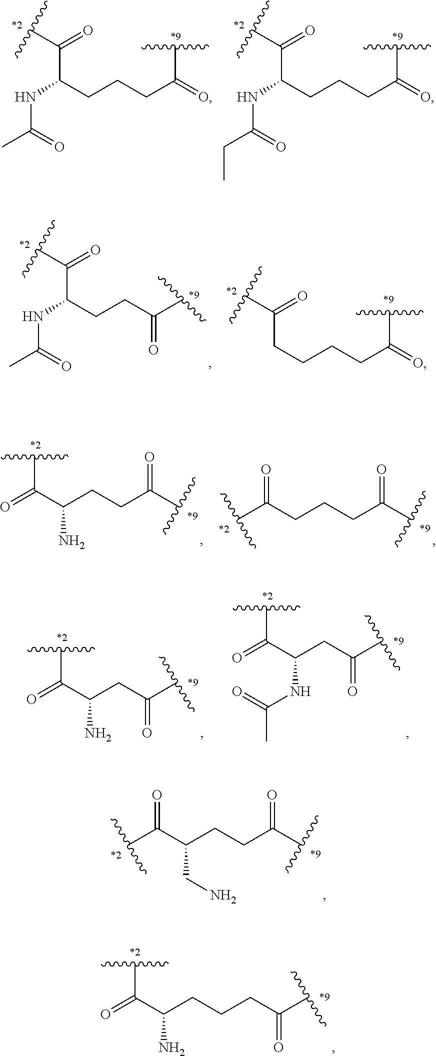

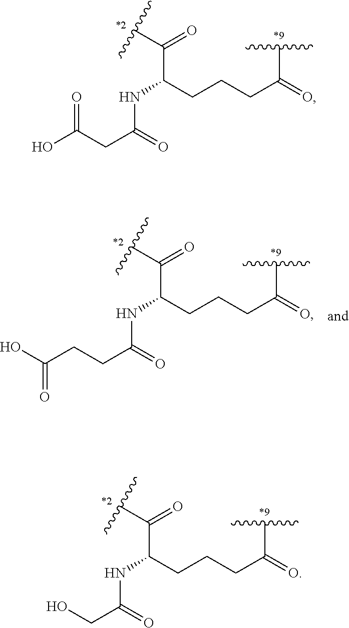 [SMILES: *C(=O)CCC[C@H](NC(=O)CC(=O)O)C(*)=O ; *C(=O)CCC[C@H](NC(=O)CCC(=O)O)C(*)=O ; *C(=O)CCC[C@H](NC(=O)CO)C(*)=O]

In some embodiments of the cyclic peptide (P), or compounds comprising cyclic peptide (P):

A¹ is selected from:

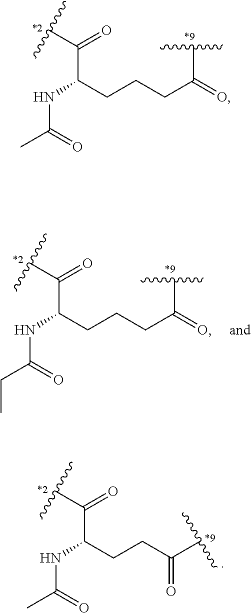 [SMILES: *C(=O)CCC[C@H](NC(C)=O)C(*)=O ; *C(=O)CCC[C@H](NC(=O)CC)C(*)=O ; "and" ; *C(=O)CC[C@H](NC(C)=O)C(*)=O]

In some embodiments of the cyclic peptide (P), or compounds comprising cyclic peptide (P):
A² is a moiety of formula (A2-I):

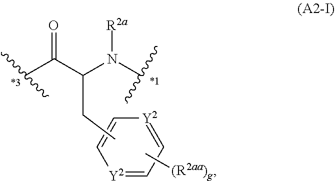

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, and —NH₂; and
g is 0, 1, or 2.

In some embodiments of the cyclic peptide (P), or compounds comprising cyclic peptide (P):

A² is in the L configuration.

In some embodiments of the cyclic peptide (P), or compounds comprising cyclic peptide (P):
A² is a moiety of formula (A2-Ia):

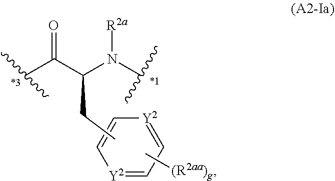

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2.

In some embodiments of the cyclic peptide (P), or compounds comprising cyclic peptide (P):

A² is selected from:

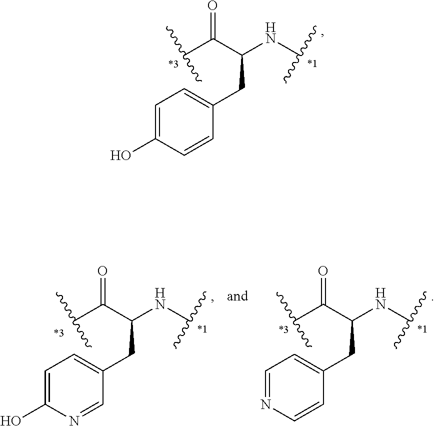 [SMILES: *N[C@@H](Cc1ccc(O)cc1)C(*)=O ; *N[C@@H](Cc1ccc(O)nc1)C(*)=O ; *N[C@@H](Cc1ccncc1)C(*)=O]

In some embodiments of the cyclic peptide (P), or compounds comprising cyclic peptide (P):
A³ is a moiety of formula (A3-I) or formula (A3-II):

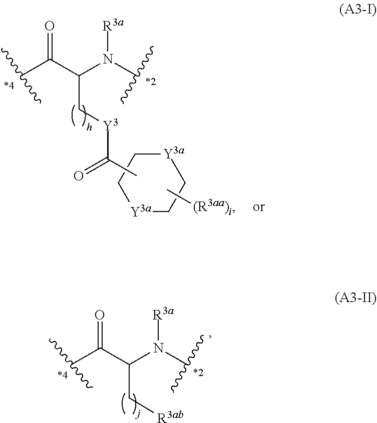

wherein:
Y³ is selected from a bond and NH;
each Y^{3a} is selected from NH and CH₂, provided that at least one Y^{3a} is NH;
R^{3a} is selected from H and C₁₋₃-alkyl;
each R^{3aa} is independently selected from —C(O)OH, —C₁₋₆-alkyl-C(O)OH, —C(O)NH₂, —NH₂, —NHC(O)C₁₋₆-alkyl, and —OC₁₋₆-alkyl;
R^{3ab} is selected from H, —OH, —C(O)OH, —C₁₋₆-alkyl-C(O)OH, —C(O)NH₂, —NH₂, —NHC(O)C₁₋₆-alkyl, —C(O)NHC₁₋₆-alkyl, and —OC₁₋₆-alkyl, wherein the —C(O)NHC₁₋₆-alkyl of R^{3ab} is optionally substituted with 1, 2, 3, 4, or 5 substituents independently selected from halo and —OH;
h is 1, 2, 3, 4, 5, or 6;
i is 0, 1, or 2; and
j is 1, 2, 3, 4, 5, or 6.

In some embodiments of the cyclic peptide (P), or compounds comprising cyclic peptide (P):

A³ is in the L configuration.

In some embodiments of the cyclic peptide (P), or compounds comprising cyclic peptide (P):
A³ is a moiety of formula (A3-I) or formula (A3-IIa):

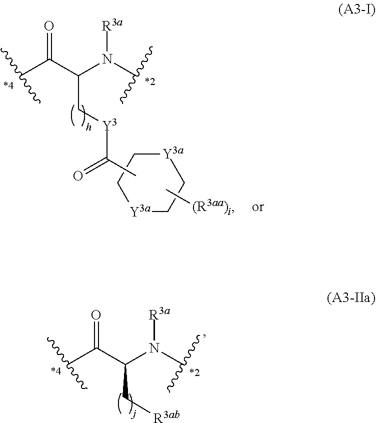

wherein:
Y³ is selected from a bond and NH;
each Y^{3a} is selected from NH and CH₂, provided that at least one Y^{3a} is NH;
R^{3a} is selected from H and C₁₋₃-alkyl;
each R^{3aa} is independently selected from —C(O)OH, —C₁₋₆-alkyl-C(O)OH, —C(O)NH₂, —NH₂, —NHC(O)C₁₋₆-alkyl, and —OC₁₋₆-alkyl;
R^{3ab} is selected from H, —OH, —C(O)OH, —C₁₋₆-alkyl-C(O)OH, —C(O)NH₂, —NH₂, —NHC(O)C₁₋₆-alkyl, —C(O)NHC₁₋₆-alkyl, and —OC₁₋₆-alkyl, wherein the —C(O)NHC₁₋₆-alkyl of R^{3ab} is optionally substituted with 1, 2, 3, 4, or 5 substituents independently selected from halo and —OH;
h is 1, 2, 3, 4, 5, or 6;
i is 0, 1, or 2; and
j is 1, 2, 3, 4, 5, or 6.

In some embodiments of the cyclic peptide (P), or compounds comprising cyclic peptide (P):
A³ is a moiety of formula (A3-IIa):

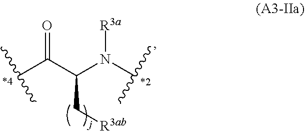

wherein:
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl; and
j is 1, 2, 3, 4, 5, or 6.

In some embodiments of the cyclic peptide (P), or compounds comprising cyclic peptide (P):

A³ is selected from:

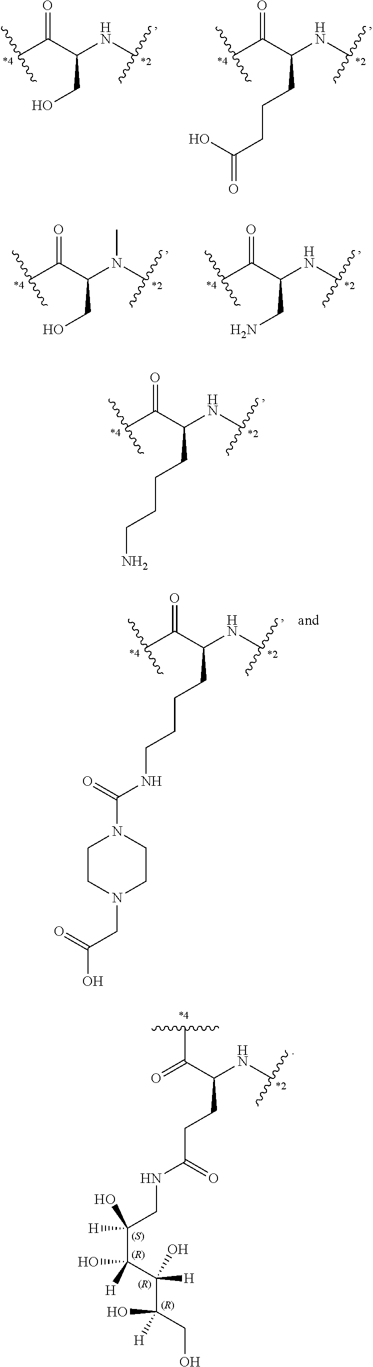

In some embodiments of the cyclic peptide (P), or compounds comprising cyclic peptide (P):

A³ is selected from:

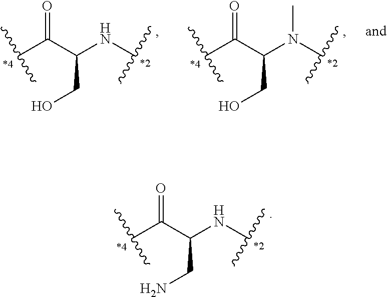

In some embodiments of the cyclic peptide (P), or compounds comprising cyclic peptide (P):
A⁴ is a moiety of formula (A4-I) or formula (A4-II):

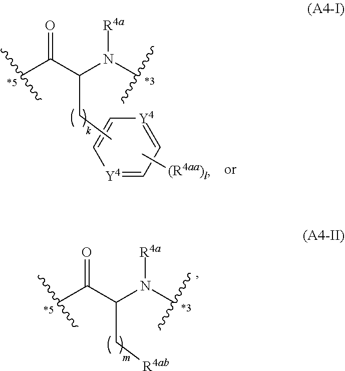

wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
R^{4ab} is selected from H, halo, —NH₂, —OH, —OC₁₋₆-alkyl, and —O-phenyl;
k is 1, 2, 3, 4, 5, or 6;
l is 0, 1, or 2; and
m is 1, 2, 3, 4, 5, or 6.

In some embodiments of the cyclic peptide (P), or compounds comprising cyclic peptide (P):

A⁴ is selected from:

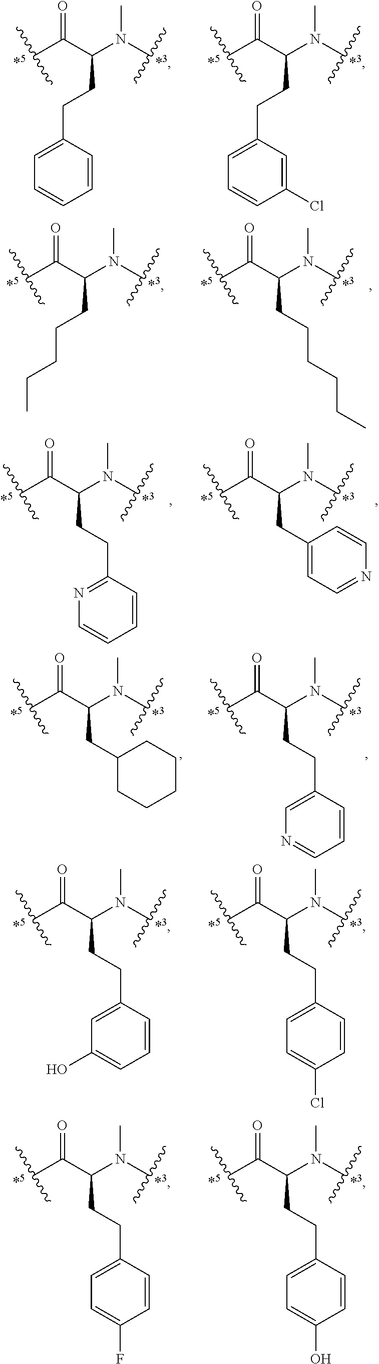

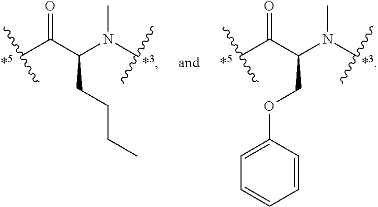 [SMILES: *C(=O)[C@H](CCCC)N(*)C ; *C(=O)[C@H](COc1ccccc1)N(*)C]

In some embodiments of the cyclic peptide (P), or compounds comprising cyclic peptide (P):

A⁴ is selected from:

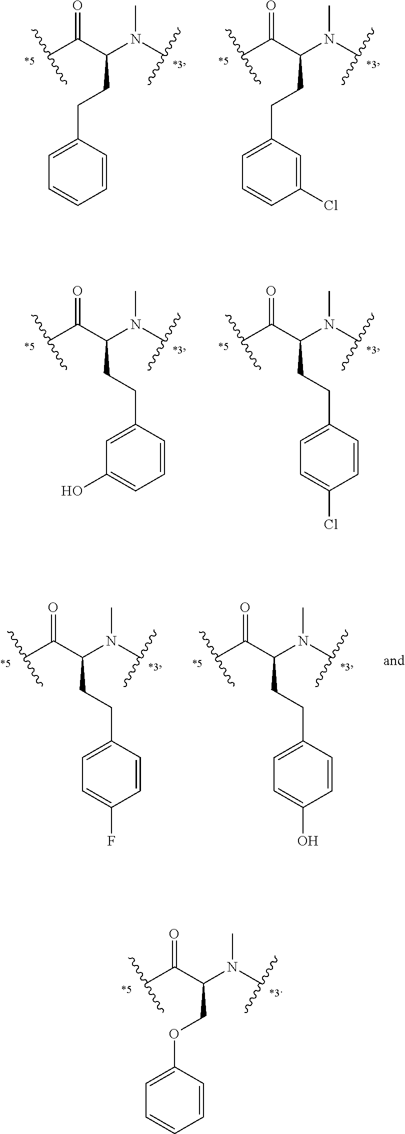

In some embodiments of the cyclic peptide (P), or compounds comprising cyclic peptide (P):

A⁴ is selected from:

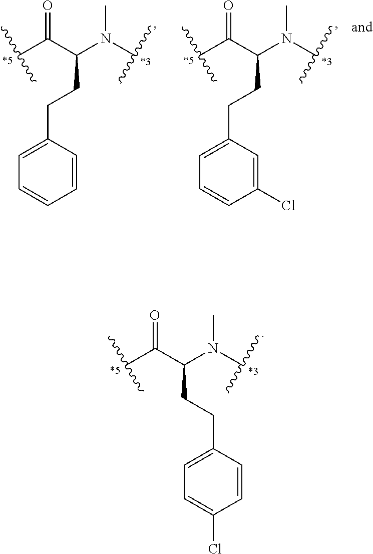

In some embodiments of the cyclic peptide (P), or compounds comprising cyclic peptide (P):
A⁵ is a moiety of formula (A5-I), formula (A5-II), or formula (A5-III):

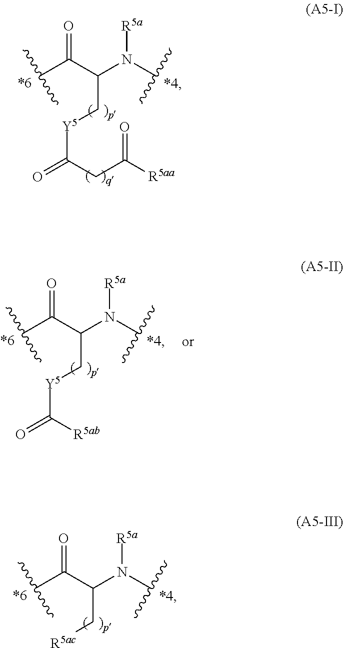

wherein:
Y⁵ is selected from O and NH;
R^{5a} is selected from H and C₁₋₃-alkyl;
R^{5aa} is selected from H, —OH, and —OC₁₋₆-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
R^{5ac} is selected from H, —NH₂, —NHC₁₋₆-alkyl, —N(C₁₋₆-alkyl)₂, and —OH; and
p′ and q′ are each independently 1, 2, 3, 4, 5, or 6.

In some embodiments of the cyclic peptide (P), or compounds comprising cyclic peptide (P):

A⁵ is selected from:

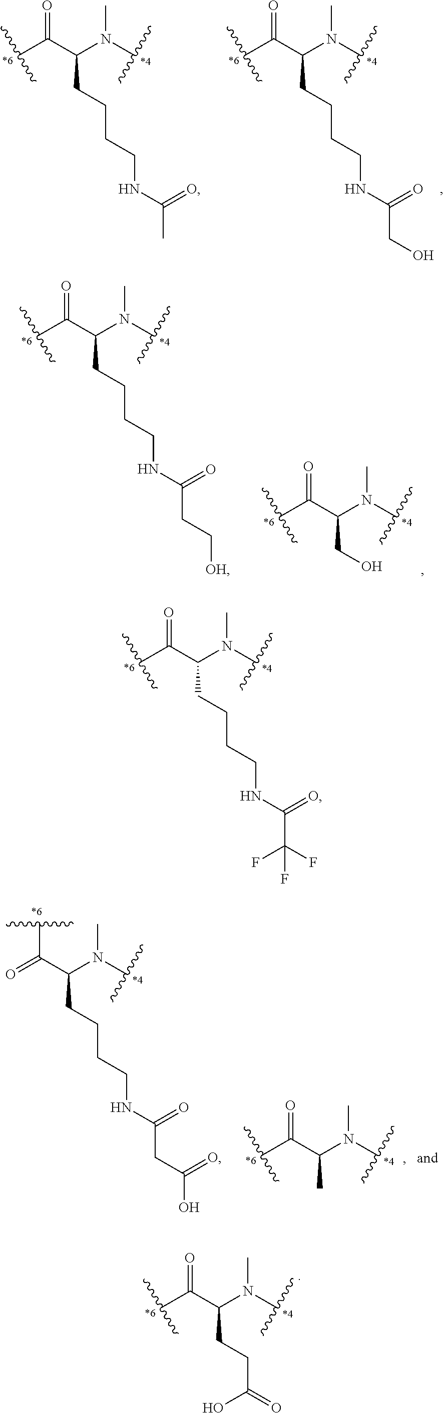

In some embodiments of the cyclic peptide (P), or compounds comprising cyclic peptide (P):

A⁵ is selected from:

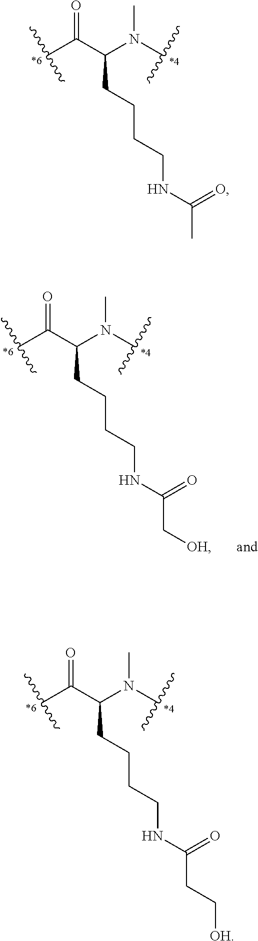 [SMILES: *C(=O)[C@H](CCCCNC(C)=O)N(*)C ; *C(=O)[C@H](CCCCNC(=O)CO)N(*)C ; "and" ; *C(=O)[C@H](CCCCNC(=O)CCO)N(*)C]

In some embodiments of the cyclic peptide (P), or compounds comprising cyclic peptide (P):

R^{6c} is selected from C₁₋₆-alkyl and 9-membered heteroaryl, wherein the C₁₋₆-alkyl and 9-membered heteroaryl of R⁶ are each optionally substituted with 1 or 2 substituents independently selected from C₁₋₆-alkyl, —CN, halo, —NH₂, —OH, —OC₁₋₆-alkyl, and —OC(O)—C₁-6-alkyl.

In some embodiments of the cyclic peptide (P), or compounds comprising cyclic peptide (P):

the 9-membered heteroaryl of R⁶ is a bicyclic 9-membered heteroaryl.

In some embodiments of the cyclic peptide (P), or compounds comprising cyclic peptide (P):
A⁶ is a moiety of formula (A6-I), formula (A6-II), or formula (A6-III):

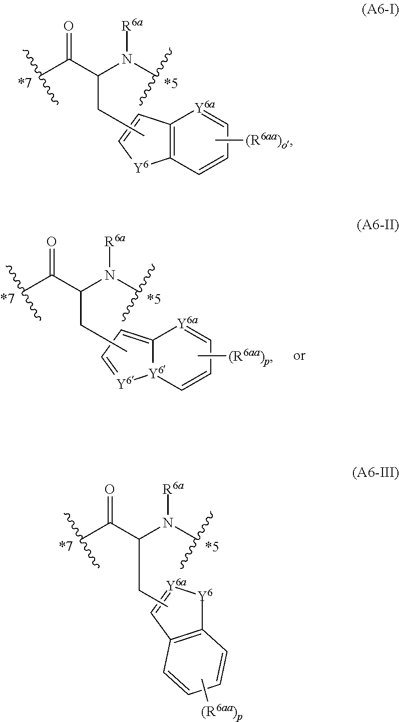

wherein:
Y⁶ is selected from NH and CH₂;
Y^{6a} and Y^{6′} are each independently selected from N and CH, provided that at least one of Y⁶ and Y^{6a} is selected from N and NH and provided that at least one of Y^{6′} and Y^{6a} is N;
R^{6a} is selected from H and C₁₋₃-alkyl;
each R^{6aa} is selected from C₁₋₆-alkyl, —CN, halo, —NH₂, —OH, —OC₁₋₆-alkyl, and —OC(O)—C₁₋₆-alkyl; and
o′ and p are each independently 0, 1, or 2.

In some embodiments of the cyclic peptide (P), or compounds comprising cyclic peptide (P):
A⁶ is a moiety of formula (A6-Ia), formula (A6-IIa), or formula (A6-IIIa):

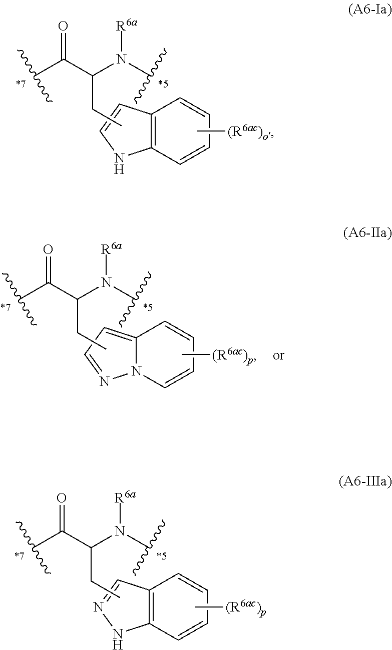

wherein:
R^{6a} is selected from H and C₁₋₃-alkyl;
each R^{6ac} is selected from C₁₋₆-alkyl, halo, —OH, —OC₁₋₆-alkyl, and —OC(O)—C₁₋₆-alkyl; and
o′ and p are each independently 0, 1, or 2.

In some embodiments of the cyclic peptide (P), or compounds comprising cyclic peptide (P):

A⁶ is selected from:

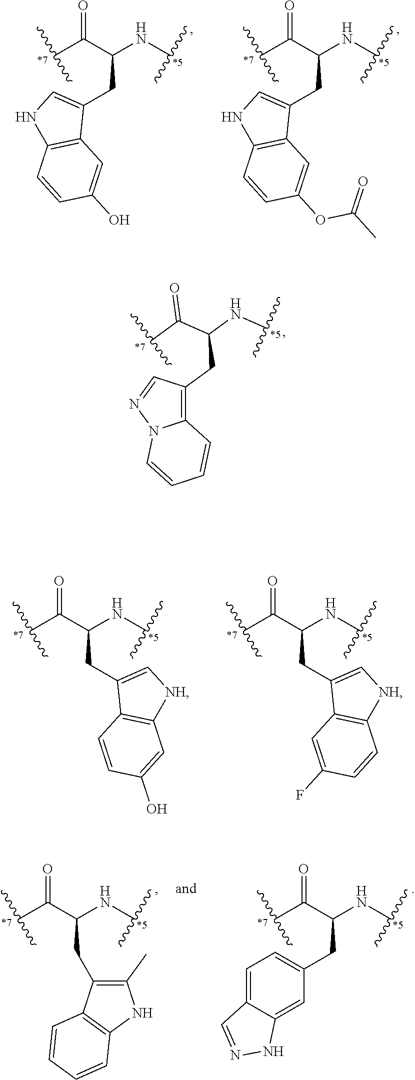

In some embodiments of the cyclic peptide (P), or compounds comprising cyclic peptide (P):

A⁶ is selected from:

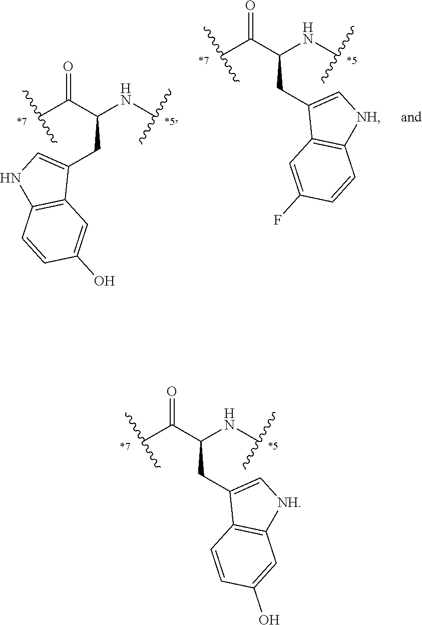 [SMILES: *N[C@@H](Cc1c[nH]c2ccc(O)cc12)C(*)=O ; *N[C@@H](Cc1c[nH]c2ccc(F)cc12)C(*)=O ; *N[C@@H](Cc1c[nH]c2cc(O)ccc12)C(*)=O]

In some embodiments of the cyclic peptide (P), or compounds comprising cyclic peptide (P):

R^{7c} is selected from C₁₋₆-alkyl and 9-membered heteroaryl, wherein the C₁₋₆-alkyl and 9-membered heteroaryl of R^{7c} are each optionally substituted with 1 or 2 substituents independently selected from —CN, halo, —NH₂, and —OH;

In some embodiments of the cyclic peptide (P), or compounds comprising cyclic peptide (P):

the 9-membered heteroaryl of R⁷ is a bicyclic 9-membered heteroaryl.

In some embodiments of the cyclic peptide (P), or compounds comprising cyclic peptide (P):
A⁷ is a moiety of formula (A7-I) or formula (A7-II):

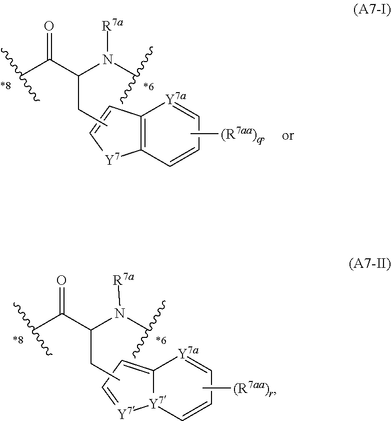

wherein:
Y⁷ is selected from NH and CH₂;
Y^{7a} and Y^{7′} are each independently selected from N and CH, provided that at least one of Y⁷ and Y^{7a} is selected from N and NH and provided that at least one of Y^{7′} and Y^{7a} is N;
R^{7a} is selected from H and C₁₋₃-alkyl;
each R^{7aa} is selected from C₁₋₃-alkyl, —CN, halo, —NH₂, and —OH; and
q and r are each independently 0, 1, or 2.

In some embodiments of the cyclic peptide (P), or compounds comprising cyclic peptide (P):

A⁷ is in the L configuration.

In some embodiments of the cyclic peptide (P), or compounds comprising cyclic peptide (P):
A⁷ is a moiety of formula (A7-Iaa):

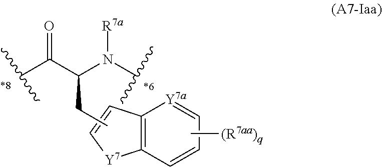

wherein:
Y⁷ is selected from NH and CH₂;
Y^{7a} is selected from N and CH, provided that at least one of Y⁷ and Y^{7a} is selected from N and NH;
R^{7a} is selected from H and C₁₋₃-alkyl;
each R^{7aa} is selected from C₁₋₃-alkyl, —CN, halo, —NH₂, and —OH; and
q is 0, 1, or 2.

In some embodiments of the cyclic peptide (P), or compounds comprising cyclic peptide (P):
A⁷ is a moiety of formula (A7-Ia) or formula (A7-IIa):

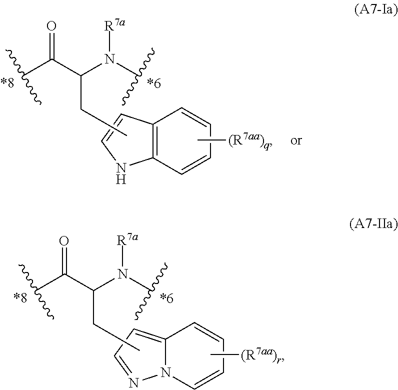

wherein:
R^{7a} is selected from H and C₁₋₃-alkyl;
each R^{7aa} is selected from C₁₋₃-alkyl, halo, —NH₂, and —OH; and
q and r are each independently 0, 1, or 2.

In some embodiments of the cyclic peptide (P), or compounds comprising cyclic peptide (P):
A⁷ is a moiety of formula (A7-Iaaa):

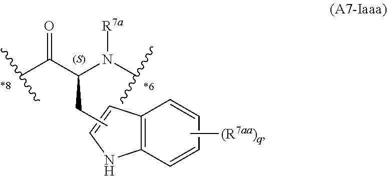

wherein:
R^{7a} is selected from H and C₁₋₃-alkyl;
each R^{7aa} is selected from C₁₋₃-alkyl, halo, —NH₂, and —OH; and
q is 0, 1, or 2.

In some embodiments of the cyclic peptide (P), or compounds comprising cyclic peptide (P):

A⁷ is selected from:

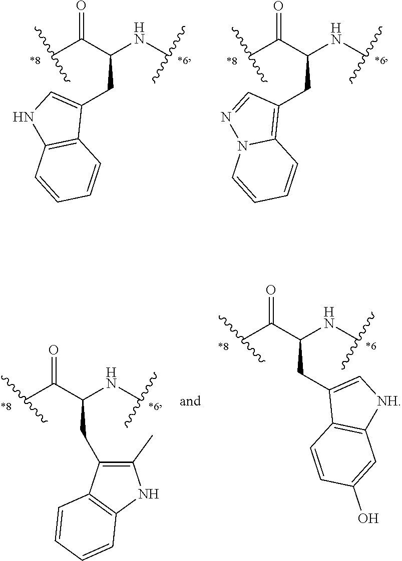 [SMILES: *N[C@@H](Cc1c[nH]c2ccccc12)C(*)=O ; *N[C@@H](Cc1cnn2ccccc12)C(*)=O ; *N[C@@H](Cc1c(C)[nH]c2ccccc12)C(*)=O ; *N[C@@H](Cc1c[nH]c2cc(O)ccc12)C(*)=O]

In some embodiments of the cyclic peptide (P), or compounds comprising cyclic peptide (P):

A⁷ is selected from:

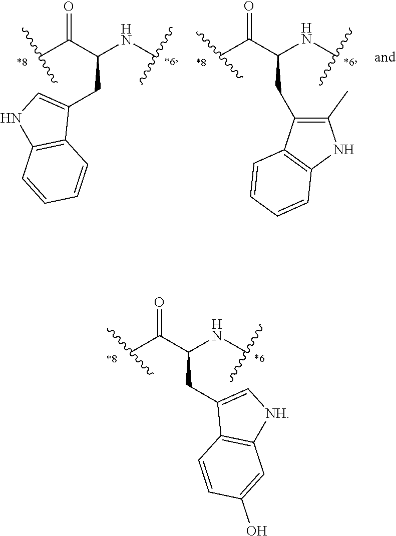 [SMILES: *N[C@@H](Cc1c[nH]c2ccccc12)C(*)=O ; *N[C@@H](Cc1c(C)[nH]c2ccccc12)C(*)=O ; *N[C@@H](Cc1c[nH]c2cc(O)ccc12)C(*)=O]

In some embodiments of the cyclic peptide (P), or compounds comprising cyclic peptide (P):
A⁸ is a moiety of formula (A8-I) or formula (A8-II):

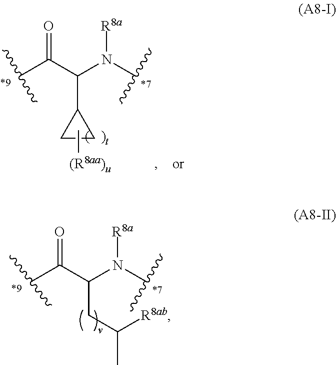

wherein:
R^{8a} is selected from H and C₁₋₃-alkyl;
R^{8aa} is selected from —CN, halo, —NH₂, and —OH;
R^{8ab} is selected from H, —CN, halo, —NH₂, and —OH;
t is 1 or 2;
u is 0, 1, or 2; and
v is 0, 1, 2, 3, or 4.

In some embodiments of the cyclic peptide (P), or compounds comprising cyclic peptide (P):

A⁸ is selected from:

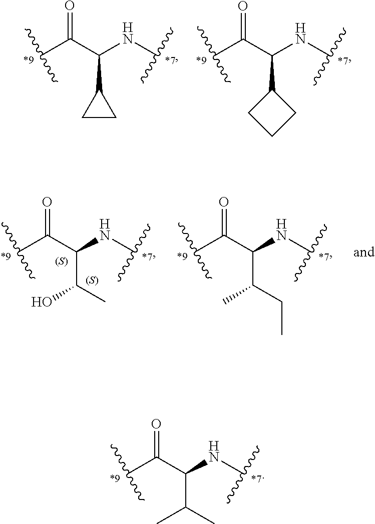

In some embodiments of the cyclic peptide (P), or compounds comprising cyclic peptide (P):

A⁸ is selected from:

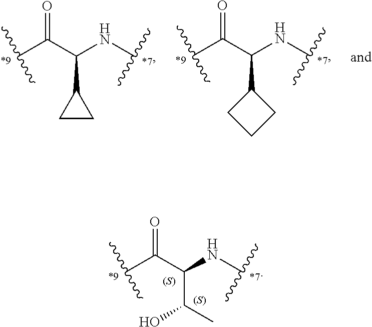

In some embodiments of the cyclic peptide (P), or compounds comprising cyclic peptide (P):
A⁹ is a moiety of formula (A9-I) or formula (A9-II):

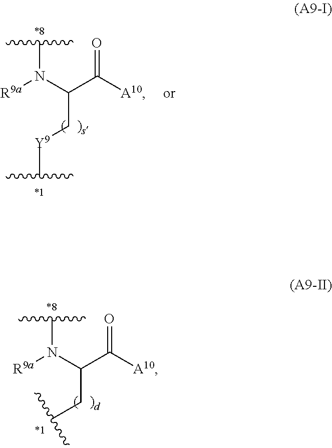

wherein:
Y⁹ is selected from C(O), NH, NC₁₋₆-alkyl, and S;
R^{9a} is selected from H and C₁₋₃-alkyl; and
s′ is 1 or 2; and
d is 1 or 2.

In some embodiments of the cyclic peptide (P), or compounds comprising cyclic peptide (P):

A⁹ is selected from:

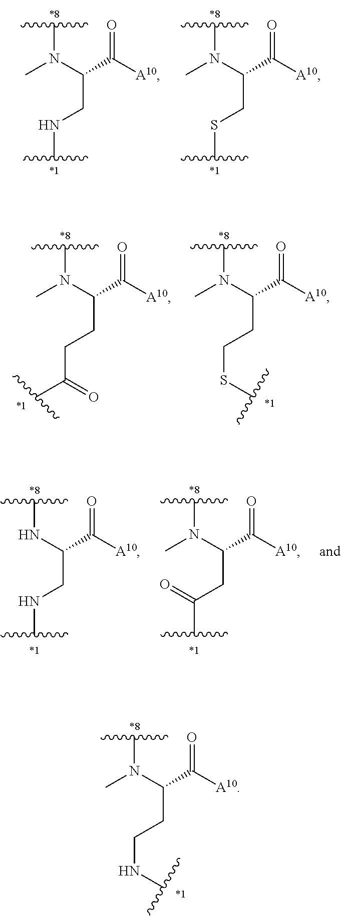

In some embodiments of the cyclic peptide (P), or compounds comprising cyclic peptide (P):

A⁹ is selected from:

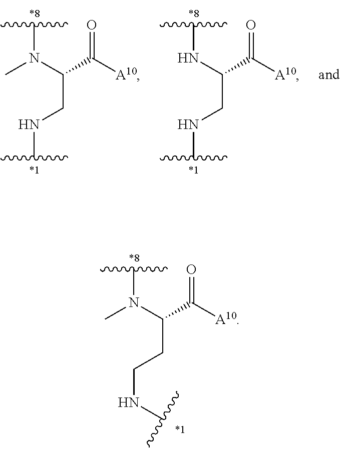

In some embodiments of the cyclic peptide (P), or compounds comprising cyclic peptide (P):

A¹⁰ is a moiety of formula (A10-I), formula (A10-II), formula (A10-III), or formula (A¹⁰-IV):

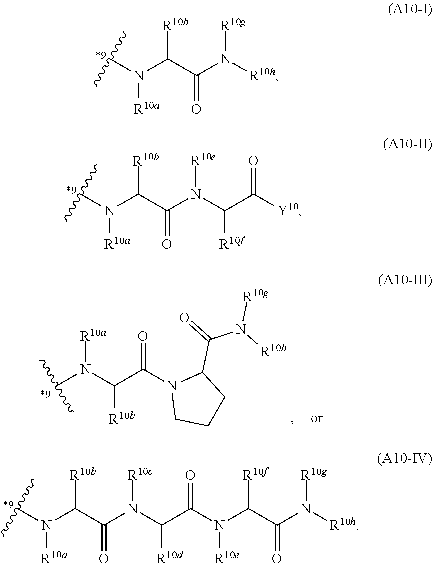

In some embodiments of the cyclic peptide (P), or compounds comprising cyclic peptide (P):
A¹⁰ is a moiety of formula (A10-Ia) or formula (A10-Ib):

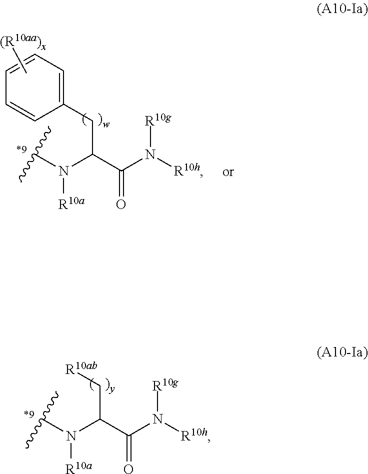

wherein:
R^{10a} is selected from H and C₁₋₃-alkyl;
R^{10g} and R^{10h} are each independently selected from H and C₁₋₆-alkyl;
each R₁₀ₐₐ is independently selected from C₁₋₆-alkyl, halo, —NH₂, —N₃, —OH, and —OC₁-6-alkyl;
R^{10ab} is selected from H, halo, —NH₂, —N₃, —OH, and —OC₁₋₆-alkyl;
w and y are each independently 1, 2, 3, 4, 5, or 6; and
x is 0, 1, or 2.

In some embodiments of the cyclic peptide (P), or compounds comprising cyclic peptide (P):
A¹⁰ is a moiety of formula (A10-IIa), formula (A10-IIb), or formula (A10-IIc):

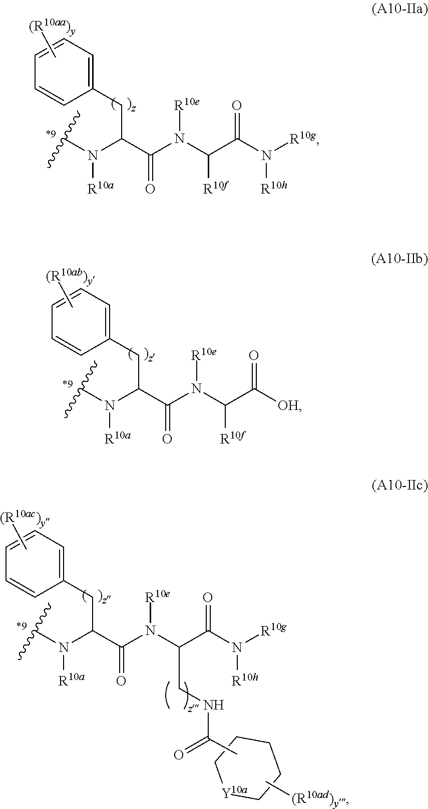

wherein:
each Y^{10a} is independently selected from N, NH, and CH₂, provided that at least one Y^{10a} is selected from N and NH;
R^{10a} and R^{10e} are each independently selected from H and C₁₋₃-alkyl;
R^{10g} and R^{10h} are each independently selected from H and C₁₋₆-alkyl;
R^{10f} is selected from H and C₁₋₆-alkyl, wherein the C₁₋₆-alkyl of R^{10f} is optionally substituted with 1 or 2 substituents independently selected from halo, —NH₂, —N₃, —OH, and —OC₁₋₆-alkyl;
R^{10aa}, R^{10ab}, and R^{10ac} are each independently selected from C₁₋₆-alkyl, halo, —NH₂, —N₃, —OH, and —OC₁₋₆-alkyl;
each R^{10ad} is independently selected from C₁₋₆-alkyl, halo, —NH₂, —N₃, —OH, —OC₁₋₆-alkyl, —C(O)NH₂, —C(O)OH, and —C₁₋₆-alkyl-C(O)OH;
y, y′, y″, and y′″ are each independently 0, 1, or 2; and
z, z′, z″, and z″″ are each independently 1, 2, 3, 4, 5, or 6.

In some embodiments of the cyclic peptide (P), or compounds comprising cyclic peptide (P):
A¹⁰ is a moiety of formula (A10-IIIa):

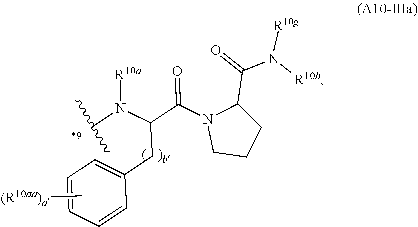

wherein:
R^{10a} is selected from H and C₁₋₃-alkyl;
R^{10g} and R^{10h} are each independently selected from H and C₁₋₆-alkyl;
R^{10aa} is selected from C₁₋₆-alkyl, halo, —NH₂, —N₃, —OH, and —OC₁₋₆-alkyl;
a′ is 0, 1, or 2; and
b′ is 1, 2, 3, 4, 5, or 6.

In some embodiments of the cyclic peptide (P), or compounds comprising cyclic peptide (P):
A¹⁰ is a moiety of formula (A10-IVa):

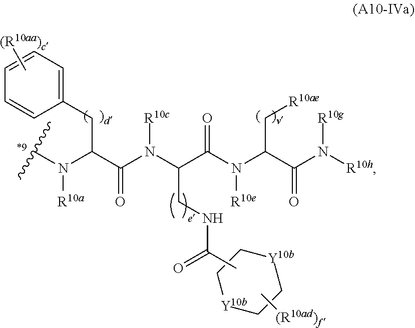

wherein:
each Y^{10b} is independently selected from N, NH, and CH₂, provided that at least one Y^{10b} is selected from N and NH;
R^{10a}, R^{10c}, and R^{10e} are each independently selected from H and C₁₋₃-alkyl;
R^{10g} and R^{10h} are each independently selected from H and C₁₋₆-alkyl;
R^{10aa} is selected from C₁₋₆-alkyl, halo, —NH₂, —N₃, —OH, —OC₁₋₆-alkyl;
each R^{10ad} is independently selected from C₁₋₆-alkyl, halo, —NH₂, —N₃, —OH, —OC₁₋₆-alkyl, —C(O)NH₂, —C(O)OH, and —C₁₋₆-alkyl-C(O)OH;
R^{10ae} is selected from H, halo, —NH₂, —N₃, —OH, and —OC₁₋₆-alkyl;
c′ and f′ are each independently 0, 1, or 2; and
d′, e′, and v′ are each independently 1, 2, 3, 4, 5, or 6.

In some embodiments of the cyclic peptide (P), or compounds comprising cyclic peptide (P):

A¹⁰ is selected from:

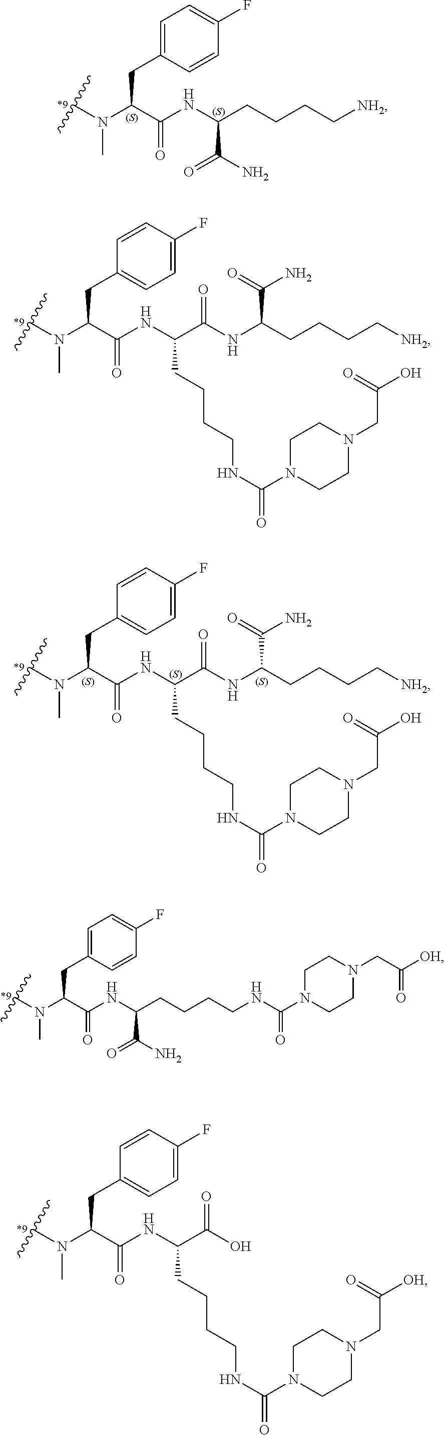

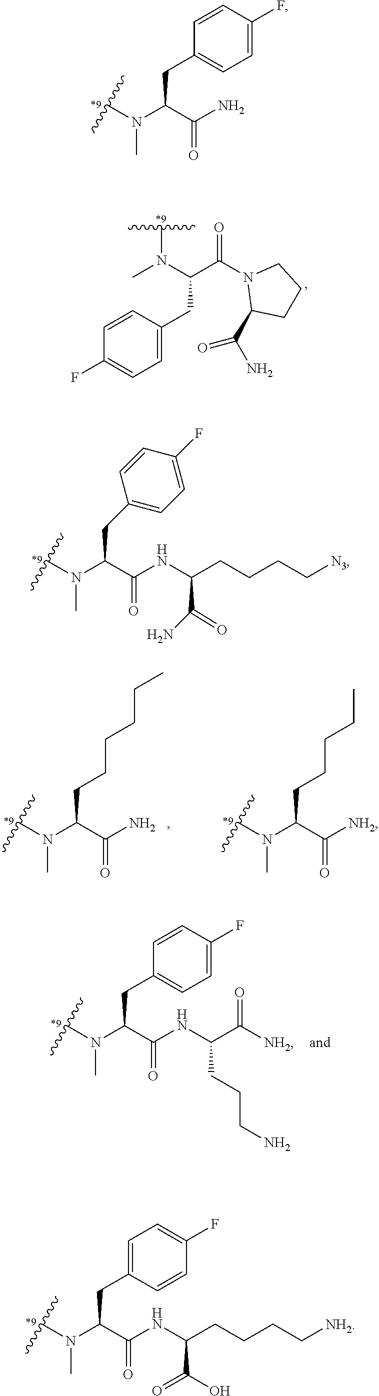

In some embodiments of the cyclic peptide (P), or compounds comprising cyclic peptide (P):

A¹⁰ is selected from:

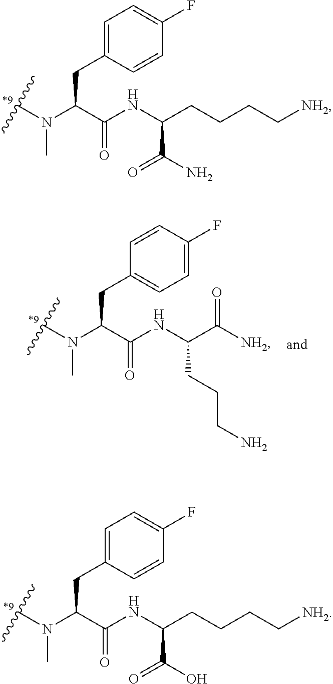 [SMILES: *N(C)[C@@H](Cc1ccc(F)cc1)C(=O)N[C@@H](CCCCN)C(N)=O ; *N(C)[C@@H](Cc1ccc(F)cc1)C(=O)N[C@@H](CCCN)C(N)=O ; *N(C)[C@@H](Cc1ccc(F)cc1)C(=O)N[C@@H](CCCCN)C(=O)O]

In some embodiments of the cyclic peptide (P), or compounds comprising cyclic peptide (P), A¹⁰ is substituted with one biotin tag.

In certain embodiments, the cyclic peptide (P) is of Formula (P-I):

*^{α}-A¹-Tyr-Ser-A⁴-A⁵-(W(5OH))-Trp-A⁸-(NMeDap)*^{β}-A¹⁰-, wherein
*^{α} indicates the point of attachment of A¹ to NMeDap;
*^{β} indicates the point of attachment of NMeDap to A¹, wherein A¹ and NMeDap are attached so Formula (P-I) is a cyclized (cyclic) peptide (P);
A¹ is: NAc-hE, hE, NAc-E, H-GlutarA, or NAc-Asp, each of which is optionally substituted with a PEG chain;
A⁴ is NMehF, NMeAhp, NMeAoc, or NMehF(3Cl);
A⁵ is: MeKAc, NMeS, NMe-K (TFA), MeK(COCH₂OH), or MeK(COEtOH);
A⁸ is: CproG or AlloT;
A¹⁰ is NMeF(4F), NMeAoc, or NMeAhp;
and A¹⁰ is bound to L¹, wherein L¹ is a bond or a linker (e.g., Orn or Lys), wherein L¹ is bound to M, and M is an imaging agent, chelating agent, radionuclide, or cytotoxic drug (e.g., M is a chelator optionally radiolabeled with a radionuclide).

In certain further embodiments, the cyclic peptide (P) of Formula (P-I) is of Formula (P-I-A):

*^{α}-A¹-Tyr-Ser-A⁴-A⁵-(W(5OH))-Trp-A⁸-(NMeDap)*^{β}-A¹⁰-, wherein
*^{α} indicates the point of attachment of A¹ to NMeDap;
*^{β} indicates the point of attachment of NMeDap to A¹, wherein A¹ and NMeDap are attached so Formula (P-I-A) is a cyclized peptide (P);
A¹ is: NAc-hE, hE, or NAc-E, each of which is optionally substituted with a PEG chain (e.g., mPEG12);
A⁴ is NMehF or NMehF (3Cl);
A⁵ is: MeKAc or NMeS;
A⁸ is: CproG or AlloT;
A¹⁰ is NMeF(4F);
and A¹⁰ is bound to L′, wherein L′ is a bond or a linker (e.g., Orn or Lys), wherein L¹ is bound to M, and M is a chelator (e.g., a chelator of Tables 3-7) optionally radiolabeled with a radionuclide (e.g., ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th).

In certain further embodiments, the cyclic peptide (P) of Formula (P-I) is of Formula (P-I-A-i):

*^{α}-A¹-Tyr-Ser-(NMehF)-A⁵-(W(5OH))-Trp-A⁸-(NMeDap)*^{β}—(NMeF(4F))—, wherein
*^{α} indicates the point of attachment of A¹ to NMeDap;
*^{β} indicates the point of attachment of NMeDap to A¹, wherein A¹ and NMeDap are attached so Formula (P-I-A-i) is a cyclized peptide (P);
A¹ is: NAc-hE or mPEG12-hE, wherein mPEG12-hE has the following structure:

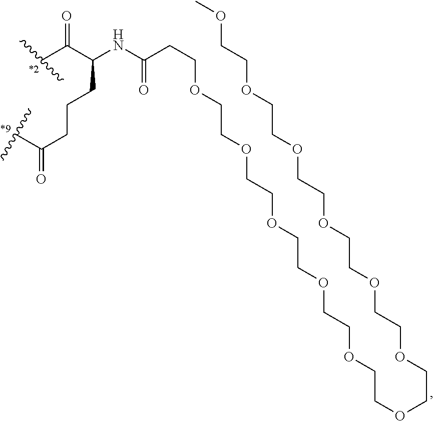 [SMILES: *C(=O)CCC[C@H](NC(=O)CCOCCOCCOCCOCCOCCOCCOCCOCCOCCOCCOCCOC)C(*)=O]

wherein *2 indicates the point of attachment to A² and *9 indicates the point of attachment to A⁹;
A⁵ is: MeKAc or NMeS;
A⁸ is: CproG or AlloT;
and NMeF(4F) is bound to L¹, wherein L¹ is a bond or Lys, wherein L¹ is bound to M, and M is a chelator selected from DOTA, NOTA, DOTAGA, and NODAGA, wherein M is optionally radiolabeled with a radionuclide (e.g., ¹⁷⁷Lu, ¹⁶¹Tb, ⁹⁰Y, ⁶⁷Cu, ²²⁵Ac, ²¹²Pb, ²¹¹At, or ²²⁷Th);
wherein NMeF(4F), L¹, and M together have a structure selected from

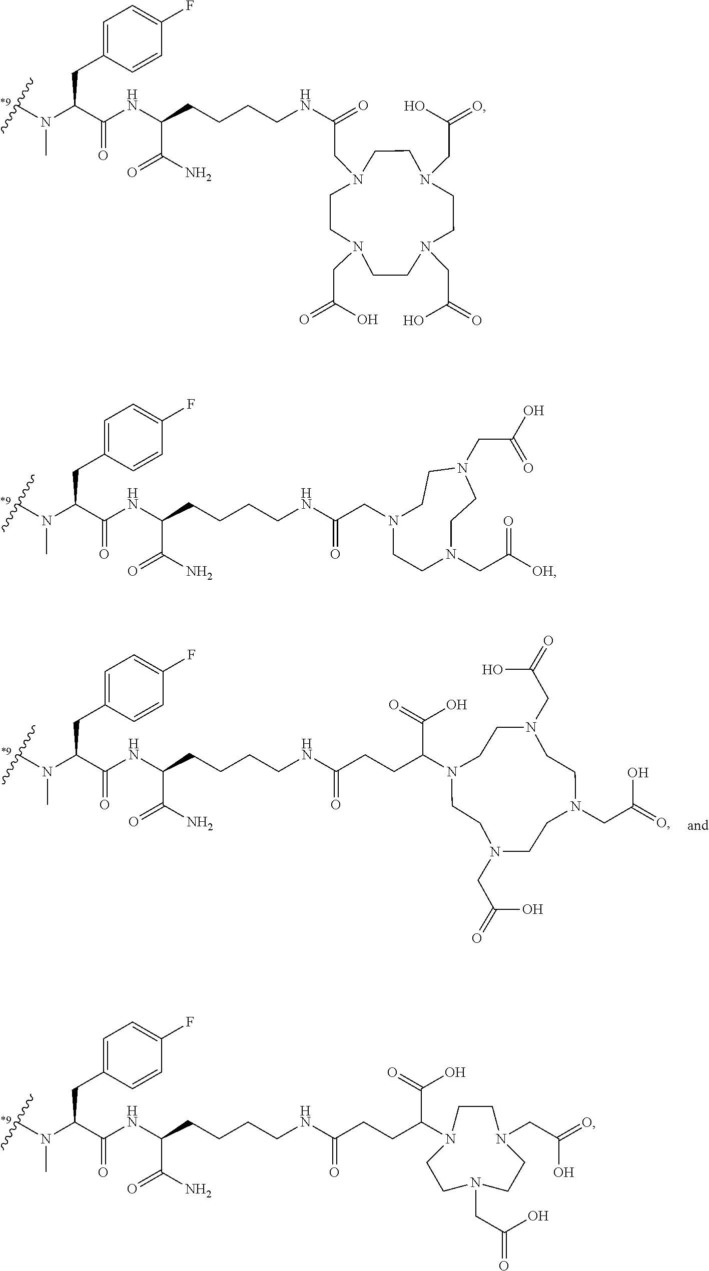 [SMILES: *N(C)[C@@H](Cc1ccc(F)cc1)C(=O)N[C@@H](CCCCNC(=O)CN1CCN(CC(=O)O)CCN(CC(=O)O)CCN(CC(=O)O)CC1)C(N)=O ; *N(C)[C@@H](Cc1ccc(F)cc1)C(=O)N[C@@H](CCCCNC(=O)CN1CCN(CC(=O)O)CCN(CC(=O)O)CC1)C(N)=O ; *N(C)[C@@H](Cc1ccc(F)cc1)C(=O)N[C@@H](CCCCNC(=O)CCC(C(=O)O)N1CCN(CC(=O)O)CCN(CC(=O)O)CCN(CC(=O)O)CC1)C(N)=O ; *N(C)[C@@H](Cc1ccc(F)cc1)C(=O)N[C@@H](CCCCNC(=O)CCC(C(=O)O)N1CCN(CC(=O)O)CCN(CC(=O)O)CC1)C(N)=O]

wherein:
*9 indicates the point of attachment to A⁹,
wherein M is optionally radiolabeled with a radionuclide.

In certain embodiments, the cyclic peptide (P) is a cyclic peptide selected from any one of Examples A¹ to A⁷⁷, or a pharmaceutically acceptable salt, solvate, stereoisomer, or tautomer thereof.

In certain embodiments, the cyclic peptide (P) is a cyclic peptide selected from any one of Examples A¹ to A⁷⁷, or a pharmaceutically acceptable salt, solvate, stereoisomer, or tautomer thereof. In certain embodiments, a compound of the present disclosure (e.g., ligand, for example, radioligand) is a compound of the table of Example III, or a pharmaceutically acceptable salt, solvate, stereoisomer, or tautomer thereof.

### Albumin Binders

The HER2-targeting cyclic peptide (P), or the compound comprising a cyclic peptide (P), of the present disclosure may further comprise an albumin binder. For example, an albumin binder may be attached to the cyclic peptide via a side chain of any one of A¹-A¹⁰. In one or more embodiments, an albumin binder is connected to the cyclic peptide via a side chain of A².

Any compound, peptide, polypeptide, or other moiety known in the art as being capable of binding albumin, i.e., having albumin binding affinity, may be connected to the cyclic peptide (or any of the compounds comprising said cyclic peptide) of the instant disclosure as an albumin binder. Addition of an albumin binder to the cyclic peptide may prolong the bioavailability of the disclosed HER2-targeting compounds, thereby enhancing tumor uptake. Exemplary albumin binders are described in Zorzi et al. Non-covalent albumin-binding ligands for extending the circulating half-life of small biotherapeutics. Med. Chem. Comm. 2019 Jun. 6; 10 (7): 1068-1081, Brandt et al. Mini-review: Targeted radiopharmaceuticals incorporating reversible, low molecular weight albumin binders. Nucl. Med. Biol. 2019 March; 70:46-52, and Lau et al. Bench to Bedside: Albumin Binders for Improved Cancer Radioligand Therapies. Bioconjug. Chem. 2019 Mar. 20; 30 (3): 487-502. 10.1021/acs.bioconjchem.8b00919. Epub 2019 Jan. 23. PMID: 30616340, which are each incorporated herein by reference in its entirety.

In one or more embodiments, the cyclic peptide, or any of the compounds disclosed herein comprising said cyclic peptide, is substituted with an albumin binder selected from fatty acids and derivatives thereof. Such fatty acids and derivatives thereof include, e.g.,

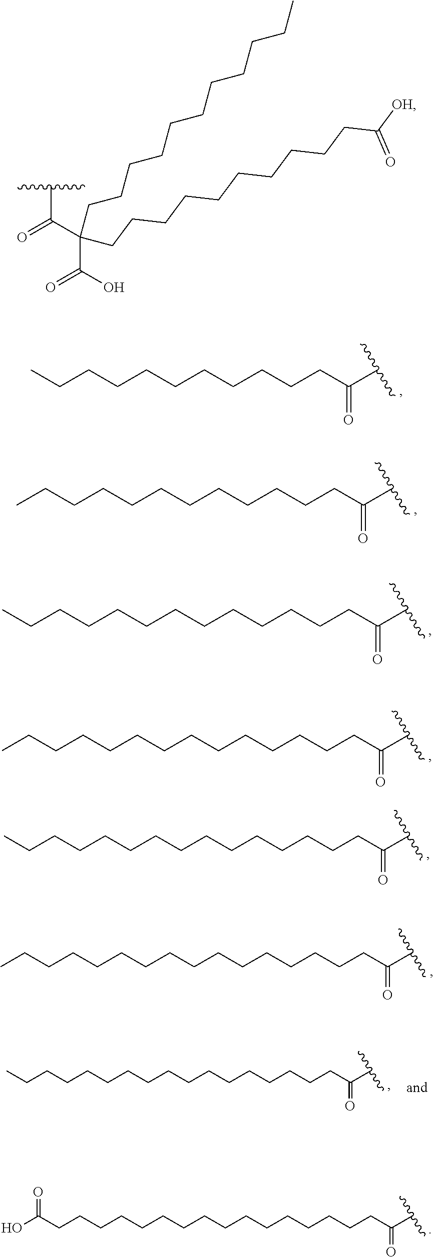

In one or more embodiments, the cyclic peptide, or any of the compounds disclosed herein comprising said cyclic peptide, is substituted with an albumin binder selected from:

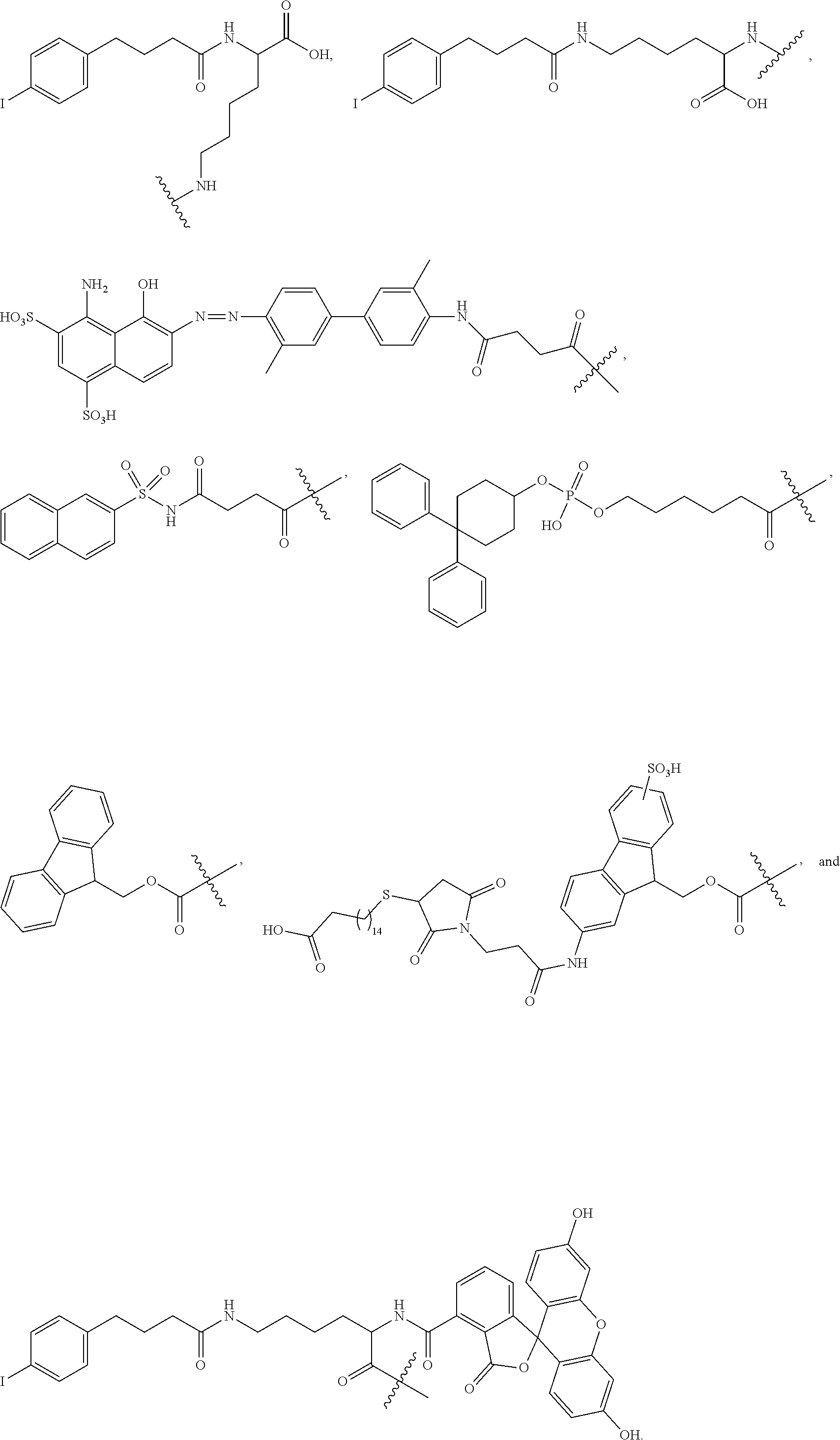

In various embodiments, the albumin binder is 4-(p-iodophenyl) butyric acid or a derivative thereof, such as, for example:

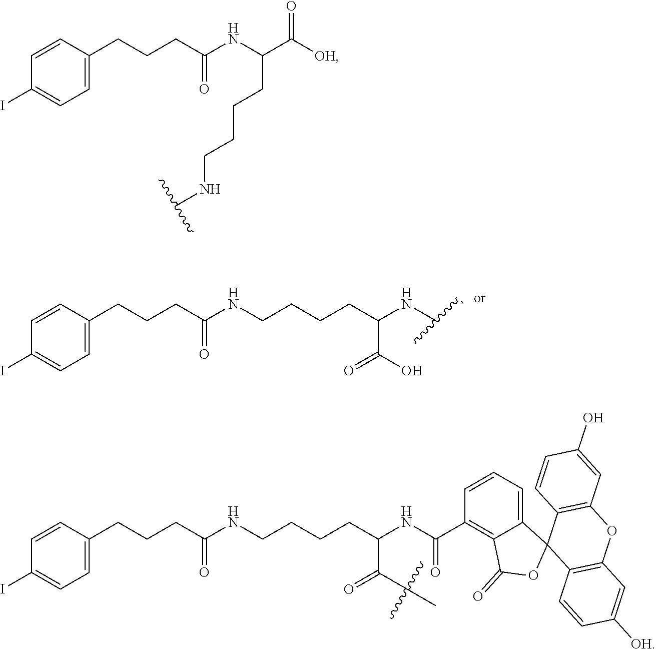 [SMILES: *NCCCCC(NC(=O)CCCc1ccc(I)cc1)C(=O)O ; *NC(CCCCNC(=O)CCCc1ccc(I)cc1)C(=O)O ; *C(=O)C(CCCCNC(=O)CCCc1ccc(I)cc1)NC(=O)c1cccc2c1C(=O)OC21c2ccc(O)cc2Oc2cc(O)ccc21]

In some embodiments, the albumin binder is:

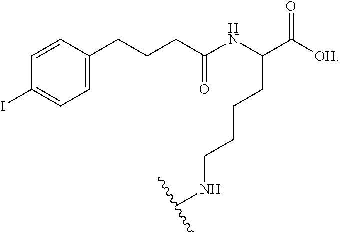 [SMILES: *NCCCCC(NC(=O)CCCc1ccc(I)cc1)C(=O)O]

In some embodiments, the albumin binder is:

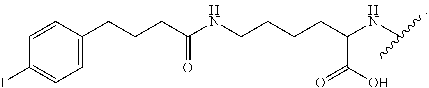 [SMILES: *NC(CCCCNC(=O)CCCc1ccc(I)cc1)C(=O)O]

In some embodiments, the albumin binder is Evans blue, or a derivative thereof. In some embodiments, the albumin binder is:

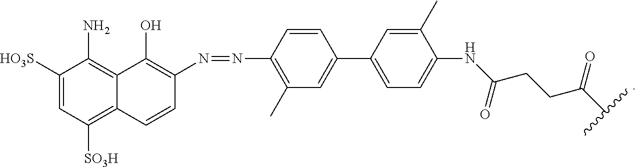 [SMILES: *C(=O)CCC(=O)Nc1ccc(-c2ccc(N=Nc3ccc4c(S(=O)(=O)O)cc(S(=O)(=O)O)c(N)c4c3O)c(C)c2)cc1C]

In some embodiments when the cyclic peptide, or compound comprising said cyclic peptide, is substituted with an albumin binder cyclic peptide, said cyclic peptide, is a compound that is Example G1, or a pharmaceutically acceptable salt or solvate thereof.

### L¹ (e.g., of Compounds of Formulae (I), (Ia), (Ib), (Ic), and (Id))

The compounds disclosed herein (e.g., compounds of Formulae (I), (Ia), (Ib), (Ic), and (Id) comprise L¹ between P and M. In some embodiments, L¹ is a bond (i.e., a bond between P and M). In some embodiments, L¹ is a linker. As used herein, the term “linker” means a structural component that connects two parts of a compound. Linkers can comprise an atom such as oxygen or sulfur, a unit such as NR, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylheterocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylheteroaryl. In any of these structural components, one or more methylene groups can be interrupted by O, S, S(O), SO₂, N(R′), C(O), substituted or unsubstituted aryl, substituted or unsubstituted heteroaryl, substituted or unsubstituted heterocyclic; where R′ is hydrogen, acyl, aliphatic or substituted aliphatic. In some embodiments, the linker L is one to about twenty-four atoms, one to about twelve atoms, one to about eight atoms, one to about six atoms, or four to six atoms.

The linker may be attached to any amino acid site of the cyclic peptide (P) to form a linkage between P and M. For example, the linker may be attached to the HER2-targeting compound via any one of A¹-A¹⁰. In some embodiments, the linker is attached to a side chain of any one of A¹-A¹⁰. In other embodiments, the linker is attached to a backbone nitrogen of any one of A¹-A¹⁰. In some embodiments, the linker is attached to the side chain or backbone nitrogen of A¹. In some embodiments, the linker is attached to the side chain or backbone nitrogen of A². In some embodiments, the linker is attached to the side chain or backbone nitrogen of A³. In some embodiments, the linker is attached to the side chain or backbone nitrogen of A⁴. In some embodiments, the linker is attached to the side chain or backbone nitrogen of A⁵. In some embodiments, the linker is attached to the side chain or backbone nitrogen of A⁶. In some embodiments, the linker is attached to the side chain or backbone nitrogen of A⁷. In some embodiments, the linker is attached to the side chain or backbone nitrogen of A⁸. In some embodiments, the linker is attached to the side chain or backbone nitrogen of A⁹. In some embodiments, the linker is attached to the side chain or backbone nitrogen of A¹⁰.

Analogously, the linker may be attached to any site capable of forming a covalent attachment on the chelator to form a linkage between P and M. For example, the linker may be attached to the chelator via a heteroatom, the functional group of a functionalized heteroatom, or an alkyl, cycloalkyl, or aryl group separating the heteroatoms of the chelator. In some embodiments, the linker is attached to a heteroatom of the chelator. In other embodiments, the linker is attached to the functional group of a functionalized heteroatom of the chelator. In yet other embodiments, the linker is attached to an alkyl, cycloalkyl, aryl, or heteroaryl group separating the heteroatoms of the chelator. In some embodiments, the linker is attached to an alkyl group separating the heteroatoms of the chelator. In some embodiments, the linker is attached to a cycloalkyl group separating the heteroatoms of the chelator. In some embodiments, the linker is attached to an aryl group separating the heteroatoms of the chelator.

In some embodiments, the linker is a covalent bond. Alternatively, in some embodiments, the linker is a divalent moiety other than a covalent bond. In embodiments in which the linker is a divalent moiety other than a bond, the linker comprises one or more functional groups capable of forming a covalent attachment with the cyclic peptide (P) or chelator (M). Suitable functional groups include, but are not limited to, amine, azide, amide, carboxylic acid, hydroxy, alkoxy, nitrile, alkene, and alkyne functional groups.

L¹ may be any linker known in the art. In some embodiments, the linker includes a group such as, for example, an amino acid, or derivative thereof, CH₂, CH₂CH₂, cycloalkylene, alkylene, arylene, alkylarylene, heteroarylene, heterocycloalkylene, (CR⁴R⁵)ₚO(CR⁴R⁵)_{q}, (CR⁴R⁵)ₚN(CR⁴R⁵)_{q}, (CR⁴R⁵)ₚS(CR⁴R⁵)_{q}, wherein cycloalkylene, alkylene, arylene, alkylarylene, heteroarylene, and heterocycloalkylene are optionally substituted with 1, 2, or 3 substituents independently selected from halo, C₁₋₆ alkyl, C₂₋₆ alkenyl, C₂₋₆ alkynyl, C₁₋₆ haloalkyl, halosulfanyl, CN, NO₂, N₃, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NR^{g}) NR^{c}R^{d}, NR^{c}C(═NR^{g}) NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NRCS(O)₂R^{b}, and S(O)₂NR^{c}R^{d},
wherein R⁴ and R⁵ are independently selected from H, halo, OH, C₁₋₆ alkyl, C₂₋₆ alkenyl, C₂₋₆ alkynyl, C₁₋₆ alkoxy, alkoxyalkyl, cyanoalkyl, heterocycloalkyl, cycloalkyl, C₁₋₆ haloalkyl, CN, and NO₂;
R^{a}, R^{b}, R^{c}, and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from —OH, —CN, amino, halo, C₁₋₆ alkyl, C₁₋₆ alkoxy, C₁₋₆ haloalkyl, and C₁₋₆ haloalkoxy
R^{e}, R^{f}, and R^{g} are each independently selected from the group consisting of H and C₁₋₁₀ alkyl;
p is 0, 1, 2, 3, 4, 5, or 6; and
q is 0, 1, 2, 3, 4, 5, or 6.

In various embodiments, at least one L¹ is bound to any-NH₂ or any C(O) of the cyclized peptide.

In various embodiments, at least one L¹ is bound to any-NH₂ or C(O) of A¹, A³, or A¹⁰.

In various embodiments, at least one L¹ is bound to any-NH₂ or any C(O) of A¹ or A¹⁰.

In various embodiments, L¹ is a linker of formula (L¹-I) or formula (L¹-II):

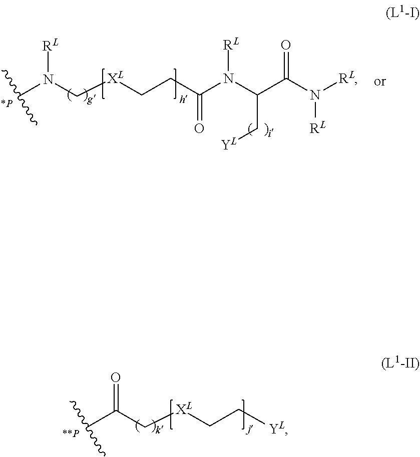

wherein:
X^{L} is selected from CH₂, N(R^{L}), and O;
Y^{L} is selected from H, —OH, —OC₁₋₆-alkyl, and —N(R^{L}) 2;
each R^{L} is independently selected from H and C₁₋₆-alkyl;
h′ and j′ are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
g′ and k′ are each independently 0, 1, 2, 3, or 4;
i′ is 0, 1, 2, 3, 4, 5, or 6; and
wherein *P indicates the point of attachment to a C(O) on A¹ or A¹⁰ and **P indicates the point of attachment to a —NH₂ on A¹ or A¹⁰.

In various embodiments, A¹⁰ and L¹, together, have the structure:

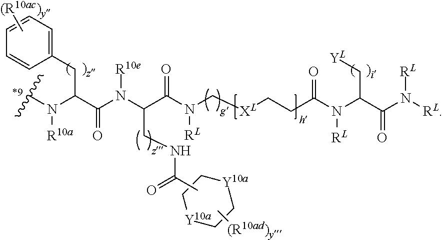

In various embodiments, A¹⁰ and L¹, together, have the structure:

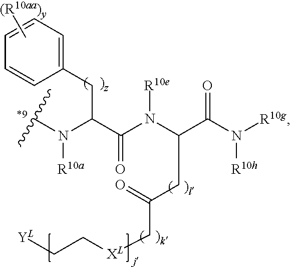

wherein:
l′ is 1, 2, 3, 4, 5, or 6.

In various embodiments, A¹ and L¹, together, have the structure:

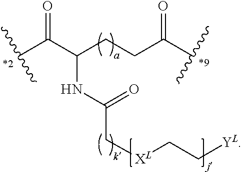

In various embodiments, L′ is a bond.

### M (e.g., of Compounds of Formulae (I), (Ia), (Ib), (Ic), and (Id))

As disclosed herein, M can be an imaging agent, a chelating agent optionally radiolabeled with a radionuclide, or a radionuclide. In some embodiments, M is an imaging agent. In some embodiments, M is a chelator. In some embodiments, M is a chelator radiolabeled with a radionuclide. In some embodiments, M is a radionuclide. Any M may be connected to the cyclic peptide via any L¹ described above (whether L′ is a linker or a bond). The skilled artisan would be able to determine suitable combinations of M and L¹ based on the present disclosure, or suitable substitution of the cyclic peptide directly with M in the case where L′ is a bond.

### Imaging Agents

In one or more embodiments of the HER2-targeting compounds described herein, M is an imaging agent. HER2-targeting compounds that include an imaging agent may be useful for in vitro and/or in vivo visualization of HER2-related disease progression or response after receiving the instantly disclosed compounds. In some embodiments, the imaging agent is a non-radioactive (or non-radionuclide) imaging agent. In some embodiments, the imaging agent is a fluorescent imaging agent.

Suitable imaging agents that may be connected to the cyclic peptide include Cy dyes, Culfo Cy dyes, Alexa Fluor dyes, Dylight dyes, FluoProbes dyes, Seta dyes, IRIS dyes, and other dyes that can be used interchangeably with Cy dyes in most biochemical imaging applications. For example, the compounds of the instant disclosure may be connected to imaging, but not limited to, Cy2, Cy3, Cy3.5, Cy5, Cy5.5, Cy7, Cy7.5, Sulfo Cy2, Sulfo Cy3, Sulfo Cy3.5, Sulfo Cy5, Sulfo Cy5.5, Sulfo Cy7, Sulfo Cy7.5, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Dylight 350, Dylight 405, Dylight 488, Dylight 550, Dylight 594, Dylight 633, Dylight 650, Dylight 680, Fluorprobes 390, Fluorprobes 488, Fluorprobes 532, Fluorprobes 547H, Fluorprobes 594, Fluorprobes 647H, Fluorprobes 682, Seta 375 NHS, Seta 400 NHS, Seta 405 NHS, Seta 475 Maleimide, Seta 470 NHS, Seta 555 Azide, Seta 555 DBCO, Seta 555 NHS, Seta 632 Maleimide, Seta 632 NHS, Seta 633 Azide, Seat 633 NHS, Seta 635 pH di-NHS, Seta 640 pH di-NHS, Seta 646, Maleimide, Seta 646 NHS, Seta 650 Azide, Seta 650 Maleimide, IRIS 2, IRIS 3, IRIS, 3.5, IRIS 5, IRIS 5.5, IRIS 7, IRIS 2 NHS Active, IRIS 3 NHS Active, IRIS 3.5 NHS Active, IRIS 5 NHS Active, IRIS 5.5 NHS Active, and IRIS 7 NHS Active.

Additional exemplary imaging agents that may be incorporated into a compound of the present disclosure includes those described in Elmes, R. Bioreductive fluorescent imaging agents: applications in tumor hypoxia. Chem. Commun. 2016, 52, 8935, and Schouw et al. Targeted optical fluorescence imaging: a meta-narrative review and future perspectives. Eur. J. Nucl. Med. Mol. Imaging. 2021 December; 48 (13): 4272-4292.

In some embodiments, the imaging agent is selected from Cy2, Cy3, Cy3.5, Cy5, Cy5.5, Cy7, Cy7.5, Sulfo Cy2, Sulfo Cy3, Sulfo Cy3.5, Sulfo Cy5, Sulfo Cy5.5, Sulfo Cy7, and Sulfo Cy7.5. In some embodiments, the imaging agent is selected from Cy3, Cy3.5, Cy5, Cy5.5, Cy7, Sulfo Cy3, Sulfo Cy3.5, Sulfo Cy5, Sulfo Cy5.5, and Sulfo Cy7. In some embodiments, the imaging agent is selected from Cy3, Cy3.5, Cy5, Cy5.5, Sulfo Cy3, Sulfo Cy3.5, Sulfo Cy5, and Sulfo Cy5.5. In some embodiments, the imaging agent is selected from Cy5, Cy5.5, Sulfo Cy5, and Sulfo Cy5.5. In some embodiments, the imaging agent is Cy5 or Sulfo Cy5.

In some embodiments, the HER2-targeting compounds of the present disclosure are bound to Sulfo Cy5:

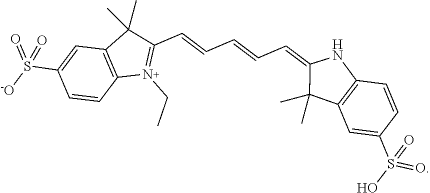

In some embodiments, M is an imaging agent attached to L¹, and L¹ is a linker attached to a side chain of an amino acid of the cyclic peptide (P). In further embodiments, M is an imaging agent, and L′ is a linker attached to a side chain NH₂ of an amino acid of the cyclic peptide. In still further embodiments, M is an imagining agent and L′ is a linker attached to a side chain NH₂ of A¹ or A¹⁰ of the cyclic peptide. In yet further embodiments, M is an imagining agent and L¹ is a bond or a linker attached to a lysine side chain or a β-alanine side chain of the cyclic peptide, wherein the lysine or β-alanine is at position A¹ or A¹⁰.

In some embodiments, L¹ is a linker of formula (L¹-II), and A¹, L¹, and M, together, have the structure:

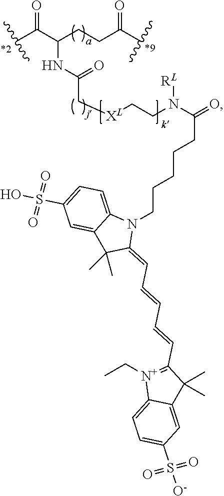

wherein:
X^{L} is selected from CH₂, N(R^{L}), and O;
R^{L} is selected from H and C₁₋₆-alkyl;
a is 1, 2, 3, or 4;
j′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
k′ is 0, 1, 2, 3, or 4;
*2 indicates the point of attachment to A²; and
*9 indicates the point of attachment to A⁹.

In some embodiments, L¹ is a linker of formula (L¹-II), and A¹⁰, L¹, and M, together, have the structure:

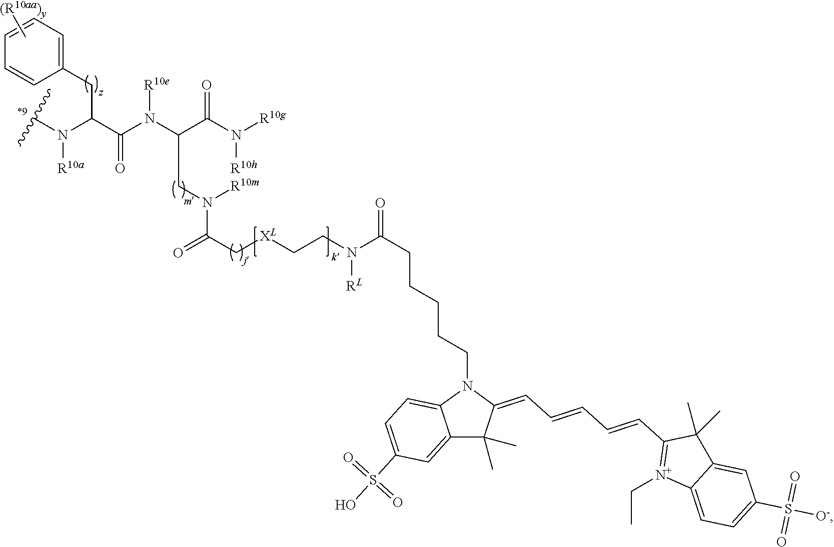

wherein:
X^{L} is selected from CH₂, N(R^{L}), and O;
R^{L} is selected from H and C₁₋₆-alkyl;
R^{10a} and R^{10e} are each independently selected from H and C₁₋₃-alkyl;
R^{10g} and R^{10h} are each independently selected from H and C₁₋₆-alkyl;
R^{10m} is selected from H and C₁₋₃-alkyl;
R^{10aa} is selected from C₁₋₆-alkyl, halo, —NH₂, —N₃, —OH, and —OC₁₋₆-alkyl;
a is 1, 2, 3, or 4;
j′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30;
k′ is 0, 1, 2, 3, or 4;
m′ is 1, 2, 3, 4, 5, or 6;
y is 0, 1, or 2;
z is 1, 2, 3, 4, 5, or 6; and
*9 indicates the point of attachment to A⁹.

In some embodiments, the HER2-targeting compounds of the present disclosure are bound to:

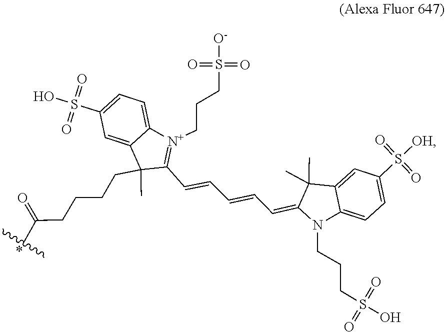 [SMILES: *C(=O)CCCCC1(C)C(/C=C/C=C/C=C2/N(CCCS(=O)(=O)O)c3ccc(S(=O)(=O)O)cc3C2(C)C)=[N+](CCCS(=O)(=O)[O-])c2ccc(S(=O)(=O)O)cc21]

wherein * represents the point of attachment to any NH₂ on P or L¹.

In certain embodiments of HER2-targeting compounds of the present disclosure, L¹ is a bond and M is an imaging agent attached to a side chain of A¹⁰, where M, L¹, and A¹⁰, together, have the structure:

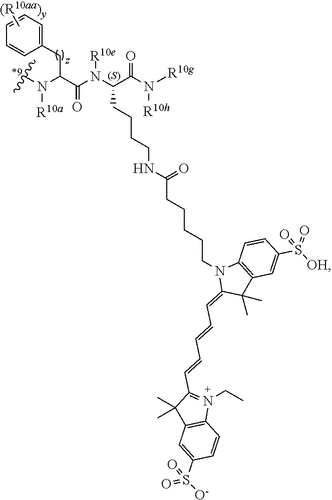

wherein *9 indicates the point of attachment to A⁹, and wherein the variables are defined elsewhere herein.

In certain embodiments of HER2-targeting compounds of the present disclosure, Mis an imaging agent and L¹ is a linker of formula (L¹-I) attached to a side chain A¹⁰, where M, L¹, and A¹⁰, together, have the structure:

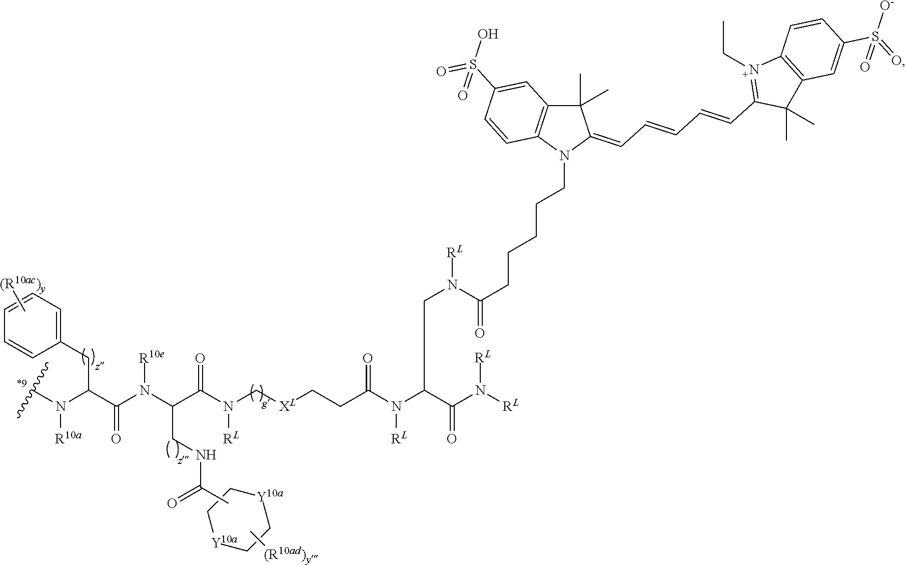

wherein *9 indicates the point of attachment to A⁹, and wherein the variables are defined elsewhere herein.

In certain embodiments of HER2-targeting compounds of the present disclosure, L¹ is a bond and M is an imaging agent attached to a side chain of A¹, where M, L¹, and A¹, together, have the structure:

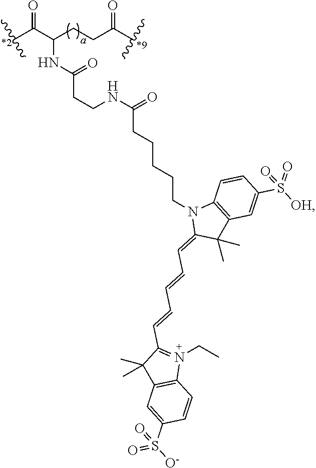

wherein *2 indicates the point of attachment to A², *9 indicates the point of attachment to A⁹, and wherein the variables are defined elsewhere herein.

In some embodiments, the compounds disclosed herein comprise both a radionuclide, which may be suitable for imaging (i.e., diagnostically active) or therapy, and a non-radioactive imaging agent.

In some embodiments, M is an imaging agent attached to L′, and L¹ is a linker attached to a side chain of an amino acid of the cyclic peptide (P). In further embodiments, M is an imaging agent, and L¹ is a bond or a linker attached to a side chain NH₂ of an amino acid of cyclic peptide P. In yet further embodiments, M is an imagining agent and L¹ is a bond or a linker attached to a lysine side chain or a β-alanine side chain of the cyclic peptide, wherein the lysine or β-alanine is at position A¹ or A¹⁰.

In some embodiments, M, L¹, and A¹⁰, together, have one of the following structures:

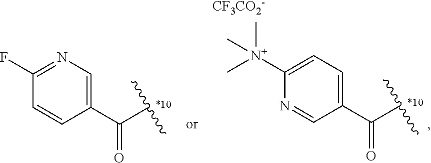 [SMILES: *C(=O)c1ccc(F)nc1 ; "CF3CO2-" ; *C(=O)c1ccc([N+](C)(C)C)nc1]

wherein *10 indicates the point of attachment to A¹⁰.

In some embodiments, M, L¹, and A¹⁰, together, have one of the following structures:

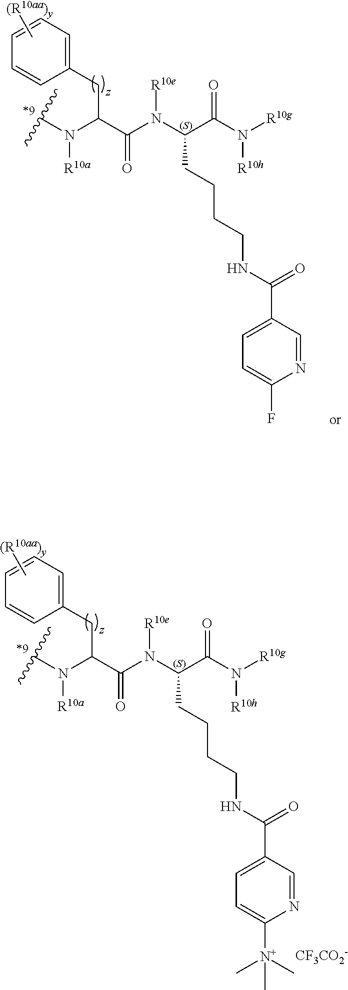

wherein *9 indicates the point of attachment to A⁹, and wherein the variables are defined elsewhere herein.

### Chelators

In one or more embodiments of the HER2-targeting compounds described herein, M is a chelator. As used herein, the terms “chelator,” “chelating ligand,” and “chelating agent” are interchangeable and typically refer to chemical moieties, agents, compounds, or molecules able to form a complex containing one or more coordinate bonds with a metal ion. The chelators described herein may form a complex with any metal element or metal ion known in the art (e.g. any element of ion of alkali metals, alkaline earth metals, transition metals, inner transition metals (lanthanides, actinides), metalloids, or poor metals, said metals being present alone or in connection with another element, e.g. an ion of a metal in connection with a halogen, e.g. a metal-halogenide) to coordinate to a chelating agent. In some embodiments, the chelator is a nuclide chelator. In some embodiments, the chelator is a radionuclide chelator.

Chelators that form a complex with a metal ion, such as a radionuclide, may be referred to herein as radiolabeled chelators. In some embodiments, a chelator complexed or coordinated to a metal ion is depicted with single bonds between heteroatoms of the chelator and the metal ion or is depicted with dashed bonds between the heteroatoms of the chelator and the metal ion.

In some embodiments, the chelator forms a complex with a metal ion and with one or more additional molecules, e.g., a water molecule.

When the chelator is chelated or complexed to a radionuclide, it can be said that the chelator is radiolabeled. In some embodiments of the compounds described herein, M is a chelator, and the resulting conjugate of the cyclic peptide with a chelator and optionally a linker can be radiolabeled with a radionuclide.

In some embodiments, chelating agents suitable for coordinating to metal ions may have a cyclic or acyclic structure. In some embodiments, chelating agents suitable for coordinating to metal ions may have a cyclic or linear structure.

In some embodiments, the chelator is a cyclic chelator. As used herein, the term “cyclic chelator” means any chelator having a heterocyclic ring in which two or more of the ring heteroatoms form a coordinate covalent bond to a metal ion, such as, for example, a radionuclide. In an embodiment, the heterocyclic ring has at least two heteroatoms selected from the group consisting of nitrogen, oxygen, and sulfur. In another embodiment, the heterocyclic ring is a macrocycle having between 7 and 30 ring atoms. In yet another embodiment, the heterocyclic ring is a macrocycle having between 7 and 20 ring atoms. In still another embodiment, the heterocyclic ring is a macrocycle having between 7 and 15 ring atoms. In an embodiment, the cyclic chelator is DOTA.

In some embodiments, the chelator is an acyclic chelator. As used herein, the term “acyclic chelator” means any chelator having an open chain that contains heteroatoms in which two or more of the heteroatoms form a coordinate covalent bond to a metal ion, such as, for example, a radionuclide. An acyclic chelator may comprise cycloalkyl, heterocyclic, aryl, and heteroaryl rings, or a combination thereof. However, when the acyclic chelator comprises heterocyclic and/or heteroaryl rings, no more than one heteroatom of each ring participates in the coordinate covalent bond with the metal ion. In an embodiment, the acyclic chelator is a linear chelator, such as, for example, H2dedpa, BFO, DFO, and mertiatide. In another embodiment, the acyclic chelator is a branched chelator, such as, for example, EDTA, DTPA, Pip-DTPA, ECC, ECD, and citric acid.

Suitable cyclic chelators may have a heterocyclic core, such as, for example, a crown ether or an aza-crown ether. In an embodiment, the heterocyclic core is an aza-crown ether. In another embodiment, the heterocyclic core comprises nitrogen and oxygen heteroatoms. In yet another embodiment, the heterocyclic core is a crown ether.

Chelators in accordance with the present disclosure may have a heterocyclic core comprising from 2 to 10 heteroatoms. For example, the chelator may have a heterocyclic core comprising 2, 3, 4, 5, 6, 7, 8, 9, or 10 heteroatoms. In some embodiments, heterocyclic core comprises from 2 to 8 heteroatoms, from 3 to 8 heteroatoms, from 2 to 6 heteroatoms or from 3 to 6 heteroatoms. In an embodiment, the heterocyclic core comprises 3 heteroatoms. In another embodiment, the heterocyclic core comprises 4 heteroatoms. In another embodiment, the heterocyclic core comprises 5 heteroatoms. In yet another embodiment, the heterocyclic core comprises 6 heteroatoms.

In some embodiments, one or more heteroatoms of the core structure is functionalized. For example, heteroatoms of the cyclic core may be functionalized with one or more of H, OH, C(O)OR^{x}, CR^{x}₂C(O)R^{x}, (CR^{x})ₙC(O)OR^{x}, (CR^{x})ₙC(O)NH₂, (CR^{x})ₙC(O)NHR^{x}, (CR^{x})ₙC(O)NR^{x}₂, CR^{x}₂P(O)(OR^{x})₂, (CR)ₙP(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SCN, C(O)OR^{x}, C(O)NRx, P(O)(OR^{x})₂, N₃, NR^{x}₂, SR^{x}, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SCN, NH₂, CH₂C(O)OH, C(O)OH, halo, alkyl, haloalkyl, alkylaryl, heteroaryl, cycloalkyl, heterocycloalkyl, and P(O)(OH)₂ and n is 0, 1, 2, 3, 4, or 5.

In some embodiments, the heterocyclic core of the chelator comprises heteroatoms separated by alkylene groups, such as, for example, methylene, ethylene, or propylene groups. In some embodiments, the heteroatoms of the heterocyclic core are separated by ethylene groups. In some embodiments, the heteroatoms of the heterocyclic core are separated by methylene groups. The alkylene groups separating the heteroatoms of the core structure may be substituted or unsubstituted. In some embodiments, one or more alkylene groups of the heterocyclic core are substituted with one or more H, OH, C(O)OR^{x},CR^{x}₂C(O)OR^{x}, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SCN, C(O)OR^{x}, C(O)NR^{x}, P(O)(OR)₂, N₃, NR₂, SR^{x}, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SCN, NH₂, CH₂C(O)OH, C(O)OH, halo, alkyl, alkoxy, haloalkyl, alkylaryl, heteroaryl, cycloalkyl, heterocycloalkyl, P(O)(OH)₂, and SH.

In an embodiment, the cyclic chelator comprises a 1,4,7,10-tetraazacyclododecane core or a residue thereof, such as, for example, the structures shown in Table 3 or residues thereof, below.

TABLE 3
Exemplary Cyclic Chelators with a 1,4,7,10-tetraazacyclododecane core

| | |
|---|---|
| DOTA | 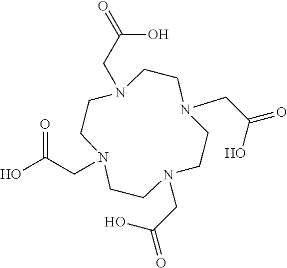 [SMILES: O=C(O)CN1CCN(CC(=O)O)CCN(CC(=O)O)CCN(CC(=O)O)CC1] |
| DOTMP | 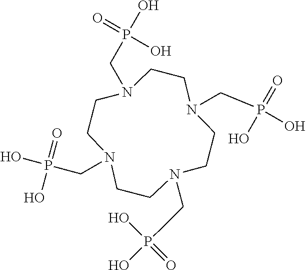 [SMILES: O=P(O)(O)CN1CCN(CP(=O)(O)O)CCN(CP(=O)(O)O)CCN(CP(=O)(O)O)CC1] |
| DO3A | 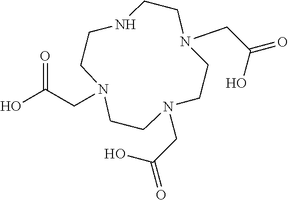 [SMILES: O=C(O)CN1CCNCCN(CC(=O)O)CCN(CC(=O)O)CC1] |
| DOTA-AA | 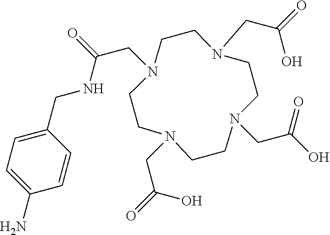 [SMILES: Nc1ccc(CNC(=O)CN2CCN(CC(=O)O)CCN(CC(=O)O)CCN(CC(=O)O)CC2)cc1] |
| DOTASA | 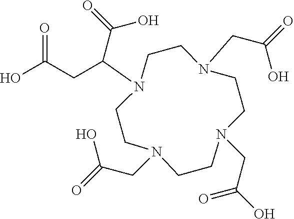 [SMILES: O=C(O)CC(C(=O)O)N1CCN(CC(=O)O)CCN(CC(=O)O)CCN(CC(=O)O)CC1] |
| DOTAZA | 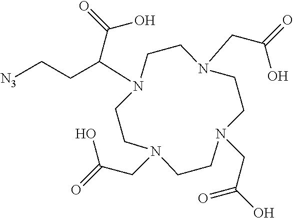 [SMILES: [N-]=[N+]=NCCC(C(=O)O)N1CCN(CC(=O)O)CCN(CC(=O)O)CCN(CC(=O)O)CC1] |
| DOTAGA | 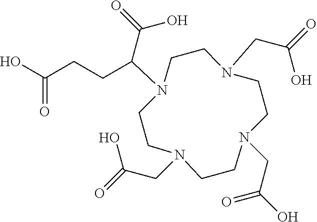 [SMILES: O=C(O)CCC(C(=O)O)N1CCN(CC(=O)O)CCN(CC(=O)O)CCN(CC(=O)O)CC1] |
| | 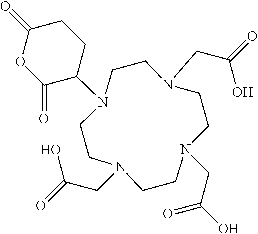 [SMILES: O=C(O)CN1CCN(CC(=O)O)CCN(C2CCC(=O)OC2=O)CCN(CC(=O)O)CC1] |
| Me- DO2PA | 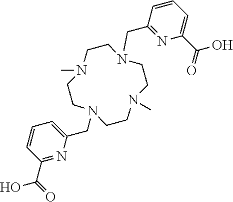 [SMILES: CN1CCN(Cc2cccc(C(=O)O)n2)CCN(C)CCN(Cc2cccc(C(=O)O)n2)CC1] |
| DOTPA | 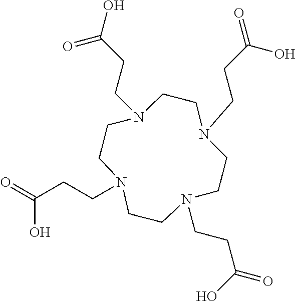 [SMILES: O=C(O)CCN1CCN(CCC(=O)O)CCN(CCC(=O)O)CCN(CCC(=O)O)CC1] |
| DEPA | 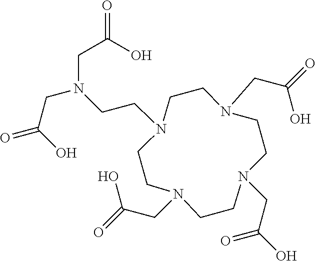 [SMILES: O=C(O)CN1CCN(CCN(CC(=O)O)CC(=O)O)CCN(CC(=O)O)CCN(CC(=O)O)CC1] |
| p-SCN-Bn- DOTA | 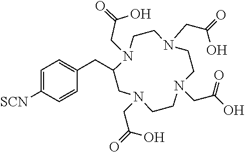 |
| C-DEPA | 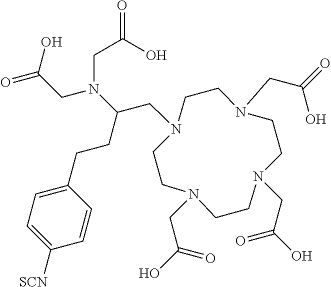 |
| MeO- DOTA- NCS | 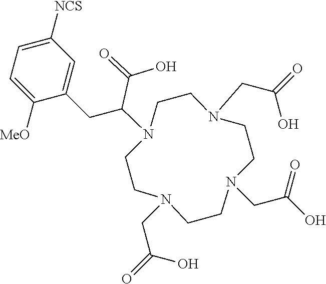 |
| 3p-C- DEPA | 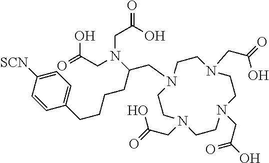 |
| DO3A1P | 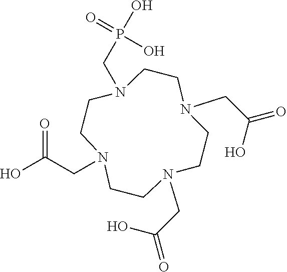 [SMILES: O=C(O)CN1CCN(CC(=O)O)CCN(CP(=O)(O)O)CCN(CC(=O)O)CC1] |
| 3p-C- DE4TA | 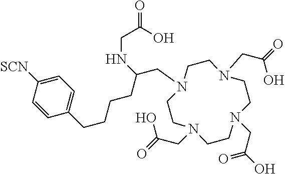 |
| DE4TA | 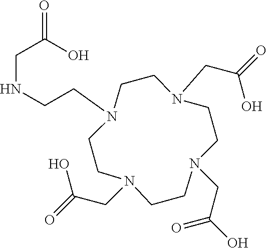 [SMILES: O=C(O)CNCCN1CCN(CC(=O)O)CCN(CC(=O)O)CCN(CC(=O)O)CC1] |
| DODASA | 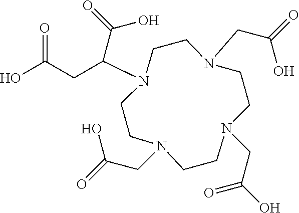 [SMILES: O=C(O)CC(C(=O)O)N1CCN(CC(=O)O)CCN(CC(=O)O)CCN(CC(=O)O)CC1] |
| PCS | 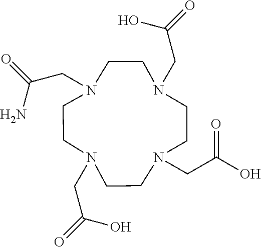 [SMILES: NC(=O)CN1CCN(CC(=O)O)CCN(CC(=O)O)CCN(CC(=O)O)CC1] |
| DOTAM (also referred to as TCMC) | 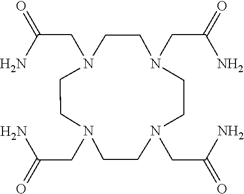 [SMILES: NC(=O)CN1CCN(CC(N)=O)CCN(CC(N)=O)CCN(CC(N)=O)CC1] |

In another embodiment, the cyclic chelator comprises a 1,4,7-triazacyclononane core, such as, for example, the structures shown in Table 4, below.

TABLE 4
Exemplary Cyclic Chelators with a 1,4,7-triazacyclononane core

| | |
|---|---|
| NOTA | 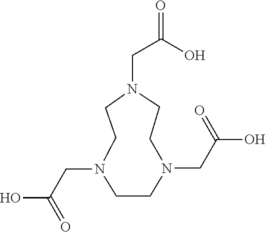 [SMILES: O=C(O)CN1CCN(CC(=O)O)CCN(CC(=O)O)CC1] |
| TRAP | 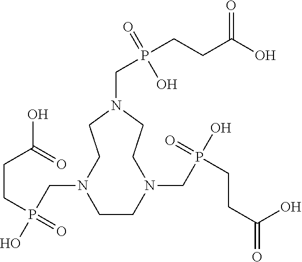 [SMILES: O=C(O)CCP(=O)(O)CN1CCN(CP(=O)(O)CCC(=O)O)CCN(CP(=O)(O)CCC(=O)O)CC1] |
| NS3 | 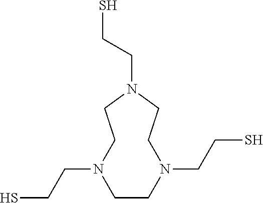 [SMILES: SCCN1CCN(CCS)CCN(CCS)CC1] |
| NETA | 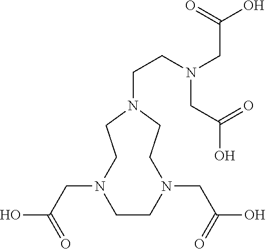 [SMILES: O=C(O)CN1CCN(CCN(CC(=O)O)CC(=O)O)CCN(CC(=O)O)CC1] |
| NODAGA | 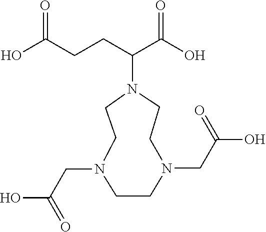 [SMILES: O=C(O)CCC(C(=O)O)N1CCN(CC(=O)O)CCN(CC(=O)O)CC1] |
| C-NETA | 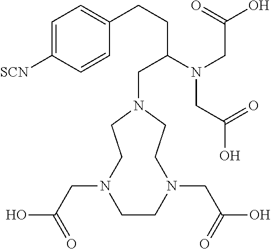 |
| p-SCN- Bn-NOTA | 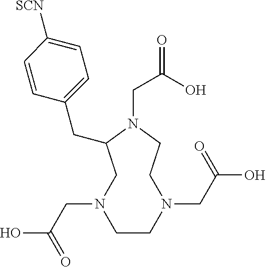 |
| 3p-C-NETA | 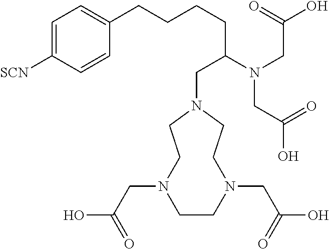 |
| NO2A | 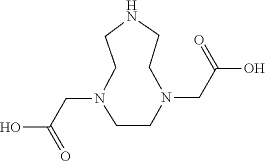 [SMILES: O=C(O)CN1CCNCCN(CC(=O)O)CC1] |
| NESTA | 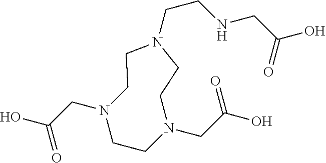 [SMILES: O=C(O)CNCCN1CCN(CC(=O)O)CCN(CC(=O)O)CC1] |
| NO2AP | 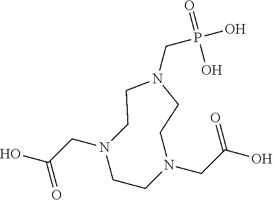 [SMILES: O=C(O)CN1CCN(CC(=O)O)CCN(CP(=O)(O)O)CC1] |
| C-NESTA | 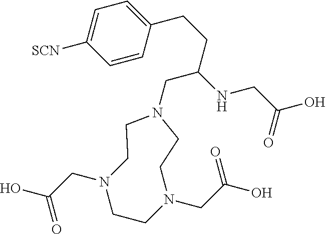 |
| NOTP | 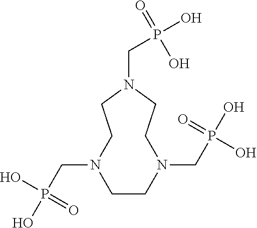 [SMILES: O=P(O)(O)CN1CCN(CP(=O)(O)O)CCN(CP(=O)(O)O)CC1] |
| 3p-C-NESTA | 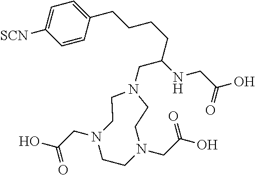 |
| NOPO | 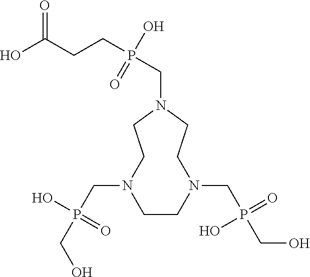 [SMILES: O=C(O)CCP(=O)(O)CN1CCN(CP(=O)(O)CO)CCN(CP(=O)(O)CO)CC1] |
| NODIA-Me | 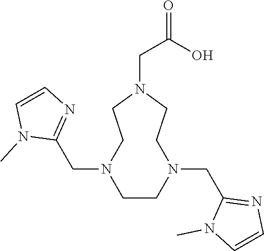 [SMILES: Cn1ccnc1CN1CCN(CC(=O)O)CCN(Cc2nccn2C)CC1] |

In some embodiments, the cyclic chelator comprises a bridged heterocyclic core. In some embodiments, the cyclic chelator comprises a fused heterocyclic core. For example, in one or more embodiments, the heterocyclic core (of the cyclic chelator) may be fused to a heteroaryl or heterocycloalkyl ring, such that the two cycles share a heteroatom. Suitable heteroaryl and heterocycloalkyl rings that may be fused to the heterocyclic core (of the cyclic chelator) include, but are not limited to pyridine, pyrimidine, furan, tetrahydropyran, pyran, dioxane, and oxazole, imidazole, and pyrrole.

Chelators comprising a fused or bridged core structure, as well as various other suitable heterocyclic cores, include, but are not limited to, the structures shown in Table 5, below.

TABLE 5
Additional Exemplary Cyclic Chelators

| | |
|---|---|
| CB-DO2A | 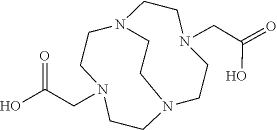 [SMILES: O=C(O)CN1CCN2CCN(CC1)CCN(CC(=O)O)CC2] |
| L3 | 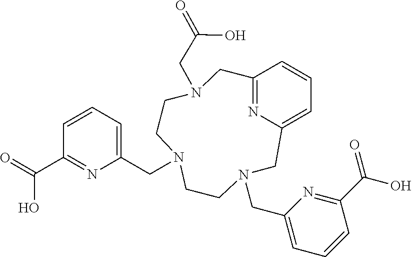 [SMILES: O=C(O)CN1CCN(Cc2cccc(C(=O)O)n2)CCN(Cc2cccc(C(=O)O)n2)Cc2cccc(n2)C1] |
| TRITA | 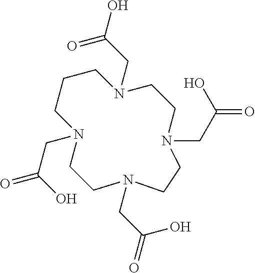 [SMILES: O=C(O)CN1CCCN(CC(=O)O)CCN(CC(=O)O)CCN(CC(=O)O)CC1] |
| H2macropa | 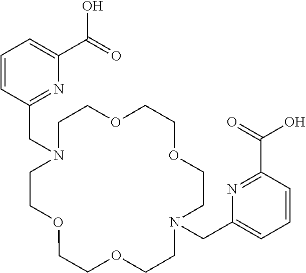 [SMILES: O=C(O)c1cccc(CN2CCOCCOCCN(Cc3cccc(C(=O)O)n3)CCOCCOCC2)n1] |
| TETA | 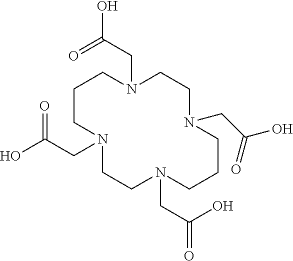 [SMILES: O=C(O)CN1CCCN(CC(=O)O)CCN(CC(=O)O)CCCN(CC(=O)O)CC1] |
| p-SCN-Bn-macropa | 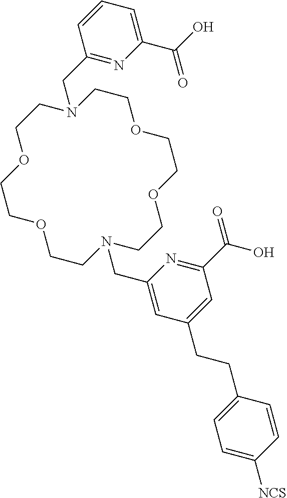 |
| TE2A | 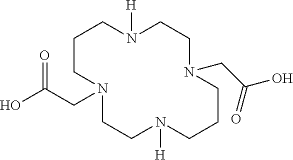 [SMILES: O=C(O)CN1CCCNCCN(CC(=O)O)CCCNCC1] |
| PEPA | 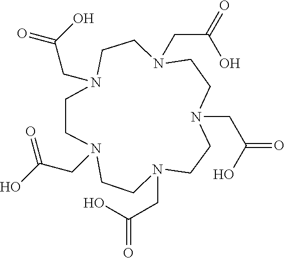 [SMILES: O=C(O)CN1CCN(CC(=O)O)CCN(CC(=O)O)CCN(CC(=O)O)CCN(CC(=O)O)CC1] |
| TETPA | 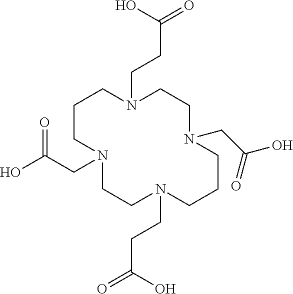 [SMILES: O=C(O)CCN1CCCN(CC(=O)O)CCN(CCC(=O)O)CCCN(CC(=O)O)CC1] |
| p-SCN-Bn-PEPA | 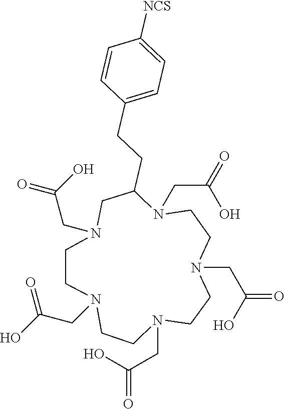 |
| PCTA | 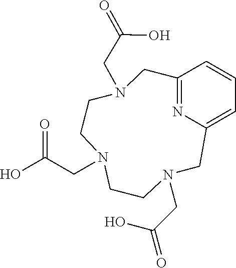 [SMILES: O=C(O)CN1CCN(CC(=O)O)Cc2cccc(n2)CN(CC(=O)O)CC1] |
| HEHA | 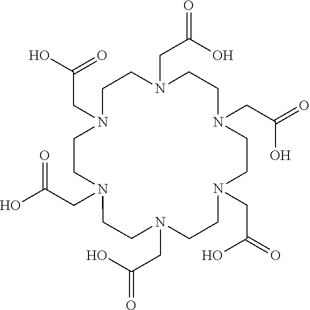 [SMILES: O=C(O)CN1CCN(CC(=O)O)CCN(CC(=O)O)CCN(CC(=O)O)CCN(CC(=O)O)CCN(CC(=O)O)CC1] |
| p-SCN-Bn-PCTA | 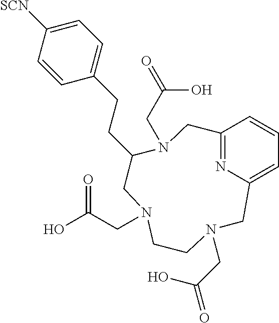 |
| p-SCN-Bn-HEHA | 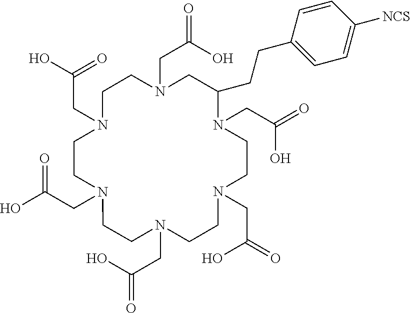 |
| L1 | 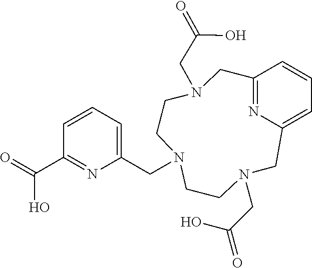 [SMILES: O=C(O)CN1CCN(Cc2cccc(C(=O)O)n2)CCN(CC(=O)O)Cc2cccc(n2)C1] |
| FSC | 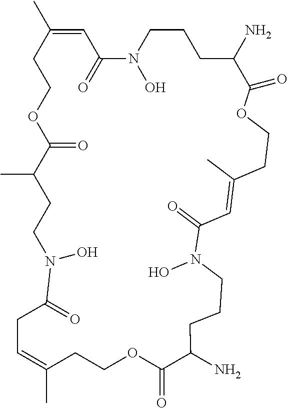 [SMILES: C/C1=C/C(=O)N(O)CCCC(N)C(=O)OCC/C(C)=C/C(=O)N(O)CCCC(N)C(=O)OCC/C(C)=C\CC(=O)N(O)CCC(C)C(=O)OCC1] |
| L2 | 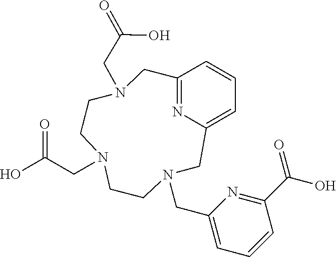 [SMILES: O=C(O)CN1CCN(CC(=O)O)Cc2cccc(n2)CN(Cc2cccc(C(=O)O)n2)CC1] |
| AAZTA | 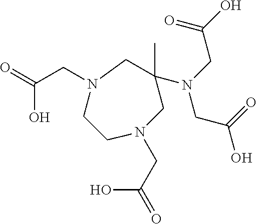 [SMILES: CC1(N(CC(=O)O)CC(=O)O)CN(CC(=O)O)CCN(CC(=O)O)C1] |
| CNAAZTA | 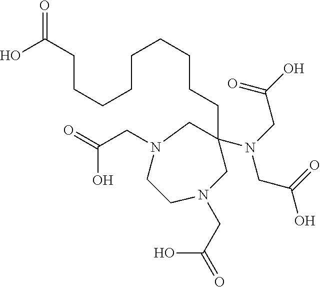 [SMILES: O=C(O)CCCCCCCCCC1(N(CC(=O)O)CC(=O)O)CN(CC(=O)O)CCN(CC(=O)O)C1] |
| DATA^{m} | 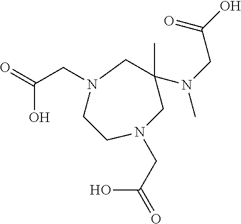 [SMILES: CN(CC(=O)O)C1(C)CN(CC(=O)O)CCN(CC(=O)O)C1] |
| AAZTA5 | 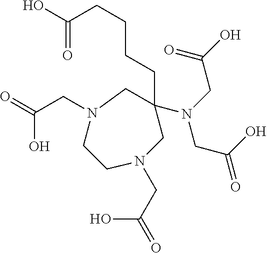 [SMILES: O=C(O)CCCCC1(N(CC(=O)O)CC(=O)O)CN(CC(=O)O)CCN(CC(=O)O)C1] |
| H₂bispa² | 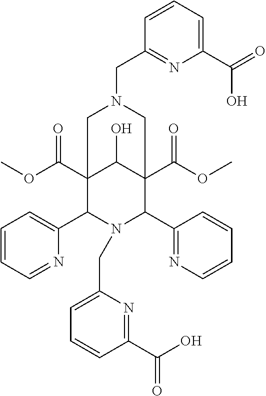 [SMILES: COC(=O)C12CN(Cc3cccc(C(=O)O)n3)CC(C(=O)OC)(C(c3ccccn3)N(Cc3cccc(C(=O)O)n3)C1c1ccccn1)C2O] |
| bispidine | 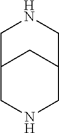 |
| bispidine-L₁ | 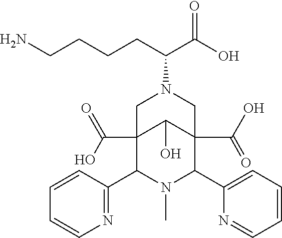 [SMILES: CN1C(c2ccccn2)C2(C(=O)O)CN([C@H](CCCCN)C(=O)O)CC(C(=O)O)(C1c1ccccn1)C2O] |
| CB-TE2A | 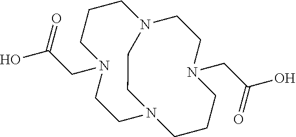 [SMILES: O=C(O)CN1CCCN2CCN(CCCN(CC(=O)O)CC2)CC1] |
| Sarcophagine | 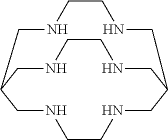 [SMILES: C1CNCC2CNCCNCC(CN1)CNCCNC2] |
| macropa-XL | 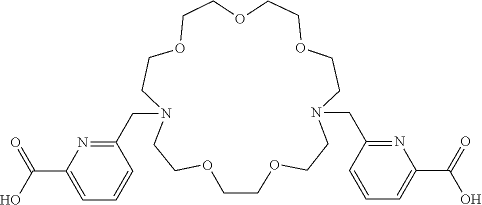 [SMILES: O=C(O)c1cccc(CN2CCOCCOCCOCCN(Cc3cccc(C(=O)O)n3)CCOCCOCC2)n1] |
| DB30C10 | 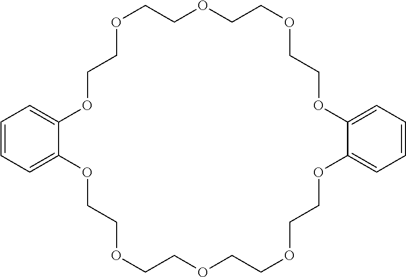 [SMILES: c1ccc2c(c1)OCCOCCOCCOCCOc1ccccc1OCCOCCOCCOCCO2] |

In some embodiments, the chelator is an acyclic or linear chelator. Suitable linear chelating ligands may comprise a series of functionalized amines separated by alkylene, arylene, cycloalkyl, heteroaryl, heterocycloalkyl groups, or a combination thereof. In some embodiments, the acyclic chelator comprises 2, 3, or 4 functionalized amines. For example, in some embodiments the linear chelator comprises 2, 3, or 4 amines functionalized with one or more of H, OH, C(O)OR^{x},CR^{x}₂C(O)OR^{x}, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SCN, C(O)OR^{x}, C(O)NRx, P(O)(OR^{x})₂, N₃, NR^{x}₂, SR, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SCN, NH₂, CH₂COOH, C(O)OH, halo, alkyl, haloalkyl, alkylaryl, heteroaryl, cycloalkyl, heterocycloalkyl, and P(O)(OH)₂.

In some embodiments, the acyclic chelator comprises a series of functionalized amines separated by alkylene groups, such as, for example, methylene, ethylene, and propylene groups. The alkylene groups separating the functionalized amines may be substituted or unsubstituted. In some embodiments, one or more of the alkylene groups separating the functionalized amines are substituted with one or more of H, OH, C(O)OR^{x},CR^{x}₂C(O)OR, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SO₃, SCN, C(O)OR^{x}, C(O)NRx, P(O)(OR^{x})₂, N₃, NR^{x}₂, SR^{x}, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SO₃⁻, SCN, NH₂, CH₂C(O)OH, C(O)OH, halo, alkyl, haloalkyl, alkylaryl, heteroaryl, cycloalkyl, heterocycloalkyl, and P(O)(OH)₂.

In an embodiment, the linear chelator comprises a series of functionalized amines separated by methylene groups. In another embodiment, the acyclic or linear chelator comprises a series of functionalized amines separated by ethylene groups, such as, for example, the structures provided in Table 6, below.

TABLE 6
Exemplary Acyclic Chelators

| | |
|---|---|
| EDTA | 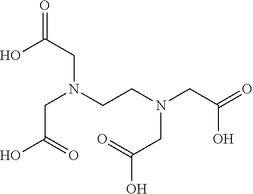 [SMILES: O=C(O)CN(CCN(CC(=O)O)CC(=O)O)CC(=O)O] |
| 1B4M-DTPA | 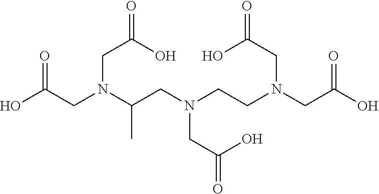 [SMILES: CC(CN(CCN(CC(=O)O)CC(=O)O)CC(=O)O)N(CC(=O)O)CC(=O)O] |
| EDTMP | 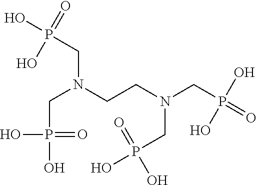 [SMILES: O=P(O)(O)CN(CCN(CP(=O)(O)O)CP(=O)(O)O)CP(=O)(O)O] |
| p-SCN-Bn-DTPA | 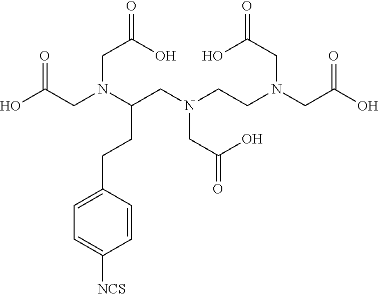 |
| HBED | 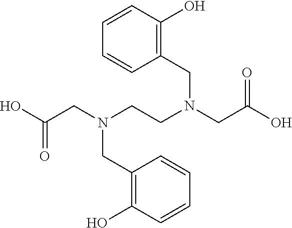 [SMILES: O=C(O)CN(CCN(CC(=O)O)Cc1ccccc1O)Cc1ccccc1O] |
| DTPA-amide | 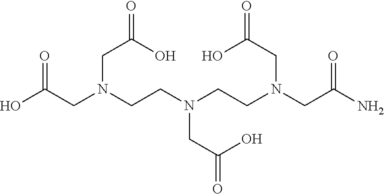 [SMILES: NC(=O)CN(CCN(CCN(CC(=O)O)CC(=O)O)CC(=O)O)CC(=O)O] |
| SHBED | 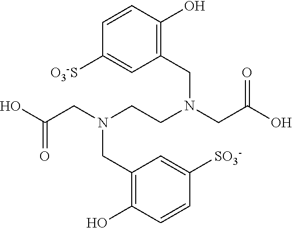 |
| H₄neunpa | 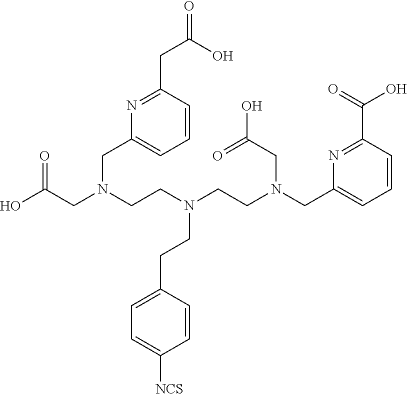 |
| H₆Sbbpen | 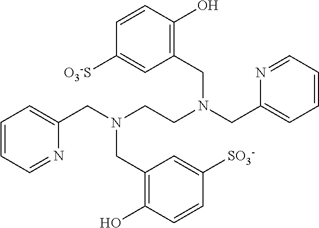 |
| HBED-CC | 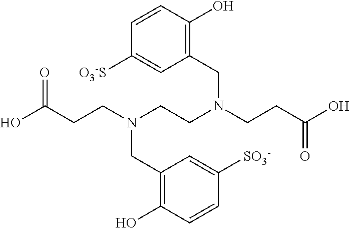 |
| TTHA | 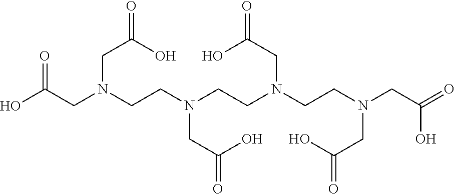 [SMILES: O=C(O)CN(CCN(CCN(CC(=O)O)CC(=O)O)CC(=O)O)CCN(CC(=O)O)CC(=O)O] |
| DTPA | 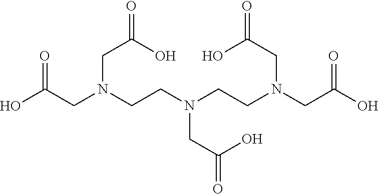 [SMILES: O=C(O)CN(CCN(CC(=O)O)CC(=O)O)CCN(CC(=O)O)CC(=O)O] |
| TTHMP | 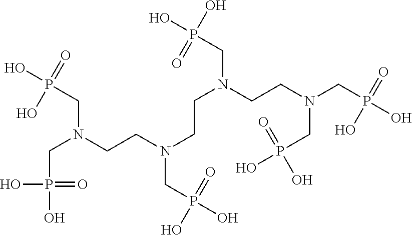 [SMILES: O=P(O)(O)CN(CCN(CCN(CP(=O)(O)O)CP(=O)(O)O)CP(=O)(O)O)CCN(CP(=O)(O)O)CP(=O)(O)O] |

The acyclic chelator may comprise a variety of heteroatoms, such as, for example, nitrogen, oxygen, and sulfur heteroatoms. In some embodiments, the acyclic chelator comprises nitrogen heteroatoms. In some embodiments, the acyclic chelator comprises nitrogen and oxygen heteroatoms. In some embodiments, the acyclic chelator comprises nitrogen and sulfur heteroatoms.

One or more of the heteroatoms of the acyclic or linear chelator may be functionalized. For example, in some embodiments the acyclic or linear chelator comprises one or more heteroatoms functionalized with phosphoric acid, carboxylic acid, substituted or unsubstituted aryl, substituted or unsubstituted phenyl, substituted or unsubstituted heteroaryl, amide, and substituted or unsubstituted alkyl groups. Substituted aryl, benzyl, heteroaryl, and alkyl functionalized amines may be substituted with one or more hydroxyl, carboxyl, alkyl, carbonyl, cyano, phosphoryl, alkoxy, and aryl groups.

One or more heteroatoms of an acyclic chelator may be from a heteroarene, such as, for example, pyridine, pyrrole, thiophene, and furan. One or more heteroatoms of an acyclic chelator may be from a carbonyl functionality, such as, for example, an amide, an ester, an acid, and a thioester. In some embodiments, two or more, but not all, of the heteroatoms in the acyclic core may be connected via a heterocyclyl structure.

In some embodiments, the linear chelator comprises a series of heteroatoms separated by a combination of alkylene and arylene groups. In some embodiments, the linear chelator comprises a series of functionalized amines separated by a combination of alkylene and cycloalkyl groups. Alkylene, arylene, and cycloalkyl groups separating the heteroatoms of a linear chelator may be substituted or unsubstituted. Further suitable acyclic chelating ligands include, but are not limited to, the structures shown in Table 7, below.

TABLE 7
Additional Exemplary Acyclic Chelators

| | |
|---|---|
| Pip-DTPA | 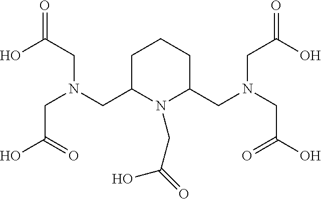 [SMILES: O=C(O)CN(CC(=O)O)CC1CCCC(CN(CC(=O)O)CC(=O)O)N1CC(=O)O] |
| CHX-DTPA | 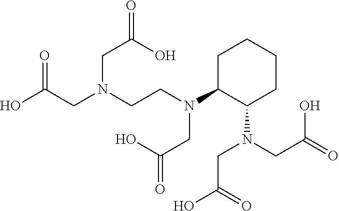 [SMILES: O=C(O)CN(CCN(CC(=O)O)[C@H]1CCCC[C@@H]1N(CC(=O)O)CC(=O)O)CC(=O)O] |
| p-SCN-Bn-CHX-A″-DTPA | 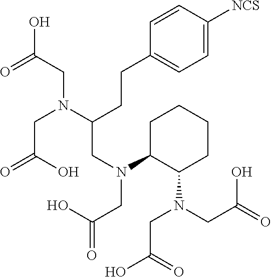 |
| 3,2-HOPO | 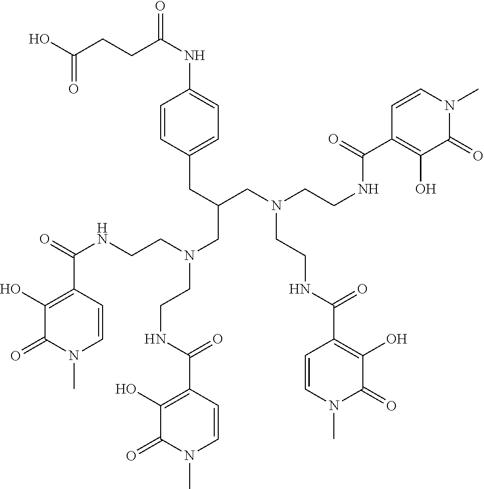 [SMILES: Cn1ccc(C(=O)NCCN(CCNC(=O)c2ccn(C)c(=O)c2O)CC(Cc2ccc(NC(=O)CCC(=O)O)cc2)CN(CCNC(=O)c2ccn(C)c(=O)c2O)CCNC(=O)c2ccn(C)c(=O)c2O)c(O)c1=O] |
| BATPA | 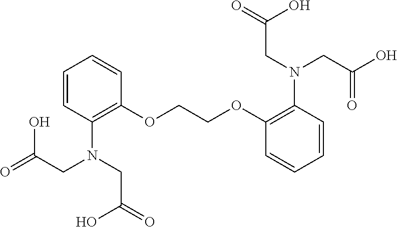 [SMILES: O=C(O)CN(CC(=O)O)c1ccccc1OCCOc1ccccc1N(CC(=O)O)CC(=O)O] |
| THP | 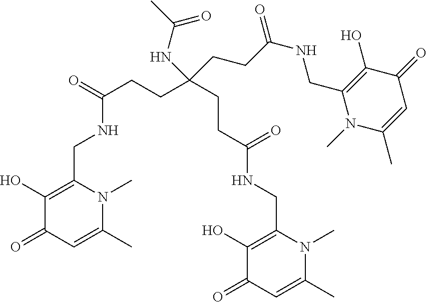 [SMILES: CC(=O)NC(CCC(=O)NCc1c(O)c(=O)cc(C)n1C)(CCC(=O)NCc1c(O)c(=O)cc(C)n1C)CCC(=O)NCc1c(O)c(=O)cc(C)n1C] |
| EGTA | 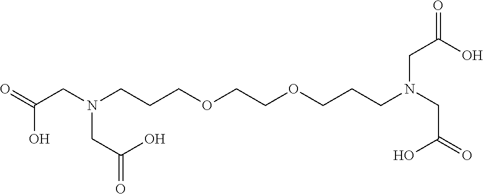 [SMILES: O=C(O)CN(CCCOCCOCCCN(CC(=O)O)CC(=O)O)CC(=O)O] |
| THP-NH2 | 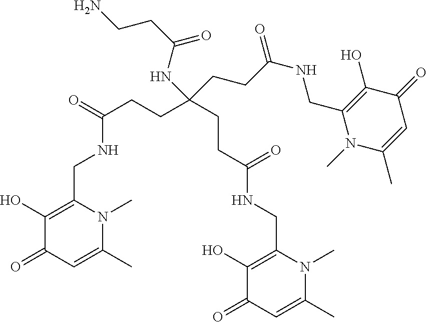 [SMILES: Cc1cc(=O)c(O)c(CNC(=O)CCC(CCC(=O)NCc2c(O)c(=O)cc(C)n2C)(CCC(=O)NCc2c(O)c(=O)cc(C)n2C)NC(=O)CCN)n1C] |
| B6SS | 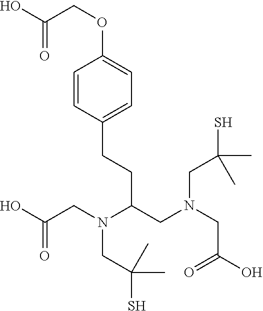 [SMILES: CC(C)(S)CN(CC(=O)O)CC(CCc1ccc(OCC(=O)O)cc1)N(CC(=O)O)CC(C)(C)S] |
| THP-NCS | 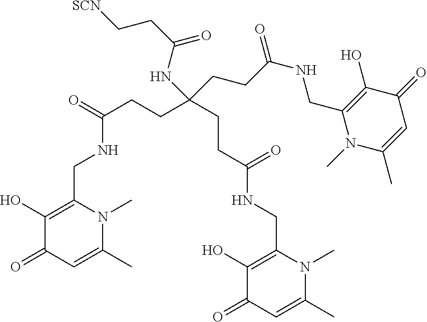 |
| H₂dedpa | 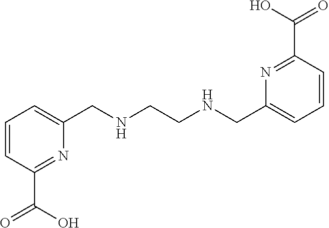 [SMILES: O=C(O)c1cccc(CNCCNCc2cccc(C(=O)O)n2)n1] |
| YM103 | 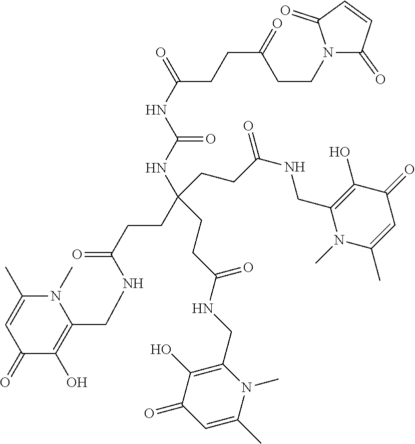 [SMILES: Cc1cc(=O)c(O)c(CNC(=O)CCC(CCC(=O)NCc2c(O)c(=O)cc(C)n2C)(CCC(=O)NCc2c(O)c(=O)cc(C)n2C)NC(=O)NC(=O)CCC(=O)CCN2C(=O)C=CC2=O)n1C] |
| p-SCN-Bn-dedpa | 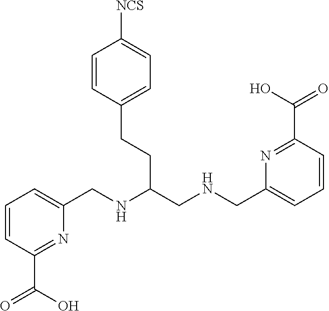 |
| ECC | 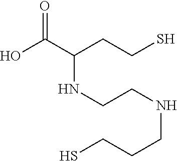 [SMILES: O=C(O)C(CCS)NCCNCCCS] |
| H₄octapa | 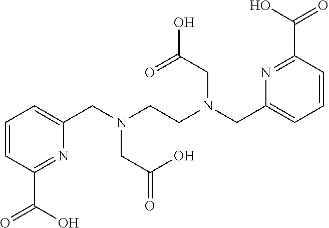 [SMILES: O=C(O)CN(CCN(CC(=O)O)Cc1cccc(C(=O)O)n1)Cc1cccc(C(=O)O)n1] |
| ECD | 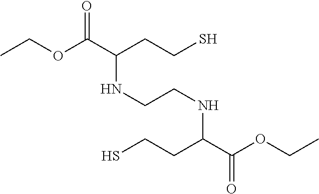 [SMILES: CCOC(=O)C(CCS)NCCNC(CCS)C(=O)OCC] |
| p-SCN-Bn-octapa | 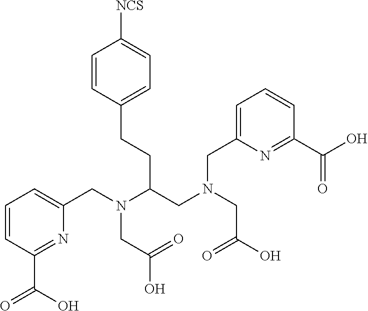 |
| Citric acid | 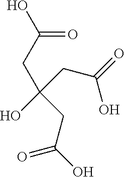 [SMILES: O=C(O)CC(O)(CC(=O)O)CC(=O)O] |
| BFO | 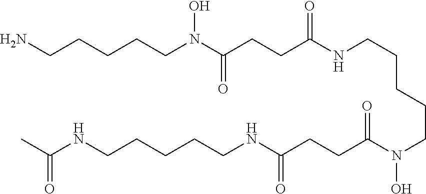 [SMILES: CC(=O)NCCCCCNC(=O)CCC(=O)N(O)CCCCCNC(=O)CCC(=O)N(O)CCCCCN] |
| 6SS | 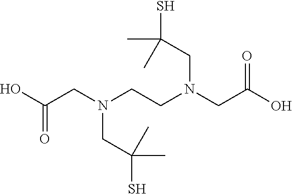 [SMILES: CC(C)(S)CN(CCN(CC(=O)O)CC(C)(C)S)CC(=O)O] |
| RESCA | 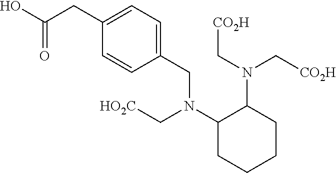 [SMILES: O=C(O)Cc1ccc(CN(CC(=O)O)C2CCCCC2N(CC(=O)O)CC(=O)O)cc1] |
| DFO | 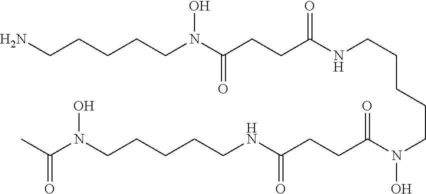 [SMILES: CC(=O)N(O)CCCCCNC(=O)CCC(=O)N(O)CCCCCNC(=O)CCC(=O)N(O)CCCCCN] |
| Pypa | 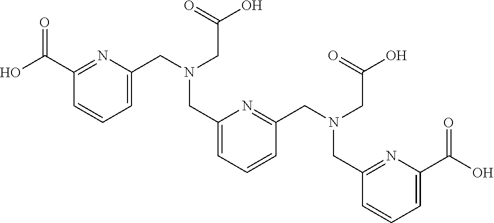 [SMILES: O=C(O)CN(Cc1cccc(CN(CC(=O)O)Cc2cccc(C(=O)O)n2)n1)Cc1cccc(C(=O)O)n1] |
| Py4pa | 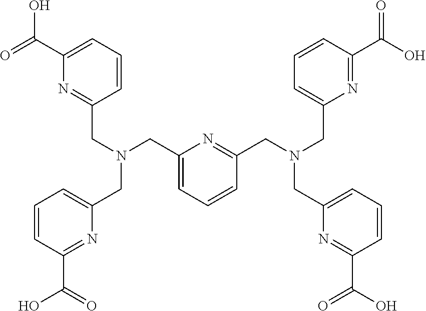 [SMILES: O=C(O)c1cccc(CN(Cc2cccc(CN(Cc3cccc(C(=O)O)n3)Cc3cccc(C(=O)O)n3)n2)Cc2cccc(C(=O)O)n2)n1] |
| H₂CHXhox | 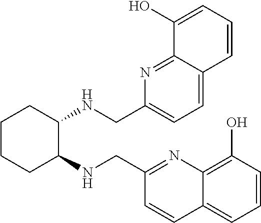 [SMILES: Oc1cccc2ccc(CN[C@H]3CCCC[C@@H]3NCc3ccc4cccc(O)c4n3)nc12] |
| CHXoctapa | 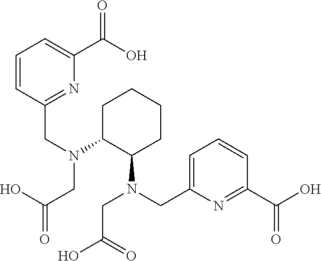 [SMILES: O=C(O)CN(Cc1cccc(C(=O)O)n1)[C@@H]1CCCC[C@H]1N(CC(=O)O)Cc1cccc(C(=O)O)n1] |
| H₂hox | 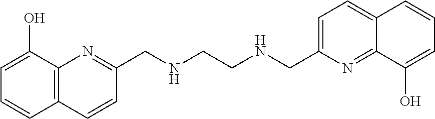 [SMILES: Oc1cccc2ccc(CNCCNCc3ccc4cccc(O)c4n3)nc12] |
| H₄Noneu-npa | 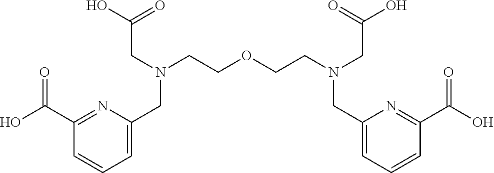 [SMILES: O=C(O)CN(CCOCCN(CC(=O)O)Cc1cccc(C(=O)O)n1)Cc1cccc(C(=O)O)n1] |
| mertiatide | 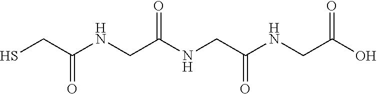 [SMILES: O=C(O)CNC(=O)CNC(=O)CNC(=O)CS] |
| DMSA | 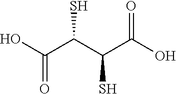 |
| HYNIC | 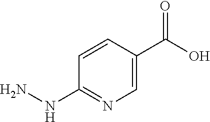 [SMILES: NNc1ccc(C(=O)O)cn1] |
| BOPTA | 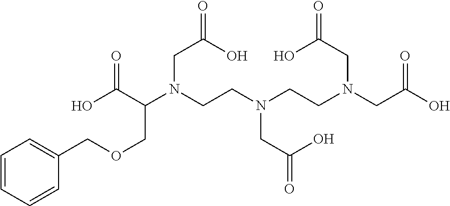 [SMILES: O=C(O)CN(CCN(CC(=O)O)CC(=O)O)CCN(CC(=O)O)C(COCc1ccccc1)C(=O)O] |

Beyond the chelators provided in Tables 3-7, this application covers structurally modified chelators (e.g., containing different or additional substituents, replacement of chelating atoms, etc.) provided that the structurally modified chelators have the same properties as those listed above.

Additional chelators that may be incorporated in HER2-targeting compounds of the present disclosure are described in Holik, et al. “The Chemical Scaffold of Theranostic Radiopharmaceuticals: Radionuclide, Bifunctional Chelator, and Pharmacokinetics Modifying Linker.” Molecules 27.10 (2022): 3062 and Kostelnik, et al. “Radioactive main group and rare earth metals for imaging and therapy.” Chemical Reviews 119.2 (2018): 902-956, both of which are incorporated by reference in their entireties.

In some embodiments of the HER2-targeting compounds described herein, the compound comprises at least one chelator (e.g., at least one M), and the chelator is selected from DOTA, DOTAGA, NODAGA, AAZTA, NOTA, and p-SCN-Bn-DOTA. In further embodiments, the chelator is selected from DOTA, DOTAGA, NODAGA, and AAZTA. In still further embodiments, the chelator is selected from DOTA, DOTAGA, and NODAGA. In certain embodiments, the chelator is DOTA. In certain embodiments, the chelator is NODAGA. In certain embodiments, the chelator is DOTAGA.

In various embodiments, M is a cyclic chelating agent.

In further embodiments, M is a cyclic chelating agent selected from:

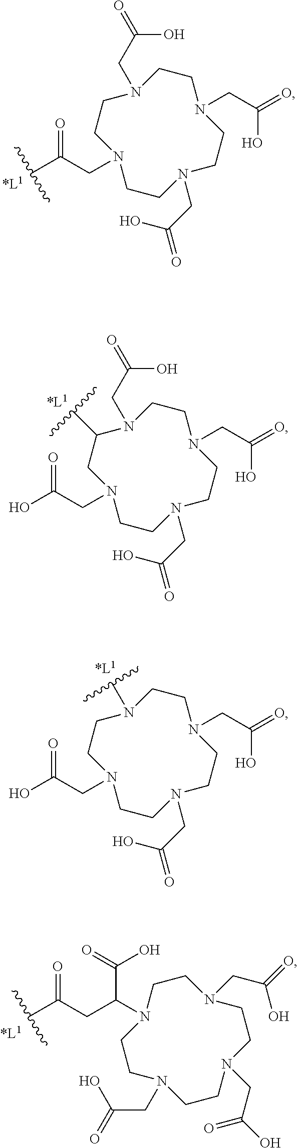 [SMILES: *C(=O)CN1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *N1CCN(CC(=O)O)CCN(CC(=O)O)CCN(CC(=O)O)CC1 ; *C(=O)CC(C(=O)O)N1CCN(CC(=O)O)CCN(CC(=O)O)CCN(CC(=O)O)CC1]

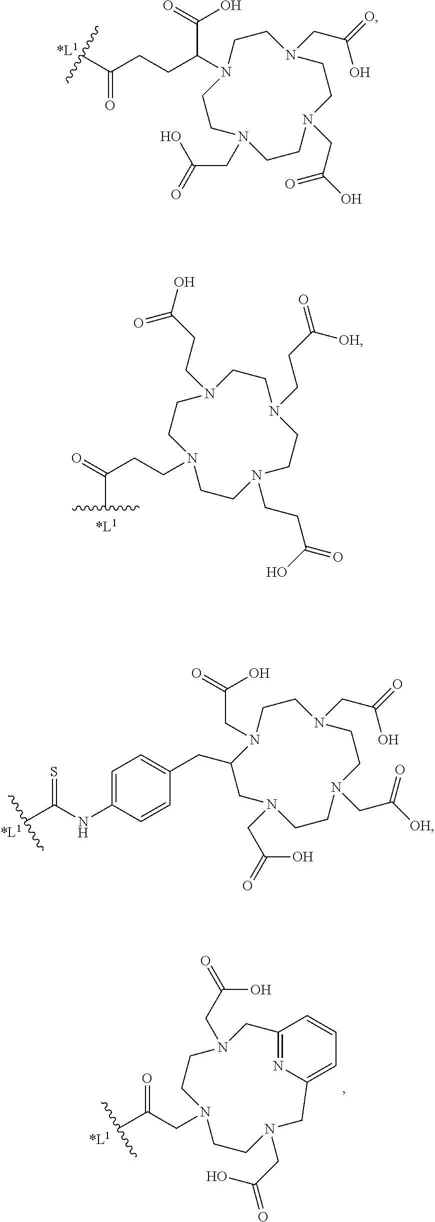 [SMILES: *C(=O)CCC(C(=O)O)N1CCN(CC(=O)O)CCN(CC(=O)O)CCN(CC(=O)O)CC1 ; *C(=O)CCN1CCN(CCC(=O)O)CCN(CCC(=O)O)CCN(CCC(=O)O)CC1 ; *C(=S)Nc1ccc(CC2CN(CC(=O)O)CCN(CC(=O)O)CCN(CC(=O)O)CCN2CC(=O)O)cc1 ; *C(=O)CN1CCN(CC(=O)O)Cc2cccc(n2)CN(CC(=O)O)CC1]

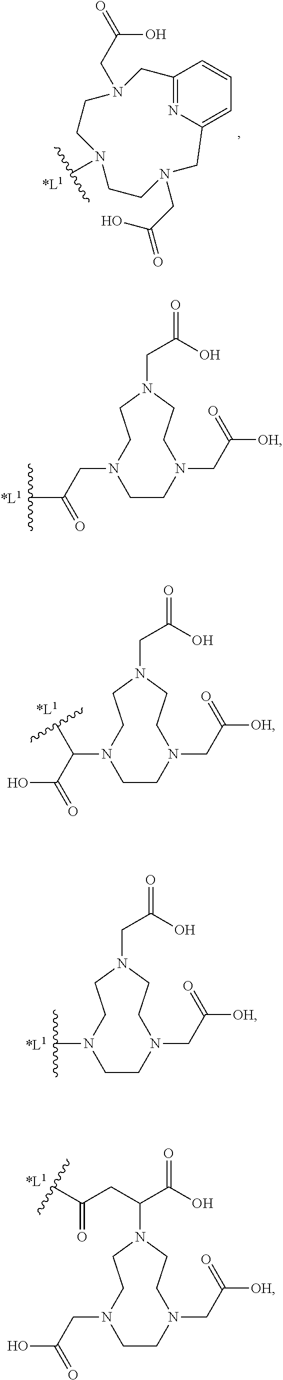

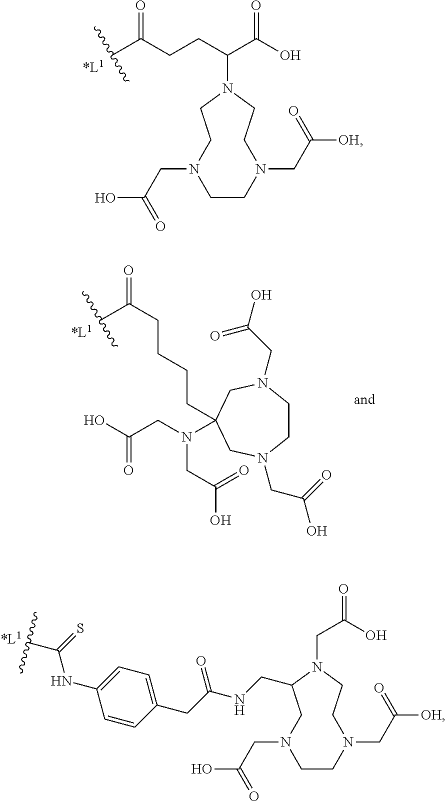 [SMILES: *C(=O)CCC(C(=O)O)N1CCN(CC(=O)O)CCN(CC(=O)O)CC1 ; *C(=O)CCCCC1(N(CC(=O)O)CC(=O)O)CN(CC(=O)O)CCN(CC(=O)O)C1 ; *C(=S)Nc1ccc(CC(=O)NCC2CN(CC(=O)O)CCN(CC(=O)O)CCN2CC(=O)O)cc1]

wherein *L¹ indicates the point of attachment to L¹ and wherein the cyclic chelating agent is optionally radiolabeled with a radionuclide.

In further embodiments, M is a cyclic chelating agent selected from:

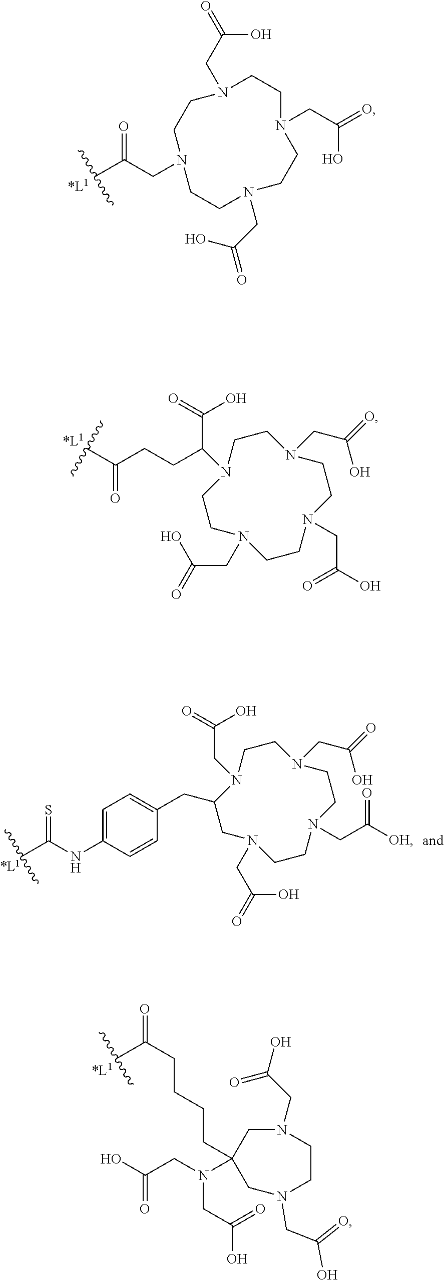 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=S)Nc1ccc(CC2CN(CC(=O)O)CCN(CC(=O)O)CCN(CC(=O)O)CCN2CC(=O)O)cc1 ; *C(=O)CCCCC1(N(CC(=O)O)CC(=O)O)CN(CC(=O)O)CCN(CC(=O)O)C1]

wherein *L¹ indicates the point of attachment to L¹ and wherein the cyclic chelating agent is optionally radiolabeled with a radionuclide.

In some embodiments the chelator or chelator residue includes any additional bridging atom or bridging moiety for attaching the chelator to L¹ or the cyclic peptide P. In a nonlimiting example, bridging atoms or bridging moieties include N, O, S, C(O), or combinations thereof.

The chelator may be covalently attached to L¹ or the cyclic peptide (P) via an attachment at any site on the chelator. For example, a covalent attachment may be formed between a functional group of a functionalized heteroatom, a heteroatom, or an alkyl, aryl, or cycloalkyl group separating the heteroatoms of the chelator to a suitable site on the linker or cyclic peptide.

In an embodiment, when the chelator (e.g., M) is substituted by a carboxy (—COOH) functional group, that functional group can serve as a handle for covalent attachment to L¹ or the cyclic peptide (P). In embodiments, the carboxy-L¹ or carboxy-P attachment point is an amide (—C(O)N(R)—) moiety. In non-limiting embodiments, the language “M is bound to the —(CH₂CH₂O)_{z}— via an amide bond,” covers, for example,

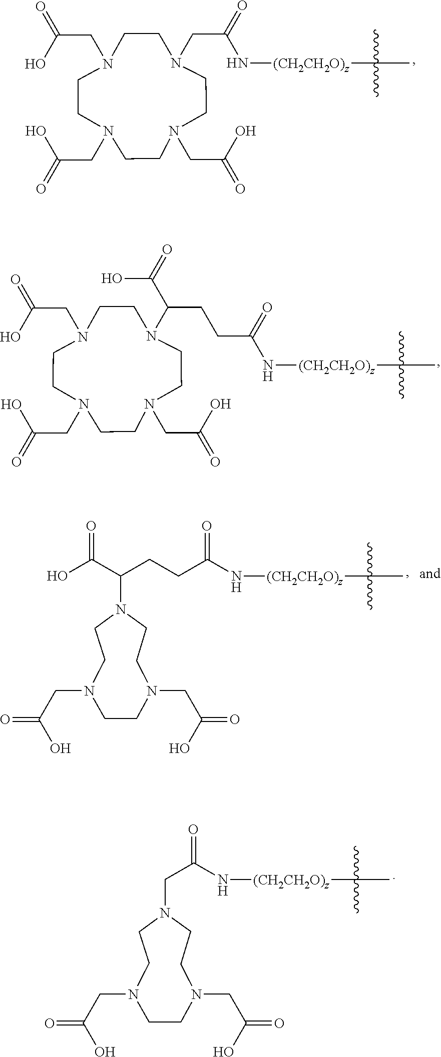

In one or more embodiments of the HER2-targeting compounds described herein (e.g., a compound of formula (I), (Ia), (Ib), (Ic) and (Id), or a pharmaceutically acceptable salt or solvate thereof), the chelator is attached to L¹, which is connected to a side chain of any amino acid of the cyclic peptide. In certain embodiments, the chelator is attached to L¹, which is connected to a side chain of A¹, A³, or A¹⁰. In certain embodiments, the chelator is attached to L¹, which is connected to a side chain of A¹.

In some embodiments, L¹ is a bond, and A¹, L¹, and M, together, have the structure:

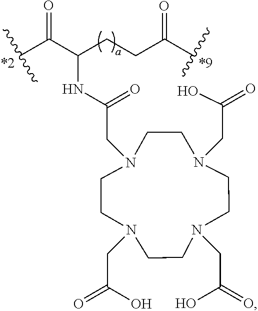

wherein M is optionally radiolabeled with a radionuclide.

In some embodiments, L¹ is a linker of formula (L¹-II), and A¹, L¹, and M, together, have the structure:

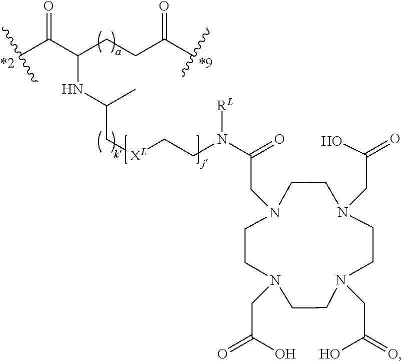

wherein M is optionally radiolabeled with a radionuclide.

In some embodiments, L¹ is a bond, and A¹⁰, L¹, and M, together, have the structure:

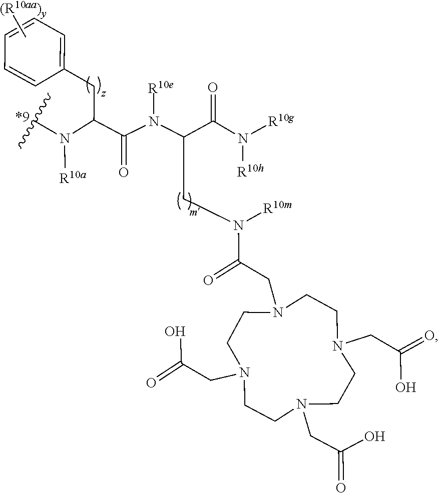

wherein:
R^{10m} is selected from H and C₁₋₃-alkyl; and
m′ is 1, 2, 3, 4, 5, or 6,
wherein M is optionally radiolabeled with a radionuclide.

When the chelator is covalently conjugated to L¹ or the cyclic peptide, a chemical substitution or reaction occurs to allow for covalent attachment to the linker or the cyclic peptide. The covalent attachment may be formed via any chemical substitution or reaction known in the art suitable for forming the given attachment. In some embodiments, one or more bonds to a hydrogen atom of the chelator are replaced by a bond to the linker or the cyclic peptide. In some embodiments, a π-bond, for example, of a double or triple bond, between two atoms is replaced by a bond from one of the two atoms to the linker or the cyclic peptide, wherein the other of the two atoms includes a new bond, for example, to a hydrogen (such as a reaction of an amine with an isocyanate (—NCS) to yield a thiourea bond or a reaction of maleimide and a thiol to produce a thioether bond). In some embodiments, a carboxylic acid or activated carboxylic acid is reacted with an amine of the cyclic peptide or linker to form an amide bond. Accordingly, one of skill in the art would recognize the various ways in which a chelator can be covalently attached to the linker or the cyclic peptide of any of the formulae presented herein.

### Radionuclides

In some embodiments of the HER2-targeting compounds described herein, M is a radionuclide. In some embodiments of the HER2-targeting compounds described herein, M is chelator radiolabeled with a radionuclide. Accordingly, in various embodiments, the HER2-targeting compounds may be radiolabeled with a diagnostically and/or therapeutically active radionuclide, thus providing a platform for imaging and radiotherapy targeting HER2. As used herein “radionuclide” (which also may be referred to those skilled in the art as radioactive atoms, radioactive elements, radioactive isotopes, or radioisotopes) is any unstable form of a chemical element that releases radiation. In some embodiments, the radionuclide releases or emits β-particles. In some embodiments, the radionuclide releases or emits α-particles. In some embodiments, the radionuclide releases or emits β-particles and α-particles.

The HER2-targeting compounds of the present disclosure may be radiolabeled with a radionuclide at any site of the HER2-targeting peptide. For example, in embodiments in which the HER2-targeting peptide is conjugated directly to a radionuclide, the radionuclide may by covalently attached to the HER2 targeting peptide. In other embodiments in which the HER2-targeting peptide is conjugated directly to a radionuclide, such conjugation may rely on ionic interactions, thereby forming a peptide-radionuclide salt. In some embodiments, when the HER2-targeting peptide is conjugated to a chelator, the HER2-targeting peptide may be radiolabeled via chelation of the radionuclide to the chelator. Chelation of a radionuclide to a chelator may be depicted using solid single bonds, dashed single bonds, or a combination thereof. For example, the chelation of ⁶⁸Ga to DOTA can be depicted below with solid single bonds or dashed single bonds. In some embodiments, the charge may also be indicated. For example, when a radionuclide is chelated to a chelating agent, each of the groups chelating the radionuclide may have a negative charge and the radionuclide being chelated may have an opposing positive charge. Such bonds and charges may be depicted herein as follows:

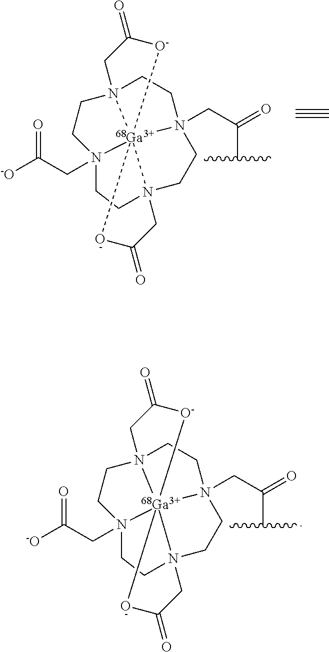

In the case of ²²⁵Ac, such bonds and charges may be illustrated (non-limiting) as follows:

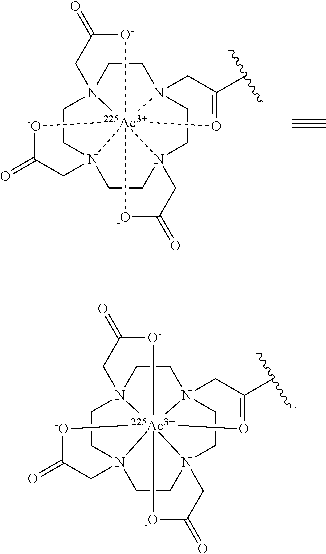

The radionuclide may be a therapeutic radionuclide, diagnostic radionuclide, or both. Suitable radionuclides include, but are not limited to, auger-electron emitting radionuclides, β-emitting (beta or beta-minus-emitting) radionuclides, and α-emitting (alpha-emitting) radionuclides. The selection of the type of radionuclide may depend on the use of the HER2-targeting compound. As will be appreciated by the skilled artisan, several factors may be considered when selecting a radionuclide for use in a HER2-targeting compound, such as, for example, the half-life, the linear energy transfer, the imaging capabilities, and the emission range in tissue. For example, b-emitting radionuclides typically have a longer emission range in tissue (e.g., 1-5 mm) and emit photons in an energy range that is easily imaged, and as such, they may be selected for use in a HER2-targeting compound being used for therapeutic, diagnostic, or theragnostic purposes. On the other hand, α-emitting radionuclides have a shorter emission range in tissue (e.g., 50-100 mm) and a high potency due to the amount of energy deposited per path length traveled (i.e., linear energy transfer), which is approximately 400 times greater than that of electrons. Thus, α-emitting radionuclides may be selected for therapeutic uses in which high potency of the radionuclide is desired.

Accordingly, in some embodiments, the radionuclide is an α-emitting radionuclide. In other embodiments, the radionuclide is a β-emitting radionuclide. In yet other embodiments, the radionuclide is an auger-electron emitting radionuclide.

The radionuclide may be a therapeutic radionuclide, an imaging radionuclide, or both. Accordingly, suitable radionuclides may have a decay energy sufficient for therapeutic and/or diagnostic use. In some embodiments, the decay energy of the radionuclide ranges from about 10 keV to about 6,000 keV. In some embodiments, the decay energy of the radionuclide is between 100 and 1,000 keV. Exemplary radionuclides that may be used to radiolabel the present HER2-targeting compounds are described in Sgouros et al. “Radiopharmaceutical therapy in cancer: clinical advances and challenges.” Nature reviews Drug discovery 19.9 (2020): 589-608, which is incorporated herein by reference in its entirety.

In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th. In some embodiments, the radionuclide is ⁶⁶Ga, ⁶⁷Ga, ⁶⁸Ga, ⁶⁴Cu, ¹⁷⁷Lu, or ²²⁵Ac. In some embodiments, the radionuclide is ¹¹¹In, ^{99m}Tc, ⁶⁸Ga, ⁶⁴Cu, ⁸⁹Zr, ¹²³I, ¹²⁴I, ¹⁸F, ⁹⁰Y, ¹⁷⁷Lu, ¹³¹I, ²²⁵Ac, ²¹¹At, or ²²⁷Th.

In some embodiments, the radionuclide is ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th.

In certain embodiments, the radionuclide is ¹⁷⁷Lu. In certain embodiments, the radionuclide is ²²⁵Ac. In certain embodiments, the radionuclide is ¹⁶¹Tb. In certain embodiments, the radionuclide is ⁶⁸Ga. In other embodiments, the radionuclide is ¹¹¹In.

In some embodiments, the radionuclide is a radiohalogen, e.g., ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I and ²¹¹At. In some embodiments, the radiohalogen is ¹⁸F. When the radionuclide is a radiohalogen, the term radiohalogen includes complexes that make the radiohalogen suitable for covalent attachment to the linker or the cyclic peptide or for chelation or complex formation with the chelator. Such complexes contemplated under the term radiohalogen include Si-¹⁸F, B-¹⁸F, and Al-¹⁸F.

In some embodiments, the radiohalogen is connected directly to the cyclic peptide or the linker. For example, ¹³¹I and ¹⁸F (or any other radiohalogen) can be substituted at any position of the linker or the cyclic peptide suitable for substitution with a halo group. In some embodiments, the radiohalogen is ¹⁸F. In some embodiments of the cyclic peptide (P), or compound comprising cyclic peptide (P): A¹⁰ is selected from:

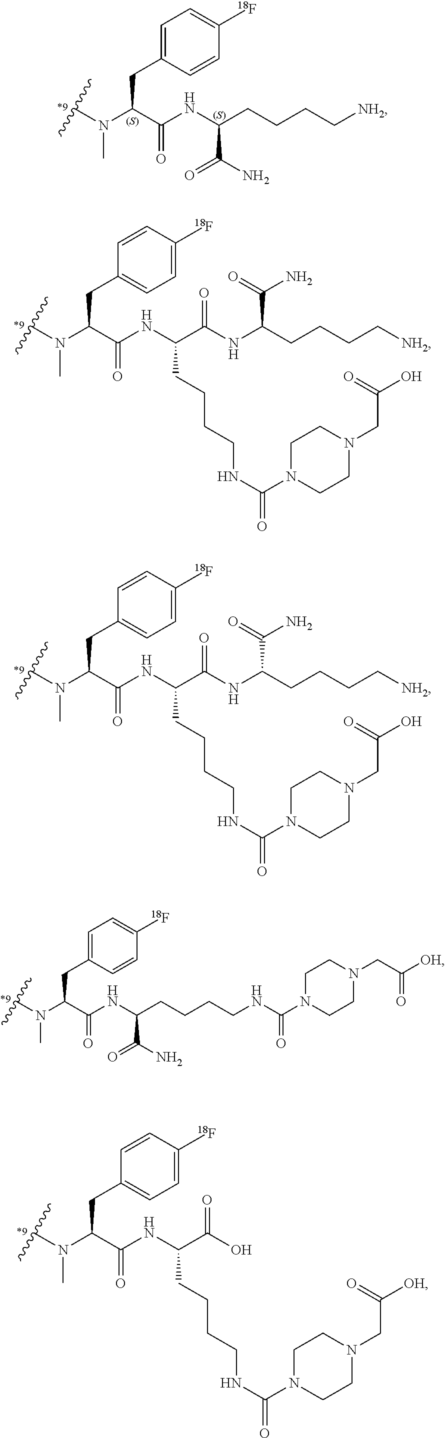

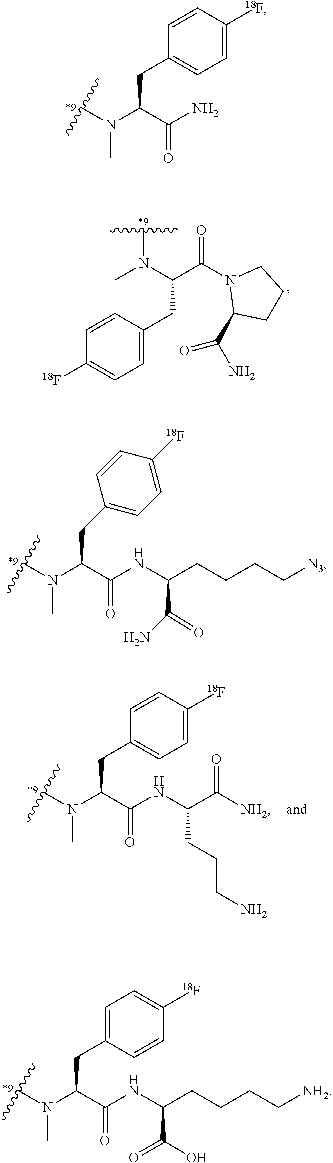

In some embodiments, when a radiohalogen is connected directly to the cyclic peptide or the linker, the chelator is absent.

In some embodiments, when the radiohalogen is ¹⁸F, it may be attached to L¹ via a prosthetic group and L¹ is a linker attached to a side chain of an amino acid of the cyclic peptide (P). In a further embodiment, M (when ¹⁸F) and L¹ (wherein L¹ is a bond or linker) together have the structure:

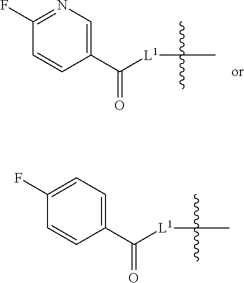

In some embodiments, M, L¹, and A¹⁰, together, have one of the following structures:

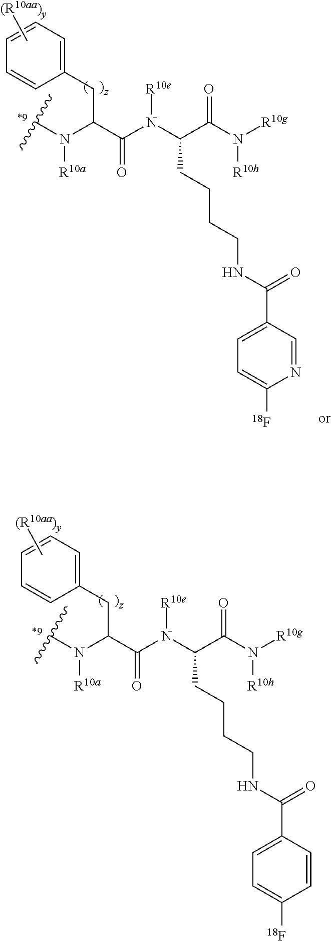

wherein *9 indicates the point of attachment to A⁹, and wherein the variables are defined elsewhere herein.

Alternatively, in some embodiments, the radiohalogen forms a complex with the chelator. For example, a radiohalogen complex such as, for example, Si-¹⁸F, B-¹⁸F, and Al-¹⁸F may chelate to a chelator of the presently described HER2-targeting compounds. One of ordinary skill in the art would understand that various radiohalide ions and metal or nonmetal elements may be combined to form a radiohalogen complex. Any such radiohalogen complex may be chelated to a chelator of the HER2-targeting compounds. In some embodiments, Si-¹⁸F is chelated to a chelator. In some embodiments, B-¹⁸F is chelated to a chelator. In some embodiments, Al-¹⁸F is chelated to a chelator.

The HER2-targeting compound of the present disclosure may be radiolabeled with one radionuclide or to more than one radionuclide. In some embodiments, the HER2-targeting compound is radiolabeled with one radionuclide. In some embodiments, the HER2-targeting compound is radiolabeled with more than one radionuclide. For example, the HER2-targeting compound made be radiolabeled with one radionuclide via complexation to the chelator and to another radionuclide via a direct covalent attachment to the linker, cyclic peptide, or the chelator. In some embodiments, a first radionuclide is chelated to the chelator and a second radionuclide is covalently attached to the cyclic peptide. In other embodiments, a first radionuclide is chelated to the chelator and a second radionuclide is covalently attached to the linker. In yet other embodiments, a first radionuclide is chelated to the chelator and a second radionuclide is covalently attached to the chelator.

In embodiments in which the HER2-targeting compound is radiolabeled with more than one radionuclide, each of the radionuclides may be the same or different. In some embodiments, two of the same radionuclides are used to radiolabel the HER2-targeting compound of the present disclosure. In some embodiments, two different radionuclides are used to radiolabel the HER2-targeting compound of the present disclosure. In some embodiments, the HER2-targeting compound is radiolabeled with a diagnostic radionuclide and a therapeutic radionuclide. In some embodiments, the HER2-targeting compound is radiolabeled with a radiohalogen and a radionuclide other than a radiohalogen. In some embodiments, the HER2-targeting compound is radiolabeled with a first radiohalogen and a second radiohalogen, where the first radiohalogen is different from the second radiohalogen.

In some embodiments, the radionuclide is a therapeutically active radionuclide. Suitable therapeutically active radionuclides include, but are not limited to, ⁶⁷Cu, ¹⁸⁶Re, ¹⁸⁸Re, ⁹⁰Y, ¹⁷⁷Lu, ¹⁶¹Tb, ¹⁵³Sm, ²¹³Bi, ¹³¹I, ¹⁴⁹Tb, ⁴⁷Sc, ²²⁵Ac, ²¹²Pb, ²¹¹At, ²²³Ra, ²²⁷Th, and ²²⁶Th. In some embodiments, the radionuclide is a therapeutically active radionuclide selected from ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, and ²²⁷Th. In particular embodiments, the radionuclide is a therapeutically active radionuclide selected from ⁹⁰Y, ¹⁷⁷Lu, ¹³¹I, ²²⁵Ac, ²¹¹At, and ²²⁷Th. In a particular embodiment, the therapeutically active radionuclide is ¹⁷⁷Lu. In a particular embodiment, the therapeutically active radionuclide is ²²⁵Ac.

Alternatively, in some embodiments, the radionuclide is a diagnostically active radionuclide (a radionuclide suitable for imaging as, e.g., by PET). Suitable diagnostically active radionuclides include, but are not limited to, ¹¹¹In, ^{99m}Tc, ^{94m}Tc, ⁶⁷Ga, ⁶⁸Ga, ²⁰³Pb, ⁶⁴Cu, ⁸⁶Y, ⁸⁹Zr, ⁵¹Mn, ⁵²Mn, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, and ²⁰¹Tl. In some embodiments, the radionuclide is a diagnostically active radionuclide selected from ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, and ⁴³Sc. In particular embodiments, the radionuclide is a diagnostically active radionuclide selected from ¹¹¹In, ^{99m}Tc, ⁶⁸Ga, ⁶⁴Cu, ⁸⁹Zr, ¹²³I, ¹²⁴I, and ¹⁸F. In a particular embodiment, the diagnostically active radionuclide is ⁶⁸Ga. In another particular embodiment, the diagnostically active radionuclide is ¹⁸F. In another particular embodiment, the diagnostically active radionuclide is ⁶⁴Cu. In another particular embodiment, the diagnostically active radionuclide is ¹¹¹In.

In some embodiments, the radionuclide coordinated to or covalently attached to the HER2-targeting compound described herein is stable in vivo. Stable radionuclides may have a half-life that allows for therapeutic and/or diagnostic medical use. For example, the radionuclide may have a half-life from about 10 minutes to about 50 days. In some embodiments, the radionuclide has a half-life between 1 hour and 20 days. In some embodiments, the radionuclide has a half-life between 1 day and 10 days.

In some embodiments, the radionuclide is detectable by positron emission spectroscopy (PET), positron emission tomography and computerized tomography (PET/CT), or single photon emission computed tomography (SPECT).

In some embodiments, the radionuclide is covalently attached directly to the cyclic peptide or the linker of the HER2-targeting compound. In some embodiments, the radionuclide is coordinated to the chelating ligand of the HER2-targeting compound. Various radionuclides may be complexed to various chelators disclosed herein. One of ordinary skill in the art would recognize the appropriate selection of chelator and radionuclide for any use or method contemplated by the instant disclosure.

### Additional Embodiments of Formulae (I), (La), (Ib), (Ic) and (Id)

As previously described, the HER2-targeting compounds of the present disclosure may be compounds of formula (I), (Ia), (Ib), (Ic) or (Id):

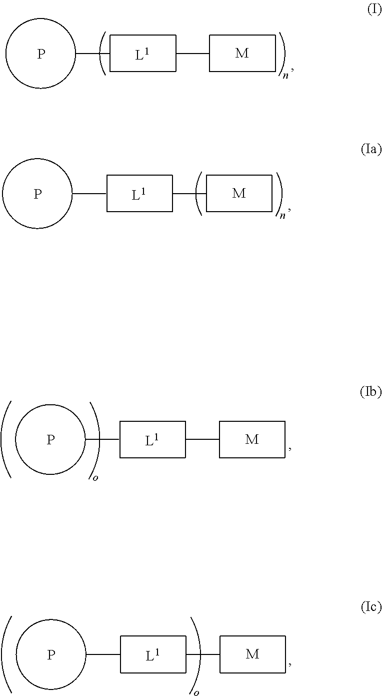

or pharmaceutically acceptable salts or solvates thereof, wherein P, L¹ and M are as previously described and n and o are each independently 1, 2, 3, or 4.

It is conceived herein that the HER2-targeting compound may include 1, 2, 3, or 4 P, P-L¹ moieties, M, or M-L¹ moieties. In some embodiments, the HER2-targeting compound includes 1, 2, 3, or 4 cyclic peptides, 1, 2, or 3, cyclic peptides, or 1 or 2 cyclic peptides. In some embodiments, the HER2-targeting compound includes 1, 2, 3, or 4 P-L¹ moieties, 1, 2, or 3, P-L¹ moieties, or 1 or 2 P-L¹ moieties. In some embodiments, the HER2-targeting compound includes 1, 2, 3, or 4 M moieties, 1, 2, or 3, M moieties, or 1 or 2 M moieties. In some embodiments, the HER2-targeting compound includes 1, 2, 3, or 4 m-L¹ moieties, 1, 2, or 3, M-L¹ moieties, or 1 or 2 M-L¹ moieties.

In embodiments in which the HER2-targeting peptide includes at least two cyclic peptides, each cyclic peptide can be the same or different. In embodiments in which the HER2-targeting peptide includes at least two P-L¹ moieties, each cyclic peptide and each L¹ may be independently selected and can be the same or different. In embodiments in which the HER2-targeting peptide includes at least two M moieties, each M moiety can be the same or different. In embodiments in which the HER2-targeting peptide includes at least two M-L¹ moieties, each M moiety and each L¹ may be independently selected and can be the same or different.

In some embodiments, the compounds of formulae (I), (Ia), (Ib), and (Ic) are compounds of formulae (I-i), (Ia-i), (Ib-i) and (Ic-i):

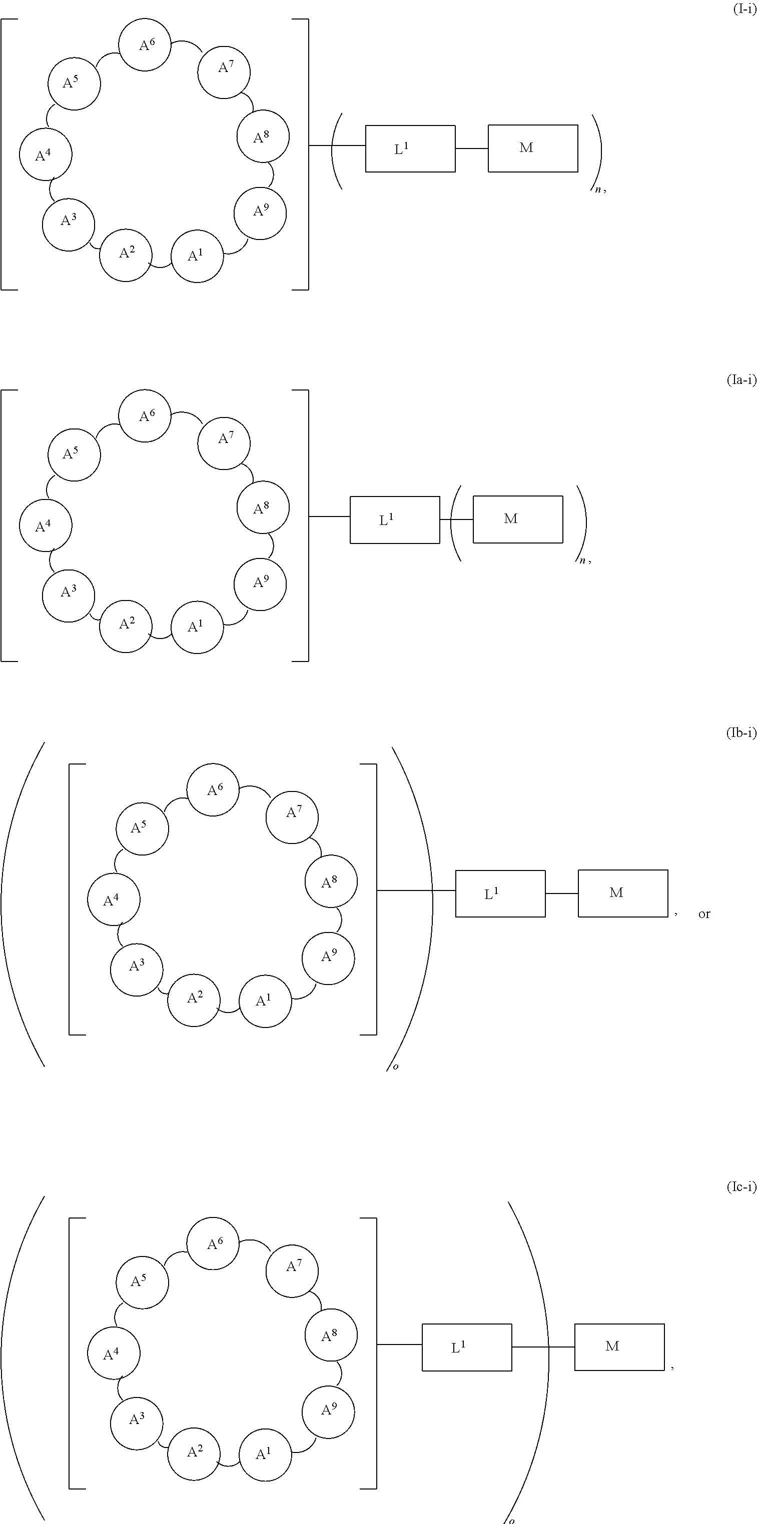

wherein A¹-A⁹ are as defined elsewhere herein,
L¹ is, independently at each occurrence, selected from the group consisting of a bond and a linker;
M is, independently at each occurrence, selected from the group consisting of an imaging agent, a chelating agent, and a radionuclide the chelating agent is optionally radiolabeled with a radionuclide;
n is 1, 2, 3, or 4;
o is 1, 2, 3, or 4;
and wherein any of A¹-A⁹ and L¹ is optionally substituted with an albumin binder. In certain embodiments, the compound of formula (Ia) or formula (Ia-i), or a pharmaceutically acceptable salt or solvate thereof, is Example D6.

In certain embodiments, the HER2-targeting compound is compound of formula (I):

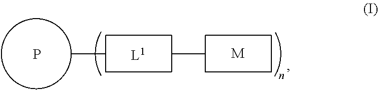

or a pharmaceutically acceptable salt or solvate thereof, wherein is 1. For example, in some embodiments, the compound of formula (I) is a compound of formula (Id):

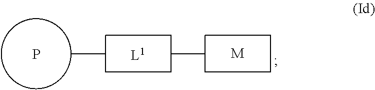

or a pharmaceutically acceptable salt or solvate thereof,
wherein:
P is a cyclic (HER2-targeting) peptide;
L¹ is a bond or linker; and
M is a chelating agent optionally radiolabeled with a radionuclide;
and wherein the cyclic peptide, linker, chelating agent, and radionuclide are as disclosed elsewhere herein.

In some embodiments of the compound of formula (Id), or a pharmaceutically acceptable salt or solvate thereof, M is a chelator optionally radiolabeled with a radionuclide, wherein the chelator is selected from DOTA, DOTAGA, NODAGA, AAZTA, NOTA, and p-SCN-Bn-DOTA. In some embodiments of the compound of formula (Id), or a pharmaceutically acceptable salt or solvate thereof, M is a chelator optionally radiolabeled with a radionuclide, wherein the chelator is selected from DOTA, DOTAGA, and NODAGA. In some embodiments of the compound of formula (Id), or a pharmaceutically acceptable salt or solvate thereof, M is a chelator optionally radiolabeled with a radionuclide, wherein the chelator is DOTA.

In some embodiments of the compound of formula (Id), or a pharmaceutically acceptable salt or solvate thereof, M is a chelator that is not radiolabeled with a radionuclide. Alternatively, in some embodiments of the compound of formula (Id), or a pharmaceutically acceptable salt or solvate thereof, M is a chelator that is radiolabeled with a radionuclide.

In some embodiments, the compound of formula (Id) is a compound of formula (Id-i):

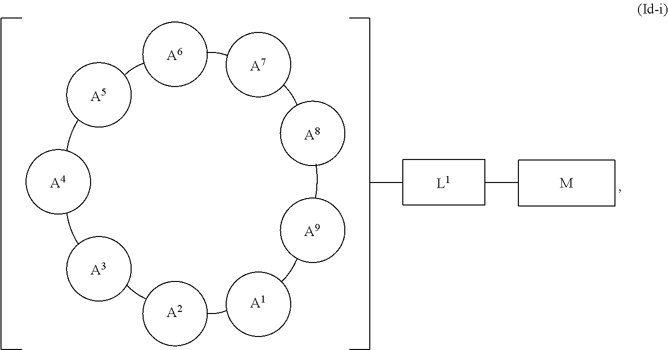

or a pharmaceutically acceptable salt or solvate thereof,
wherein A¹-A⁹ are as defined elsewhere herein, and L¹ is connected to the cyclic HER2-targeting peptide via a side chain of any one of A¹-A¹⁰ (wherein A¹⁰ is included in the definition of A⁹).

In some embodiments, the compound of formula (Id), or a pharmaceutically acceptable salt or solvate thereof, is a compound of formula (Id-ii):

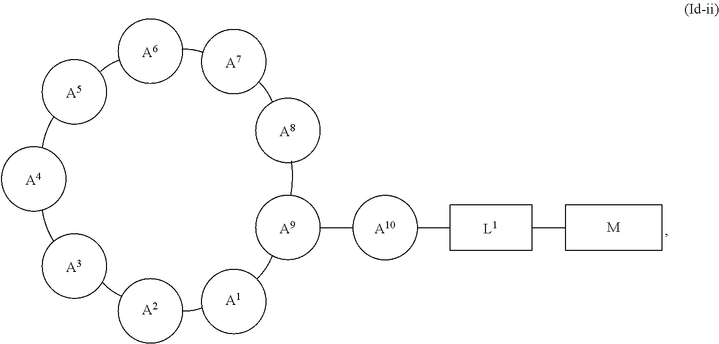

or a pharmaceutically acceptable salt or solvate thereof,
wherein L¹ is connected to the cyclic HIER2-targeting peptide via a side chain of A¹⁰. In some embodiments of the compound of formula (Id-ii), or a pharmaceutically acceptable salt or solvate thereof, L¹ is connected to the cyclic HER2-targeting peptide via a side chain NH₂ of A¹⁰.

In some embodiments, the compound of formula (Id) (and subgenera of formula (Id), e.g., formulae (Id-i) and (Id-ii)), or a pharmaceutically acceptable salt or solvate thereof, has the following definitions:
A¹ is a moiety of formula (A1-I):

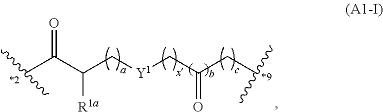

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
Y¹ is selected from a bond, C≡C, NH, NC₁₋₆-alkyl, O, and S;
a is 1, 2, or 3; and
b, c, and x′ are each independently 0 or 1; or
A¹ is substituted with a PEG chain, wherein A¹ and the PEG chain, together, have the structure of formula (A1-P):

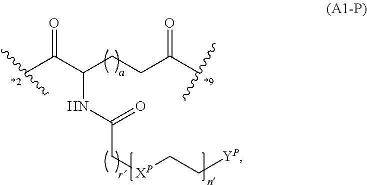

wherein:
X^{P} is selected from CH₂, N(R^{P}), and O;
Y^{P} is selected from H, —OH, —OC₁₋₆-alkyl, and —N(R^{P}) 2;
each R^{P} is independently selected from H and C₁₋₆-alkyl;
a is 1, 2, or 3;
n′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
r′ is 0, 1, 2, 3, or 4;
A² is a moiety of formula (A2-I):

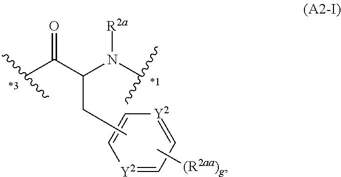

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2;
A³ is a moiety of formula (A3-I) or formula (A3-II):

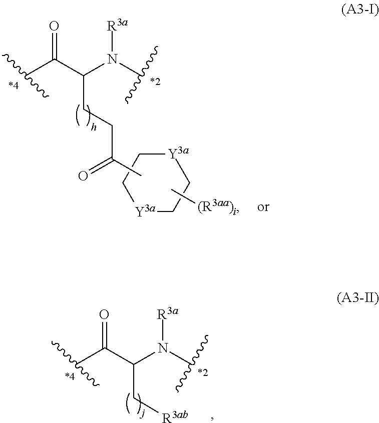

wherein:
R^{3a} is selected from H and C₁₋₃-alkyl;
each R^{3aa} is independently selected from —C(O)OH, —C₁₋₆-alkyl-C(O)OH, —C(O)NH₂, —NH₂, —NHC(O)C₁₋₆-alkyl, and —OC₁₋₆-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl;
Y³ is selected from a bond and NH;
each Y^{3a} is selected from NH and CH₂, provided that at least one Y^{3a} is NH;
h is 1, 2, 3, 4, 5, or 6;
i is 0, 1, or 2; and
j is 1, 2, 3, 4, 5, or 6;
A⁴ is a moiety of formula (A4-I):

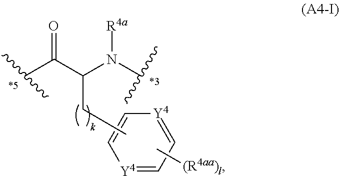

wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
k is 1, 2, 3, 4, 5, or 6; and
l is 0, 1, or 2;
A⁵ is a moiety of formula (A5-I) or formula (A5-II):

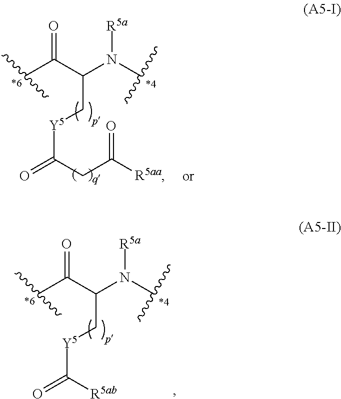

wherein:
Y⁵ is selected from O and NH;
R^{5a} is selected from H and C₁₋₃-alkyl;
R^{5aa} is selected from H, —OH, and —OC₁₋₆-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH; and
p′ and q′ are each independently 1, 2, 3, 4, 5, or 6;
A⁶ is a moiety of formula (A6-Ia):

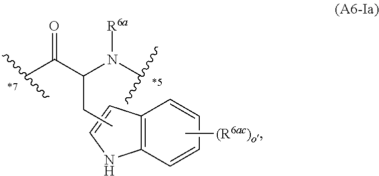

wherein:
R^{6a} is selected from H and C₁₋₃-alkyl;
R^{6ac} is selected from OH, C₁₋₆-alkyl, and —C(O)—C₁₋₆-alkyl; and
o′ is 0, 1, or 2;
A⁷ is a moiety of formula (A7-Ia):

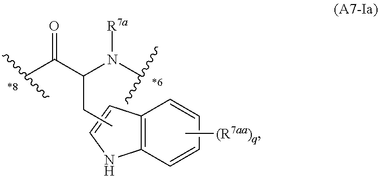

wherein:
R^{7a} is selected from H and C₁₋₃-alkyl;
each R^{7aa} is selected from —CN, halo, —NH₂, and —OH; and
q is 0, 1, or 2;
A⁸ is a moiety of formula (A8-I) or formula (A8-II):

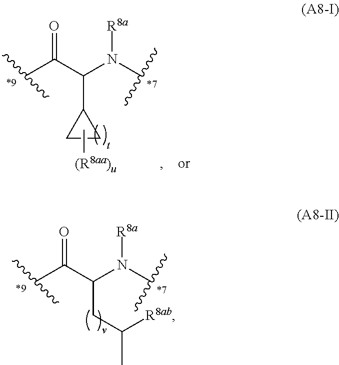

wherein:
R^{8a} is selected from H and C₁₋₃-alkyl;
R^{8aa} is selected from —CN, halo, —NH₂, and —OH;
R^{8ab} is selected from H, —CN, halo, —NH₂, and —OH;
t is 1 or 2;
u is 0, 1, or 2; and
v is 0, 1, 2, 3, or 4; and
A⁹ is a moiety of formula (A9-I):

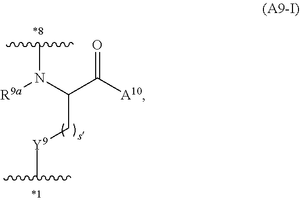

wherein:
Y⁹ is selected from C(O), NH, NC₁₋₆-alkyl, and S;
R^{9a} is selected from H and C₁₋₃-alkyl; and
s′ is 1 or 2; and
A¹⁰, L¹, and M, together, have the structure:

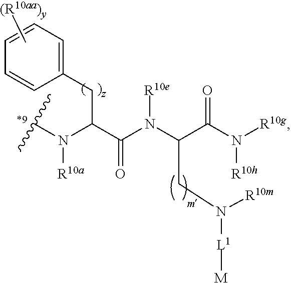

wherein:
R^{10a}, R^{10e}, and R^{10m} are each independently selected from H and C₁₋₃-alkyl;
R^{10g} and R^{10h} are each independently selected from H and C₁₋₆-alkyl;
each R^{10aa} is independently selected from C₁₋₆-alkyl, halo, —NH₂, —N₃, —OH, and —OC₁-6-alkyl;
y is 0, 1, or 2;
z is 1, 2, 3, 4, 5, or 6; and
m′ is 1, 2, 3, 4, 5, or 6,
wherein L¹ is a bond or a linker as defined elsewhere herein, and wherein M is a chelator optionally radiolabeled with a radionuclide.

In an embodiment, A¹ is a moiety of formula (A1-I), A³ is a moiety of formula (A3-II), A⁵ is a moiety of formula (A5-II), and A⁸ is a moiety of formula (A8-I):

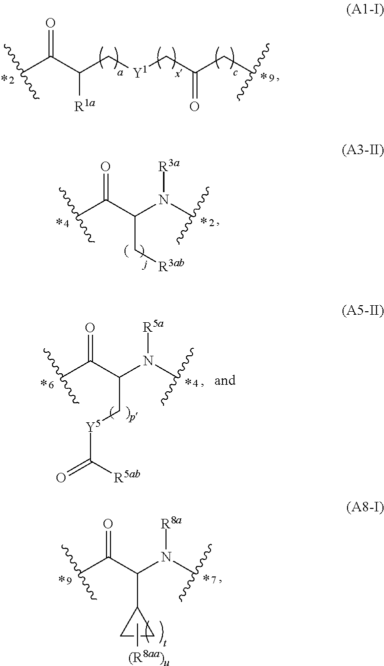

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
R^{8a} is selected from H and C₁₋₃-alkyl;
R^{8aa} is selected from —CN, halo, —NH₂, and —OH;
Y¹ is selected from a bond, C≡C, NH, NC₁₋₆-alkyl, O, and S;
Y⁵ is selected from O and NH;
a is 1, 2, or 3;
b, c, and x′ are each independently 0 or 1;
j is 1, 2, 3, 4, 5, or 6;
t is 1 or 2;
u is 0, 1, or 2; and
p′ is 1, 2, 3, 4, 5, or 6.

In another embodiment, A¹ is a moiety of formula (A1-I), A³ is a moiety of formula (A³-II), A⁵ is a moiety of formula (A5-II), and A⁸ is a moiety of formula (A8-II):

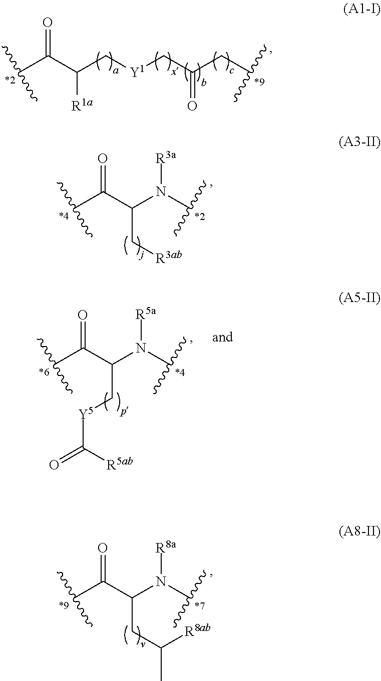

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
R^{8a} is selected from H and C₁₋₃-alkyl;
R^{8ab} is selected from H, —CN, halo, —NH₂, and —OH;
Y¹ is selected from a bond, C≡C, NH, NC₁₋₆-alkyl, O, and S;
Y⁵ is selected from O and NH;
a is 1, 2, or 3;
b, c, and x′ are each independently 0 or 1;
j is 1, 2, 3, 4, 5, or 6;
v is 0, 1, 2, 3, or 4; and
p′ is 1, 2, 3, 4, 5, or 6.

In yet another embodiment, A¹ is a moiety of formula (A1-P), A³ is a moiety of formula (A3-II), A⁵ is a moiety of formula (A5-II), and A⁸ is a moiety of formula (A8-I):

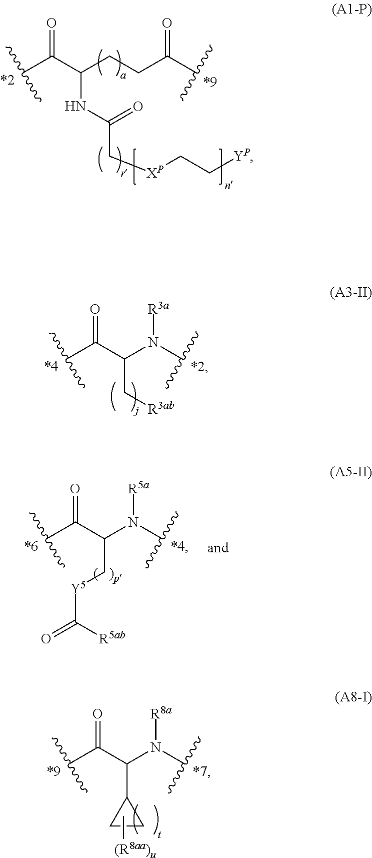

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
R^{8a} is selected from H and C₁₋₃-alkyl;
R^{8aa} is selected from —CN, halo, —NH₂, and —OH;
Y¹ is selected from a bond, C≡C, NH, NC₁₋₆-alkyl, O, and S;
Y⁵ is selected from O and NH;
a is 1, 2, or 3;
b, c, and x′ are each independently 0 or 1;
j is 1, 2, 3, 4, 5, or 6;
t is 1 or 2;
u is 0, 1, or 2; and
p′ is 1, 2, 3, 4, 5, or 6.

In still another embodiment, the compound of formula (Id), or a pharmaceutically acceptable salt or solvate thereof, is a compound of formula (Id-iia):

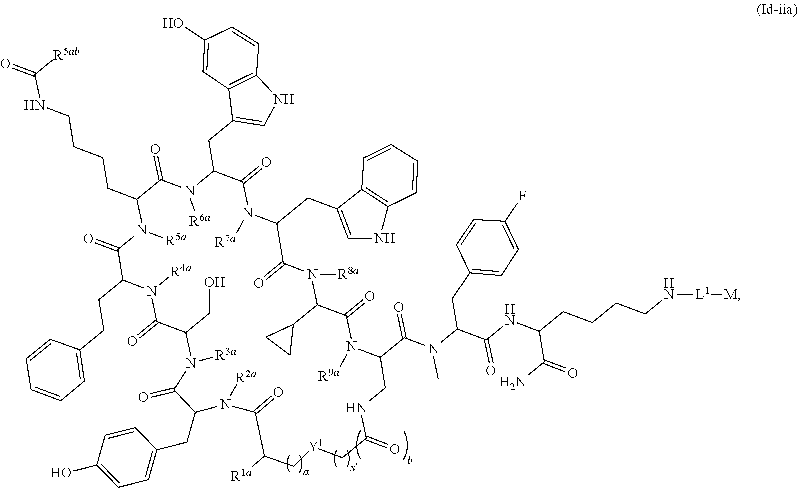

or a pharmaceutically acceptable salt or solvate thereof,
   wherein:
L¹ is a bond or a linker as defined elsewhere herein; and
M is a chelator optionally radiolabeled with a radionuclide, wherein M is bound to L¹ via an amide bond;
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
Y¹ is selected from a bond, C≡C, NH, NC₁₋₆-alkyl, O, and S;
R^{2a}, R^{3a}, R^{4a}, R^{5a}, R^{6a}, R^{7a}, R^{8a}, and R^{9a} are each independently selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
a is 1, 2, or 3; and
b and x′ are each independently 0 or 1.

In an embodiment, M is radiolabeled with ¹¹¹In.

In an embodiment, M is radiolabeled with ²²⁵Ac.

In an embodiment, M is radiolabeled with ⁶⁸Ga.

In an embodiment, M is radiolabeled with ¹⁷⁷Lu.

In an embodiment, the compound of formula (Id), or a pharmaceutically acceptable salt or solvate thereof, is a compound of formula (Id-iib):

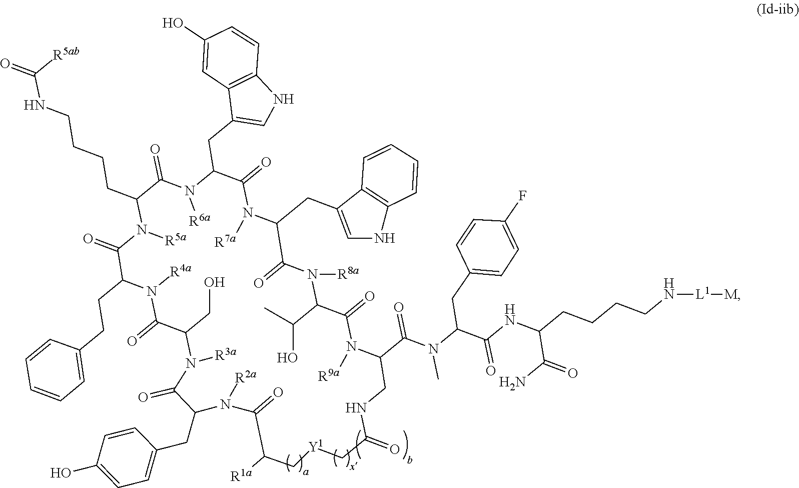

or a pharmaceutically acceptable salt or solvate thereof,
   wherein:
L¹ is a linker as defined elsewhere herein; and
M is a chelator optionally radiolabeled with a radionuclide, wherein M is bound to L¹ via an amide bond;
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl;
Y¹ is selected from a bond, C≡C, NH, NC₁₋₆-alkyl, O, and S;
R^{2a}, R^{3a}, R^{4a}, R^{5a}, R^{6a}, R^{7a}, R^{8a}, and R^{9a} are each independently selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
a is 1, 2, or 3; and
b and x′ are each independently 0 or 1.

In an embodiment, M is radiolabeled with ⁶⁸Ga or ¹⁷⁷Lu.

In another embodiment, the compound of formula (Id), or a pharmaceutically acceptable salt or solvate thereof, is a compound of formula (Id-iic):

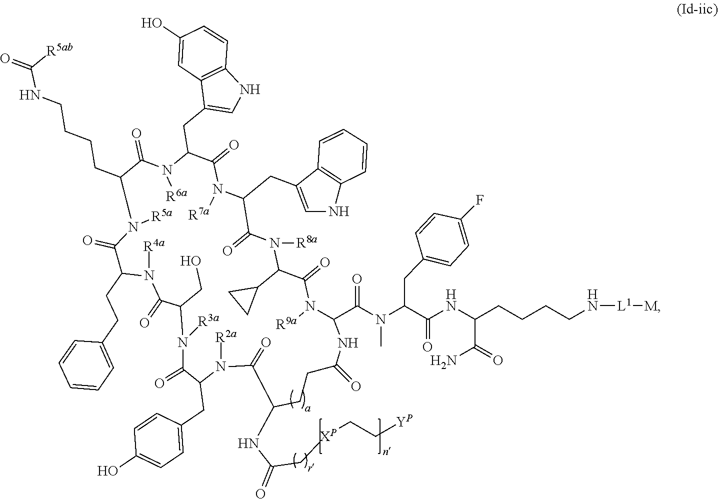

or a pharmaceutically acceptable salt or solvate thereof,
   wherein:
L¹ is a linker as defined elsewhere herein; and
M is a chelator optionally radiolabeled with a radionuclide, wherein M is bound to L¹ via an amide bond;
X^{P} is selected from CH₂, N(R^{P}), and O;
Y^{P} is selected from H, —OH, —OC₁₋₆-alkyl, and —N(R^{P}) 2;
each R^{P} is independently selected from H and C₁₋₆-alkyl;
R^{2a}, R^{3a}, R^{4a}, R^{5a}, R^{6a}, R^{7a}, R^{8a}, and R^{9a} are each independently selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
a is 1, 2, or 3;
n′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
r′ is 0, 1, 2, 3, or 4.

In an embodiment, M is radiolabeled with ⁶⁸Ga or ¹⁷⁷Lu.

In an embodiment of formula (Id-iia) and formula (Id-iib), or a pharmaceutically acceptable salt or solvate thereof,
R^{1a} is selected from H, —NH₂, —N(H)—C(O)—(C₁₋₆-alkyl), —NHC₁₋₆-alkyl, and —N(C₁₋₆-alkyl)₂, wherein the —N(H)—C(O)—C₁₋₆-alkyl of R^{1a} is optionally substituted with 1 substituent selected from —C(O)OH, —C(O)C₁₋₆-alkyl, and —C(O)C₁₋₆-alkyl-OH, 5-membered heteroaryl;
Y¹ is selected from a bond, O, and S; and
R^{5ab} is selected from H, C₁₋₆-alkyl, and C₁₋₆-haloalkyl, wherein the C₁₋₆-alkyl of R^{5ab} is optionally substituted with 1 substituent independently selected from —NH₂ and —OH.

In an embodiment of formula (Id-iic), or a pharmaceutically acceptable salt or solvate thereof,
R^{5ab} is selected from H, C₁₋₆-alkyl, and C₁₋₆-haloalkyl, wherein the C₁₋₆-alkyl of R^{5ab} is optionally substituted with 1 substituent independently selected from —NH₂ and —OH;
X^{P} is O;
Y^{P} is-OC₁₋₆-alkyl; and
n′ is 1, 2, 3, 4, 5, 6, 7, 8, 9, 10, 11, 12, 13, 14, or 15.

In an embodiment of formula (Id-iia), formula (Id-iib), and formula (Id-iic), or a pharmaceutically acceptable salt or solvate thereof, L¹ is a bond. In another embodiment of formula (Id-iia), formula (Id-iib), and formula (Id-iic), or a pharmaceutically acceptable salt or solvate thereof, M is selected from DOTA, DOTAGA, NODAGA, AAZTA, NOTA, and p-SCN-Bn-DOTA, wherein M is bound to L¹ via an amide bond. In yet another embodiment of formula (Id-iia), formula (Id-iib), and formula (Id-iic), or a pharmaceutically acceptable salt or solvate thereof, M is DOTA, wherein M is bound to L¹ via an amide bond.

In an embodiment, M is radiolabeled with ⁶⁸Ga or ¹⁷⁷Lu.

In an embodiment of formula (Id-iia), or a pharmaceutically acceptable salt or solvate thereof, is a compound of formula (Id-iia-i):

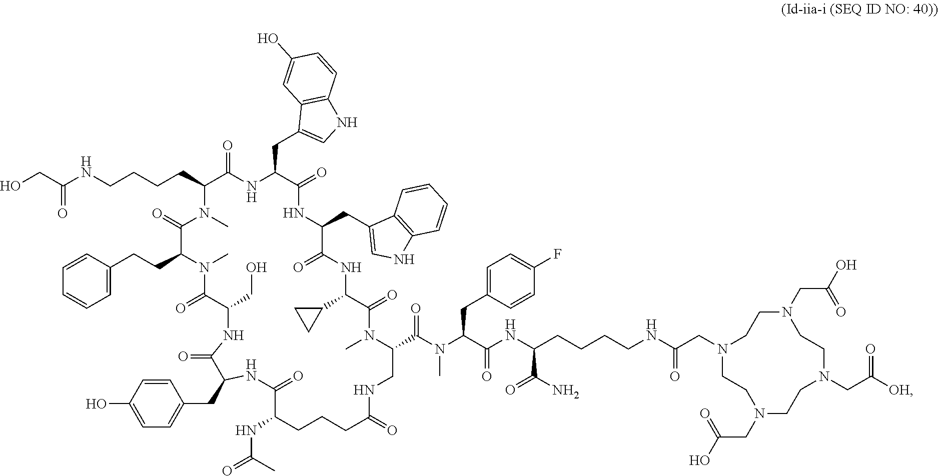 [SMILES: CC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(=O)CO)N(C)C(=O)[C@H](CCc2ccccc2)N(C)C(=O)[C@H](CO)NC(=O)[C@H](Cc2ccc(O)cc2)NC1=O]

which is optionally radiolabeled with a radionuclide defined elsewhere herein. In an embodiment, the compound of formula (Id-iia-i) is radiolabeled with a radionuclide. In a further embodiment, the radionuclide is ⁶⁸Ga, ¹¹¹In, ¹⁷⁷Lu, or ²²⁵Ac.

In yet another embodiment of formula (Id-iia), or a pharmaceutically acceptable salt or solvate thereof, is a compound of formula (Id-iia-ii):

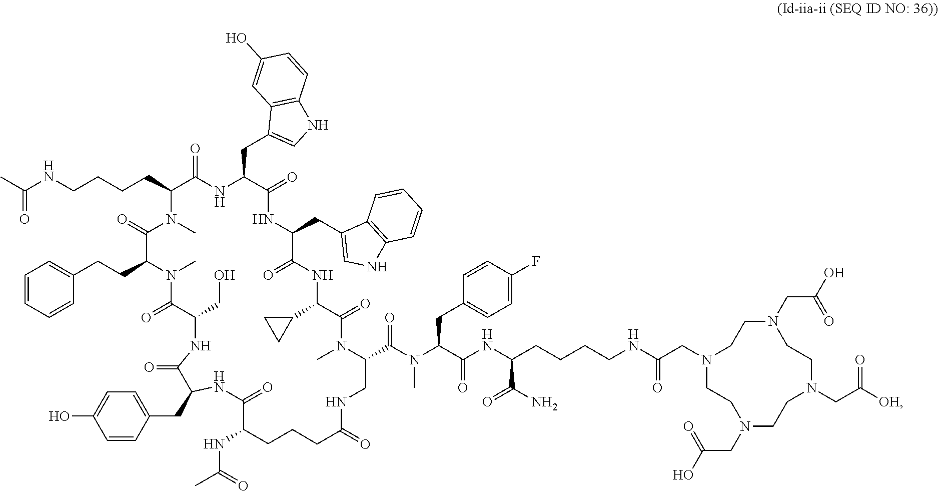 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-ii) is radiolabeled with a radionuclide. In a further embodiment, the radionuclide is ⁶⁸Ga, ¹¹¹In, ¹⁷⁷Lu, or ²²⁵Ac.

In still another embodiment of formula (Id-iia), or a pharmaceutically acceptable salt or solvate thereof, is a compound of formula (Id-iia-iii):

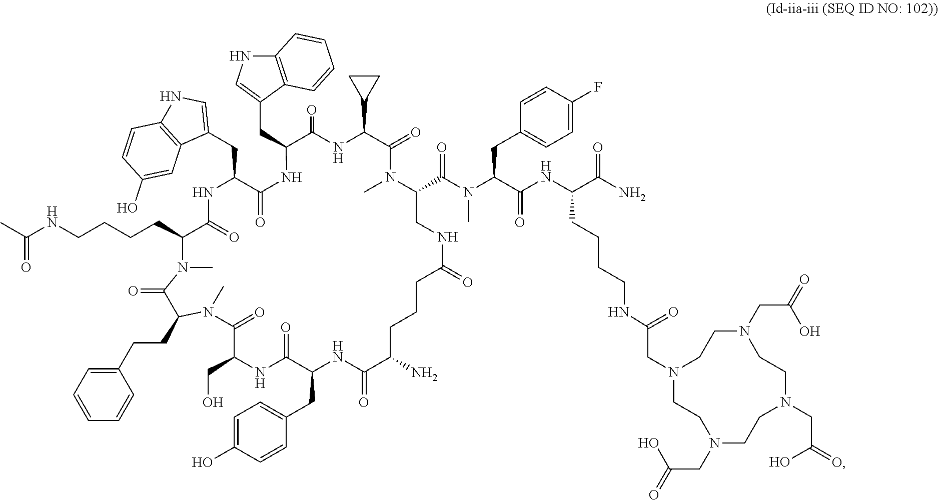 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](N)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-iii) is radiolabeled with a radionuclide. In a further embodiment, the radionuclide is ⁶⁸Ga, ¹¹¹In, ¹⁷⁷Lu, or ²²⁵Ac.

In an embodiment of formula (Id-iia), or a pharmaceutically acceptable salt or solvate thereof, is a compound of formula (Id-iia-iv):

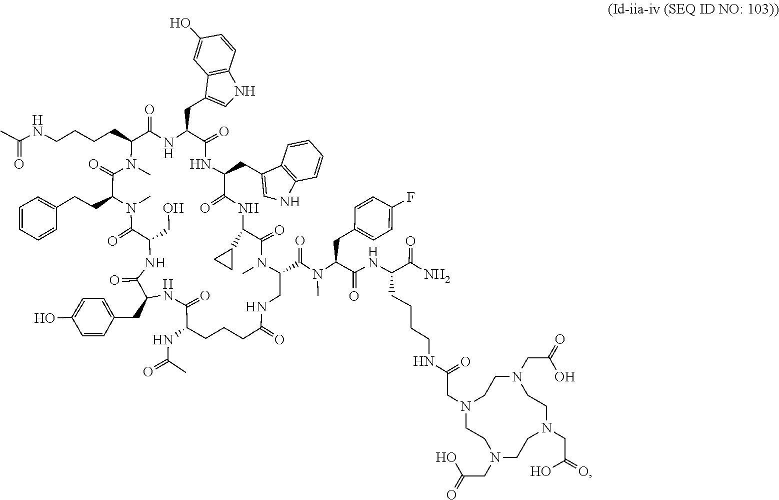 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-iv) is radiolabeled with a radionuclide. In a further embodiment, the radionuclide is ⁶⁸Ga, ¹¹¹In, ¹⁷⁷Lu, or ²²⁵Ac.

In another embodiment of formula (Id-iia), or a pharmaceutically acceptable salt or solvate thereof, is a compound of formula (Id-iia-v):

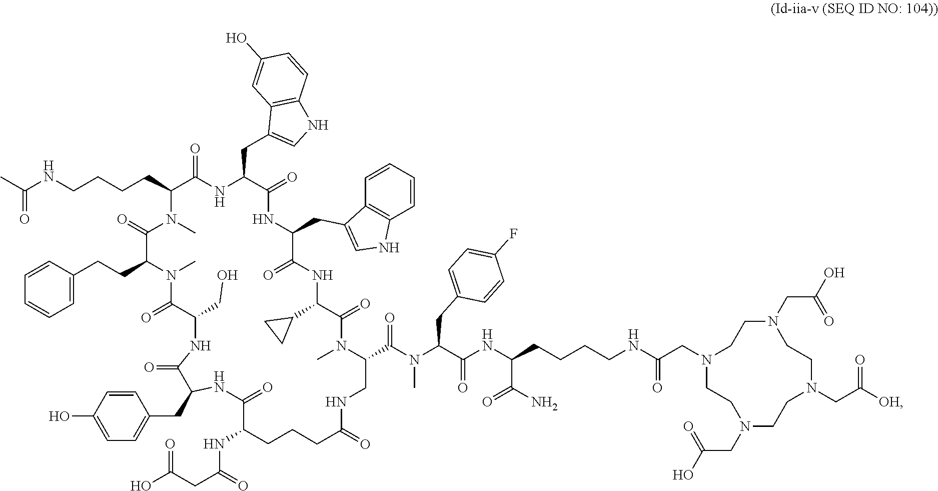 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(=O)CC(=O)O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-v) is radiolabeled with a radionuclide. In a further embodiment, the radionuclide is ⁶⁸Ga, ¹¹¹In, ¹⁷⁷Lu, or ²²⁵Ac.

In yet another embodiment of formula (Id-iia), or a pharmaceutically acceptable salt or solvate thereof, is a compound of formula (Id-iia-vi):

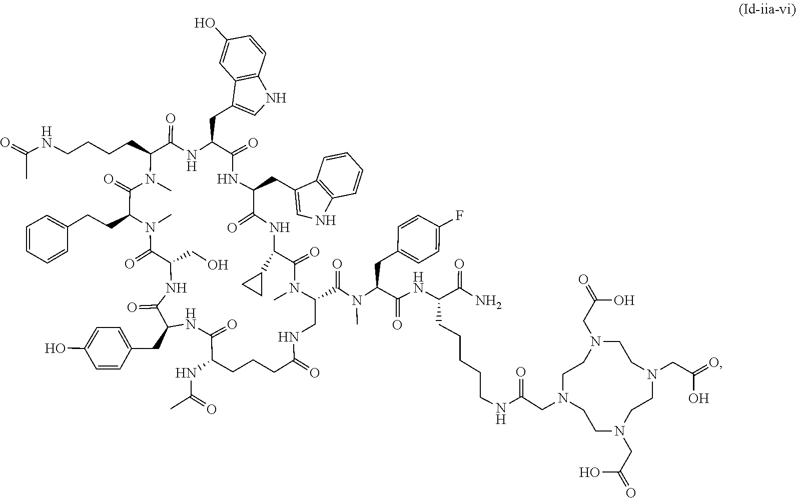 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-vi) is radiolabeled with a radionuclide. In a further embodiment, the radionuclide is ⁶⁸Ga, ¹¹¹In, ¹⁷⁷Lu, or ²²⁵Ac.

In still another embodiment of formula (Id-iia), or a pharmaceutically acceptable salt or solvate thereof, is a compound of formula (Id-iia-vii):

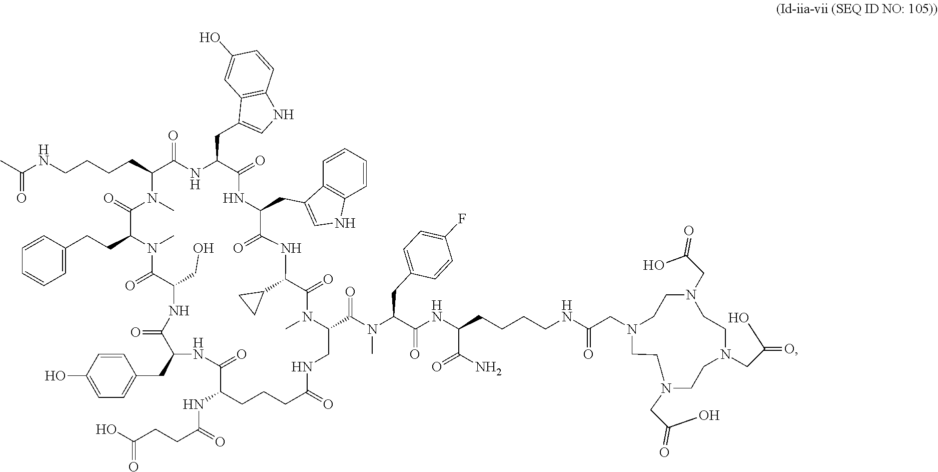 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(=O)CCC(=O)O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-vii) is radiolabeled with a radionuclide. In an embodiment, the radionuclide is ⁶⁸Ga, ¹¹¹In, ¹⁷⁷Lu, or ²²⁵Ac.

In another embodiment of formula (Id-iia), or a pharmaceutically acceptable salt or solvate thereof, is a compound of formula (Id-iia-viii):

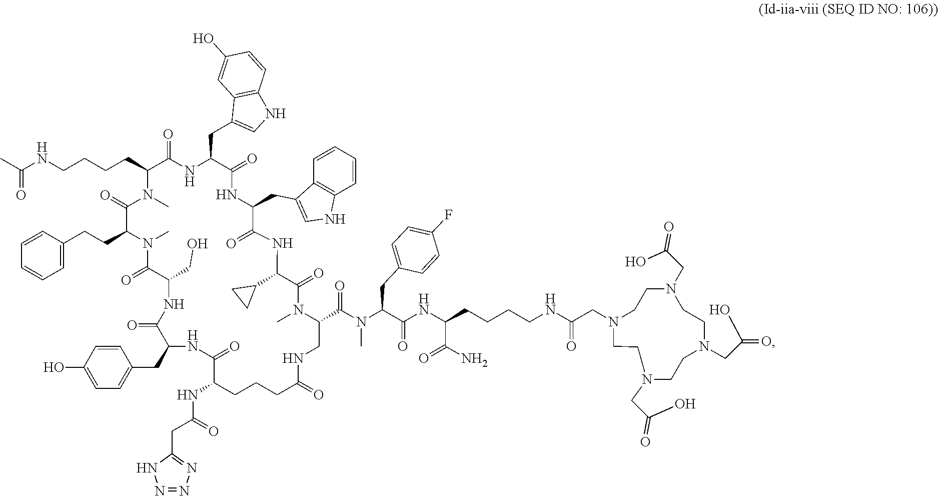 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(=O)Cc2nnn[nH]2)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-viii) is radiolabeled with a radionuclide. In a further embodiment, the radionuclide is ⁶⁸Ga, ¹¹¹In, ¹⁷⁷Lu, or ²²⁵Ac.

In yet another embodiment of formula (Id-iia), or a pharmaceutically acceptable salt or solvate thereof, is a compound of formula (Id-iia-ix):

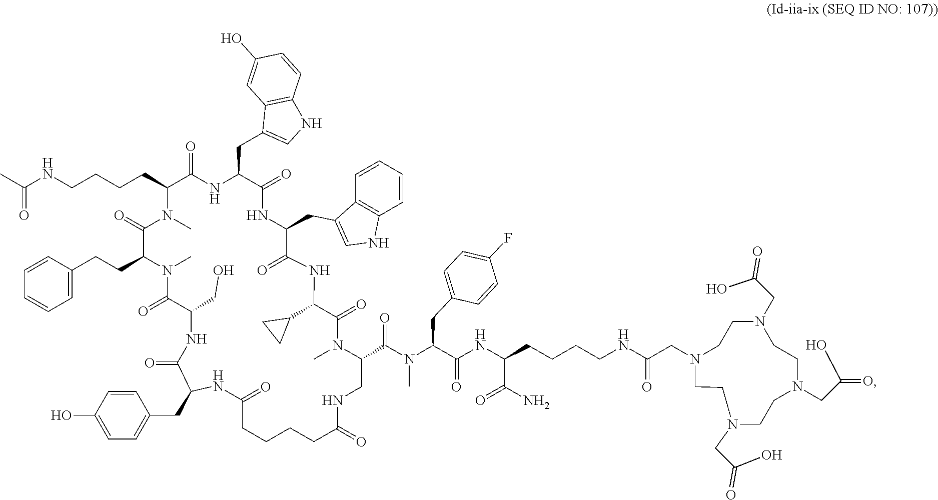 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCCCC(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-ix) is radiolabeled with a radionuclide. In a further embodiment, the radionuclide is ⁶⁸Ga, ¹¹¹In, ¹⁷⁷Lu, or ²²⁵Ac.

In yet another embodiment of formula (Id-iia), or a pharmaceutically acceptable salt or solvate thereof, is a compound of formula (Id-iia-x):

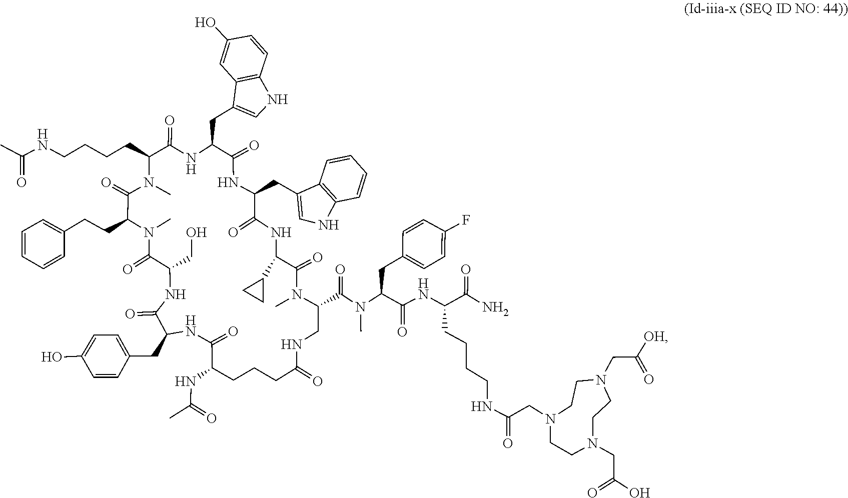 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iia-x) is radiolabeled with a radionuclide. In a further embodiment, the radionuclide is ⁶⁸Ga, ¹¹¹In, ¹⁷⁷Lu, or ²²⁵Ac.

In an embodiment of formula (Id-iib), or a pharmaceutically acceptable salt or solvate thereof, is a compound of formula (Id-iib-i):

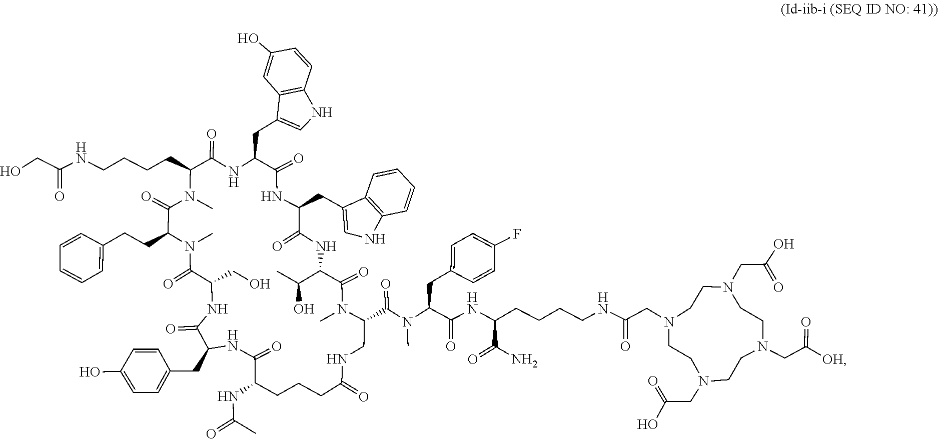 [SMILES: CC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H]([C@H](C)O)NC(=O)[C@H](Cc2c[nH]c3ccccc23)NC(=O)[C@H](Cc2c[nH]c3ccc(O)cc23)NC(=O)[C@H](CCCCNC(=O)CO)N(C)C(=O)[C@H](CCc2ccccc2)N(C)C(=O)[C@H](CO)NC(=O)[C@H](Cc2ccc(O)cc2)NC1=O]

which is optionally radiolabeled with a radionuclide defined elsewhere herein. In an embodiment, the compound of formula (Id-iib-i) is radiolabeled with a radionuclide. In a further embodiment, the radionuclide is ⁶⁸Ga, ¹¹¹In, ¹⁷⁷Lu, or ²²⁵Ac.

In another embodiment of formula (Id-iib), or a pharmaceutically acceptable salt or solvate thereof, is a compound of formula (Id-iib-ii):

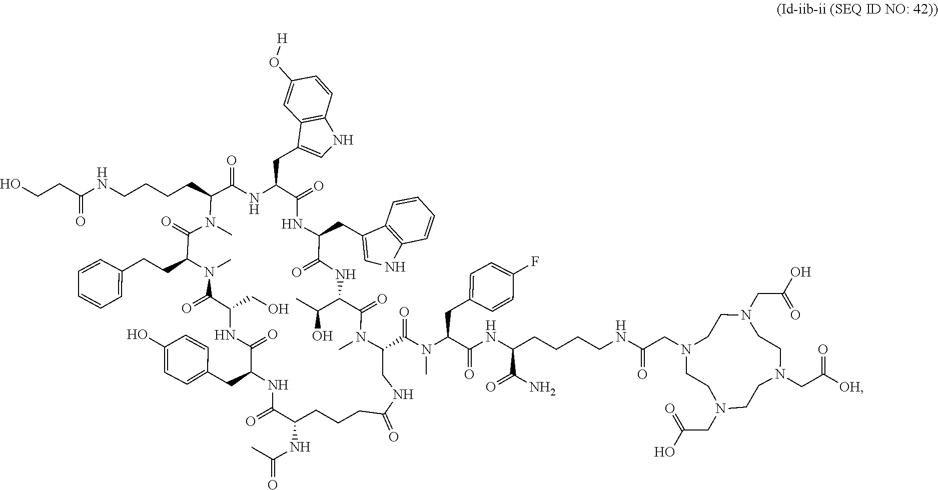

which is optionally radiolabeled with a radionuclide defined elsewhere herein. In an embodiment, the compound of formula (Id-iib-ii) is radiolabeled with a radionuclide. In a further embodiment, the radionuclide is ⁶⁸Ga, ¹¹¹In, ¹⁷⁷Lu, or ²²⁵Ac.

In an embodiment of formula (Id-iib), or a pharmaceutically acceptable salt or solvate thereof, is a compound of formula (Id-iib-iii):

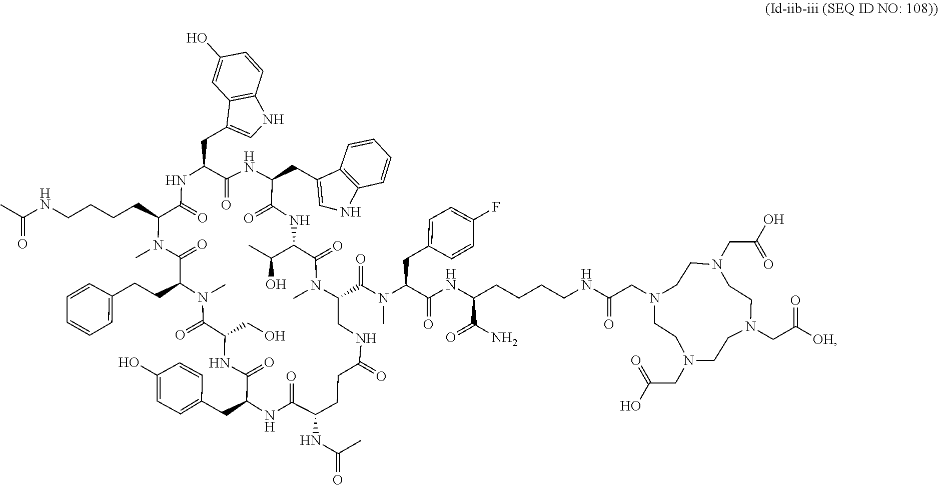 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H]([C@H](C)O)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iib-iii) is radiolabeled with a radionuclide. In a further embodiment, the radionuclide is ⁶⁸Ga, ¹¹¹In, ¹⁷⁷Lu, or ²²⁵Ac.

In another embodiment of formula (Id-iib), or a pharmaceutically acceptable salt or solvate thereof, is a compound of formula (Id-iib-iv):

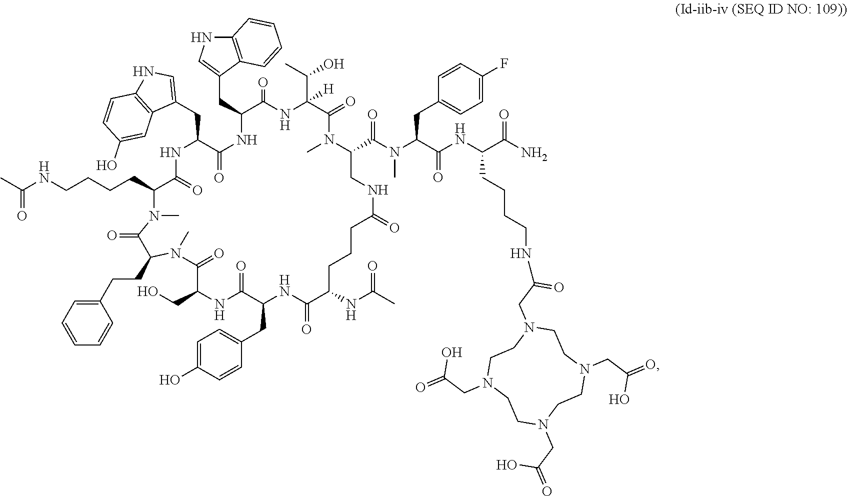 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H]([C@H](C)O)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

which is optionally radiolabeled with a radionuclide defined elsewhere herein. In an embodiment, the compound of formula (Id-iib-iv) is radiolabeled with a radionuclide. In a further embodiment, the radionuclide is ⁶⁸Ga, ¹¹¹In, ¹⁷⁷Lu, or ²²⁵Ac.

In an embodiment of formula (Id-iic), or a pharmaceutically acceptable salt or solvate thereof, is a compound of formula (Id-iic-i):

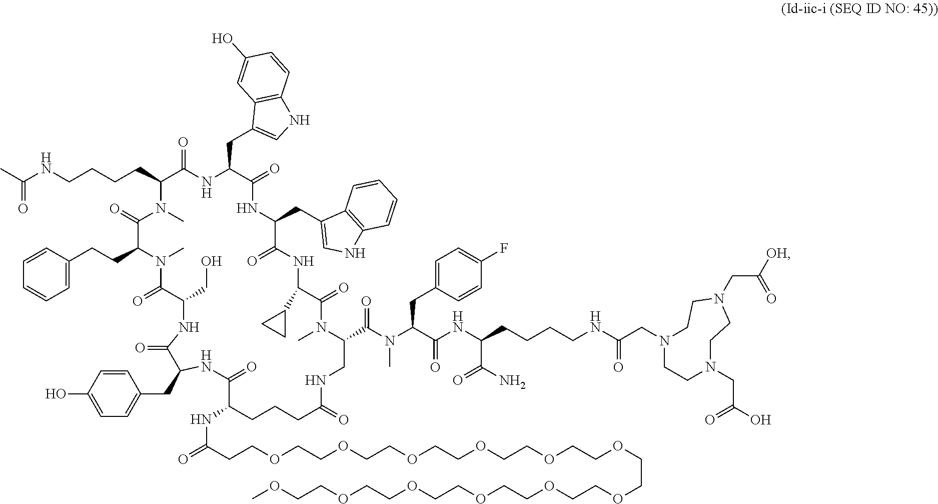 [SMILES: COCCOCCOCCOCCOCCOCCOCCOCCOCCOCCOCCOCCC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CC2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(C)=O)N(C)C(=O)[C@H](CCc2ccccc2)N(C)C(=O)[C@H](CO)NC(=O)[C@H](Cc2ccc(O)cc2)NC1=O]

which is optionally radiolabeled with a radionuclide defined elsewhere herein. In an embodiment, the compound of formula (Id-iic-i) is radiolabeled with a radionuclide. In a further embodiment, the radionuclide is ⁶⁸Ga, ¹¹¹In, ¹⁷⁷Lu, or ²²⁵Ac.

In another embodiment of formula (Id-iic), or a pharmaceutically acceptable salt or solvate thereof, is a compound of formula (Id-iic-ii):

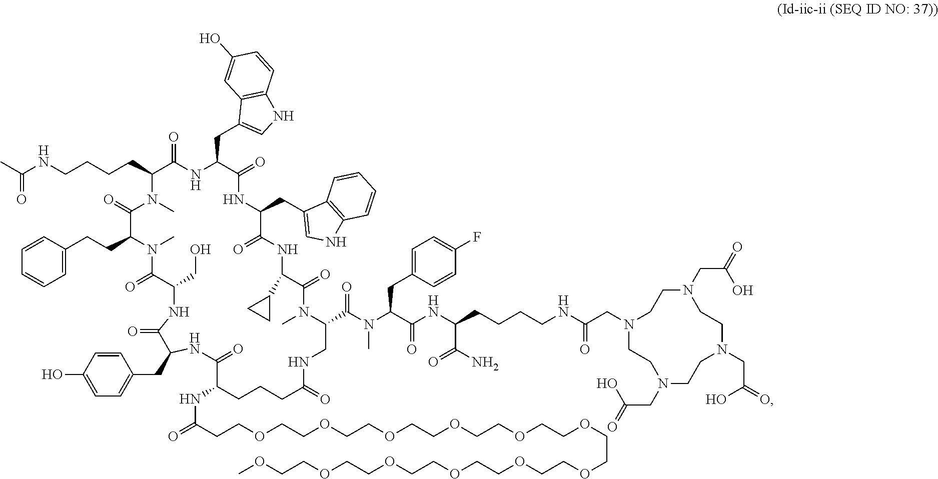 [SMILES: COCCOCCOCCOCCOCCOCCOCCOCCOCCOCCOCCOCCC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(C)=O)N(C)C(=O)[C@H](CCc2ccccc2)N(C)C(=O)[C@H](CO)NC(=O)[C@H](Cc2ccc(O)cc2)NC1=O]

which is optionally radiolabeled with a radionuclide defined elsewhere herein. In an embodiment, the compound of formula (Id-iic-ii) is radiolabeled with a radionuclide. In a further embodiment, the radionuclide is ⁶⁸Ga, ¹¹¹In, ¹⁷⁷Lu, or ²²⁵Ac.

Compounds Comprising PEG Chain and/or DOTA Chelator

In some embodiments, the compound of formula (Id) (and subgenera of formula (Id), e.g., formulae (Id-i) and (Id-ii)), or a pharmaceutically acceptable salt or solvate thereof, is 5 optionally substituted with one PEG chain.

In some embodiments, the compound of formula (Id) (and subgenera of formula (Id), e.g., formulae (Id-i) and (Id-ii)), or a pharmaceutically acceptable salt or solvate thereof, is substituted with one PEG chain.

In some embodiments, the compound of formula (Id) (and subgenera of formula (Id), e.g., formulae (Id-i) and (Id-ii)), or a pharmaceutically acceptable salt or solvate thereof, the PEG chain is bound to A¹.

In some embodiments, the compound of formula (Id) (and subgenera of formula (Id), e.g., formulae (Id-i) and (Id-ii)), or a pharmaceutically acceptable salt or solvate thereof, has the following definitions:
A¹ is substituted with a PEG chain, wherein A¹ and the PEG chain, together, have the structure of formula (A1-P):

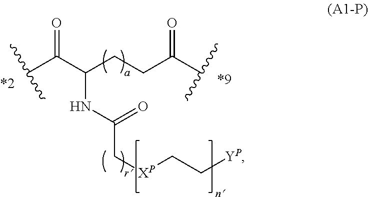

wherein:
X^{P} is selected from CH₂, N(R^{P}), and O;
Y^{P} is selected from H, —OH, —OC₁₋₆-alkyl, and —N(R^{P}) 2;
each R^{P} is independently selected from H and C₁₋₆-alkyl;
a is 1, 2, or 3;
n′ is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30; and
r′ is 0, 1, 2, 3, or 4;
A² is a moiety of formula (A2-I):

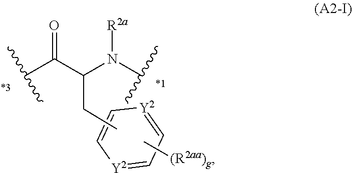

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2;
A³ is a moiety of formula (A3-II):

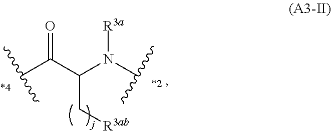

wherein:
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl; and
j is 1, 2, 3, 4, 5, or 6;
A⁴ is a moiety of formula (A4-I):

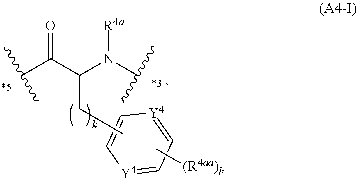

wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
k is 1, 2, 3, 4, 5, or 6; and
l is 0, 1, or 2;
A⁵ is a moiety of formula (A5-II):

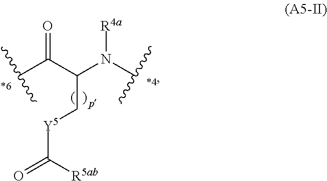

wherein:
Y⁵ is selected from O and NH;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
p′ is 1, 2, 3, 4, 5, or 6;
A⁶ is a moiety of formula (A6-Ia):

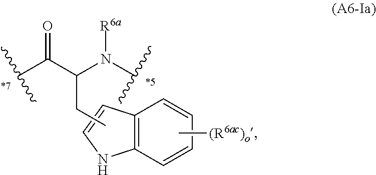

wherein:
R^{6a} is selected from H and C₁₋₃-alkyl;
R^{6ac} is selected from H, C₁₋₆-alkyl, and —C(O)—C₁₋₆-alkyl; and
o′ is 0, 1, or 2;
A⁷ is a moiety of formula (A7-Ia):

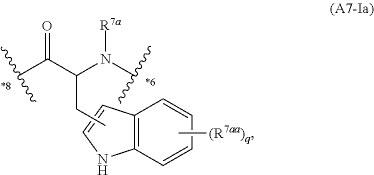

wherein:
R^{7a} is selected from H and C₁₋₃-alkyl;
each R^{7aa} is selected from halo, —NH₂, and —OH; and
q is 0, 1, or 2;
A⁸ is a moiety of formula (A8-I):

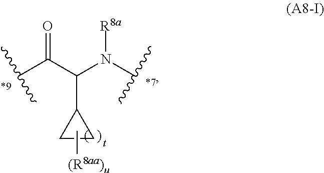

wherein:
R^{8a} is selected from H and C₁₋₃-alkyl;
R^{8aa} is selected from —CN, halo, —NH₂, and —OH;
t is 1 or 2; and
u is 0, 1, or 2;
A′ is a moiety of formula (A9-I):

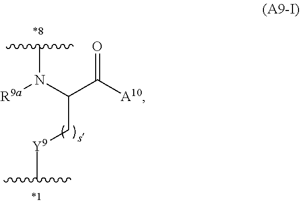

wherein:
Y′ is selected from C(O), NH, NC₁₋₆-alkyl, and S;
R^{9a} is selected from H and C₁₋₃-alkyl; and
s′ is 1 or 2; and
A¹⁰, L¹, and M, together, have the structure:

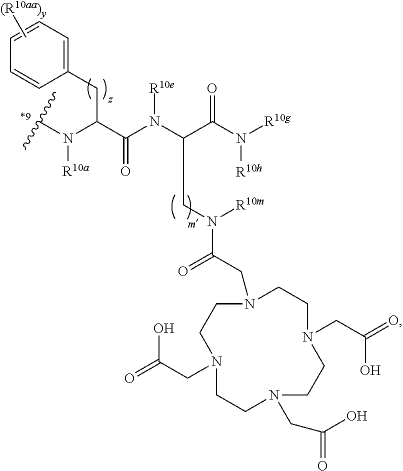

wherein:
R^{10a}, R^{10e}, and R^{10m} are each independently selected from H and C₁₋₃-alkyl;
R^{10g} and R^{10h} are each independently selected from H and C₁₋₆-alkyl;
each R^{10aa} is independently selected from C₁₋₆-alkyl, halo, —NH₂, —N₃, —OH, and —OC₁₋₆-alkyl;
y is 0, 1, or 2;
z is 1, 2, 3, 4, 5, or 6; and
m′ is 1, 2, 3, 4, 5, or 6,
wherein M is optionally radiolabeled with a radionuclide.

In an embodiment of formula (Id) (and subgenera of formula (Id), e.g., formulae (Id-i), (Id-ii), and (Id-iic)), or a pharmaceutically acceptable salt or solvate thereof, X^{P} is O; Y^{P} is —OC₁₋₆-alkyl; and n′ is 1, 2, 3, 4, 5, 6, 7, 8, 9, 10, 11, 12, 13, 14, or 15.

In some embodiments, the compound of formula (Id) (and subgenera of formula (Id), e.g., formulae (Id-i) and (Id-ii)), or a pharmaceutically acceptable salt or solvate thereof, has the following definitions:
A¹ is a moiety of formula (A1-I):

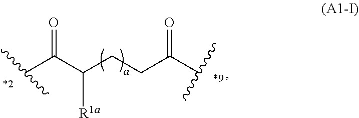

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl; and
a is 1, 2, or 3;
A² is a moiety of formula (A2-I):

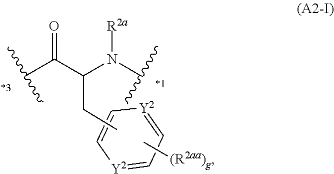

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2;
A³ is a moiety of formula (A3-II):

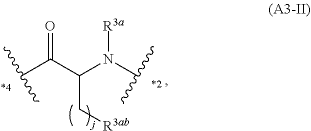

wherein:
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl; and
j is 1, 2, 3, 4, 5, or 6;
A⁴ is a moiety of formula (A4-I):

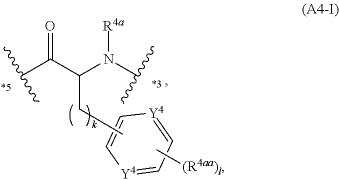

wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
k is 1, 2, 3, 4, 5, or 6; and
l is 0, 1, or 2;
A⁵ is a moiety of formula (A5-II):

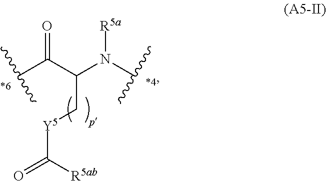

wherein:
Y⁵ is selected from O and NH;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
p′ is 1, 2, 3, 4, 5, or 6;
A⁶ is a moiety of formula (A6-Ia):

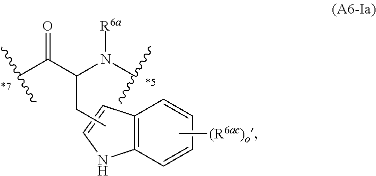

wherein:
R^{6a} is selected from H and C₁₋₃-alkyl;
R^{6ac} is selected from OH, C₁₋₆-alkyl, and —C(O)—C₁₋₆-alkyl; and
o′ is 0, 1, or 2;
A⁷ is a moiety of formula (A7-Ia):

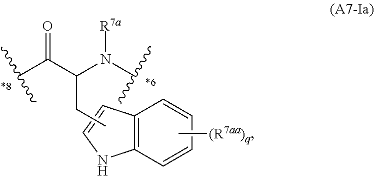

wherein:
R^{7a} is selected from H and C₁₋₃-alkyl;
each R^{7aa} is selected from —CN, halo, —NH₂, and —OH; and
q is 0, 1, or 2;
A⁸ is a moiety of formula (A8-I):

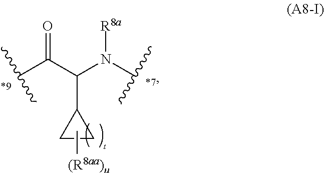

wherein:
R^{8a} is selected from H and C₁₋₃-alkyl;
R^{8aa} is selected from —CN, halo, —NH₂, and —OH;
t is 1 or 2; and
u is 0, 1, or 2;
A′ is a moiety of formula (A9-I):

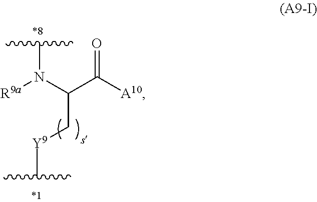

wherein:
Y⁹ is selected from C(O), NH, NC₁₋₆-alkyl, and S;
R^{9a} is selected from H and C₁₋₃-alkyl; and
s′ is 1 or 2; and
A¹⁰, L¹, and M, together, have the structure:

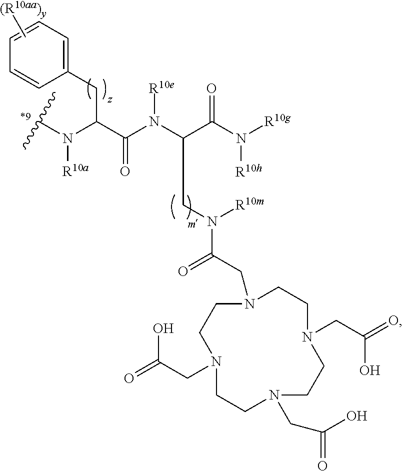

wherein:
R^{10a}, R^{10e}, and R^{10m} are each independently selected from H and C₁₋₃-alkyl;
R^{10g} and R^{10h} are each independently selected from H and C₁₋₆-alkyl;
each R^{10aa} is independently selected from C₁₋₆-alkyl, halo, —NH₂, —N₃, —OH, and —OC₁-6-alkyl;
y is 0, 1, or 2;
z is 1, 2, 3, 4, 5, or 6; and
m′ is 1, 2, 3, 4, 5, or 6,
wherein M is optionally radiolabeled with a radionuclide.

In some embodiments of the compound of formula (Id) (and subgenera of formula (Id), e.g., formulae (Id-i) and (Id-ii)), or a pharmaceutically acceptable salt or solvate thereof:

A², A³, and A⁷ are each in the L configuration.

In some embodiments, the compound of formula (Id) (and subgenera of formula (Id), e.g., formulae (Id-i) and (Id-ii)), or a pharmaceutically acceptable salt or solvate thereof, has the following definitions:
A¹ is a moiety of formula (A1-I):

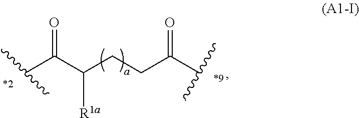

wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl; and
a is 1, 2, or 3;
A² is a moiety of formula (A2-Ia):

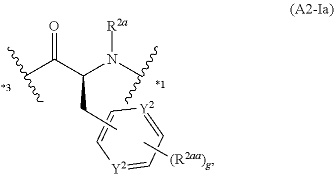

wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2;
A³ is a moiety of formula (A3-IIa):

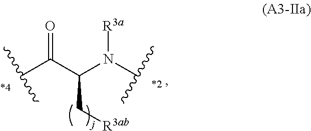

wherein:
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl; and
j is 1, 2, 3, 4, 5, or 6;
A⁴ is a moiety of formula (A4-I):

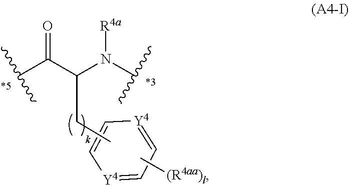

wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
k is 1, 2, 3, 4, 5, or 6; and
l is 0, 1, or 2;
A⁵ is a moiety of formula (A5-II):

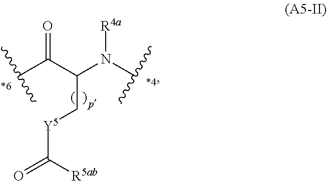

wherein:
Y⁵ is selected from O and NH;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
p′ is 1, 2, 3, 4, 5, or 6;
A⁶ is a moiety of formula (A6-Ia):

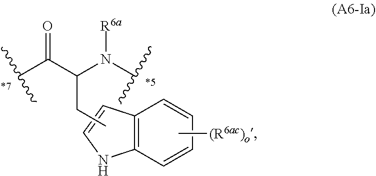

wherein:
R^{6a} is selected from H and C₁₋₃-alkyl;
R^{6ac} is selected from OH, C₁₋₆-alkyl, and —C(O)—C₁₋₆-alkyl; and
o′ is 0, 1, or 2;
A⁷ is a moiety of formula (A7-Iaaa):

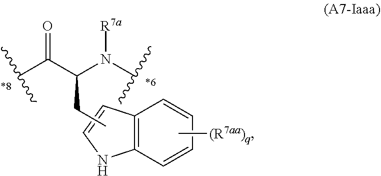

wherein:
R^{7a} is selected from H and C₁₋₃-alkyl;
each R^{7a} is selected from —CN, halo, —NH₂, and —OH; and
q is 0, 1, or 2;
A⁸ is a moiety of formula (A8-I):

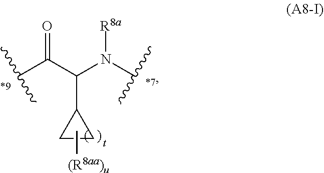

wherein:
R^{8a} is selected from H and C₁₋₃-alkyl;
R^{8aa} is selected from —CN, halo, —NH₂, and —OH;
t is 1 or 2; and
u is 0, 1, or 2;
A⁹ is a moiety of formula (A9-I):

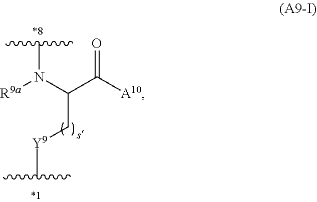

wherein:
Y⁹ is selected from C(O), NH, NC₁₋₆-alkyl, and S;
R^{9a} is selected from H and C₁₋₃-alkyl; and
s′ is 1 or 2; and
A¹⁰, L¹, and M, together, have the structure:

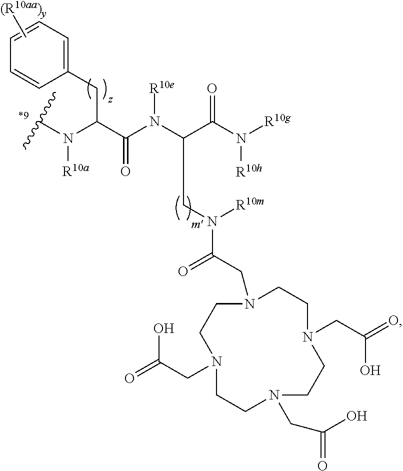

wherein:
R^{10a}, R^{10e}, and R^{10m} are each independently selected from H and C₁₋₃-alkyl;
R^{10g} and R^{10h} are each independently selected from H and C₁₋₆-alkyl;
each R^{10aa} is independently selected from C₁₋₆-alkyl, halo, —NH₂, —N₃, —OH, and —OC₁-6-alkyl;
y is 0, 1, or 2;
z is 1, 2, 3, 4, 5, or 6; and
m′ is 1, 2, 3, 4, 5, or 6,
wherein M is optionally radiolabeled with a radionuclide.

In an embodiment of formula (Id) (and subgenera of formula (Id), e.g., formulae (Id-i) and (Id-ii)), or a pharmaceutically acceptable salt or solvate thereof, at least one Y² is CH.

In another embodiment of formula (Id) (and subgenera of formula (Id), e.g., formulae (Id-i) and (Id-ii)), or a pharmaceutically acceptable salt or solvate thereof, each R^{2aa} is independently selected from halo, —OH, and —NH₂.

In yet another embodiment of formula (Id) (and subgenera of formula (Id), e.g., formulae (Id-i) and (Id-ii)), or a pharmaceutically acceptable salt or solvate thereof, R^{3ab} is selected from H, —OH, —C(O)OH, —C(O)NH₂, and —NH₂.

In still another embodiment of formula (Id) (and subgenera of formula (Id), e.g., formulae (Id-i) and (Id-ii)), or a pharmaceutically acceptable salt or solvate thereof, at least one Y⁴ is CH and wherein 1 is 0 or 1.

In an embodiment of formula (Id) (and subgenera of formula (Id), e.g., formulae (Id-i) and (Id-ii)), or a pharmaceutically acceptable salt or solvate thereof, R^{5b} is selected from —C₁₋₆-alkyl-O—C(O)R^{5c} and —C₁₋₆-alkyl-NH—C(O)R^{5c} and wherein R^{5c} is selected from C₁₋₆-alkyl and —OC₁₋₆-alkyl.

In another embodiment of formula (Id) (and subgenera of formula (Id), e.g., formulae (Id-i) and (Id-ii)), or a pharmaceutically acceptable salt or solvate thereof, o′ is 0 or 1.

In yet another embodiment of formula (Id) (and subgenera of formula (Id), e.g., formulae (Id-i) and (Id-ii)), or a pharmaceutically acceptable salt or solvate thereof, q is 0 or 1.

In still another embodiment of formula (Id) (and subgenera of formula (Id), e.g., formulae (Id-i) and (Id-ii)), or a pharmaceutically acceptable salt or solvate thereof, t is 1 and wherein u is 0 or 1.

In an embodiment of formula (Id) (and subgenera of formula (Id), e.g., formulae (Id-i) and (Id-ii)), or a pharmaceutically acceptable salt or solvate thereof, Y⁹ is selected from NH and NC₁₋₆-alkyl.

In another embodiment of formula (Id) (and subgenera of formula (Id), e.g., formulae (Id-i) and (Id-ii)), or a pharmaceutically acceptable salt or solvate thereof, each R^{10aa} is independently selected from halo, —NH₂, —N₃, and —OH.

In yet another embodiment of formula (Id) (and subgenera of formula (Id), e.g., formulae (Id-i) and (Id-ii)), or a pharmaceutically acceptable salt or solvate thereof, the cyclic peptide, P, is selected from Examples A1-A74 and E1-E3.

In yet another embodiment of formula (Id) (and subgenera of formula (Id), e.g., formulae (Id-i) and (Id-ii)), or a pharmaceutically acceptable salt or solvate thereof, the cyclic peptide, P, is selected from Examples A75-A77, E4, and E5.

In certain embodiments, the compound of formula (Id), or a pharmaceutically acceptable salt or solvate thereof, is a compound selected from Examples B1-B7, C1-C₃, C5-C30, and D1-D5, F1-F4, G1, H1-H4, I1, and I2.

In certain embodiments, the compound of formula (Id), or a pharmaceutically acceptable salt or solvate thereof, is a compound selected from Examples B9-B13, C31-C37, D6, D7, J1, and J2.

In some embodiments, the compound of formula (Ib) is a compound of formula (ib-ii):

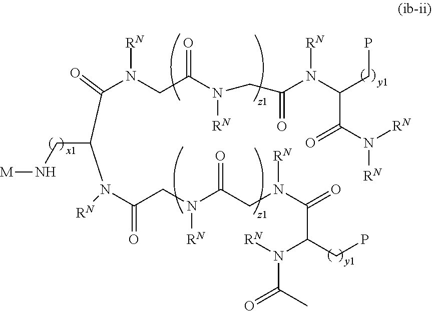

or a pharmaceutically acceptable salt or solvate thereof,
   wherein:
R^{N} is H or C₁₋₆-alkyl;
x1 and y1, independently, at each occurrence, are 0, 1, 2, 3, 4, 5, or 6;
z1, independently at each occurrence, is 0, 1, 2, 3, 4, 5, 6, 7, 8, 9, 10, 11, 12, 13, 14, 15, 16, 17, 18, 19, or 20;
each P is independently a cyclic (HER2-targeting) peptide; and
M is a chelating agent optionally radiolabeled with a radionuclide; and wherein the cyclic peptide, chelating agent, and radionuclide are as disclosed elsewhere herein. In an embodiment, M is radiolabeled with ⁶⁸Ga or ¹⁷⁷Lu. In another embodiment, M is radiolabeled with ¹¹¹In or ²²⁵Ac.

In some embodiments of the compound of formulae (Ib), (Ib-i), and (Ib-ii), or a pharmaceutically acceptable salt or solvate thereof, is a compound selected from Examples B8 and C4.

### Compounds Comprising Albumin Binders

The compound of formula (Id), or a pharmaceutically acceptable salt or solvate thereof, may further comprise an albumin binding moiety attached to any of P, L¹ or M. The albumin binder may be as disclosed elsewhere herein. In some embodiments, an albumin binding moiety is attached to the compound of formula (Id), or a pharmaceutically acceptable salt or solvate thereof, via a side chain of any of A¹-A¹⁰ of the cyclic peptide. In some embodiments, an albumin binding moiety is attached to the compound of formula (Id), or a pharmaceutically acceptable salt or solvate thereof, via a functional group on L¹. In some embodiments, an albumin binding moiety is attached to the compound of formula (Id), or a pharmaceutically acceptable salt or solvate thereof, via a functional group on M. For example, the compound of formula (Id), may be a compound of formula (Id-iii), (Id-iv), or (Id-v):

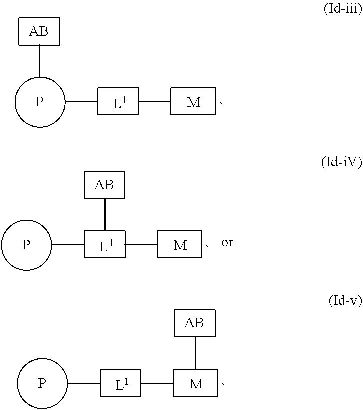

wherein AB is an albumin binder as disclosed elsewhere herein. In some embodiments of formulae (Id-iii), (Id-iv), and (Id-v), or a pharmaceutically acceptable salt or solvate thereof, M is a chelator that is not radiolabeled with a radionuclide. In embodiments of formulae (Id-iii), (Id-iv), and (Id-v), or a pharmaceutically acceptable salt or solvate thereof, M is a chelator that is radiolabeled with a radionuclide.

In various embodiments, the compound of formula (Id), or a pharmaceutically acceptable salt or solvate thereof, is a compound of formula (Id-iii), or a pharmaceutically acceptable salt or solvate thereof. In certain embodiments, the compound of formula (Id-iii), or a pharmaceutically acceptable salt or solvate thereof, is a compound that is Example G1.

In certain embodiments, the HER2-targeting compound is a compound of formula (I):

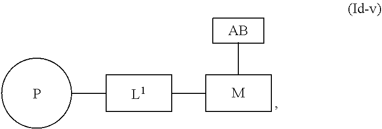

or a pharmaceutically acceptable salt or solvate thereof,
where n is 2, and each L¹ is the same or different and each M is different. For example, in some embodiments, the compound of (I), or a pharmaceutically acceptable salt or solvate thereof, is a compound of formula (Ie):

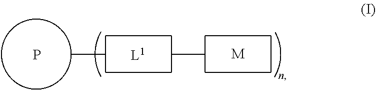

or a pharmaceutically acceptable salt or solvate thereof,
   wherein:
P is a cyclic HER2-targeting peptide;
M¹ is an imaging agent;
M^{C} is a chelator, optionally radiolabeled with a radionuclide;
L^{1I} is a bond or linker, connecting the imaging agent to P; and
L^{1C} is a bond or linker connecting the chelator to P;
wherein L^{1I} and L^{1C} are the same or different, and wherein the cyclic HER2-targeting peptide, imaging agent, chelator, radionuclide, and linker are as disclosed elsewhere herein.

In embodiments of the compound of formula (Ie), or a pharmaceutically acceptable salt or solvate thereof, M¹ is an imaging agent as previously described and M^{C} is a chelator radiolabeled with a radionuclide. In some embodiments of the compound of formula (Ie), or a pharmaceutically acceptable salt or solvate thereof, M^{C} is a chelator radiolabeled with a therapeutically active radionuclide. In some embodiments of the compound of formula (Ie), or a pharmaceutically acceptable salt or solvate thereof, M^{C} is a chelator radiolabeled with a diagnostically active radionuclide. In further embodiments of the compound of formula (Ie), or a pharmaceutically acceptable salt or solvate thereof, the chelator is DOTA, and the radionuclide is ¹⁷⁷Lu or ⁶⁸Ga. In still further embodiments of the compound of formula (Ie), or a pharmaceutically acceptable salt or solvate thereof, the imaging agent is Sulfo Cy5.

In certain embodiments, the compound of formula (Ie), or a pharmaceutically acceptable salt or solvate thereof, is a compound that is Example D6.

The compound of formula (Ie), or a pharmaceutically acceptable salt or solvate thereof, may further comprise an albumin binder as described above. In some embodiments, when the compound of formula (Ie), or a pharmaceutically acceptable salt or solvate thereof, comprises an albumin binder, the albumin binder is attached to the compound via a side chain of any one of A¹-A¹⁰ of the cyclic peptide. In some embodiments, when the compound of formula (Ie), or a pharmaceutically acceptable salt or solvate thereof, comprises an albumin binder, the albumin binder is attached to the compound via a functional group on L^{1I}. In some embodiments, when the compound of formula (Ie), or a pharmaceutically acceptable salt or solvate thereof, comprises an albumin binder, the albumin binder is attached to the compound via a functional group on L^{1C}. In some embodiments, when the compound of formula (Ie), or a pharmaceutically acceptable salt or solvate thereof, comprises an albumin binder, the albumin binder is attached to the compound via a functional group on M¹. In some embodiments, when the compound of formula (Ie), or a pharmaceutically acceptable salt or solvate thereof, comprises an albumin binder, the albumin binder is attached to the compound via a functional group on M^{C}.

In some embodiments, the compound of formula (I), is a compound of formula (If):

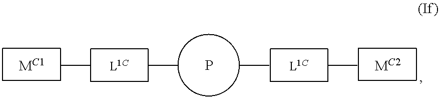

or a pharmaceutically acceptable salt or solvate thereof,
   wherein:
P is a cyclic HER2-targeting peptide;
M^{C1} is a chelator optionally radiolabeled with a radionuclide;
M^{C2} is a chelator optionally radiolabeled with a radionuclide; and
each L^{1C} is independently selected from a bond and a linker, and wherein the cyclic HER2-targeting peptide and each chelator, radionuclide, and linker is as previously described.

In some embodiments of the compound of Formula (If), or a pharmaceutically acceptable salt or solvate thereof, M^{C1} and M^{C2} are the same. In some embodiments of the compound of Formula (If), or a pharmaceutically acceptable salt or solvate thereof, M^{C1} and M^{C2} are different. In some embodiments of the compound of Formula (If), each L^{1C} is the same. In some embodiments of the compound of Formula (If), or a pharmaceutically acceptable salt or solvate thereof, each L^{1C} is different.

The compound of formula (If), or a pharmaceutically acceptable salt or solvate thereof, may further comprise an albumin binder as described elsewhere herein.

In some embodiments, the compound of formula (I), or a pharmaceutically acceptable salt or solvate thereof, is a compound of formula (Ig):

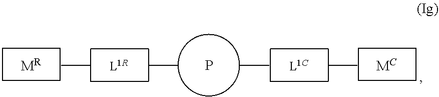

or a pharmaceutically acceptable salt or solvate thereof,
   wherein:
P is a cyclic HER2-targeting peptide;
M^{R} is a radionuclide;
M^{C} is a chelator optionally radiolabeled with a radionuclide;
L^{1R} is a bond or a linker; and
L^{1C} is a bond or a linker,
wherein the cyclic HER2-targeting peptide, radionuclide, chelator, and linker are as previously described.

In certain embodiments of formula (Ig), or a pharmaceutically acceptable salt or solvate thereof, M^{R} is a radiohalogen and L^{1R} is a bond. In some embodiments of formula (Ig), or a pharmaceutically acceptable salt or solvate thereof, when M^{R} is a radiohalogen and L^{1R} is a bond, then M^{C} is a chelator radiolabeled with a radionuclide other than a radiohalogen, and L^{1C} is a linker.

The compound of formula (Ig) may further comprise an albumin binder as described elsewhere herein.

In certain embodiments, the HER2-targeting compound is a compound of formula (Ib) or (Ic):

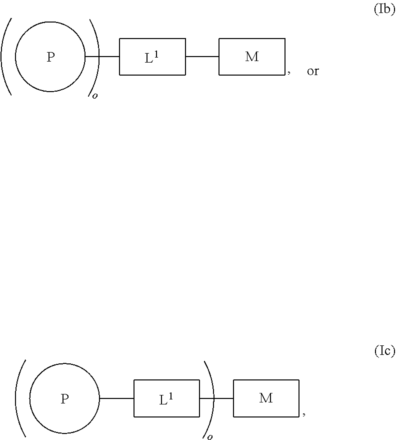

or a pharmaceutically acceptable salt or solvate thereof,
wherein o is 2, each L¹ is the same or different and each P is different. For example, in some embodiments, the compound of formula (Ib) is a compound of formula (Ih):

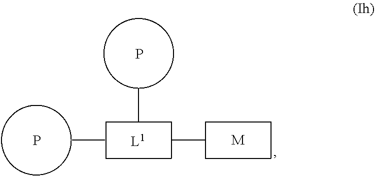

or a pharmaceutically acceptable salt or solvate thereof,
   wherein:
each P is independently a cyclic HER2-targeting peptide;
L¹ is a bond or a linker; and
M is an imaging agent, a chelator optionally radiolabeled with a radionuclide, or a radionuclide, and wherein the HER2-targeting cyclic peptides, linker, imaging agent, chelator, and radionuclide are as described elsewhere herein.

In some embodiments of the compound of formula (Ih), or a pharmaceutically acceptable salt or solvate thereof, each P is the same. In some embodiments of the compound of formula (Ih), or a pharmaceutically acceptable salt or solvate thereof, each P is different. Each HER2-targeting cyclic peptide of the compound of formula (Ih) may be different by one or more amino acids, two or more amino acids, three or more amino acids, four or more amino acids, five or more amino acids, six or more amino acids, seven or more amino acids, eight or more amino acids, or nine or more amino acids. In some embodiments, the HER2-targeting cyclic peptides of the compound of formula (Ih) are different by no more than one amino acid, no more than two amino acids, no more than three amino acids, or no more than four amino acids.

In various embodiments of the compound of formula (Ih), or a pharmaceutically acceptable salt or solvate thereof, the compound further comprises an albumin binder (as described elsewhere herein).

In some embodiments, the compound of formula (Ic) is a compound of formula (Ii):

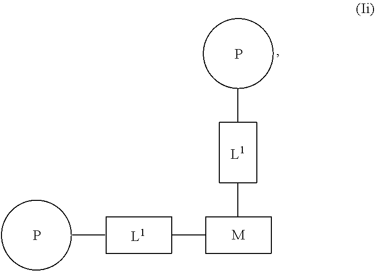

or a pharmaceutically acceptable salt or solvate thereof,
   wherein:
each P is independently a cyclic HER2-targeting peptide;
each L¹ is selected from a bond and a linker; and
M is an imaging agent, a chelator optionally radiolabeled with a radionuclide, or a radionuclide, and wherein the HER2-targeting cyclic peptides, linker, imaging agent, chelator, and radionuclide are as described elsewhere herein.

In some embodiments of the compound of formula (Ii), or a pharmaceutically acceptable salt or solvate thereof, each P is the same. In some embodiments of the compound of formula (Ii), or a pharmaceutically acceptable salt or solvate thereof, each P is different. In some embodiments of the compound of Formula (Ih), or a pharmaceutically acceptable salt or solvate thereof, each L¹ is the same. In some embodiments of the compound of Formula (Ii), or a pharmaceutically acceptable salt or solvate thereof, each L¹ is different.

Each HER2-targeting cyclic peptide of the compound of formula (Ii) may be different by one or more amino acids, two or more amino acids, three or more amino acids, four or more amino acids, five or more amino acids, six or more amino acids, seven or more amino acids, eight or more amino acids, or nine or more amino acids. In some embodiments, the HER2-targeting cyclic peptides of the compound of formula (Ii) are different by no more than one amino acid, no more than two amino acids, no more than three amino acids, or no more than four amino acids.

In various embodiments of the compound of formula (Ii), the compound further comprises an albumin binder. The albumin binder may be as described elsewhere herein.

In any of formulae (Id)-(Ii), and any other combination of P, L¹, and M encompassed by the present disclosure, the cyclic peptide may be further connected to various moieties that may increase or supplement the activity of the disclosed HER2-targeting compounds. Moieties that may be connected to any of the compounds described above include, but are not limited to, albumin binders, additional HER2-targeting agents, additional imaging agents, and cytotoxic drugs.

### Methods of Synthesis

The compounds described herein may be synthesized by many techniques that are known to those skilled in the art. In some aspects, the present disclosure provides a method of chemically synthesizing a radioligand peptide of the present disclosure. In some embodiments, a portion of the peptide is recombinantly synthesized, rather than chemically synthesized. In some embodiments, methods of producing a radioligand peptide include cyclizing the peptide portion after all the constituents have been attached. In other embodiments, methods of producing a radioligand peptide include cyclizing the peptide prior to attachment of all the constituents to one another. In particular embodiments, cyclization is accomplished via any of the various methods described herein.

In some embodiments, the peptide is first synthesized and then covalently attached to the linker. The chelator may be attached to the linker before or after attachment of the peptide. For example, in some embodiments, the peptide is attached to the linker to form a linker-peptide intermediate that is then attached to the chelator. In other embodiments, the chelator is attached to the linker to form a linker-chelator intermediate that is then attached to the peptide.

In some embodiments, one or more of the amino acid residues or amino acid are covalently attached to one another and then attached to the linker at an intermediate oligomer stage before attaching additional amino acids and cyclization to form a peptide of the disclosure. In such embodiments, the intermediate oligomer may comprise the linker and a fragment of the peptide sequence or the linker, the chelator, and a fragment of the peptide sequence. As will be appreciated by one of ordinary skill in the art, the fragment of the peptide sequence may have a sequence length any number of amino acids shorter than the length of the complete peptide sequence. For example, if the peptide sequence is 10 residues long, then a fragment of that sequence may be 1, 2, 3, 4, 5, 6, 7, 8, or 9 residues long. Illustrative synthetic methods are described in the Examples.

### Pharmaceutical Compositions and Dosing

The present disclosure further provides a pharmaceutical composition comprising HER2-targeting radioligand described herein. In particular, a pharmaceutical composition of the present disclosure includes one or more radioligand peptides disclosed herein and a pharmaceutically acceptable carrier, diluent, or excipient. The pharmaceutically acceptable carrier, diluent or excipient may be a solid, semi-solid or liquid filler, diluent, encapsulating material or formulation auxiliary of any type.

The pharmaceutical compositions may be administered parenterally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered by parenteral administration. In particular, a pharmaceutical composition of the present disclosure may be formulated for and administered by intravenous administration.

Radioligands of the present disclosure may be prepared and/or formulated as pharmaceutically acceptable salts and/or other forms thereof or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts can be found in Remington: The Science and Practice of Pharmacy, 21st Edition, Lippincott Wiliams and Wilkins, Philadelphia, Pa., 2006.

In certain aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain additives such as preservatives, wetting agents, emulsifying agents, chelating agents, buffering agents, and dispersing agents.

In some embodiments, the pharmaceutical composition comprises a chelating agent to sequester internally deposited radionuclides. Any of the chelating agents listed in Tables 3-7 above may be included in pharmaceutical compositions comprising the HER2-targeting radioligand. In various embodiments, the pharmaceutical composition comprises DTPA. Additional exemplary chelating agents that may be included in pharmaceutical compositions of the present disclosure are described in Holik, et al. “The Chemical Scaffold of Theranostic Radiopharmaceuticals: Radionuclide, Bifunctional Chelator, and Pharmacokinetics Modifying Linker.” Molecules 27.10 (2022): 3062 and Kostelnik, Thomas I., and Chris Orvig. “Radioactive main group and rare earth metals for imaging and therapy.” Chemical Reviews 119.2 (2018): 902-956, both of which are incorporated by reference in their entireties.

In some embodiments, the pharmaceutical composition comprises one or more buffering agents to maintain a pH of about 3 to 5. Suitable buffering agents include, but are not limited to acetate, citrate, Tris, lactate, and tartrate, and the acid forms thereof.

Pharmaceutical compositions including the HER2-targeting radioligand of the instant disclosure may further comprise a stabilizer, such as, for example, a free radical scavenger, in order to prevent autoradiolysis of the inventive radioligand. Suitable stabilizers for inclusion in the disclosed pharmaceutical compositions include, but are not limited to, 2,5-dihydroxybenzoic acid or salts thereof, ascorbic acid or salts thereof, gentisic acid or salts thereof, methionine, histidine, melatonine, N-acetylmethionine, ethanol, an amino acid infusion solution, or any combination thereof. In some embodiments, the pharmaceutical composition includes a gentisic acid stabilizer. In some embodiments, the pharmaceutical composition includes an ascorbic acid stabilizer. In some embodiments, the pharmaceutical composition include stabilizer including gentisic acid and ascorbic acid.

In some embodiments, injectable compositions including a HER2-targeting radioligand are administered by infusion. For example, a composition of the present disclosure may be administered to a subject by an infusion ranging from about 1 to about 120 minutes in duration. In some embodiments, a composition of the present disclosure may be administered to a subject by an infusion ranging from about 1 to about 60 minutes in duration. In some embodiments, a composition of the present disclosure may be administered to a subject by an infusion ranging from about 1 to about 30 minutes in duration. In some embodiments, a composition of the present disclosure may be administered to a subject by an infusion ranging from about 1 to about 20 minutes in duration. In some embodiments, a composition of the present disclosure may be administered to a subject by an infusion ranging from about 1 to about 10 minutes in duration. In some embodiments, a composition of the present disclosure may be administered to a subject by an infusion ranging from about 5 to about 10 minutes in duration.

In some embodiments, the pH of the disclosed pharmaceutical compositions ranges from about 3 to about 11. The pH of the compositions may, for example, range from about 3 to about 7 or from about 3 to about 5.

The total daily usage of the HER2-targeting ligands (e.g., HER2-targeting radioligands) and compositions of the present disclosure can be decided by the attending physician within the scope of reasonable medical judgment. The specific therapeutically effective dose level for any particular subject will depend upon a variety of factors including: a) the disorder being treated and the severity of the disorder; b) activity of the specific compound employed; c) the specific composition employed; d) the age, body weight, general health, sex and diet of the patient; e) the time route, and mechanism of administration; f) the rate of excretion of the specific compound employed; g) the duration of the treatment (including the number of cycles in a treatment); h) administration of combination partners (e.g., chemotherapies and/or imaging agents), and like factors well known in the medical arts.

Provided herein are radiopharmaceuticals or radioligands targeting HER2. In particular embodiments, the dosing of a HER2-targeting radioligands of the present disclosure to be administered to a human or other mammal host in single or divided doses may be referred to as administered activity to be delivered to the subject (e.g., human or other mammal host). Administered activity of radiopharmaceuticals is given in units of radioactive disintegrations per unit time (SI units Becquerels (BQ), imperial units Curies (Ci)). The total dose will vary depending on the purpose, i.e., for imaging and/or therapy, and the number of cycles of administration. Dosing may also be based on energy deposited per unit of mass, i.e., the absorbed dose, usually given in J/kg or Gy).

The dosing of the radio-labeled compounds will also be determined by the particular radionuclide, and whether said radionuclide is a β-emitter (can also be referred to in the art as a beta-minus emitter) (e.g., ⁹⁰Y, ¹³¹I, ¹⁵³Sm, or ¹⁷⁷Lu) or an α-emitter (e.g., ²¹¹At, ²¹²Pb, ²¹²Bi, ²²³Ra, ²²⁵Ac, or ²²⁷Th).

In some embodiments, the total dose (over the course of a treatment regimen) of the HER2-targeting ligand radiolabeled with a β-emitter such as, e.g., ¹⁷⁷Lu, is from about 1 GBq to about 200 GBq. In some embodiments, the HER2-targeting radioligand comprising a β-emitter is administered in a total dose to deliver from 10 to 60 GBq of radiation. In some embodiments, the HER2-targeting radioligand comprising a β-emitter is administered in a total dose to deliver from 20 to 80 GBq of radiation. In some embodiments, the HER2-targeting radioligand comprising a β-emitter is administered in a total dose to deliver from 40 to 100 GBq of radiation. In some embodiments, the HER2-targeting radioligand comprising the β-emitter is administered in a single dose (once within a 24-hour period) to deliver from about 1 to about 20 GBq of radiation. In some embodiments, the HER2-targeting radioligand comprising the β-emitter is administered in a single dose (once within a 24-hour period) to deliver from about 1 to about 15 GBq of radiation. In some embodiments, the HER2-targeting radioligand comprising the β-emitter is administered in a single dose (once within a 24-hour period) to deliver from about 1 to about 10 GBq of radiation.

In some embodiments, the total dose (over the course of a treatment regimen) of the HER2-targeting ligand radiolabeled with an α-emitter, e.g., ²²⁵Ac, is from about 1 MBq to about 100 MBq. In some embodiments, the HER2-targeting radioligand comprising an α-emitter is administered in a total dose of from about 10 to about 60 MBq of radiation. In some embodiments, the HER2-targeting radioligand comprising an α-emitter is administered in a total dose of from about 20 to about 80 MBq of radiation. In some embodiments, the HER2-targeting radioligand comprising the α-emitter is administered in a single dose (once within a 24-hour period) to deliver from about 1 to about 40 MBq of radiation. In some embodiments, the HER2-targeting radioligand comprising the α-emitter is administered in a single dose (once within a 24-hour period) to deliver from about 5 to about 40 MBq of radiation. In some embodiments, the HER2-targeting radioligand comprising the α-emitter is administered in a single dose (once within a 24-hour period) to deliver from about 5 to about 25 MBq of radiation.

### Combination Therapies

Also provided herein are combinations (e.g., combination therapies) comprising at least one HER2-targeting ligand of the present disclosure and one or more other therapeutically active agents. The disclosed pharmaceutical combinations may be used in the treatment or prevention of HER2-related diseases, such as, for example, cancer. The HER2-targeting ligand of the present disclosure, when radiolabeled with a suitable imaging and/or therapeutic radionuclide, may be used in the treatment of a cancer wherein the subject is also receiving standard of care treatment or other chemotherapies approved for the treatment of the cancer. Suitable pharmaceutical combinations may include a HER2-targeting compound described herein and one or more immune checkpoint inhibitors, such as, for example, PD-1 and PD-L¹ inhibitors, CTLA-4 inhibitors, and LAG-3 inhibitors; selected estrogen receptor modulators; cyclin-dependent kinase (CDK) inhibitors, such as, e.g., CDK4 and CDK6 inhibitors; steroidal and nonsteroidal aromatase inhibitors; hormone receptor antagonists and inhibitors; antineoplastic agents; antimitotic agents; mTOR kinase inhibitors; antimetabolites; antifolates; DNA intercalators; tyrosine kinase inhibitors; and/or nonsteroidal anti-inflammatory drugs (NSAIDs).

### Methods of Treatment

The present disclosure also provides a method of treating one or more HER2-related diseases or disorders in a subject in need thereof, the method comprising administering a therapeutically effective amount of a HER2 targeting ligand/therapeutic or radioligand therapeutic described herein to the subject. The HER2 targeting ligands or compounds of the present disclosure may be administered to a subject having any HER2-related disease or disorder, including, but not limited to: proliferation diseases, such as, for example, cancer. In certain embodiments, the HER2-related disease or disorder is cancer.

In an aspect, provided herein is a method treating cancer in a subject in need thereof comprising administering a therapeutically effective amount of, e.g., a compound of any of Formulae (I), (Ia), (Ib), (Ic), (Id) and (I-i) particularly a radiolabeled compound of any of Formulae (I), (Ia), (Ib), (Ic), (Id) and (I-i). In an embodiment, the cancer expresses HER2.

In some aspects, the HER2-targeting therapeutic (e.g., a HER2-targeting radioligand therapeutic), or pharmaceutical composition thereof, is used as a curative or adjuvant cancer treatment. In other aspects, the HER2-targeting therapeutic (e.g., a HER2-targeting radioligand therapeutic), or pharmaceutical composition thereof, is used as a palliative treatment. Methods of palliative treatment using the disclosed radioligands include local disease control and/or symptomatic relief.

The HER2-targeting therapeutics or ligands (e.g., a HER2-targeting radioligand therapeutic) described herein may be used as the primary therapy for treatment of HER2-related disease or disorders, such as cancer. Alternatively, the HER2-targeting therapeutics or ligands (e.g., a HER2-targeting radioligand therapeutic) of the instant disclosure may be the secondary, tertiary, or final therapy for a HER2-related disease or disorder.

The HER2-targeting compounds of the instant disclosure may be used in methods of treating cancers such as, for example, biliary tract cancer, bladder cancer (including, e.g., urothelial carcinoma), brain cancer (including, e.g., glioblastoma and brain metastases from another cancer type), breast cancer (including, e.g., triple-negative breast cancer (TNBC), HER2-positive breast cancer, and HR+ breast cancer), cervical cancer, cholangiocarcinoma, colorectal cancer, endocrine cancer, endometrial cancer, epithelial cancer, esophageal cancer (including, e.g., gastroesophageal junction cancer, which can also be referred to as gastroesophageal junction adenocarcinoma), gastric cancer, head and/or neck cancer, hepatocellular carcinoma, lung cancer (including, e.g., non-small cell lung cancer (NSCLC)), melanoma, mesothelioma, nasopharyngeal cancer, neuroendocrine tumor, ovarian cancer, pancreatic cancer (including, e.g., pancreatic ductal adenocarcinoma (PDAC)), prostate cancer, rectal cancer, salivary gland cancer, sarcoma, small intestine cancer, testicular cancer, thyroid cancer, and/or uterine cancer.

In some embodiments, HER2-targeting radioligand therapeutics (HER2-targeting radiopharmaceuticals or HER2-targeting radiotherapies) of the instant disclosure are used in a method of treating a carcinoma arising from epithelial tissue.

In some embodiments, HER2-targeting radioligand therapeutics (HER2-targeting radiopharmaceuticals or HER2-targeting radiotherapies) of the instant disclosure are used in a method of treating a biliary tract cancer, bladder cancer (including, e.g., urothelial carcinoma), brain cancer (including, e.g., glioblastoma and brain metastases from another cancer type), breast cancer (including, e.g., triple-negative breast cancer (TNBC), HER2-positive breast cancer, and HR+ breast cancer), cervical cancer, cholangiocarcinoma, colorectal cancer, endocrine cancer, endometrial cancer, epithelial cancer, esophageal cancer (including, e.g., gastroesophageal junction cancer, which can also be referred to as gastroesophageal junction adenocarcinoma), gastric cancer, head and/or neck cancer, hepatocellular carcinoma, lung cancer (including, e.g., non-small cell lung cancer (NSCLC)), melanoma, mesothelioma, nasopharyngeal cancer, neuroendocrine tumor, ovarian cancer, pancreatic cancer (including, e.g., pancreatic ductal adenocarcinoma (PDAC)), prostate cancer, rectal cancer, salivary gland cancer, sarcoma, small intestine cancer, testicular cancer, thyroid cancer, and/or uterine cancer.

In particular embodiments, the HER2-targeting radioligand therapeutics are used in a method of treating bladder cancer, breast cancer, colorectal cancer, gastric cancer, gastroesophageal junction cancer, non-small cell lung cancer, or pancreatic ductal adenocarcinoma (PDAC).

In particular embodiments, the HER2-targeting radioligand therapeutics are used in a method of treating biliary tract cancer, bladder cancer, cervical cancer, endometrial cancer, ovarian cancer, or pancreatic cancer.

In particular embodiments, the HER2-targeting radioligand therapeutics are used in a method of treating breast cancer, non-small cell lung cancer, gastric cancer, or gastroesophageal junction cancer.

In an embodiment, HER2-targeting radioligand therapeutics of the present disclosure are used in methods of treating cancer. In certain embodiments, the cancer is a solid tumor. In certain embodiments, the cancer is characterized by HER2 over-expression (i.e., the cancer is HER2 positive). In certain embodiments, the cancer is characterized by HER2 expression. In certain embodiments, the cancer is characterized as HER2-low (i.e., the cancer is HER2-negative).

In certain embodiments, the cancer is classified as IHC 3+. In certain embodiments, the cancer is classified as IHC 2+. In certain embodiments, the cancer is classified as IHC 1+. In certain embodiments, the cancer is classified as IHC 0.

HER2 testing guidelines are described in, e.g., Wolff, A. C., Somerfield, M. R., Dowsett, M. & et, a., 2023. Human Epidermal Growth Factor Receptor 2 Testing in Breast Cancer: ASCO-College of American Pathologists Guideline Update. Journal of Clinical Oncology, 41 (22), pp. 3867-3872. In certain embodiments, the HER2 expression is measured by either IHC (Immunohistochemistry) or administration of a HER2-targeted radioimaging agent (such as, e.g., those disclosed herein). In certain embodiments, the cancer is breast cancer and the breast cancer is characterized as HER2-positive (HER2+). In certain embodiments, the breast cancer is characterized as HR+, HER2-low (HER2- or HER2-positive).

In certain embodiments, the cancer is breast cancer and the breast cancer is characterized as HER2-low. In certain embodiments, the breast cancer is characterized as HR+, HER2-low (HER2- or HER2-negative). In certain embodiments, the breast cancer is characterized as HR−, HER2-low (HER2- or HER2-negative).

In certain embodiments, the cancer is non-small cell lung cancer and the non-small cell lung cancer is characterized by HER2 expression. In certain embodiments, the non-small cell lung cancer is characterized as HER2+ (HER2≥1+ or HER2-positive). In certain embodiments, the non-small cell lung cancer is characterized as IHC≥1+.

In certain embodiments, the cancer is gastric cancer and the gastric cancer is characterized by HER2 expression. In certain embodiments, the cancer is gastric cancer and the gastric cancer is characterized as HER2-positive.

In certain embodiments, the cancer is gastroesophageal junction cancer and the gastroesophageal junction cancer is characterized by HER2 expression. In certain embodiments, the cancer is gastroesophageal junction cancer and the gastroesophageal junction cancer is characterized as HER2-positive.

In certain embodiments, the cancer is bladder cancer and the bladder cancer is characterized by HER2 expression. In certain embodiments, the cancer is bladder cancer and the bladder cancer is characterized as HER2-positive.

In some embodiments, methods of treating HER2-related diseases and disorders include co-administration of a disclosed radioligand with an additional therapy. In particular, when the HER2-related disease or disorder is cancer, methods may include combining a HER2-targeting radioligand therapeutic with one or more anti-cancer therapies (i.e., an anti-cancer therapeutic agent or a chemotherapeutic). Any known anticancer therapy may be combined with the radioligand of the present disclosure in order to provide curative, adjuvant, or palliative treatment to a subject in need thereof. For example, suitable anticancer therapies that may be combined with the presently disclosed radioligand therapeutic include, but are not limited to, surgery, anticancer drugs, PARP inhibitors, inhibitors of signaling pathways and mechanisms leading to repair of DNA single and double strand breaks, such as nuclear factor-kappa B signaling, immunomodulators, immune checkpoint inhibitors, antibodies capable of inducing antibody-dependent cellular cytotoxicity, T-cell or NK cell engagers, and cellular therapies using expanded autologous or allogeneic cells. One of ordinary skill in the art would be capable of discerning a suitable anticancer therapy to be used in combination with the radioligand therapeutic depending on a number of factors including, cancer type, stage, and location, as well as the general health and physical characteristics of the patient, e.g., weight, age, sex, etc.

The additional anticancer therapy may be administered concurrent with, prior to, or after administration of the HER2-targeting radioligand therapeutic. In some embodiments, the administration schedule involves administering the different agents in an alternating fashion, such as alternating days, weeks, or months. In other embodiments, the compounds may be delivered before and during, or during and after, or before and after treatment with one or more other anticancer therapies. In some embodiments, more than one additional anticancer therapy is administered to a subject. For example, the subject may receive the presently described HER2-targeting radioligand therapeutic, in combination with surgery and at least one other anticancer drug. Alternatively, the compound may be administered in combination with more than one additional anticancer drug or therapy.

In any of the foregoing methods, administration of the HER2-targeting radioligand therapeutic to the subject may be conducted parenterally, in particular, intravenously, but other routes of administration are not excluded. Other routes of administration include, but are not limited to, subcutaneous, intramuscular, intraperitoneal, transdermal, topical, buccal, or oral routes. In certain embodiments, the radioligand therapeutic may be administered as close to the disease site as possible, such as in the diseases tissue, surrounding tissue, or nearby blood vessels.

The HER2-targeting radioligand therapeutic may be dosed according to the total radiation to be administered to the subject in need thereof, and as described herein.

In some embodiments, the HER2-targeting radioligand therapeutic is administered on alternate days, weeks, or months. For example, HER2-targeting radioligand therapeutic described herein may be administered once every two days, or every three days, or every four days, or every five days, or every six days, or every week, or every month. The HER2-targeting radioligands described herein may be administered once every two weeks, or every three weeks, or every four weeks, or every five weeks, or every six weeks, or every seven weeks, or every eight weeks, or every nine weeks, or every ten weeks, or every eleven weeks, or every twelve weeks.

In some embodiments, the HER2-targeting radioligand is administered once about every 2 weeks to 10 weeks. In some embodiments, the HER2-targeting radioligand is administered once about every 2 weeks to 6 weeks. In some embodiments, the HER2-targeting radioligand is administered once about every 2 weeks to 4 weeks. In some embodiments, the HER2-targeting radioligand is administered once about every 3 weeks to 10 weeks. In some embodiments, the HER2-targeting radioligand is administered once about every 3 weeks to 6 weeks. In some embodiments, the HER2-targeting radioligand is administered once about every 3 weeks to 4 weeks. In some embodiments, the HER2-targeting radioligand is administered once about every 4 weeks to 6 weeks. In some embodiments, the HER2-targeting radioligand is administered once about every 5 weeks to 6 weeks. In some embodiments, the HER2-targeting radioligand is administered once about every 6 weeks to 8 weeks. In some embodiments, the HER2-targeting radioligand is administered once about every 6 weeks to 7 weeks.

In some embodiments, the present disclosure provides a method of treating a subject afflicted with a disease or disorder using the HER2-targeting therapeutics described herein, wherein the treatment is radionuclide therapy. Radionuclide therapy is based on different forms of radiation emitted by a radionuclide, including, but not limited to, radiation of photons, radiation of electrons, such as, for example, β-particles and auger-electrons, radiation of protons, radiation of neutrons, radiation of positrons, radiation of α-particles or an ion beam. Accordingly, depending on the kind of particle or radiation emitted by the radionuclide, radionuclide therapy can be distinguished as photon radionuclide therapy, electron radionuclide therapy (e.g., β-particle radionuclide therapy), proton radionuclide therapy, neutron radionuclide therapy, positron radionuclide therapy, α-particle radionuclide therapy, or ion beam radionuclide therapy. Methods of the present disclosure that utilize radionuclide therapy for treatment of a subject may rely on any one or more of these forms of radiotherapy. In particular, the HER2-targeting radioligand therapeutic of the present disclosure may be used to provide any one or more of the above forms of radionuclide therapy to a subject in need thereof. In certain embodiments, α-emitting radionuclide therapy may be useful. In certain other embodiments, β-emitting radionuclide therapy may be useful.

In some embodiments, methods may include the use of certain techniques or systems commonly used in a clinical setting to accelerate and/or increase the effectiveness of the radiation therapy. For example, as oxygen is known in the art to be a potent radiosensitizer by readily forming free radicals, the presently described radioligand may be used in conjunction with high-pressure oxygen tanks, blood substitutes having increased oxygen content, and hypoxic cell radiosensitizers in some cases.

The compounds disclosed herein, including the compounds of Formula (I), (Ia), (Ib), (Ic), or (Id), or a pharmaceutically acceptable salt or solvate thereof, exhibit strong binding to human and/or mouse HER2, i.e., exhibit a dissociation constant (K_{D})) of about 1000 nM, about 500 nM, or about 200 nM or less as measured by surface plasmon resonance (SPR) at a temperature of 25° C. (see, e.g., the method described particularly in Section 11.1 and corresponding K_{D} values in Table 11.1).

In some embodiments, any one of the compounds as disclosed herein exhibit a K_{D} between about 1000 nM and about 0.001 nM as measured by SPR at a temperature of 25° C. In some embodiments, any one of the compounds as disclosed herein exhibit a K_{D} between about 500 nM and about 0.001 nM as measured by SPR at a temperature of 25° C. In some embodiments, any one of the compounds as disclosed herein exhibit a K_{D} between about 200 nM and about 0.001 nM as measured by SPR at a temperature of 25° C.

The compounds disclosed herein, including the compounds of Formula (I), (Ia), (Ib), (Ic), or (Id), or a pharmaceutically acceptable salt or solvate thereof, also exhibit high potency against HER2 (inhibit human and/or mouse HER2 enzymatic activity), i.e., exhibit an IC₅₀ for human HER2 of about 1000 nM or less, about 500 nM or less, or about 200 nM or less as measured by an Enzymatic HER2 competition assay (see, e.g., the method described particularly in Section 11.1 and corresponding IC₅₀ values in Table 11.1).

In some embodiments, any one of the compounds as disclosed herein exhibit an IC₅₀ for human HER2 between about 1000 nM and about 0.001 nM as measured by an Enzymatic HER2 competition assay. In some embodiments, any one of the compounds as disclosed herein exhibit an IC₅₀ for human HER2 between about 500 nM and about 0.001 nM as measured by an Enzymatic HER2 competition assay. In some embodiments, any one of the compounds as disclosed herein exhibit an IC₅₀ for human HER2 between about 200 nM and about 0.001 nM as measured by an Enzymatic HER2 competition assay.

In some embodiments, any one of the compounds disclosed herein are selective for HER2 over Herstatin and other members of the ErbB family (such as, e.g., EGFR, HER3, and HER4).

### Methods of Imaging

Also provided herein is a method of imaging a disease or disorder associated with human epidermal growth factor receptor 2 using the disclosed HER2-targeting radioligands. In some aspects, the method includes administering a detectably effective amount (an amount effective for imaging) of a radioligand (i.e., a radioimaging agent) of the present disclosure, or a pharmaceutical composition thereof, to a subject.

In some embodiments, the method includes imaging one or more cells, tissues, or organs, including, but not limited to, kidney tissue, prostate tissue, brain tissue, vascular tissue, and tumor tissue.

The HER2-targeting compounds described herein are suitable for imaging any physiological process or feature in which HER2 is involved, such as for identifying areas of tissues or targets which exhibit or express high concentrations (e.g., over-expression) of HER2. Exemplary physiological processes in which HER2 is involved include, but are not limited to, proliferation diseases, tissue remodeling and/or chronic inflammation, and endocrinological disorders.

Provided herein is a method of identifying a subject suitable for treatment with a HER2-targeting radioligand therapeutic comprising administering to the subject a HER2-targeting radioimaging agent and imaging the subject (by, e.g., PET or PET/CT) to determine whether the subject has HER2 expressing cancer. In certain embodiments, the HER2-targeting radioimaging agent comprises a HER2-targeting compound disclosed herein radiolabeled with a radionuclide suitable for radioimaging (e.g., by PET and/or PET/CT and/or SPECT/CT), such as, for example, ¹¹¹In, ⁶⁸Ga or ¹⁸F. In certain embodiments, the HER2-targeting radioligand therapeutic comprises a HER2-targeting compound disclosed herein radiolabeled with a radionuclide suitable for therapy, such as, for example, ¹⁷⁷Lu, ²²⁵Ac, or ¹⁶¹Tb. In some embodiments, the method includes imaging one or more cells, tissues, or organs implicated in a proliferation disease. In some embodiments, the proliferation disease is cancer. For example, one or more cells, tissues, or organs suitable for imaging with the radioligand peptides described herein may be implicated in cancers such as, for example, breast cancer, colorectal cancer, epithelial cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.

In some embodiments, the method includes imaging one or more cells, tissues, or organs implicated in tissue remodeling and/or chronic inflammation, including, but not limited to, fibrotic disease, wound healing, keloid formation, osteoarthritis, rheumatoid arthritis, and relating disorders involving cartilage degradation.

In some aspects, methods of imaging one or more cells, tissues, or organs with HER2-targeting radioligands of the present disclosure further include detecting and/or assessing a disease or disorder in the subject. Detecting and/or assessing a disease or disorder may be useful in diagnosis, prognosis, and prediction of a disease or disorder in the subject. For example, HER2-targeting radioligands disclosed herein may be used to diagnose a disease or disorder afflicting the subject, to determine the subject's risk of developing a disease or disorder, to assess the evolution of a disease or disorder in the subject, or to predict a subject's response to a certain therapy given to treat the disease or disorder.

In some embodiments, the HER2-targeting radioligands are detectable by positron emission tomography (PET) or single photon emission computed tomography (SPECT). In some embodiments, the HER2-targeting radioligands are detectable by scintigraphy.

The HER2-targeting radioligand or a pharmaceutical composition thereof may be administered to the subject for imaging purposes via parenteral administration, but other routes of administration are not excluded. Other routes of administration include, but are not limited to, subcutaneous, intravenous, intramuscular, intraperitoneal, transdermal, or oral routes. The amount of a radioligand described herein, or a pharmaceutically acceptable salt or composition thereof to be administered to a subject may be determined by a person of skill in the art taking into account the disease or condition being treated, including its locale, and factors including age, weight, sex, and the like.

### Theragnostic Methods

The present disclosure further provides a method to both image and treat or prevent a disease or disorder associated with human epidermal growth factor receptor 2 using the disclosed HER2-targeting ligands and radioligands. As used herein, a method of imaging and treating a disease or disorder may be referred to as a theragnostic method.

In some embodiments, the theragnostic method includes administering a diagnostically effective and therapeutically effective dose of a HER2-targeting compound described herein to a subject in need thereof. In some embodiments, a HER2-targeting compound may be used to image one or more tissues, cells, or organs implicated in a HER2-related disease and to treat the HER2-related disease in those tissues, cells, or organs. In particular, a theragnostic method may be used for both imaging and treatment of cancer in a subject in need thereof.

In some embodiments, one HER2-targeting compound is used in the theragnostic method described herein (i.e., the same HER2-targeting radioligand is used in both imaging and therapeutic methods). Accordingly, in some embodiments, the HER2-targeting compound is both diagnostically and therapeutically active.

The HER2-targeting compound used in a theragnostic method may be complexed to one radionuclide that is diagnostically and therapeutically active, or more than one radionuclide where at least one of the more than one radionuclides is diagnostically active and at least one of the more than one radionuclide is therapeutically active. In some embodiments, the diagnostically active radionuclide is a radiohalogen and the therapeutically radionuclide is a radionuclide other than a radiohalogen. In other embodiments, the therapeutically and diagnostically active radionuclides are radiohalogens. In yet other embodiments, the therapeutically and diagnostically active radionuclides are radionuclides other than radiohalogens.

Alternatively, in some embodiments, more than one HER2-targeting compounds are used in a theragnostic method descried herein. In such embodiments, a diagnostically active HER2-targeting radioligand is used first to diagnose or image the disease or disorder and a therapeutically active HER2-targeting radioligand is subsequently used to treat the disease or disorder. For example, when the HER2-related disease or disorder is cancer, a diagnostically active HER2-targeting radioligand may initially be used to identify and localize the primary tumor mass, and to determine potential local and distant metastasis, and after imaging/diagnosis, a therapeutically active HER2-targeting radioligand (comprising a therapeutically active radionuclide) may be administered as described previously to treat the tumor.

When more than one HER2-targeting compounds are used in a theragnostic method, each HER2-targeting compound may be complexed to one or more than one radionuclide. In some embodiments, a diagnostically active HER2-targeting compound is complexed to one diagnostically active radionuclide. In some embodiments, a diagnostically active HER2-targeting compound is complexed to more than one diagnostically active radionuclide. In some embodiments, a therapeutically active HER2-targeting compound is complexed to one therapeutically active radionuclide. In some embodiments, a therapeutically active HER2-targeting compound is complexed to more than one therapeutically active radionuclide.

In some embodiments of the theragnostic method described herein, the diagnostic/imaging agent is a HER2-targeting imaging agent described in the art, and the therapeutic agent is a radiolabeled compound described herein (e.g., a radiolabeled compound of any one of Formulae (I), (Ia), (Ib), (Ic), (Id), and (I-i)). In other embodiments of the theragnostic method described herein, the diagnostic/imaging agent is a radiolabeled compound described herein (e.g., a radiolabeled compound of any one of Formulae (I), (Ia), (Ib), (Ic), (Id), and (I-i)) and the therapeutic agent is a HER2-targeting therapeutic agent described in the art.

The dosage and administration schedule of a HER2-targeting compound used for theragnostic purposes may be the same as the dosage and administration schedules described above, e.g., a diagnostically active HER2-targeting compound used in a theragnostic method may be dosed and administered as described for a method of imaging and therapeutically active HER2-targeting compound used in a theragnostic method may be dosed and administered as described above for a method of treatment. A therapeutically and diagnostically active HER2-targeting compound may be dosed and administered according to either of the above methods. The dose and administration of a radioligand described herein, or a pharmaceutically acceptable salt or composition thereof to be administered to a subject may be determined by a person of skill in the art taking into account the disease or condition being diagnosed and treated, including its locale, and factors including age, weight, sex, and the like.

### Kits

The present disclosure also includes pharmaceutical kits useful, for example, in the imaging, diagnosis, or treatment of a HER2-related disease or disorder (such as, e.g., cancer) which include one or more containers containing a pharmaceutical composition comprising a therapeutically effective amount of a HER2-targeting ligand and/or radioligand of the disclosure. Such kits can further include one or more various kit components such as, for example, containers with one or more pharmaceutically acceptable carriers, additional containers (e.g., those suitable for handling radioactive waste), and/or instructions, inserts, or labels, indicating quantities of components to be administered, guidelines for administration, and/or guidelines for mixing the components.

### Equivalents and Scope

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby.

It is to be further understood that resort may be had to various other embodiments, modifications, and equivalents thereof which may suggest themselves to those skilled in the art without departing from the spirit of the present disclosure and/or scope of the appended claims.

### EXAMPLES

### Example I. Synthesis and Characterization of Cyclic Peptides and Compounds Comprising them

### 1 Abbreviations and General Procedures

### 1.1 Abbreviations

| | |
|---|---|
| Å | angstrom |
| AA | amino acid |
| AAZTA5 | 1,4-bis(carboxymethyl)-6-[bis(carboxymethyl)]amino-6- |
| | [pentanoic-acid]perhydro-1,4-diazepine |
| Ac | acetyl |
| Ac₂O | acetic anhydride |
| ACN | acetonitrile |
| AcOEt | ethyl acetate |
| AcOH | acetic acid |
| Ac₂O | acetic anhydride |
| All | allyl (2-propenyl) |
| Alloc | allyloxycarbonyl |
| aq. | aqueous |
| bA, b-Ala, or | β-alanine |
| Beta-Ala | |
| BB | building block |
| Boc | tert-butoxycarbonyl |
| Boc₂O | Di-tert-butyl dicarbonate |
| ClAcOH | chloroacetic acid |
| CPME | cyclopentyl methyl ether |
| DAD | diode-array detection |
| DBU | 1,8-diazabicyclo[5.4.0]undec-7-ene |
| DCC | N,N′-dicyclohexylcarbodiimide |
| DCE | 1,2-dichloroethane |
| DCM | dichloromethane |
| DIC | N,N′-diisopropylcarbodiimide |
| DIEA, DIPEA | N,N-diisopropylethylamine |
| DMA | N,N-dimethylacetamide |
| DMAP | 4-(dimethylamino)pyridine |
| DMF | N,N-dimethylformamide |
| DMSO | dimethylsulfoxide |
| DODT | 3,6-dioxa-1,8-octanedithiol |
| DOTA | 2,2′,2″,2″′-(1,4,7, 10-tetraazacyclododecane-1,4,7,10- |
| | tetrayl)tetraacetic acid |
| DOTAGA | 2-(4,7,10-tris(carboxymethyl)-1,4,7,10- |
| | tetraazacyclododecan-1-yl)pentanedioic acid |
| EDCI | 1-ethyl-3-(3-dimethylaminopropyl)carbodiimide |
| eq. | equivalent(s) |
| ESI | electrospray ionization |
| EtOAc | ethyl acetate |
| Fmoc | 9-fluorenylmethyloxycarbonyl |
| Fmoc-OSu | 9-fluorenylmethyl-N-hydroxysuccinimide |
| g | gram |
| HATU | O-(7-azabenzotriazol-1-yl)-1,1,3,3-teramethyluronium |
| | hexafluorophosphate |
| HCTU | O-(1H-6-chlorobenzotriazol-1-yl)-1,1,3,3- |
| | tetramethyluronium hexafluorophosphate |
| HFIP | 1,1,1,3,3,3-hexafluoropropan-2-ol |
| HMPA | 2-(4-(hydroxymethyl)-3-methoxyphenoxy)acetic acid |
| HMPB | 4-(4-hydroxymethyl-3-methoxyphenoxy)butyric acid |
| HOAt | 3H-[1,2,3]triazolo[4,5-b]pyridin-3-ol |
| HOBt | 1-hydroxybenzotriazole |
| HPLC | high-performance liquid chromatography |
| hr | hour(s) |
| IPA | isopropanol |
| L | liter |
| LC | liquid chromatography |
| LCMS | liquid chromatography mass spectrometry |
| M | molar |
| m/z | mass to charge ratio |
| MBHA | 4-methylbenzhydrylamine |
| MeOH | methanol |
| mg | milligram(s) |
| min | minute(s) |
| mL | milliliters(s) |
| mM | millimolar |
| mmol | millimole(s) |
| Mol | mole(s) |
| 3-MPA | 3-mercaptopropionic acid |
| MPE | 3-methyl-3-pentyl |
| MS | mass spectrometry |
| MTBE | tert-butyl methyl ether |
| NHS | N-hydroxysuccinimide |
| NIS | N-iodosuccinimide |
| NMP | N-methyl-2-pyrrolidone |
| NMR | nuclear magnetic resonance |
| NODAGA | 2-(4,7-bis(carboxymethyl)-1,4,7-triazonan-1- |
| | yl)pentanedioic acid |
| NOTA | 2,2′,2″-(1,4,7-triazonane-1,4,7-triyl)triacetic acid |
| Oxyma Pure ® | ethyl cyano(hydroxyimino)acetate |
| Pbf | 2,2,4,6,7-pentamethyldihydrobenzofuran-5-sulfonyl |
| Pd(PPh₃)₄ | Tetrakis(triphenylphosphane)palladium(0) |
| PE | petroleum ether |
| PG | protecting group |
| Ph | phenyl |
| PMB | p-methoxybenzyl |
| PS | polystyrene |
| PyAOP | (7-azabenzotriazol-1-yloxy)tripyrrolidinophosphonium |
| | hexafluorophosphate |
| PyOxim | (ethyl cyano(hydroxyimino)acetato-O²)-tri-(1- |
| | pyrrolidinyl)-phosphonium hexafluorophosphate |
| QTof | quadrupole time of flight |
| RP | reversed-phase |
| Rt, RT | room temperature |
| Sar | sarcosine |
| sat. | saturated |
| SPPS | solid phase peptide synthesis |
| Cy5 | Cyanine5 |
| T3P | propanephosphonic acid anhydride |
| TBAF | tetra-n-butylammonium fluoride |
| TBS | tert-butyldimethylsilyl |
| TBTU | 2-(1H-benzotriazole-1-yl)-1,1,3,3-tetramethylaminium |
| | tetrafluoroborate |
| tBu | tert-butyl |
| TCEP | tris(2-carboxyethyl)phosphine hydrochloride salt |
| TEA, Et₃N | triethylamine |
| TFA | trifluoroacetic acid |
| TFAA | trifluoroacetic anhydride |
| THF | tetrahydrofuran |
| TIS | triisopropylsilane |
| TMSOTf | trimethylsilyl trifluoromethanesulfonate |
| TPTU | O-(1,2-dihydro-2-oxo-1-pyridyl)-N,N,N′,N′- |
| | tetramethyluronium tetrafluoroborate |
| t_{R} | retention time |
| Trt | trityl, triphenylmethyl |
| UPLC | ultra-performance liquid chromatography |
| UV | ultraviolet |
| μg | microgram(s) |
| μL | microliter(s) |
| μM | micromolar |
| μmol | micromole(s) |
| wt | weight |

### 1.2 General Procedures

Unless otherwise noted, reagents and solvents were used as received from commercial suppliers. Proton nuclear magnetic resonance (NMR) spectra were obtained on a Varian spectrometer at 400 MHz or a Bruker spectrometer at 400 MHz or 600 MHz. Chemical shifts are given in ppm (d) and coupling constants, J, are reported in Hertz. Tetramethylsilane (TMS) or the solvent peak was used as an internal standard. Only the protons visible in ¹H NMR were listed. Signals hidden under the solvent peaks were not included. The linear peptides were assembled by Fmoc-based solid phase peptide synthesis using typically peptide synthesizers, namely the Liberty Blue and Liberty Prime from CEM or by manual coupling (see Section 1.3). The syntheses were done at RT or with heating starting from Fmoc-Rink Amide ProTide resin, Fmoc-Rink Amide AM resin, Fmoc-Sieber resin or Fmoc-Lys (Boc)-Wang resin (see Section 10.1, Table 10.1). Fmoc-protected amino acids with appropriate protecting groups on side chains were used (commercially available or synthesized as described, see Section 10.2, Table 10.2). In some instances, other amino acids or building blocks were used (see Section 10.3, Table 10.3). Amino acids were coupled using a variety of coupling reagents such as HATU, PyAOP, DIC/Oxyma Pure® and, depending on the requirements, a base such as DIPEA. Fmoc was typically removed using 4-methylpiperidine, piperidine or pyrrolidine. Cleavage of the fully protected peptides from the resin was achieved with a mixture of TFA/DCM (e.g. 1% TFA in DCM). The linear peptides were cyclized directly or in some instances purified prior to cyclization. The amide bond cyclization was typically performed in DMF or DCM using HATU/2,6-lutidine. Other conditions used for cyclization were e.g. PyAOP/Et₃N, or PyAOP/DIPEA in DCM or T3P/DIPEA in DMF. Thioether macrocyclization was typically performed in ACN/water using a base (e.g. DIPEA or Et₃N). Disulfide macrocyclization was typically performed in DMSO/water (1/4). Concomitant removal of the protecting groups and cleavage from the resin was achieved by using mixtures of TFA/H₂O/DODT/TIS (e.g. 92.5/2.5/2.5/2.5). Selective removal of the allyl and alloc protecting groups was achieved using catalytic amounts of Pd(PPh₃)₄) in the presence of a scavenger (e.g. phenylsilane, triethylsilane or 1,3-Dimethylbarbituric acid) in a solvent (e.g., DCM). Chelator coupling was performed using chelator reagents such as DOTA-NHS ester, NODAGA-NHS ester, NOTA-NHS ester, (R)-DOTAGA-anhydride, p-SCN-Bn-DOTA or/Bu-protected AAZTA5, in the presence of a base (e.g., DIPEA), and if required coupling reagents such as HATU and HOAt. Labelling with natural ¹⁷⁵Lu, ^{nat}Ga, ¹³⁹La or Al^{nat}F was performed in various buffers (e.g. ammonium acetate or sodium acetate buffers) at pH 3.5-6.0 at 80° C.-100° C., in the presence or absence of DMF.

The final products and intermediates were purified by the preparative reversed-phase HPLC methods described in Section 1.4.

The products and intermediates were analyzed by the analytical methods described in Section 1.5 and Section 1.6.

### 1.3 SPPS Methods

### 1.3.1 SPPS Using Automated Peptide Synthesizer

The resin was swollen in the solvent (e.g., DMF or DCM) and then loaded onto the peptide synthesizer (e.g. Liberty Blue™ or Liberty PRIME™ from CEM Inc.) for the SPPS. Fmoc was typically removed using 4-methylpiperidine, piperidine or pyrrolidine. Coupling was accomplished by addition of the Fmoc-amino acid, DIC and Oxyma Pure® or Oxyma Pure®+DIPEA. The coupling and deprotection steps were repeated until the desired polypeptide was obtained. Sometimes the SPPS sequence was interrupted after a cycle and a Fmoc deprotection was performed (e.g. before a manual coupling). After the final coupling was completed, the resin was washed with an appropriate solvent (e.g. DMF, DCM) to provide the protected peptide on resin. The specific conditions used for the automated SPPS are described in the experimental sections below.

### 1.3.2 SPPS by Manual Coupling

The resin was swollen in the solvent (e.g., DMF or DCM). The Fmoc amino acid was coupled using a variety of coupling reagents such as HATU, PyAOP, DIC/Oxyma Pure® and depending on the requirements a base such as DIPEA or Et₃N. After a pre-activation time the solution was added to the resin. The reaction was shaken at RT upon completion of the reaction. The solution was drained, and the resin washed with an appropriate solvent (e.g. DMF, DCM). Fmoc was typically removed using 4-methylpiperidine, piperidine or pyrrolidine. The specific conditions used for the manual SPPS are described in the experimental sections.

### 1.4 Preparative HPLC Purification Methods

The final products and intermediates were purified by preparative reversed-phase HPLC, using columns of different sizes and with varying flow rates, depending on the amount of material to be purified.

The following columns were used:
XSelect® Peptide CSH C18, OBD™ Prep Column, 130 Å, 5 μm, 30 mm×100 mm
XSelect® Peptide CSH C18, OBD™ Prep Column, 130 Å, 5 μm, 30 mm×250 mm
XSelect® Peptide CSH C18, OBD™ Prep Column, 130 Å, 5 μm, 19 mm×150 mm
XBridge® Peptide CSH C18, OBD™ Prep Column, 130 Å, 5 μm, 30 mm×150 mm
XBridge® C18, OBD™ Prep Column, 130 Å, 5 μm, 30 mm×150 mm
XBridge® C18, OBD™ Prep Column, 130 Å, 5 μm, 50 mm×250 mm
XBridge® C18, OBD™ Prep Column, 130 Å, 5 μm, 50 mm×150 mm
XBridge® C18, OBD™ Prep Column, 130 Å, 5 μm, 19 mm×150 mm
Kinetex® EVO C18, OBD™ Prep Column, 130 Å, 5 μm, 30 mm×150 mm

The following mobile phases were used:
Eluent A: Water+0.1% TFA and eluent B: ACN
Eluent A: Water+0.1% TFA and eluent B: ACN+0.1% TFA
Eluent A: Water+1% AcOH and eluent B: ACN+1% AcOH
Eluent A: Water+0.08% NH₄HCO₃ and eluent B: ACN

Gradients were designed based on the specific requirements of the separation problem. Pure products were lyophilized from ACN/H₂O and obtained, depending on the used eluents, as a free base or the corresponding salt (e.g. trifluoroacetate, acetate).

### 1.5 Analytical Methods (Peptides)

### 1.5.1 LCMS Method AP-1

Waters Acquity UPLC-MS system/QTof MS (ESI): Eluent A: Water+0.05% TFA; Eluent B: ACN+0.04% TFA; Column temperature: 80° C.; Flow: 0.5 mL/min; Column: Acquity UPLC CSH C18, 1.7 μm, 2.1×100 mm; Gradient: hold 5% B for 0.2 min; from 5 to 98% B in 9.2 min; hold 98% B for 0.4 min.

### 1.5.2 LCMS Method AP-2

Waters Acquity UPLC-MS system/QTof MS (ESI): Eluent A: Water+0.05% TFA; Eluent B: ACN+0.04% TFA; Column temperature: 80° C.; Flow: 0.5 mL/min; Column: Acquity UPLC CSH C18, 1.7 μm, 2.1×100 mm; Gradient: hold 0% for 0.3 min; from 0 to 40% B in 8.7 min; from 40 to 98% B in 0.5 min.

### 1.5.3 LCMS Method AP-3

Shimadzu LC/MS system/Single Quadrupole (ESI): Eluent A: Water+0.025% TFA; Eluent B: ACN+0.025% TFA; Column temperature: 60° C.; Flow: 0.5 mL/min; Column: Kinetex EVO C18 2.6 um, 2.1 mm×150 mm, 100 Å (with a guard cartridge 2.1 mm ID); Gradient: hold 0% B for 0.3 min; from 20 to 60% B in 7.15 min; from 60 to 95% B in 0.3 min; hold 95% for 1.55 min; from 95 to 20% B in 0.01 min; hold 20% for 3.49 min. hold 20% for 3.49 min.

### 1.6 Analytical Methods (Building Blocks)

### 1.6.1 LCMS Method AB-1

Waters Acquity UPLC-MS system/MS (ESI): Eluent A: Water+4.76% isopropanol+0.05% formic acid+3.75 mM ammonium acetate; Eluent B: isopropanol+0.05% formic acid; Column temperature: 80° C.; Flow: 1.0 mL/min; Column: CORTECS C18+, 2.1×50 mm, 2.7 μm; Gradient: 1% to 50% B in 1.4 min; 50 to 98% B in 0.3 min.

### 1.6.2 LCMS Method AB-2

Waters Acquity UPLC-MS system/MS (ESI): Eluent A: Water+0.025% TFA; Eluent B: ACN+0.025% TFA; Column temperature: 60° C.; Flow: 1.0 mL/min; Column: ACQUITY UPLC BEH C18 Column 1.7 μm 2.1×100 mm; Gradient: 5% to 95% B in 5.56 min; 95% B for 1.66 min.

### 1.6.3 LCMS Method AB-3

Agilent 1260 HPLC system/MS (ESI): Eluent A: Water+0.1% TFA; Eluent B: ACN+0.1% TFA; Column temperature: 40° C.; Flow: 1.2 mL/min; Column: Poroshell 120 EC-C18, 2.7 μm 4.6×50 mm; Gradient: 5% to 95% B in 5 min; 95% B for 2 min.

### 2 Assembly of Cyclic Peptides (“A” Examples)

### 2.1 Examples Synthesized Using Rink Amide Resin R1 or R2

### 2.1.1 Synthesis of (3S,6S,9S,12S,15S,18S,21S,24S,27S)-9-((1H-indol-3-yl)methyl)-27-acetamido-N—((S)-1-amino-3-(4-fluorophenyl)-1-oxopropan-2-yl)-6-cyclopropyl-12-((5-hydroxy-1H-indol-3-yl)methyl)-15,21-bis(hydroxymethyl)-24-((6-hydroxypyridin-3-yl)methyl)-N,4,16,19-tetramethyl-5,8,11,14,17,20,23,26,30-nonaoxo-18-phenethyl-1,4,7,10,13,16,19,22,25-nonaazacyclotriacontane-3-carboxamide (Example A1)

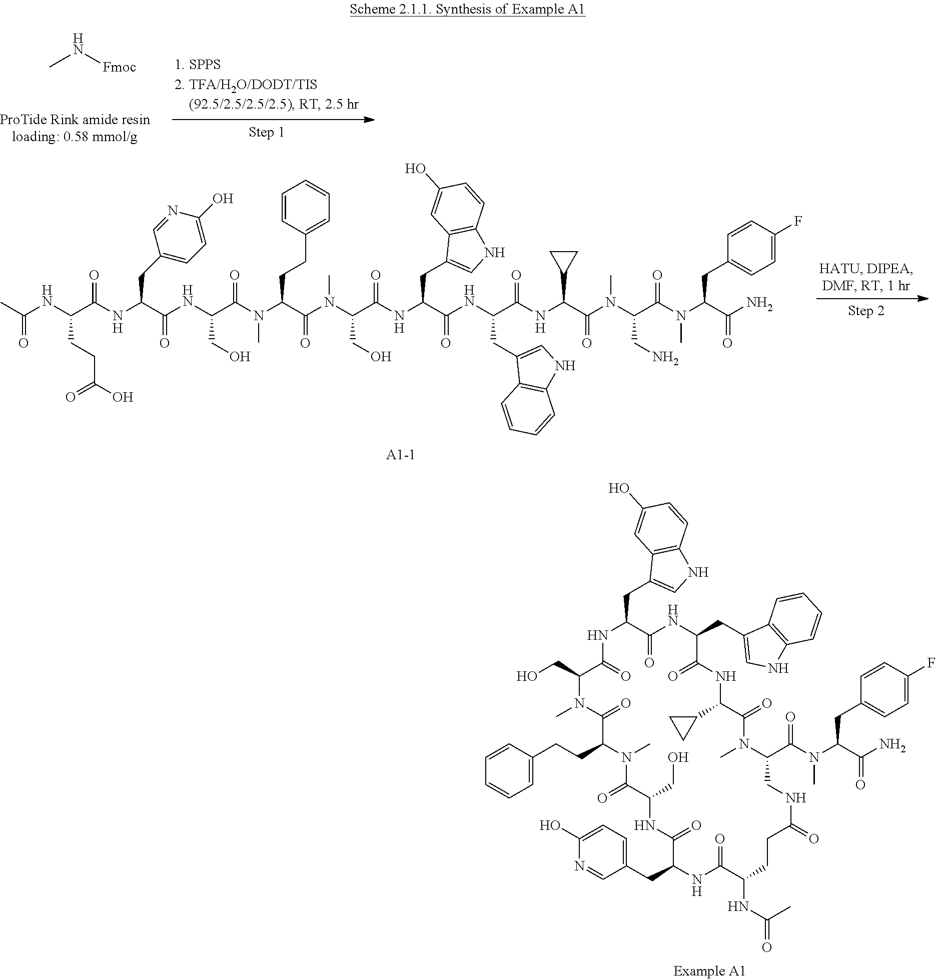

### Step 1. A1-1

Step 1-1: The assembly of the linear peptide was done on the CEM Liberty Prime Synthesizer using commercial Rink amide resin R1 (loading 0.58 mmol/g, 172 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.

After the assembly of the linear peptide the resin was washed with DMF (5×) and DCM (5×). The amino acids and coupling methods are summarized in Table 2.1.

TABLE 2.1

| Synthesis | Amino acid | | |
|---|---|---|---|
| cycle | residue^{a} | Amino acid | Method |
| 1 | A¹⁰ | Fmoc-L-N-Me-Phe(4F)-OH | A |
| 2 | A⁹ | Fmoc-L-N-Me-Dap(Boc)-OH | B |
| 3 | A⁸ | Fmoc-L-CyclopropylGly-OH | B |
| 4 | A⁷ | Fmoc-L-Trp(Boc)-OH | A |
| 5 | A⁶ | Fmoc-L-Trp(5-OH)-OH | A |
| 6 | A⁵ | Fmoc-L-N-Me-Ser(tBu)-OH | B |
| 7 | A⁴ | Fmoc-L-N-Me-Homo-Phe- | B |
| | | OH | |
| 8 | A³ | Fmoc-L-Ser(tBu)-OH | B |
| 9 | A² | Fmoc-L-3PyA(6OH)-OH | A |
| 10 | A¹ | Fmoc-L-Glu(OtBu)-OH | A |
| 11 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (5 mL) and the resin was shaken at RT for 2 hr. The crude peptide was precipitated with cold diethyl ether (40 mL). The suspension was centrifuged, and the solvent was decanted. The product was washed with another portion of cold diethyl ether (30 mL). The suspension was centrifuged, and the solvent was decanted. The crude peptide was dried at RT under vacuo, and the product was isolated by preparative RP HPLC (Column: SunFire C18 OBD™ Prep Column, 100 Å, 5 μm, 30 mm×150 mm; Eluent A: H₂O+0.1% TFA and eluent B: ACN). Pure fractions were combined and lyophilized to afford A1-1 (28.5 mg, 16 μmol, 20% yield, TFA salt) as a white solid. LCMS Method AP-1, t_{R}=3.43 min, [M+H]⁺=1480.7.

### Step 2. Example A1

A1-1 (28.5 mg, 1 eq., 17.9 μmol TFA salt) was dissolved in DMF (3 mL). Then HATU (8.15 mg, 1.2 eq., 21.4 μmol), followed by DIPEA (6.93 mg, 9.3 μL, 3 eq., 53.6 μmol) were added. The reaction mixture was stirred at RT for 3 hr, then quenched with ACN/water (1/1). The quenched reaction mixture was directly purified, and the product was isolated by preparative RP HPLC (Column: SunFire C18 OBD™ Prep Column, 100 Å, 5 μm, 30 mm×100 mm; Eluent A: H₂O+0.1% TFA and eluent B: ACN). Pure fractions were combined and lyophilized to afford Example A1 (5.3 mg, 3.4 μmol, 19% yield) as a white solid. LCMS Method AP-1, t_{R}=4.25 min, [M+H]⁺=1462.7.

The compounds in Table 2.2 were synthesized in analogy to Example A1.

TABLE 2.2
Examples synthesized in analogy to Example A1 (Scheme 2.1.1).

| Ex. No. | Structure/Example Name | LCMS |
|---|---|---|
| A2 (SEQ ID NO: 1) | 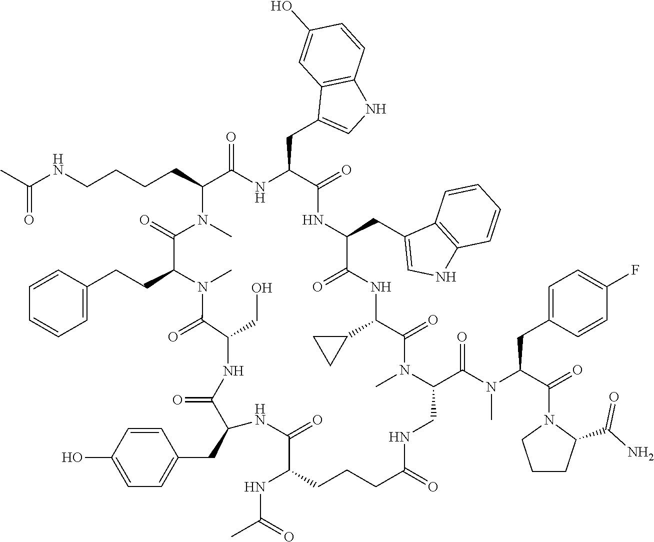 | Method AP-1 t_{R} = 4.65 min [M + 2H]²⁺ = 828.9 |
| | (3S,6S,9S,12S,15S,18S,21S,24S,27S)-9-((1H-indol-3-yl)methyl)-27-acetamido- | |
| | 15-(4-acetamidobutyl)-N-((S)-1-((S)-2-carbamoylpyrrolidin-1-yl)-3-(4- | |
| | fluorophenyl)-1-oxopropan-2-yl)-6-cyclopropyl-12-((5-hydroxy-1H-indol-3- | |
| | yl)methyl)-24-(4-hydroxybenzyl)-21-(hydroxymethyl)-N,4,16,19-tetramethyl- | |
| | 5,8,11,14,17,20,23,26,31-nonaoxo-18-phenethyl-1,4,7,10,13,16,19,22,25- | |
| | nonaazacyclohentriacontane-3-carboxamide | |
| A3 | 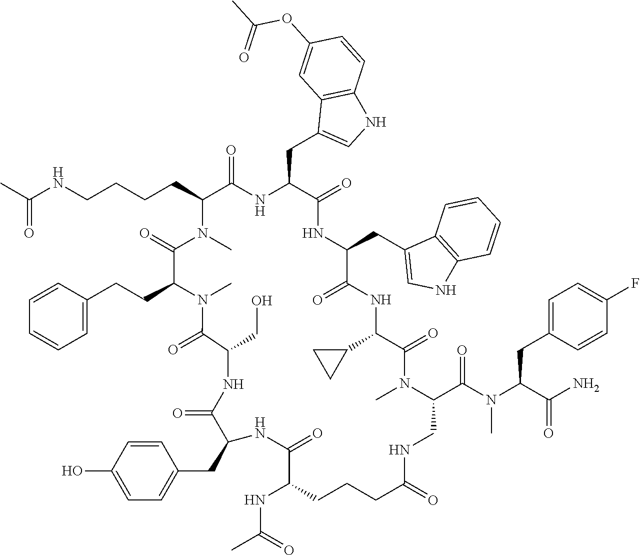 | Method AP-1 t_{R} = 4.93 min [M + H]⁺ = 1600.8 |
| | Note: The acetyl group at the 5′ position of the tryptophan | |
| | may rather be at the NH of the corresponding tryptophan. | |
| | This is due to capping performed after incorporation of the | |
| | unprotected W(5OH). | |
| | 3-(((3S,6S,9S,12S,15S,18S,21S,24S,27S)-9-((1H-indol-3-yl)methyl)-27- | |
| | acetamido-15-(4-acetamidobutyl)-3-(((S)-1-amino-3-(4-fluorophenyl)-1- | |
| | oxopropan-2-yl)(methyl)carbamoyl)-6-cyclopropyl-24-(4-hydroxybenzyl)-21- | |
| | (hydroxymethyl)-4,16,19-trimethyl-5,8,11,14,17,20,23,26,31-nonaoxo-18- | |
| | phenethyl-1,4,7,10,13,16,19,22,25-nonaazacyclohentriacontan-12-yl)methyl)- | |
| | 1H-indol-5-yl acetate | |
| A4 | 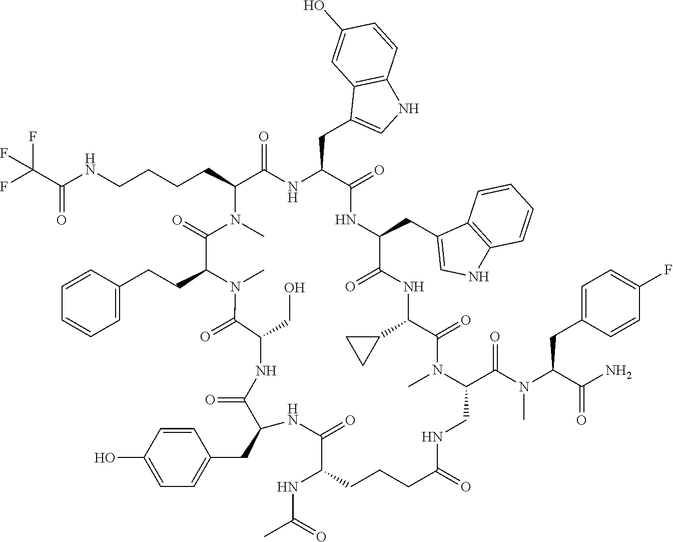 | Method AP-1 t_{R} = 5.21 min [M + H]⁺ = 1612.7 |
| | (3S,6S,9S,12S,15S,18S,21S,24S,27S)-9-((1H-indol-3-yl)methyl)-27-acetamido- | |
| | N-((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,31-nonaoxo-18-phenethyl-15-(4- | |
| | (2,2,2-trifluoroacetamido)butyl)-1,4,7,10,13,16,19,22,25- | |
| | nonaazacyclohentriacontane-3-carboxamide | |
| A5 | 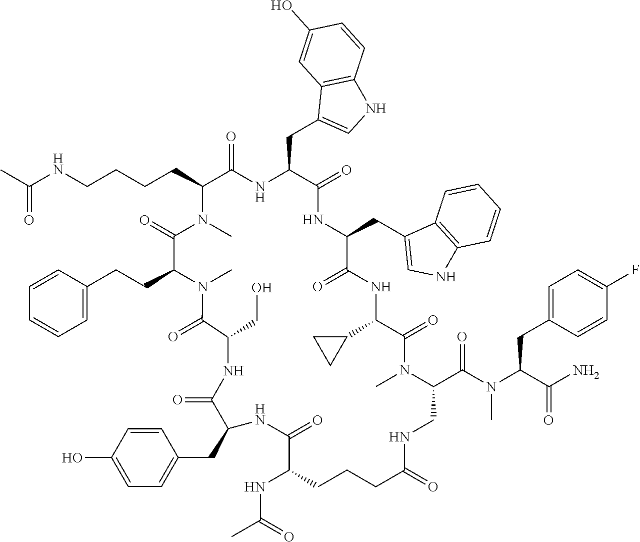 | Method AP-1 t_{R} = 4.57 min [M + H]⁺ = 1558.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 6-cyclopropyl-12-((5-hydroxy-1H-indol-3-yl)methyl)-24-(4-hydroxybenzyl)- | |
| | 21-(hydroxymethyl)-N,4,16,19-tetramethyl-5,8,11,14,17,20,23,26,31-nonaoxo- | |
| | 18-phenethyl-1,4,7,10,13,16,19,22,25-nonaazacyclohentriacontane-3- | |
| | carboxamide | |
| A6 | 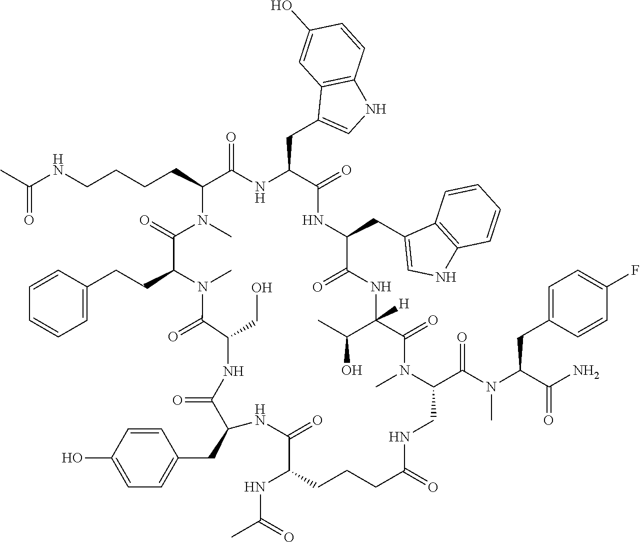 | Method AP-1 t_{R} = 4.40 min [M + 2H]²⁺ = 781.9 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 12-((5-hydroxy-1H-indol-3-yl)methyl)-24-(4-hydroxybenzyl)-6-((S)-1- | |
| | hydroxyethyl)-21-(hydroxymethyl)-N,4,16,19-tetramethyl- | |
| | 5,8,11,14,17,20,23,26,31-nonaoxo-18-phenethyl-1,4,7,10,13,16,19,22,25- | |
| | nonaazacyclohentriacontane-3-carboxamide | |
| A7 | 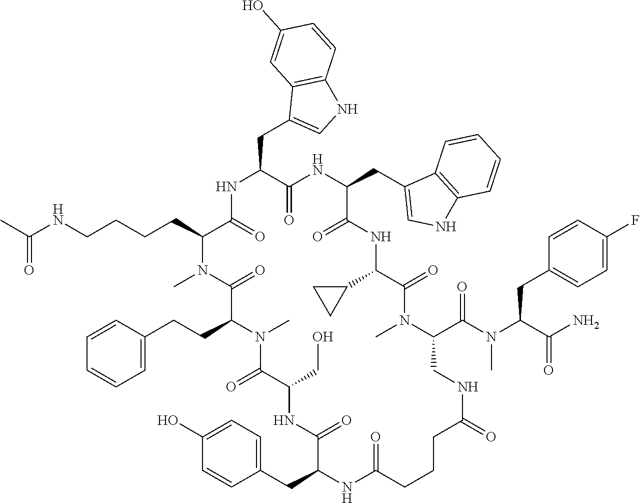 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(N)=O)CNC(=O)CCCC(=O)N[C@@H](Cc2ccc(O)cc2)C(=O)N[C@@H](CO)C(=O)N(C)[C@@H](CCc2ccccc2)C(=O)N1C] | Method AP-1 t_{R} = 4.64 min [M + H]⁺ = 1487.7 |
| | (3S,6S,9S,12S,15S,18S,21S,24S)-9-((1H-indol-3-yl)methyl)-15-(4- | |
| | acetamidobutyl)-N-((S)-1-amino-3-(4-fluorophenyl)-1-oxopropan-2-yl)-6- | |
| | cyclopropyl-12-((5-hydroxy-1H-indol-3-yl)methyl)-24-(4-hydroxybenzyl)-21- | |
| | (hydroxymethyl)-N,4,16,19-tetramethyl-5,8,11,14,17,20,23,26,30-nonaoxo-18- | |
| | phenethyl-1,4,7,10,13,16,19,22,25-nonaazacyclotriacontane-3-carboxamide | |
| A8 | 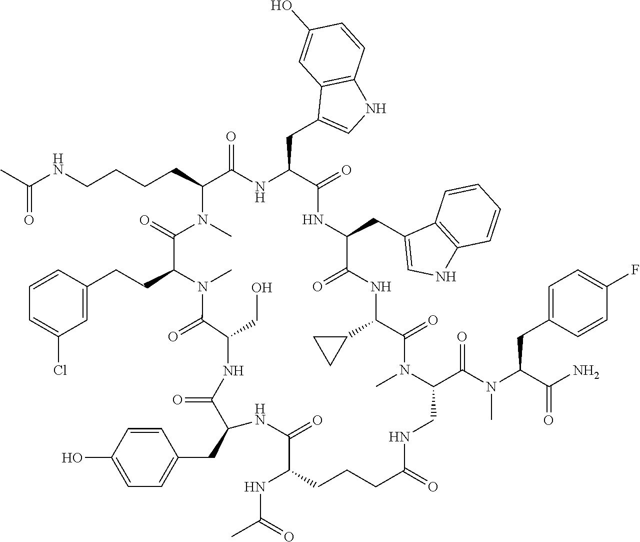 | Method AP-1 t_{R} = 4.88 min [M + H]⁺ = 1592.7 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 18-(3-chlorophenethyl)-6-cyclopropyl-12-((5-hydroxy-1H-indol-3-yl)methyl)- | |
| | 24-(4-hydroxybenzyl)-21-(hydroxymethyl)-N,4,16,19-tetramethyl- | |
| | 5,8,11,14,17,20,23,26,31-nonaoxo-1,4,7,10,13,16,19,22,25- | |
| | nonaazacyclohentriacontane-3-carboxamide | |
| A9 (SEQ ID NO: 2) | 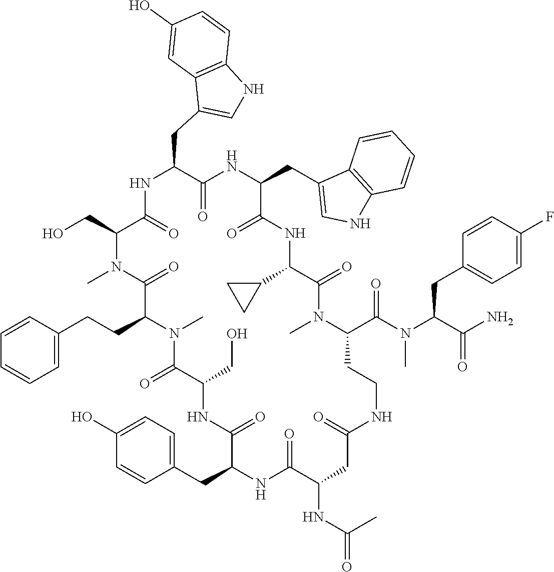 [SMILES: CC(=O)N[C@H]1CC(=O)NCC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.56 min [M + H]⁺ = 1461.7 |
| | (2S,5S,8S,11S,14S,17S,20S,23S,29S)-17-((1H-indol-3-yl)methyl)-29- | |
| | acetamido-N-((S)-1-amino-3-(4-fluorophenyl)-1-oxopropan-2-yl)-20- | |
| | cyclopropyl-14-((5-hydroxy-1H-indol-3-yl)methyl)-2-(4-hydroxybenzyl)-5,11- | |
| | bis(hydroxymethyl)-N,7,10,22-tetramethyl-3,6,9,12,15,18,21,27,30-nonaoxo-8- | |
| | phenethyl-1,4,7,10,13,16,19,22,26-nonaazacyclotriacontane-23-carboxamide | |
| A10 (SEQ ID NO: 3) | 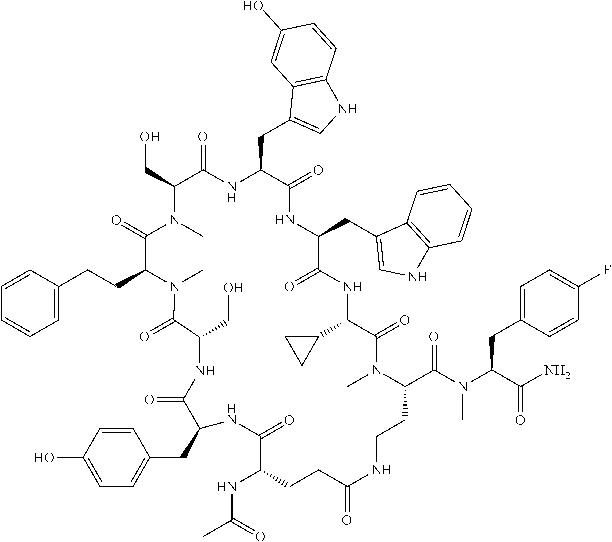 | Method AP-1 t_{R} = 4.48 min [M + H]⁺ = 1475.7 |
| | (2S,5S,8S,11S,14S,17S,20S,23S,30S)-17-((1H-indol-3-yl)methyl)-30- | |
| | acetamido-N-((S)-1-amino-3-(4-fluorophenyl)-1-oxopropan-2-yl)-20- | |
| | cyclopropyl-14-((5-hydroxy-1H-indol-3-yl)methyl)-2-(4-hydroxybenzyl)-5,11- | |
| | bis(hydroxymethyl)-N,7,10,22-tetramethyl-3,6,9,12,15,18,21,27,31-nonaoxo-8- | |
| | phenethyl-1,4,7,10,13,16,19,22,26-nonaazacyclohentriacontane-23- | |
| | carboxamide | |
| A11 | 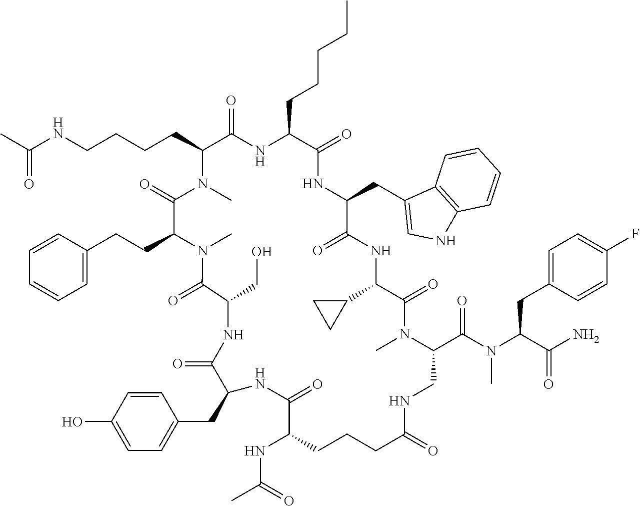 | Method AP-1 t_{R} = 5.26 min [M + H]⁺ = 1483.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 6-cyclopropyl-24-(4-hydroxybenzyl)-21-(hydroxymethyl)-N,4,16,19- | |
| | tetramethyl-5,8,11,14,17,20,23,26,31-nonaoxo-12-pentyl-18-phenethyl- | |
| | 1,4,7,10,13,16,19,22,25-nonaazacyclohentriacontane-3-carboxamide | |
| A12 (SEQ ID NO: 4) | 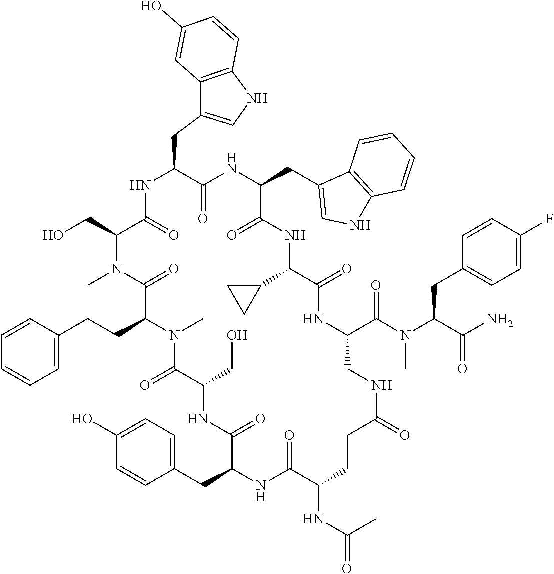 [SMILES: CC(=O)N[C@H]1CCC(=O)NC[C@@H](C(=O)N(C)[C@@H](Cc2ccc(F)cc2)C(N)=O)NC(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.41 min [M + H]⁺ = 1447.6 |
| | (3S,6S,9S,12S,15S,18S,21S,24S,27S)-9-((1H-indol-3-yl)methyl)-27-acetamido- | |
| | N-((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-15,21- | |
| | bis(hydroxymethyl)-N,16,19-trimethyl-5,8,11,14,17,20,23,26,30-nonaoxo-18- | |
| | phenethyl-1,4,7,10,13,16,19,22,25-nonaazacyclotriacontane-3-carboxamide | |
| A13 | 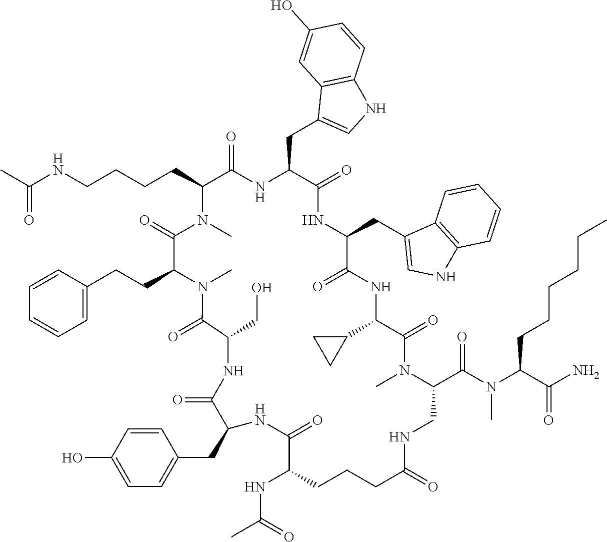 | Method AP-1 t_{R} = 5.07 min [M + H]⁺ = 1534.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-1-oxooctan-2-yl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,31-nonaoxo-18-phenethyl- | |
| | 1,4,7,10,13,16,19,22,25-nonaazacyclohentriacontane-3-carboxamide | |
| A14 | 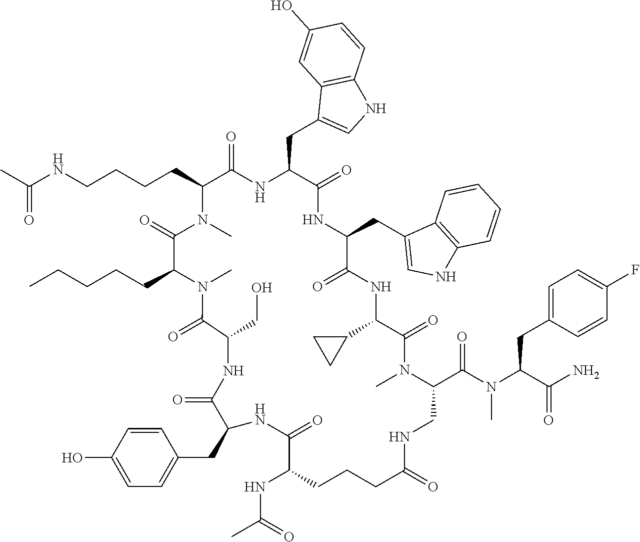 | Method AP-1 t_{R} = 4.69 min [M + H]⁺ = 1524.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 6-cyclopropyl-12-((5-hydroxy-1H-indol-3-yl)methyl)-24-(4-hydroxybenzyl)- | |
| | 21-(hydroxymethyl)-N,4,16,19-tetramethyl-5,8,11,14,17,20,23,26,31-nonaoxo- | |
| | 18-pentyl-1,4,7,10,13,16,19,22,25-nonaazacyclohentriacontane-3-carboxamide | |
| A15 | 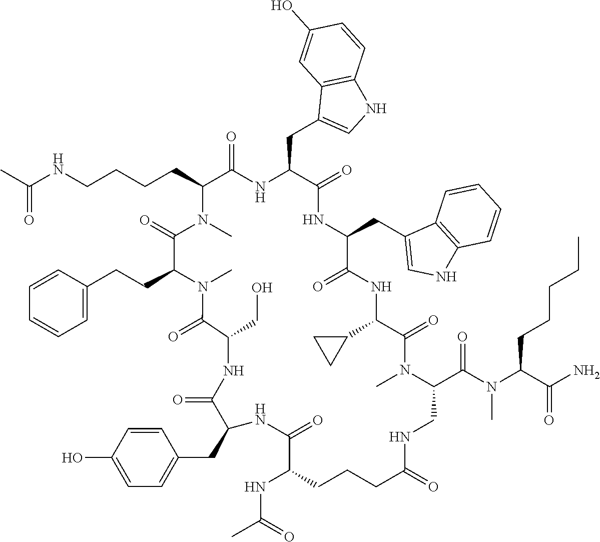 | Method AP-1 t_{R} = 4.84 min [M + H]⁺ = 1520.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-1-oxoheptan-2-yl)-6-cyclopropyl-12- | |
| | ((5-hydroxy-1H-indol-3-yl)methyl)-24-(4-hydroxybenzyl)-21- | |
| | (hydroxymethyl)-N,4,16,19-tetramethyl-5,8,11,14,17,20,23,26,31-nonaoxo-18- | |
| | phenethyl-1,4,7,10,13,16,19,22,25-nonaazacyclohentriacontane-3-carboxamide | |
| A16 (SEQ ID NO: 5) | 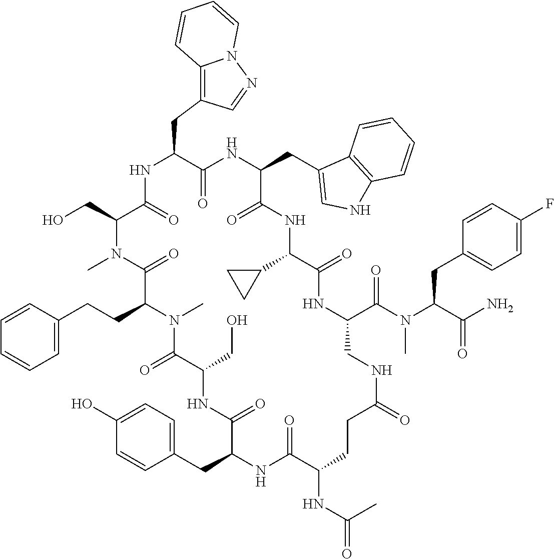 | Method AP-1 t_{R} = 4.62 min [M + H]⁺ = 1446.7 |
| | (3S,6S,9S,12S,15S,18S,21S,24S,27S)-9-((1H-indol-3-yl)methyl)-27-acetamido- | |
| | N-((S)-1-amino-3-(4-fluorophenyl)-1-oxopropan-2-yl)-6-cyclopropyl-24-(4- | |
| | hydroxybenzyl)-15,21-bis(hydroxymethyl)-N,4,16,19-tetramethyl- | |
| | 5,8,11,14,17,20,23,26,30-nonaoxo-18-phenethyl-12-(pyrazolo[1,5-a]pyridin-3- | |
| | ylmethyl)-1,4,7,10,13,16,19,22,25-nonaazacyclotriacontane-3-carboxamide | |
| | TFA salt | |
| A17 | 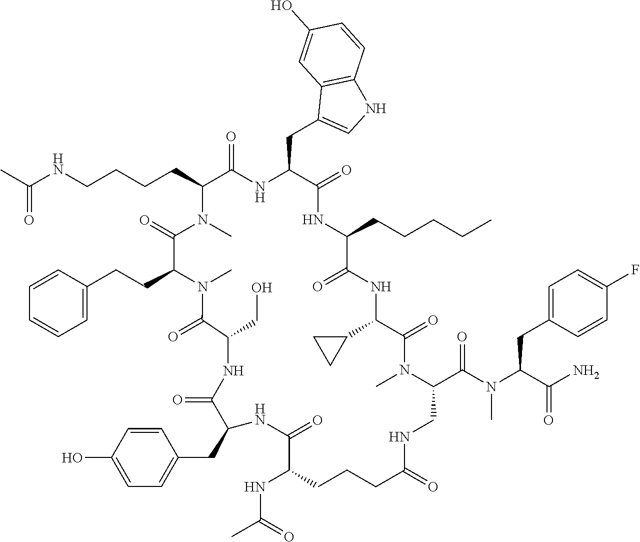 | Method AP-1 t_{R} = 4.80 min [M + H]⁺ = 1499.8 |
| | (3S,6S,9S,12S,15S,18S,21S,24S,27S)-27-acetamido-15-(4-acetamidobutyl)-N- | |
| | ((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,31-nonaoxo-9-pentyl-18- | |
| | phenethyl-1,4,7,10,13,16,19,22,25-nonaazacyclohentriacontane-3-carboxamide | |
| A18 | 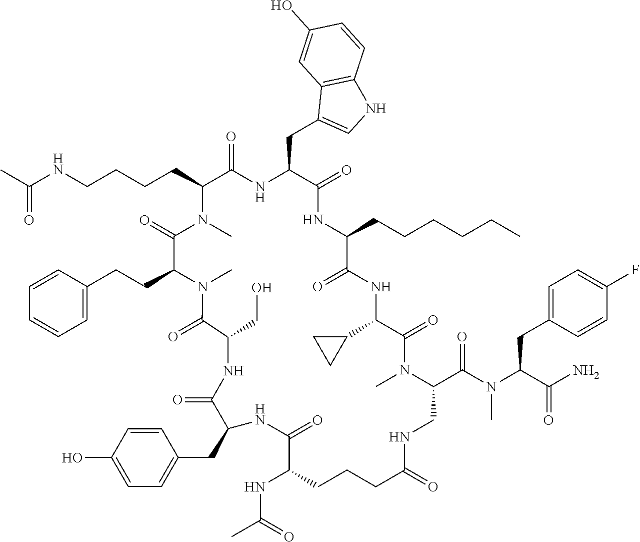 | Method AP-1 t_{R} = 5.03 min [M + H]⁺ = 1513.8 |
| | (3S,6S,9S,12S,15S,18S,21S,24S,27S)-27-acetamido-15-(4-acetamidobutyl)-N- | |
| | ((S)-1-amino-3-(4-fluorophenyl)-1-oxopropan-2-yl)-6-cyclopropyl-9-hexyl-12- | |
| | ((5-hydroxy-1H-indol-3-yl)methyl)-24-(4-hydroxybenzyl)-21- | |
| | (hydroxymethyl)-N,4,16,19-tetramethyl-5,8,11,14,17,20,23,26,31-nonaoxo-18- | |
| | phenethyl-1,4,7,10,13,16,19,22,25-nonaazacyclohentriacontane-3-carboxamide | |
| A19 | 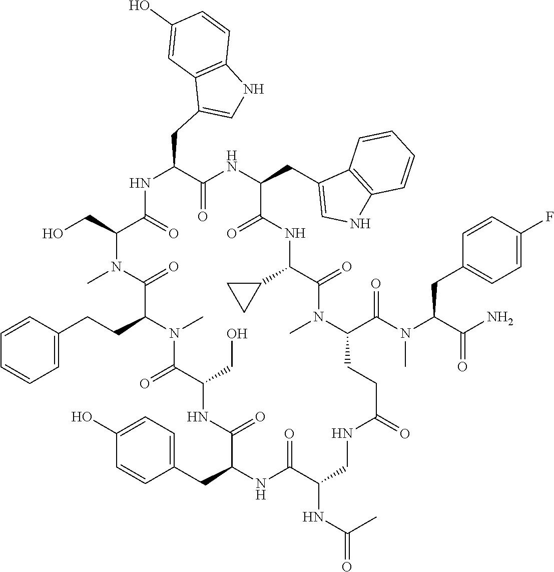 [SMILES: CC(=O)N[C@H]1CNC(=O)CC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.47 min [M + H]⁺ = 1461.7 |
| | (3S,6S,9S,12S,15S,18S,21S,24S,30S)-6-((1H-indol-3-yl)methyl)-24-acetamido- | |
| | N-((S)-1-amino-3-(4-fluorophenyl)-1-oxopropan-2-yl)-3-cyclopropyl-9-((5- | |
| | hydroxy-1H-indol-3-yl)methyl)-21-(4-hydroxybenzyl)-12,18- | |
| | bis(hydroxymethyl)-N,1,13,16-tetramethyl-2,5,8,11,14,17,20,23,27-nonaoxo- | |
| | 15-phenethyl-1,4,7,10,13,16,19,22,26-nonaazacyclotriacontane-30- | |
| | carboxamide | |
| A20 | 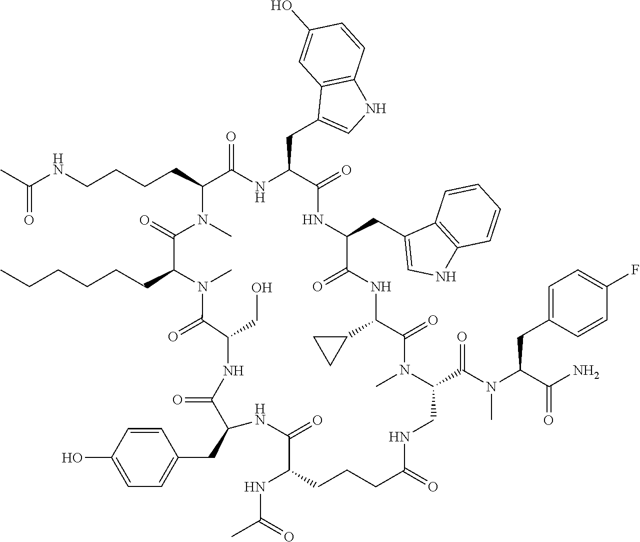 | Method AP-1 t_{R} = 4.90 min [M + H]⁺ = 1538.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-yl)- | |
| | 6-cyclopropyl-18-hexyl-12-((5-hydroxy-1H-indol-3-yl)methyl)-24-(4- | |
| | hydroxybenzyl)-21-(hydroxymethyl)-N,4,16,19-tetramethyl- | |
| | 5,8,11,14,17,20,23,26,31-nonaoxo-1,4,7,10,13,16,19,22,25- | |
| | nonaazacyclohentriacontane-3-carboxamide | |
| A21 | 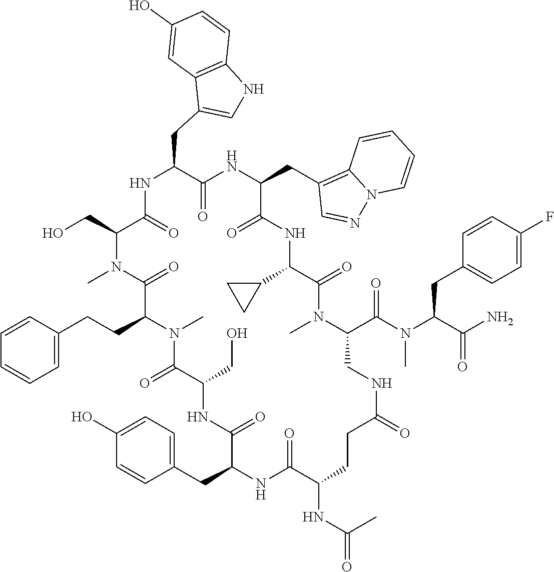 [SMILES: CC(=O)N[C@H]1CCC(=O)NC[C@@H](C(=O)N(C)[C@@H](Cc2ccc(F)cc2)C(N)=O)N(C)C(=O)[C@H](C2CC2)NC(=O)[C@H](Cc2cnn3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.30 min [M + H]⁺ = 1462.6 |
| | (3S,6S,9S,12S,15S,18S,21S,24S,27S)-27-acetamido-N-((S)-1-amino-3-(4- | |
| | fluorophenyl)-1-oxopropan-2-yl)-6-cyclopropyl-12-((5-hydroxy-1H-indol-3- | |
| | yl)methyl)-24-(4-hydroxybenzyl)-15,21-bis(hydroxymethyl)-N,4,16,19- | |
| | tetramethyl-5,8,11,14,17,20,23,26,30-nonaoxo-18-phenethyl-9-(pyrazolo[1,5- | |
| | a]pyridin-3-ylmethyl)-1,4,7,10,13,16,19,22,25-nonaazacyclotriacontane-3- | |
| | carboxamide TFA salt | |
| A22 | 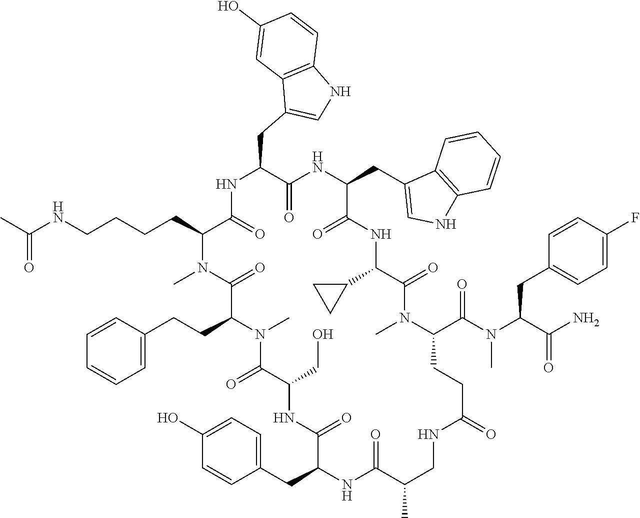 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(N)=O)CCC(=O)NC[C@H](C)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 4.73 min [M + H]⁺ = 1501.7 |
| | (3S,6S,9S,12S,15S,18S,21S,24S,30S)-6-((1H-indol-3-yl)methyl)-12-(4- | |
| | acetamidobutyl)-N-((S)-1-amino-3-(4-fluorophenyl)-1-oxopropan-2-yl)-3- | |
| | cyclopropyl-9-((5-hydroxy-1H-indol-3-yl)methyl)-21-(4-hydroxybenzyl)-18- | |
| | (hydroxymethyl)-N,1,13,16,24-pentamethyl-2,5,8,11,14,17,20,23,27-nonaoxo- | |
| | 15-phenethyl-1,4,7,10,13,16,19,22,26-nonaazacyclotriacontane-30- | |
| | carboxamide | |
| A23 | 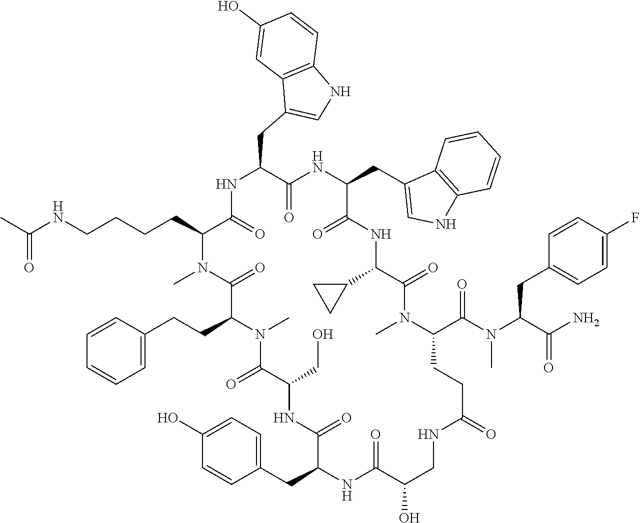 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(N)=O)CCC(=O)NC[C@H](O)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 4.59 min [M + H]⁺ = 1503.7 |
| | (3S,6S,9S,12S,15S,18S,21S,24S,30S)-6-((1H-indol-3-yl)methyl)-12-(4- | |
| | acetamidobutyl)-N-((S)-1-amino-3-(4-fluorophenyl)-1-oxopropan-2-yl)-3- | |
| | cyclopropyl-24-hydroxy-9-((5-hydroxy-1H-indol-3-yl)methyl)-21-(4- | |
| | hydroxybenzyl)-18-(hydroxymethyl)-N,1,13,16-tetramethyl- | |
| | 2,5,8,11,14,17,20,23,27-nonaoxo-15-phenethyl-1,4,7,10,13,16,19,22,26- | |
| | nonaazacyclotriacontane-30-carboxamide | |
| A24 | 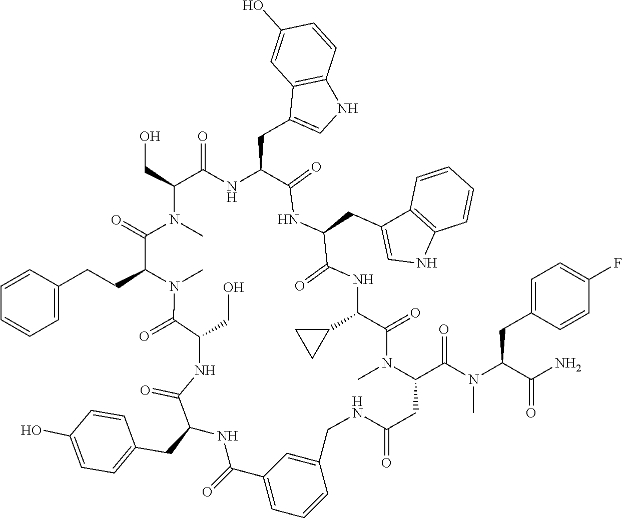 | Method AP-1 t_{R} = 4.83 min [M + H]⁺ = 1452.6 |

(4S,7S,10S,13S,16S,19S,22S,25S)-19-((1H-indol-3-yl)methyl)-N-((S)-1-amino-
3-(4-fluorophenyl)-1-oxopropan-2-yl)-22-cyclopropyl-16-((5-hydroxy-1H-
indol-3-yl)methyl)-4-(4-hydroxybenzyl)-7,13-bis(hydroxymethyl)-N,9,12,24-
tetramethyl-2,5,8,11,14,17,20,23,27-nonaoxo-10-phenethyl-
3,6,9,12,15,18,21,24,28-nonaaza-1(1,3)-benzenacyclononacosaphane-25-
carboxamide

### 2.1.2 Synthesis of (3S,6S,9S,12S,15S,18S,21S,24S,27S)-9-((1H-indol-3-yl)methyl)-27-acetamido-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-15,21-bis(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamide (Example A25 (A25-2 disclosed as SEQ ID NO: 110 and A25 disclosed as SEQ ID NO: 6))

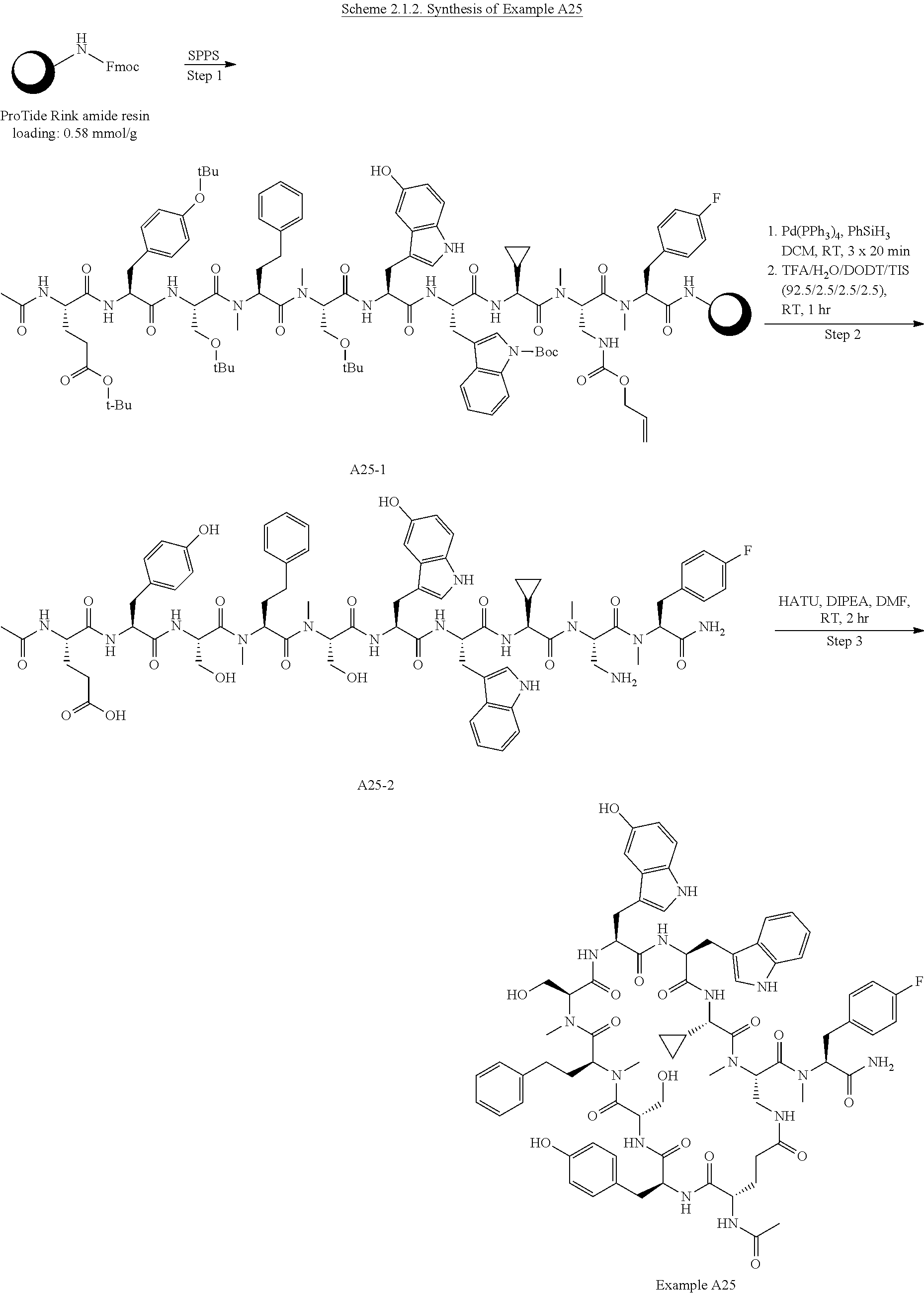

### Step 1. A25-1

The assembly of the linear peptide was done on the CEM Liberty Blue Synthesizer using commercial Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×4 min at 90° C.
Method B Fmoc removal: 2×1 min at 90° C./Coupling: 2× 4 min at 90° C.

After the assembly of the linear peptide the resin was washed with DMF (5×) and DCM (5×). The amino acids and coupling methods are summarized in Table 2.3.

TABLE 2.3

| Synthesis | Amino acid | | |
|---|---|---|---|
| cycle | residue^{a} | Amino acid | Method |
| 1 | A¹⁰ | Fmoc-L-N-Me-Phe(4F)-OH | A |
| 2 | A⁹ | Fmoc-L-N-Me-Dap(Alloc)- | B |
| | | OH | |
| 3 | A⁸ | Fmoc-L-CyclopropylGly-OH | B |
| 4 | A⁷ | Fmoc-L-Trp(Boc)-OH | A |
| 5 | A⁶ | Fmoc-L-Trp(5-OH)-OH | A |
| 6 | A⁵ | Fmoc-L-N-Me-Ser(tBu)-OH | A |
| 7 | A⁴ | Fmoc-L-N-Me-Homo-Phe- | B |
| | | OH | |
| 8 | A³ | Fmoc-L-Ser(tBu)-OH | A |
| 9 | A² | Fmoc-L-Tyr(tBu)-OH | A |
| 10 | A¹ | Fmoc-L-Glu(OtBu)-OH | A |
| 11 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

### Step 2. A25-2

Step 2-1: The resin from Step 1 (calculated with 100 μmol) was suspended with DCM (2 mL) and treated with phenylsilane (216 mg, 249 μL, 20 eq., 2 mmol). The suspension was shaken at RT for 10 min while purged with argon. A clear solution of Pd(PPh₃)₄ (11.56 mg, 0.1 eq., 10.0 μmol) in DCM (1 mL) was added at RT. The reaction was agitated with argon for 20 min. The resin was drained, washed with DMA (3×) and DCM (5×), and the procedure was repeated twice. The resin was drained and washed with DMA (3×) and DCM (5×).

Step 2-2: To the resin from Step 2-1 was added TFA/H₂O/DODT/TIS (92.5/2.5/2.5/2.5) (4 mL) and the resin was shaken at RT for 1 hr. The crude peptide was precipitated with cold diethyl ether/heptane (1/1) (45 mL) giving a precipitate. The suspension was centrifuged, and the solvent was decanted. The product was washed with another portion of cold diethyl ether/heptane (1/1) (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A25-2 (41 mg, 26 μmol, 25% yield, TFA salt) as a white solid. LCMS Method AP-1, t_{R}=3.63 min, [M+H]⁺=1479.6.

### Step 3. Example A25

A25-2 (41 mg, 1 eq., 26 μmol, TFA salt) was dissolved in DMF (4 mL). Then HATU (14.7 mg, 1.5 eq., 39 μmol), followed by DIPEA (9.98 mg, 13 μL, 3 eq., 77 μmol) were added. The reaction mixture was stirred at RT for 2 hr. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A25 (8 mg, 5.2 μmol, 20% yield) as a white solid. LCMS Method AP-1, t_{R}=4.53 min, [M+H]⁺=1461.7.

The compounds in Table 2.4 were synthesized in analogy to Example A25.

TABLE 2.4
Examples synthesized in analogy to Example A25 (Scheme 2.1.2).

| Ex. No. | Structure/Example Name | LCMS |
|---|---|---|
| A26 (SEQ ID NO: 7) | 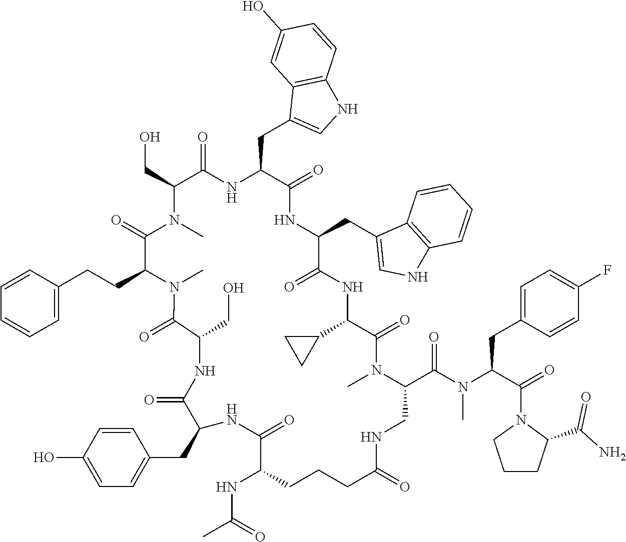 | Method AP-1 t_{R} = 4.64 min [M + H]⁺ = 1572.7 |
| | (3S,6S,9S,12S,15S,18S,21S,24S,27S)-9-((1H-indol-3-yl)methyl)-27- | |
| | acetamido-N-((S)-1-((S)-2-carbamoylpyrrolidin-1-yl)-3-(4-fluorophenyl)-1- | |
| | oxopropan-2-yl)-6-cyclopropyl-12-((5-hydroxy-1H-indol-3-yl)methyl)-24-(4- | |
| | hydroxybenzyl)-15,21-bis(hydroxymethyl)-N,4,16,19-tetramethyl- | |
| | 5,8,11,14,17,20,23,26,31-nonaoxo-18-phenethyl-1,4,7,10,13,16,19,22,25- | |
| | nonaazacyclohentriacontane-3-carboxamide | |
| A27 | 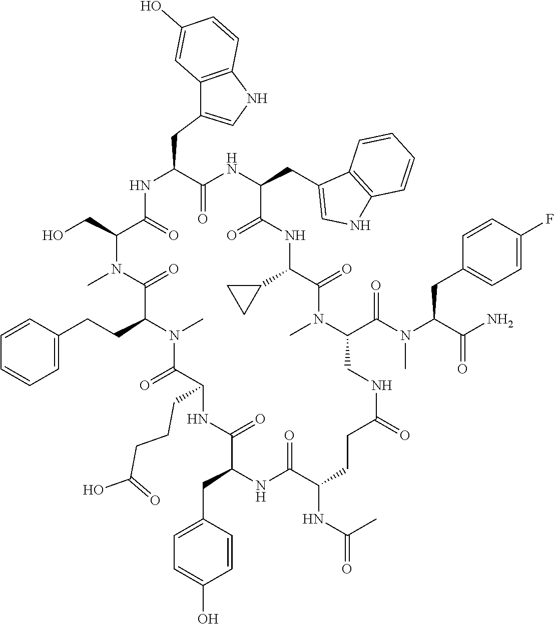 [SMILES: CC(=O)N[C@H]1CCC(=O)NC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CCCC(=O)O)NC(=O)[C@H](Cc2ccc(O)cc2)NC1=O] | Method AP-1 t_{R} = 4.61 min [M + H]⁺ = 1517.7 |
| | 4-((2S,5S,8S,11S,14S,17S,20S,23S,29S)-17-((1H-indol-3-yl)methyl)-29- | |
| | acetamido-23-(((S)-1-amino-3-(4-fluorophenyl)-1-oxopropan-2- | |
| | yl)(methyl)carbamoyl)-20-cyclopropyl-14-((5-hydroxy-1H-indol-3- | |
| | yl)methyl)-2-(4-hydroxybenzyl)-11-(hydroxymethyl)-7,10,22-trimethyl- | |
| | 3,6,9,12,15,18,21,26,30-nonaoxo-8-phenethyl-1,4,7,10,13,16,19,22,25- | |
| | nonaazacyclotriacontan-5-yl)butanoic acid | |
| A28 (SEQ ID NO: 8) | 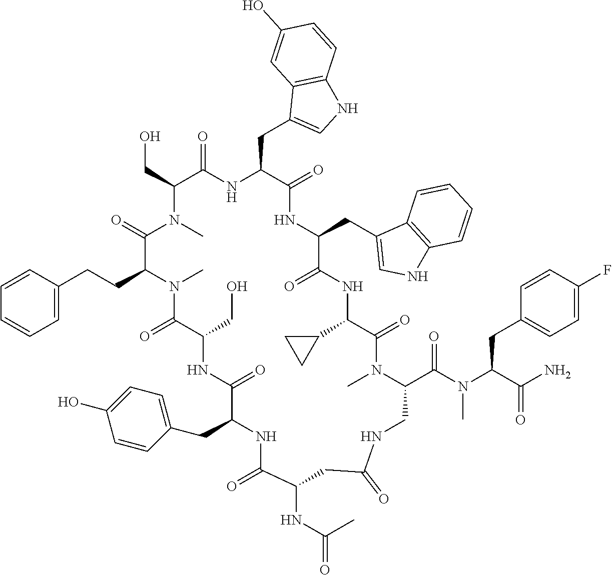 | Method AP-1 t_{R} = 4.55 min [M + H]⁺ = 1447.6 |
| | (3S,6S,9S,12S,15S,18S,21S,24S,27S)-9-((1H-indol-3-yl)methyl)-27- | |
| | acetamido-N-((S)-1-amino-3-(4-fluorophenyl)-1-oxopropan-2-yl)-6- | |
| | cyclopropyl-12-((5-hydroxy-1H-indol-3-yl)methyl)-24-(4-hydroxybenzyl)- | |
| | 15,21-bis(hydroxymethyl)-N,4,16,19-tetramethyl-5,8,11,14,17,20,23,26,29- | |
| | nonaoxo-18-phenethyl-1,4,7,10,13,16,19,22,25-nonaazacyclononacosane-3- | |
| | carboxamide | |
| A29 | 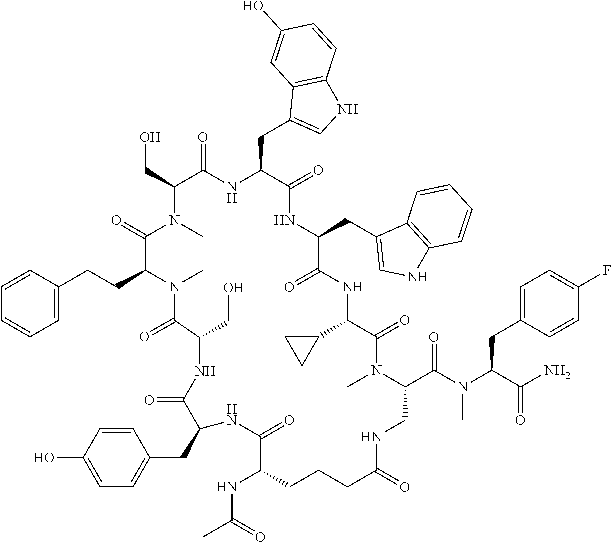 | Method AP-1 t_{R} = 4.64 min [M + 2H]²⁺ = 738.3 |
| | (3S,6S,9S,12S,15S,18S,21S,24S,27S)-9-((1H-indol-3-yl)methyl)-27- | |
| | acetamido-N-((S)-1-amino-3-(4-fluorophenyl)-1-oxopropan-2-yl)-6- | |
| | cyclopropyl-12-((5-hydroxy-1H-indol-3-yl)methyl)-24-(4-hydroxybenzyl)- | |
| | 15,21-bis(hydroxymethyl)-N,4,16,19-tetramethyl-5,8,11,14,17,20,23,26,31- | |
| | nonaoxo-18-phenethyl-1,4,7,10,13,16,19,22,25-nonaazacyclohentriacontane- | |
| | 3-carboxamide | |
| A30 (SEQ ID NO: 9) | 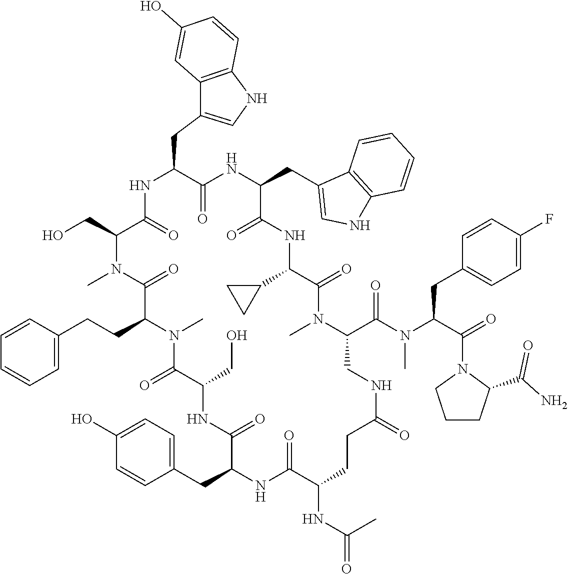 [SMILES: CC(=O)N[C@H]1CCC(=O)NC[C@@H](C(=O)N(C)[C@@H](Cc2ccc(F)cc2)C(=O)N2CCC[C@H]2C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.59 min [M + H]⁺ = 1558.6 |

(3S,6S,9S,12S,15S,18S,21S,24S,27S)-9-((1H-indol-3-yl)methyl)-27-
acetamido-N-((S)-1-((S)-2-carbamoylpyrrolidin-1-yl)-3-(4-fluorophenyl)-1-
oxopropan-2-yl)-6-cyclopropyl-12-((5-hydroxy-1H-indol-3-yl)methyl)-24-(4-
hydroxybenzyl)-15,21-bis(hydroxymethyl)-N,4,16,19-tetramethyl-
5,8,11,14,17,20,23,26,30-nonaoxo-18-phenethyl-1,4,7,10,13,16,19,22,25-
nonaazacyclotriacontane-3-carboxamide

### 2.1.3 Synthesis of (6S,9S,12S,15S,18S,21S,24S,27S,30S)-12-((1H-indol-3-yl)methyl)-30-amino-N—((S)-1-amino-3-(4-fluorophenyl)-1-oxopropan-2-yl)-9-cyclopropyl-15-((5-hydroxy-1H-indol-3-yl)methyl)-27-(4-hydroxybenzyl)-18,24-bis(hydroxymethyl)-N,7,19,22-tetramethyl-3,8,11,14,17,20,23,26,29-nonaoxo-21-phenethyl-4,7,10,13,16,19,22,25,28-nonaaza-1 (1,3)-benzenacyclohentriacontaphane-6-carboxamide TFA salt (Example A31)

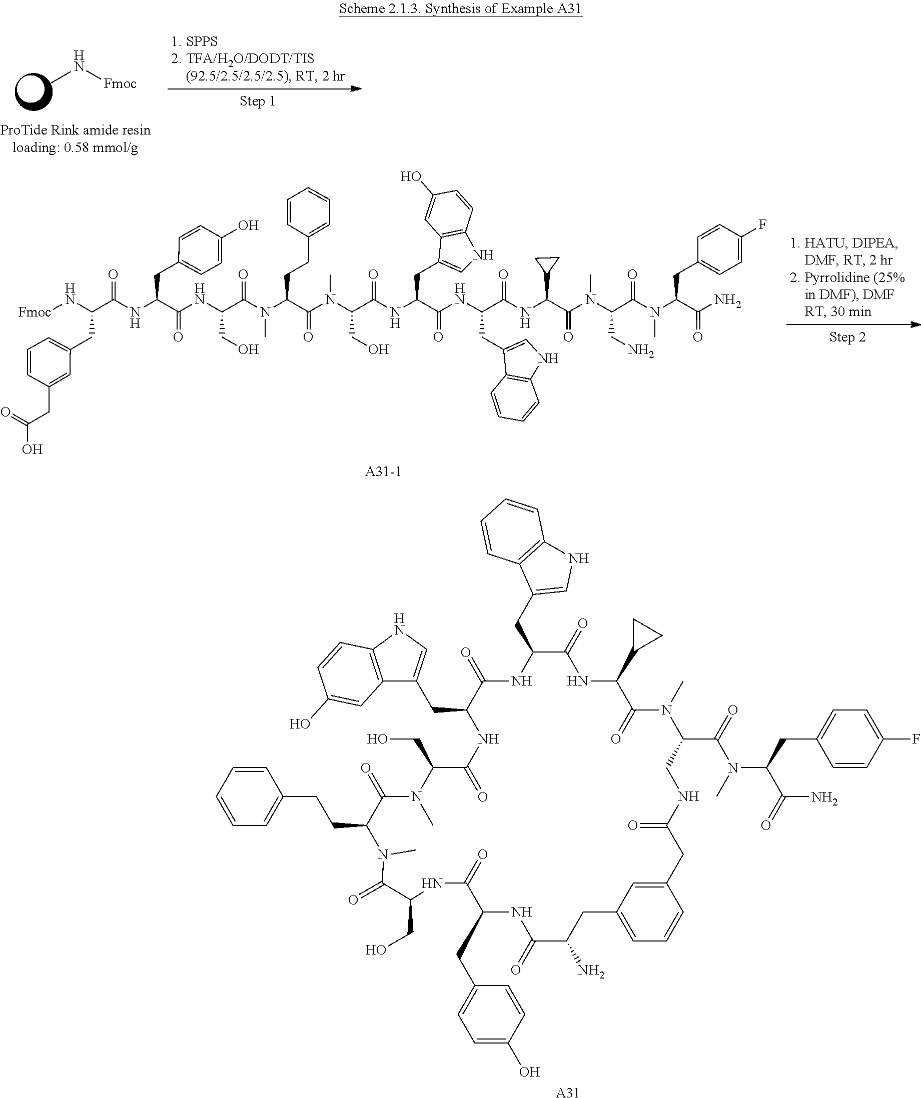

### Step 1. A31-1

Step 1-1: The assembly of the linear peptide was done on the CEM Liberty Prime Synthesizer using commercial Rink amide resin R1 (loading 0.58 mmol/g, 172 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.
Method C Fmoc removal: 2×10 min at RT/Coupling: 2×4 min at 105° C.
Method D Fmoc removal: 2×10 min at RT/Coupling: 1×2 min at 105° C.

After the assembly of the linear peptide, the resin was washed with DMF (5×) and DCM (5×). The amino acids and coupling methods are summarized in Table 2.5.

TABLE 2.5

| Synthesis | Amino acid | | |
|---|---|---|---|
| cycle | residue^{a} | Amino acid | Method |
| 1 | A¹⁰ | Fmoc-L-N-Me-Phe(4F)-OH | A |
| 2 | A⁹ | Fmoc-L-N-Me-Dap(Boc)- | B |
| | | OH | |
| 3 | A⁸ | Fmoc-L-CyclopropylGly- | B |
| | | OH | |
| 4 | A⁷ | Fmoc-L-Trp(Boc)-OH | A |
| 5 | A⁶ | Fmoc-L-Trp(5-OH)-OH | A |
| 6 | A⁵ | Fmoc-L-N-Me-Ser(tBu)- | C |
| | | OH | |
| 7 | A⁴ | Fmoc-L-N-Me-Homo-Phe- | B |
| | | OH | |
| 8 | A³ | Fmoc-L-Ser(tBu)-OH | B |
| 9 | A² | Fmoc-L-Tyr(tBu)-OH | D |
| 10 | A¹ | Fmoc-L-F3AA(OtBu)-OH | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (5 mL) and the resin was shaken at RT for 2 hr. The crude peptide was precipitated with cold diethyl ether (40 mL). The suspension was centrifuged, and the solvent was decanted. The product was washed with another portion of cold diethyl ether (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A31-1 (43.9 mg, 25.3 μmol, 30% yield, TFA salt) as a white solid. LCMS Method AP-1, t_{R}=4.98 min, [M+H]⁺=1736.8.

### Step 2. Example A31

Step 2-1: A31-1 (24 mg, 1 eq., 19 μmol TFA salt) was dissolved in DMF (3 mL). Then HATU (7.4 mg, 1.5 eq., 19 μmol), followed by DIPEA (5 mg, 6.8 μL, 3 eq., 39 μmol) were added. The reaction mixture was stirred at RT for 2 hr.

Step 2-2: The reaction mixture from Step 2-1 was directly treated with pyrrolidine (25% in DMF) (92 mg, 110 μL, 25 eq., 320 μmol) and shaken at RT for 30 min, then quenched with ACN/water (1/1). The quenched reaction mixture was directly purified by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A31 (5.1 mg, 3 μmol, 23% yield, TFA salt) as a white solid. LCMS Method AP-1, t_{R}=3.93 min, [M+H]⁺=1495.7.

The compounds in Table 2.6 were synthesized in analogy to Example A31.

TABLE 2.6
Examples synthesized in analogy to Example A31 (Scheme 2.1.3).

| Ex. No. | Structure/Example Name | LCMS |
|---|---|---|
| A32 (SEQ ID NO: 10) | 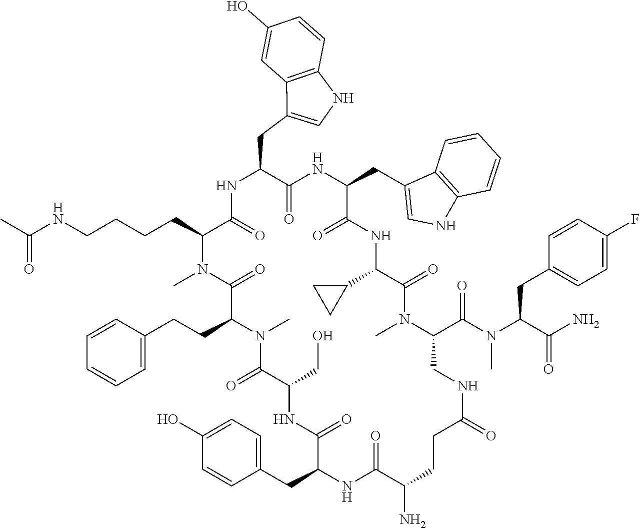 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(N)=O)CNC(=O)CC[C@H](N)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 3.94 min [M + H]⁺ = 1502.7 |
| | (3S,6S,9S,12S,15S,18S,21S,24S,27S)-9-((1H-indol-3-yl)methyl)-15-(4- | |
| | acetamidobutyl)-27-amino-N-((S)-1-amino-3-(4-fluorophenyl)-1-oxopropan-2- | |
| | yl)-6-cyclopropyl-12-((5-hydroxy-1H-indol-3-yl)methyl)-24-(4- | |
| | hydroxybenzyl)-21-(hydroxymethyl)-N,4,16,19-tetramethyl- | |
| | 5,8,11,14,17,20,23,26,30-nonaoxo-18-phenethyl-1,4,7,10,13,16,19,22,25- | |
| | nonaazacyclotriacontane-3-carboxamide TFA salt | |
| A33 (SEQ ID NO: 11) | 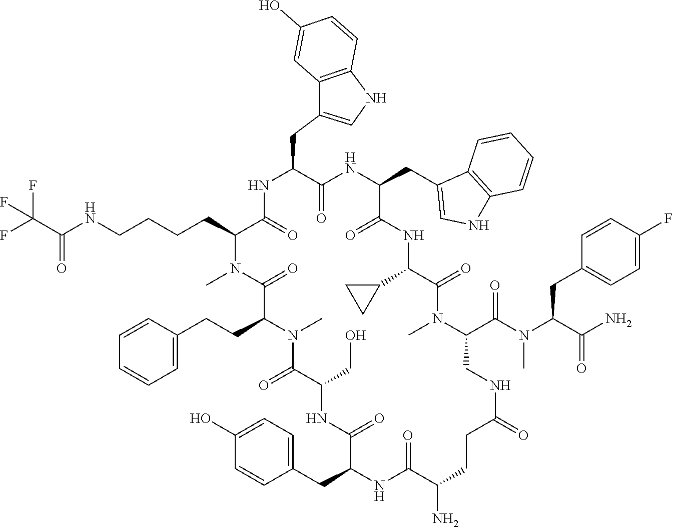 [SMILES: CN1C(=O)[C@H](CO)NC(=O)[C@H](Cc2ccc(O)cc2)NC(=O)[C@@H](N)CCC(=O)NC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(=O)C(F)(F)F)N(C)C(=O)[C@@H]1CCc1ccccc1] | Method AP-1 t_{R} = 4.32 min [M + H]⁺ = 1556.7 |
| | (3S,6S,9S,12S,15S,18S,21S,24S,27S)-9-((1H-indol-3-yl)methyl)-27-amino-N- | |
| | ((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,30-nonaoxo-18-phenethyl-15-(4- | |
| | (2,2,2-trifluoroacetamido)butyl)-1,4,7,10,13,16,19,22,25- | |
| | nonaazacyclotriacontane-3-carboxamide TFA salt | |
| A34 | 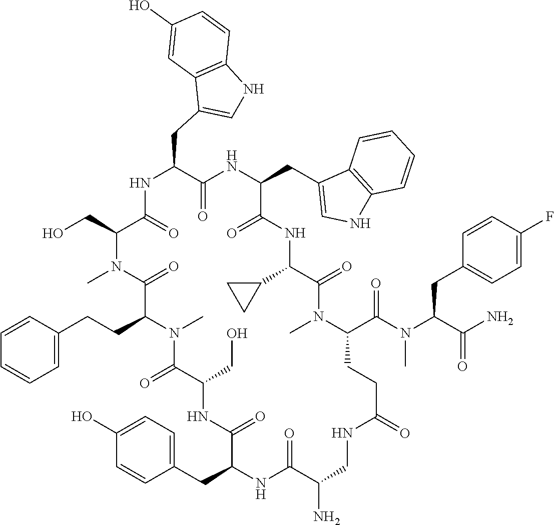 [SMILES: CN1C(=O)[C@H](CO)NC(=O)[C@H](Cc2ccc(O)cc2)NC(=O)[C@@H](N)CNC(=O)CC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@@H]1CCc1ccccc1] | Method AP-1 t_{R} = 3.88 min [M + H]⁺ = 1419.7 |

(3S,6S,9S,12S,15S,18S,21S,24S,30S)-6-((1H-indol-3-yl)methyl)-24-amino-N-
((S)-1-amino-3-(4-fluorophenyl)-1-oxopropan-2-yl)-3-cyclopropyl-9-((5-
hydroxy-1H-indol-3-yl)methyl)-21-(4-hydroxybenzyl)-12,18-
bis(hydroxymethyl)-N,1,13,16-tetramethyl-2,5,8,11,14,17,20,23,27-nonaoxo-
15-phenethyl-1,4,7,10,13,16,19,22,26-nonaazacyclotriacontane-30-
carboxamide TFA salt

### 2.1.4 Synthesis of (3S,6S,9S,12S,15S,18S,21S,24S,27S)-9-((1H-indol-3-yl)methyl)-27-amino-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-15,21-bis(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamide TFA 5 salt (Example A35 (A35-2 disclosed as SEQ ID NO: 111 and A35 disclosed as SEQ ID NO: 12))

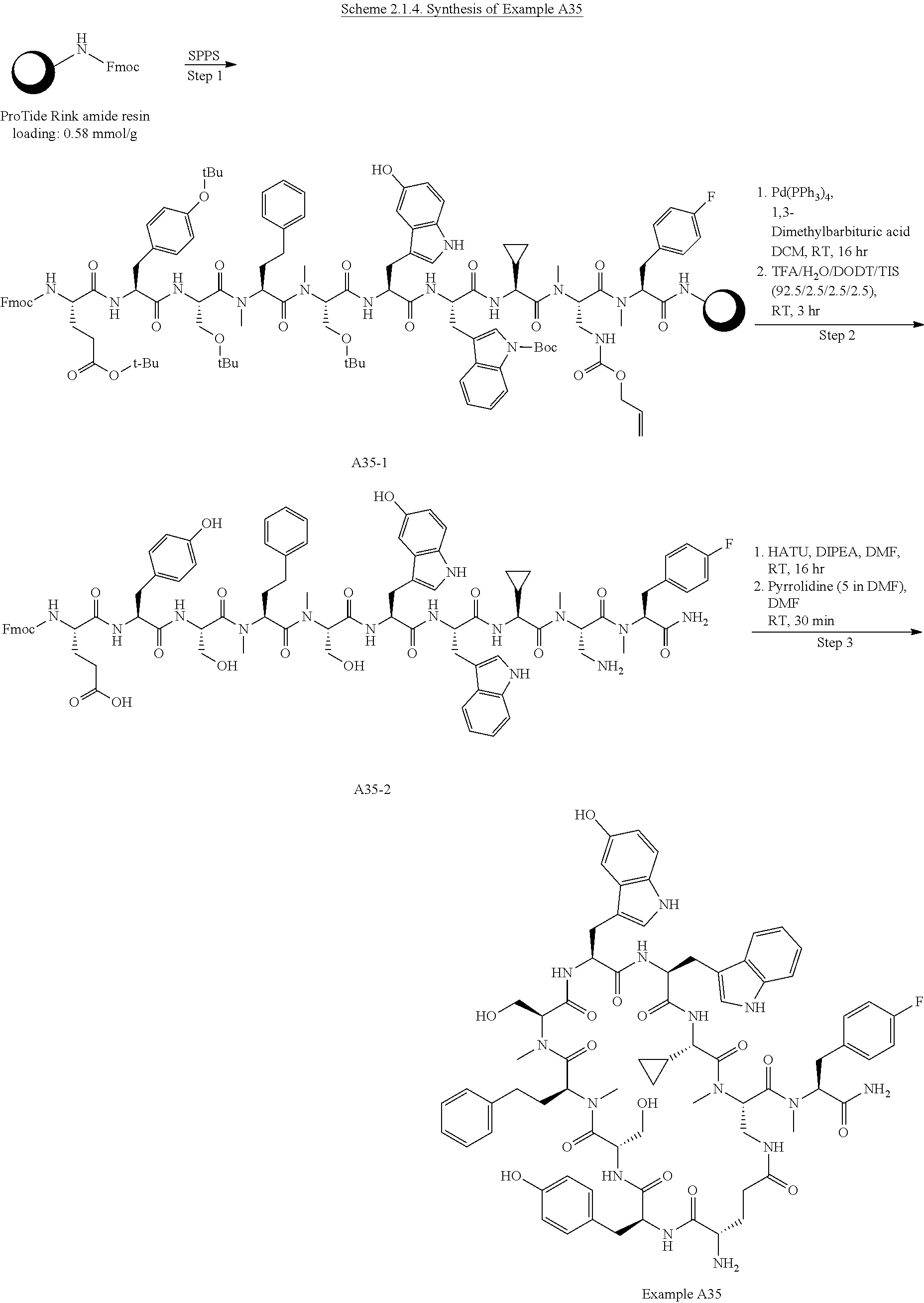

### Step 1. A35-1

The assembly of the linear peptide was done on the CEM Liberty Blue Synthesizer using commercial Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×4 min at 90° C.
Method B Fmoc removal: 2×1 min at 90° C./Coupling: 2×4 min at 90° C.

After the assembly of the linear peptide the resin was washed with DMF (5×) and DCM (5×). The amino acids and coupling methods are summarized in Table 2.7.

TABLE 2.7

| Synthesis | Amino acid | | |
|---|---|---|---|
| cycle | residue^{a} | Amino acid | Method |
| 1 | A¹⁰ | Fmoc-L-N-Me-Phe(4F)-OH | A |
| 2 | A⁹ | Fmoc-L-N-Me-Dap(Alloc)- | B |
| | | OH | |
| 3 | A⁸ | Fmoc-L-CyclopropylGly-OH | B |
| 4 | A⁷ | Fmoc-L-Trp(Boc)-OH | A |
| 5 | A⁶ | Fmoc-L-Trp(5-OH)-OH | A |
| 6 | A⁵ | Fmoc-L-N-Me-Ser(tBu)-OH | A |
| 7 | A⁴ | Fmoc-L-N-Me-Homo-Phe- | B |
| | | OH | |
| 8 | A³ | Fmoc-L-Ser(tBu)-OH | B |
| 9 | A² | Fmoc-L-Tyr(tBu)-OH | A |
| 10 | A¹ | Fmoc-L-Glu(OtBu)-OH | A |

^{a}Refers to the corresponding amino acid residue of cyclic peptide, P, as found, e.g., in Formulae (I), (Ia), (Ib), (Ic), (Id), or in the Table of Example III.

### Step 2. A35-2

Step 2-1: The resin from Step 1 (calculated with 100 μmol) was suspended with DCM (5 mL). Then Pd(PPh₃)₄ (34.7 mg, 0.3 eq., 30 μmol) followed by 1,3-Dimethylbarbituric acid (78 mg, 5 eq., 500 mmol) were added. The reaction was agitated with argon for 16 hr. The resin was drained and washed with DCM (10×), DMF (5×) and DCM (5×).

Step 2-2: To the resin from Step 2-1 (calculated with 100 μmol) was added TFA/H₂O/DODT/TIS (92.5/2.5/2.5/2.5) (4 mL) and the resin was shaken at RT for 3 hr. The crude peptide was precipitated with cold diethyl ether/heptane (1/1) (45 mL) giving a precipitate. The suspension was centrifuged, and the solvent was decanted. The product was washed with another portion of cold diethyl ether/heptane (1/1) (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A35-2 (26 mg, 12 μmol, 12% yield, TFA salt) as a white solid. LCMS Method AP-1, t_{R}=4.65 min, [M+H]⁺=1659.7.

### Step 3. Example A35

Step 3-1: A35-2 (13 mg, 1 eq., 7.33 μmol, TFA salt) was dissolved in DMF (1 mL). Then HATU (4.18 mg, 1.5 eq., 10.99 μmol), followed by DIPEA (2.84 mg, 3.8 μL, 3 eq., 22 μmol) were added. The reaction mixture was stirred at RT for 16 hr.

Step 3-2: The reaction mixture from Step 3-1 was directly treated with pyrrolidine (5% in DMF) (300 μL, 25 eq., 182.7 μmol) and shaken at RT for 30 min, then quenched with ACN/water (1/1). The product was isolated by preparative RP HPLC (Column: XSelect® Peptide CSH C18, OBD™ Prep Column, 130 Å, 5 μm, 30 mm×250 mm; Eluent A: H₂O+0.1% TFA and Eluent B: ACN). Pure fractions were combined and lyophilized to afford Example A35 (4.3 mg, 2.58 μmol, 35% yield, TFA salt) as a white solid. LCMS Method AP-1, t_{R}=3.78 min, [M+H]⁺=1419.7.

The compounds in Table 2.8 were synthesized in analogy to Example A35.

TABLE 2.8
Examples synthesized in analogy to Example A35 (Scheme 2.1.4).

| Ex. No. | Structure/Example Name | LCMS |
|---|---|---|
| A36 | 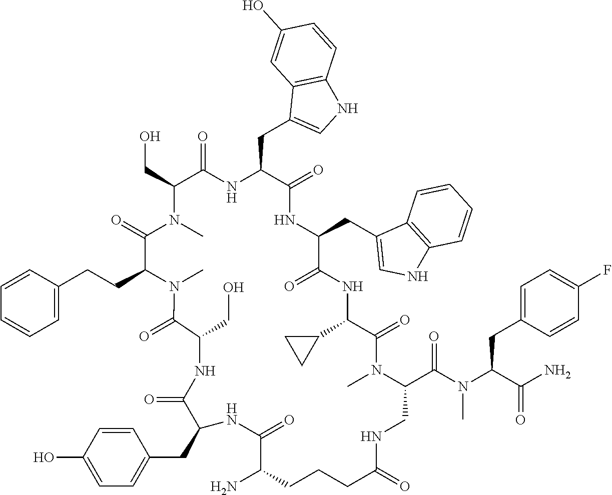 | Method AP-1 t_{R} = 3.95 min [M + H]⁺ = 1433.7 |
| | (3S,6S,9S,12S,15S,18S,21S,24S,27S)-9-((1H-indol-3-yl)methyl)-27-amino-N- | |
| | ((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-15,21- | |
| | bis(hydroxymethyl)-N,4,16,19-tetramethyl-5,8,11,14,17,20,23,26,31-nonaoxo- | |
| | 18-phenethyl-1,4,7,10,13,16,19,22,25-nonaazacyclohentriacontane-3- | |
| | carboxamide TFA salt | |
| A37 (SEQ ID NO: 13) | 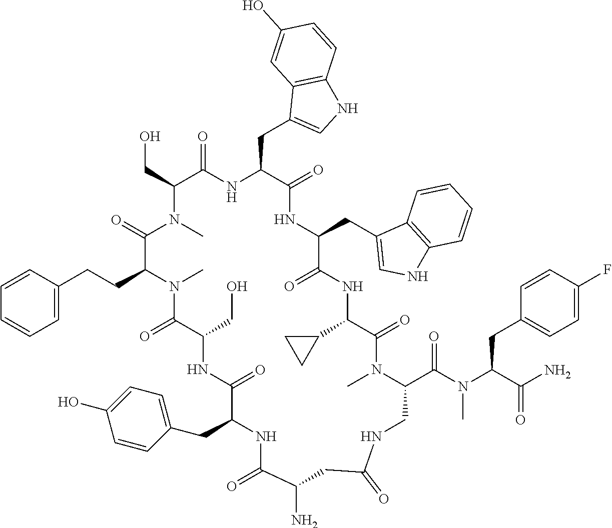 | Method AP-1 t_{R} = 3.68 min [M + H]⁺ = 1405.6 |
| | (3S,6S,9S,12S,15S,18S,21S,24S,27S)-9-((1H-indol-3-yl)methyl)-27-amino-N- | |
| | ((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-15,21- | |
| | bis(hydroxymethyl)-N,4,16,19-tetramethyl-5,8,11,14,17,20,23,26,29-nonaoxo- | |
| | 18-phenethyl-1,4,7,10,13,16,19,22,25-nonaazacyclononacosane-3- | |
| | carboxamide TFA salt | |
| A38 | 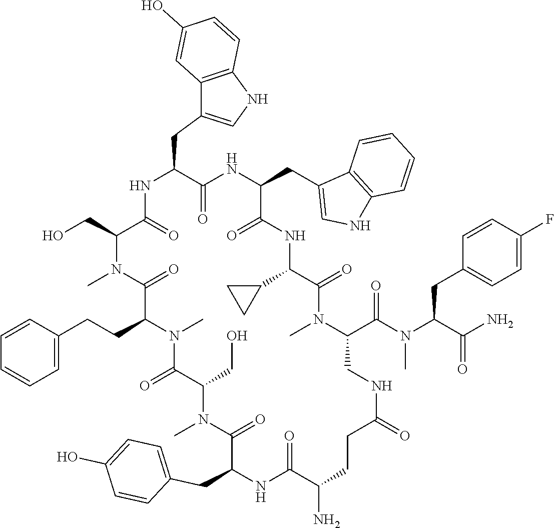 [SMILES: CN1C(=O)[C@H](Cc2ccc(O)cc2)NC(=O)[C@@H](N)CCC(=O)NC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@H](CCc2ccccc2)N(C)C(=O)[C@@H]1CO] | Method AP-1 TR = 3.92 min [M + H]⁺ = 1433.7 |
| | (3S,6S,9S,12S,15S,18S,21S,24S,27S)-9-((1H-indol-3-yl)methyl)-27-amino-N- | |
| | ((S)-1-amino-3-(4-fluorophenyl)-1-oxopropan-2-yl)-6-cyclopropyl-12-((5- | |
| | hydroxy-1H-indol-3-yl)methyl)-24-(4-hydroxybenzyl)-15,21- | |
| | bis(hydroxymethyl)-N,4,16,19,22-pentamethyl-5,8,11,14,17,20,23,26,30- | |
| | nonaoxo-18-phenethyl-1,4,7,10,13,16,19,22,25-nonaazacyclotriacontane-3- | |
| | carboxamide TFA salt | |
| A39 | 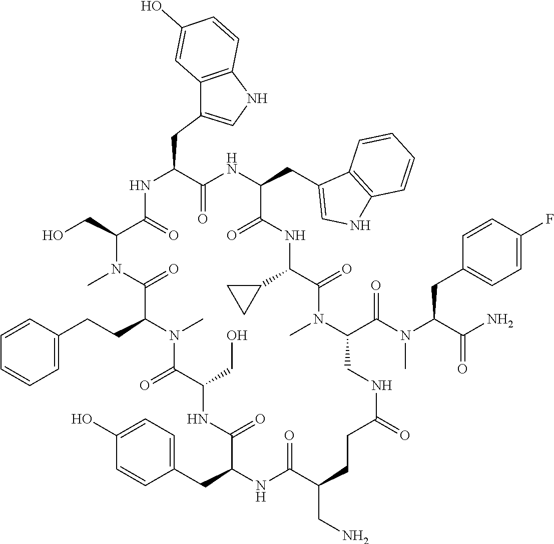 [SMILES: CN1C(=O)[C@H](CO)NC(=O)[C@H](Cc2ccc(O)cc2)NC(=O)[C@H](CN)CCC(=O)NC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@@H]1CCc1ccccc1] | Method AP-1 t_{R} = 3.90 min [M + H]⁺ = 1433.7 |

(3S,6S,9S,12S,15S,18S,21S,24S,27S)-9-((1H-indol-3-yl)methyl)-N-((S)-1-
amino-3-(4-fluorophenyl)-1-oxopropan-2-yl)-27-(aminomethyl)-6-
cyclopropyl-12-((5-hydroxy-1H-indol-3-yl)methyl)-24-(4-hydroxybenzyl)-
15,21-bis(hydroxymethyl)-N,4,16,19-tetramethyl-5,8,11,14,17,20,23,26,30-
nonaoxo-18-phenethyl-1,4,7,10,13,16,19,22,25-nonaazacyclotriacontane-3-
carboxamide TFA salt

### 2.1.5 Synthesis of (3S,6S,9S,12S,15S,18S,21S,24S,27S)-9-((1H-indol-3-yl)methyl)-27-acetamido-15-(4-acetamidobutyl)-N—((S)-1-(((S)-1-amino-6-azido-1-oxohexan-2-yl)amino)-3-(4-fluorophenyl)-1-oxopropan-2-yl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide (Example A74) and (3S,6S,9S,12S,15S,18S,21S,24S,27S)-9-((1H-indol-3-yl)methyl)-27-acetamido-15-(4-acetamidobutyl)-6-cyclopropyl-N—((S)-1-(((S)-1,6-diamino-1-oxohexan-2-yl)amino)-3-(4-fluorophenyl)-1-oxopropan-2-yl)-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide TFA salt (Example A40 (A40 disclosed as SEQ ID NO: 14))

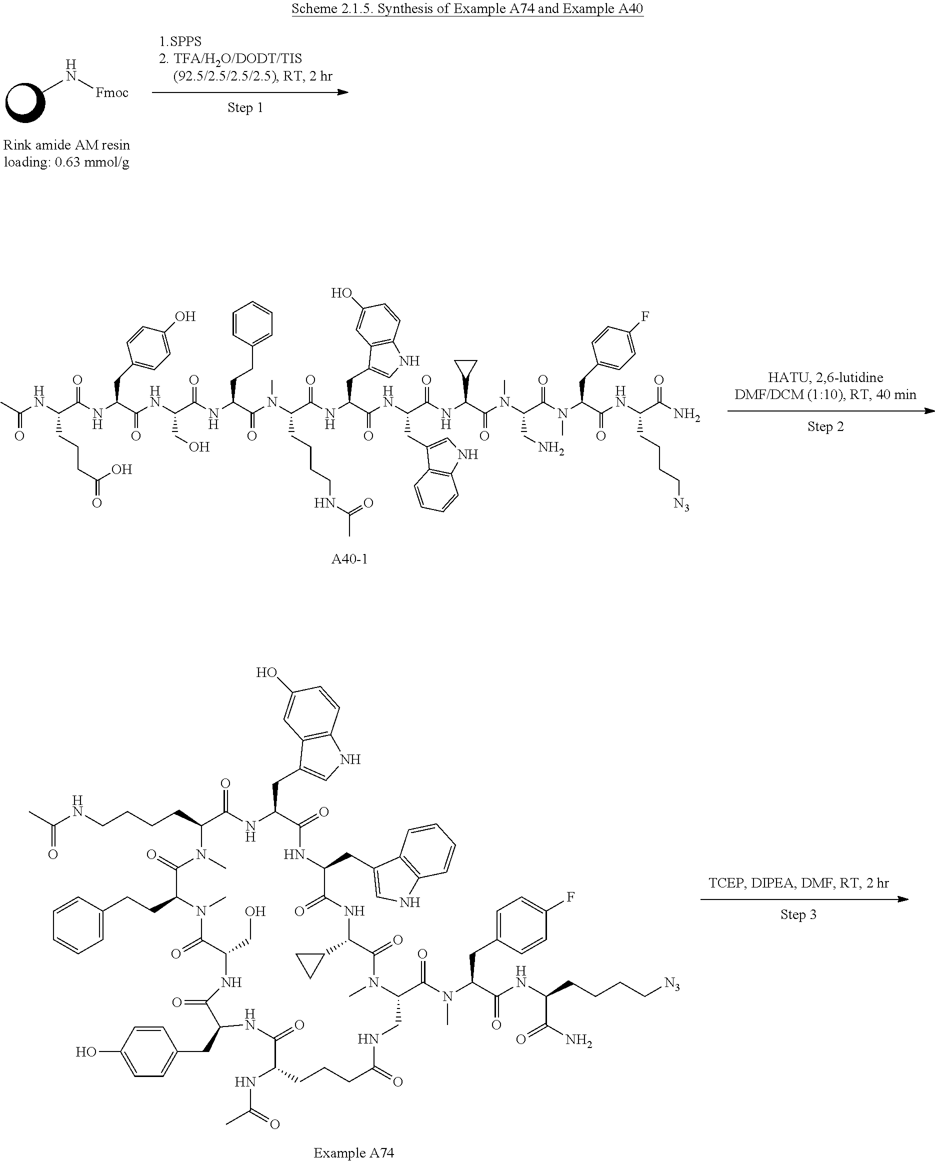

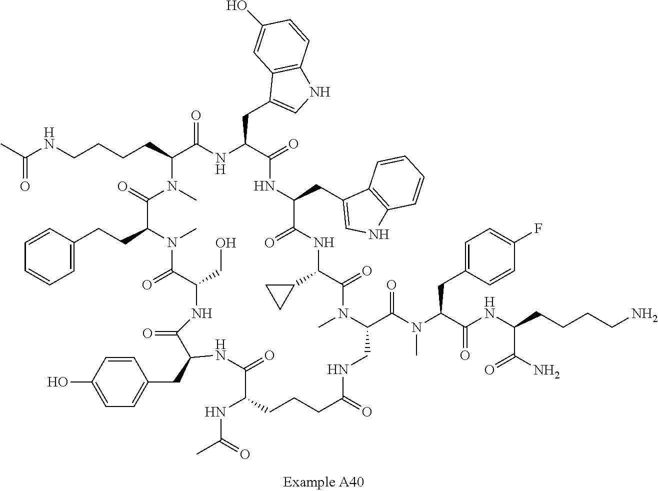 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCN)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

### Step 1. A40-1

Step 1-1: The assembly of the linear peptide was done on the CEM Liberty Prime Synthesizer using commercial Rink amide AM resin R2 (loading 0.63 mmol/g, 794 mg, 0.500 mmol). The couplings were carried out using the following solutions: Fmoc-amino acid (0.5 M solution DMF, 4 mL, 4 eq.), DIC (4 M solution in DMF, 1 mL, 8 eq.), Oxyma Pure® (0.25 M solution in DMF, 8 mL, 4 eq.), addition by synthesizer. Fmoc removal was performed using a solution of pyrrolidine (25% in DMF, 2.5 mL), addition by synthesizer.

The amino acids were coupled with a set of different methods.
Method A Fmoc removal: 1×1 min at 110° C./Coupling: 1×4 min at 105° C.
Method B Fmoc removal: 2×10 min at RT/Coupling: 2×4 min at 105° C.

After the assembly of the linear peptide the resin was washed with DMF (5×) and DCM (5×). The amino acids and coupling methods are summarized in Table 2.9.

TABLE 2.9

| Synthesis | Amino acid | | |
|---|---|---|---|
| cycle | residue^{a} | Amino acid | Method |
| 1 | A¹⁰* | Fmoc-L-Lys(N3)-OH | A |
| 2 | A¹⁰ | Fmoc-L-N-Me-Phe(4F)-OH | A |
| 3 | A⁹ | Fmoc-L-N-Me-Dap(Boc)- | B |
| | | OH | |
| 4 | A⁸ | Fmoc-L-CyclopropylGly-OH | B |
| 5 | A⁷ | Fmoc-L-Trp(Boc)-OH | B |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe- | B |
| | | OH | |
| 9 | A³ | Fmoc-L-Ser(tBu)-OH | B |
| 10 | A² | Fmoc-L-Tyr(tBu)-OH | B |
| 11 | A¹ | Fmoc-L-Homo-Glu(OtBu)- | A |
| | | OH | |
| 12 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (20 mL) and the resin was shaken at RT for 3 hr. The crude peptide was precipitated with cold heptane/MTBE (1/1) (180 mL). The suspension was centrifuged, and the solvent was decanted. The product was washed with another portion of cold heptane/MTBE (160 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A40-1 (194 mg, 93.0 μmol, 19% yield, TFA salt) as a white solid. LCMS Method AP-1, t_{R}=4.07 min, [M+H]⁺=1731.0.

### Step 2. Example A74

Step 2-1: A40-1 (194 mg, 1 eq., 0.105 mmol, TFA salt) was dissolved in DMF/DCM (1/10) (165 mL). Then 2,6-lutidine (281.7 mg, 305 μL, 25 eq., 2.629 mmol) was added. A solution of HATU (43.98 mg, 1.1 eq., 0.116 mmol) in DMF (1 mL) was added dropwise. The reaction mixture was stirred at RT for 1 hr, then concentrated at 30° C. in vacuo to remove the DCM and part of DMF. The residue was precipitated with cold MTBE (100 mL). The suspension was centrifuged, and the solvent was decanted. The product was washed with another portion of cold MTBE (100 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A74 (93.5 mg, 0.054 mmol, 51% yield) as a white solid. LCMS Method AP-1, t_{R}=4.96 min, [M+H]⁺=1713.8.

### Step 3. Example A40

Example A74 (127 mg, 1 eq., 74.14 μmol) (two batches of A⁷⁴ synthesized through different campaigns were combined) was dissolved in DMF (2.00 mL), then TCEP (85.0 mg, 4 eq., 296.6 μmol, HCl salt) and DIPEA (57.5 mg, 77.5 μL, 6 eq., 444.8 μmol) were added. The reaction mixture was shaken at RT for 1.5 hr. The reaction mixture was precipitated with cold MTBE (40 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A40 (121.5 mg, 67 μmol, 90% yield, TFA salt) as a white solid. LCMS Method AP-1, t_{R}=3.98 min, [M+H]⁺=1687.9.

The compounds in Table 2.10 were synthesized in analogy to Example A40.

TABLE 2.10
Examples synthesized in analogy to Example A40 (Scheme 2.1.5).

| Ex. No. | Structure/Example Name | LCMS |
|---|---|---|
| A41 (SEQ ID NO: 15) | 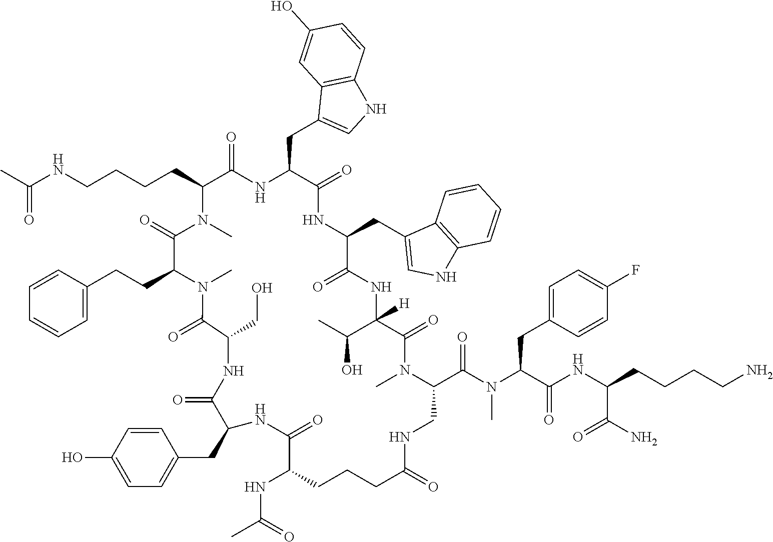 | Method AP-1 t_{R} = 3.74 min [M + H]⁺ = 1690.8 |
| | (3S,6S,9S,12S,15S,18S,21S,24S,27S)-9-((1H-indol-3-yl)methyl)-27-acetamido- | |
| | 15-(4-acetamidobutyl)-N-((S)-1-(((S)-1,6-diamino-1-oxohexan-2-y1)amino)-3- | |
| | (4-fluorophenyl)-1-oxopropan-2-yl)-12-((5-hydroxy-1H-indol-3-y1)methyl)-24- | |
| | (4-hydroxybenzyl)-6-((S)-1-hydroxyethyl)-21-(hydroxymethyl)-N,4,16,19- | |
| | tetramethyl-5,8,11,14,17,20,23,26,31-nonaoxo-18-phenethyl- | |
| | 1,4,7,10,13,16,19,22,25-nonaazacyclohentriacontane-3-carboxamide TFA salt | |
| A42 | 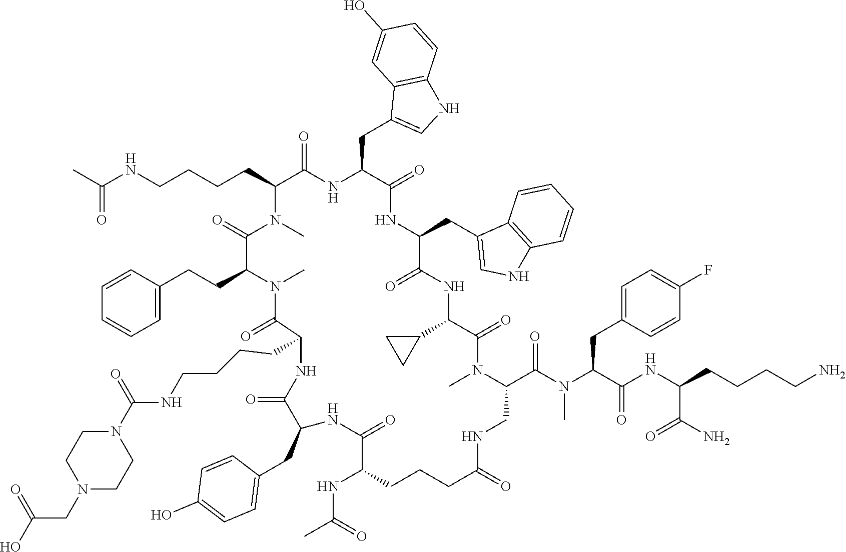 | Method AP-2 t_{R} = 7.73 min [M + H]⁺ = 1899.0 |
| | 2-(4-((4-((2S,5S,8S,11S,14S,17S,20S,23S,30S)-17-((1H-indol-3-yl)methyl)-30- | |
| | acetamido-11-(4-acetamidobutyl)-20-cyclopropyl-23-(((S)-1-(((S)-1,6-diamino- | |
| | 1-oxohexan-2-y1)amino)-3-(4-fluorophenyl)-1-oxopropan-2- | |
| | yl)(methyl)carbamoyl)-14-((5-hydroxy-1H-indol-3-yl)methyl)-2-(4- | |
| | hydroxybenzyl)-7,10,22-trimethyl-3,6,9,12,15,18,21,26,31-nonaoxo-8- | |
| | phenethyl-1,4,7,10,13,16,19,22,25-nonaazacyclohentriacontan-5- | |
| | yl)butyl)carbamoyl)piperazin-1-yl)acetic acid TFA salt | |
| A43 (SEQ ID NO: 16) | 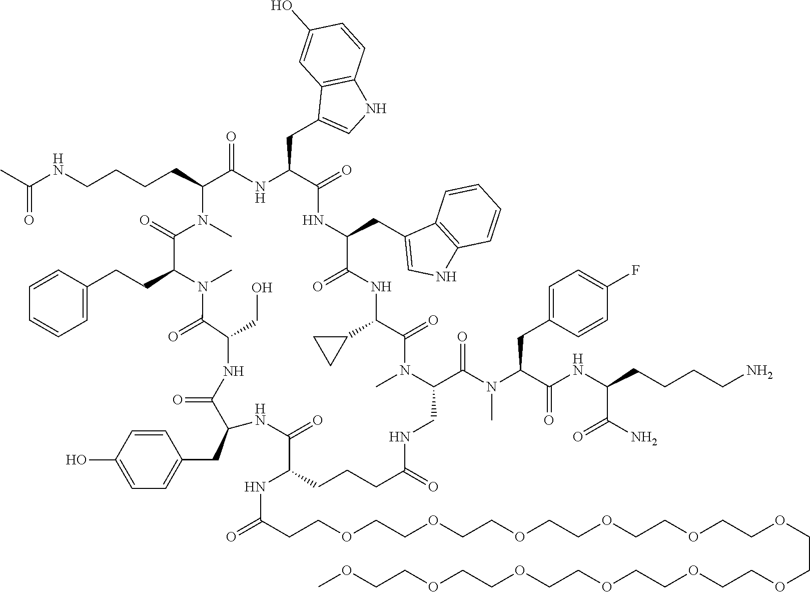 | Method AP-1 t_{R} = 4.36 min [M + H]⁺ = 2216.2 |
| | (3S,6S,9S,12S,15S,18S,21S,24S,27S)-9-((1H-indol-3-y1)methyl)-15-(4- | |
| | acetamidobutyl)-6-cyclopropyl-N-((S)-1-(((S)-1,6-diamino-1-oxohexan-2- | |
| | yl)amino)-3-(4-fluorophenyl)-1-oxopropan-2-yl)-12-((5-hydroxy-1H-indol-3- | |
| | yl)methyl)-24-(4-hydroxybenzyl)-21-(hydroxymethyl)-N,4,16,19-tetramethyl- | |
| | 27-(2,5,8,11,14,17,20,23,26,29,32,35-dodecaoxaoctatriacontan-38-amido)- | |
| | 5,8,11,14,17,20,23,26,31-nonaoxo-18-phenethyl-1,4,7,10,13,16,19,22,25- | |
| | nonaazacyclohentriacontane-3-carboxamide TFA salt | |
| A44 | 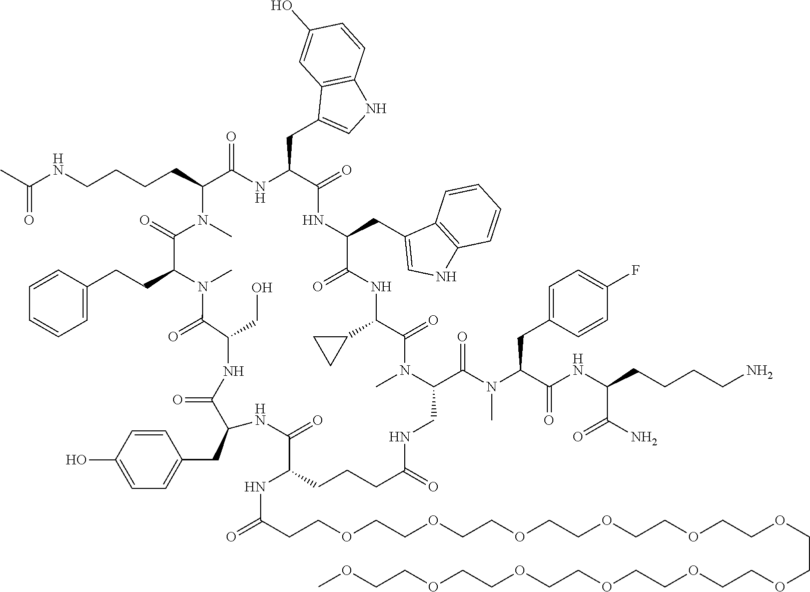 | Method AP-1 t_{R} = 5.29 min [M + H]⁺ = 2242.2 |
| | (3S,6S,9S,12S,15S,18S,21S,24S,27S)-9-((1H-indol-3-yl)methyl)-15-(4- | |
| | acetamidobutyl)-N-((S)-1-(((S)-1-amino-6-azido-1-oxohexan-2-yl)amino)-3-(4- | |
| | fluorophenyl)-1-oxopropan-2-y1)-6-cyclopropyl-12-((5-hydroxy-1H-indol-3- | |
| | yl)methyl)-24-(4-hydroxybenzyl)-21-(hydroxymethyl)-N,4,16,19-tetramethyl- | |
| | 27-(2,5,8,11,14,17,20,23,26,29,32,35-dodecaoxaoctatriacontan-38-amido)- | |
| | 5,8,11,14,17,20,23,26,31-nonaoxo-18-phenethyl-1,4,7,10,13,16,19,22,25- | |
| | nonaazacyclohentriacontane-3-carboxamide | |
| A45 (SEQ ID NO: 17) | 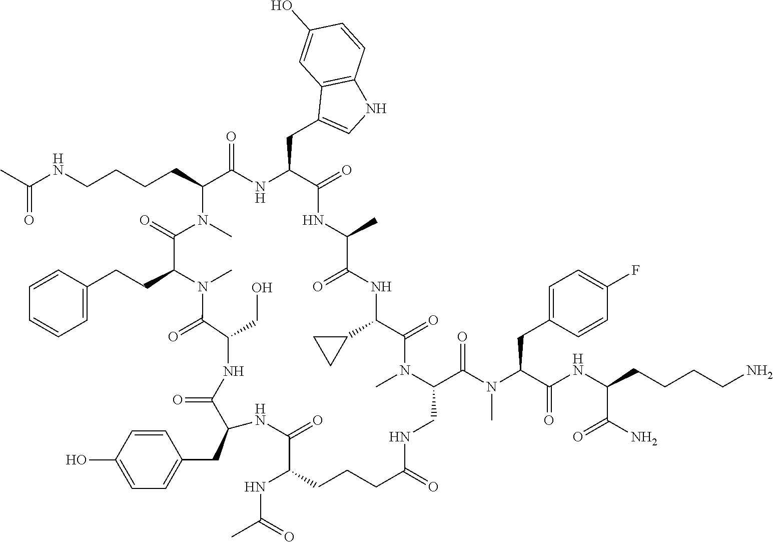 | Method AP-1 t_{R} = 3.47 min [M + H]⁺ = 1571.8 |
| | (3S,6S,9S,12S,15S,18S,21S,24S,27S)-27-acetamido-15-(4-acetamidobutyl)-6- | |
| | cyclopropyl-N-((S)-1-(((S)-1,6-diamino-1-oxohexan-2-y1)amino)-3-(4- | |
| | fluorophenyl)-1-oxopropan-2-y1)-12-((5-hydroxy-1H-indol-3-yl)methyl)-24-(4- | |
| | hydroxybenzyl)-21-(hydroxymethyl)-N,4,9,16,19-pentamethyl- | |
| | 5,8,11,14,17,20,23,26,31-nonaoxo-18-phenethyl-1,4,7,10,13,16,19,22,25- | |
| | nonaazacyclohentriacontane-3-carboxamide TFA salt | |
| A46 | 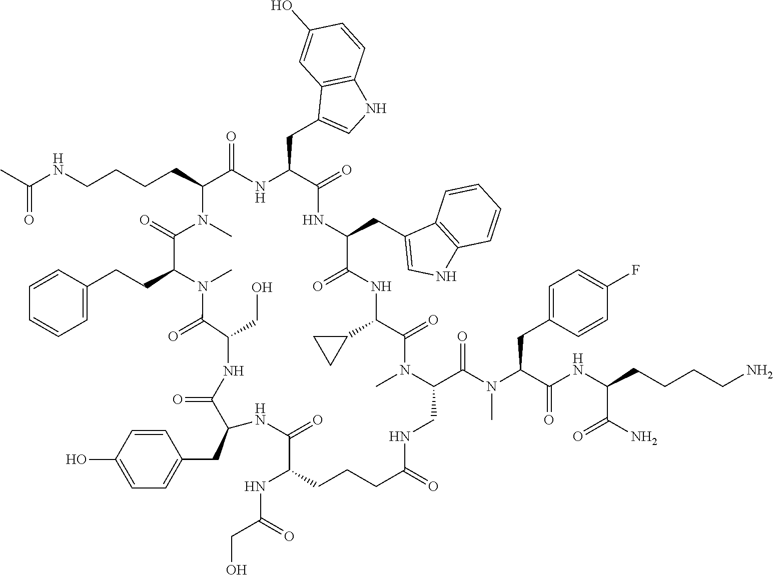 | Method AP-1 t_{R} = 4.82 min [M + H]⁺ = 1728.8 |
| | (3S,6S,9S,12S,15S,18S,21S,24S,27S)-9-((1H-indol-3-yl)methyl)-15-(4- | |
| | acetamidobutyl)-N-((S)-1-(((S)-1-amino-6-azido-1-oxohexan-2-yl)amino)-3-(4- | |
| | fluorophenyl)-1-oxopropan-2-y1)-6-cyclopropyl-12-((5-hydroxy-1H-indol-3- | |
| | yl)methyl)-27-(2-hydroxyacetamido)-24-(4-hydroxybenzyl)-21- | |
| | (hydroxymethyl)-N,4,16,19-tetramethyl-5,8,11,14,17,20,23,26,31-nonaoxo-18- | |
| | phenethyl-1,4,7,10,13,16,19,22,25-nonaazacyclohentriacontane-3-carboxamide | |
| A47 | 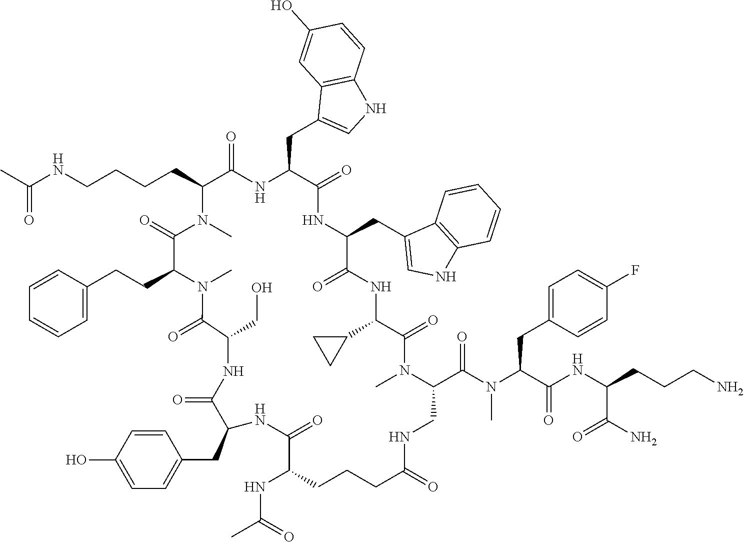 | Method AP-1 t_{R} = 3.77 min [M + H]⁺ = 1672.8 |
| | (3S,6S,9S,12S,15S,18S,21S,24S,27S)-9-((1H-indol-3-y1)methyl)-27-acetamido- | |
| | 15-(4-acetamidobutyl)-6-cyclopropyl-N-((S)-1-(((S)-1,5-diamino-1-oxopentan-2- | |
| | yl)amino)-3-(4-fluorophenyl)-1-oxopropan-2-y1)-12-((5-hydroxy-1H-indol-3- | |
| | yl)methyl)-24-(4-hydroxybenzyl)-21-(hydroxymethyl)-N,4,16,19-tetramethyl- | |
| | 5,8,11,14,17,20,23,26,31-nonaoxo-18-phenethyl-1,4,7,10,13,16,19,22,25- | |
| | nonaazacyclohentriacontane-3-carboxamide TFA salt | |
| A48 (SEQ ID NO: 18) | 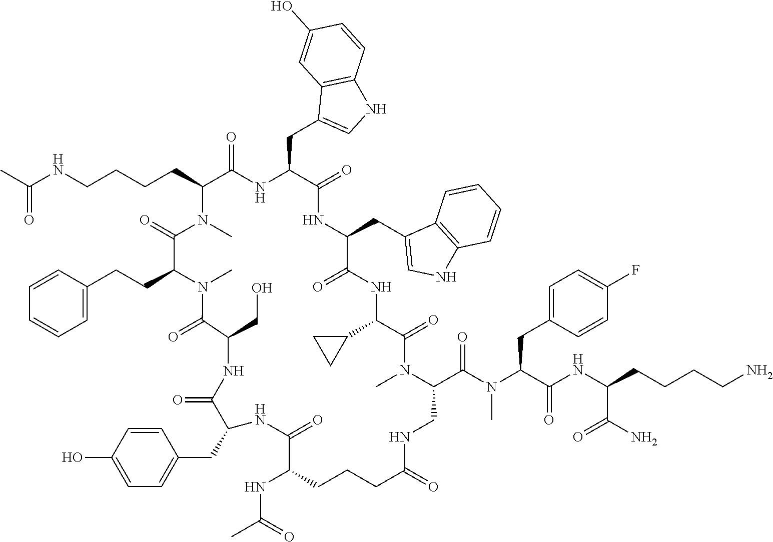 | Method AP-1 t_{R} = 4.03 min [M + H]⁺ = 1687.9 |
| | (3S,6S,9S,12S,15S,18S,21R,24R,27S)-9-((1H-indol-3-yl)methyl)-27-acetamido- | |
| | 15-(4-acetamidobutyl)-6-cyclopropyl-N-((S)-1-(((S)-1,6-diamino-1-oxohexan-2- | |
| | yl)amino)-3-(4-fluorophenyl)-1-oxopropan-2-y1)-12-((5-hydroxy-1H-indol-3- | |
| | yl)methyl)-24-(4-hydroxybenzyl)-21-(hydroxymethyl)-N,4,16,19-tetramethyl- | |
| | 5,8,11,14,17,20,23,26,31-nonaoxo-18-phenethyl-1,4,7,10,13,16, 19,22,25- | |
| | nonaazacyclohentriacontane-3-carboxamide TFA salt | |
| A75 (SEQ ID NO: 33) | 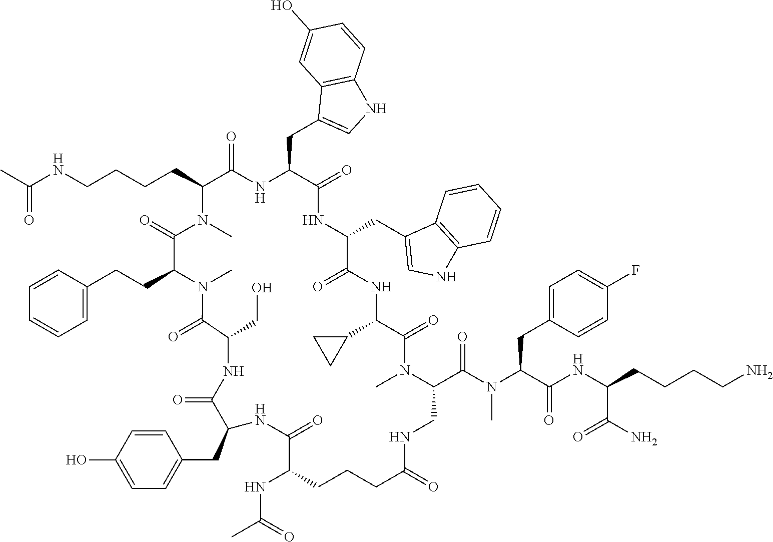 | Method AP-1 t_{R} = 3.83 min [M + H]⁺ = 1687.8 |
| | (3S,6S,9R,12S,15S,18S,21S,24S,27S)-9-((1H-indol-3-yl)methyl)-27-acetamido- | |
| | 15-(4-acetamidobutyl)-6-cyclopropyl-N-((S)-1-(((S)-1,6-diamino-1-oxohexan-2- | |
| | yl)amino)-3-(4-fluorophenyl)-1-oxopropan-2-y1)-12-((5-hydroxy-1H-indol-3- | |
| | yl)methyl)-24-(4-hydroxybenzyl)-21-(hydroxymethyl)-N,4,16,19-tetramethyl- | |
| | 5,8,11,14,17,20,23,26,31-nonaoxo-18-phenethyl-1,4,7,10,13,16,19,22,25- | |
| | nonaazacyclohentriacontane-3-carboxamide TFA salt | |

### 2.1.6 Synthesis of (6S,9S,12S,15S,18S,21S,24S,27S,30S)-12-((1H-indol-3-yl)methyl)-30-acetamido-18-(4-acetamidobutyl)-N—((S)-1-(((S)-1-amino-6-azido-1-oxohexan-2-yl)amino)-3-(4-fluorophenyl)-1-oxopropan-2-yl)-9-cyclopropyl-15-((5-hydroxy-1H-indol-3-yl)methyl)-27-(4-hydroxybenzyl)-24-(hydroxymethyl)-N,7,19,22-tetramethyl-3,8,11,14,17,20,23,26,29-nonaoxo-21-phenethyl-1-oxa-4,7,10,13,16,19,22,25,28-nonaazacyclohentriacontane-6-carboxamide (Example A49 (A49-2 disclosed as SEQ ID NO: 112))

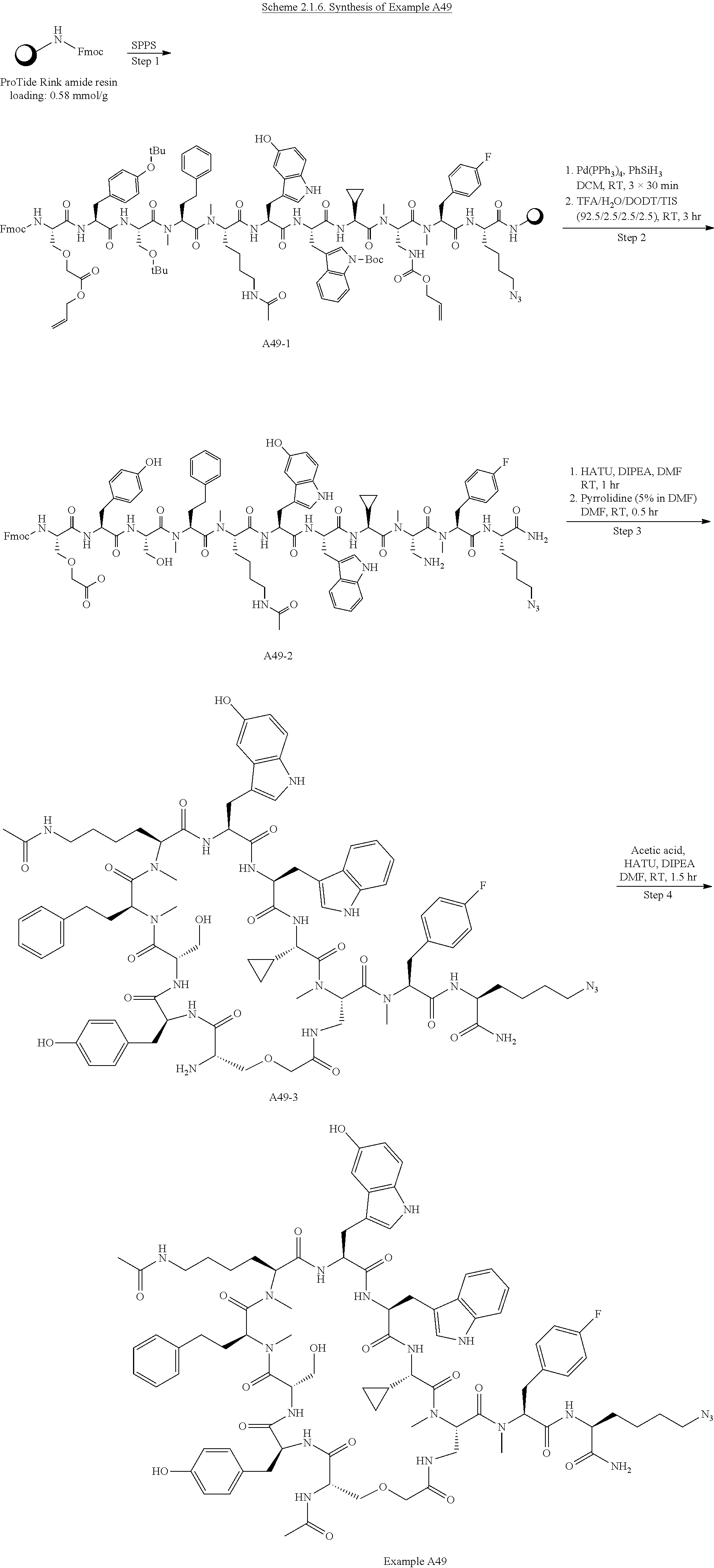

### Step 1. A49-1

The assembly of the linear peptide was done on the CEM Liberty Blue Synthesizer and by manual coupling using commercial Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×2 min at 90° C.
Method B Fmoc removal: 2×1 min at 90° C./Coupling: 2×2 min at 90° C.

Fmoc deprotection (after Synthesis cycle 10) was performed using 5% pyrrolidine in DMF (4 mL), addition by synthesizer, 1×1 min at 90° C.

### Method C (Manual Coupling)

The resin was treated with a pre-activated solution of BB2 (63.8 mg, 1.5 eq., 150 μmol), HATU (68.4 mg, 1.8 eq., 180 μmol) and DIPEA (64.6 mg, 87.1 μL, 5 eq., 500 μmol) in DMF (4 mL) and mixed at RT for 16 hr.

After the assembly of the linear peptide the resin was washed with DMF (5×) and DCM (5×). The amino acids and coupling methods are summarized in Table 2.11.

TABLE 2.11

| | Amino | | |
|---|---|---|---|
| Synthesis | acid | | |
| cycle | residue^{a} | Amino acid | Method |
| 1 | A¹⁰* | Fmoc-L-Lys(N3)-OH | A |
| 2 | A¹⁰ | Fmoc-L-N-Me-Phe(4F)-OH | A |
| 3 | A⁹ | Fmoc-L-N-Me-Dap(Alloc)-OH | B |
| 4 | A⁸ | Fmoc-L-CyclopropylGly-OH | B |
| 5 | A⁷ | Fmoc-L-Trp(Boc)-OH | A |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | A + Fmoc |
| | | | deprotection |
| 11 | A¹ | BB2 | C (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

### Step 2. A49-2

Step 2-1: The resin from Step 1 (calculated with 100 μmol) was suspended with DCM (3 mL) and treated with phenylsilane (216 mg, 248 μL, 20 eq., 2 mmol). The suspension was shaken at RT for 10 min while purged with argon. A clear solution of Pd(PPh₃)₄ (11.6 mg, 0.1 eq., 10.0 μmol) in DCM (1 mL) was added at RT. The reaction was agitated with argon for 30 min. The resin was drained, washed with DMA (3×) and DCM (5×), and the procedure was repeated twice. The resin was drained and washed with DMA (3×) and DCM (5×).

Step 2-2: To the resin from Step 2-1 was added TFA/H₂O/DODT/TIS (92.5/2.5/2.5/2.5) (4 mL) and the resin was shaken at RT for 3 hr. The crude peptide was precipitated with cold diethyl ether/heptane (1/1) (45 mL) giving a precipitate. The suspension was centrifuged, and the solvent was decanted. The product was washed with another portion of cold diethyl ether/heptane (1/1) (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A49-2 (33 mg, 15 μmol, 15% yield, TFA salt) as a white solid. LCMS Method AP-1, t_{R}=4.98 min, [M+H]=1913.9.

### Step 3. A49-3

Step 3-1: A49-2 (33 mg, 1 eq., 16.28 μmol, TFA salt) was dissolved in DMF (8 mL). Then HATU (6.8 mg, 1.1 eq., 17.91 μmol), followed by DIPEA (16.83 mg, 22.7 μL, 8 eq., 130.2 μmol) were added. The reaction mixture was stirred at RT for 1 hr.

Step 3-2: The reaction mixture from Step 3-1 was directly treated with pyrrolidine (5% in DMF) (300 μL, 11 eq., 183 μmol) and shaken at RT for 30 min. The reaction mixture was directly purified and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A49-3 (13 mg, 5.6 μmol, 34% yield, TFA salt) as a white solid. LCMS Method AP-1, t_{R}=4.32 min, [M+H]=1672.8.

### Step 4. Example A49

A49-3 (13 mg, 1 eq., 7.771 μmol, TFA salt) was dissolved in DMF (4.5 mL). Then HATU (4.4 mg, 1.5 eq., 11.66 μmol) and acetic acid (0.2 M in DMF) (50.51 μL, 1.3 eq., 10.1 μmol), followed by DIPEA (10.04 mg, 13.5 μL, 10 eq., 77.71 μmol) were added. The reaction mixture was stirred at RT for 1.5 hr. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A49 (4.5 mg, 2.2 μmol, 28% yield) as a white solid. LCMS Method AP-1, t_{R}=4.93 min, [M+H]⁺=1715.8.

### 2.1.7 Synthesis of (3R,6S,9S,12S,15S,18S,21S,24S,27S)-9-((1H-indol-3-yl)methyl)-27-acetamido-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-15,21-bis(hydroxymethyl)-N,4,16,19-tetramethyl-5,8,11,14,17,20,23,26,30-nonaoxo-18-phenethyl-1-thia-4,7,10,13,16,19,22,25,29-nonaazacyclohentriacontane-3-carboxamide (Example A50)

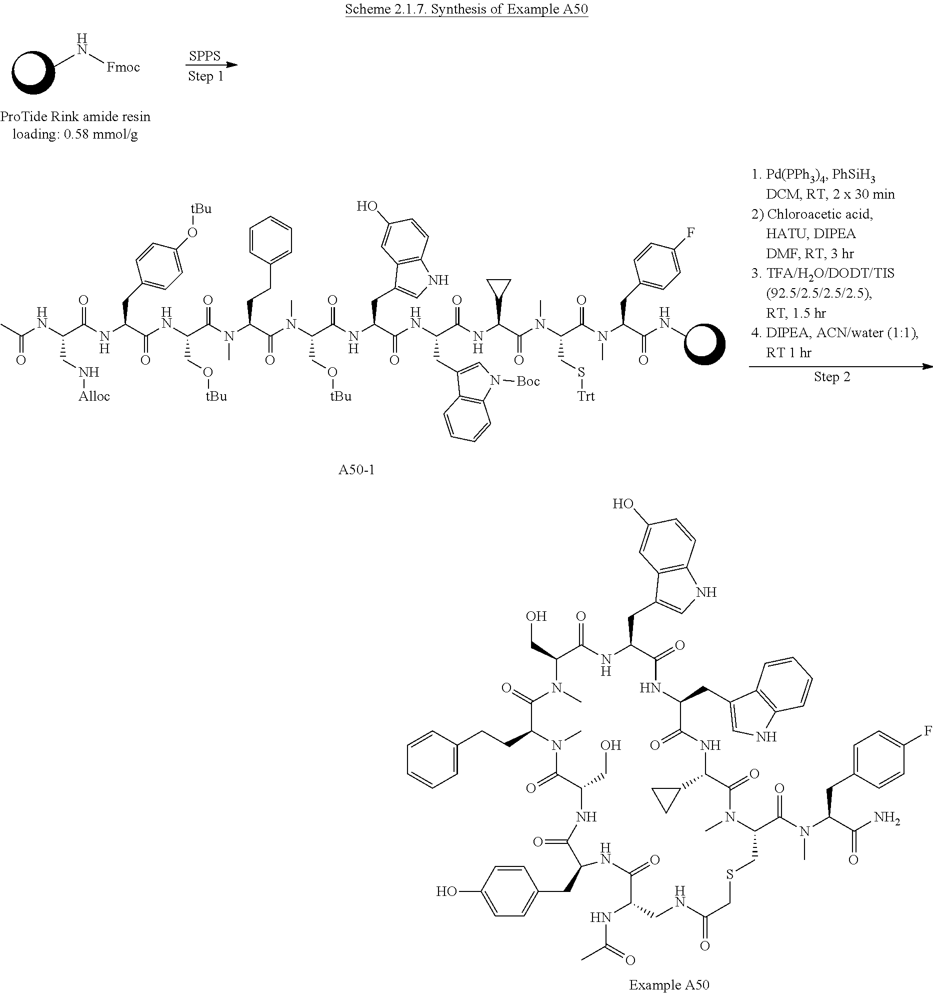

### Step 1. A50-1

The assembly of the linear peptide was done on the CEM Liberty Blue Synthesizer using Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×4 min at 90° C.
Method B Fmoc removal: 2×10 min at RT/Coupling: 2×4 min at 90° C.

After the assembly of the linear peptide the resin was washed with DMF (5×) and DCM (5×). The amino acids and coupling methods are summarized in Table 2.12.

TABLE 2.12

| Synthesis | Amino acid | | |
|---|---|---|---|
| cycle | residue^{a} | Amino acid | Method |
| 1 | A¹⁰ | Fmoc-L-N-Me-Phe(4F)-OH | A |
| 2 | A⁹ | Fmoc-L-N-Me-Cys(Trt)-OH | B |
| 3 | A⁸ | Fmoc-L-CyclopropylGly- | B |
| | | OH | |
| 4 | A⁷ | Fmoc-L-Trp(Boc)-OH | A |
| 5 | A⁶ | Fmoc-L-Trp(5-OH)-OH | A |
| 6 | A⁵ | Fmoc-L-N-Me-Ser(tBu)-OH | A |
| 7 | A⁴ | Fmoc-L-N-Me-Homo-Phe- | B |
| | | OH | |
| 8 | A³ | Fmoc-L-Ser(tBu)-OH | B |
| 9 | A² | Fmoc-L-Tyr(tBu)-OH | A |
| 10 | A¹ | Fmoc-L-Dap(Alloc)-OH | A |
| 11 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

### Step 2. Example A50

Step 2-1: The resin from Step 1 (calculated with 100 μmol) was suspended with DCM (4 mL) and treated with phenylsilane (216 mg, 248 μL, 20 eq., 2 mmol). The suspension was shaken at RT for 5 min while purged with argon. Then Pd(PPh₃)₄ (11.6 mg, 0.1 eq., 10.0 μmol) was added at RT. The reaction was agitated with argon at RT for 30 min. The reaction was agitated with argon for 30 min. The resin was drained, washed with DMA (3×) and DCM (5×), and the procedure was repeated once. The resin was drained and washed with DMF (5×) and DCM (5×).

Step 2-2: The resin from Step 2-1 was suspended with DMF (5 mL). Then chloroacetic acid (14.2 mg, 9 μL, 1.5 eq., 150 μmol), HATU (60.8 mg, 1.6 eq., 160 μmol) and DIPEA (51.7 mg, 69.7 μL, 4 eq., 400 μmol) were added. The reaction mixture was stirred at RT for 3 hr. The resin was drained and washed with DMF (5×) and DCM (5×).

Step 2-3: To the resin from Step 2-2 was added TFA/H₂O/DODT/TIS (92.5/2.5/2.5/2.5) (5 mL) and the resin was shaken at RT for 1.5 hr. The crude peptide was precipitated with cold diethyl ether (40 mL) giving a precipitate. The suspension was centrifuged, and the solvent was decanted. The product was washed with another portion of cold diethyl ether (30 mL). The suspension was centrifuged, and the solvent was decanted. The crude peptide was dried at RT under vacuo and purified by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). The pure fraction was directly used for the next Step.

Step 2-4: The product fraction from Step 2-3 was diluted with ACN/water (1/1) (20 mL) and DIPEA (38.8 mg, 52.3 μL, 3 eq., 300 μmol) was added. The reaction mixture was stirred at RT for 1 hr, then directly lyophilized to afford the crude peptide. The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A50 (10 mg, 5.8 μmol, 6% yield) as a white solid. LCMS Method AP-1, t_{R}=4.53 min, [M+H]⁺=1493.5.

The compounds in Table 2.13 were synthesized in analogy to Example A50.

TABLE 2.13
Examples synthesized in analogy to Example A50 (Scheme 2.1.7).

| Ex. No. | Structure/Example Name | LCMS |
|---|---|---|
| A51 | 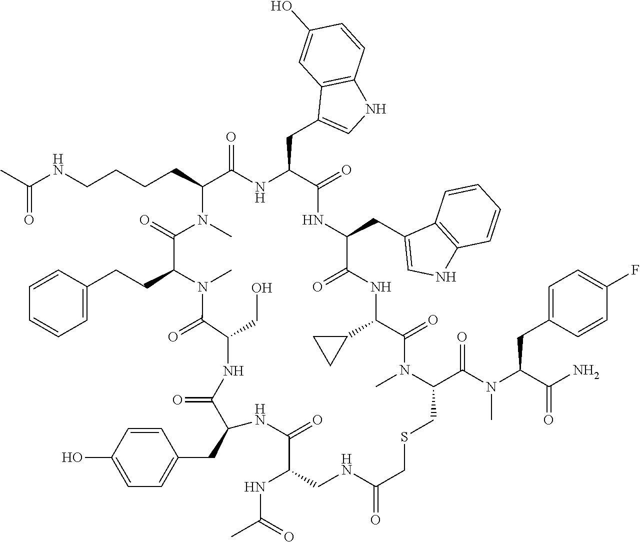 | Method AP-1 t_{R} = 4.65 min [M + H]⁺ = 1576.7 |
| | (3R,6S,9S,12S,15S,18S,21S,24S,27S)-9-((1H-indol-3-yl)methyl)-27-acetamido- | |
| | 15-(4-acetamidobutyl)-N-((S)-1-amino-3-(4-fluorophenyl)-1-oxopropan-2-y1)- | |
| | 6-cyclopropyl-12-((5-hydroxy-1H-indol-3-yl)methyl)-24-(4-hydroxybenzyl)- | |
| | 21-(hydroxymethyl)-N,4,16,19-tetramethyl-5,8,11,14,17,20,23,26,30-nonaoxo- | |
| | 18-phenethyl-1-thia-4,7,10,13,16,19,22,25,29-nonaazacyclohentriacontane-3- | |
| | carboxamide | |

### 2.1.8 Synthesis of (3R,6S,9S,12S,15S,18S,21S,24S,27S)-9-((1H-indol-3-yl)methyl)-27-amino-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-15,21-bis(hydroxymethyl)-N,4,16,19-tetramethyl-5,8,11,14,17,20,23,26-octaoxo-18-phenethyl-1-thia-4,7,10,13,16,19,22,25-octaazacyclohentriacont-29-yne-3-carboxamide TFA salt (Example A52)

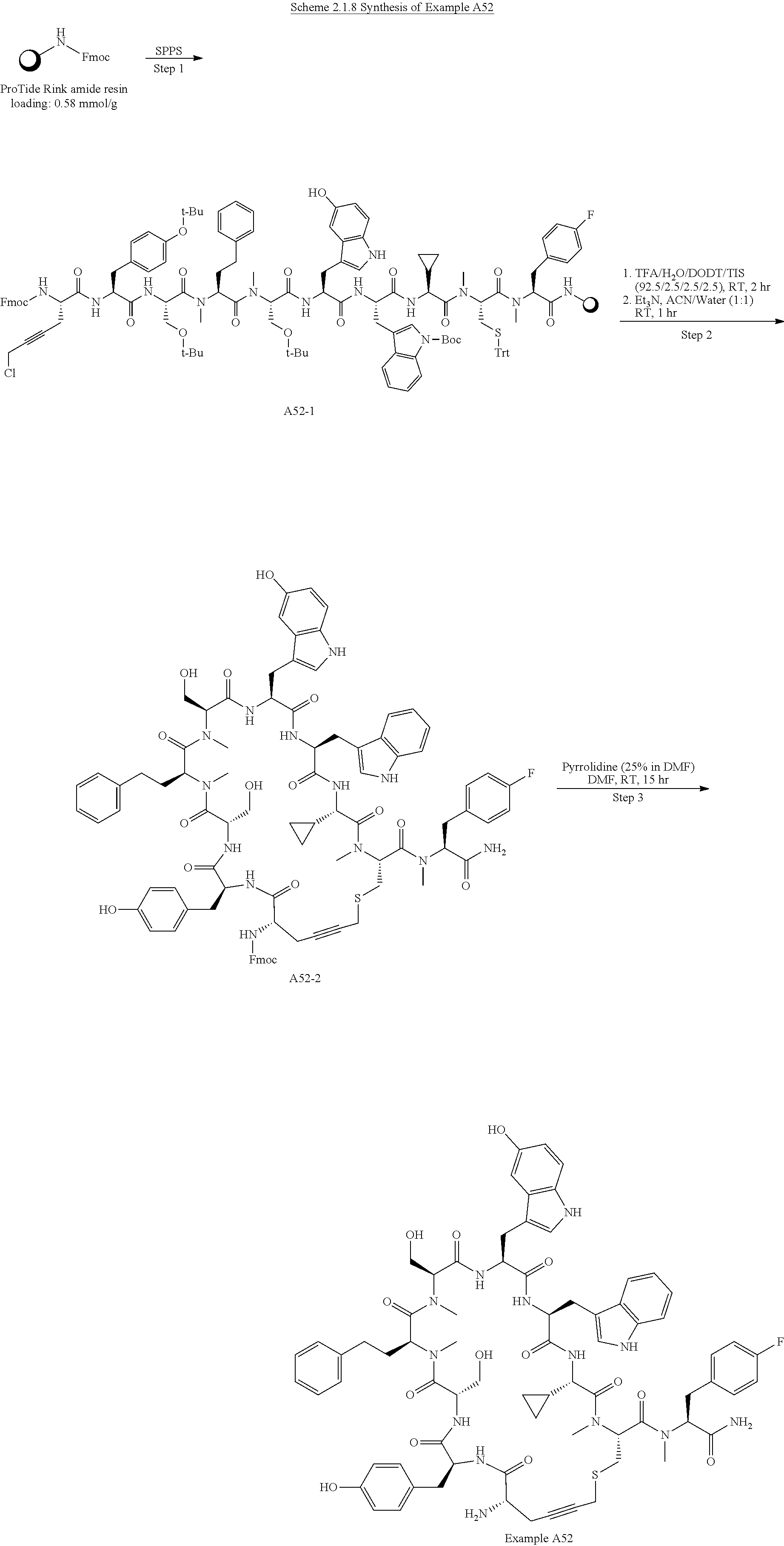

### Step 1. A52-1

The assembly of the linear peptide was done on the CEM Liberty Blue Synthesizer using Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×4 min at 90° C.
Method B Fmoc removal: 2×10 min at RT/Coupling: 2×4 min at 90° C.

After the assembly of the linear peptide the resin was washed with DMF (5×) and DCM (5×). The amino acids and coupling methods are summarized in Table 2.14.

TABLE 2.14

| Synthesis | Amino acid | | |
|---|---|---|---|
| cycle | residue^{a} | Amino acid | Method |
| 1 | A¹⁰ | Fmoc-L-N—Me-Phe(4F)—OH | A |
| 2 | A⁹ | Fmoc-L-N—Me-Cys(Trt)-OH | B |
| 3 | A⁸ | Fmoc-L-CyclopropylGly- | B |
| | | OH | |
| 4 | A⁷ | Fmoc-L-Trp(Boc)—OH | A |
| 5 | A⁶ | Fmoc-L-Trp(5-OH)—OH | A |
| 6 | A⁵ | Fmoc-L-N—Me-Ser(tBu)—OH | A |
| 7 | A⁴ | Fmoc-L-N—Me-Homo-Phe- | B |
| | | OH | |
| 8 | A³ | Fmoc-L-Ser(tBu)—OH | B |
| 9 | A² | Fmoc-L-Tyr(tBu)—OH | A |
| 10 | A¹ | Fmoc-L-Alk1-OH | A |

^{a}Refers to the corresponding amino acid residue of cyclic peptide, P, as found, e.g., in Formulae (I), (Ia), (Ib), (Ic), (Id), or in the Table of Example III.

### Step 2. A52-2

Step 2-1: To the resin from Step 1 (calculated with 100 μmol) was added TFA/H₂O/DODT/TIS (92.5/2.5/2.5/2.5) (5 mL) and the resin was shaken at RT for 2 hr. The crude peptide was precipitated with cold diethyl ether (40 mL) giving a precipitate. The suspension was centrifuged, and the solvent was decanted. The product was washed with another portion of cold diethyl ether (30 mL). The suspension was centrifuged, and the solvent was decanted. The crude peptide was dried at RT under vacuo and purified by preparative RP HPLC (Column: SunFire C18 OBD™ Prep Column, 100 Å, 5 μm, 30 mm×100 mm; Eluent A: H₂O+0.1% TFA and eluent B: ACN). Pure fractions were directly used for the next Step.

Step 2-2: The combined product fractions from Step 2-1 were diluted with ACN/water (1/1) (10 mL) and Et₃N (30.4 mg, 41.8 μL, 3 eq., 300 μmol) was added. The reaction mixture was stirred at RT for 1 hr, then directly lyophilized to afford the crude peptide. The product was isolated by preparative RP HPLC (Column: SunFire C18 OBD™ Prep Column, 100 Å, 5 μm, 30 mm×100 mm; Eluent A: H₂O+0.1% TFA and eluent B: ACN). Pure fractions were combined and lyophilized to afford A52-2 (7.3 mg, 4.0 μmol, 4% yield) as a white solid. LCMS Method AP-1, t_{R}=5.97 min, [M+H]⁺=1655.7.

### Step 3. Example A52

A52-2 (7.3 mg, 1 eq., 4.4 μmol) was dissolved in DMF (2 mL) and pyrrolidine (25% in DMF) (31 mg, 36 μL, 25 eq., 110 μmol) was added. The reaction mixture was stirred at RT for 1.5 hr, then quenched with ACN/water (1/1). The quenched reaction mixture was directly purified by preparative R^{P} (Column: SunFire C18 OBD™ Prep Column, 100 Å, 5 μm, 30 mm×100 mm; Eluent A: H₂O+0.1% TFA and eluent B: ACN). Pure fractions were combined and lyophilized to afford Example A52 (3.3 mg, 2 μmol, 46% yield, TFA salt) as a white solid. LCMS Method AP-1, t_{R}=3.93 min, [M+H]⁺=1432.6.

### 2.1.9 Synthesis of (3R,6S,9S,12S,15S,18S,21S,24S,27S)-9-((1H-indol-3-yl)methyl)-27-amino-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-15,21-bis(hydroxymethyl)-N,4,16,19-tetramethyl-5,8,11,14,17,20,23,26,30-nonaoxo-18-phenethyl-1-thia-4,7,10,13,16,19,22,25,29-nonaazacyclohentriacontane-3-carboxamide TFA salt (Example A53)

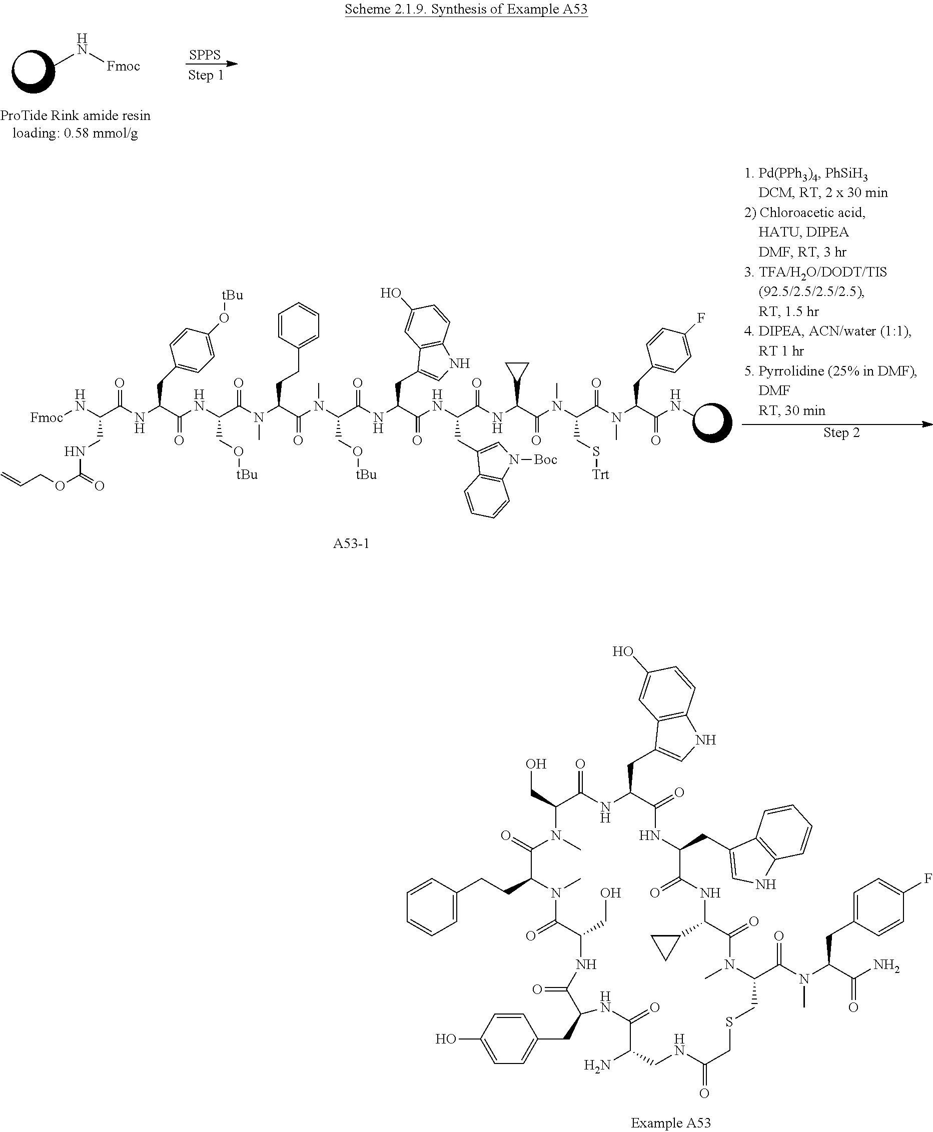

### Step 1. A53-1

The assembly of the linear peptide was done on the CEM Liberty Blue Synthesizer using Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×4 min at 90° C.
Method B Fmoc removal: 2×10 min at RT/Coupling: 2×4 min at 90° C.

After the assembly of the linear peptide the resin was washed with DMF (5×) and DCM (5×). The amino acids and coupling methods are summarized in Table 2.15.

TABLE 2.15

| Synthesis | Amino acid | | |
|---|---|---|---|
| cycle | residue^{a} | Amino acid | Method |
| 1 | A¹⁰ | Fmoc-L-N—Me-Phe(4F)—OH | A |
| 2 | A⁹ | Fmoc-L-N—Me-Cys(Trt)-OH | B |
| 3 | A⁸ | Fmoc-L-CyclopropylGly- | B |
| | | OH | |
| 4 | A⁷ | Fmoc-L-Trp(Boc)—OH | A |
| 5 | A⁶ | Fmoc-L-Trp(5-OH)—OH | A |
| 6 | A⁵ | Fmoc-L-N—Me-Ser(tBu)—OH | A |
| 7 | A⁴ | Fmoc-L-N—Me-Homo-Phe- | B |
| | | OH | |
| 8 | A³ | Fmoc-L-Ser(tBu)—OH | B |
| 9 | A² | Fmoc-L-Tyr(tBu)—OH | A |
| 10 | A¹ | Fmoc-L-Dap(Alloc)-OH | A |

^{a}Refers to the corresponding amino acid residue of cyclic peptide, P, as found, e.g., in Formulae (I), (Ia), (Ib), (Ic), (Id), or in the Table of Example III.

### Step 2. Example A53

Step 2-1: The resin from Step 1 (calculated with 100 μmol) was suspended with DCM (4 mL) and treated with phenylsilane (216 mg, 248 μL, 20 eq., 2 mmol). The suspension was shaken at RT for 5 min while purged with argon. Then Pd(PPh₃)₄ (11.6 mg, 0.1 eq., 10.0 μmol) was added at RT. The reaction was agitated with argon at RT for 30 min. The resin was drained, washed with DMF (3×) and DCM (3×), and the procedure was repeated once. The resin was drained and washed with DMF (5×) and DCM (5×).

Step 2-2: The resin from Step 2-1 was suspended with DMF (5 mL). Then chloroacetic acid (14.2 mg, 9 μL, 1.5 eq., 150 μmol), HATU (60.8 mg, 1.6 eq., 160 μmol) and DIPEA (51.7 mg, 69.7 μL, 4 eq., 400 μmol) were added. The reaction mixture was stirred at RT for 3 hr. The resin was drained and washed with DMF (5×) and DCM (5×).

Step 2-3: To the resin from Step 2-2 was added TFA/H₂O/DODT/TIS (92.5/2.5/2.5/2.5) (5 mL) and the resin was shaken at RT for 1.5 hr. The crude peptide was precipitated with cold diethyl ether (40 mL) giving a precipitate. The suspension was centrifuged, and the solvent was decanted. The product was washed with another portion of cold diethyl ether (30 mL). The suspension was centrifuged, and the solvent was decanted. The crude peptide was dried at RT under vacuo and purified by preparative RP HPLC (Column: XSelect® Peptide CSH C18, OBD™ Prep Column, 130 Å, 5 μm, 30×250 mm; Eluent A: H₂O+0.1% TFA and eluent B: ACN). The pure fraction was directly used for the next Step.

Step 2-4: The product fraction from Step 2-3 was diluted with ACN/water (1/1) (20 mL) and DIPEA (25.9 mg, 34.8 μL, 2 eq., 200 μmol) was added. The reaction mixture was stirred at RT for 1 hr, then directly lyophilized to afford the crude peptide.

Step 2-5: The crude peptide from Step 2-4 was dissolved in DMF (2 mL) and pyrrolidine (25% in DMF) (90 mg, 100 μL, 3 eq., 300 μmol) was added. The reaction mixture was stirred at RT for 30 min, then quenched with ACN/water (1/1). The quenched reaction mixture was directly purified by preparative R^{P} (Column: XSelect® Peptide CSH C18, OBD™ Prep Column, 130 Å, 5 μm, 30×250 mm; Eluent A: H₂O+0.1% TFA and eluent B: ACN). Pure fractions were combined and lyophilized to afford Example A53 (2.3 mg, 1.4 μmol, 1% yield, TFA salt) as a white solid. LCMS Method AP-1, t_{R}=3.84 min, [M+2H]²⁺=726.3.

### 2.1.10 Synthesis of (4R,7S,10S,13S,16S,19S,22S,25S,28R)-10-((1H-indol-3-yl)methyl)-28-amino-N—((S)-1-amino-3-(4-fluorophenyl)-1-oxopropan-2-yl)-7-cyclopropyl-13-((5-hydroxy-1H-indol-3-yl)methyl)-25-(4-hydroxybenzyl)-16,22-bis(hydroxymethyl)-N,5,17,20-tetramethyl-6,9,12,15,18,21,24,27-octaoxo-19-phenethyl-1,2-dithia-5,8,11,14,17,20,23,26-octaazacyclononacosane-4-carboxamide TFA salt (Example A54 (A54-1 disclosed as SEQ ID NO: 113 and A54 disclosed as SEQ ID NO: 19))

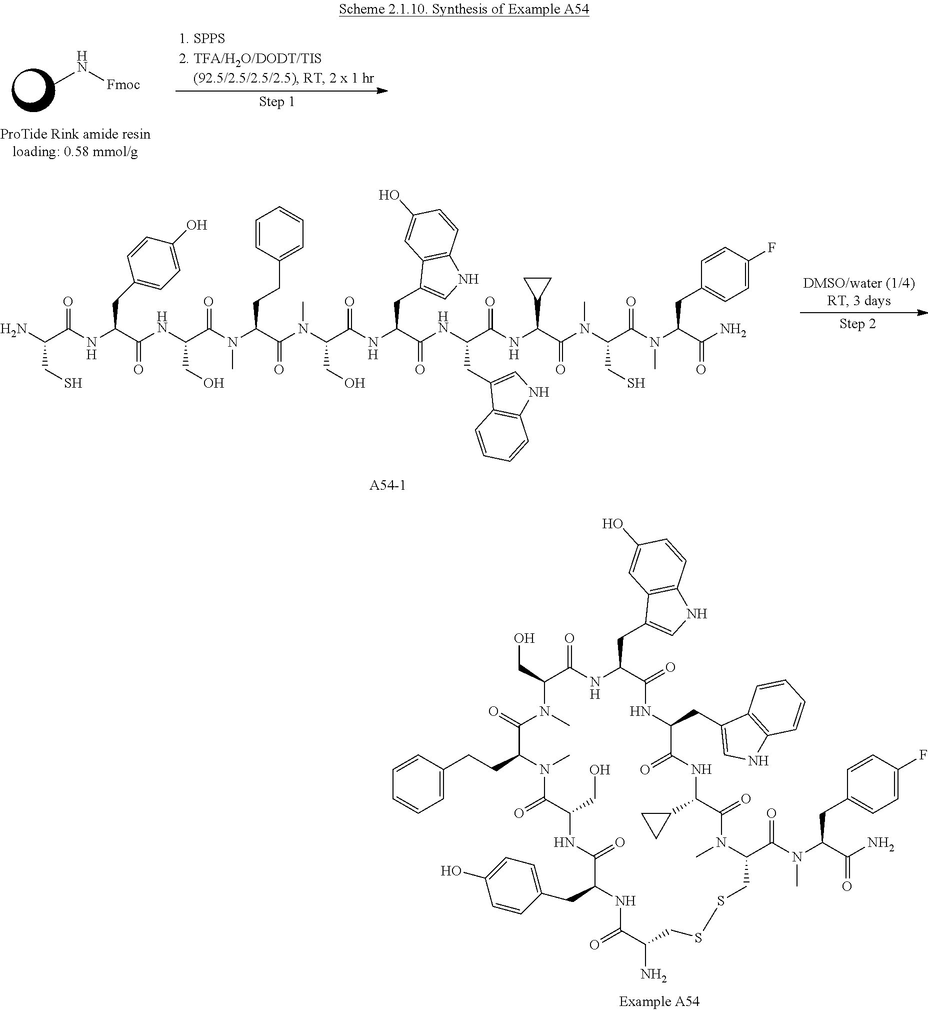

### Step 1. A54-1

Step 1-1: The assembly of the linear peptide was done on the CEM Liberty Prime Synthesizer using commercial Rink amide resin R1 (loading 0.58 mmol/g, 172 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.
Method C Fmoc removal: 1×40 sec at 110° C./Coupling: 2×1 min at 105° C.
Method D Fmoc removal: 2×10 min at RT/Coupling: 1×2 min at 105° C.

After the assembly of the linear peptide the resin was washed with DMF (5×) and DCM (5×). The amino acids and coupling methods are summarized in Table 2.16.

TABLE 2.16

| Synthesis | Amino acid | | |
|---|---|---|---|
| cycle | residue^{a} | Amino acid | Method |
| 1 | A¹⁰ | Fmoc-L-N—Me-Phe(4F)—OH | A |
| 2 | A⁹ | Fmoc-L-N—Me-Cys(Trt)-OH | B |
| 3 | A⁸ | Fmoc-L-CyclopropylGly- | B |
| | | OH | |
| 4 | A⁷ | Fmoc-L-Trp(Boc)—OH | D |
| 5 | A⁶ | Fmoc-L-Trp(5-OH)—OH | C |
| 6 | A⁵ | Fmoc-L-N—Me-Ser(tBu)—OH | C |
| 7 | A⁴ | Fmoc-L-N—Me-Homo-Phe- | B |
| | | OH | |
| 8 | A³ | Fmoc-L-Ser(tBu)—OH | B |
| 9 | A² | Fmoc-L-Tyr(tBu)—OH | D |
| 10 | A¹ | Fmoc-L-Cys(Trt)-OH | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (5 mL) and the resin was shaken at RT for 1 hr. The crude peptide was precipitated with cold diethyl ether (35 mL). The suspension was centrifuged, and the solvent was decanted. The product was washed with another portion of cold diethyl ether (30 mL). The suspension was centrifuged, and the solvent was decanted. The cleavage procedure was repeated once. The combined crude peptide was dried at RT under vacuo, and the product was isolated by preparative RP HPLC (Column: SunFire C18 OBD™ Prep Column, 100 Å, 5 μm, 30 mm×100 mm; Eluent A: H₂O+0.1% TFA and eluent B: ACN). Pure fractions were combined and lyophilized to afford A54-1 (20.1 mg, 14.1 μmol, 20% yield, TFA salt) as a white solid. LCMS Method AP-1, t_{R}=4.01 min, [M+H]⁺=1428.6.

### Step 2. Example A54

A54-1 (20.1 mg, 1 eq., 14.1 μmol TFA salt) was dissolved in DMSO/water (1/4) (7.5 mL). The reaction mixture was stirred at RT for 3 days. The reaction mixture was lyophilized to afford the crude peptide and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A54 (6.2 mg, 3.7 μmol, 26% yield, TFA salt) as a white solid. LCMS Method AP-1, t_{R}=3.89 min, [M+H]⁺=1426.6.

The compounds in Table 2.17 were synthesized in analogy to Example A54.

TABLE 2.17
Examples synthesized in analogy to Example A54 (Scheme 2.1.10).

| Ex. No. | Structure/Example Name | LCMS |
|---|---|---|
| A55 | 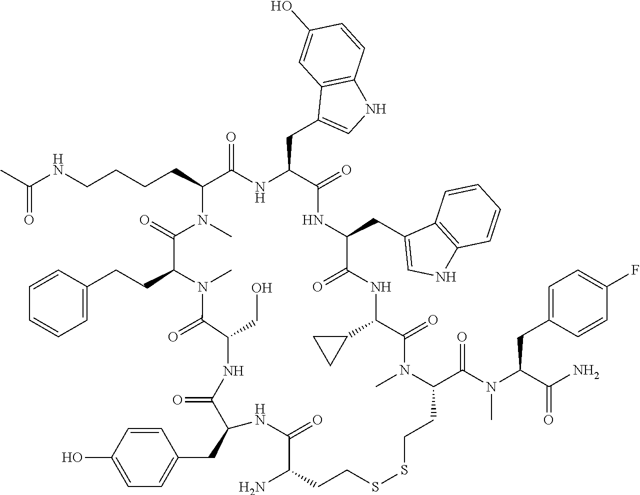 | Method AP-1 t_{R} = 4.02 min [M + H]⁺ = 1537.7 |
| | (5S,8S,11S,14S,17S,20S,23S,26S,29S)-11-((1H-indol-3-yl)methyl)-17-(4- | |
| | acetamidobutyl)-29-amino-N-((S)-1-amino-3-(4-fluorophenyl)-1-oxopropan-2- | |
| | y1)-8-cyclopropyl-14-((5-hydroxy-1H-indol-3-yl)methyl)-26-(4- | |
| | hydroxybenzyl)-23-(hydroxymethyl)-N,6,18,21-tetramethyl- | |
| | 7,10,13,16,19,22,25,28-octaoxo-20-phenethyl-1,2-dithia- | |
| | 6,9,12,15,18,21,24,27-octaazacyclohentriacontane-5-carboxamide TFA salt | |
| A56 (SEQ ID NO: 20) | 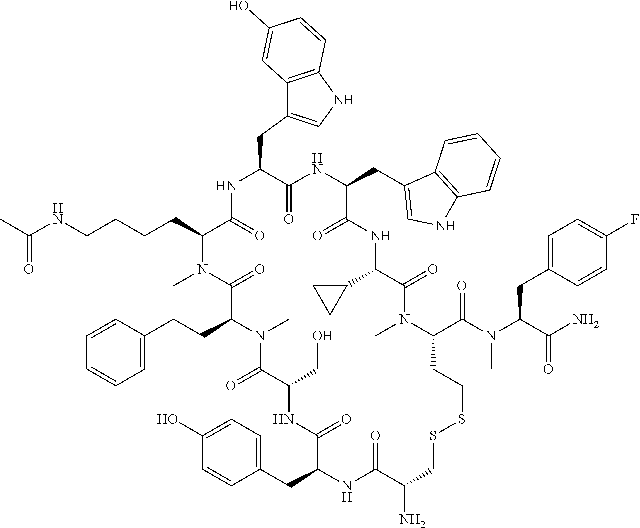 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(N)=O)CCSSC[C@H](N)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 3.99 min [M + H]⁺ = 1523.7 |
| | (5S,8S,11S,14S,17S,20S,23S,26S,29R)-11-((1H-indol-3-yl)methyl)-17-(4- | |
| | acetamidobutyl)-29-amino-N-((S)-1-amino-3-(4-fluorophenyl)-1-oxopropan-2- | |
| | yl)-8-cyclopropyl-14-((5-hydroxy-1/-indol-3-y1)methyl)-26-(4- | |
| | hydroxybenzyl)-23-(hydroxymethyl)-N,6,18,21-tetramethyl- | |
| | 7,10,13,16,19,22,25,28-octaoxo-20-phenethyl-1,2-dithia- | |
| | 6,9,12,15,18,21,24,27-octaazacyclotriacontane-5-carboxamide TFA salt | |
| A57 | 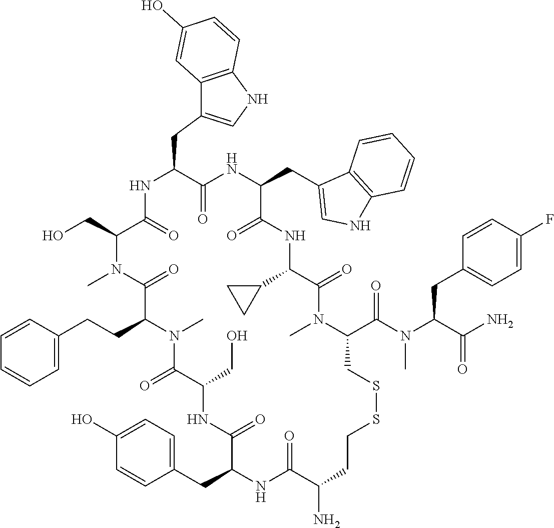 [SMILES: CN1C(=O)[C@H](CO)NC(=O)[C@H](Cc2ccc(O)cc2)NC(=O)[C@@H](N)CCSSC[C@@H](C(=O)N(C)[C@@H](Cc2ccc(F)cc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CO)N(C)C(=O)[C@@H]1CCc1ccccc1] | Method AP-1 t_{R} = 4.00 min [M + H]⁺ = 1440.6 |
| | (4R,7S,10S,13S,16S,19S,22S,25S,28S)-10-((1H-indol-3-yl)methyl)-28-amino- | |
| | N-((S)-1-amino-3-(4-fluorophenyl)-1-oxopropan-2-y1)-7-cyclopropyl-13-((5- | |
| | hydroxy-1H-indol-3-y1)methyl)-25-(4-hydroxybenzyl)-16,22- | |
| | bis(hydroxymethyl)-N,5,17,20-tetramethyl-6,9,12,15,18,21,24,27-octaoxo-19- | |
| | phenethyl-1,2-dithia-5,8,11,14,17,20,23,26-octaazacyclotriacontane-4- | |
| | carboxamide TFA salt | |

### 2.1.11 Synthesis of (3S,6S,9S,12S,15S,18S,21S,24S,27S)-9-((1H-indol-3-yl)methyl)-15-(4-acetamidobutyl)-27-amino-N—((S)-1-amino-3-(4-fluorophenyl)-1-oxopropan-2-yl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide 5 TFA salt (Example A58) and (3S,6S,9S,12S,15S,18S,21S,24S,27S)-9-((1H-indol-3-yl)methyl)-15-(4-acetamidobutyl)-N—((S)-1-amino-3-(4-fluorophenyl)-1-oxopropan-2-yl)-27-(1-amino-3,6,9,12,15,18,21,24,27,30-decaoxatritriacontan-33-amido)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide TFA salt (Example A59)

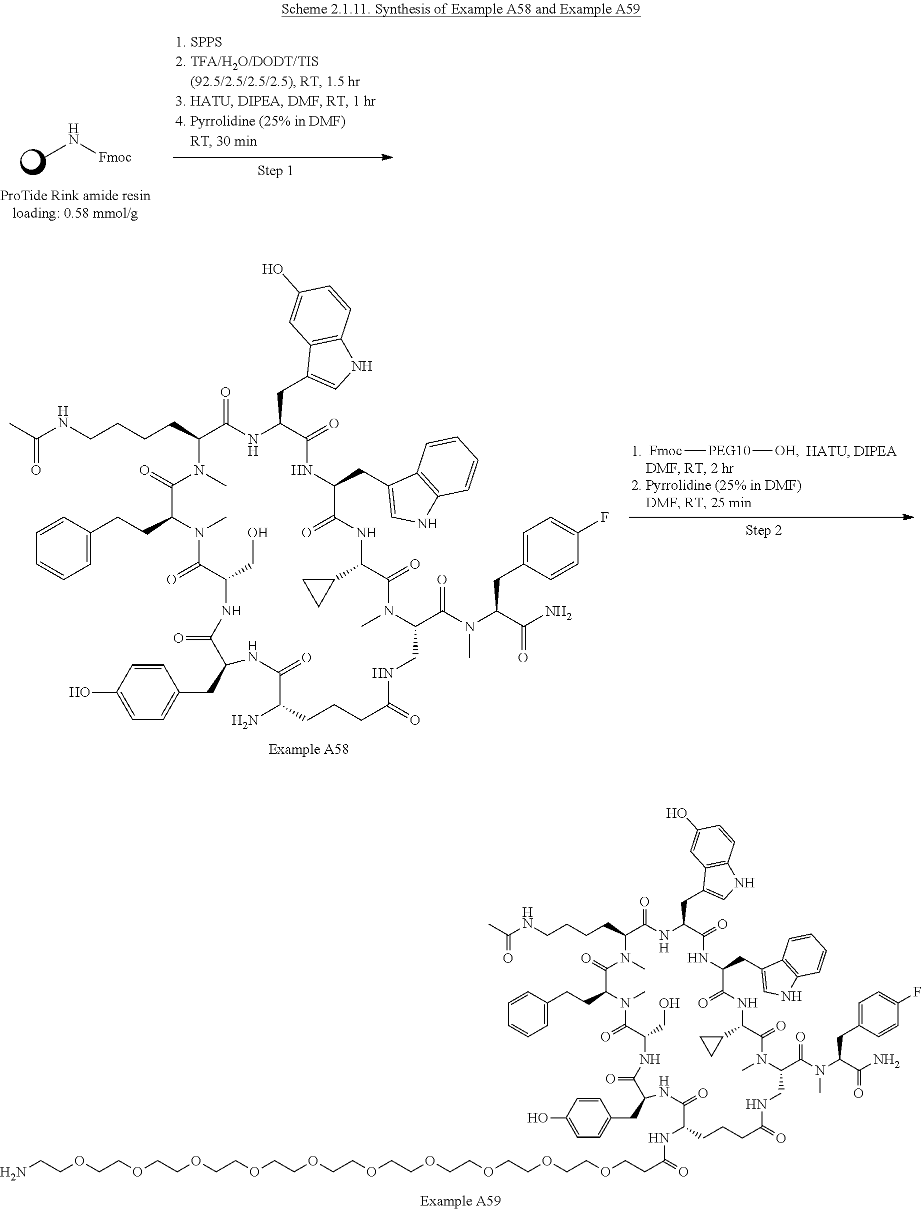

### Step 1. Example A58

Step 1-1: The assembly of the linear peptide was done on the CEM Liberty Prime Synthesizer and by manual coupling using commercial Rink amide resin R1 (loading 0.58 mmol/g, 172 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.

Fmoc deprotection (after Synthesis cycle 5) was performed using 25% pyrrolidine in DMF (0.75 mL), addition by synthesizer, 1×40 sec at 110° C.

### Method C (Manual Coupling)

The resin was treated with a pre-activated solution of Fmoc-L-N-Me-Lys (Ac)—OH (50.9 mg, 1.2 eq., 120 μmol), HATU (57.0 mg, 1.5 eq., 150 μmol) and DIPEA (38.8 mg, 52.3 μL, 3 eq., 300 μmol) in DMF (4 mL) and mixed at RT for 5 hr.

After the assembly of the linear peptide the resin was washed with DMF (5×) and DCM (5×). The amino acids and coupling methods are summarized in Table 2.18.

TABLE 2.18

| Synthesis | Amino acid | | |
|---|---|---|---|
| cycle | residue^{a} | Amino acid | Method |
| 1 | A¹⁰ | Fmoc-L-N—Me-Phe(4F)—OH | A |
| 2 | A⁹ | Fmoc-L-N—Me-Dap(Boc)—OH | B |
| 3 | A⁸ | Fmoc-L-CyclopropylGly-OH | B |
| 4 | A⁷ | Fmoc-L-Trp(Boc)—OH | A |
| 5 | A⁶ | Fmoc-L-Trp(5-OH)—OH | A + Fmoc |
| | | | deprotection |
| 6 | A⁵ | Fmoc-L-N—Me-Lys(Ac)—OH | C |
| | | | (Manual |
| | | | coupling) |
| 7 | A⁴ | Fmoc-L-N—Me-Homo-Phe-OH | B |
| 8 | A³ | Fmoc-L-Ser(tBu)—OH | B |
| 9 | A² | Fmoc-L-Tyr(tBu)—OH | A |
| 10 | A¹ | Fmoc-L-Homo-Glu(OtBu)—OH | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (3 mL) and the resin was shaken at RT for 1.5 hr. The crude peptide was precipitated with cold diethyl ether (30 mL). The suspension was centrifuged, and the solvent was decanted. The product was washed with another portion of cold diethyl ether (20 mL). The suspension was centrifuged, and the solvent was decanted. The crude peptide was dried at RT under vacuo.

Step 1-3: The crude peptide from Step 1-2 (calculated with 100 μmol) was dissolved in DMF (3 mL). Then HATU (45.6 mg, 1.2 eq., 120 μmol), followed by DIPEA (51.7 mg, 69.7 μL, 4 eq., 400 μmol) were added. The reaction mixture was stirred at RT for 1 hr.

Step 1-4: The reaction mixture from Step 1-3 was directly treated with pyrrolidine (25% in DMF) (427 mg, 493 μL, 15 eq., 1500 μmol) and stirred at RT for 30 min, then quenched with ACN/water (1/1). The quenched reaction mixture was directly purified by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A58 (15.4 mg, 6.6 μmol, 6.6% yield, TFA salt) as a white solid. LCMS Method AP-1, t_{R}=3.85 min, [M+H]⁺=1516.7.

### Step 2. Example A59

Step 2-1 To a clear solution of Example A58 (15.4 mg, 1 eq., 9.44 μmol, TFA salt) and Fmoc-PEG10-OH (8.52 mg, 1.2 eq., 11.33 μmol) in DMF (3 mL) was added HATU (5.4 mg, 1.5 eq., 14.17 μmol) and DIPEA (4.88 mg, 6.6 μL, 4 eq., 37.77 μmol). The reaction mixture was stirred at RT for 2 hr.

Step 2-2: The reaction mixture from Step 2-1 was directly treated with pyrrolidine (25% solution in DMF) (62 μL, 20 eq., 188.9 μmol) and stirred at RT for 25 min. The reaction mixture was quenched with ACN/water (1/1). The quenched reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A59 (6 mg, 2.4 μmol, 26% yield, TFA salt) as a white solid. LCMS Method AP-1, t_{R}=4.14 min, [M+H]⁺=2029.0.

The following examples (for structures, see Table 2.19) were synthesized in analogy to Step 2 of Example A59:

Example A76 was synthesized from Example A40.

Example A77 was synthesized from Example A45.

TABLE 2.19
Examples synthesized in analogy to Step 2 of Example A59 (Scheme 2.1.11).

| Ex. No. | Structure/Example Name | LCMS |
|---|---|---|
| A76 (SEQ ID NO: 34) | 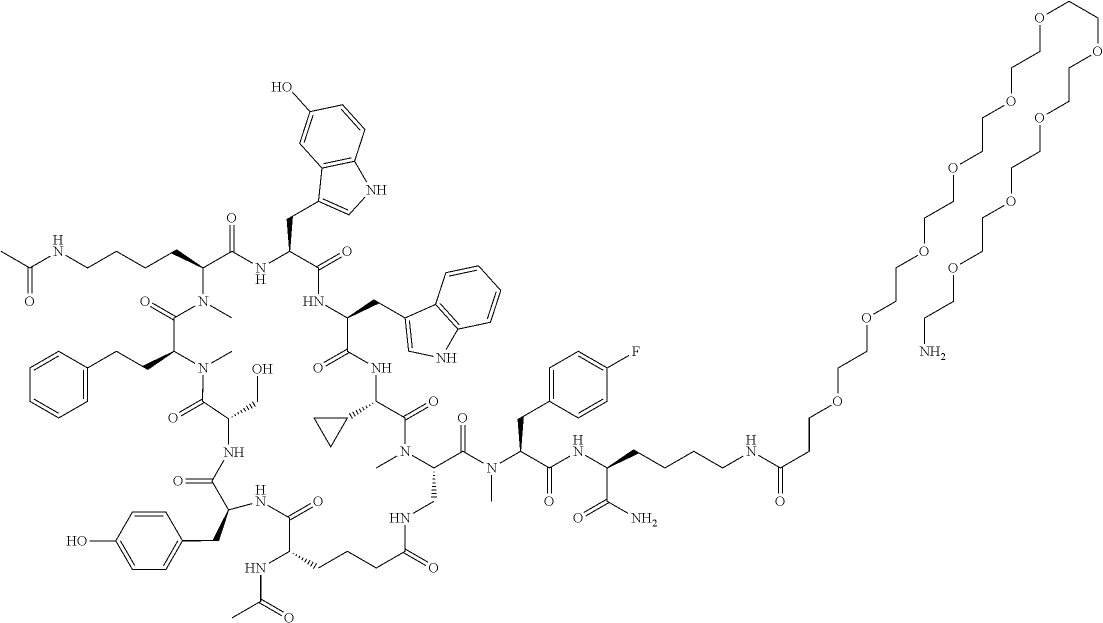 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CCOCCOCCOCCOCCOCCOCCOCCOCCOCCOCCN)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 4.07 min [M + H]⁺ = 2199.2 |
| | (3S,6S,9S,12S,15S,18S,21S,24S,27S)-9-((1H-indol-3-yl)methyl)-27-acetamido-15-(4-acetamidobutyl)-N- | |
| | ((39S,42S)-1-amino-39-carbamoyl-43-(4-fluorophenyl)-33,41-dioxo-3,6,9,12,15,18,21,24,27,30-decaoxa- | |
| | 34,40-diazatritetracontan-42-y1)-6-cyclopropyl-12-((5-hydroxy-1H-indol-3-yl)methyl)-24-(4-hydroxybenzyl)- | |
| | 21-(hydroxymethyl)-N,4,16,19-tetramethyl-5,8,11,14,17,20,23,26,31-nonaoxo-18-phenethyl- | |
| | 1,4,7,10,13,16,19,22,25-nonaazacyclohentriacontane-3-carboxamide TFA salt | |
| A77 (SEQ ID NO: 35) | 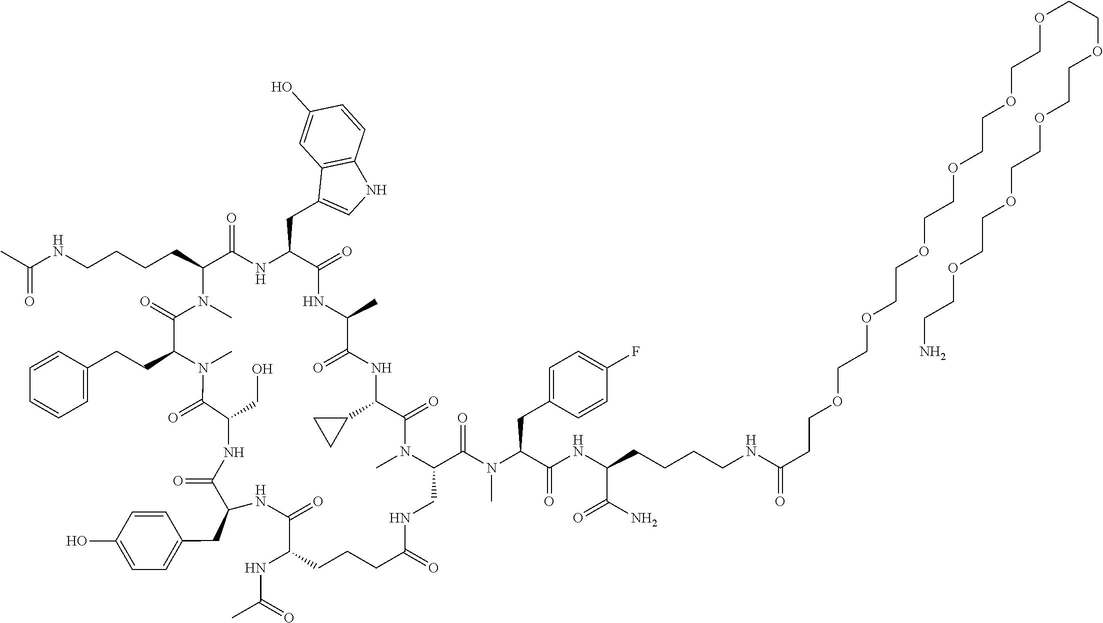 | Method AP-1 t_{R} = 3.75 min [M + H]⁺ = 2084.1 |
| | (3S,6S,9S,12S,15S,18S,21S,24S,27S)-27-acetamido-15-(4-acetamidobutyl)-N-((39S,42S)-1-amino-39- | |
| | carbamoyl-43-(4-fluorophenyl)-33,41-dioxo-3,6,9,12,15,18,21,24,27,30-decaoxa-34,40-diazatritetracontan-42- | |
| | y1)-6-cyclopropyl-12-((5-hydroxy-1/-indol-3-yl)methyl)-24-(4-hydroxybenzyl)-21-(hydroxymethyl)- | |
| | N,4,9,16,19-pentamethyl-5,8,11,14,17,20,23,26,31-nonaoxo-18-phenethyl-1,4,7,10,13,16,19,22,25- | |
| | nonaazacyclohentriacontane-3-carboxamide TFA salt | |

### 2.1.12 Synthesis of 3-(((3S,6S,9S,12S,15S,18S,21S,24S,27S)-9-((1H-indol-3-yl)methyl)-15-(4-acetamidobutyl)-6-cyclopropyl-3-(((S)-1-(((S)-1,6-diamino-1-oxohexan-2-yl)amino)-3-(4-fluorophenyl)-1-oxopropan-2-yl)(methyl)carbamoyl)-12-((5-hydroxy-1H-indol-3-yl)methyl)-24-(4-hydroxybenzyl)-21-(hydroxymethyl)-4,16,19-trimethyl-5,8,11,14,17,20,23,26,31-nonaoxo-18-phenethyl-1,4,7,10,13,16,19,22,25-nonaazacyclohentriacontan-27-yl)amino)-3-oxopropanoic acid TFA salt (Example A60 (A60 disclosed as SEQ ID NO: 21))

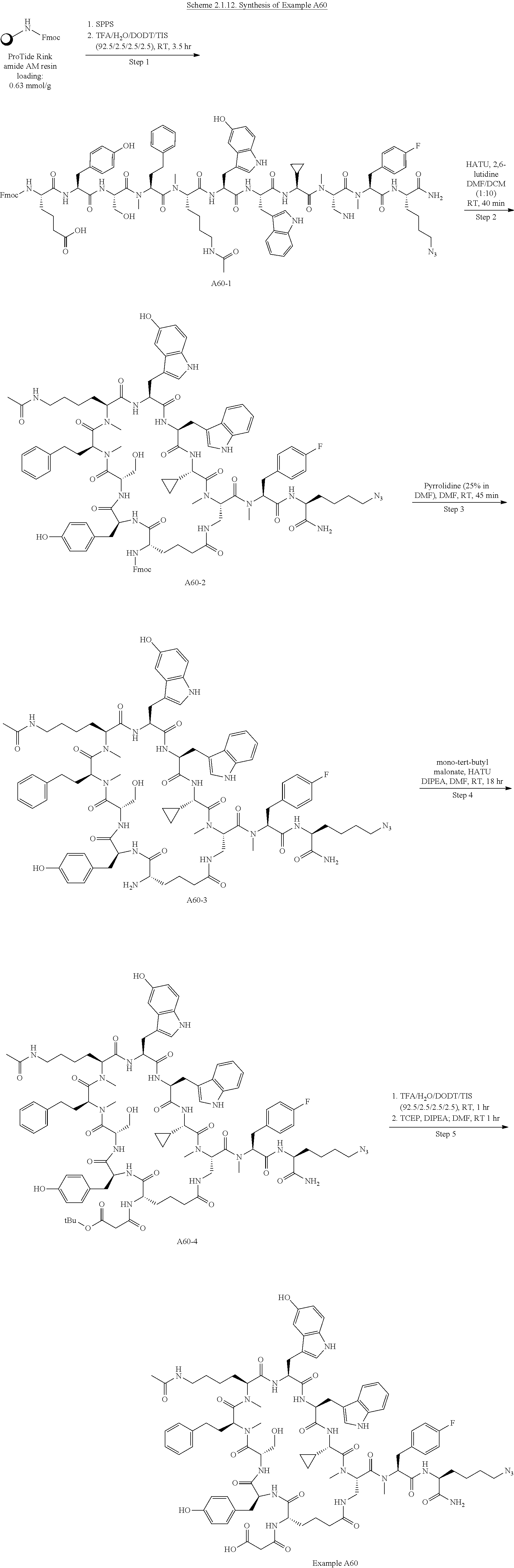

### Step 1. A60-1

Step 1-1: The assembly of the linear peptide was done on the CEM Liberty Prime Synthesizer and by manual coupling using commercial Rink amide AM resin R2 (loading 0.63 mmol/g, 1.587 g, 1.000 mmol, SPPS performed in 2 batches of 0.500 mmol scale). The couplings were carried out using the following solutions: Fmoc-amino acid (0.5 M solution DMF, 4 mL, 4 eq.), DIC (4 M solution in DMF, 1 mL, 8 eq.), Oxyma Pure® (0.25 M solution in DMF, 8 mL, 4 eq.), addition by synthesizer. Fmoc removal was performed using a solution of pyrrolidine (25% in DMF, 2.5 mL), addition by synthesizer.

The amino acids were coupled with a set of different methods.
Method A Fmoc removal: 1×1 min at 110° C./Coupling: 1×4 min at 105° C.
Method B Fmoc removal: 2×10 min at RT/Coupling: 2×4 min at 105° C.

Fmoc deprotection (after Synthesis cycle 10) was performed using 25% pyrrolidine in DMF (2.5 mL), addition by synthesizer, 1×1 min at 110° C.

### Method C (Manual Coupling)

The resin was treated with a pre-activated solution of Fmoc-L-Homo-Glu(OtBu)-OH (659.3 mg, 1.5 eq., 1.5 mmol), HATU (608.4 mg, 1.6 eq., 1.6 mmol) and DIPEA (646.3 mg, 871 μL, 5 eq., 5.0 mmol) in DMF (20 mL) and mixed at RT for 2 hr.

After the assembly of the linear peptide the resin was washed with DMF (5×) and DCM (5×). The amino acids and coupling methods are summarized in Table 2.20.

TABLE 2.20

| Synthesis | Amino acid | | |
|---|---|---|---|
| cycle | residue^{a} | Amino acid | Method |
| 1 | A¹⁰* | Fmoc-L-Lys(N3)—OH | A |
| 2 | A¹⁰ | Fmoc-L-N—Me-Phe(4F)—OH | A |
| 3 | A⁹ | Fmoc-L-N—Me-Dap(Boc)—OH | B |
| 4 | A⁸ | Fmoc-L-CyclopropylGly-OH | B |
| 5 | A⁷ | Fmoc-L-Trp(Boc)—OH | B |
| 6 | A⁶ | Fmoc-L-Trp(5-OH)—OH | A |
| 7 | A⁵ | Fmoc-L-N—Me-Lys(Ac)—OH | A |
| 8 | A⁴ | Fmoc-L-N—Me-Homo-Phe-OH | B |
| 9 | A³ | Fmoc-L-Ser(tBu)—OH | B |
| 10 | A² | Fmoc-L-Tyr(tBu)—OH | B + Fmoc |
| | | | deprotection |
| 11 | A¹ | Fmoc-L-Homo-Glu(OtBu)—OH | C |
| | | | (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (40 mL) and the resin was shaken at RT for 3.5 hr. The crude peptide was precipitated with cold heptane/MTBE (1/1) (450 mL). The suspension was centrifuged, and the solvent was decanted. The product was washed with another portion of cold heptane/MTBE (400 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.08% NH₄HCO₃ and eluent B: ACN). Pure fractions were combined and lyophilized to afford A60-1 (465 mg, 0.24 mmol, 24% yield, TFA salt) as a white solid. LCMS Method AP-1, t_{R}=4.98 min, [M+H]=1911.7.

### Step 2. A60-2

To a solution of A60-1 (465 mg, 1 eq., 0.243 mmol, TFA salt) in DMF/DCM (1/10) (320 mL) at RT was added 2,6-lutidine (651.8 mg, 705 μL, 25 eq., 6.083 mmol). A solution of HATU (101.8 mg, 1.1 eq., 0.267 mmol) in DMF (1 mL) was then added dropwise. The reaction mixture was stirred at RT for 40 min and then concentrated at 35° C. in vacuo to remove the DCM and partially the DMF. The residue was precipitated with cold MTBE (100 mL). The suspension was centrifuged, and the solvent was decanted. The product was washed with another portion of cold MTBE (40 mL). The suspension was centrifuged, and the solvent was decanted to afford the crude peptide. The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A60-2 (298 mg, 0.16 mmol, 64% yield) as a white solid. LCMS Method AP-1, t_{R}=5.97 min, [M+H]=1893.9.

### Step 3. A60-3

To a solution of A60-2 (101.0 mg, 1 eq., 53.35 μmol) in DMF (2 mL) was added pyrrolidine (25% solution in DMF) (151.8 mg, 175 μL, 10 eq., 533.5 μmol) and the reaction mixture was stirred at RT for 20 min. The reaction mixture was precipitated with cold MTBE (45 mL) giving a precipitate. The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A60-3 (85.2 mg, 47 μmol, 89% yield, TFA salt) as a white solid. LCMS Method AP-1, t_{R}=4.21 min, [M+H]=1670.8.

### Step 4. A60-4

To a clear solution of A60-3 (85.2 mg, 1 eq., 47.73 μmol, TFA salt) at RT was added a solution of mono-tert-butyl malonate (CAS 40052-13-9, 11.47 mg, 11.0 μL, 1.5 eq., 71.60 μmol), HATU (23.59 mg, 1.3 eq., 62.05 μmol) and DIPEA (30.85 mg, 41.6 μL, 5 eq., 238.7 μmol) in DMF (0.5 mL) (pre-activated at RT for 5 min). The reaction mixture was stirred at RT for 16 hr. A solution of mono-tert-butyl malonate (CAS 40052-13-9, 114.7 mg, 110 μL, 15 eq., 716 μmol), HATU (235.9 mg, 13 eq., 620.5 μmol) and DIPEA (123.4 mg, 166 μL, 20 eq., 954.7 μmol) in DMF (1.0 mL) (pre-activated at RT for 5 min) was then added and the reaction mixture was stirred at RT for another 2 hr. The reaction mixture was precipitated with cold MTBE (45 mL) giving a precipitate. The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A60-4 (40 mg, 22 μmol, 46% yield) as a white solid. LCMS Method AP-1, t_{R}=5.41 min, [M+H]=1813.9.

### Step 5. Example A60

Step 5-1: A60-4 (40.0 mg, 1 eq., 22.06 μmol) was dissolved in TFA/H₂O/DODT/TIS (92.5/2.5/2.5/2.5) (1 mL) and stirred at RT for 1 hr. The reaction mixture was precipitated with cold heptane/MTBE (1/1) (45 mL). The suspension was centrifuged, and the solvent was decanted. The product was washed with another portion of cold heptane/MTBE (40 mL). The suspension was centrifuged, and the solvent was decanted. The crude peptide was dried at RT under vacuo.

Step 5-2: The crude peptide from Step 5-1 was dissolved in DMF (1.00 mL), then TCEP (25.3 mg, 4 eq., 88.25 μmol, HCl salt) and DIPEA (17.11 mg, 23.1 μL, 6 eq., 132.4 μmol) were added. The reaction mixture was shaken at RT for 2 hr. The reaction mixture was precipitated with cold heptane/MTBE (1/1) (45 mL). The suspension was centrifuged, and the solvent was decanted. The product was washed with another portion of cold heptane/MTBE (40 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A60 (16 mg, 8.5 μmol, 39% yield, TFA salt) as a white solid. LCMS Method AP-1, t_{R}=3.91 min, [M+H]⁺=1731.8.

### 2.2 Examples Synthesized Using Lys-Wang Resin R4

### 2.2.1 Synthesis of ((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl) propanoyl)-L-lysine TFA Salt (Example A61 (A61 disclosed as SEQ ID NO: 22))

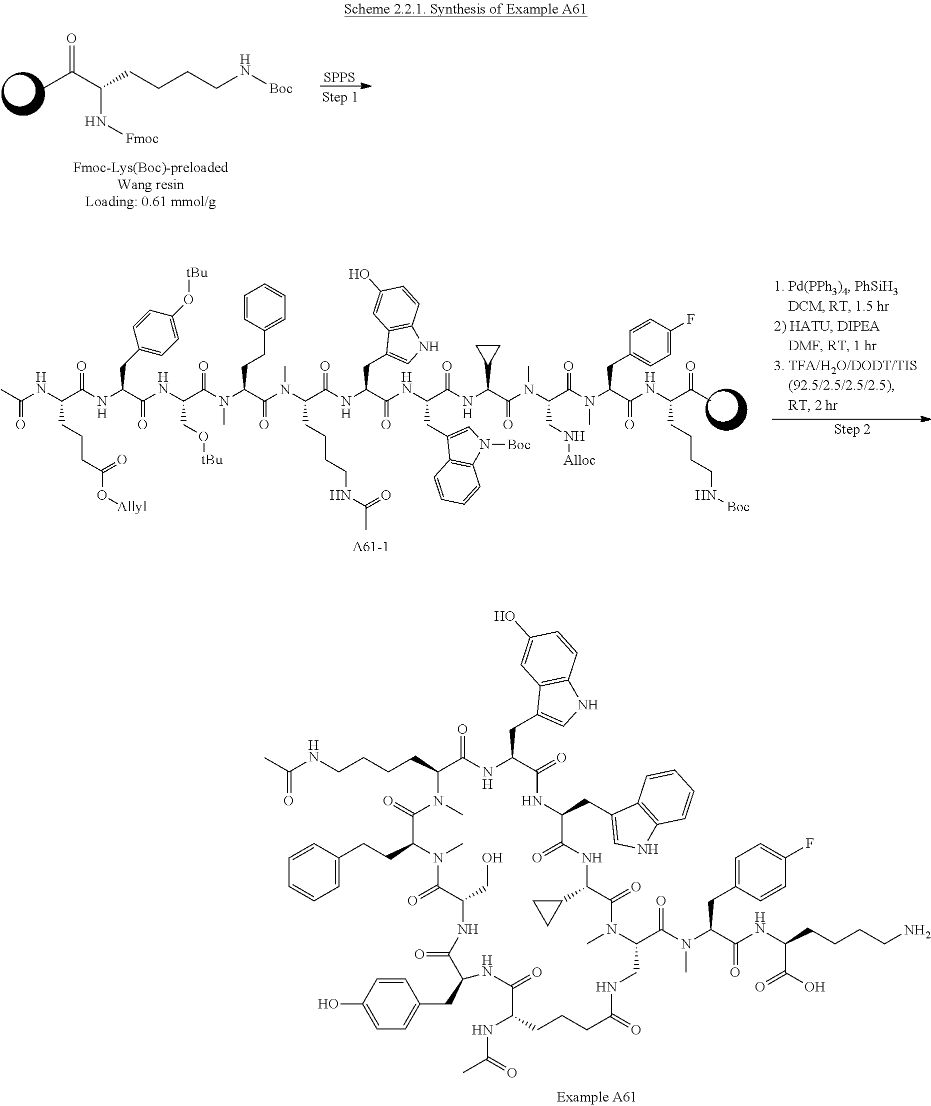

### Step 1. A61-1

The assembly of the linear peptide was done on the CEM Liberty Prime Synthesizer and by manual coupling using commercial Fmoc-Lys (Boc) preloaded Wang resin R4 (loading 0.61 mmol/g, 164 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.
Method C Fmoc removal: 2×10 min at RT/Coupling: 1×2 min at 105° C.

Fmoc deprotection (after Synthesis cycle 3) was performed using 25% pyrrolidine in DMF (0.75 mL), addition by synthesizer, 1×40 sec at 110° C.

### Method D (Manual Coupling)

The resin was treated with a pre-activated solution of Fmoc-L-CyclopropylGly-OH (37.1 mg, 1.1 eq., 110 μmol), HATU (57.0 mg, 1.5 eq., 150 μmol) and DIPEA (38.8 mg, 52.3 μL, 3 eq., 300 μmol) in DMF (4 mL) and mixed at RT for 4 hr.

After the assembly of the linear peptide the resin was washed with DMF (5×) and DCM (5×). The amino acids and coupling methods are summarized in Table 2.21.

TABLE 2.21

| Synthesis | Amino acid | | |
|---|---|---|---|
| cycle | residue^{a} | Amino acid | Method |
| 1 | A¹⁰* | Fmoc-L-Lys(Boc)—OH | Pre-loaded |
| | | | on resin |
| 2 | A¹⁰ | Fmoc-L-N—Me-Phe(4F)—OH | A |
| 3 | A⁹ | Fmoc-L-N—Me-Dap(Alloc)-OH | B + Fmoc |
| | | | deprotection |
| 4 | A⁸ | Fmoc-L-CyclopropylGly-OH | D |
| | | | (Manual |
| | | | coupling) |
| 5 | A⁷ | Fmoc-L-Trp(Boc)—OH | A |
| 6 | A⁶ | Fmoc-L-Trp(5-OH)—OH | A |
| 7 | A⁵ | Fmoc-L-N—Me-Lys(Ac)—OH | A |
| 8 | A⁴ | Fmoc-L-N—Me-Homo-Phe-OH | B |
| 9 | A³ | Fmoc-L-Ser(tBu)—OH | B |
| 10 | A² | Fmoc-L-Tyr(tBu)—OH | C |
| 11 | A¹ | Fmoc-L-Homo-Glu(OAll)-OH | A |
| 12 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

### Step 2. A61

Step 2-1: The resin from Step 1 (calculated with 100 μmol) was suspended with DCM (5 mL) and treated with phenylsilane (216 mg, 248 μL, 20 eq., 2 mmol). The suspension was shaken at RT for 5 min while purged with argon. Then Pd(PPh₃)+ (23.1 mg, 0.2 eq., 20.0 μmol) was added at RT. The reaction was agitated with argon at RT for 1.5 hr. The resin was drained and washed with DMF (3×) and DCM (5×).

Step 2-2: The resin from Step 2-1 (calculated with 100 μmol) was suspended with DMF (10 mL). Then HATU (57 mg, 1.5 eq., 150 μmol), followed by DIPEA (38.8 mg, 52.3 μL, 3 eq., 300 μmol) were added. The reaction mixture was stirred at RT for 1 hr. The resin was drained and washed with DMF (3×) and DCM (5×).

Step 2-3: To the resin from Step 2-2 (calculated with 100 μmol) was added TFA/H₂O/DODT/TIS (92.5/2.5/2.5/2.5) (4 mL) and the resin was shaken at RT for 2 hr. The crude peptide was precipitated with cold diethyl ether (40 mL) giving a precipitate. The suspension was centrifuged, and the solvent was decanted. The product was washed with another portion of cold diethyl ether (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A61 (10 mg, 2.4 μmol, 2.4% yield, TFA salt) as a white solid. LCMS Method AP-1, t_{R}=3.84 min, [M+H]=1687.8.

### 2.3 Examples Synthesized Using Sieber Rink Amide Resin R3

### 2.3.1 Synthesis of (3S,6S,9S,12S,15S,18S,21S,24S)-9-((1H-indol-3-yl)methyl)-15-(4-acetamidobutyl)-6-cyclopropyl-N—((S)-1-(((S)-1,6-diamino-1-oxohexan-2-yl)amino)-3-(4-fluorophenyl)-1-oxopropan-2-yl)-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide TFA salt (Example A62 (A62 disclosed as SEQ ID NO: 23))

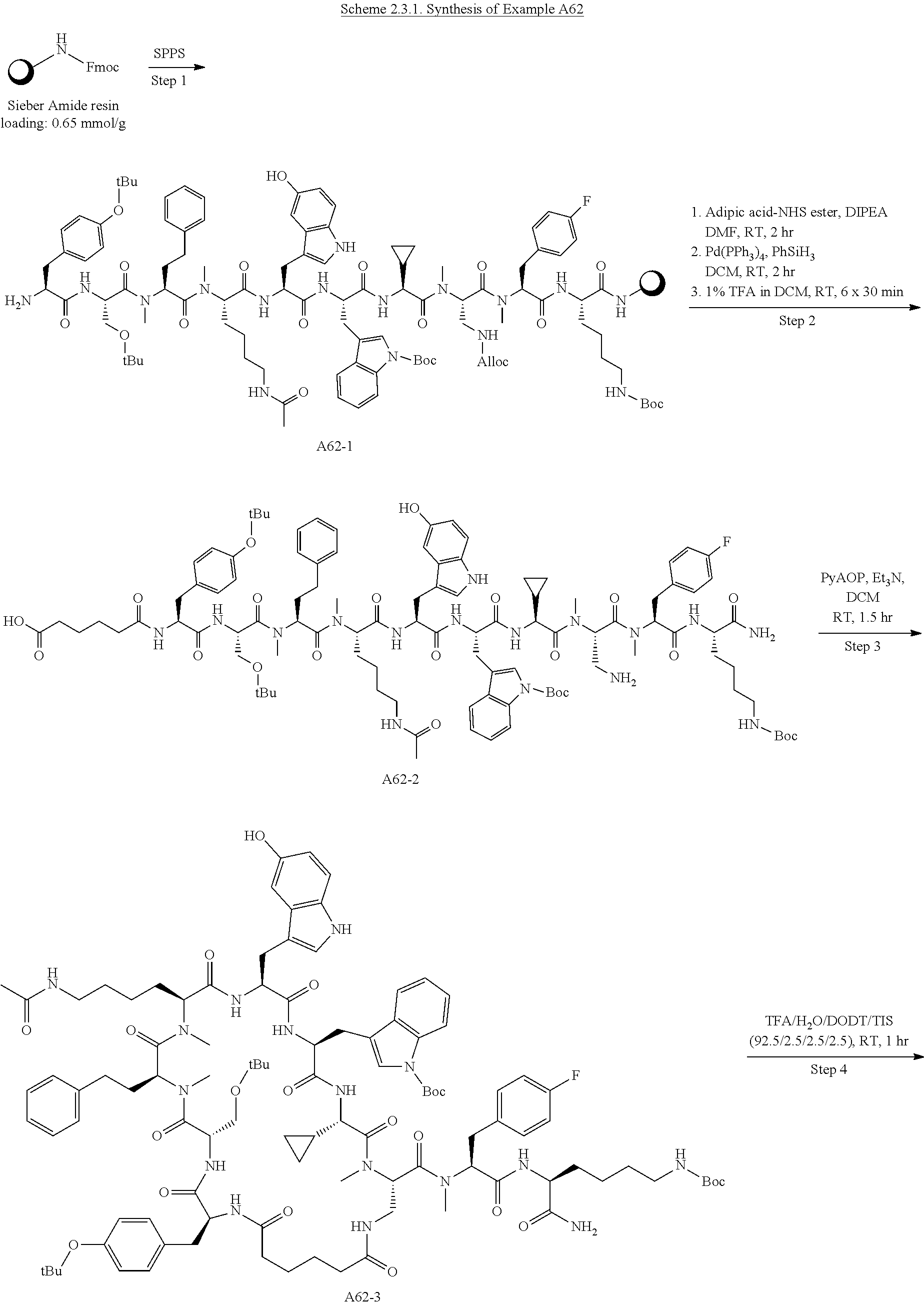

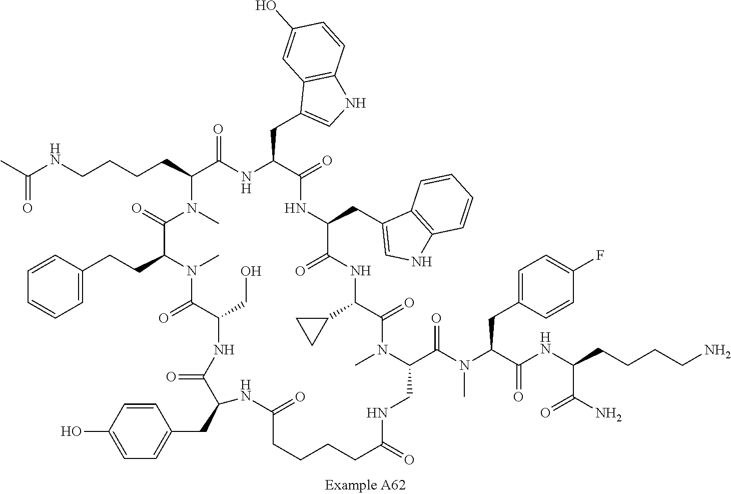 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCN)C(N)=O)CNC(=O)CCCCC(=O)N[C@@H](Cc2ccc(O)cc2)C(=O)N[C@@H](CO)C(=O)N(C)[C@@H](CCc2ccccc2)C(=O)N1C]

### Step 1. A62-1

The assembly of the linear peptide was done on the CEM Liberty Blue Synthesizer using commercial Sieber Amide Resin R3 (loading 0.65 mmol/g, 385 mg, 0.250 mmol). The couplings were carried out using the following solutions: Fmoc-amino acid (0.2 M solution DMF, 5 mL, 4 eq.), DIC (1 M solution in DMF, 2 mL, 8 eq.), Oxyma Pure® (1 M solution in DMF+0.1 M DIPEA, 1 mL, 4 eq.), addition by synthesizer. Fmoc removal was performed using a solution of pyrrolidine (5% in DMF, 10 mL), addition by synthesizer.

The amino acids were coupled with a set of different methods.
Method A Fmoc removal: 1×1 min at 90° C./Coupling: 1×4 min at 90° C.
Method B Fmoc removal: 2×10 min at RT/Coupling: 2×4 min at 90° C.
Method C Fmoc removal: 1×1 min at 90° C./Coupling: 1×2 min at 90° C.
Method D Fmoc removal: 1×10 min at RT/Coupling: 1×6 min at 50° C.

Fmoc deprotection (after Synthesis cycle 10) was performed using 5% pyrrolidine in DMF (10 mL), addition by synthesizer, 1×1 min at 90° C.

After the assembly of the linear peptide the resin was washed with DMF (5×) and DCM (5×). The amino acids and coupling methods are summarized in Table 2.22.

TABLE 2.22

| Synthesis | Amino acid | | |
|---|---|---|---|
| cycle | residue^{a} | Amino acid | Method |
| 1 | A¹⁰* | Fmoc-L-Lys(Boc)—OH | D |
| 2 | A¹⁰ | Fmoc-L-N—Me-Phe(4F)—OH | D |
| 3 | A⁹ | Fmoc-L-N—Me-Dap(Alloc)-OH | B |
| 4 | A⁸ | Fmoc-L-CyclopropylGly-OH | B |
| 5 | A⁷ | Fmoc-L-Trp(Boc)—OH | B |
| 6 | A⁶ | Fmoc-L-Trp(5-OH)—OH | A |
| 7 | A⁵ | Fmoc-L-N—Me-Lys(Ac)—OH | A |
| 8 | A⁴ | Fmoc-L-N—Me-Homo-Phe-OH | C |
| 9 | A³ | Fmoc-L-Ser(tBu)—OH | C |
| 10 | A² | Fmoc-L-Tyr(tBu)—OH | C + Fmoc |
| | | | deprotection |

^{a}Refers to the corresponding amino acid residue of cyclic peptide, P, as found, e.g., in Formulae (I), (Ia), (Ib), (Ic), (Id), or in the Table of Example III.

### Step 2. A62-2

Step 2-1: The resin from Step 1 (calculated with 250 μmol) was suspended with DMF (15 mL). Then Adipic acid-NHS ester (CAS 118380-07-7, 91.2 mg, 1.5 eq., 375 μmol), followed by DIPEA (162 mg, 218 μL, 5 eq., 1250 μmol) were added. The reaction mixture was stirred at RT for 5.5 hr. The resin was drained and washed with DMF (3×) and DCM (3×).

Step 2-2: The resin from Step 2-1 (calculated with 250 μmol) was suspended with DCM (10 mL) and treated with phenylsilane (216 mg, 248 μL, 20 eq., 2 mmol). The suspension was shaken at RT for 2 min while purged with argon. Then Pd(PPh₃)₄ (28.9 mg, 0.1 eq., 25.0 μmol) was added at RT. The reaction was agitated with argon for 3 hr. The resin was drained and washed with DMF (4×) and DCM (4×).

Step 2-3: To the resin from Step 2-2 was added 1% TFA in DCM (10 mL). The resin was shaken at RT for 30 min. The cleavage solution was filtered off and the procedure was repeated 5 times. The combined cleavage solutions were neutralized by the addition of DIPEA (slight basic pH) and concentrated to dryness in vacuo to afford the crude peptide. The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A62-2 (67.3 mg, 32 μmol, 13% yield, TFA salt) as a white solid. LCMS Method AP-1, t_{R}=5.76 min, [M+H]⁺=1961.1.

### Step 3. A62-3

A62-2 (67.3 mg, 1 eq., 32.44 μmol, TFA salt) was dissolved in DCM (60 mL) and triethylamine (32.83 mg, 45.2 μL, 10 eq., 324.4 μmol) was added. A solution of PyAOP (25.4 mg, 1.5 eq., 48.66 μmol) in DCM (2 mL) was then added dropwise. The reaction mixture was stirred at RT for 1.5 hr, then concentrated at 30° C. in vacuo to afford the crude peptide. The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A62-3 (33 mg, 17 μmol, 51% yield) as a white solid. LCMS Method AP-1, t_{R}=7.04 min, [M+H]=1943.1.

### Step 4. Example A62

A62-3 (33 mg, 1 eq., 16.99 μmol) was dissolved in TFA/H₂O/DODT/TIS (92.5/2.5/2.5/2.5) (1 mL), and the reaction mixture was stirred at RT for 1 hr. The crude peptide was precipitated with cold heptane/MTBE (1/1) (45 mL) giving a precipitate. The suspension was centrifuged, and the solvent was decanted. The product was washed with another portion of cold heptane/MTBE (1/1) (40 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A62 (4.5 mg, 2.2 μmol, 28% yield, TFA salt) as a white solid. LCMS Method AP-1, t_{R}=3.94 min, [M+2H]²⁺=815.4.

### 2.3.2 Synthesis of 4-(((3S,6S,9S,12S,15S,18S,21S,24S,27S)-9-((1H-indol-3-yl)methyl)-15-(4-acetamidobutyl)-6-cyclopropyl-3-(((S)-1-(((S)-1,6-diamino-1-oxohexan-2-yl)amino)-3-(4-fluorophenyl)-1-oxopropan-2-yl)(methyl)carbamoyl)-12-((5-hydroxy-1H-indol-3-yl)methyl)-24-(4-hydroxybenzyl)-21-(hydroxymethyl)-4,16,19-trimethyl-5,8,11,14,17,20,23,26,31-nonaoxo-18-phenethyl-1,4,7,10,13,16,19,22,25-nonaazacyclohentriacontan-27-yl)amino)-4-oxobutanoic acid TFA salt (Example A63 (A63 disclosed as SEQ ID NO: 24))

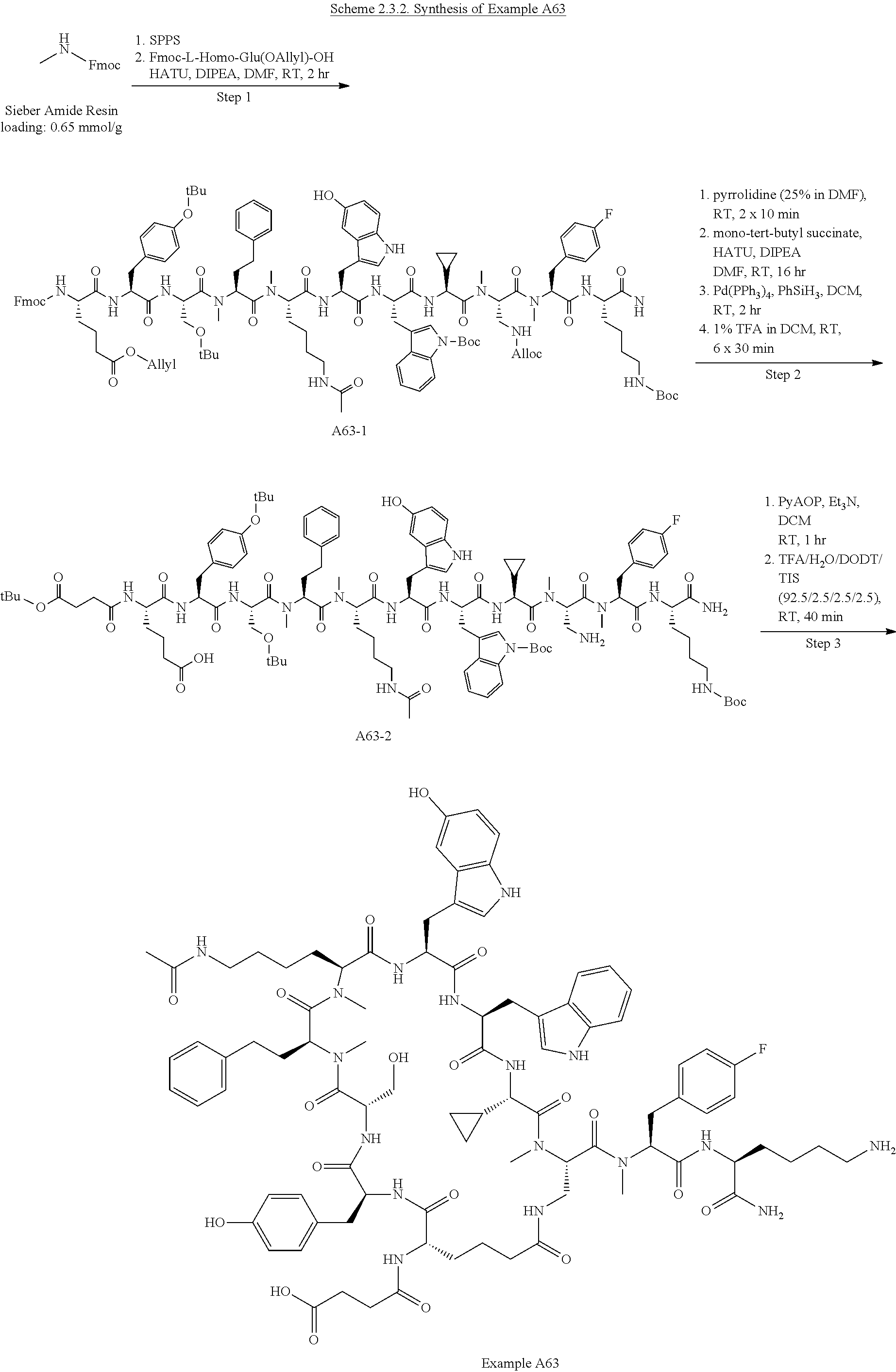

### Step 1. A63-1

The assembly of the linear peptide was done on the CEM Liberty Blue Synthesizer and by manual coupling using commercial Sieber Amide Resin R3 (loading 0.65 mmol/g, 769 mg, 0.500 mmol). The couplings were carried out using the following solutions: Fmoc-amino acid (0.2 M solution DMF, 10 mL, 4 eq.), DIC (1 M solution in DMF, 4 mL, 8 eq.), Oxyma Pure® (1 M solution in DMF+0.1 M DIPEA, 2 mL, 4 eq.), addition by synthesizer. Fmoc removal was performed using a solution of pyrrolidine (5% in DMF, 18 mL), addition by synthesizer.

The amino acids were coupled with a set of different methods.
Method A Fmoc removal: 1×2 min at 90° C./Coupling: 1×4 min at 90° C.
Method B Fmoc removal: 2×10 min at RT/Coupling: 2×4 min at 90° C.
Method C Fmoc removal: 1×10 min at RT/Coupling: 1×6 min at 50° C.

Fmoc deprotection (after Synthesis cycle 10) was performed using 5% pyrrolidine in DMF (18 mL), addition by synthesizer, 1×2 min at 90° C.

### Method D (Manual Coupling)

The resin was treated with a pre-activated solution of Fmoc-L-Homo-Glu(OAll)-OH (318 mg, 1.5 eq., 0.75 mmol), HATU (285 mg, 1.5 eq., 0.75 μmol) and DIPEA (323 mg, 435 μL, 5 eq., 2.50 mmol) in DMF (15 mL) and mixed at RT for 2 hr.

After the assembly of the linear peptide the resin was washed with DMF (5×) and DCM (5×). The amino acids and coupling methods are summarized in Table 2.23.

TABLE 2.23

| Synthesis | Amino acid | | |
|---|---|---|---|
| cycle | residue^{a} | Amino acid | Method |
| 1 | A¹⁰* | Fmoc-L-Lys(Boc)—OH | C |
| 2 | A¹⁰ | Fmoc-L-N—Me-Phe(4F)—OH | C |
| 3 | A⁹ | Fmoc-L-N—Me-Dap(Alloc)-OH | B |
| 4 | A⁸ | Fmoc-L-CyclopropylGly-OH | B |
| 5 | A⁷ | Fmoc-L-Trp(Boc)—OH | B |
| 6 | A⁶ | Fmoc-L-Trp(5-OH)—OH | A |
| 7 | A⁵ | Fmoc-L-N—Me-Lys(Ac)—OH | A |
| 8 | A⁴ | Fmoc-L-N—Me-Homo-Phe-OH | B |
| 9 | A³ | Fmoc-L-Ser(tBu)—OH | B |
| 10 | A² | Fmoc-L-Tyr(tBu)—OH | B + Fmoc |
| | | | deprotection |
| 11 | A¹ | Fmoc-L-Homo-Glu(OAll)-OH | D (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

### Step 2. A63-2

Step 2-1: The resin from Step 1 (half of the batch used and therefore calculated with 0.250 mmol) was suspended with DMF (8 mL) then treated with pyrrolidine (25% solution in DMF) (356 mg, 411 μL, 5 eq., 1.25 mmol) and shaken at RT for 10 min. The resin was drained, and the procedure repeated once. The resin was drained and washed with DMF (3×) and DCM (3×).

Step 2-2: The resin from Step 2-1 was suspended with DMF (8 mL). A solution of mono-tert-butyl succinate (CAS 15026-17-2, 218 mg, 1.5 eq., 1.25 mmol), HATU (485 mg, 5.1 eq., 1.27 mmol) and DIPEA (259 mg, 348 μL, 8 eq., 2.00 mmol) in DMF (8 mL) (pre-activated at RT for 5 min) was then added and the suspension was shaken at RT for 16 hr. The resin was drained and washed with DMF (3×) and DCM (3×).

Step 2-3: The resin from Step 2-2 was suspended with DCM (10 mL) and treated with phenylsilane (514 mg, 620 μL, 20 eq., 5 mmol). The suspension was shaken at RT for 15 min while purged with argon. Then Pd(PPh₃)₄ (57.8 mg, 0.2 eq., 0.050 mmol) was added at RT. The reaction was agitated with argon for 3 hr. The resin was drained and washed with DMF (4×) and DCM (4×).

Step 2-4: To the resin from Step 2-3 was added 1% TFA in DCM (10 mL). The resin was shaken at RT for 30 min. The cleavage solution was filtered off and the procedure was repeated 5 times. The combined cleavage solutions were neutralized by the addition of DIPEA (slight basic pH) and concentrated to dryness in vacuo to afford the crude peptide. The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford A63-2 (82.0 mg, 34 μmol, 14% yield, TFA salt) as a white solid. LCMS Method AP-1, t_{R}=6.15 min, [M+H]⁺=2132.2.

### Step 3. Example A63

Step 3-1: A63-2 (82.0 mg, 1 eq., 36.52 μmol, TFA salt) was dissolved in DCM (45 mL) and triethylamine (36.95 mg, 50.9 μL, 10 eq., 365.2 μmol) was added. A solution of PyAOP (28.56 mg, 1.5 eq., 54.77 μmol) in DCM (1 mL) was then added dropwise over a period of 5 min. The reaction mixture was stirred at RT for 1 hr, then concentrated at 30° C. in vacuo to afford the crude peptide. The crude peptide was dissolved in DMF (2 mL) and precipitated with cold MTBE (45 mL) giving a precipitate. The suspension was centrifuged, and the solvent was decanted. The product was washed with another portion of cold MTBE (40 mL). The suspension was centrifuged, and the solvent was decanted. The crude peptide was dried at RT under vacuo.

Step 3-2: The crude peptide from Step 3-1 was dissolved in TFA/H₂O/DODT/TIS (92.5/2.5/2.5/2.5) (2 mL), and the reaction mixture was stirred at RT for 40 min. The crude peptide was precipitated with cold heptane/MTBE (1/1) (45 mL) giving a precipitate. The suspension was centrifuged, and the solvent was decanted. The product was washed with another portion of cold heptane/MTBE (1/1) (40 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example A63 (7 mg, 2.3 μmol, 61% yield, TFA salt) as a white solid. LCMS Method AP-1, t_{R}=3.91 min, [M+H]⁺=1744.9.

The compounds in Table 2.24 were synthesized in analogy to Example A63.

TABLE 2.24
Examples synthesized in analogy to Example A63 (Scheme 2.3.2).

| Ex. No. | Structure/Example Name | LCMS |
|---|---|---|
| A64 (SEQ ID NO: 25) | 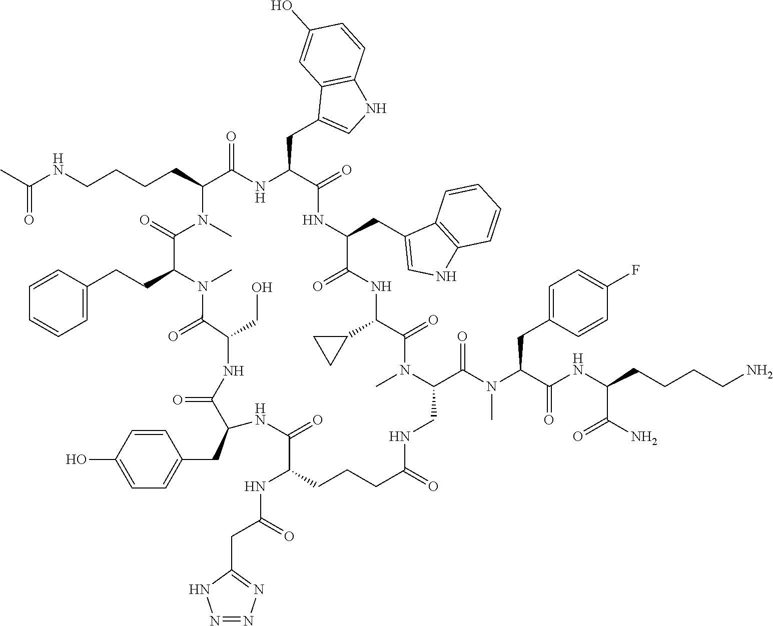 | Method AP-1 t_{R} = 3.90 min [M + H]⁺ = 1754.9 |
| | (3S,6S,9S,12S,15S,18S,21S,24S,27S)-9-((1H-indol-3-yl)methyl)-27-(2-(1H- | |
| | tetrazol-5-yl)acetamido)-15-(4-acetamidobutyl)-6-cyclopropyl-N-((S)-1-(((S)- | |
| | 1,6-diamino-1-oxohexan-2-yl)amino)-3-(4-fluorophenyl)-1-oxopropan-2-yl)- | |
| | 12-((5-hydroxy-1H-indol-3-yl)methyl)-24-(4-hydroxybenzyl)-21- | |
| | (hydroxymethyl)-N,4,16,19-tetramethyl-5,8,11,14,17,20,23,26,31-nonaoxo-18- | |
| | phenethyl-1,4,7,10,13,16,19,22,25-nonaazacyclohentriacontane-3-carboxamide | |
| | TFA salt | |

### 2.3.3 Synthesis of 2,2′,2″-(10-(2-(((S)-5-((S)-2-((3S,6 S,9S,12S,15S,18S,21S,24S,27S)-9-((1H-indol-3-yl)methyl)-27-acetamido-6-cyclopropyl-12-((5-hydroxy-1H-indol-3-yl)methyl)-15-(4-(2-hydroxyacetamido)butyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-2-oxoethyl)-1,4,7,10-tetraazacyclododecane-1,4,7-triyl)triacetic acid TFA Salt (Example A65 (A65-1 disclosed as SEQ ID NO: 114 and A65 disclosed as SEQ ID NO: 26))

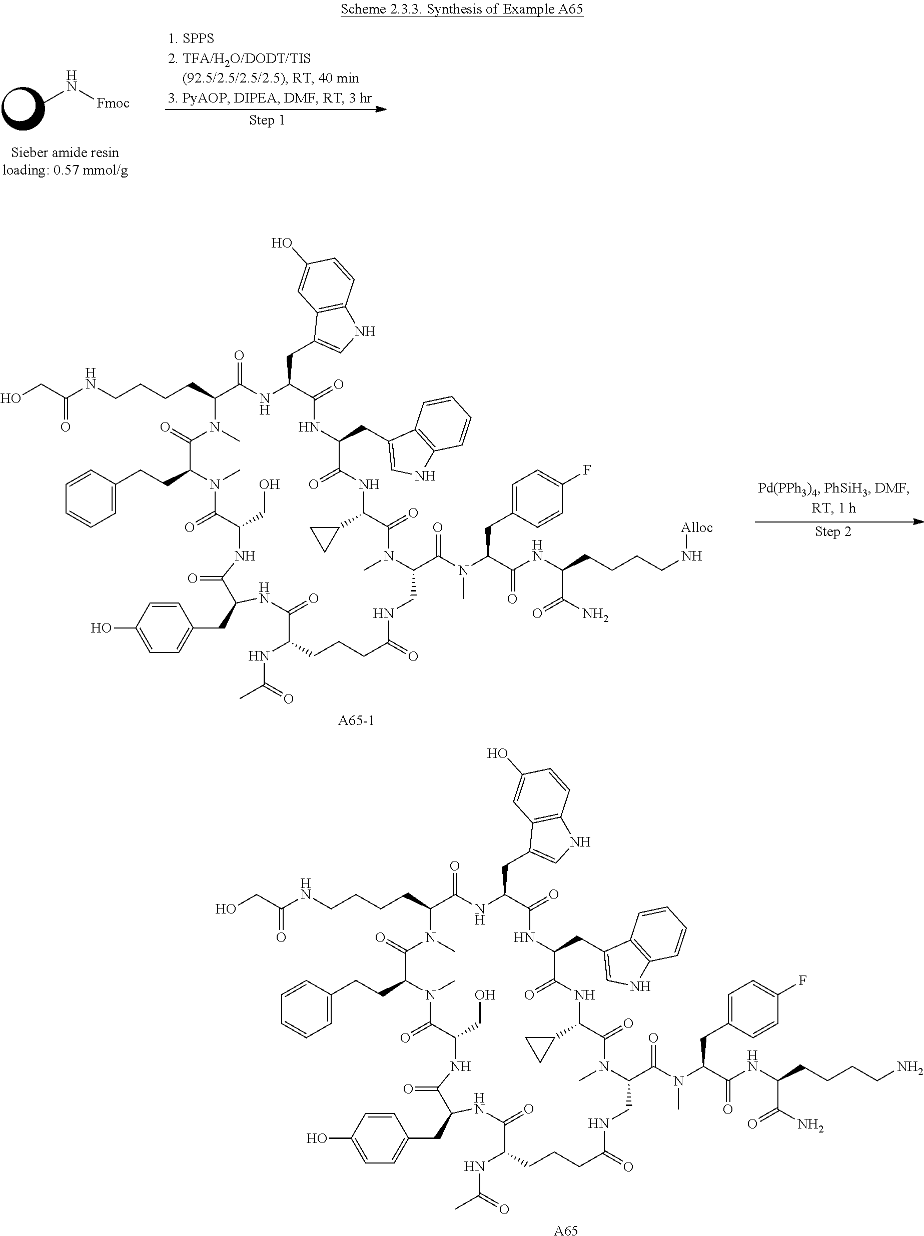

### Step 1. A65-1

Step 1-1: The assembly of the linear peptide was performed on the CEM Liberty Blue Synthesizer and by manual coupling using commercial Sieber amide resin (loading 0.57 mmol/g, 658 mg, 0.375 mmol, Watanabe Chemical). The couplings were carried out using the following solutions: Fmoc-amino acid (0.21 M solution DMF, 7.5 mL, 4.2 eq.), DIC (1 M solution in DMF, 3 mL, 8 eq.), Oxyma Pure® (0.5 M solution in DMF, 3 mL, 4 eq.), addition by synthesizer. The Fmoc removal was performed using a solution of pyrrolidine (10% in DMF, 15 mL) or 53 mM Oxyma Pure® in pyrrolidine (5.3% in DMF, 28.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 1×1 min at 90° C./Coupling: 1×30 min at 75° C.
Method F Fmoc removal: 2×1 min at RT/Coupling: 2×3 min at 90° C.

Fmoc deprotection (after Synthesis cycle 11) was performed using a solution of pyrrolidine (10% in DMF, 15 mL) or 53 mM Oxyma Pure® in pyrrolidine (5.3% in DMF, 28.5 mL), addition by synthesizer, 1×1 min at 90° C.

### Method G (Manual Coupling)

The resin was treated with a 5% solution of Ac₂O in DCM (18.75 mL) and mixed at RT for 30 min.

After the assembly of the linear peptide the resin was washed with DMF, DCM, diethyl ether, and dried in vacuo. The amino acids and coupling methods are summarized in Table 2.25.

TABLE 2.25

| Synthesis | Amino acid | | |
|---|---|---|---|
| cycle | residue^{a} | Amino acid | SPPS Method |
| 1 | A¹⁰* | Fmoc-L-Lys(Alloc)-OH | A |
| 2 | A¹⁰ | Fmoc-L-N—Me-Phe(4F)—OH | A |
| 3 | A⁹ | Fmoc-L-N—Me-Dap(Boc)—OH | B |
| 4 | A⁸ | Fmoc-L-CyclopropylGly-OH | C |
| 5 | A⁷ | Fmoc-L-Trp(Boc)—OH | D |
| 6 | A⁶ | Fmoc-L-Trp(5-OH)—OH | A |
| 7 | A⁵ | Fmoc-L-N—Me—K(COCH2O(tBu))—OH | E |
| 8 | A⁴ | Fmoc-L-N—Me-Homo-Phe-OH | B |
| 9 | A³ | Fmoc-L-Ser(Trt)—OH | F |
| 10 | A² | Fmoc-L-Tyr(tBu)—OH | D |
| 11 | A¹ | Fmoc-L-Homo-Glu(OtBu)—OH | A + Fmoc |
| | | | Deprotection |
| 12 | A¹* | Ac₂O | G (Manual |
| | | | Coupling) |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 0.375 mmol) was suspended with TFA/H₂O/DODT/TIS (92.5/2.5/2.5/2.5) (16 mL) and the suspension was shaken at RT for 40 min. The crude peptide was precipitated with hexane/diisopropyl ether (1/1) (120 mL). The suspension was centrifuged, and the solvent was decanted.

Step 1-3: The crude peptide from Step 1-2 (calculated with 0.375 mmol) was dissolved in DMF (75 mL). Then DIPEA (145 mg, 201 μL, 3 eq., 1.125 mmol) and PyAOP (CAS: 156311-83-0, 234 mg, 1.2 eq., 0.45 mmol) were added. The reaction mixture was stirred at RT for 3 hr, quenched with AcOH, and concentrated in vacuo by HT-12 (Genevac, 40° C.).

### Step 2. Example A65

The crude peptide from Step 1-3 (calculated with 0.375 mmol) was dissolved in DMF (15 mL), then phenylsilane (405 mg, 461 μL, 10 eq., 3.75 mmol) and Pd(PPh₃)₄ (43 mg, 0.1 eq., 0.038 mmol) were added. The reaction mixture was stirred at RT for 1 hr. The reaction mixture was then washed successively with DMF, DCM, diethyl ether, and dried in vacuo. The product was isolated by preparative RP HPLC (Column: XBridge C18, 5 μm, 50 mm×250 mm; Eluent A: H₂O+0.1% TFA and eluent B: ACN+0.1% TFA). Pure fractions were combined and lyophilized to afford Example A65 (70.1 mg, 0.039 mmol, 10.3% yield, TFA salt) as a white solid. LCMS Method AP-3, t_{R}=3.86 min, [M+2H]²⁺=852.4.

The compounds in Table 2.26 were synthesized in analogy to Example A65.

TABLE 2.26
Examples synthesized in analogy to Example A65 (Scheme 2.3.3).

| Ex. No. | Structure/Example Name | LCMS |
|---|---|---|
| A66 (SEQ ID NO: 27) | 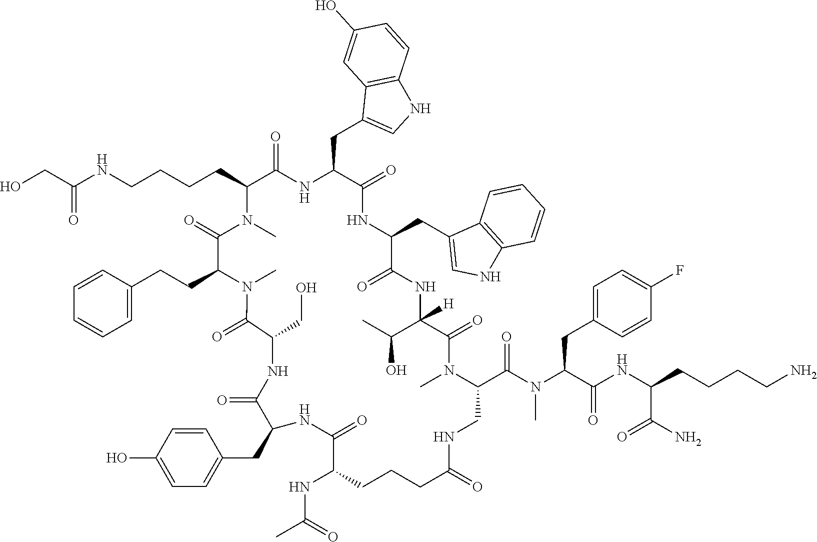 | Method AP-3 t_{R} = 3.35 min [M + 2H]²⁺ = 854.6 |
| | (3S,6S,9S,12S,15S,18S,21S,24S,27S)-9-((1H-indol-3-yl)methyl)-27-acetamido-N- | |
| | ((S)-1-(((S)-1,6-diamino-1-oxohexan-2-yl)amino)-3-(4-fluorophenyl)-1-oxopropan- | |
| | 2-yl)-12-((5-hydroxy-1H-indol-3-yl)methyl)-15-(4-(2-hydroxyacetamido)butyl)-24- | |
| | (4-hydroxybenzyl)-6-((S)-1-hydroxyethyl)-21-(hydroxymethyl)-N,4,16,19- | |
| | tetramethyl-5,8,11,14,17,20,23,26,31-nonaoxo-18-phenethyl- | |
| | 1,4,7,10,13,16,19,22,25-nonaazacyclohentriacontane-3-carboxamide TFA salt | |
| A67 (SEQ ID NO: 28) | 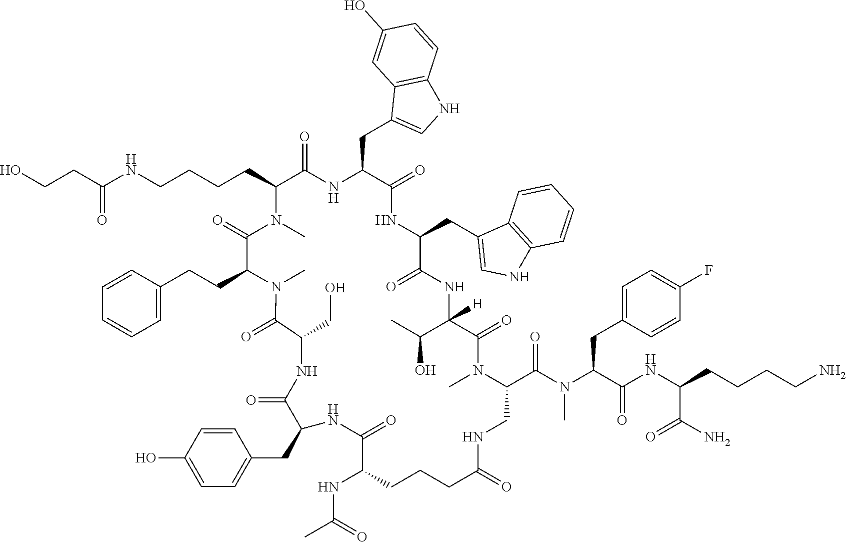 | Method AP-3 t_{R} = 3.35 min [M + 2H]²⁺ = 861.3 |
| | (3S,6S,9S,12S,15S,18S,21S,24S,27S)-9-((1H-indol-3-yl)methyl)-27-acetamido-N- | |
| | ((S)-1-(((S)-1,6-diamino-1-oxohexan-2-yl)amino)-3-(4-fluorophenyl)-1-oxopropan- | |
| | 2-y1)-12-((5-hydroxy-1H-indol-3-yl)methyl)-24-(4-hydroxybenzyl)-6-((S)-1- | |
| | hydroxyethyl)-21-(hydroxymethyl)-15-(4-(3-hydroxypropanamido)butyl)- | |
| | N,4,16,19-tetramethyl-5,8,11,14,17,20,23,26,31-nonaoxo-18-phenethyl- | |
| | 1,4,7,10,13,16,19,22,25-nonaazacyclohentriacontane-3-carboxamide TFA salt | |

### 2.3.4 Synthesis of 2-(4-(((S)-5-((S)-2-((3S,6S,9S,12S,15S,18S,21S,24S,27S)-9-((1H-indol-3-yl)methyl)-27-acetamido-15-(4-acetamidobutyl)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-6-(((R)-1,6-diamino-1-oxohexan-2-yl)amino)-6-oxohexyl)carbamoyl)piperazin-1-yl) acetic acid TFA salt (Example A68 (A68 disclosed as SEQ ID NO: 29))

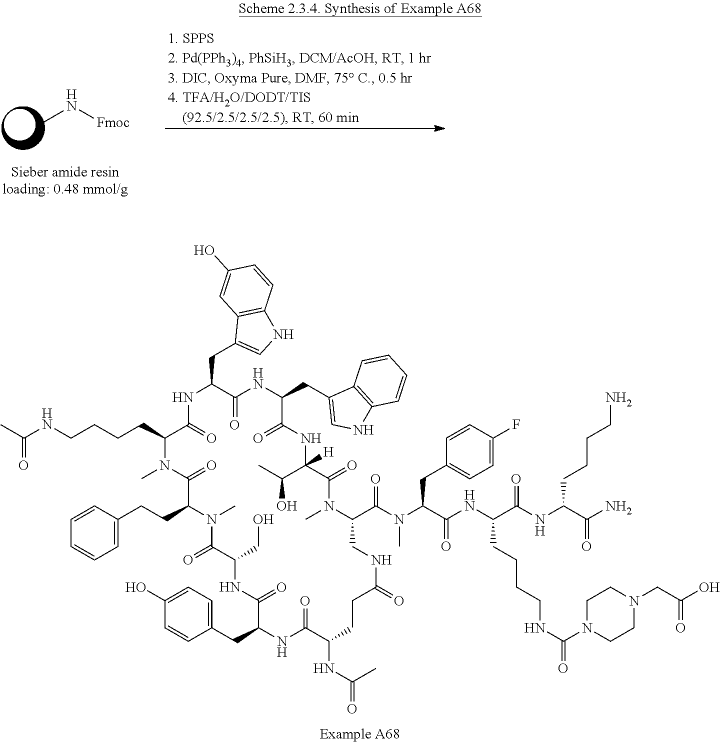

Step 1-1: The assembly of the linear peptide was performed on the CEM Liberty Blue Synthesizer and by manual coupling using commercial Sieber amide resin (loading 0.48 mmol/g, 781 mg, 0.375 mmol, Watanabe Chemical). The couplings were carried out using the following solutions: Fmoc-amino acid (0.21 M solution DMF, 7.5 mL, 4.2 eq.), DIC (1 M solution in DMF, 3 mL, 8 eq.), Oxyma Pure® (0.5 M solution in DMF, 3 mL, 4 eq.), addition by synthesizer. The Fmoc removal was performed using a solution of pyrrolidine (10% in DMF, 15 mL) or 53 mM Oxyma Pure® in pyrrolidine (5.3% in DMF, 28.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×10 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 1×10 min at 90° C.

Fmoc deprotection (after Synthesis cycle 12) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

### Method F (Manual Coupling)

The resin was treated with a 5% solution of Ac₂O in DCM (15 mL) and mixed at RT for 10 min.

After the assembly of the linear peptide the resin was washed with DMF, DCM, diethyl ether, and dried in vacuo. The amino acids and coupling methods are summarized in Table 2.27.

TABLE 2.27

| Synthesis | Amino acid | | |
|---|---|---|---|
| cycle | residue^{a} | Amino acid | SPPS Method |
| 1 | A¹⁰** | Fmoc-D-Lys(Boc)—OH | A |
| 2 | A¹⁰* | Fmoc-L-K(COpipzaa)(OtBu)—OH | A |
| 3 | A¹⁰ | Fmoc-L-N—Me-Phe(4F)—OH | A |
| 4 | A⁹ | Fmoc-L-N—Me-Dap(Alloc)-OH | B |
| 5 | A⁸ | Fmoc-L-allo-Thr(tBu)—OH | C |
| 6 | A⁷ | Fmoc-L-Trp(Boc)—OH | D |
| 7 | A⁶ | Fmoc-L-Trp(5-OH)—OH | A |
| 8 | A⁵ | Fmoc-L-N—Me-Lys(Ac)—OH | E |
| 9 | A⁴ | Fmoc-L-N—Me-Homo-Phe-OH | B |
| 10 | A³ | Fmoc-L-Ser(Trt)—OH | E |
| 11 | A² | Fmoc-L-Tyr(tBu)—OH | D |
| 12 | A¹ | Fmoc-L-Glu(OAll)-OH | A + Fmoc |
| | | | Deprotection |
| 13 | A¹* | Ac₂O | F (Manual Coupling) |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 0.375 mmol) was suspended with DCM/AcOH (15 mL, 99/1) and treated with phenylsilane (810 mg, 923 μL, 20 eq., 7.5 mmol) and Pd(PPh₃)₄ (130 mg, 0.3 eq., 0.113 mmol). The suspension was shaken at RT for 1 hr. The resin was washed successively with DCM and DMF.

Step 1-3: The resin from Step 1-2 (calculated with 0.375 mmol) was suspended with DMF (15 mL) and treated with Oxyma Pure® (426 mg, 8 eq., 3.0 mmol) and DIC (189 mg, 4 eq., 1.5 mmol). The suspension was shaken at 75° C. for 0.5 hr.

Step 1-4: The resin from Step 1-3 (calculated with 0.375 mmol) was suspended with TFA/H₂O/DODT/TIS (92.5/2.5/2.5/2.5) (15 mL) and the resin was shaken at RT for 60 min. The crude peptide was precipitated with hexane/diisopropyl ether (1/1) (120 mL). The suspension was centrifuged, and the solvent was decanted. The crude peptide was washed with diethyl ether and dried in vacuo. The product was isolated by preparative RP HPLC (Column: XBridge C18, 5 μm, 50 mm×150 mm; Eluent A: H₂O+0.1% TFA and eluent B: ACN+0.1% TFA). Pure fractions were combined and lyophilized to afford Example A68 (32.5 mg, 0.016 mmol, 4.2% yield, TFA salt) as a white solid. LCMS Method AP-3, t_{R}=3.55 min, [M+2H]²⁺=988.5.

The compounds in Table 2.28 were synthesized in analogy to Example A68.

TABLE 2.28
Examples synthesized in analogy to Example A68 (Scheme 2.3.4).

| Ex. No. | Structure/Example Name | LCMS |
|---|---|---|
| A69 (SEQ ID NO: 30) | 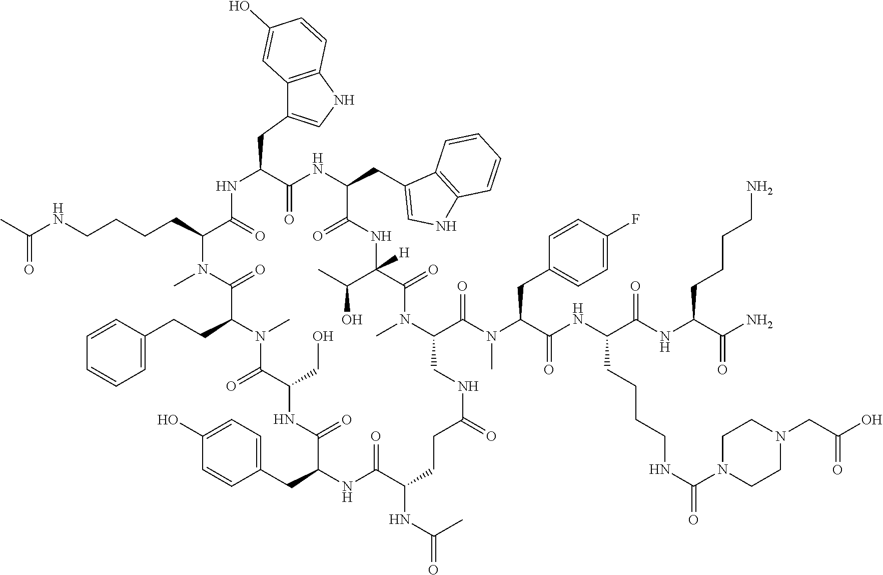 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H]([C@H](C)O)C(=O)N(C)[C@H](C(=O)N(C)[C@@H](Cc2ccc(F)cc2)C(=O)N[C@@H](CCCCNC(=O)N2CCN(CC(=O)O)CC2)C(=O)N[C@@H](CCCCN)C(N)=O)CNC(=O)CC[C@H](NC(C)=O)C(=O)N[C@@H](Cc2ccc(O)cc2)C(=O)N[C@@H](CO)C(=O)N(C)[C@@H](CCc2ccccc2)C(=O)N1C] | Method AP-3 t_{R} = 3.50 min [M + 2H]²⁺ = 988.5 |
| | 2-(4-(((S)-5-((S)-2-((3S,6S,9S,12S,15S,18S,21S,24S,27S)-9-((1H-indol-3- | |
| | y1)methyl)-27-acetamido-15-(4-acetamidobutyl)-12-((5-hydroxy-1H-indol-3- | |
| | yl)methyl)-24-(4-hydroxybenzyl)-6-((S)-1-hydroxyethyl)-21-(hydroxymethyl)- | |
| | N,4,16,19-tetramethyl-5,8,11,14,17,20,23,26,30-nonaoxo-18-phenethyl- | |
| | 1,4,7,10,13,16,19,22,25-nonaazacyclotriacontane-3-carboxamido)-3-(4- | |
| | fluorophenyl)propanamido)-6-(((S)-1,6-diamino-1-oxohexan-2-yl)amino)-6- | |
| | oxohexyl)carbamoyl)piperazin-1-yl)acetic acid TFA salt | |

### 2.3.5 Synthesis of (3S,6S,9S,12S,15S,18S,21S,24S,27S)-9-((1H-indol-3-yl)methyl)-27-acetamido-15-(4-acetamidobutyl)-N—((S)-1-(((S)-1,6-diamino-1-oxohexan-2-yl)amino)-3-(4-fluorophenyl)-1-oxopropan-2-yl)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamide TFA salt (Example A70 (A70 disclosed as SEQ ID NO: 31))

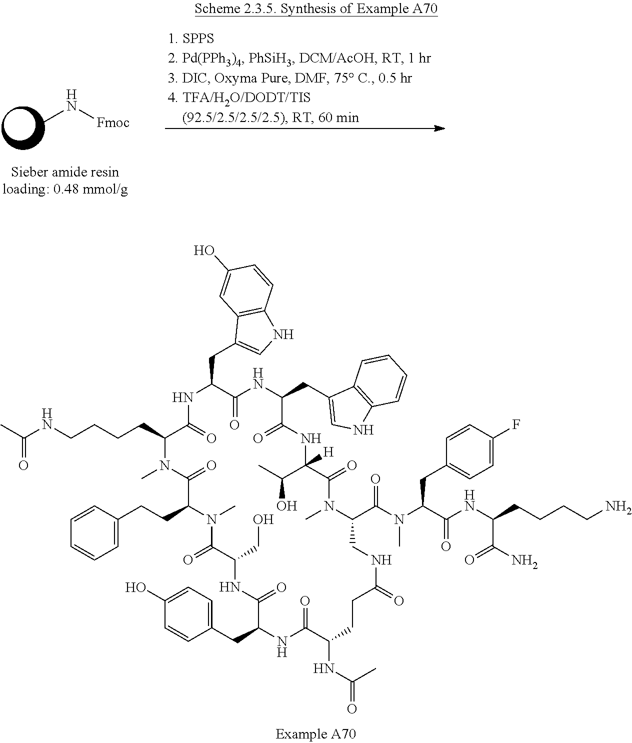

Step 1-1: The assembly of the linear peptide was performed on the CEM Liberty Blue Synthesizer and by manual coupling using commercial Sieber amide resin (loading 0.48 mmol/g, 781 mg, 0.375 mmol, Watanabe Chemical). The couplings were carried out using the following solutions: Fmoc-amino acid (0.21 M solution DMF, 7.5 mL, 4.2 eq.), DIC (1 M solution in DMF, 3 mL, 8 eq.), Oxyma Pure® (0.5 M solution in DMF, 3 mL, 4 eq.), addition by synthesizer. The Fmoc removal was performed using a solution of pyrrolidine (10% in DMF, 15 mL) or 53 mM Oxyma Pure® in pyrrolidine (5.3% in DMF, 28.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×10 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 1×10 min at 90° C.

Final Fmoc deprotection (after Synthesis cycle 11) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

### Method F (Manual Coupling)

The resin was treated with a 5% solution of Ac₂O in DCM (15 mL) and mixed at RT for 10 min.

After the assembly of the linear peptide the resin was washed with DMF, DCM, diethyl ether, and dried in vacuo. The amino acids and coupling methods are summarized in Table 2.29.

TABLE 2.29

| Synthesis | Amino acid | | SPPS |
|---|---|---|---|
| cycle | residue^{a} | Amino acid | Method |
| 1 | A¹⁰* | Fmoc-L-Lys(Boc)—OH | A |
| 2 | A¹⁰ | Fmoc-L-N—Me-Phe(4F)—OH | A |
| 3 | A⁹ | Fmoc-L-N—Me-Dap(Alloc)-OH | B |
| 4 | A⁸ | Fmoc-L-allo-Thr(tBu)—OH | C |
| 5 | A⁷ | Fmoc-L-Trp(Boc)—OH | D |
| 6 | A⁶ | Fmoc-L-Trp(5-OH)—OH | A |
| 7 | A⁵ | Fmoc-L-N—Me-Lys(Ac)—OH | E |
| 8 | A⁴ | Fmoc-L-N—Me-Homo-Phe-OH | B |
| 9 | A³ | Fmoc-L-Ser(Trt)—OH | E |
| 10 | A² | Fmoc-L-Tyr(tBu)—OH | D |
| 11 | A¹ | Fmoc-L-Glu(OAll)-OH | A + |
| | | | Final |
| 12 | A¹* | Ac₂O | F |
| | | | (Manual |
| | | | Coupling) |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 0.375 mmol) was suspended with DCM/AcOH (15 mL, 99/1), and treated with phenylsilane (810 mg, 923 μL, 20 eq., 7.5 mmol) and Pd(PPh₃)₄ (130 mg, 0.3 eq., 0.113 mmol). The suspension was shaken at RT for 1 hr. Then, the resin was washed successively with DCM and DMF.

Step 1-3: The resin from Step 1-2 (calculated with 0.375 mmol) was suspended with DMF (15 mL) and treated with Oxyma Pure® (426 mg, 8 eq., 3.0 mmol) and DIC (189 mg, 4 eq., 1.5 mmol). The suspension was shaken at 75° C. for 0.5 hr.

Step 1-4: The resin from Step 1-3 (calculated with 0.375 mmol) was suspended with TFA/H₂O/DODT/TIS (92.5/2.5/2.5/2.5) (15 mL) and the resin was shaken at RT for 60 min. The crude peptide was precipitated with hexane/diisopropyl ether (1/1) (120 mL). The suspension was centrifuged, and the solvent was decanted. The crude peptide was washed with diethyl ether and dried in vacuo. The product was isolated by preparative RP HPLC (Column: XBridge C18, 5 μm, 50 mm×150 mm; Eluent A: H₂O+0.1% TFA and eluent B: ACN+0.1% TFA). Pure fractions were combined and lyophilized to afford Example A70 (38.2 mg, 0.021 mmol, 5.7% yield, TFA salt) as a white solid. LCMS Method AP-3, t_{R}=3.98 min, [M+2H]²⁺=839.39.

The compounds in Table 2.30 were synthesized in analogy to Example A70.

TABLE 2.30
30. Examples synthesized in analogy to Example A70 (Scheme 2.3.5).

| Ex. No. | Structure/Example Name | LCMS |
|---|---|---|
| A71 | 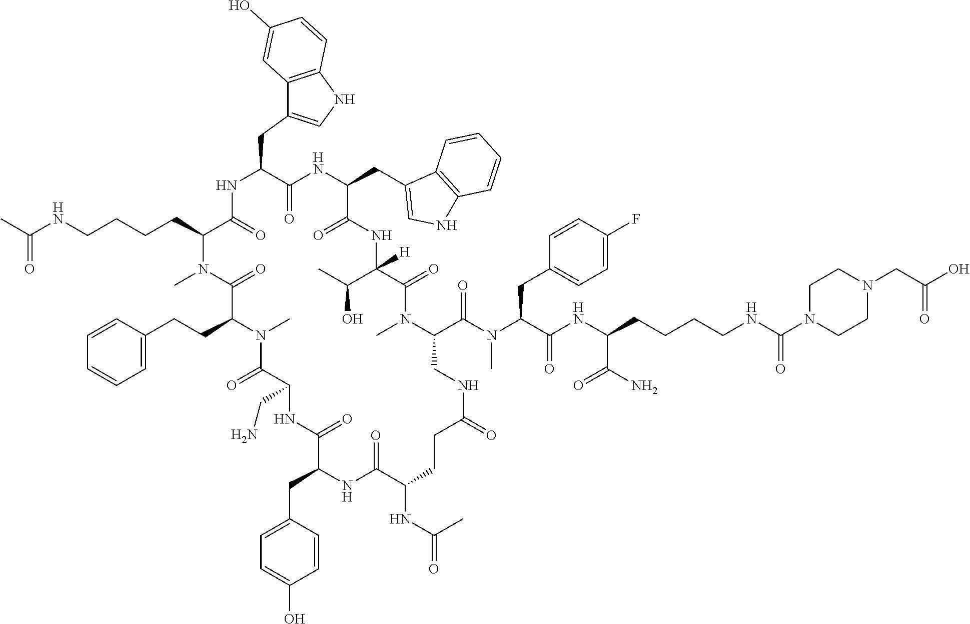 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H]([C@H](C)O)C(=O)N(C)[C@H](C(=O)N(C)[C@@H](Cc2ccc(F)cc2)C(=O)N[C@@H](CCCCNC(=O)N2CCN(CC(=O)O)CC2)C(N)=O)CNC(=O)CC[C@H](NC(C)=O)C(=O)N[C@@H](Cc2ccc(O)cc2)C(=O)N[C@@H](CN)C(=O)N(C)[C@@H](CCc2ccccc2)C(=O)N1C] | Method AP-3 t_{R} = 3.56 min [M + 2H]²⁺ = 924.0 |
| | 2-(4-(((S)-5-((S)-2-((3S,6S,9S,12S,15S,18S,21S,24S,27S)-9-((1H-indol-3- | |
| | yl)methyl)-27-acetamido-15-(4-acetamidobutyl)-21-(aminomethyl)-12-((5- | |
| | hydroxy-1H-indol-3-yl)methyl)-24-(4-hydroxybenzyl)-6-((S)-1-hydroxyethyl)- | |
| | N,4,16,19-tetramethyl-5,8,11,14,17,20,23,26,30-nonaoxo-18-phenethyl- | |
| | 1,4,7,10,13,16,19,22,25-nonaazacyclotriacontane-3-carboxamido)-3-(4- | |
| | fluorophenyl)propanamido)-6-amino-6-oxohexyl)carbamoyl)piperazin-1- | |
| | y1)acetic acid TFA salt | |

### 2.3.6 Synthesis of 2-(4-(((S)-5-((S)-2-((3S,6S,9S,12S,15S,18S,21S,24S,27S)-9-((1H-indol-3-yl)methyl)-15-(4-acetamidobutyl)-27-amino-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-6-amino-6-oxohexyl)carbamoyl)piperazin-1-yl) acetic acid TFA salt (Example A72 (A72 disclosed as SEQ ID NO: 32))

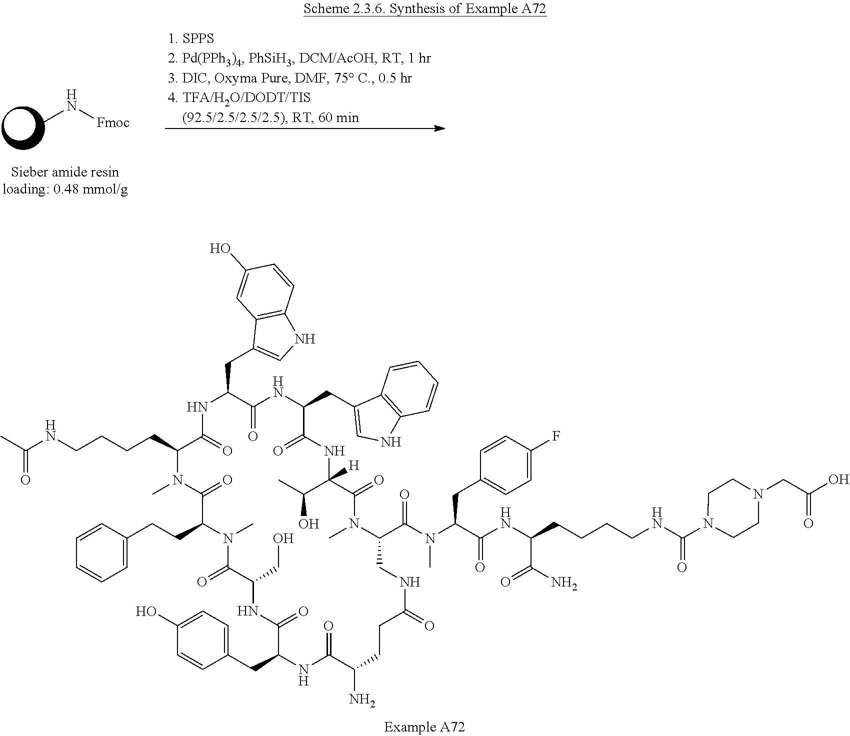

Step 1-1: The assembly of the linear peptide was performed on the CEM Liberty Blue Synthesizer using commercial Sieber amide resin (loading 0.48 mmol/g, 781 mg, 0.375 mmol, Watanabe Chemical). The couplings were carried out using the following solutions: Fmoc-amino acid (0.21 M solution DMF, 7.5 mL, 4.2 eq.), DIC (1 M solution in DMF, 3 mL, 8 eq.), Oxyma Pure® (0.5 M solution in DMF, 3 mL, 4 eq.), addition by synthesizer. The Fmoc removal was performed using a solution of pyrrolidine (10% in DMF, 15 mL) or 53 mM Oxyma Pure® in pyrrolidine (5.3% in DMF, 28.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×10 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 1×10 min at 90° C.

Fmoc deprotection (after Synthesis cycle 11) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

After the assembly of the linear peptide the resin was treated with Boc₂O (CAS: 24424-99-5, 409 mg, 5 eq., 1.875 mmol) and DIPEA (483 mg, 652 μL, 10 eq., 3.75 mmol) in DCM (15 mL) and mixed at RT for 60 min. The resin was then washed with DMF, DCM, diethyl ether, and dried in vacuo. The amino acids and coupling methods are summarized in Table 2.31.

TABLE 2.31

| Synthesis | Amino acid | | |
|---|---|---|---|
| cycle | residue^{a} | Amino acid | SPPS Method |
| 1 | A¹⁰* | Fmoc-L-K(COpipzaa)(OtBu)—OH | A |
| 2 | A¹⁰ | Fmoc-L-N—Me-Phe(4F)—OH | A |
| 3 | A⁹ | Fmoc-L-N—Me-Dap(Alloc)-OH | B |
| 4 | A⁸ | Fmoc-L-allo-Thr(tBu)—OH | C |
| 5 | A⁷ | Fmoc-L-Trp(Boc)—OH | D |
| 6 | A⁶ | Fmoc-L-Trp(5-OH)—OH | A |
| 7 | A⁵ | Fmoc-L-N—Me-Lys(Ac)—OH | E |
| 8 | A⁴ | Fmoc-L-N—Me-Homo-Phe-OH | B |
| 9 | A³ | Fmoc-L-Ser(Trt)—OH | E |
| 10 | A² | Fmoc-L-Tyr(tBu)—OH | D |
| 11 | A¹ | Fmoc-L-Glu(OAll)-OH | A + Fmoc |
| | | | deprotection |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 0.375 mmol) was suspended with DCM/AcOH (15 mL, 99/1) and treated with phenylsilane (810 mg, 923 μL, 20 eq., 7.5 mmol) and Pd(PPh₃)₄ (130 mg, 0.3 eq., 0.113 mmol). The suspension was shaken at RT for 1 hr. Then, the resin was washed successively with DCM and DMF.

Step 1-3: The resin from Step 1-2 (calculated with 0.375 mmol) was suspended with DMF (15 mL) and treated with Oxyma Pure® (426 mg, 8 eq., 3.0 mmol) and DIC (189 mg, 4 eq., 1.5 mmol) in DMF (15 mL). The suspension was shaken at 75° C. for 0.5 hr.

Step 1-4: The resin from Step 1-3 (calculated with 0.375 mmol) was suspended with TFA/H₂O/DODT/TIS (92.5/2.5/2.5/2.5) (15 mL) and the resin was shaken at RT for 60 min. The crude peptide was precipitated with hexane/diisopropyl ether (1/1) (120 mL). The suspension was centrifuged, and the solvent was decanted. The crude peptide was washed with diethyl ether and dried in vacuo. The product was isolated by preparative RP HPLC (Column: XBridge C18, 5 μm, 50 mm×150 mm; Eluent A: H₂O+0.1% TFA and eluent B: ACN+0.1% TFA). Pure fractions were combined and lyophilized to afford Example A72 (27.5 mg, 0.014 mmol, 3.8% yield, TFA salt) as a white solid. LCMS Method AP-3, t_{R}=3.56 min, [M+2H]²⁺=903.48.

### 2.3.7 Synthesis of 2-(4-(((S)-5-((S)-2-((3S,6S,9S,12S,15S,18S,21S,24S,27S)-9-((1H-indol-3-yl)methyl)-15-(4-acetamidobutyl)-27-(3-aminopropanamido)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-6-amino-6-oxohexyl)carbamoyl)piperazin-1-yl) acetic acid TFA salt (Example A73 (A73 disclosed as SEQ ID NO: 121))

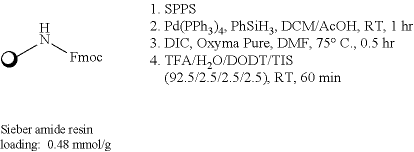

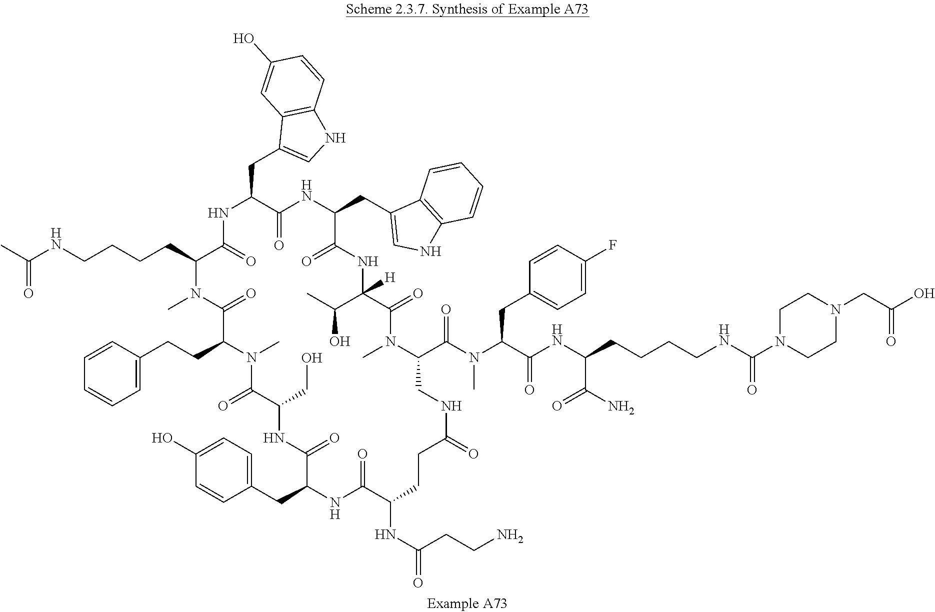 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H]([C@H](C)O)C(=O)N(C)[C@H](C(=O)N(C)[C@@H](Cc2ccc(F)cc2)C(=O)N[C@@H](CCCCNC(=O)N2CCN(CC(=O)O)CC2)C(N)=O)CNC(=O)CC[C@H](NC(=O)CCN)C(=O)N[C@@H](Cc2ccc(O)cc2)C(=O)N[C@@H](CO)C(=O)N(C)[C@@H](CCc2ccccc2)C(=O)N1C]

Step 1-1: The assembly of the linear peptide was performed on the CEM Liberty Blue Synthesizer using commercial Sieber amide resin (loading 0.48 mmol/g, 781 mg, 0.375 mmol, Watanabe Chemical). The couplings were carried out using the following solutions: Fmoc-amino acid (0.21 M solution DMF, 7.5 mL, 4.2 eq.), DIC (1 M solution in DMF, 3 mL, 8 eq.), Oxyma Pure® (0.5 M solution in DMF, 3 mL, 4 eq.), addition by synthesizer. The Fmoc removal was performed using a solution of pyrrolidine (10% in DMF, 15 mL) or 53 mM Oxyma Pure® in pyrrolidine (5.3% in DMF, 28.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×10 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 1×10 min at 90° C.

After the assembly of the linear peptide the resin was washed with DMF, DCM, diethyl ether, and dried in vacuo. The amino acids and coupling methods are summarized in Table 2.32.

TABLE 2.32

| Synthesis | Amino acid | | |
|---|---|---|---|
| cycle | residue^{a} | Amino acid | SPPS Method |
| 1 | A¹⁰* | Fmoc-L-K(COpipzaa)(OtBu)—OH | A |
| 2 | A¹⁰ | Fmoc-L-N—Me-Phe(4F)—OH | A |
| 3 | A⁹ | Fmoc-L-N—Me-Dap(Alloc)-OH | B |
| 4 | A⁸ | Fmoc-L-allo-Thr(tBu)—OH | C |
| 5 | A⁷ | Fmoc-L-Trp(Boc)—OH | D |
| 6 | A⁶ | Fmoc-L-Trp(5-OH)—OH | A |
| 7 | A⁵ | Fmoc-L-N—Me-Lys(Ac)—OH | E |
| 8 | A⁴ | Fmoc-L-N—Me-Homo-Phe-OH | B |
| 9 | A³ | Fmoc-L-Ser(Trt)—OH | E |
| 10 | A² | Fmoc-L-Tyr(tBu)—OH | D |
| 11 | A¹ | Fmoc-L-Glu(OAll)-OH | A |
| 12 | A¹* | Boc-L-Beta-Ala-OH | D |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 0.375 mmol) was suspended with DCM/AcOH (15 mL, 99/1) and treated with phenylsilane (810 mg, 923 μL, 20 eq., 7.5 mmol) and Pd(PPh₃)₄ (130 mg, 0.3 eq., 0.113 mmol). The suspension was shaken at RT for 1 hr. Then, the resin was washed successively with DCM and DMF.

Step 1-3: The resin from Step 1-2 (calculated with 0.375 mmol) was suspended with DMF (15 mL) and treated with Oxyma Pure® (426 mg, 8 eq., 3.0 mmol) and DIC (189 mg, 4 eq., 1.5 mmol). The suspension was shaken at 75° C. for 0.5 hr.

Step 1-4: The resin from Step 1-3 (calculated with 0.375 mmol) was suspended with TFA/H₂O/DODT/TIS (92.5/2.5/2.5/2.5) (15 mL) and the resin was shaken at RT for 60 min. The crude peptide was precipitated with hexane/diisopropyl ether (1/1) (120 mL). The suspension was centrifuged, and the solvent was decanted. The crude peptide was washed with diethyl ether and dried in vacuo. The product was isolated by preparative RP HPLC (Column: XBridge C18, 5 μm, 50 mm×150 mm; Eluent A: H₂O+0.1% TFA and eluent B: ACN+0.1% TFA). Pure fractions were combined and lyophilized to afford Example A73 (35.3 mg, 0.018 mmol, 4.7% yield, TFA salt) as a white solid. LCMS Method AP-3, t_{R}=3.61 min, [M+2H]²⁺=938.99.

### 3 Coupling of the Chelators to the Parent Peptide (“B” Examples)

### 3.1 Coupling of DOTA

### 3.1.1 Synthesis of 2,2′,2″-(10-(2-(((S)-5-((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-2-oxoethyl)-1,4,7,10-tetraazacyclododecane-1,4,7-triyl)triacetic acid TFA salt (Example B1 (A40 disclosed as SEQ ID NO: 14 and B1 disclosed as SEQ ID NO: 36))

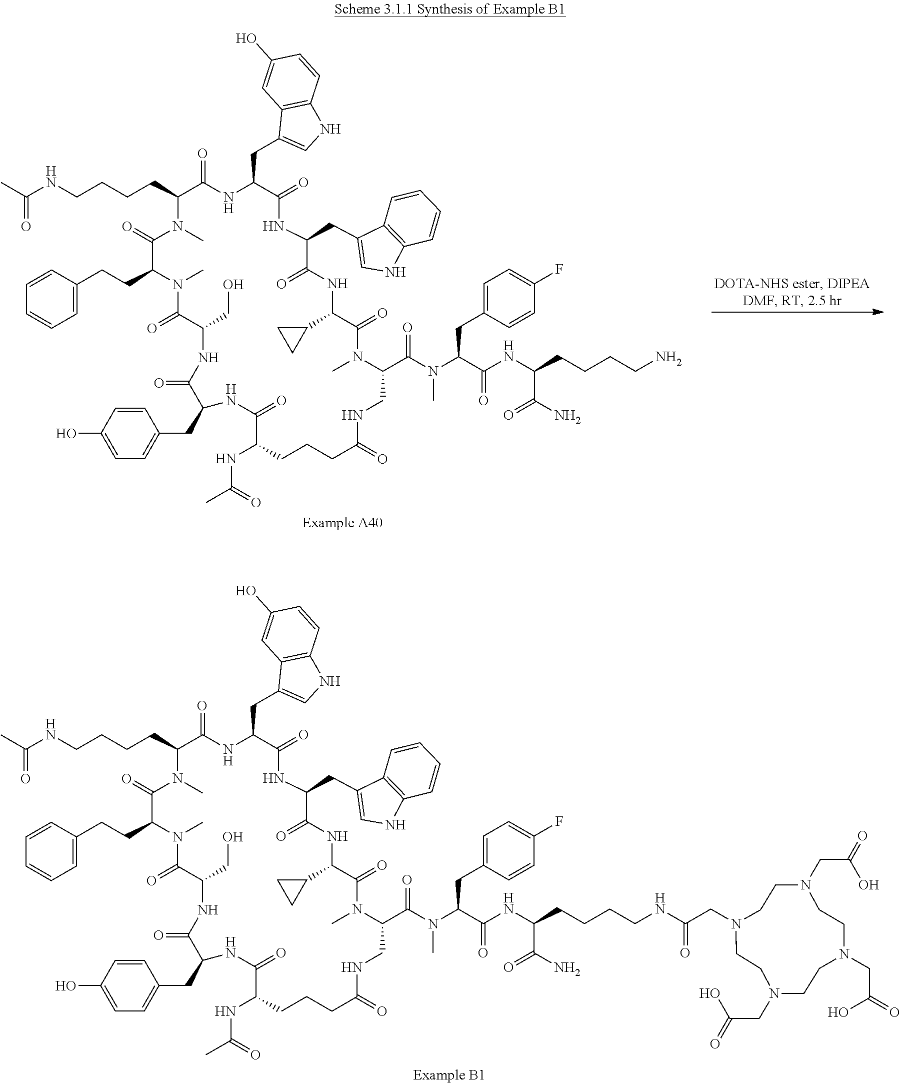

To a clear colorless solution of Example A40 (250 mg, 1 eq., 138.8 μmol, TFA salt) and DOTA-NHS ester (158.6 mg, 1.5 eq., 208.2 μmol, hexafluorophosphate-trifluoroacetate) in DMF (2.0 mL) at RT was added DIPEA (89.71 mg, 121 μL, 5 eq., 694.1 μmol). The resulting clear solution was stirred at RT for 2 hr. The reaction mixture was precipitated with cold diethyl ether (45 mL) giving a precipitate. The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example B1 (279 mg, 110 μmol, 79% yield, TFA salt) as a white solid. LCMS Method AP-1, t_{R}=3.80 min, [M+H]⁺=2074.0.

The following examples (for structures, see Table 3.1) were synthesized in analogy to Example B1:

Example B2 was synthesized from Example A43.

Example B3 was synthesized from Example A45.

Example B4 was synthesized from Example A48.

Example B9 was synthesized from Example A75.

TABLE 3.1
Examples synthesized in analogy to Example B1 (Scheme 3.1.1).

| Ex. No. | Structure/Example Name | LCMS |
|---|---|---|
| B2 (SEQ ID NO: 37) | 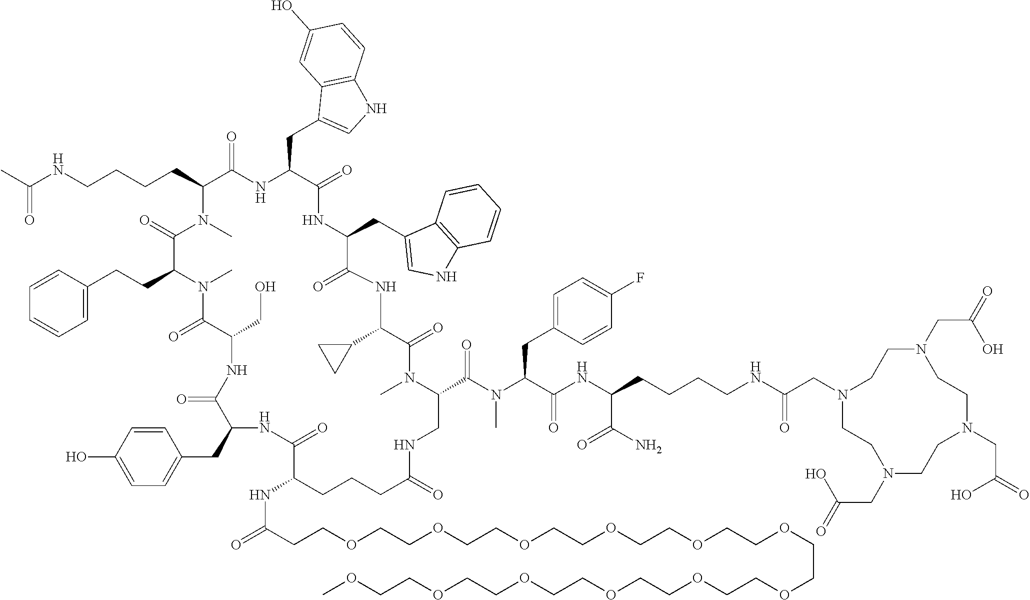 [SMILES: COCCOCCOCCOCCOCCOCCOCCOCCOCCOCCOCCOCCC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(C)=O)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.17 min [M + H]⁺ = 2602.3 |
| | 2,2′,2″-(10-(2-(((S)-5-((S)-2-((3S,6S,9S,12S,15S,18S,21S,24S,27S)-9-((1H- | |
| | indol-3-yl)methyl)-15-(4-acetamidobutyl)-6-cyclopropyl-12-((5-hydroxy-1H- | |
| | indol-3-y1)methyl)-24-(4-hydroxybenzyl)-21-(hydroxymethyl)-N,4,16,19- | |
| | tetramethyl-27-(2,5,8,11,14,17,20,23,26,29,32,35-dodecaoxaoctatriacontan-38- | |
| | amido)-5,8,11,14,17,20,23,26,31-nonaoxo-18-phenethyl- | |
| | 1,4,7,10,13,16,19,22,25-nonaazacyclohentriacontane-3-carboxamido)-3-(4- | |
| | fluorophenyl)propanamido)-6-amino-6-oxohexyl)amino)-2-oxoethyl)-1,4,7,10- | |
| | tetraazacyclododecane-1,4,7-triyl)triacetic acid TFA salt | |
| B3 (SEQ ID NO: 38) | 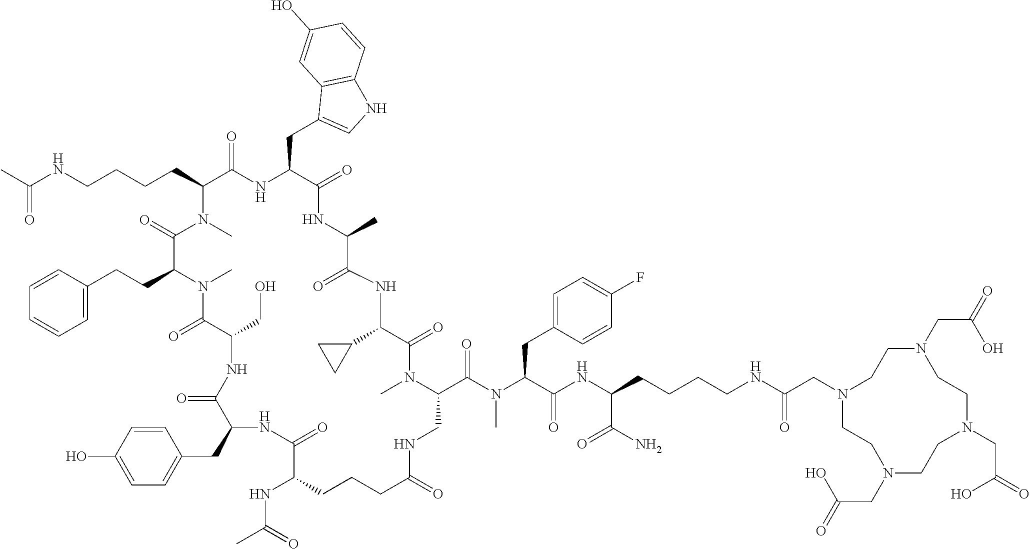 [SMILES: CC(=O)NCCCC[C@H]1C(=O)N[C@@H](Cc2c[nH]c3ccc(O)cc23)C(=O)N[C@@H](C)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 3.37 min [M + H]⁺ = 1959.1 |
| | 2,2′,2″-(10-(2-((5-((S)-2-((3S,6S,9S,12S,15S,18S,21S,24S,27S)-27- | |
| | acetamido-15-(4-acetamidobutyl)-6-cyclopropyl-12-((5-hydroxy-1H-indol-3- | |
| | yl)methyl)-24-(4-hydroxybenzyl)-21-(hydroxymethyl)-N,4,9,16,19- | |
| | pentamethyl-5,8,11,14,17,20,23,26,31-nonaoxo-18-phenethyl- | |
| | 1,4,7,10,13,16,19,22,25-nonaazacyclohentriacontane-3-carboxamido)-3-(4- | |
| | fluorophenyl)propanamido)-6-amino-6-oxohexyl)amino)-2-oxoethyl)-1,4,7,10- | |
| | tetraazacyclododecane-1,4,7-triyl)triacetic acid TFA salt | |
| B4 (SEQ ID NO: 39) | 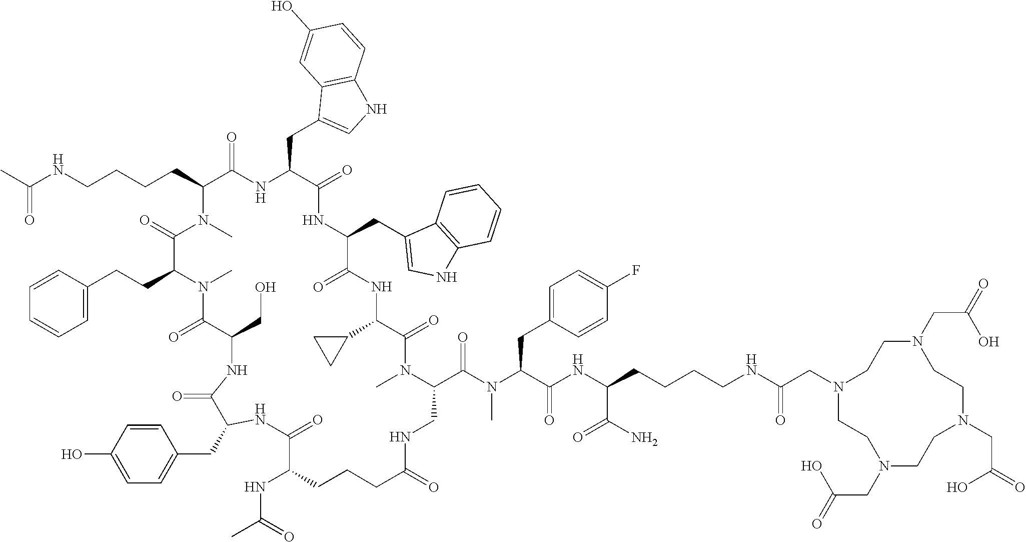 [SMILES: CC(=O)NCCCC[C@H]1C(=O)N[C@@H](Cc2c[nH]c3ccc(O)cc23)C(=O)N[C@@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@H](Cc2ccc(O)cc2)C(=O)N[C@H](CO)C(=O)N(C)[C@@H](CCc2ccccc2)C(=O)N1C] | Method AP-1 t_{R} = 3.99 min [M + H]⁺ = 2074.0 |
| | 2,2′,2″-(10-(2-(((S)-5-((S)-2-((3S,6S,9S,12S,15S,18S,21R,24R,27S)-9-((1H- | |
| | indol-3-yl)methyl)-27-acetamido-15-(4-acetamidobutyl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,31-nonaoxo-18-phenethyl- | |
| | 1,4,7,10,13,16,19,22,25-nonaazacyclohentriacontane-3-carboxamido)-3-(4- | |
| | fluorophenyl)propanamido)-6-amino-6-oxohexyl)amino)-2-oxoethyl)-1,4,7,10- | |
| | tetraazacyclododecane-1,4,7-triyl)triacetic acid TFA salt | |
| B9 (SEQ ID NO: 43) | 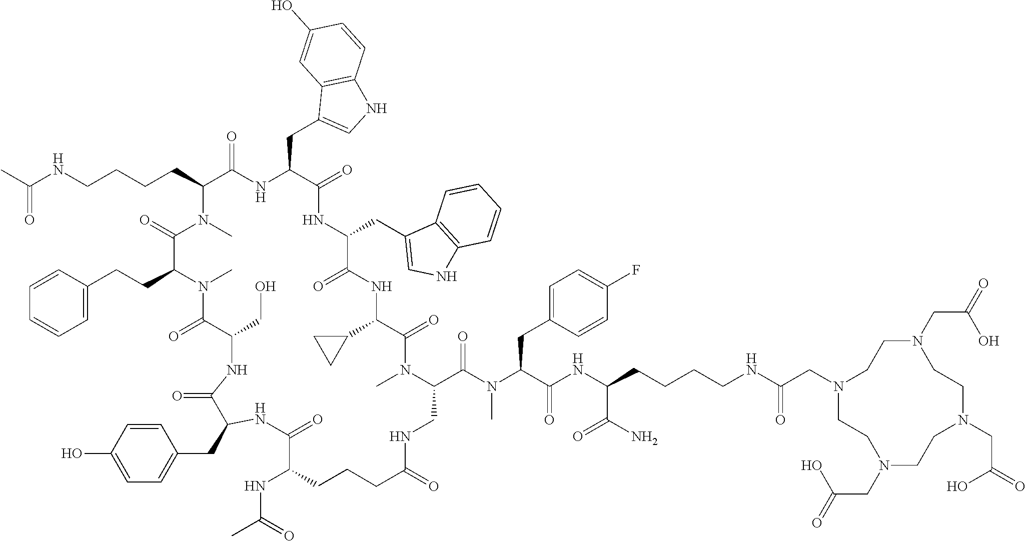 [SMILES: CC(=O)NCCCC[C@H]1C(=O)N[C@@H](Cc2c[nH]c3ccc(O)cc23)C(=O)N[C@H](Cc2c[nH]c3ccccc23)C(=O)N[C@@H](C2CC2)C(=O)N(C)[C@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)CNC(=O)CCC[C@H](NC(C)=O)C(=O)N[C@@H](Cc2ccc(O)cc2)C(=O)N[C@@H](CO)C(=O)N(C)[C@@H](CCc2ccccc2)C(=O)N1C] | Method AP-1 t_{R} = 3.81 min [M + H]⁺ = 2074.0 |
| | 2,2',2″-(10-(2-(((S)-5-((S)-2-((3S,6S,9R,12S,15S,18S,21S,24S,27S)-9-((1H- | |
| | indol-3-yl)methyl)-27-acetamido-15-(4-acetamidobutyl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-5,8,11,14,17,20,23,26,31-nonaoxo-18-phenethyl- | |
| | 1,4,7,10,13,16,19,22,25-nonaazacyclohentriacontane-3-carboxamido)-3-(4- | |
| | fluorophenyl)propanamido)-6-amino-6-oxohexyl)amino)-2-oxoethyl)-1,4,7,10- | |
| | tetraazacyclododecane-1,4,7-triyl)triacetic acid TFA salt | |

### 3.1.2 Synthesis of 2,2′,2″-(10-(2-(((S)-5-((S)-2-((3S,6S,9S,12S,15S,18S,21S,24S,27S)-9-((1H-indol-3-yl)methyl)-27-acetamido-6-cyclopropyl-12-((5-hydroxy-1H-indol-3-yl)methyl)-15-(4-(2-hydroxyacetamido)butyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-2-oxoethyl)-1,4,7,10-tetraazacyclododecane-1,4,7-triyl)triacetic acid TFA salt (Example B5 (A65 disclosed as SEQ ID NO: 26 and B5 disclosed as SEQ ID NO: 40))

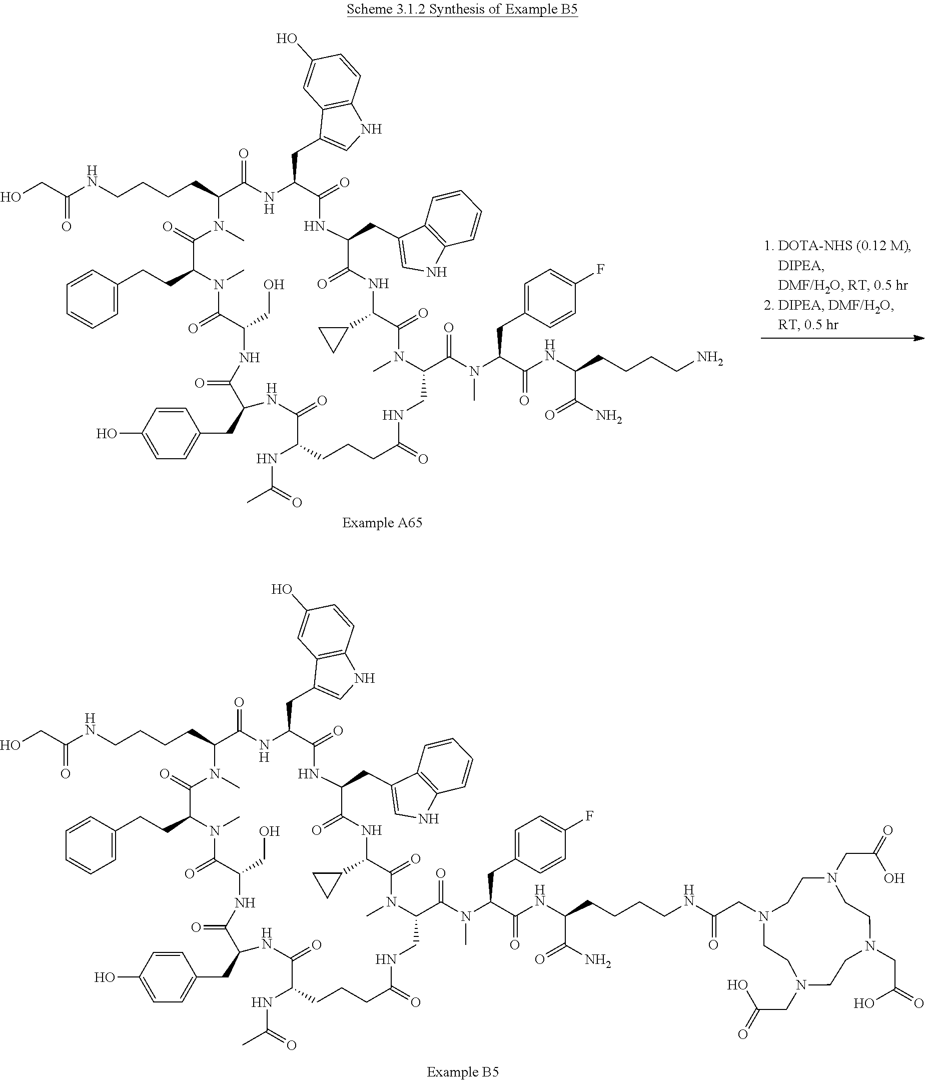

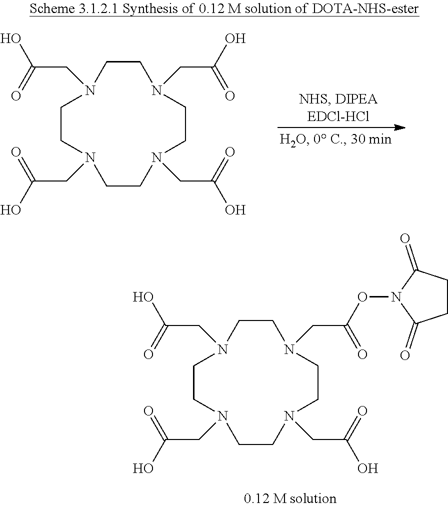 [SMILES: O=C(O)CN1CCN(CC(=O)O)CCN(CC(=O)O)CCN(CC(=O)O)CC1 ; O=C(O)CN1CCN(CC(=O)O)CCN(CC(=O)ON2C(=O)CCC2=O)CCN(CC(=O)O)CC1]

To a solution of DOTA (CAS: 60239-18-1, 0.910 g, 1.5 eq., 2.25 mmol), N-hydroxysuccinimide (CAS: 6066-82-6, 0.173 g, 1 eq., 1.5 mmol), and DIPEA (0.393 ml, 1.5 eq., 2.25 mmol) in H₂O (8.33 ml) was slowly added a solution of 0.36 M EDCI-HCl (0.288 g, 1 eq., 1.5 mmol) in H₂O (4.17 ml) at 0° C. The reaction mixture was stirred at 0° C. for 30 min, and then used for the next reaction without further purification.

Step 1-1: Example A65 (30 mg, 1 eq., 16.5 μmol, TFA salt) was dissolved in DMF (0.83 mL), then DIPEA (19.2 mg, 26 μL, 9 eq., 149 μmol) and a 0.12 M solution of DOTA-NHS-ester prepared as described above (826 μL, 6 eq., 99.1 μmol) were added. The reaction mixture was stirred at RT for 0.5 hr.

Step 1-2: DIPEA (19.2 mg, 26 μL, 9 eq., 149 μmol) was added to the reaction mixture from Step 1-1. After the reaction mixture was stirred at RT for an additional 0.5 hr, the product was isolated by preparative RP HPLC (Column: XBridge® C18, OBD™ Prep Column, 130 Å, 5 μm, 19 mm×150 mm; Eluent A: H₂O+0.1% TFA and eluent B: ACN+0.1% TFA). Pure fractions were combined and lyophilized to afford Example B5 (12.0 mg, 5.44 μmol, 33% yield, TFA salt) as a white solid. LCMS Method AP-3, t_{R}=3.80 min, [M+2H]²⁺=1045.5.

The following examples (for structures, see Table 3.2) were synthesized in analogy to Example B5:

Example B6 was synthesized from Example A66.

Example B7 was synthesized from Example A67.

TABLE 3.2
Examples synthesized in analogy to Example B5 (Scheme 3.1.2).

| Ex. No. | Structure/Example Name | LCMS |
|---|---|---|
| B6 (SEQ ID NO: 41) | 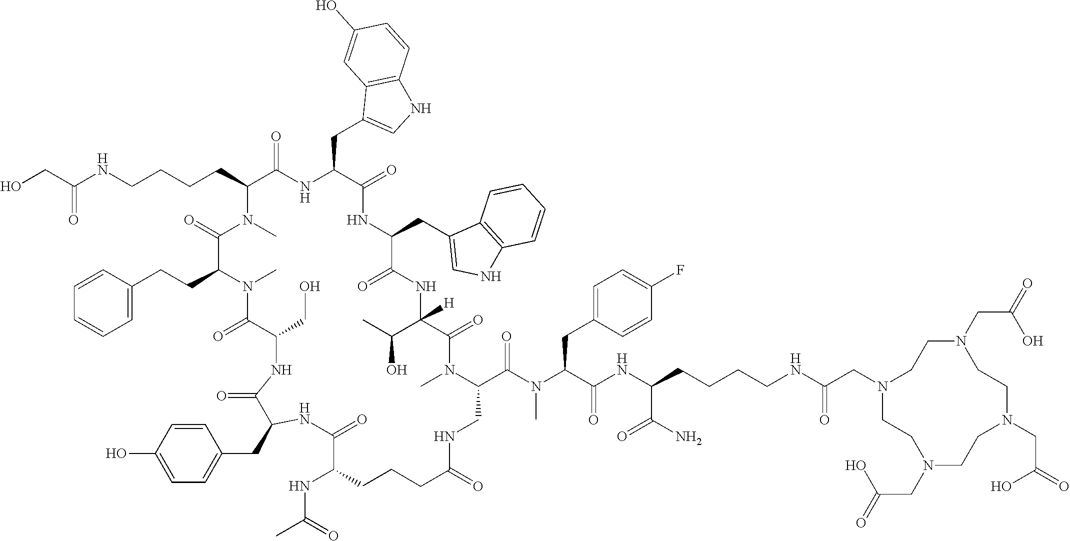 [SMILES: CC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H]([C@H](C)O)NC(=O)[C@H](Cc2c[nH]c3ccccc23)NC(=O)[C@H](Cc2c[nH]c3ccc(O)cc23)NC(=O)[C@H](CCCCNC(=O)CO)N(C)C(=O)[C@H](CCc2ccccc2)N(C)C(=O)[C@H](CO)NC(=O)[C@H](Cc2ccc(O)cc2)NC1=O] | Method AP-3 t_{R} = 3.53 min [M + 2H]²⁺ = 1047.6 |
| | 2,2′,2″-(10-(2-((((S)-2-((3S,6S,9S,12S,15S,18S,21S,24S,27S)-9-((1H-indol- | |
| | 3-yl)methyl)-27-acetamido-12-((5-hydroxy-1H-indol-3-yl)methyl)-15-(4-(2- | |
| | hydroxyacetamido)butyl)-24-(4-hydroxybenzyl)-6-((S)-1-hydroxyethyl)-21- | |
| | (hydroxymethyl)-N,4,16,19-tetramethyl-5,8,11,14,17,20,23,26,31-nonaoxo-18- | |
| | phenethyl-1,4,7,10,13,16,19,22,25-nonaazacyclohentriacontane-3- | |
| | carboxamido)-3-(4-fluorophenyl)propanamido)-6-amino-6-oxohexyl)amino)-2- | |
| | oxoethyl)-1,4,7,10-tetraazacyclododecane-1,4,7-triy1)triacetic acid TFA salt | |
| B7 (SEQ ID NO: 42) | 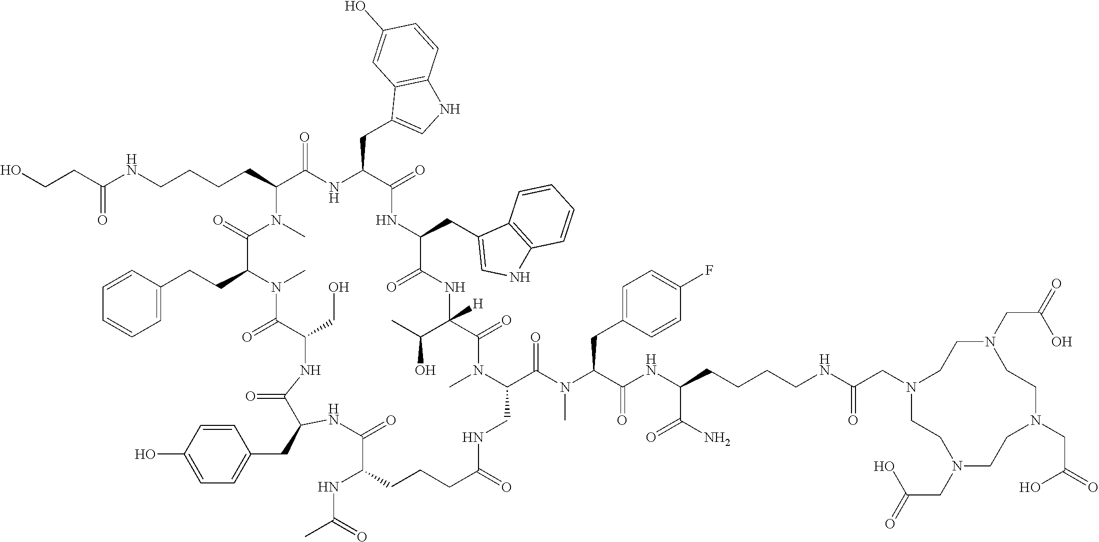 [SMILES: CC(=O)N[C@H]1CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H]([C@H](C)O)NC(=O)[C@H](Cc2c[nH]c3ccccc23)NC(=O)[C@H](Cc2c[nH]c3ccc(O)cc23)NC(=O)[C@H](CCCCNC(=O)CCO)N(C)C(=O)[C@H](CCc2ccccc2)N(C)C(=O)[C@H](CO)NC(=O)[C@H](Cc2ccc(O)cc2)NC1=O] | Method AP-3 t_{R} = 3.53 min [M + 2H]²⁺ = 1054.6 |
| | 2,2′,2″-(10-(2-(((S)-5-((S)-2-((3S,6S,9S,12S,15S,18S,21S,24S,27S)-9-((1H-indol- | |
| | 3-yl)methyl)-27-acetamido-12-((5-hydroxy-1H-indol-3-yl)methyl)-24-(4- | |
| | hydroxybenzyl)-6-((S)-1-hydroxyethyl)-21-(hydroxymethyl)-15-(4-(3- | |
| | hydroxypropanamido)butyl)-N,4,16,19-tetramethyl-5,8,11,14,17,20,23,26,31- | |
| | nonaoxo-18-phenethyl-1,4,7,10,13,16,19,22,25-nonaazacyclohentriacontane-3- | |
| | carboxamido)-3-(4-fluorophenyl)propanamido)-6-amino-6-oxohexyl)amino)-2- | |
| | oxoethyl)-1,4,7,10-tetraazacyclododecane-1,4,7-triyl)triacetic acid TFA salt | |

### 3.1.3 Synthesis of Example B8

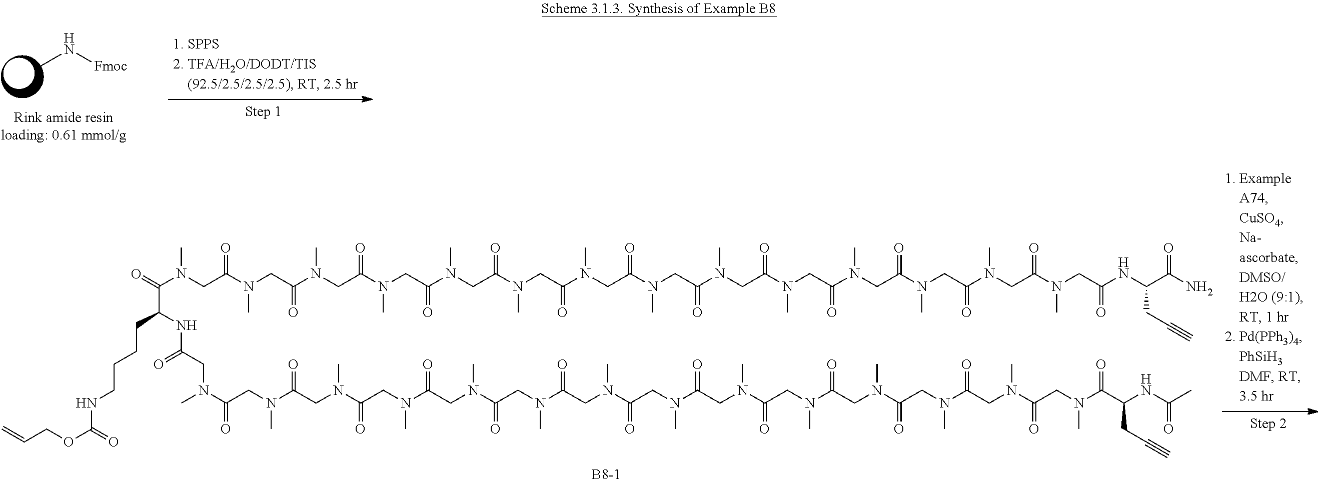

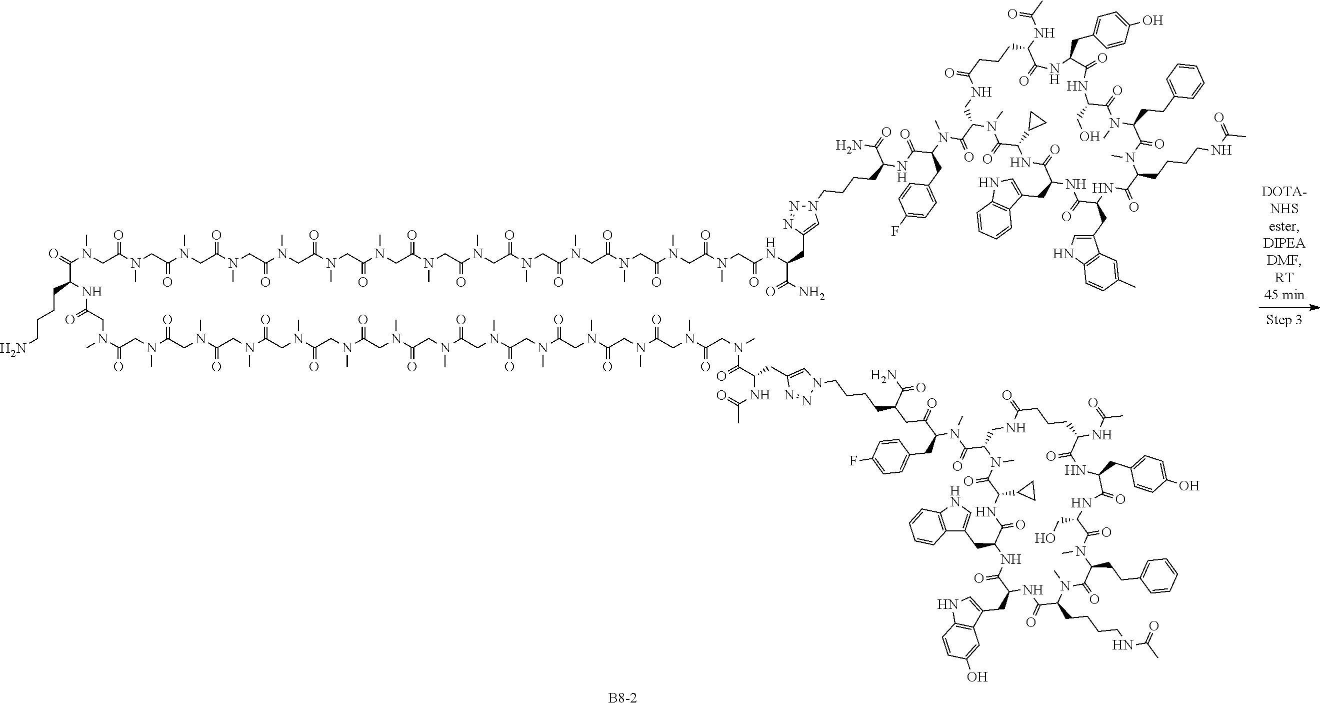

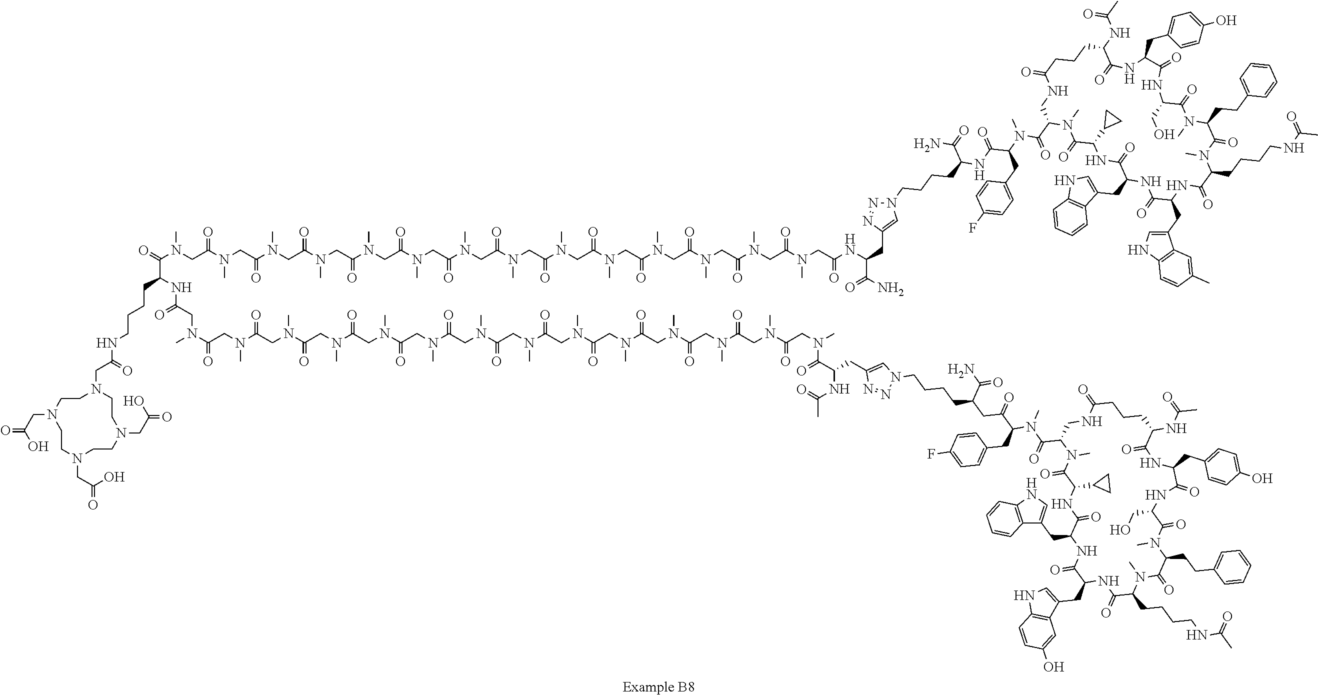

### Step 1. B8-1

Step 1-1: The assembly of the linear peptide was done on the CEM Liberty Prime Synthesizer using commercial Rink amide resin R1 (loading 0.61 mmol/g, 484 mg, 0.300 mmol). The couplings were carried out using the following solutions: Fmoc-amino acid (0.5 M solution DMF, 2.5 mL, 4.2 eq.), DIC (4 M solution in DMF, 0.63 mL, 8.4 eq.), Oxyma Pure® (0.25 M solution in DMF, 5 mL, 4.2 eq.), addition by synthesizer. Fmoc removal was performed using a solution of pyrrolidine (25% in DMF, 1.5 mL), addition by synthesizer.

The amino acids were coupled with a set of different methods.
Method A Fmoc removal: 2×10 min at RT/Coupling: 1×2 min at 105° C.
Method B Fmoc removal: 2×10 min at RT/Coupling: 2×2 min at 105° C.

After the assembly of the linear peptide the resin was washed with DMF (5×) and DCM (5×). The amino acids and coupling methods are summarized in Table 3.3.

TABLE 3.3

| Synthesis | | |
|---|---|---|
| cycle | Amino acid | Method |
| 1 | Fmoc-propargyl-Gly-OH | A |
| 2 | Fmoc-Sar-OH | B |
| 3 | Fmoc-Sar-OH | B |
| 4 | Fmoc-Sar-OH | B |
| 5 | Fmoc-Sar-OH | B |
| 6 | Fmoc-Sar-OH | B |
| 7 | Fmoc-Sar-OH | B |
| 8 | Fmoc-Sar-OH | B |
| 9 | Fmoc-Sar-OH | B |
| 10 | Fmoc-Sar-OH | B |
| 11 | Fmoc-Sar-OH | B |
| 12 | Fmoc-Sar-OH | B |
| 13 | Fmoc-Sar-OH | B |
| 14 | Fmoc-Sar-OH | B |
| 15 | Fmoc-Sar-OH | B |
| 16 | Fmoc-L-Lys(Alloc)-OH | B |
| 17 | Fmoc-Sar-OH | B |
| 18 | Fmoc-Sar-OH | B |
| 19 | Fmoc-Sar-OH | B |
| 20 | Fmoc-Sar-OH | B |
| 21 | Fmoc-Sar-OH | B |
| 22 | Fmoc-Sar-OH | B |
| 23 | Fmoc-Sar-OH | B |
| 24 | Fmoc-Sar-OH | B |
| 25 | Fmoc-Sar-OH | B |
| 26 | Fmoc-Sar-OH | B |
| 27 | Fmoc-Sar-OH | B |
| 28 | Fmoc-Sar-OH | B |
| 29 | Fmoc-Sar-OH | B |
| 30 | Fmoc-Sar-OH | B |
| 31 | Fmoc-propargyl-Gly-OH | B |
| 32 | Acetic acid | B |

Step 1-2: To the resin from Step 1-1 was added TFA/H₂O/DODT/TIS (92.5/2.5/2.5/2.5) (12 mL) and the resin was shaken at RT for 2 hr. The crude peptide was precipitated with cold diethyl ether/heptane (1/1) (120 mL). The suspension was centrifuged, and the solvent was decanted. The product was washed with another portion of cold cold diethyl ether/heptane (1/1) (90 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford B8-1 (151 mg, 61.6 μmol, 20.5% yield) as a white solid. LCMS Method AP-1, t_{R}=2.68 min, [M+H]⁺=2452.3.

### Step 2. B8-2

Step 2-1: B8-1 (15.0 mg, 1 eq., 6.12 μmol) and A74 (20.96 mg, 2 eq., 12.24 μmol) were dissolved in DMSO (3 mL). Then CuSO₄×5 H₂O (0.4 M in water) (4.58 mg, 45.9 μL, 3 eq., 18.35 μmol), followed by sodium (L)-ascorbate (0.4 M in water) (7.272 mg, 91.77 μL, 0.4 molar, 6.0 Eq, 36.71 μmol) were added. The reaction mixture was stirred at RT for 1 hr. Step 2-2: The reaction mixture from Step 2-1 was directly treated with phenylsilane (6.62 mg, 7.54 μL, 10 eq., 61.18 μmol). The reaction mixture was shaken at RT for 10 min while purged with argon. Then, Pd(PPh₃)₄ (1.41 mg, 0.2 eq., 1.224 μmol) was added at RT. The reaction was agitated with argon for 1 hr. The reaction mixture was directly purified, and the product was isolated by preparative HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford B8-2 (26.0 mg, 4.40 μmol, 71.9% yield, TFA salt) as a white solid. LCMS Method AP-1, t_{R}=4.43 min, [M+4H]4+=1449.0.

### Step 3: Example B8

To a clear colorless solution of B8-2 (26 mg, 1 eq., 4.488 μmol, TFA salt) in DMF (2.5 mL) at RT was added DIPEA (5.8 mg, 7.82 μL, 10 eq., 44.88 μmol). The resulting solution was stirred and DOTA-NHS ester (5.12 mg, 1.5 eq., 6.732 μmol, hexafluorophosphate-trifluoroacetate) was added at RT. The reaction mixture was stirred at RT for 45 min. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example B8 (20.4 mg, 3.1 μmol, 69% yield, TFA salt) as a white solid. LCMS Method AP-1, t_{R}=4.31 min, [M+3H]³⁺=2060.3.

Example B8 is made by connecting the linker B8-1 with two cyclic peptides A74 using alkyne and azide [3+2] cycloaddition, followed by Alloc deprotection and DOTA-conjugation. The amino acid composition of B8-1, A74, and B8 is shown in the following table:

| Ex. No. | Sequence |
|---|---|
| B8-1 | NAc-propargyl-Gly-SAR14-Lys(Alloc)-SAR14-propargyl-Gly-NH₂ |
| A74 | NAc-hE-Y-S-NMehF-MeKAc-W(5OH)-W-cProG-NMeDap-NMeF(4F)—K(N3)—NH₂ |
| B8 | NAc-hE-Y-S-NMehF-MeKAc-W(5OH)-W-cProG-NMeDap-NMeF(4F)—K(N3)- |
| | propargyl-Gly-SAR14-K(DOTA)-SAR14-NAc-propargyl-Gly-K(N3)- |
| | NMeF(4F)-NMeDap-cProG-W-W(5OH)-MeKAc-NMehF-S—Y-NAc-hE, |
| | wherein the two K(N3)-propargyl-Gly segments each represent a triazole moiety |
| | formed by alkyne and azide [3 + 2] cycloaddition |

### 3.2 Coupling of NOTA

### 3.2.1 Synthesis of 2,2′-(7-(2-(((S)-5-((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-2-oxoethyl)-1,4,7-triazonane-1,4-diyl)diacetic acid TFA salt (Example B10 (A40 disclosed as SEQ ID NO: 14 and B10 disclosed as SEQ ID NO: 44))

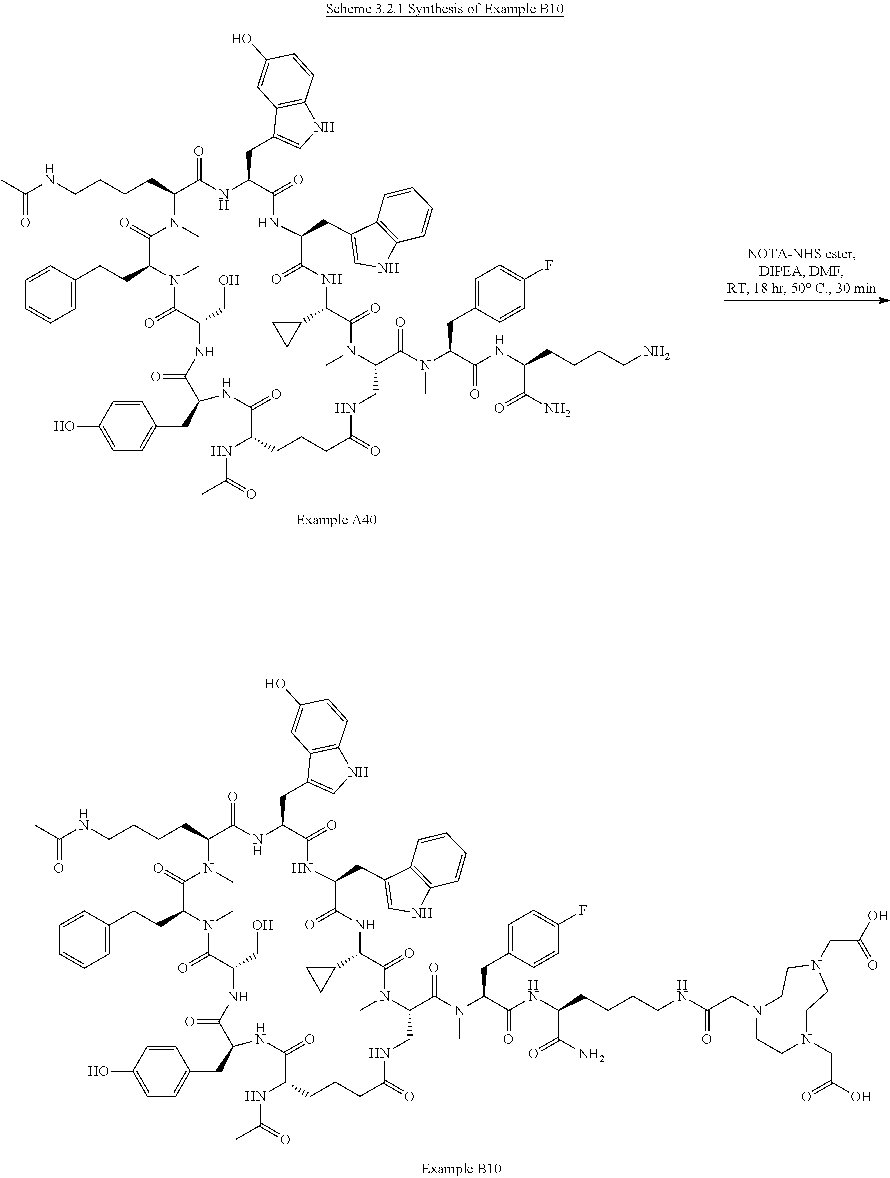

To a clear colorless solution of Example A40 (20 mg, 1 eq., 11.11 μmol, TFA salt) and NOTA-NHS ester (CAS: 1338231-09-6 14.67 mg, 2 eq., 22.21 μmol, hexafluorophosphate-trifluoroacetate) in DMF (1.0 mL) at RT was added DIPEA (7.18 mg, 9.7 μL, 5 eq., 55.53 μmol). The resulting clear solution was stirred at RT for 18 hr. Then additional NOTA-NHS ester (14.67 mg, 2 eq., 22.21 μmol, hexafluorophosphate-trifluoroacetate) was added and the solution was shaken at 50° C. for 30 min. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example B10 (13.6 mg, 5.8 μmol, 52% yield, TFA salt) as a white solid. LCMS Method AP-1, t_{R}=4.04 min, [M+H]⁺=1973.0.

The following examples (for structures, see Table 3.4) were synthesized in analogy to Example B10:

Example B11 was synthesized from Example A43.

TABLE 3.4
Examples synthesized in analogy to Example B10 (Scheme 3.2.1).

| Ex. No. | Structure/Example Name | LCMS |
|---|---|---|
| B11 (SEQ ID NO: 45) | 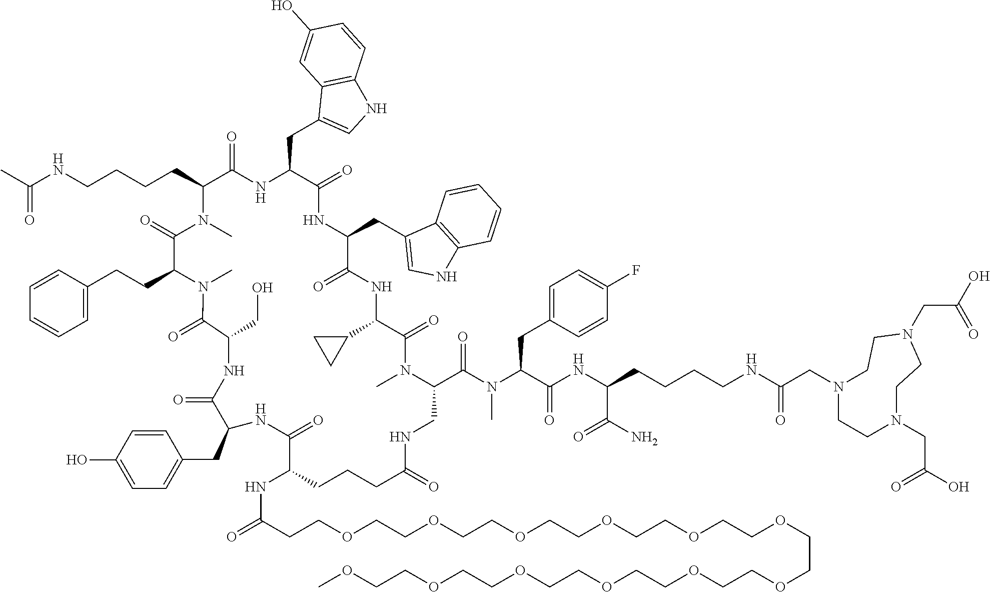 | Method AP-1 t_{R} = 4.43 min [M + H]⁺ = 2501.4 |
| | 2,2′-(7-(2-(((S)-5-((S)-2-((3S,6S,9S,12S,15S,18S,21S,24S,27S)-9-((1H-indol-3- | |
| | yl)methyl)-15-(4-acetamidobutyl)-6-cyclopropyl-12-((5-hydroxy-1H-indol-3- | |
| | yl)methyl)-24-(4-hydroxybenzyl)-21-(hydroxymethyl)-N,4,16,19-tetramethyl- | |
| | 27-(2,5,8,11,14,17,20,23,26,29,32,35-dodecaoxaoctatriacontan-38-amido)- | |
| | 5,8,11,14,17,20,23,26,31-nonaoxo-18-phenethyl-1,4,7,10,13,16,19,22,25- | |
| | nonaazacyclohentriacontane-3-carboxamido)-3-(4- | |
| | fluorophenyl)propanamido)-6-amino-6-oxohexyl)amino)-2-oxoethyl)-1,4,7- | |
| | triazonane-1,4-diyl)diacetic acid TFA salt | |

### 3.3 Coupling of (R)-DOTAGA

### 3.3.1 Synthesis of 2,2′,2″-(10-((R)-4-(((S)-5-((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-1-carboxy-4-oxobutyl)-1,4,7,10-tetraazacyclododecane-1,4,7-triyl)triacetic acid (Example B12 (A40 disclosed as 10 SEQ ID NO: 14 and B12 disclosed as SEQ ID NO: 46))

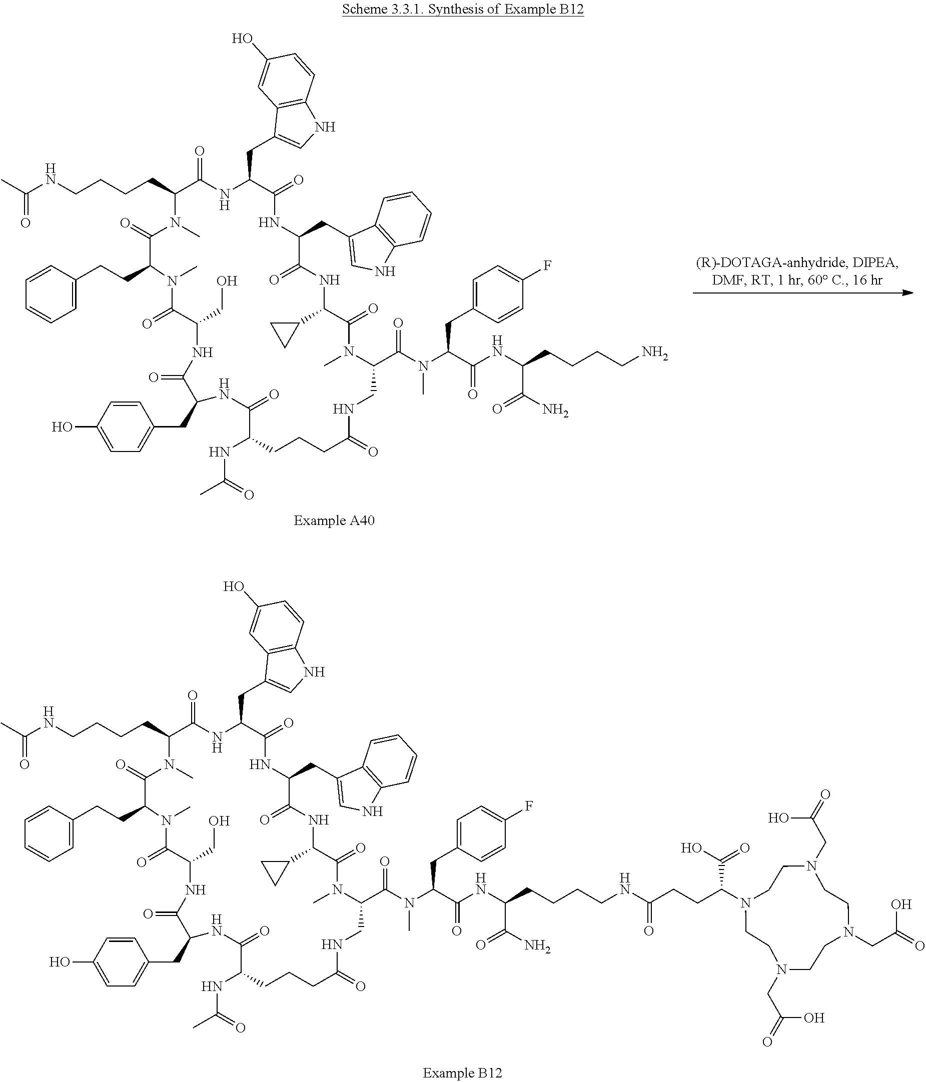

To a clear colorless solution of Example A40 (90 mg, 1 eq., 49.97 μmol, TFA salt) and (R)-DOTAGA anhydride (purchased from CheMatech, 27.49 mg, 1.2 eq., 59.97 μmol) in DMF (4.0 mL) at RT was added DIPEA (38.75 mg, 52.2 μL, 6 eq., 299.8 μmol). The resulting clear solution was stirred at RT for 1 hr. Then additional (R)-DOTAGA anhydride (27.49 mg, 1.2 eq., 59.97 μmol) was added and the solution was shaken at 60° C. for 16 hr. The reaction mixture was precipitated with cold diethyl ether (45 mL) giving a precipitate. The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN) and (Column: XSelect® Peptide CSH C18, OBD™ Prep Column, 130 Å, 5 μm, 30 mm×250 mm; Eluent A: H₂O+0.08% NH₄HCO₃ and eluent B: ACN). Pure fractions were combined and lyophilized to afford Example B12 (52.6 mg, 24.4 μmol, 48.9% yield) as a white solid. LCMS Method AP-1, t_{R}=3.98 min, [M+H]⁺=2146.0.

### 3.4 Coupling of NODAGA

### 3.4.1 Synthesis of 2,2′-(7-(4-(((S)-5-((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-1-carboxy-4-oxobutyl)-1,4,7-triazonane-1,4-diyl)diacetic acid (Example B13 (A40 disclosed as SEQ ID NO: 14 and B13 disclosed as SEQ ID NO: 47))

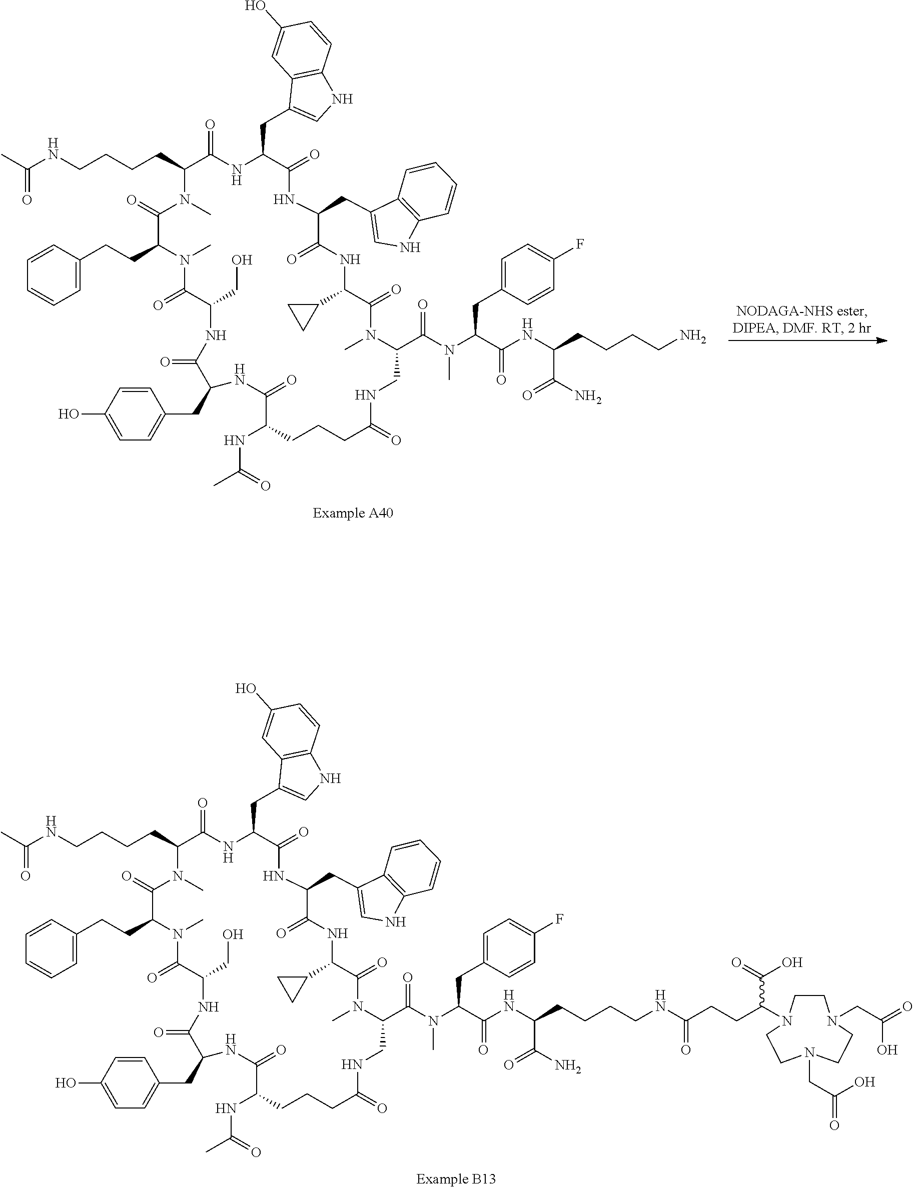

To a clear colorless solution of Example A40 (65 mg, 1 eq., 36.09 μmol, TFA salt) and NODAGA-NHS ester (CAS 1407166-70-4, 34.1 mg, 2 eq., 72.18 μmol) in DMF (4.0 mL) at RT was added DIPEA (46.65 mg, 62.9 μL, 10 eq., 360.9 μmol). The resulting clear solution was stirred at RT for 2 hr. The reaction mixture was precipitated with cold TBME (45 mL) giving a precipitate. The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.08% NH₄HCO₃ and eluent B: ACN). Pure fractions were combined and lyophilized to afford Example B13 (69.3 mg, 33.7 μmol, 93.5% yield) as a white solid. LCMS Method AP-1, t_{R}=4.09 min, [M+H]⁺=2045.0.

### 4 Labelling of Chelators (“C” Examples)

4.1 Labelling with Natural Lutetium (¹⁷⁵Lu)

### 4.1.1 Synthesis of Example C1 (Example B1 Labeled with ¹⁷⁵Lu) (B1 Disclosed as SEQ ID NO: 36 and C1 Disclosed as SEQ ID NO: 48)

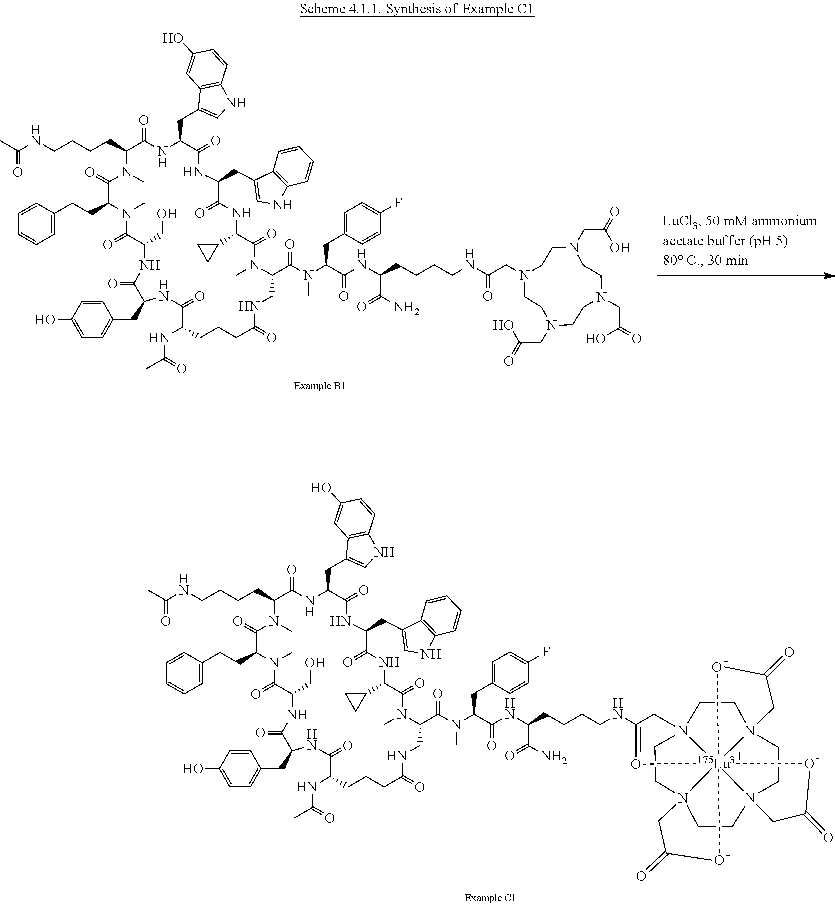

To Example B1 (40 mg, 1 eq., 15.81 μmol, TFA salt) in 50 mM ammonium acetate buffer pH 5 (20.0 mL, 50 mM) was added 100 mM LuCl₃ in ammonium acetate buffer pH 5 (474.4 μL, 100 mM, 3.0 eq., 47.44 μmol). The reaction mixture was stirred at 80° C. for 30 min (clear solution when heated up). The reaction mixture was lyophilized to afford the crude peptide. The product was isolated by preparative HPLC (Column: XBridge BEH C18 OBD Prep Column, 130 Å, 5 μm, 30 mm×150 mm; 50 mL/min; Eluent A: H₂O+0.08% NH₄HCO₃ and eluent B: ACN). Pure fractions were combined and lyophilized to afford Example C1 (29.9 mg, 13 μmol, 83% yield) as a white solid. LCMS Method AP-1, t_{R}=4.18 min, [M+2H]²⁺=2245.9.

The following examples (for structures, see Table 4.1) were synthesized in analogy to Example C1:

Example C2 was synthesized from Example B2.

Example C3 was synthesized from Example B4.

Example C4 was synthesized from Example B8.

Example C31 was synthesized from Example B9.

Example C32 was synthesized from Example B3.

TABLE 4.1
Examples synthesized in analogy to Example C1 (Scheme 4.1.1).

| Ex. No. | Structure/Description | LCMS |
|---|---|---|
| C2 (SEQ ID NO: 49) | 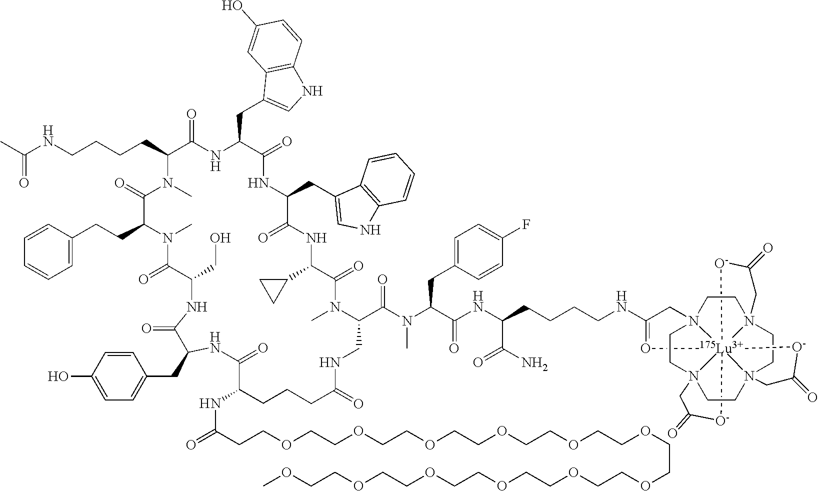 | Method AP-1 t_{R} = 4.58 min [M + H]⁺ = 2774.3 |
| | Example B2 labelled with ¹⁷⁵Lu | |
| C3 (SEQ ID NO: 120) | 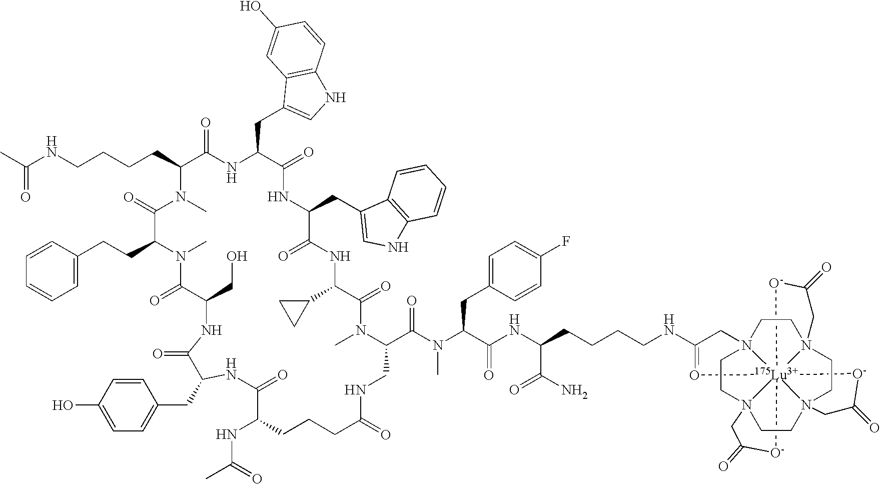 | Method AP-1 t_{R} = 4.34 min [M + H]⁺ = 2246.0 |
| | Example B4 labelled with ¹⁷⁵Lu | |
| C4 | 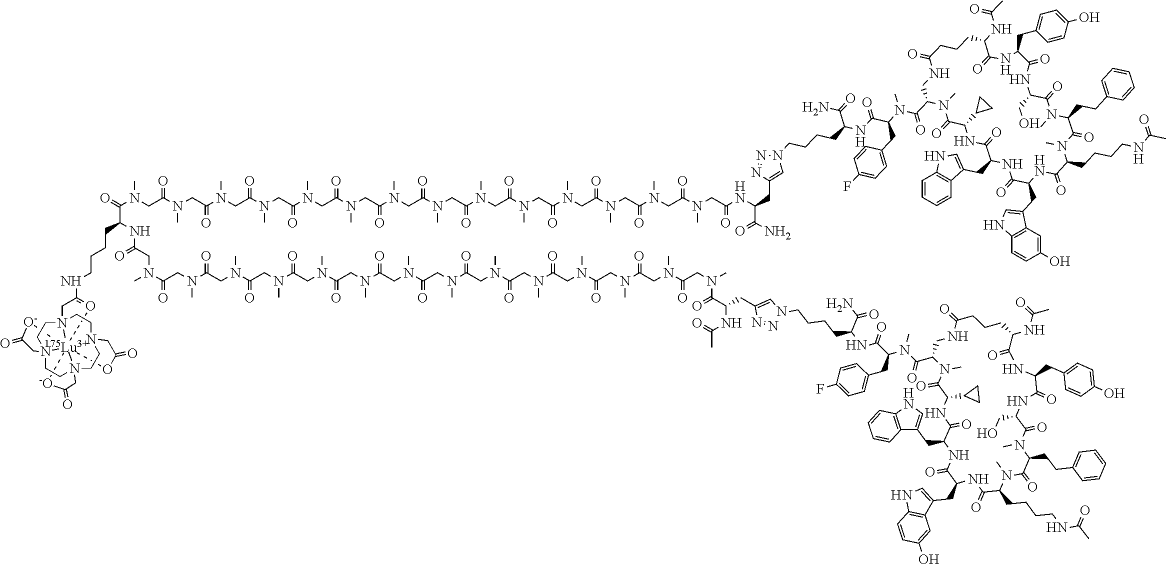 | Method AP1 t_{R} = 4.53 min [M + 3H]³⁺ = 2117.7 |
| | Example B8 labelled with ¹⁷⁵Lu | |
| C31 (SEQ ID NO: 70) | 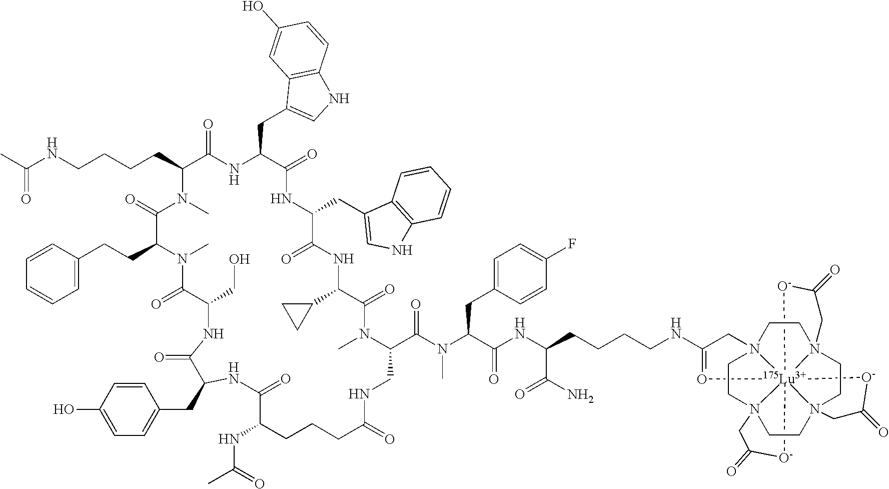 | Method AP1 t_{R} = 4.14 min [M + H]⁺ = 2245.9 |
| | Example B9 labelled with ¹⁷⁵Lu | |
| C32 (SEQ ID NO: 71) | 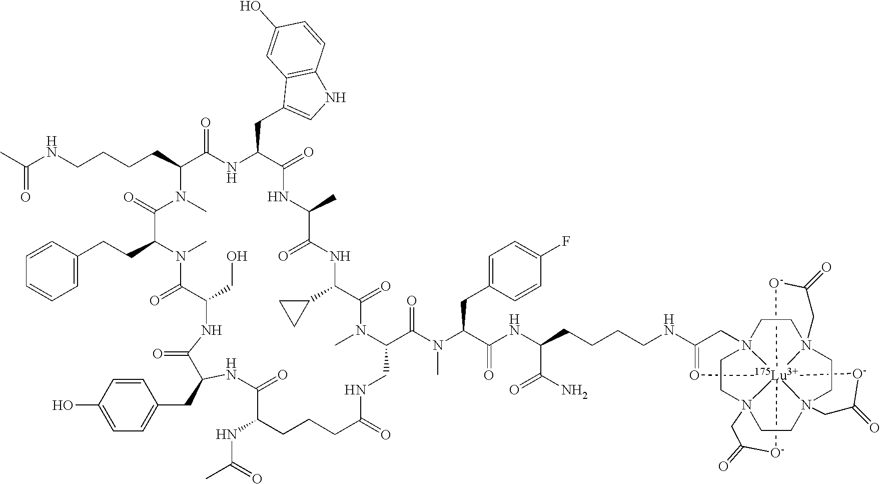 | Method AP1 t_{R} = 3.66 min [M + H]⁺ = 2130.9 |
| | Example B3 Labelled with ¹⁷⁵Lu | |

### 4.1.2 Synthesis of Example C5 (Example A59 Conjugated to DOTA and Labelled with ¹⁷⁵Lu)

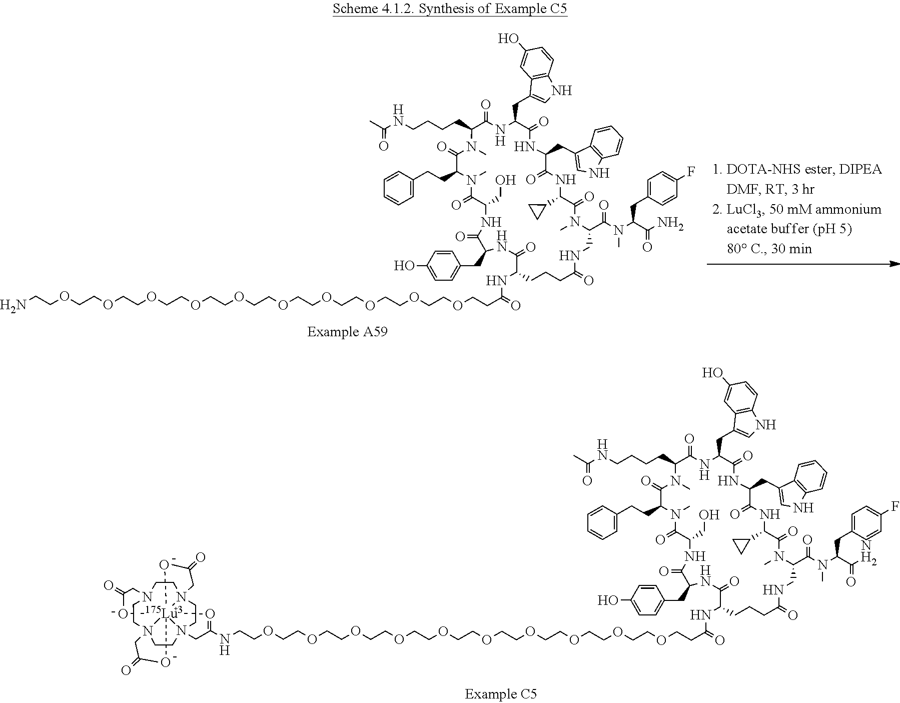

Step 1-1: To a clear solution of the Example A59 (6.0 mg, 1 eq., 2.801 μmol, TFA salt) in DMF (1.5 mL) at RT was added DOTA-NHS ester (3.2 mg, 1.5 eq., 4.201 μmol, hexafluorophosphate-trifluoroacetate), followed by DIPEA (1.81 mg, 2.44 μL, 5 eq., 14.0 μmol). The reaction mixture was stirred at RT for 4 hr.

Step 1-2: To the reaction mixture from Step 1-1, 50 mM ammonium acetate buffer pH 5 (1.5 mL, 50 mM) was added followed by 100 mM LuCl₃ in ammonium acetate buffer pH 5.0 (56 μL, 100 mM, 2 eq., 5.601 μmol). The reaction mixture was stirred at 80° C. for 30 min. The product was isolated by preparative HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example C5 (5.9 mg, 2.1 μmol, 77% yield) as a white solid. LCMS Method AP-1, t_{R}=4.35 min, [M+2H]²⁺=1294.1.

The following examples (for structures, see Table 4.2) were synthesized in analogy to Example C5:

Example C6 was synthesized from Example A41.

Example C7 was synthesized from Example A42.

Example C8 was synthesized from Example A58.

Example C9 was synthesized from Example A47.

Example C10 was synthesized from Example A60.

Example C11 was synthesized from Example A63.

Example C12 was synthesized from Example A61.

Example C13 was synthesized from Example A64.

Example C14 was synthesized from Example A62.

TABLE 4.2
Examples synthesized in analogy to Example C5 (Scheme 4.1.2).

| Ex. No. | Structure/Description | LCMS |
|---|---|---|
| C6 (SEQ ID NO: 50) | 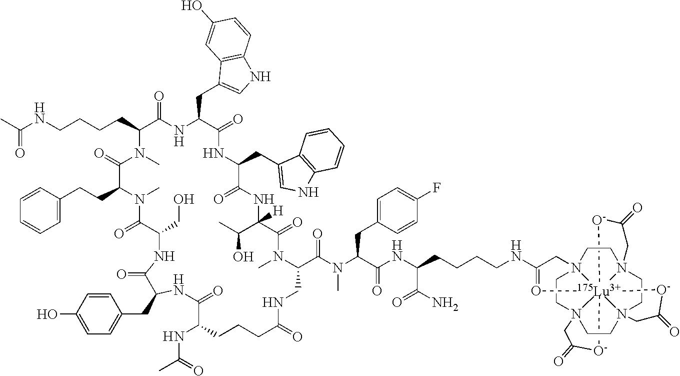 | Method AP1 t_{R} = 3.95 min [M + H]⁺ = 2249.9 |
| | Example A41 conjugated to DOTA and labelled with ¹⁷⁵Lu | |
| C7 | 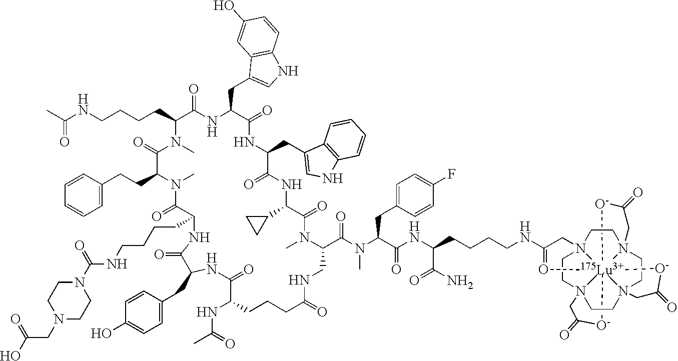 | Method AP-1 t_{R} = 3.92 min [M + 2H]²⁺ = 1229.0 |
| | Example A42 conjugated to DOTA and labelled with ¹⁷⁵Lu | |
| C8 | 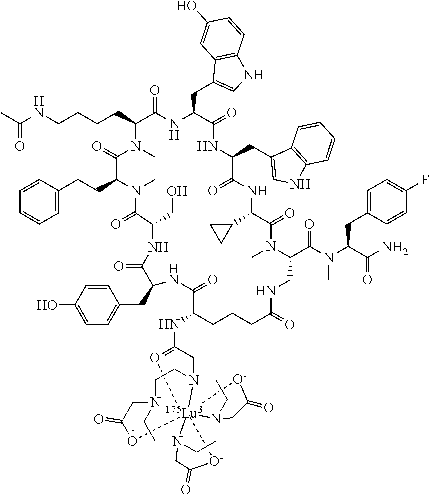 | Method AP-1 t_{R} = 4.20 min [M + H]⁺ = 2075.8 |
| | Example A58 conjugated to DOTA and labelled with ¹⁷⁵Lu | |
| C9 | 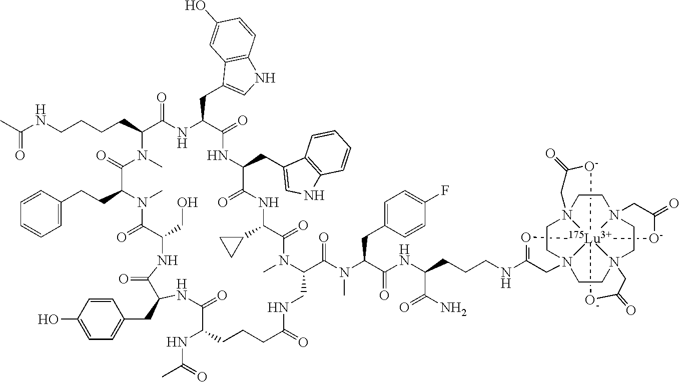 | Method AP-1 t_{R} = 4.17 min [M + H]⁺ = 2231.9 |
| | Example A47 conjugated to DOTA and labelled with ¹⁷⁵Lu | |
| C10 (SEQ ID NO: 51) | 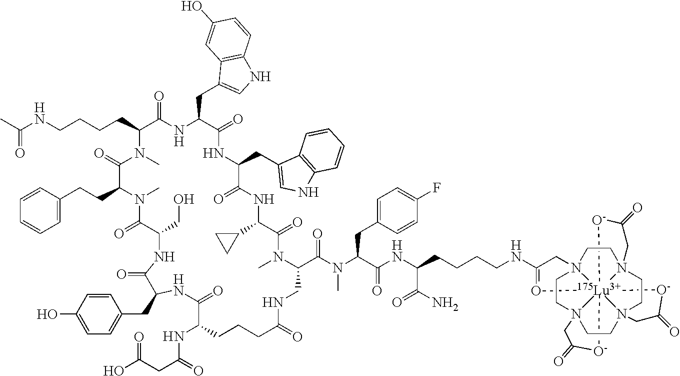 | Method AP-1 t_{R} = 4.13 min [M + H]⁺ = 2289.9 |
| | Example A60 conjugated to DOTA and labelled with ¹⁷⁵Lu | |
| C11 (SEQ ID NO: 52) | 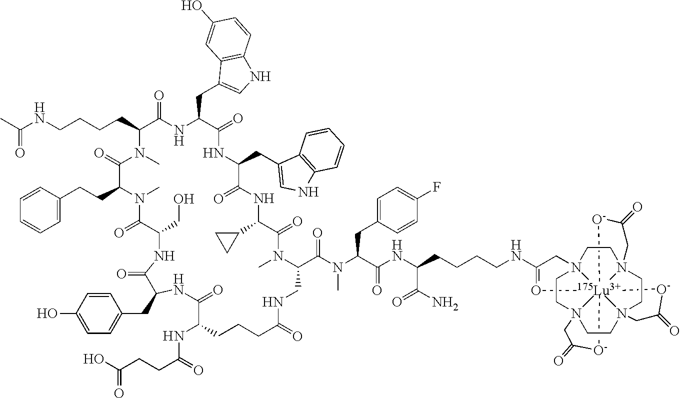 | Method AP-1 t_{R} = 4.14 min [M + 2H]²⁺ = 1152.5 |
| | Example A63 conjugated to DOTA and labelled with ¹⁷⁵Lu | |
| C12 (SEQ ID NO: 53) | 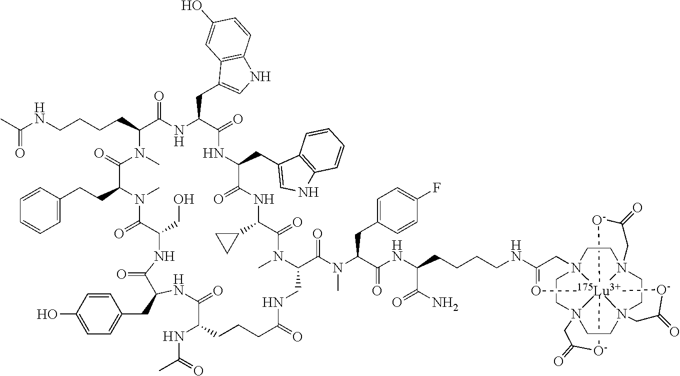 | Method AP-1 t_{R} = 4.22 min [M + H]⁺ = 2246.9 |
| | Example A61 conjugated to DOTA and labelled with ¹⁷⁵Lu | |
| C13 (SEQ ID NO: 54) | 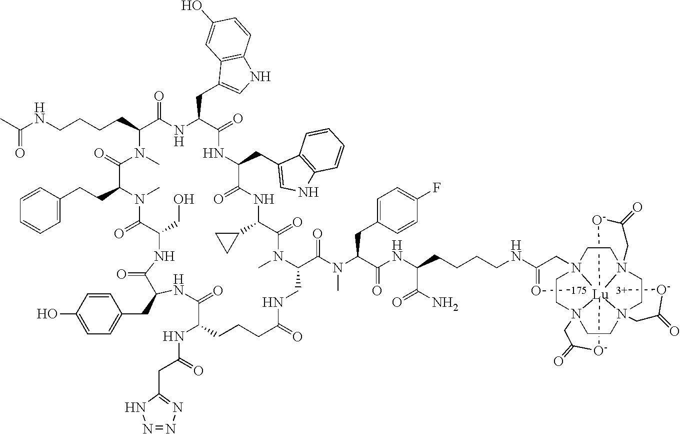 | Method AP-1 t_{R} = 4.15 min [M + 2H]²⁺ = 1157.5 |
| | Example A64 conjugated to DOTA and labelled with ¹⁷⁵Lu | |
| C14 (SEQ ID NO: 55) | 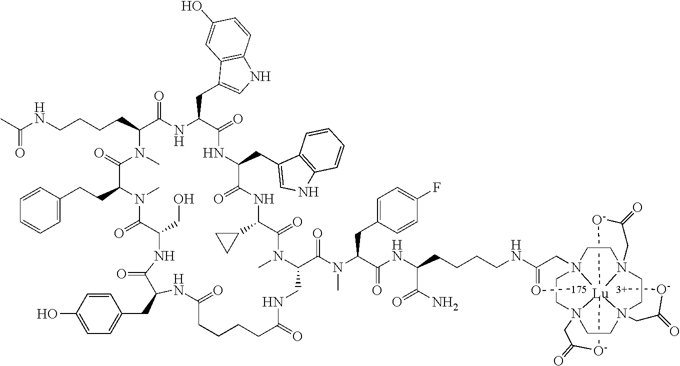 | Method AP-1 t_{R} = 4.20 min [M + H]⁺ = 2188.9 |
| | Example A62 conjugated to DOTA and labelled with ¹⁷⁵Lu | |

### 4.1.3 Synthesis of Example C15 (C15 Disclosed as SEQ ID NO: 56)

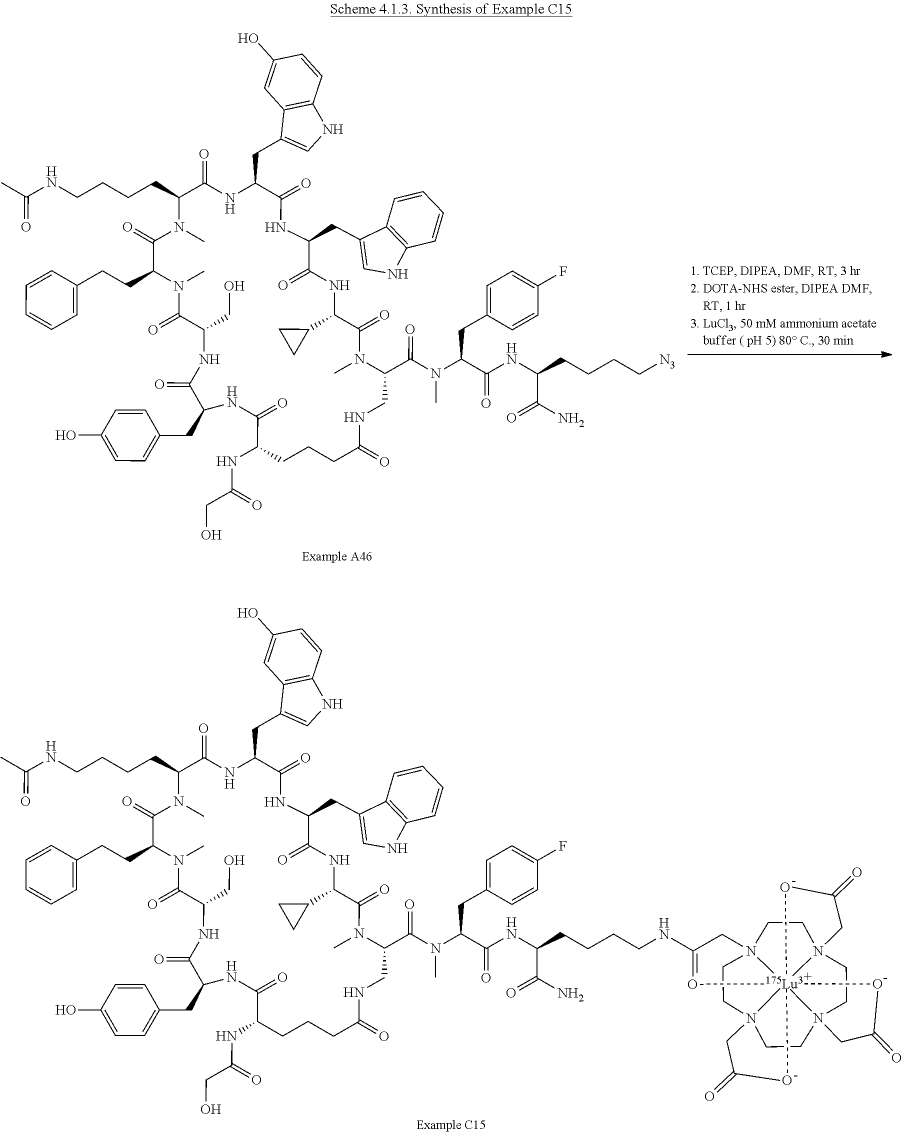

Step 1-1: Example A46 (13.8 mg, 1 eq., 7.982 μmol) was dissolved in DMF (2.00 mL), then TCEP (12.89 mg, 5 eq., 39.91 μmol, HCl salt) and DIPEA (6.19 mg, 8.34 μL, 6 eq., 47.89 μmol) were added. The reaction mixture was shaken at RT for 3 hr.

Step 1-2: To the reaction mixture from Step 1-1 at RT was added DOTA-NHS ester (18.23 mg, 3 eq., 23.95 μmol, hexafluorophosphate-trifluoroacetate) and the reaction mixture was stirred at RT for 1 hr.

Step 1-3: To the reaction mixture from Step 1-2, 50 mM ammonium acetate buffer pH 5 (1 mL, 50 mM) was added followed by 100 mM LuCl₃ in ammonium acetate buffer pH 5.0 (399.1 μL, 100 mM, 5 eq., 39.91 μmol). The reaction mixture was stirred at 80° C. for 30 min. The product was isolated by preparative HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example C15 (9.8 mg, 4.1 μmol, 52% yield) as a white solid. LCMS Method AP-1, t_{R}=4.06 min, [M+2H]²⁺=2261.9.

The following examples (for structures, see Table 4.3) were synthesized in analogy to Example C15:

Example C16 was synthesized from Example A49.

TABLE 4.3
Examples synthesized in analogy to Example C15 (Scheme 4.1.3).

| Ex. No. | Structure/Description | LCMS |
|---|---|---|
| C16 (SEQ ID NO: 57) | 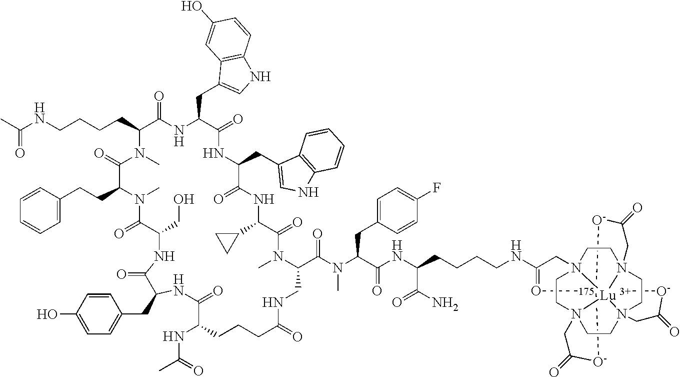 | Method AP-1 t_{R} = 4.10 min [M + 2H]²⁺ = 1124.5 |
| | Example A49 reduced, conjugated to DOTA and labelled with ¹⁷⁵Lu | |

### 4.1.4 Synthesis of Example C17

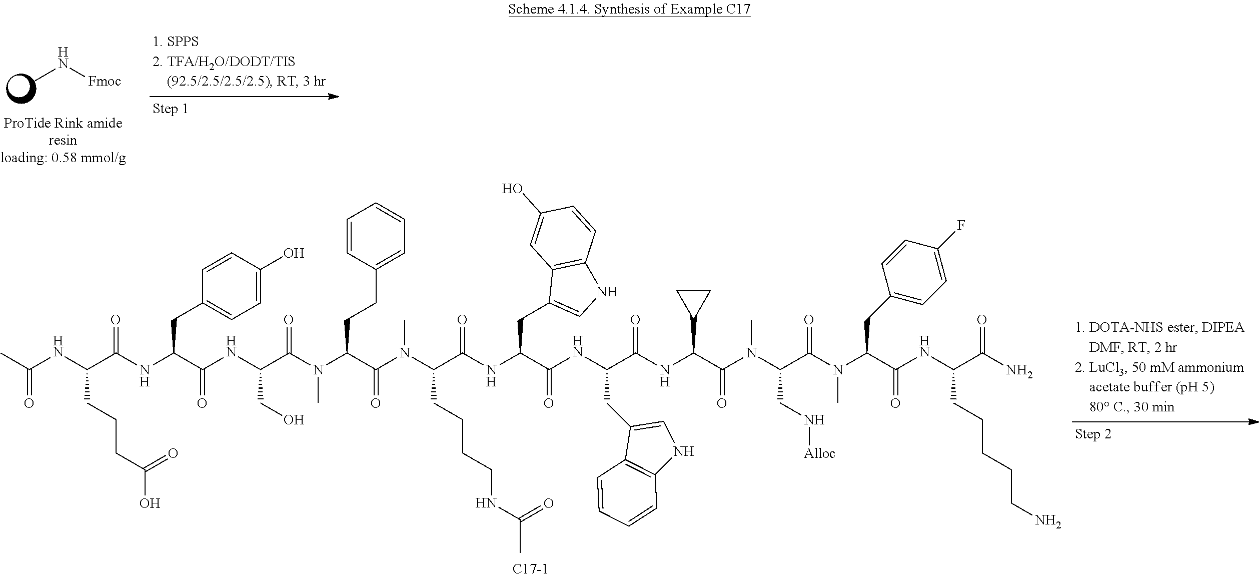

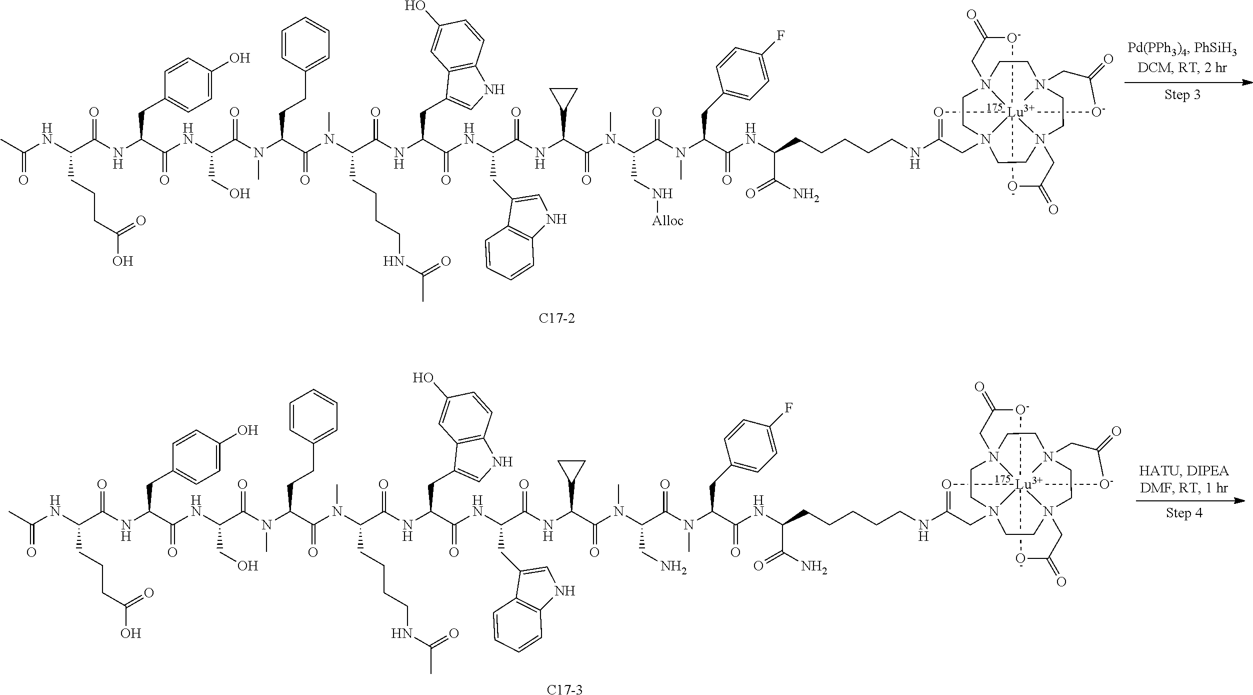

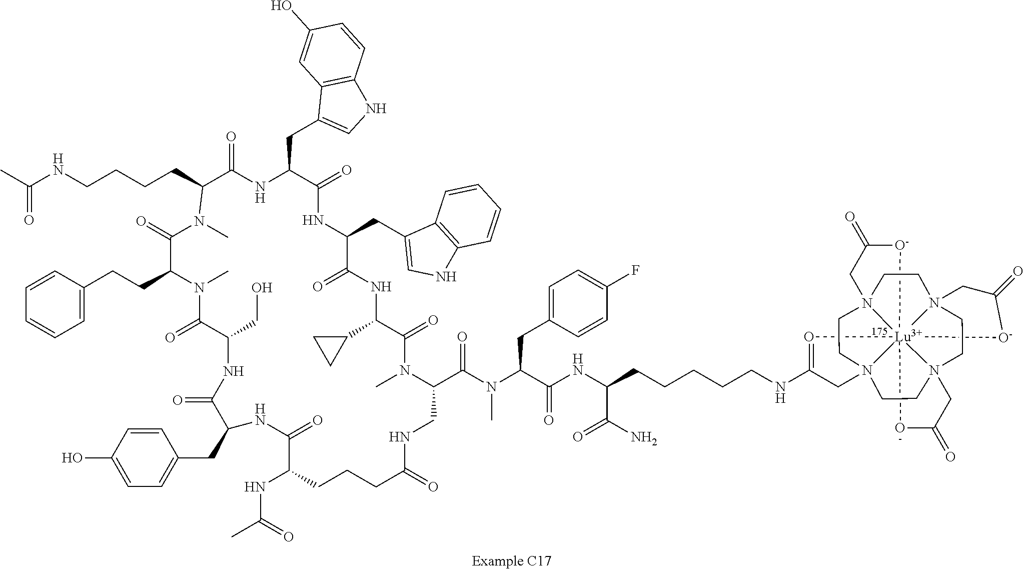

### Step 1. C17-1

Step 1-1: The assembly of the linear peptide was done on the CEM Liberty Blue Synthesizer using Rink amide resin R1 (loading 0.58 mmol/g, 172 mg, 0.100 mmol. The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×2 min at 90° C.
Method B Fmoc removal: 2×1 min at 90° C./Coupling: 2×2 min at 90° C.

After the assembly of the linear peptide the resin was washed with DMF (5×) and DCM (5×). The amino acids and coupling methods are summarized in Table 4.4.

TABLE 4.4

| Synthesis | Amino acid | | |
|---|---|---|---|
| cycle | residue^{a} | Amino acid | Method |
| 1 | A¹⁰* | Fmoc-L-Homo-Lys(Boc)-OH | A |
| 2 | A¹⁰ | Fmoc-L-N-Me-Phe(4F)-OH | A |
| 3 | A⁹ | Fmoc-L-N-Me-Dap(Alloc)-OH | B |
| 4 | A⁸ | Fmoc-L-CyclopropylGly-OH | B |
| 5 | A⁷ | Fmoc-L-Trp(Boc)-OH | A |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | A |
| 11 | A¹ | Fmoc-L-Homo-Glu(OtBu)-OH | A |
| 12 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (4 mL) and the resin was shaken at RT for 3 hr. The crude peptide was precipitated with cold diethyl ether/heptane (1/1) (45 mL). The suspension was centrifuged, and the solvent was decanted. The product was washed with another portion of cold diethyl ether/heptane (1/1) (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford C17-1 (41 mg, 17 μmol, 17% yield, TFA salt) as a white solid. LCMS Method AP-1, t_{R}=4.03 min, [M+H]⁺=1803.9.

### Step 2. C17-2

Step 2-1: To a clear solution of C17-1 (20 mg, 1 eq., 10.43 μmol, TFA salt) in DMF (3 mL) at RT were added DOTA-NHS ester (10 mg, 1.3 eq., 13.56 μmol, hexafluorophosphate-trifluoroacetate), followed by DIPEA (6.7 mg, 9.1 μL, 5 eq., 52.15 μmol). The reaction mixture was stirred at RT for 2 hr.

Step 2-2: To the reaction mixture from Step 2-1, 50 mM ammonium acetate buffer pH 5 (300 μL, 50 mM) was added followed by 100 mM LuCl₃ in ammonium acetate buffer pH 5.0 (140 μL, 100 mM, 1.3 eq., 13.56 μmol). The reaction mixture was stirred at 80° C. for 30 min. The reaction mixture was directly purified, and the product was isolated by preparative HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford C17-2 (17.8 mg, 7.2 μmol, 69% yield) as a white solid. LCMS Method AP-1, t_{R}=4.23 min, [M+2H]²⁺=1181.5.

### Step 3. C17-3

C17-2 (27 mg, 1 eq., 11.44 μmol) (two batches of C17-2 synthesized through different campaigns were combined) was dissolved in DMF (3 mL) and treated with phenylsilane (25 mg, 28 μL, 20 eq., 228.9 μmol). The reaction mixture was shaken at RT for 10 min while purged with argon. A clear solution of Pd(PPh₃)₄ (1.3 mg, 0.1 eq., 1.14 μmol) in DCM (1 mL) was added at RT. The reaction was agitated with argon for 2 hr. The reaction mixture was directly purified, and the product was isolated by preparative HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford C17-3 (18.0 mg, 7.5 μmol, 68% yield, TFA salt) as a white solid. LCMS Method AP-1, t_{R}=3.57 min, [M+2H]²⁺=1139.5.

### Step 4. Example C17

C17-3 (18 mg, 1 eq., 7.5 μmol, TFA salt) was dissolved in DMF (8 mL). Then HATU (2.9 mg, 1 eq., 7.5 μmol), followed by DIPEA (4.9 mg, 6.6 μL, 5 eq., 37.5 μmol) were added. The reaction mixture was stirred at RT for 1 hr. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example C17 (8 mg, 5.2 μmol, 20% yield) as a white solid. LCMS Method AP-1, t_{R}=4.20 min, [M+H]⁺=1130.5.

### 4.1.5 Synthesis of Example C18 (C18 Disclosed as SEQ ID NO: 58)

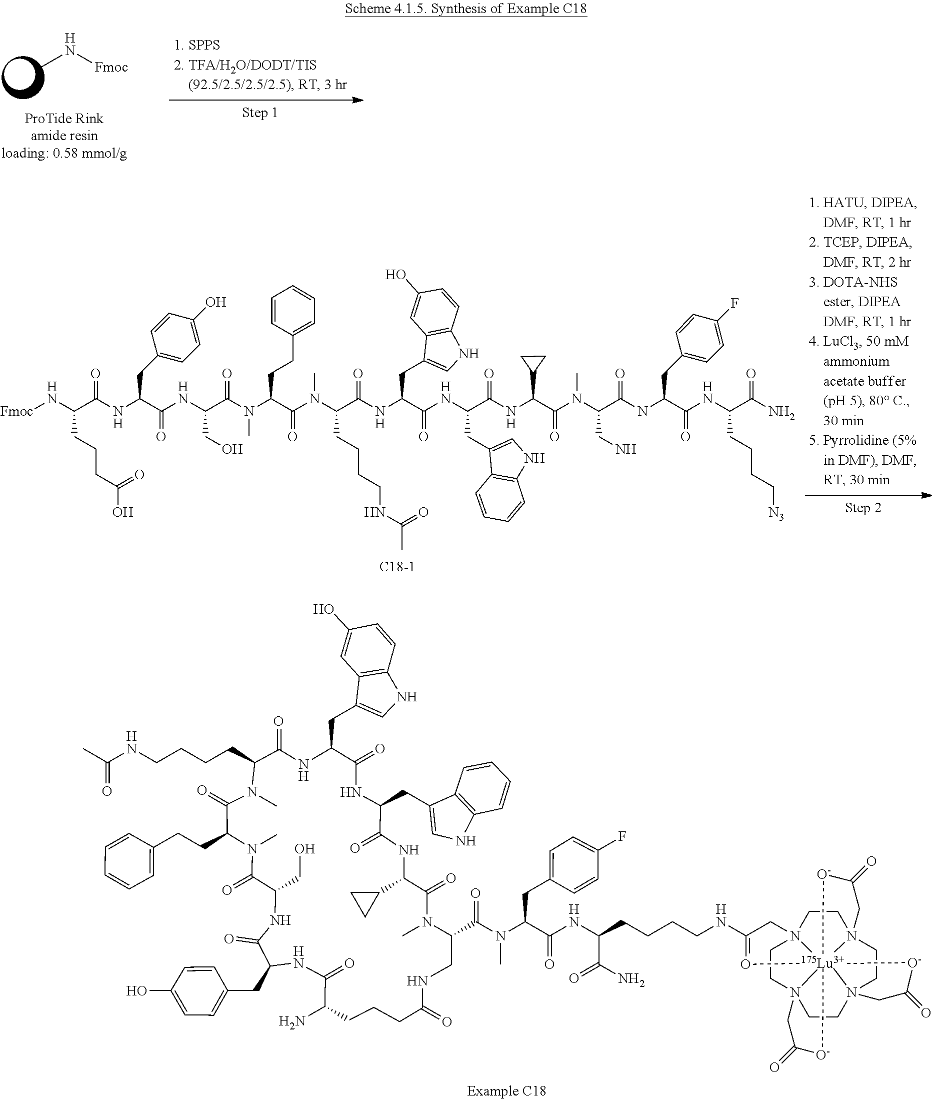

### Step 1. C18-1

Step 1-1: The assembly of the linear peptide was done on the CEM Liberty Blue Synthesizer using Rink amide resin R1 (loading 0.58 mmol/g, 172 mg, 0.100 mmol). The couplings were carried out using the following solutions: Fmoc-amino acid (0.2 M solution DMF, 2.5 mL, 5 eq.), DIC (1 M solution in DMF, 1 mL, 10 eq.), Oxyma Pure® (1 M solution in DMF+0.1 M DIPEA, 0.5 mL, 5 eq.), addition by synthesizer. Fmoc removal was performed using a solution of pyrrolidine (5% in DMF, 4 mL), addition by synthesizer.

The amino acids were coupled with a set of different methods.
Method A Fmoc removal: 1×1 min at 90° C./Coupling: 1×2 min at 90° C.
Method B Fmoc removal: 2×1 min at 90° C./Coupling: 2×2 min at 90° C.

After the assembly of the linear peptide the resin was washed with DMF (5×) and DCM (5×). The amino acids and coupling methods are summarized in in Table 4.5.

TABLE 4.5

| Synthesis | Amino acid | | |
|---|---|---|---|
| cycle | residue^{a} | Amino acid | Method |
| 1 | A¹⁰* | Fmoc-L-Lys(N3)-OH | A |
| 2 | A¹⁰ | Fmoc-L-N-Me-Phe(4F)-OH | A |
| 3 | A⁹ | Fmoc-L-N-Me-Dap(Boc)-OH | B |
| 4 | A⁸ | Fmoc-L-CyclopropylGly-OH | B |
| 5 | A⁷ | Fmoc-L-Trp(Boc)-OH | A |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | A |
| 11 | A¹ | Fmoc-L-Homo-Glu(OtBu)-OH | A |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (4 mL) and the resin was shaken at RT for 30 min. The crude peptide was precipitated with cold diethyl ether/heptane (1/1) (45 mL). The suspension was centrifuged, and the solvent was decanted. The product was washed with another portion of cold diethyl ether (30 mL). The suspension was centrifuged, and the solvent was decanted. The cleavage procedure was repeated once. The combined crude peptide was dried at RT under vacuo, and the product was isolated by preparative RP HPLC (Column: XBridge C18 OBD™ Prep Column, 130 Å, 5 μm, 50 mm×100 mm; Eluent A: H₂O+0.1% TFA and eluent B: ACN). Pure fractions were combined and lyophilized to afford C18-1 (15 mg, 7.407 μmol, 7.4% yield, TFA salt) as a white solid. LCMS Method AP-1, t_{R}=4.96 min, [M+H]⁺=1911.9.

### Step 2. Example C18

Step 2-1: C18-1 (15 mg, 1 eq., 7.407 μmol, TFA salt) was dissolved in DMF (3 mL). Then HATU (2.82 mg, 1 eq., 7.407 μmol), followed by DIPEA (4.8 mg, 6.45 μL, 5 eq., 37.04 μmol) were added. The reaction mixture was stirred at RT for 1 hr.

Step 2-2: To the reaction mixture from Step 2-1 was added TCEP (8.49 mg, 4 eq., 29.63 μmol, HCl salt). The reaction mixture was shaken at RT for 16 hr.

Step 2-3: To the reaction mixture from Step 2-2 was added DOTA-NHS ester (16.92 mg, 3 eq., 22.22 μmol, hexafluorophosphate-trifluoroacetate), followed by DIPEA (4.8 mg, 6.45 μL, 5 eq., 37.04 μmol). The reaction mixture was stirred at RT for 1 hr.

Step 2-4: To the reaction mixture from Step 2-3, 50 mM ammonium acetate buffer pH 5 (250 μL, 50 mM) was added followed by 100 mM LuCl₃ in ammonium acetate buffer pH 5.0 (222 μL, 100 mM, 3 eq., 22.22 μmol). The reaction mixture was stirred at 80° C. for 30 min.

Step 2-5: To the reaction mixture from Step 2-4 at RT, was added pyrrolidine (5% solution in DMF) (300 μL, 1 eq., 7.407 μmol). The reaction mixture was stirred at RT for 30 min. The product was isolated by preparative RP HPLC (Column: XBridge C18 OBD™ Prep Column, 130 Å, 5 μm, 50 mm×100 mm; Eluent A: H₂O+0.1% TFA and eluent B: ACN). Pure fractions were combined and lyophilized to afford Example C18 (6.6 mg, 2.6 μmol, 35% yield, TFA salt) as a white solid. LCMS Method AP-1, t_{R}=4.35 min, [M+H]⁺=2203.9.

### 4.1.6 Synthesis of Example C19

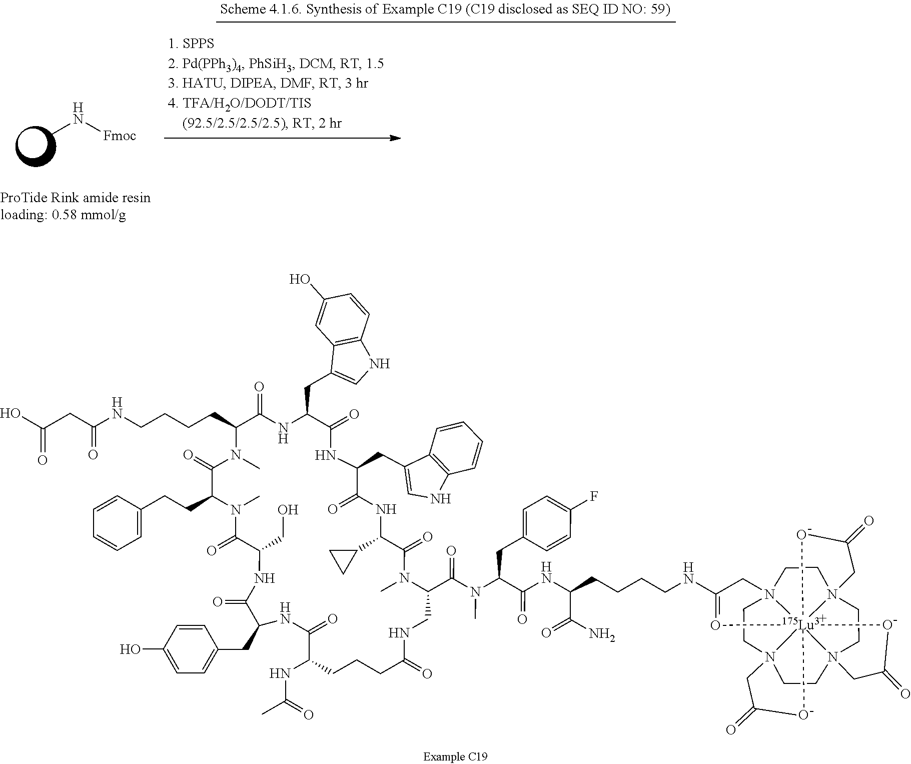

Step 1-1: The assembly of the linear peptide was done on the CEM Liberty Prime Synthesizer and by manual coupling using commercial Rink amide resin R1 (loading 0.58 mmol/g, 172 mg, 0.100 mmol). The couplings were carried out using the following solutions: Fmoc-amino acid (0.5 M solution DMF, 1 mL, 5 eq.), DIC (2 M solution in DMF, 0.5 mL, 10 eq.), Oxyma Pure® (0.25 M solution in DMF, 2 mL, 5 eq.), addition by synthesizer. Fmoc removal was performed using a solution of pyrrolidine (25% in DMF, 0.75 mL), addition by synthesizer.

The amino acids were coupled with a set of different methods.
Method A Fmoc removal: 1×40 sec at 110° C./Coupling: 1×2 min at 105° C.
Method B Fmoc removal: 2×10 min at RT/Coupling: 2×2 min at 105° C.
Method C Fmoc removal: 2×10 min at RT/Coupling: 1×2 min at 105° C.

Fmoc deprotection (after Synthesis cycle 3 and 6) was performed using 25% pyrrolidine in DMF (0.75 mL), addition by synthesizer, 1×40 sec at 110° C.

### Method D (Manual Coupling)

The resin was treated with a pre-activated solution of Fmoc-L-CyclopropylGly-OH (37.1 mg, 1.2 eq., 120 μmol), HATU (57.0 mg, 1.5 eq., 150 μmol) and DIPEA (38.8 mg, 52.3 μL, 3 eq., 300 μmol) in DMF (4 mL) and mixed at RT for 5 hr.

### Method E (Manual Coupling)

The resin was treated with a pre-activated solution of BB3 (63.0 mg, 1.2 eq., 120 μmol), HATU (57.0 mg, 1.5 eq., 150 μmol) and DIPEA (38.8 mg, 52.3 μL, 3 eq., 300 μmol) in DMF (4 mL) and mixed at RT for 5 hr.

After the assembly of the linear peptide the resin was washed with DMF (5×) and DCM (5×). The amino acids and coupling methods are summarized in Table 4.6.

TABLE 4.6

| | Amino | | |
|---|---|---|---|
| Synthesis | acid | | |
| cycle | residue^{a} | Amino acid | Method |
| 1 | A¹⁰* | BB5 | A |
| 2 | A¹⁰ | Fmoc-L-N-Me-Phe(4F)-OH | A |
| 3 | A⁹ | Fmoc-L-N-Me-Dap(Alloc)-OH | B + Fmoc |
| | | | deprotection |
| 4 | A⁸ | Fmoc-L-CyclopropylGly-OH | D (Manual |
| | | | coupling) |
| 5 | A⁷ | Fmoc-L-Trp(Boc)-OH | A |
| 6 | A⁶ | Fmoc-L-Trp(5-OH)-OH | A + Fmoc |
| | | | deprotection |
| 7 | A⁵ | BB3 | E (Manual |
| | | | coupling) |
| 8 | A⁴ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | C |
| 11 | A¹ | Fmoc-L-Homo-Glu(OAlloc)-OH | A |
| 12 | A¹* | Acetic acid | A |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 100 μmol) was suspended with DCM (5 mL) and treated with phenylsilane (216 mg, 248 μL, 20 eq., 2 mmol). The suspension was shaken at RT for 5 min while purged with argon. Then Pd(PPh₃)₄ (23.1 mg, 0.2 eq., 20 μmol) was added at RT. The reaction was agitated with argon for 1.5 hr. The resin was drained and washed with DMF (5×) and DCM (5×).

Step 1-3: The resin from Step 1-2 (calculated with 100 μmol) was suspended with DMF (10 mL). Then HATU (57 mg, 1.5 eq., 150 μmol), followed by DIPEA (38.8 mg, 52.3 μL, 3 eq., 300 μmol) were added. The reaction mixture was stirred at RT for 3 hr. The resin was drained and washed with DMF (5×) and DCM (5×).

Step 1-4: To the resin from Step 1-3 (calculated with 100 μmol) was added TFA/H₂O/DODT/TIS (92.5/2.5/2.5/2.5) (4 mL) and the resin was shaken at RT for 2 hr. The crude peptide was precipitated with cold diethyl ether (40 mL) giving a precipitate. The suspension was centrifuged, and the solvent was decanted. The product was washed with another portion of cold diethyl ether (30 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN) followed by preparative RP HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example C19 (0.7 mg, 0.3 μmol, 0.3% yield) as a white solid. LCMS Method AP-1, t_{R}=4.03 min, [M+2H]²⁺=1145.5.

### 4.1.7 Synthesis of Example C20 (Example B5 Labelled with ¹⁷⁵Lu) (A65 Disclosed as SEQ ID NO: 26 and C20 Disclosed as SEQ ID NO: 60)

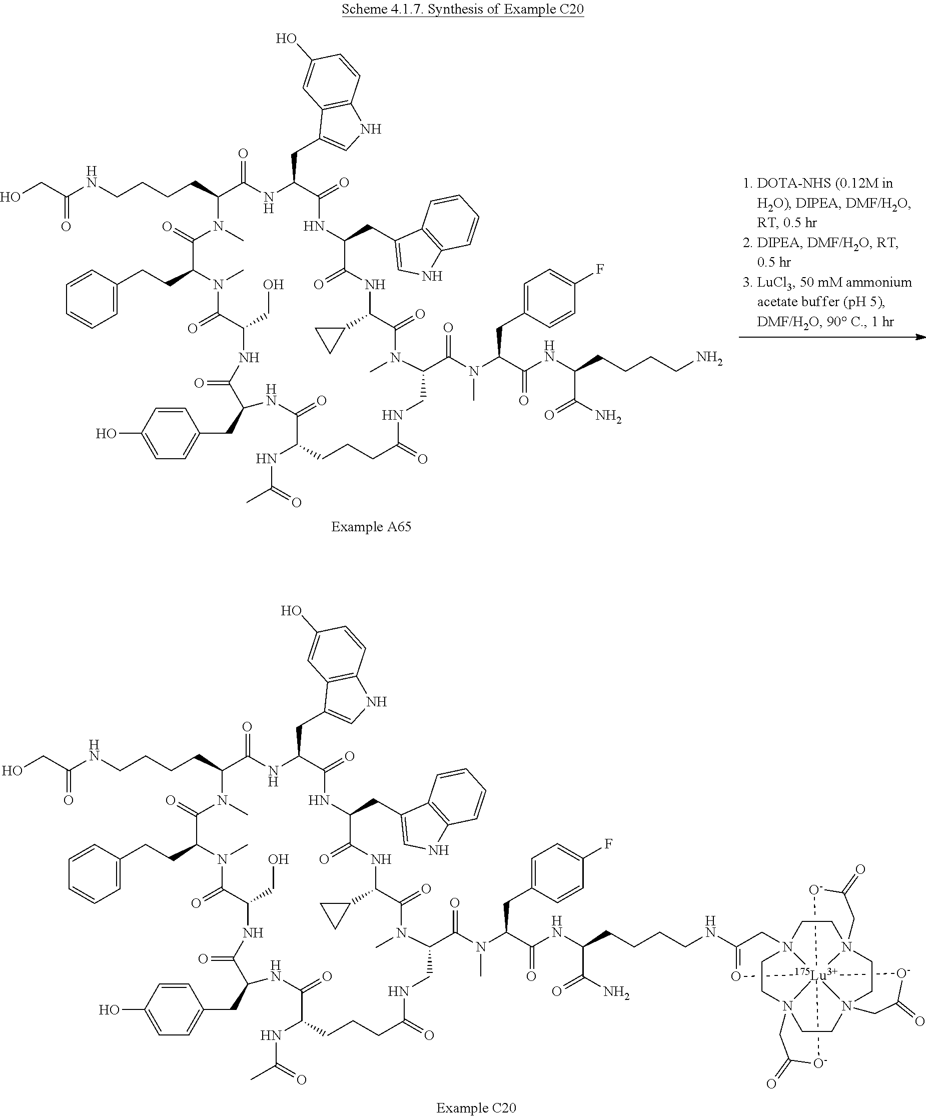

Step 1-1: Example A65 (40 mg, 1 eq., 22.0 μmol, TFA salt) was dissolved in DMF (1.10 mL), then DIPEA (25.6 mg, 35 μL, 9 eq., 198 μmol) and a 0.12 M solution of DOTA-NHS-ester (prepared as described in Section 3.1.2) (1.10 mL, 6 eq., 132 μmol) were added at 0° C. The reaction mixture was stirred at RT for 0.5 hr.

Step 1-2: DIPEA (25.6 mg, 35 μL, 9 eq., 198 μmol) was added to the mixture from Step 1-1. Then the reaction mixture was stirred at RT for an additional 0.5 hr.

Step 1-3: To the mixture from Step 1-2, was added a solution of 50 mM LuCl₃ in ammonium acetate buffer (4.83 ml, 11 eq., 242 μmol, pH 5) and the mixture was stirred at 90° C. for 1 hr. The product was isolated by preparative RP HPLC (Column: Kinetex® EVO C18, OBD™ Prep Column, 130 Å, 5 μm, 30 mm×150 mm; Eluent A: H₂O+1.0% AcOH and eluent B: ACN+1.0% AcOH). Pure fractions were combined and lyophilized to afford Example C20 (26.6 mg, 11.5 μmol, 52% yield, AcOH salt) as a white solid. LCMS Method AP-3, t_{R}=4.20 min, [M+2H]²⁺=1131.4.

The following examples (for structures, see Table 4.7) were synthesized in analogy to Example C20:

Example C21 was synthesized from Example B6.

Example C22 was synthesized from Example B7.

Example C23 was synthesized from Example A68.

Example C24 was synthesized from Example A69.

Example C25 was synthesized from Example A70.

Example C26 was synthesized from Example A71.

Example C27 was synthesized from Example A72.

Example C28 was synthesized from Example A73.

TABLE 4.7
Examples synthesized in analogy to Example C20 (Scheme 4.1.7).

| Ex. No. | Structure/Example Name | LCMS |
|---|---|---|
| C21 (SEQ ID NO: 61) | 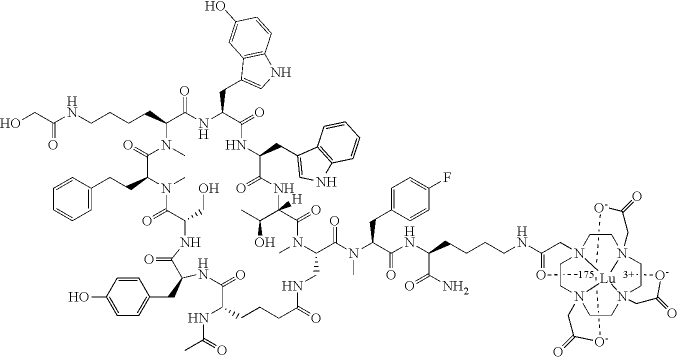 | Method AP-3 t_{R} = 3.90 min [M + 2H]²⁺ = 1133.6 |
| | Example B6 labelled with ¹⁷⁵Lu AcOH salt | |
| C22 (SEQ ID NO: 62) | 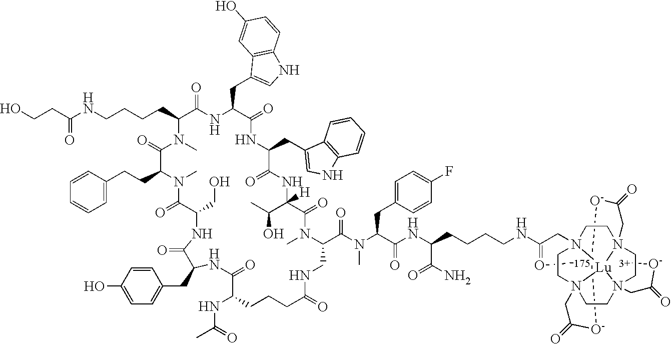 | Method AP-3 t_{R} = 3.89 min [M + 2H]²⁺ = 1140.6 |
| | Example B7 labelled with ¹⁷⁵Lu AcOH salt | |
| C23 (SEQ ID NO: 63) | 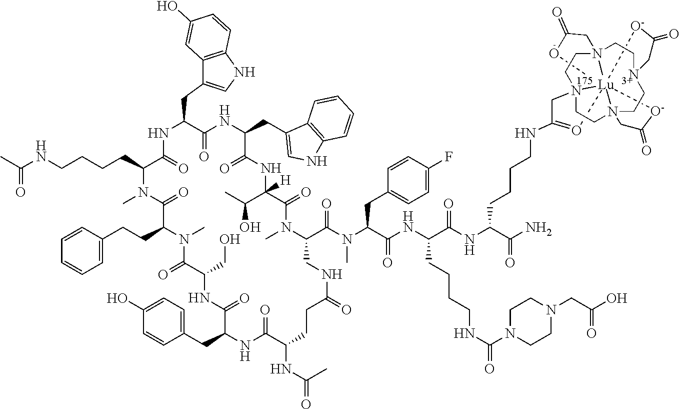 | Method AP-3 t_{R} = 3.66 min [M + 2H]²⁺ = 1267.6 |
| | Example A68 conjugated to DOTA and labelled with ¹⁷⁵Lu AcOH salt | |
| C24 (SEQ ID NO: 64) | 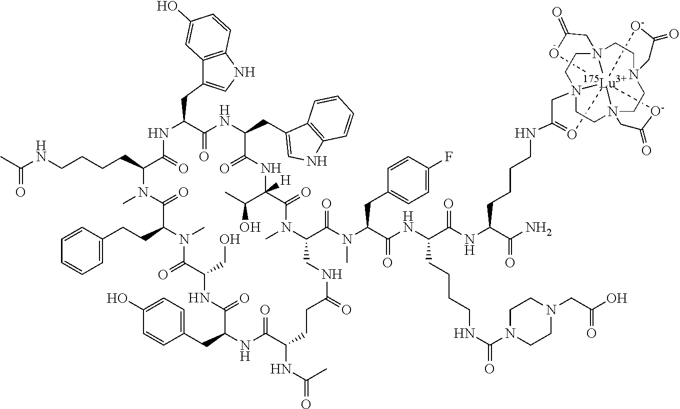 | Method AP-3 t_{R} = 3.61 min [M + 2H]²⁺ = 1267.6 |
| | Example A69 conjugated to DOTA and labelled with ¹⁷⁵Lu AcOH salt | |
| C25 (SEQ ID NO: 65) | 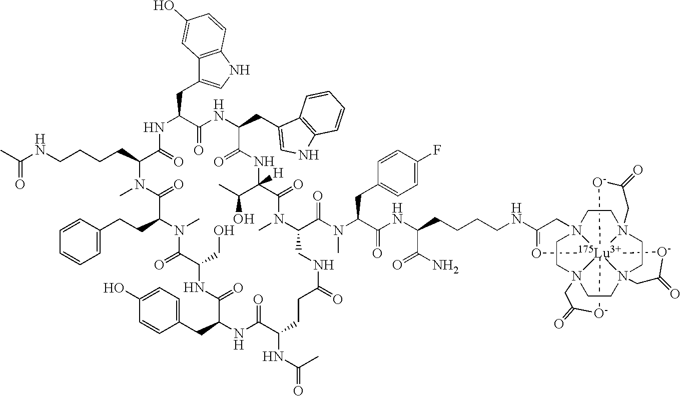 | Method AP-3 t_{R} = 4.15 min [M + 2H]²⁺ = 1118.4 |
| | Example A70 conjugated to DOTA and labelled with ¹⁷⁵Lu AcOH salt | |
| C26 | 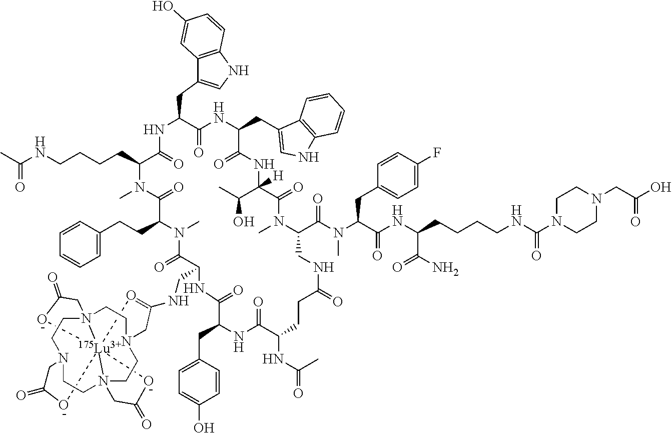 | Method AP-3 t_{R} = 3.60 min [M + 2H]²⁺ = 1203.0 |
| | Example A71 conjugated to DOTA and labelled with ¹⁷⁵Lu AcOH salt | |
| C27 (SEQ ID NO: 66) | 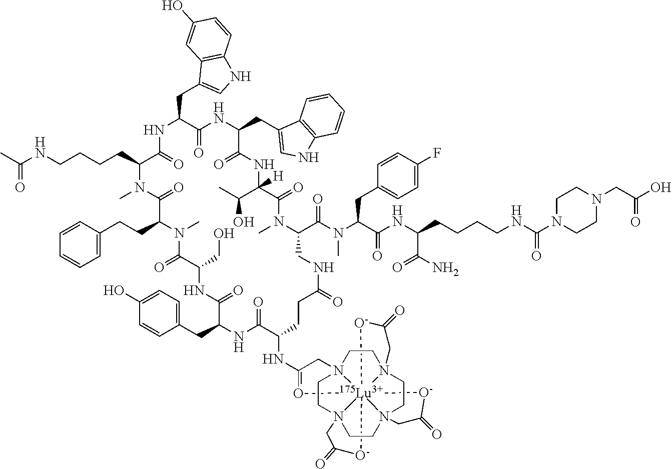 | Method AP-3 t_{R} = 3.71 min [M + 2H]²⁺ = 1182.5 |
| | Example A72 conjugated to DOTA and labelled with ¹⁷⁵LuAcOH salt | |
| C28 (SEQ ID NO: 67) | 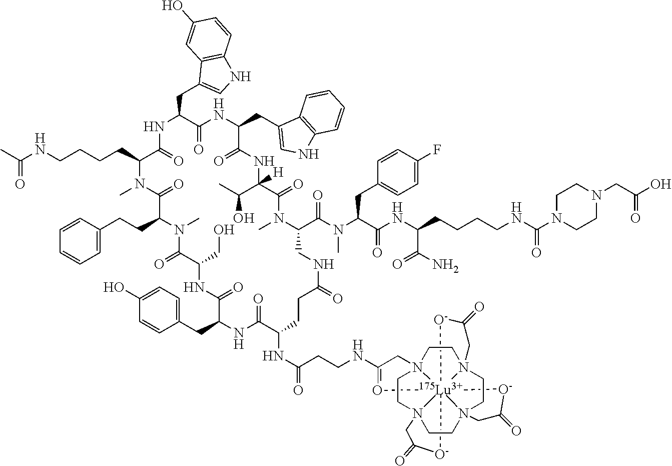 | Method AP-3 t_{R} = 3.69 min [M + 2H]²⁺ = 1218.1 |
| | Example A73 conjugated to DOTA and labelled with ¹⁷⁵Lu AcO salt | |

4.2 Labelling with Natural Gallium (Ga)

### 4.2.1 Synthesis of Example C29 (Example B1 Labeled with Ga) (B1 Disclosed as SEQ ID NO: 36 and C29 Disclosed as SEQ ID NO: 68)

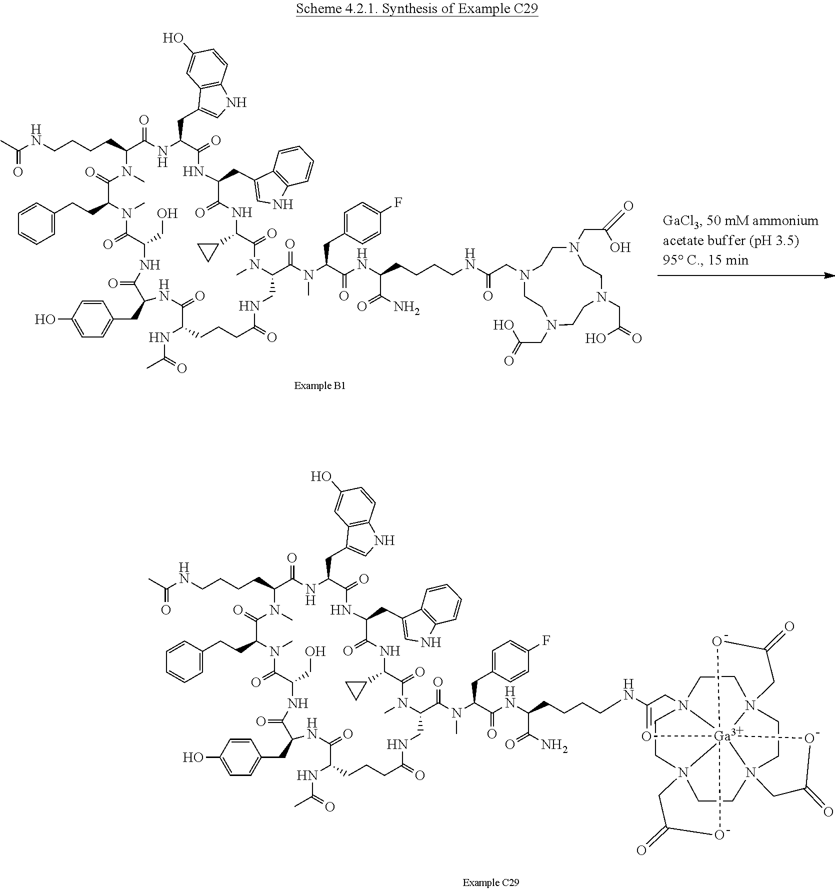

To Example B1 (30.0 mg, 1 eq., 11.86 μmol, TFA salt) in ammonium acetate buffer pH 3.5 (10.0 mL, 50 mM) was added 1 M GaCl₃ in ammonium acetate buffer pH 3.5 (23.72 μL, 1.0 M, 2.0 eq., 23.72 μmol). The reaction mixture was stirred at 95° C. for 15 min (clear solution when heated up). The reaction mixture was concentrated to dryness in vacuo to afford the crude peptide. The product was isolated by preparative HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example C29 (22.2 mg, 10 μmol, 87% yield) as a white solid. LCMS Method AP-1, t_{R}=3.93 min, [M+2H]²⁺=1070.5.

### 4.2.2 Synthesis of Example C33 (Example B12 Labeled with Ga) (B12 Disclosed as SEQ ID NO: 46 and C33 Disclosed as SEQ ID NO: 72)

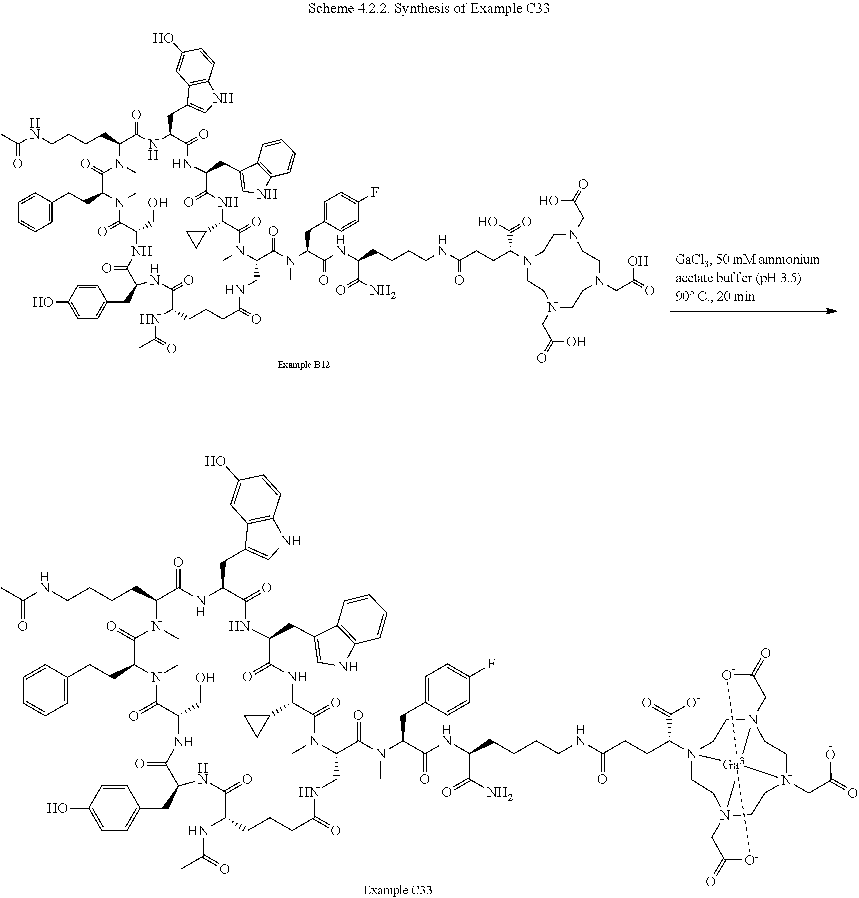

To Example B12 (30.0 mg, 1 eq., 13.98 μmol) in ammonium acetate buffer pH 3.5 (10.0 mL, 50 mM) was added GaCl₃ in ammonium acetate buffer pH 3.5 (5.6 μL, 5.0 M, 2.0 eq., 27.97 μmol). The reaction mixture was stirred at 90° C. for 20. The reaction mixture was lyophilized to afford the crude peptide. The product was isolated by preparative HPLC (Column: XSelect® Peptide CSH C18, OBD™ Prep Column, 130 Å, 5 μm, 30 mm×250 mm; 50 mL/min; Eluent A: H₂O+0.08% NH₄HCO₃ and eluent B: ACN). Pure fractions were combined and lyophilized to afford Example C33 (26.1 mg, 12 μmol, 84% yield) as a white solid. LCMS Method AP-1, t_{R}=3.99 min, [M+2H]²⁺=1106.5.

### 4.2.3 Synthesis of Example C34 and Example C35 (Example B13 Labeled with Ga) (B13 Disclosed as SEQ ID NO: 47, C34 Disclosed as SEQ ID NO: 73, and C35 Disclosed as SEQ ID NO: 74)

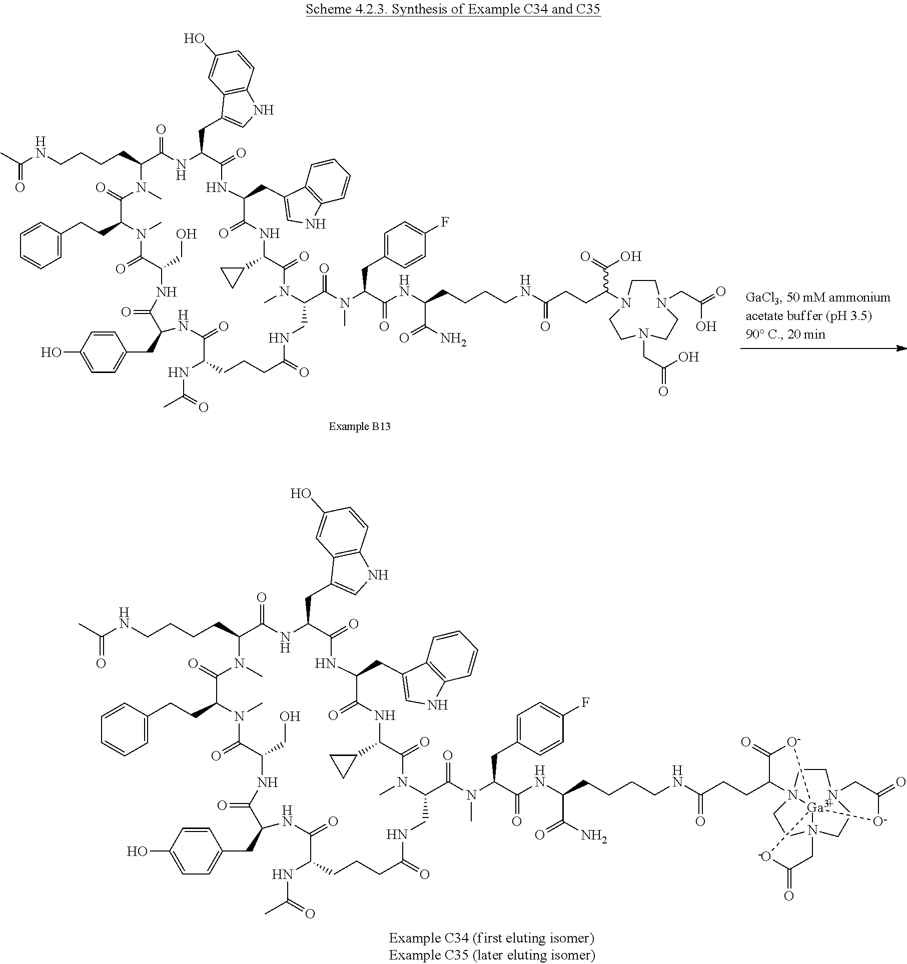

To Example B13 (40.0 mg, 1 eq., 19.57 μmol) in ammonium acetate buffer pH 3.5 (10.0 mL, 50 mM) was added GaCl₃ in ammonium acetate buffer pH 3.5 (7.8 μL, 5.0 M, 2.0 eq., 39.13 μmol). The reaction mixture was stirred at 90° C. for 20 min (clear solution when heated up). The reaction mixture was lyophilized to afford the crude peptide. The products were isolated by preparative HPLC (Column: XSelect® Peptide CSH C18, OBD™ Prep Column, 130 Å, 5 μm, 30 mm×250 mm; 50 mL/min; Eluent A: H₂O+0.08% NH₄HCO₃ and eluent B: ACN). Pure fractions containing the first eluting isomer were combined and lyophilized to afford Example C34 (3.4 mg, 1.5 μmol, 7.9% yield) as a white solid. LCMS Method AP-1, t_{R}=3.88 min, [M+H]⁺=2111.9. Pure fractions containing the later eluting isomer were combined and lyophilized to afford Example C35 (20.1 mg, 9.4 μmol, 48% yield) as a white solid. LCMS Method AP-1, t_{R}=4.23 min, [M+H]⁺=2111.9.

4.3 Labelling with Natural Lanthanum (La)

### 4.3.1 Synthesis of Example C30 (Example B1 Labeled with ¹³⁹La) (B1 Disclosed as SEQ ID NO: 36 and C30 Disclosed as SEQ ID NO: 69)

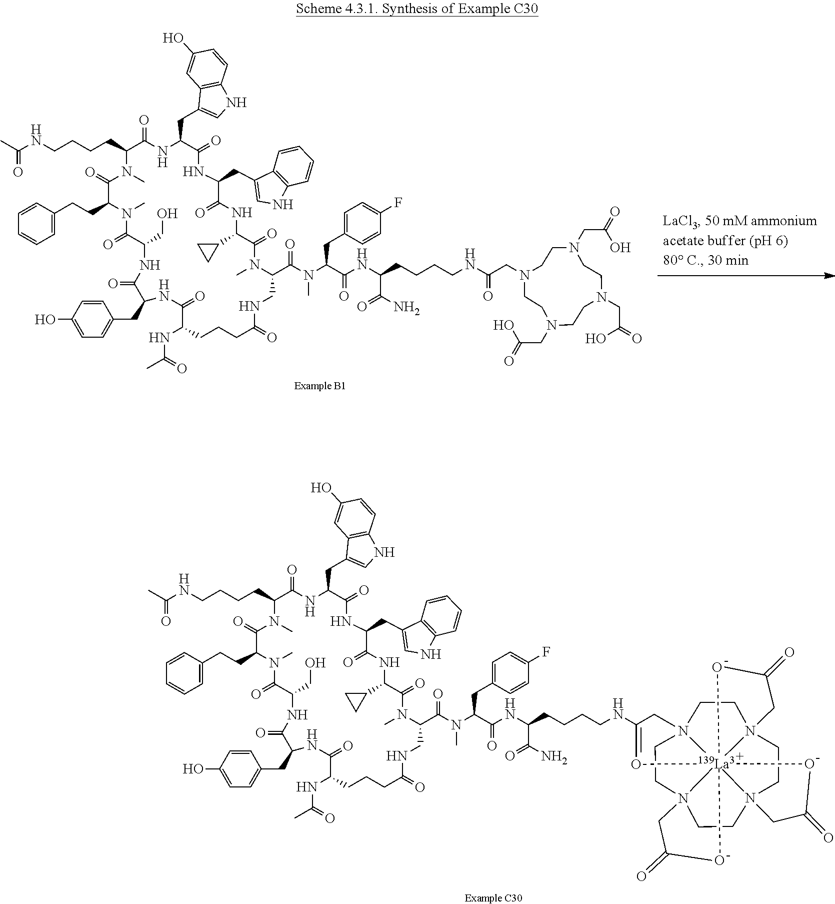

To Example B1 (27.0 mg, 1 eq., 10.67 μmol, TFA salt) in ammonium acetate buffer pH 6 (20.0 mL, 50 mM) was added 0.1 M LaCl₃ in ammonium acetate buffer pH 6 (213.5 μL, 0.1 M, 2.0 eq., 21.35 μmol). The reaction mixture was stirred at 80° C. for 30 min (clear solution when heated up). The reaction mixture was lyophilized to afford the crude peptide. The product was isolated by preparative HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example C30 (20.3 mg, 9.1 μmol, 85% yield) as a white solid. LCMS Method AP-1, t_{R}=4.19 min, [M+2H]²⁺=1105.5.

The following examples (for structures, see Table 4.8) were synthesized in analogy to Example C30:

Example C36 was synthesized from Example B4.

Example C37 was synthesized from Example B2.

TABLE 4
Examples synthesized in analogy to Example C30 (Scheme 4.3.1).

| Ex. No. | Structure/Description | LCMS |
|---|---|---|
| C36 (SEQ ID NO: 75) | 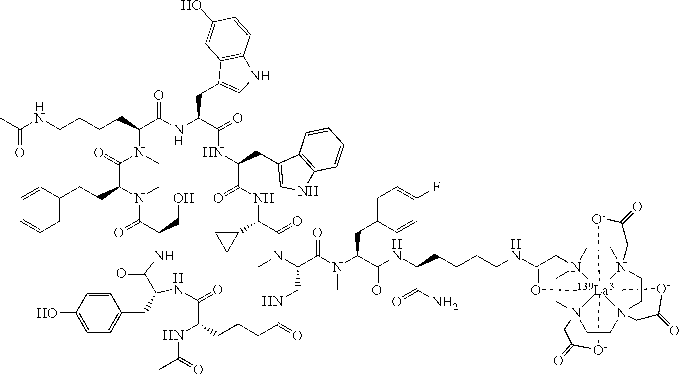 | Method AP-1 t_{R} = 4.34 min [M + H]⁺ = 2209.9 |
| | Example B4 labelled with ¹³⁹La | |
| C37 (SEQ ID NO: 76) | 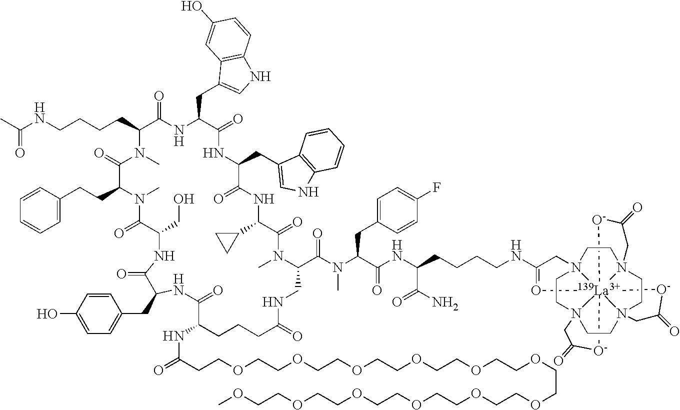 | Method AP-1 t_{R} = 4.56 min [M + H]⁺ = 2738.2 |
| | Example B2 labelled with ¹³⁹La | |

### 5 Dye Labeled Peptides (“D” Examples)

### 5.1 Synthesis of 2-((1E,3E)-5-((E)-1-(6-(((S)-5-((S)-2-((3S,6S,9S,12S,15S,18S,21S,24S,27S)-9-((1H-indol-3-yl)methyl)-27-acetamido-15-(4-acetamidobutyl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamido)-3-(4-fluorophenyl)propanamido)-6-amino-6-oxohexyl)amino)-6-oxohexyl)-3,3-dimethyl-5-sulfonatoindolin-2-ylidene)penta-1,3-dien-1-yl)-1-ethyl-3,3-dimethyl-3H-indol-1-ium-5-sulfonate (Example D1 (A40 disclosed as SEQ ID NO: 14 and D1 disclosed as SEQ ID NO: 77))

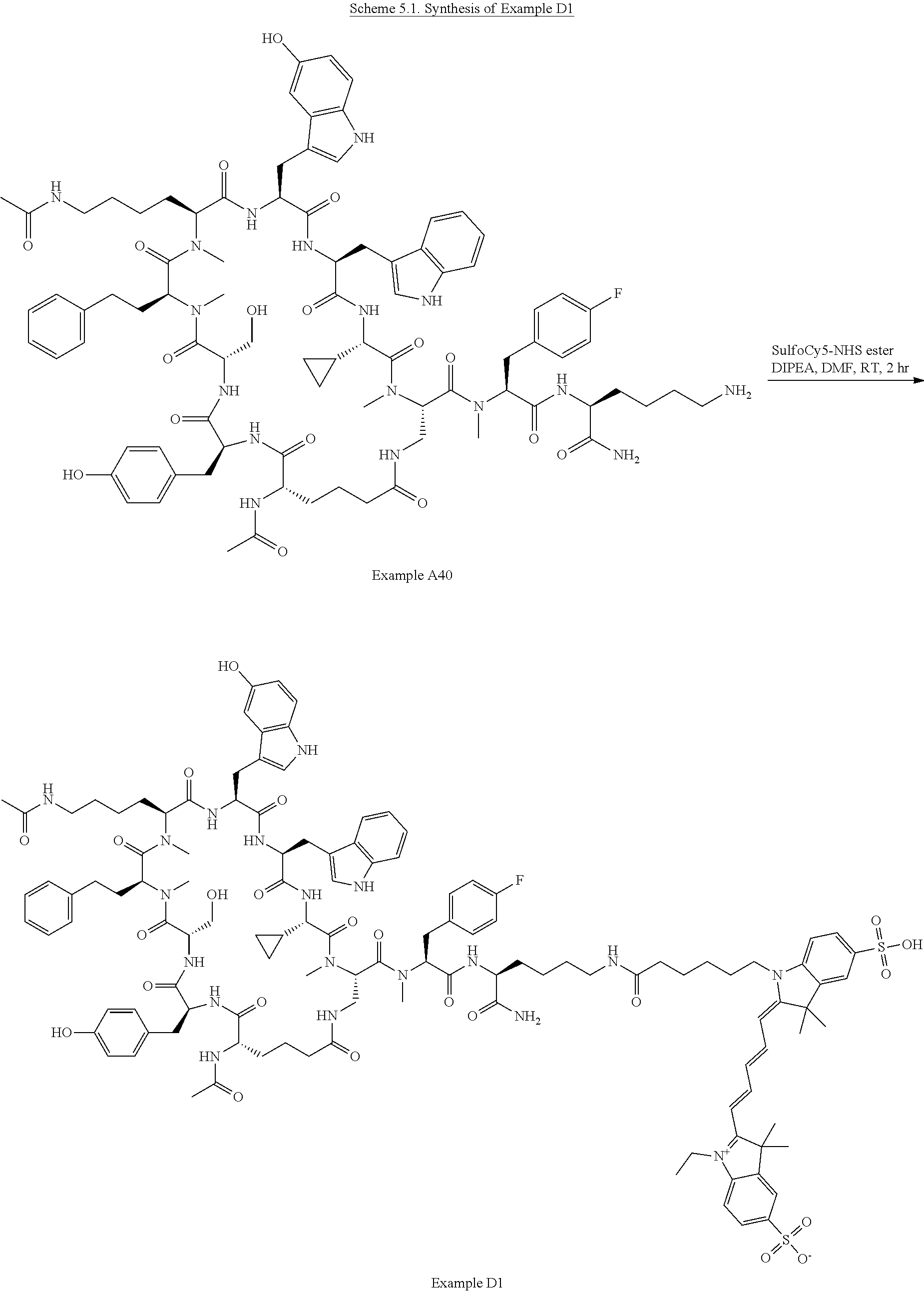

To a clear colorless solution of Example A40 (15 mg, 1 eq., 8.329 μmol, TFA salt) in DMF (0.3 mL) at RT were added SulfoCy5-NHS ester (8.16 mg, 1.3 eq., 10.83 μmol) and DIPEA (5.38 mg, 7.25 μL, 5 eq., 41.64 μmol). The resulting blue solution was stirred at RT for 2 hr. The reaction mixture was quenched with ACN/water (1/1) and the product was isolated by preparative RP HPLC (Column: XSelect® Peptide CSH C18, OBD™ Prep Column, 130 Å, 5 μm, 30×100 mm; Eluent A: H₂O+0.1% TFA and eluent B: ACN). Pure fractions were combined and lyophilized to afford Example D1 (12.9 mg, 5.5 μmol, 66% yield) as a blue solid. LCMS Method AP-1, t_{R}=5.09 min, [M+2H]²⁺=1163.0.

The following examples (for structures, see Table 5.1) were synthesized in analogy to Example D1:

Example D2 was synthesized from Example A45.

Example D7 was synthesized from Example A76.

TABLE 5.1
Examples synthesized in analogy to Example D1 (Scheme 5.1).

| Ex. No. | Structure/Example Name | LCMS |
|---|---|---|
| D2 (SEQ ID NO: 78) | 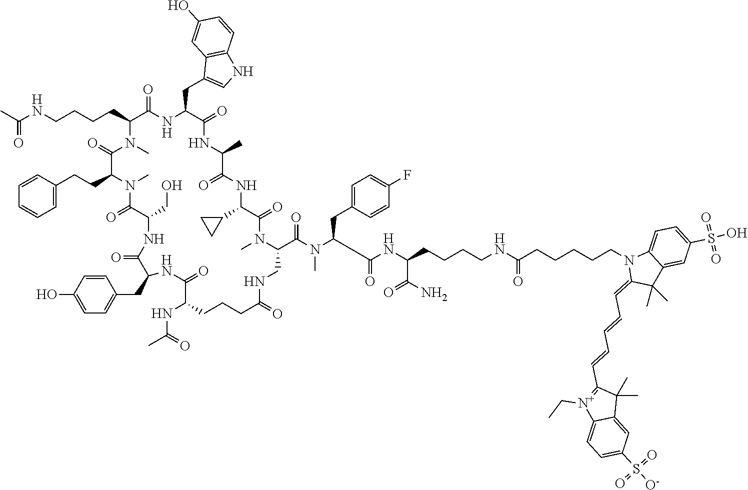 | Method AP-1 t_{R} = 4.53 min [M + H]⁺ = 2210.0 |
| | 2-((1E,3E)-5-((E)-1-(6-(((S)-5-((S)-2-((3S,6S,9S,12S,15S,18S,21S,24S,27S)-27-acetamido-15-(4-acetamidobutyl)-6-cyclopropyl- | |
| | 12-((5-hydroxy-1H-indol-3-yl)methyl)-24-(4-hydroxybenzyl)-21-(hydroxymethyl)-N,4,9,16,19-pentamethyl- | |
| | 5,8,11,14,17,20,23,26,31-nonaoxo-18-phenethyl-1,4,7,10,13,16,19,22,25-nonaazacyclohentriacontane-3-carboxamido)-3-(4- | |
| | fluorophenyl)propanamido)-6-amino-6-oxohexyl)amino)-6-oxohexyl)-3,3-dimethyl-5-sulfonatoindolin-2-ylidene)penta-1,3-dien- | |
| | 1-yl)-1-ethyl-3,3-dimethyl-3/-indol-1-ium-5-sulfonate | |
| D7 (SEQ ID NO: 83) | 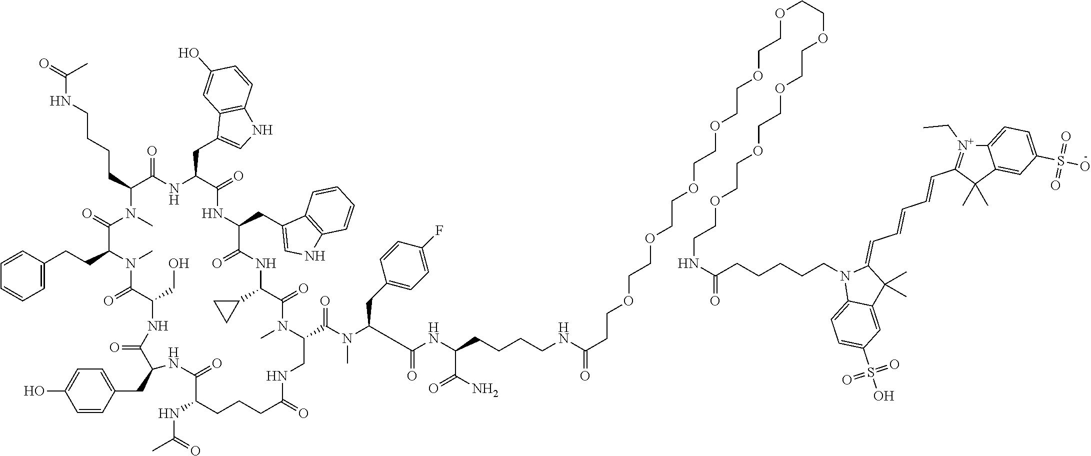 [SMILES: CC[N+]1=C(/C=C/C=C/C=C2/N(CCCCCC(=O)NCCOCCOCCOCCOCCOCCOCCOCCOCCOCCOCCC(=O)NCCCC[C@H](NC(=O)C[C@H](Cc3ccc(F)cc3)N(C)C(=O)[C@@H]3CNC(=O)CCC[C@H](NC(C)=O)C(=O)N[C@@H](Cc4ccc(O)cc4)C(=O)N[C@@H](CO)C(=O)N(C)[C@@H](CCc4ccccc4)C(=O)N(C)[C@@H](CCCCNC(C)=O)C(=O)N[C@@H](Cc4c[nH]c5ccc(O)cc45)C(=O)N[C@@H](Cc4c[nH]c5ccccc45)C(=O)N[C@@H](C4CC4)C(=O)N3C)C(N)=O)c3ccc(S(=O)(=O)O)cc3C2(C)C)C(C)(C)c2cc(S(=O)(=O)O)ccc21] | Method AP-1 t_{R} = 5.18 min [M + 2H]²⁺ = 1419.2 |
| | 2-((1E,3E)-5-((E)-1-((3S,6S)-1-((3S,6S,9S,12S,15S,18S,21S,24S,27S)-9-((1H-indol-3-yl)methyl)-27-acetamido-15-(4- | |
| | acetamidobutyl)-6-cyclopropyl-12-((5-hydroxy-1H-indol-3-yl)methyl)-24-(4-hydroxybenzyl)-21-(hydroxymethyl)-4,16,19- | |
| | trimethyl-5,8,11,14,17,20,23,26,31-nonaoxo-18-phenethyl-1,4,7,10,13,16,19,22,25-nonaazacyclohentriacontan-3-yl)-6- | |
| | carbamoyl-3-(4-fluorobenzyl)-2-methyl-1,4,12,46-tetraoxo-15,18,21,24,27,30,33,36,39,42-decaoxa-2,5,11,45- | |
| | tetraazahenpentacontan-51-yl)-3,3-dimethyl-5-sulfoindolin-2-ylidene)penta-1,3-dien-1-yl)-1-ethyl-3,3-dimethyl-3H-indol-1-ium- | |
| | 5-sulfonate | |

### 5.2 Synthesis of 2-((1E,3E)-5-((E)-1-((3S,6 S,43S)-1-((3S,6S,9S,12S,15S,18S,21S,24S,27S)-9-((1H-indol-3-yl)methyl)-27-acetamido-15-(4-acetamidobutyl)-12-((5-hydroxy-1H-indol-3-yl)methyl)-24-(4-hydroxybenzyl)-6-((S)-1-hydroxyethyl)-21-(hydroxymethyl)-4,16,19-trimethyl-5,8,11,14,17,20,23,26,30-nonaoxo-18-phenethyl-1,4,7,10,13,16,19,22,25-nonaazacyclotriacontan-3-yl)-43-carbamoyl-6-(4-(4-(carboxymethyl)piperazine-1-carboxamido)butyl)-3-(4-fluorobenzyl)-2-methyl-1,4,7,41,49-pentaoxo-11,14,17,20,23,26,29,32,35,38-decaoxa-2,5,8,42,48-pentaazatetrapentacontan-54-yl)-3,3-dimethyl-5-sulfoindolin-2-ylidene)penta-1,3-dien-1-yl)-1-ethyl-3,3-dimethyl-3H-indol-1-ium-5-sulfonate TFA salt (Example D3 (D3 disclosed as SEQ ID NO: 79))

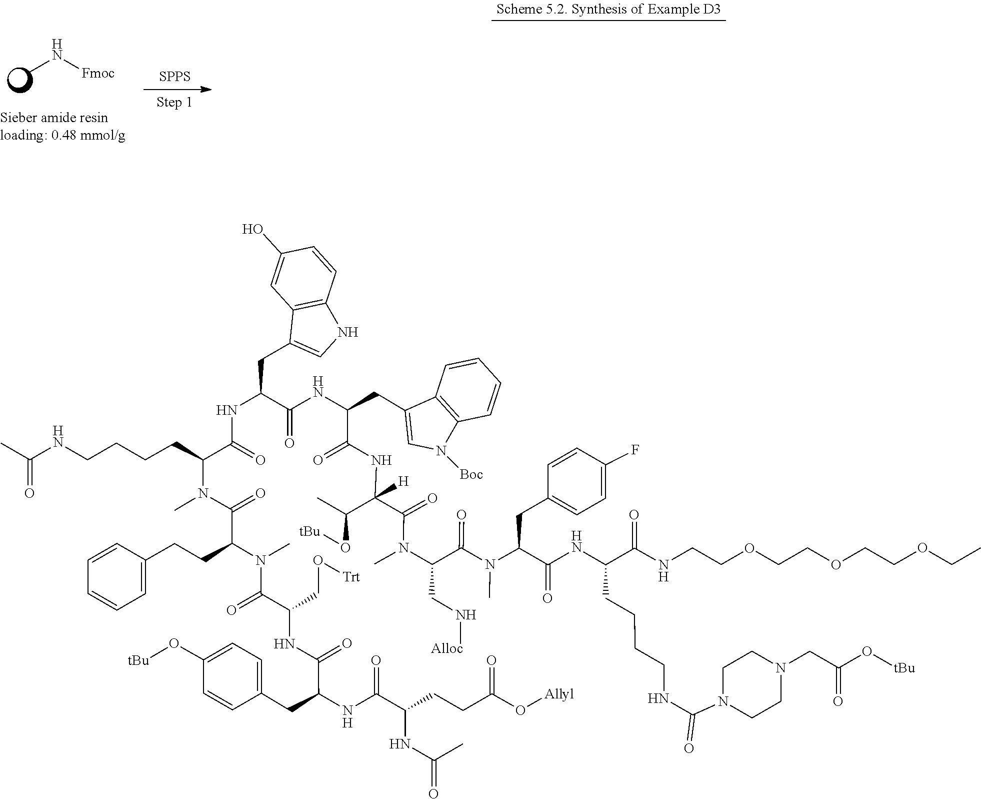

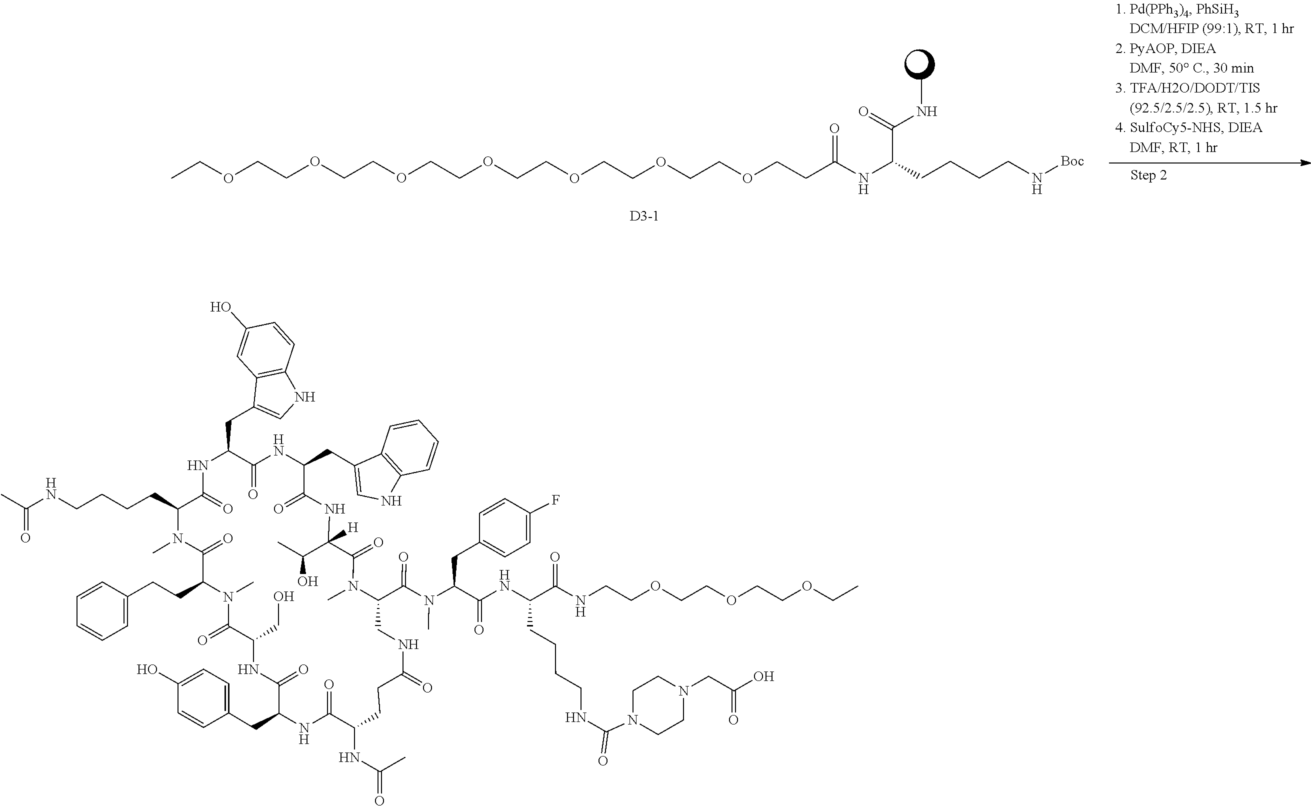

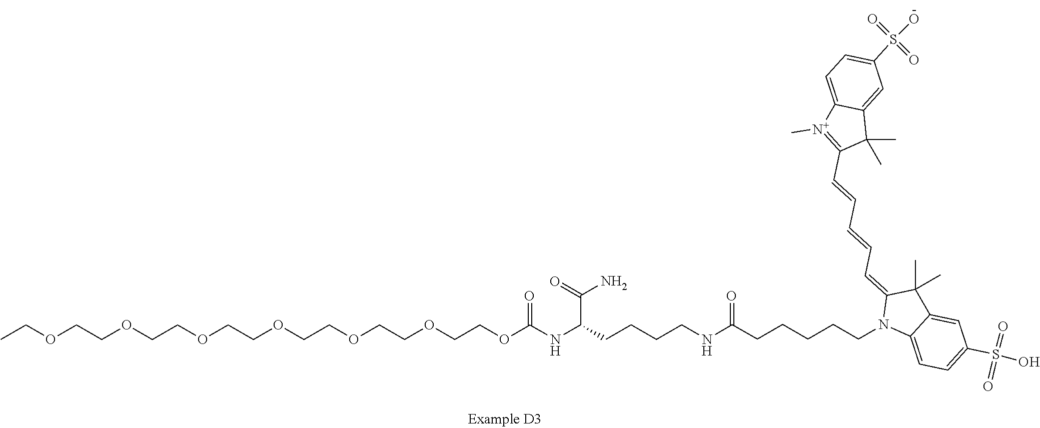 [SMILES: CCOCCOCCOCCOCCOCCOCCOC(=O)N[C@@H](CCCCNC(=O)CCCCCN1/C(=C/C=C/C=C/C2=[N+](C)c3ccc(S(=O)(=O)O)cc3C2(C)C)C(C)(C)c2cc(S(=O)(=O)O)ccc21)C(N)=O]

### Step 1. D3-1

The assembly of the linear peptide was performed on the CEM Liberty Blue Synthesizer and by manual coupling using commercial Sieber amide resin (loading 0.48 mmol/g, 521 mg, 0.25 mmol). The couplings were carried out using the following solutions: Fmoc-amino acid (0.21 M solution DMF, 5 mL, 4.2 eq.), DIC (1 M solution in DMF, 2 mL, 8 eq.), Oxyma Pure® (0.5 M solution in DMF, 2 mL, 4 eq.), addition by synthesizer. The Fmoc removal was performed using a solution of pyrrolidine (10% in DMF, 10 mL) or 53 mM Oxyma Pure® in pyrrolidine (5.3% in DMF, 19 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 2×3 min at 90° C.

Fmoc deprotection (after Synthesis cycle 13) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

### Method F (Manual Coupling)

The resin was treated with a 5% solution of Ac₂O in DCM (15 mL) and mixed at RT for 10 min.

After the assembly of the linear peptide the resin was washed with DMF, DCM, diethyl ether, and dried in vacuo. The amino acids and coupling methods are summarized in Table 5.2.

TABLE 5.2

| | Amino | | |
|---|---|---|---|
| Synthesis | acid | | SPPS |
| cycle | residue^{a} | Amino acid | Method |
| 1 | A¹⁰*** | Fmoc-L-Lys(Boc)-OH | A |
| 2 | A¹⁰** | Fmoc-PEG10-OH | A |
| 3 | A¹⁰* | Fmoc-L-K(COpipzaa)(OtBu)-OH | A |
| 4 | A¹⁰ | Fmoc-L-N-Me-Phe(4F)-OH | A |
| 5 | A⁹ | Fmoc-L-N-Me-Dap(Alloc)-OH | B |
| 6 | A⁸ | Fmoc-L-allo-Thr(tBu)-OH | C |
| 7 | A⁷ | Fmoc-L-Trp(Boc)-OH | D |
| 8 | A⁶ | Fmoc-L-Trp(5-OH)-OH | A |
| 9 | A⁵ | Fmoc-L-N-Me-Lys(Ac)-OH | A |
| 10 | A⁴ | Fmoc-L-N-Me-Homo-Phe-OH | B |
| 11 | A³ | Fmoc-L-Ser(Trt)-OH | E |
| 12 | A² | Fmoc-L-Tyr(tBu)-OH | D |
| 13 | A¹ | Fmoc-L-Glu(OAll)-OH | A + Fmoc |
| | | | Deprotection |
| 14 | A¹* | Ac₂O | F (Manual |
| | | | Coupling) |

^{a}Refers to the corresponding amino acid residue of cyclic peptide, P, as found, e.g., in Formulae (I), (Ia), (Ib), (Ic), (Id), or in the Table of Example III.

### Step 2. Example D3

Step 2-1: The resin from Step 1 (calculated with 250 μmol) was suspended with DCM/HFIP (99:1, 15 mL) and treated with phenylsilane (615 μL, 541 mg, 20 eq., 5.0 mmol) and tetrakis(triphenylphosphin) palladium (86.7 mg, 0.3 eq., 0.075 mmol). The suspension was shaken at RT for 1 hr. The solvent was drained, and then the resin was washed with DCM and DMF.

Step 2-2: The resin from Step 2-1 (calculated with 250 μmol) was suspended with DMF (10 mL). Then PyAOP (521 mg, 4.0 eq., 1.0 mmol) and DIPEA (340 μL, 8 eq., 2 mmol) were added. The reaction mixture was stirred at 50° C. for 30 min under microwave irradiation.

Step 2-3: To the resin from Step 2-2 was added TFA/H₂O/DODT/TIS (92.5/2.5/2.5/2.5) (9 mL) and the resin was shaken at RT for 1.5 hr. The resin was removed by filtration. The crude peptide was precipitated with diisopropyl ether/hexane (1/1) (80 mL) giving a precipitate. The suspension was centrifuged, and the solvent was decanted. The product was washed with another portion of diethyl ether. The suspension was centrifuged, and the solvent was decanted.

Step 2-4: The cyclic peptide from Step 2-3 was dissolved with DMF (25 mM, 10 mL). SulfoCy5-NHS ester (207 mg, 1.1 eq, 0.275 mmol) and DIPEA (128 μL, 3 eq, 0.75 mmol) were added. The resulting mixture was stirred for 1 hr at RT, quenched with AcOH, and concentrated in vacuo using EZ-II elite (Genevac, 40° C.).

The crude peptide was dried at RT under vacuo. The crude product was purified, and the product was isolated by preparative RP HPLC twice (Column: XSelect® Peptide CSH C18, OBD™ Prep Column, 130 Å, 5 μm, 19 mm×150 mm; Eluent A: H₂O+0.1% TFA and eluent B: ACN+0.1% TFA). Pure fractions were combined and lyophilized to afford Example D3 (9.38 mg, 2.9 μmol, 1.2% yield, TFA salt) as a white solid. LCMS Method AP-3, t_{R}=4.89 min, [M+3H]³⁺=1042.8.

### 5.3 Synthesis of 2-((1E,3E)-5-((E)-1-(6-(((S)-5-((S)-2-((3S,6S,9S,12S,15S,18S,21S,24S,27S)-9-((1H-indol-3-yl)methyl)-27-acetamido-15-(4-acetamidobutyl)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-6-amino-6-oxohexyl)amino)-6-oxohexyl)-3,3-dimethyl-5-sulfoindolin-2-ylidene)penta-1,3-dien-1-yl)-1-ethyl-3,3-dimethyl-3H-indol-1-ium-5-sulfonate (Example D4 (D4 disclosed as SEQ ID NO: 80))

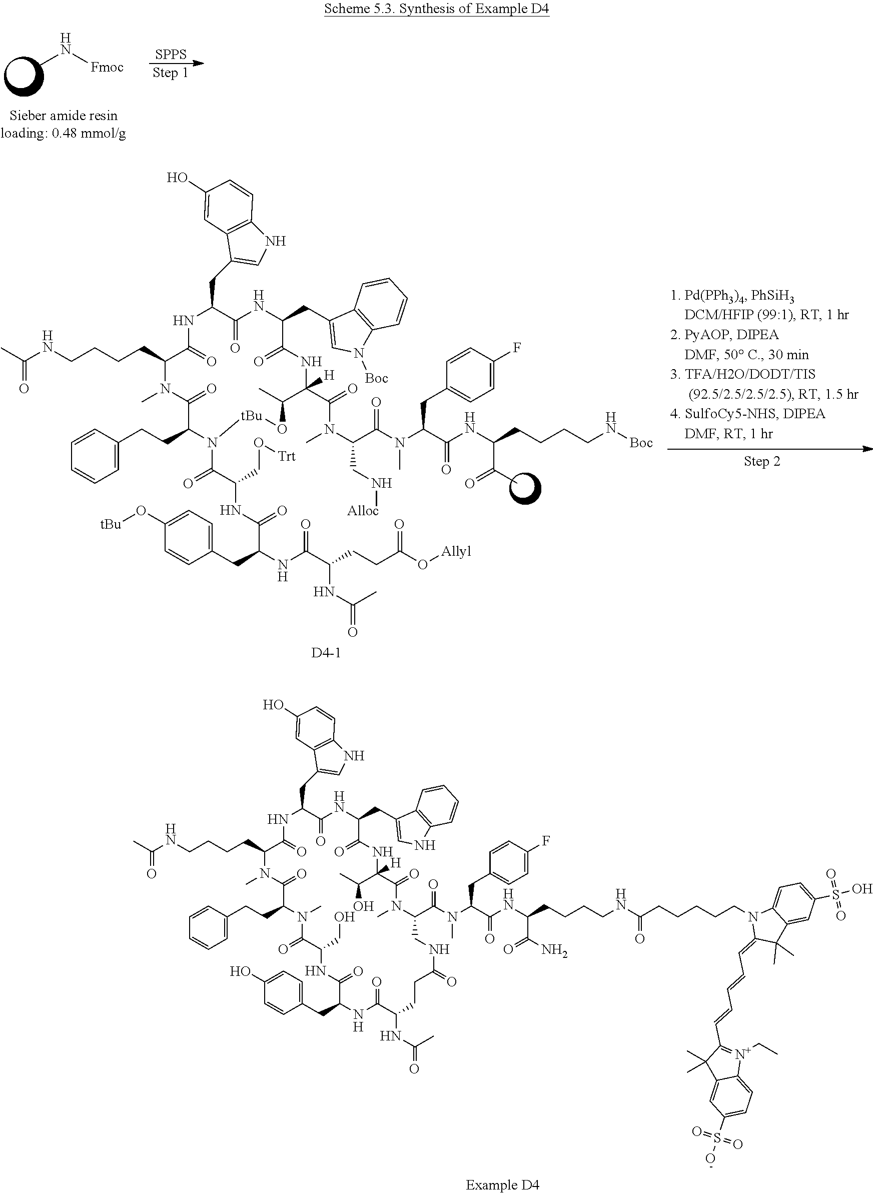

### Step 1. D4-1

The assembly of the linear peptide was performed on the CEM Liberty Blue Synthesizer and by manual coupling using commercial Sieber amide resin (loading 0.48 mmol/g, 521 mg, 0.25 mmol). The couplings were carried out using the following solutions: Fmoc-amino acid (0.21 M solution DMF, 5 mL, 4.2 eq.), DIC (1 M solution in DMF, 2 mL, 8 eq.), Oxyma Pure® (0.5 M solution in DMF, 2 mL, 4 eq.), addition by synthesizer. The Fmoc removal was performed using a solution of pyrrolidine (10% in DMF, 10 mL) or 53 mM Oxyma Pure® in pyrrolidine (5.3% in DMF, 19 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 2×3 min at 90° C.

Fmoc deprotection (after Synthesis cycle 11) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

### Method F (Manual Coupling)

The resin was treated with a 5% solution of Ac₂O in DCM (15 mL) and mixed at RT for 10 min.

After the assembly of the linear peptide the resin was washed with DMF, DCM, diethyl ether, and dried in vacuo. The amino acids and coupling methods are summarized in Table 5.3.

TABLE 5.3

| | Amino | | |
|---|---|---|---|
| Synthesis | acid | | |
| cycle | residue^{a} | Amino acid | SPPS Method |
| 1 | A¹⁰* | Fmoc-L-Lys(Boc)-OH | A |
| 2 | A¹⁰ | Fmoc-L-N-Me-Phe(4F)-OH | A |
| 3 | A⁹ | Fmoc-L-N-Me-Dap(Alloc)-OH | B |
| 4 | A⁸ | Fmoc-L-allo-Thr(tBu)-OH | C |
| 5 | A⁷ | Fmoc-L-Trp(Boc)-OH | D |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(Trt)-OH | E |
| 10 | A² | Fmoc-L-Tyr(tBu)-OH | D |
| 11 | A¹ | Fmoc-L-Glu(OAll)-OH | A + Fmoc |
| | | | Deprotection |
| 12 | A¹* | Ac₂O | F (Manual |
| | | | Coupling) |

^{a}Refers to the corresponding amino acid residue of cyclic peptide, P, as found, e.g., in Formulae (I), (Ia), (Ib), (Ic), (Id), or in the Table of Example III.

### Step 2. Example D4

The titled compound was synthesized in analogy to Step 2 of Example D3 to afford Example D4 (9.9 mg, 4.3 μmol, 1.7% yield) as a white solid. LCMS Method AP-3, t_{R}=5.43 min, [M+2H]²⁺=1158.7.

### 5.4 Synthesis of 2-((1E,3E)-5-((E)-1-(6-((3-(((3S,6S,9S,12S,15S,18S,21S,24S,27S)-9-((1H-indol-3-yl)methyl)-15-(4-acetamidobutyl)-3-(((S)-1-(((S)-1-amino-6-(4-(carboxymethyl)piperazine-1-carboxamido)-1-oxohexan-2-yl)amino)-3-(4-fluorophenyl)-1-oxopropan-2-yl)(methyl)carbamoyl)-12-((5-hydroxy-1H-indol-3-yl)methyl)-24-(4-hydroxybenzyl)-6-((S)-1-hydroxyethyl)-21-(hydroxymethyl)-4,16,19-trimethyl-5,8,11,14,17,20,23,26,30-nonaoxo-18-phenethyl-1,4,7,10,13,16,19,22,25-nonaazacyclotriacontan-27-yl)amino)-3-oxopropyl)amino)-6-oxohexyl)-3,3-dimethyl-5-sulfoindolin-2-ylidene)penta-1,3-dien-1-yl)-1-ethyl-3,3-dimethyl-3H-indol-1-ium-5-sulfonate TFA salt (Example D5 (D5 disclosed as SEQ ID NO: 81))

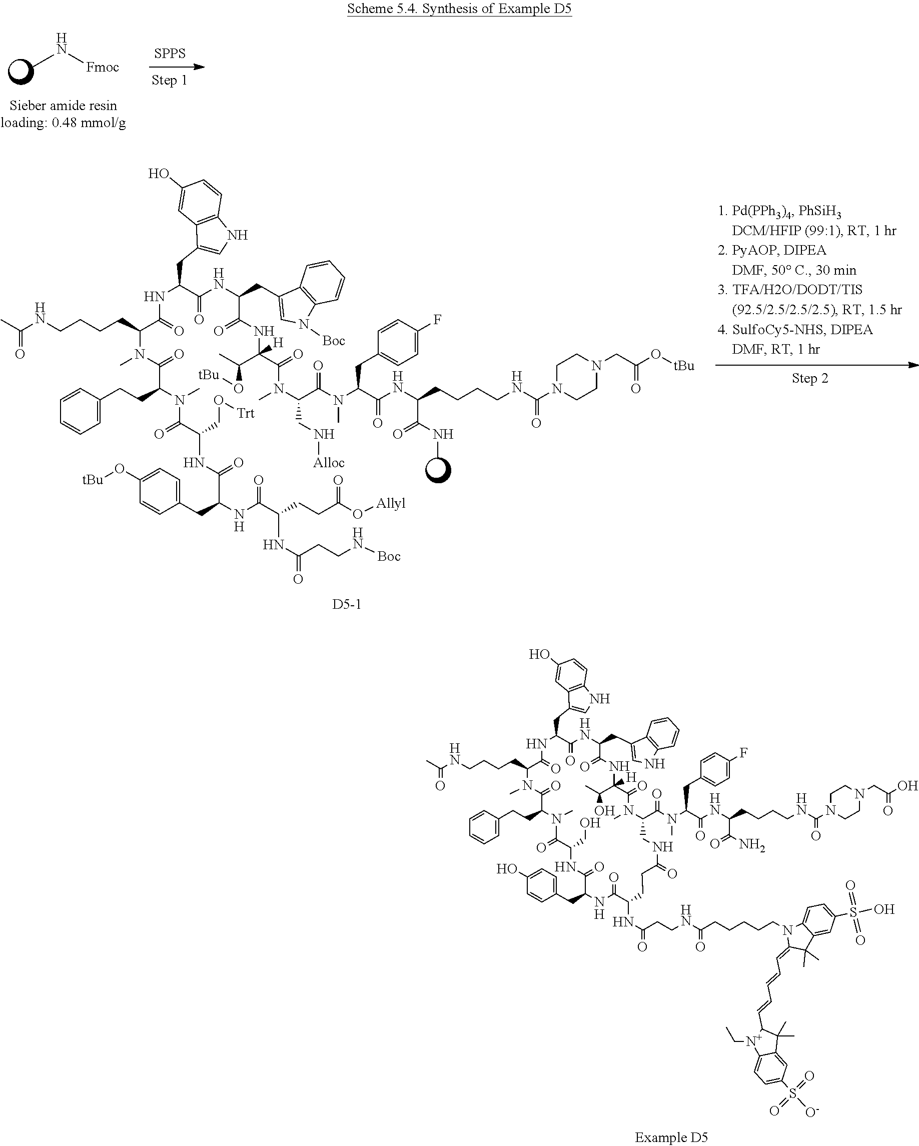

### Step 1. D5-1

The assembly of the linear peptide was performed on the CEM Liberty Blue Synthesizer using commercial Sieber amide resin (loading 0.48 mmol/g, 521 mg, 0.25 mmol). The couplings were carried out using the following solutions: Fmoc-amino acid (0.21 M solution DMF, 5 mL, 4.2 eq.), DIC (1 M solution in DMF, 2 mL, 8 eq.), Oxyma Pure® (0.5 M solution in DMF, 2 mL, 4 eq.), addition by synthesizer. The Fmoc removal was performed using a solution of pyrrolidine (10% in DMF, 10 mL) or 53 mM Oxyma Pure® in pyrrolidine (5.3% in DMF, 19 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 2×3 min at 90° C.

After the assembly of the linear peptide the resin was washed with DMF, DCM, diethyl ether, and dried in vacuo. The amino acids and coupling methods are summarized in Table 5.4.

TABLE 5.4

| Synthesis | Amino acid | | SPPS |
|---|---|---|---|
| cycle | residue^{a} | Amino acid | Method |
| 1 | A¹⁰* | Fmoc-L-K(COpipzaa)(OtBu)-OH | A |
| 2 | A¹⁰ | Fmoc-L-N-Me-Phe(4F)-OH | A |
| 3 | A⁹ | Fmoc-L-N-Me-Dap(Alloc)-OH | B |
| 4 | A⁸ | Fmoc-L-allo-Thr(tBu)-OH | C |
| 5 | A⁷ | Fmoc-L-Trp(Boc)-OH | D |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(Trt)-OH | E |
| 10 | A² | Fmoc-L-Tyr(tBu)-OH | D |
| 11 | A¹ | Fmoc-L-Glu(OAll)-OH | A |
| 12 | A¹* | Boc-L-Beta-Ala-OH | D |

^{a}Refers to the corresponding amino acid residue of cyclic peptide, P, as found, e.g., in Formulae (I), (Ia), (Ib), (Ic), (Id), or in the Table of Example III.

### Step 2. Example D5

The titled compound was synthesized in analogy to Step 2 of Example D3 to afford Example D5 (13.56 mg, 5.2 μmol, 2% yield, TFA salt) as a white solid. LCMS Method AP-3, t_{R}=4.68 min, [M+2H]²⁺=1258.2.

### 5.5 Synthesis of Example D6 (D6-1 Disclosed as SEQ ID NO: 115, D6-2 Disclosed as SEQ ID NO: 116, and D6 Disclosed as SEQ ID NO: 82)

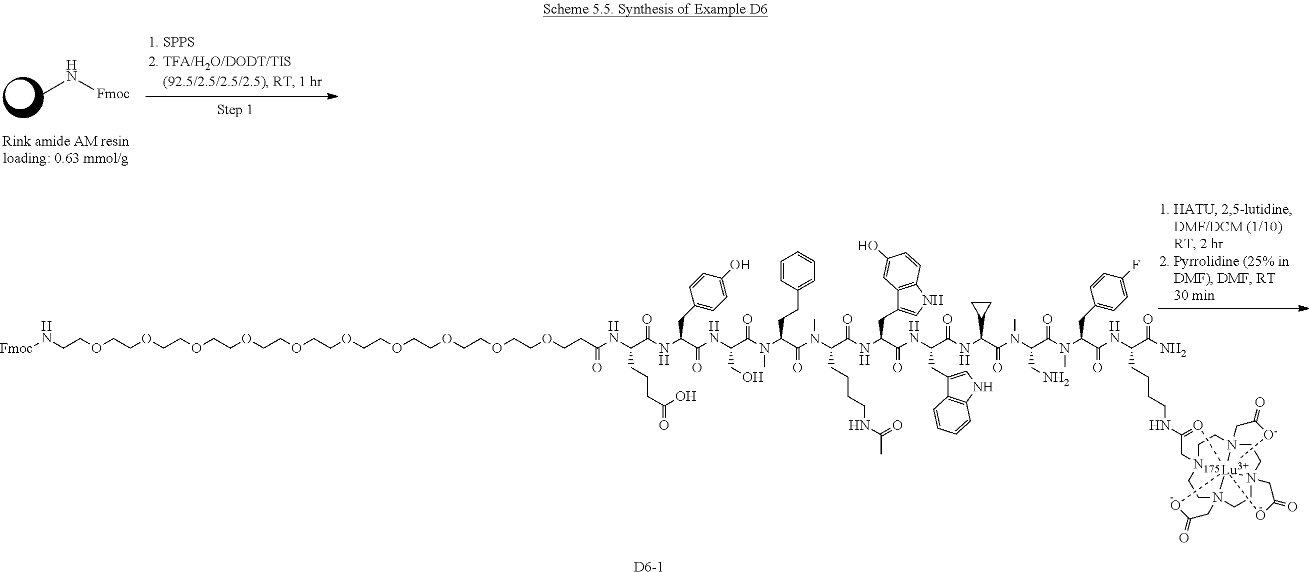

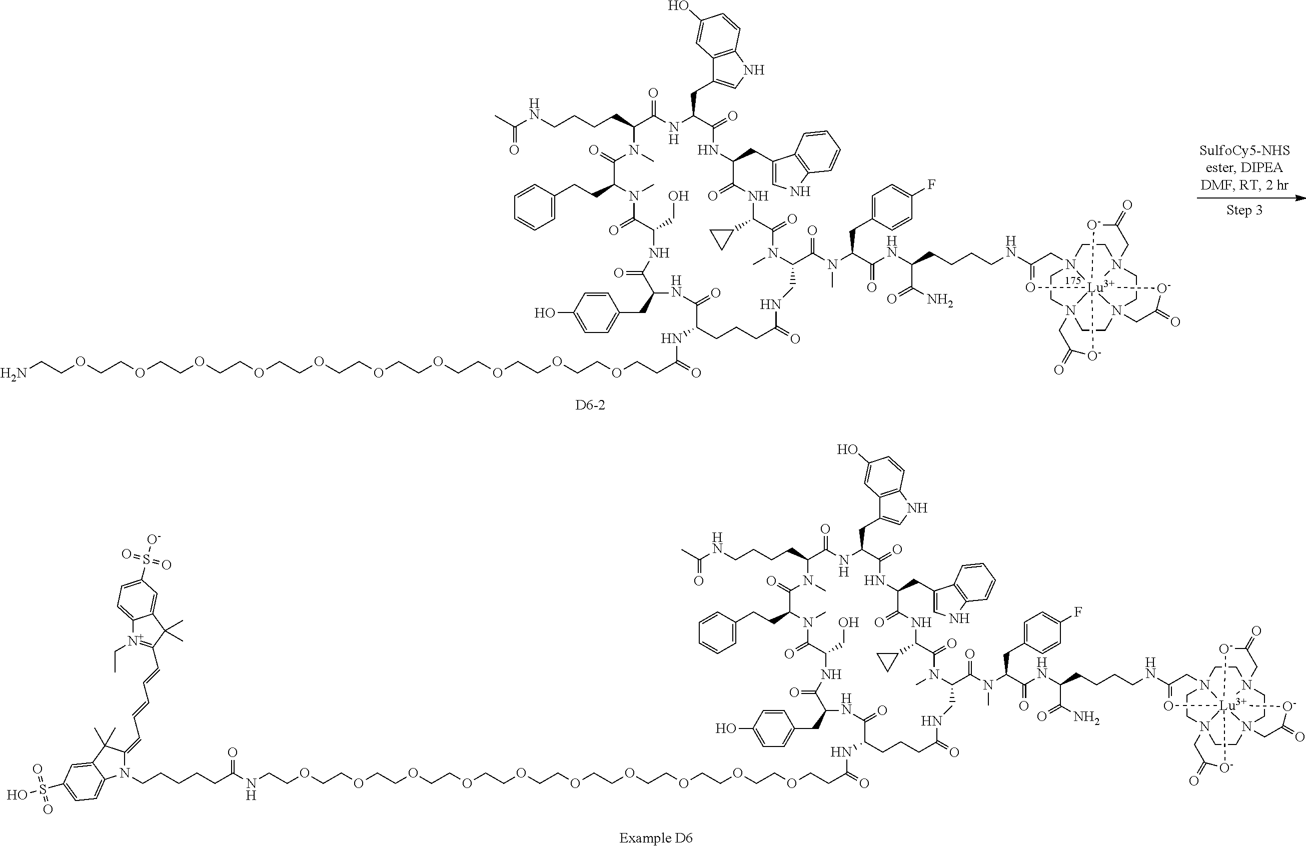

### Step 1. D6-1

Step 1-1: The assembly of the linear peptide was done on the CEM Liberty Prime Synthesizer and by manual coupling using commercial Rink amide AM resin R2 (loading 0.63 mmol/g, 317 mg, 0.200 mmol). The couplings were carried out using the following solutions: Fmoc-amino acid (0.5 M solution DMF, 2 mL, 5 eq.), DIC (2 M solution in DMF, 1 mL, 10 eq.), Oxyma Pure® (0.25 M solution in DMF, 4 mL, 5 eq.), addition by synthesizer. Fmoc removal was performed using a solution of pyrrolidine (25% in DMF, 1.5 mL), addition by synthesizer.

The amino acids were coupled with a set of different methods.
Method A Fmoc removal: 1×1 min at 110° C./Coupling: 1×4 min at 105° C.
Method B Fmoc removal: 2×10 min at RT/Coupling: 2×4 min at 105° C.
Method C Fmoc removal: 2×10 min at RT/Coupling: 2×8 min at 105° C.

Fmoc deprotection (after Synthesis cycle 11) was performed using 25% pyrrolidine in DMF (1.5 mL), addition by synthesizer, 1×1 min at 110° C.

### Method D (Manual Coupling)

The resin was treated with a pre-activated solution of Fmoc-PEG10-OH (180 mg, 1.2 eq., 0.24 mmol), HATU (114 mg, 1.5 eq., 0.30 mmol) and DIPEA (129 mg, 174 μL, 5 eq., 1.0 mmol) in DMF (3 mL) and mixed at RT for 2 hr.

After the assembly of the linear peptide the resin was washed with DMF (5×) and DCM (5×). The amino acids and coupling methods are summarized in Table 5.5.

TABLE 5.5

| Synthesis | Amino acid | | |
|---|---|---|---|
| cycle | residue^{a} | Amino acid | Method |
| 1 | A¹⁰ | BB5 | C |
| 2 | A⁹ | Fmoc-L-N-Me-Phe(4F)-OH | A |
| 3 | A⁸ | Fmoc-L-N-Me-Dap(Boc)-OH | B |
| 4 | A⁷ | Fmoc-L-CyclopropylGly-OH | B |
| 5 | A⁶ | Fmoc-L-Trp(Boc)-OH | B |
| 6 | A⁵ | Fmoc-L-Trp(5-OH)-OH | A |
| 7 | A⁴ | Fmoc-L-N-Me-Lys(Ac)-OH | A |
| 8 | A³ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | B |
| 11 | A¹ | Fmoc-L-Homo-Glu(OtBu)-OH | A + Fmoc |
| | | | deprotection |
| 12 | A¹* | Fmoc-PEG10-OH | D (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (10 mL) and the resin was shaken at RT for 3 hr. The crude peptide was precipitated with cold heptane/MTBE (90 mL). The suspension was centrifuged, and the solvent was decanted. The product was washed with another portion of cold heptane/MTBE (80 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford D6-1 (40 mg, 12.0 μmol, 6.2% yield, TFA salt) as a white solid. LCMS Method AP-1, t_{R}=4.82 min, [M+2H]²⁺=1478.2.

### Step 2. D6-2

Step 2-1: D6-1 (40 mg, 1 eq., 13.03 μmol TFA salt) was dissolved in DMF/DCM (1/10) (33 mL). Then HATU (5.45 mg, 1.1 eq., 14.34 μmol), followed by 2,6-lutidine (27.9 mg, 30.2 μL, 20 eq., 260.7 μmol) were added. The reaction mixture was stirred at RT for 2 hr and then concentrated at 30° C. in vacuo to remove the DCM. The residue was precipitated with cold MTBE (40 mL). The suspension was centrifuged, and the solvent was decanted. The product was washed with another portion of cold MTBE (40 mL). The suspension was centrifuged, and the solvent was decanted to afford the crude peptide.

Step 2-2: The crude peptide from Step 2-1 was dissolved in DMF (1 mL) and pyrrolidine (25% in DMF) (74.2 mg, 85.6 μL, 25 eq., 260.7 μmol) was added at RT. The reaction mixture was stirred at RT for 30 min, then quenched with ACN/water (1/1). The product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford D6-2 (13.9 mg, 4.6 μmol, 35% yield, TFA salt) as a white solid. LCMS Method AP-1, t_{R}=3.84 min, [M+H]=1358.1.

### Step 3. Example D6

To a clear colorless solution of D6-2 (13.9 mg, 1 eq., 4.914 μmol, TFA salt) in DMF (0.5 mL) at RT were added SulfoCy5-NHS ester (5.56 mg, 1.5 eq., 7.37 μmol) and DIPEA (3.2 mg, 4.3 μL, 5 eq., 24.57 μmol). The resulting blue solution was stirred at RT for 2 hr. The reaction mixture was quenched with ACN/water (1/1) and the product was isolated by preparative RP HPLC (Column: XSelect® Peptide CSH C18, OBD™ Prep Column, 130 Å, 5 μm, 30 mm×100 mm; Eluent A: H₂O+0.1% TFA and eluent B: ACN). Pure fractions were combined and lyophilized to afford Example D6 (4.9 mg, 1.4 μmol, 29% yield) as a blue solid. LCMS Method AP-1, t_{R}=4.68 min, [M+2H]²⁺=1677.2.

### 6 Biotinylated Peptides (“E” Examples)

### 6.1 Synthesis of (3S,6S,9S,12S,15S,18S,21S,24S,27S)-9-((1H-indol-3-yl)methyl)-27-acetamido-15-(4-acetamidobutyl)-N-((2S,5S)-5-carbamoyl-1-(4-fluorophenyl)-3,11,45-trioxo-49-((3aS,4S,6aR)-2-oxohexahydro-1H-thieno[3,4-d]imidazol-4-yl)-14,17,20,23,26,29,32,35,38,41-decaoxa-4,10,44-triazanonatetracontan-2-yl)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamide (Example E1 (E1-2 disclosed as SEQ ID NO: 117 and E1 disclosed as SEQ ID NO: 84))

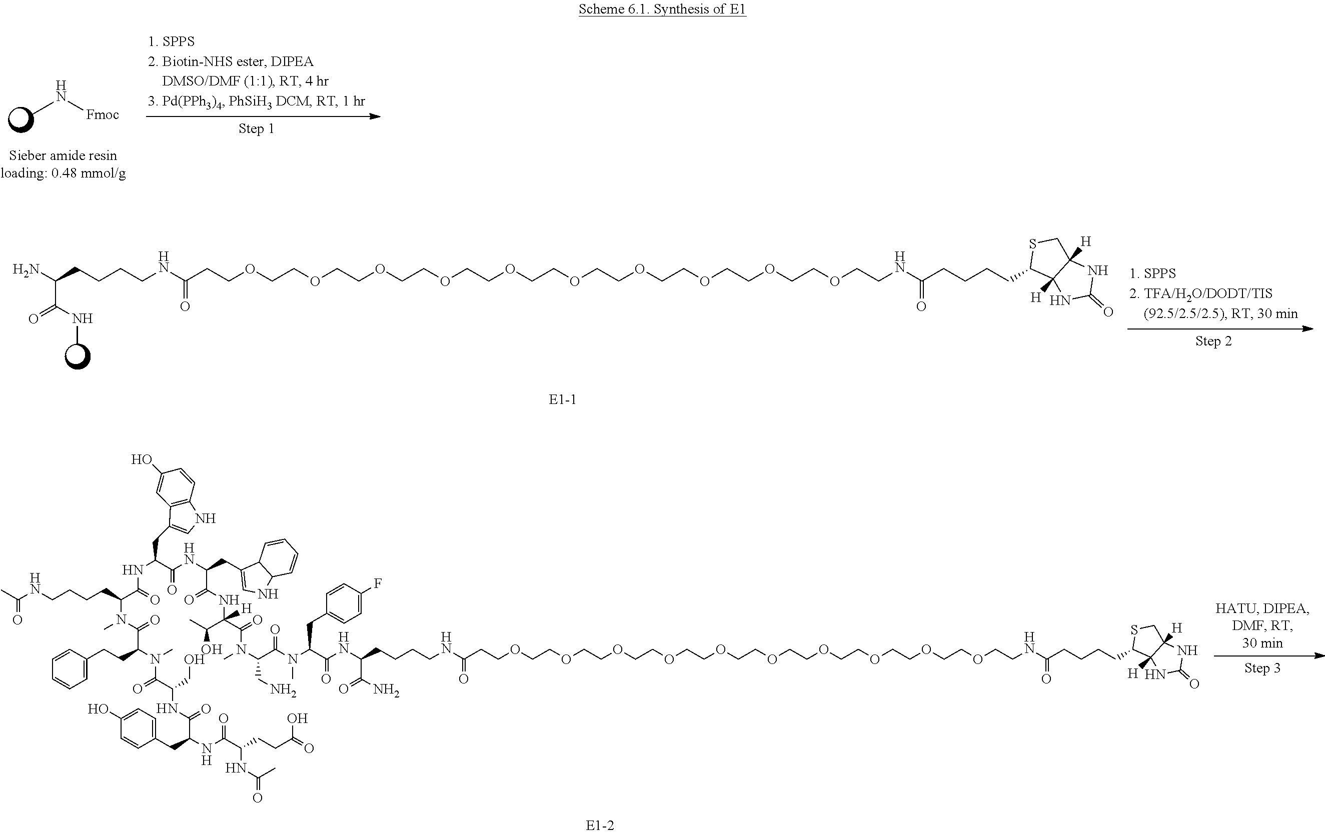

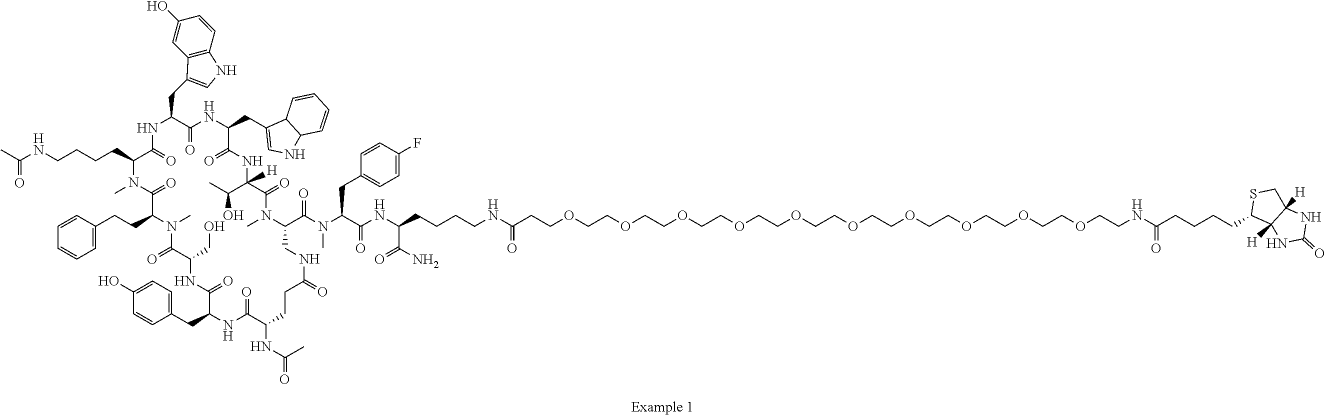 [SMILES: CC(=O)NCCCC[C@H]1C(=O)N[C@@H](Cc2c[nH]c3ccc(O)cc23)C(=O)N[C@@H](CC2=CNC3C=CC=CC23)C(=O)N[C@@H]([C@H](C)O)C(=O)N(C)[C@H](C(=O)N(C)[C@@H](Cc2ccc(F)cc2)C(=O)N[C@@H](CCCCNC(=O)CCOCCOCCOCCOCCOCCOCCOCCOCCOCCOCCNC(=O)CCCC[C@@H]2SC[C@@H]3NC(=O)N[C@@H]32)C(N)=O)CNC(=O)CC[C@H](NC(C)=O)C(=O)N[C@@H](Cc2ccc(O)cc2)C(=O)N[C@@H](CO)C(=O)N(C)[C@@H](CCc2ccccc2)C(=O)N1C]

### Step 1. E1-1

Step 1-1: The assembly of the linear peptide was done on the CEM Liberty Blue Synthesizer using commercial Sieber Amide resin R3 (loading 0.48 mmol/g, 260 mg, 0.125 mmol). The amino acids were coupled with the following method.

### Method A

Fmoc-amino acid (0.21 M solution in DMF, 2.5 mL, 4.2 eq.), DIC (1 M solution in DMF, 1 mL, 8 eq.), Oxyma Pure® (0.5 M solution in DMF, 1 mL, 4 eq.), addition by synthesizer, 1×3 min at 90° C. The Fmoc removal was performed using a solution of pyrrolidine (10% in DMF, 5 mL) or 53 mM Oxyma Pure® in pyrrolidine (5.3% in DMF, 9.5 mL), addition by synthesizer, 1×1 min at 90° C.

After the assembly of the linear peptide the resin was washed with DMF. The amino acids and coupling methods are summarized in Table 6.1.

TABLE 6.1

| Synthesis | Amino acid | | |
|---|---|---|---|
| cycle | residue^{a} | Amino acid | SPPS Method |
| 1 | A¹⁰* | Alloc-L-Lys(Fmoc)-OH | A |
| 2 | A¹⁰** | Fmoc-PEG10-OH | A |

^{a}Refers to the corresponding amino acid residue of cyclic peptide, P, as found, e.g., in Formulae (I), (Ia), (Ib), (Ic), (Id), or in the Table of Example III.

Step 1-2: The resin from Step 1-1 (calculated with 125 μmol) was suspended with DMSO/DMF (1:1, 7 mL) and treated with Biotin-NHS ester (171 mg, 4 eq., 0.5 mmol) and DIPEA (106 μL, 5 eq., 0.625 mmol). The suspension was shaken at RT for 4 hr. The solvent was drained, and then the resin was washed with DMF and DCM.

Step 1-3: The resin from Step 1-2 (calculated with 125 μmol) was suspended with DCM (6 mL) and treated with phenylsilane (154 μL, 135 mg, 10 eq., 1.25 mmol) and tetrakis(triphenylphosphin) palladium (28.9 mg, 0.2 eq., 0.025 mmol). The suspension was shaken at RT for 1 hr. The solvent was drained, and then the resin was washed with DCM and DMF.

### Step 2. E1-2

Step 2-1: The assembly of the linear peptide was performed on the CEM Liberty Blue Synthesizer and by manual coupling using the resin from Step 1 (calculated with 125 μmol). The couplings were carried out using the following solutions: Fmoc-amino acid (0.21 M solution DMF, 2.5 mL, 4.2 eq.), DIC (1 M solution in DMF, 1 mL, 8 eq.), Oxyma Pure® (0.5 M solution in DMF, 1 mL, 4 eq.), addition by synthesizer. The Fmoc removal was performed using a solution of pyrrolidine (10% in DMF, 5 mL) or 53 mM Oxyma Pure® in pyrrolidine (5.3% in DMF, 9.5 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 1×10 min at 90° C.
Method C Fmoc removal: 1×1 min at 90° C./Coupling: 2×3 min at 90° C.
Method D Fmoc removal: 2×1 min at RT/Coupling: 2×30 min at 75° C.
Method E Fmoc removal: 2×1 min at RT/Coupling: 1×3 min at 90° C.
Method F Fmoc removal: 2×1 min at RT/Coupling: 2×3 min at 90° C.

Fmoc deprotection (after Synthesis cycle 10) was performed using a solution of pyrrolidine (10% in DMF, 15 mL) or 53 mM Oxyma Pure® in pyrrolidine (5.3% in DMF, 28.5 mL), addition by synthesizer, 2×1 min at RT.

### Method G (Manual Coupling)

The resin was treated with a 5% solution of Ac₂O in DCM (6 mL) and mixed at RT for 10 min.

After the assembly of the linear peptide the resin was washed with DMF, DCM, diethyl ether, and dried in vacuo. The amino acids and coupling methods are summarized in Table 6.2.

TABLE 6.2

| Synthesis | Amino acid | | |
|---|---|---|---|
| cycle | residue^{a} | Amino acid | SPPS Method |
| 1 | A¹⁰ | Fmoc-L-N-Me-Phe(4F)-OH | B |
| 2 | A⁹ | Fmoc-L-N-Me-Dap(Boc)-OH | C |
| 3 | A⁸ | Fmoc-L-allo-Thr(tBu)-OH | D |
| 4 | A⁷ | Fmoc-L-Trp(Boc)-OH | E |
| 5 | A⁶ | Fmoc-L-Trp(5-OH)-OH | A |
| 6 | A⁵ | Fmoc-L-N-Me-Lys(Ac)-OH | A |
| 7 | A⁴ | Fmoc-L-N-Me-Homo-Phe-OH | C |
| 8 | A³ | Fmoc-L-Ser(Trt)-OH | F |
| 9 | A² | Fmoc-L-Tyr(tBu)-OH | E |
| 10 | A¹ | Fmoc-L-Glu(tBu)-OH | A + Fmoc |
| | | | deprotection |
| 11 | A¹* | Ac₂O | G (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

Step 2-2: To the resin from Step 2-1 was added TFA/H₂O/DODT/TIS (92.5/2.5/2.5/2.5) (6 mL) and the resin was shaken at RT for 30 min. The resin was removed by filtration. The crude peptide was precipitated with diisopropyl ether/hexane (1/1) (40 mL). The suspension was centrifuged, and the solvent was decanted. The product was washed with another portion of diethyl ether. The suspension was centrifuged, and the solvent was decanted. The crude peptide was dried at RT in vacuo to afford E1-2.

### Step 3. Example E1

E1-2 (125 μmol, TFA salt) was dissolved in DMF (5 mM, 4 mL). Then HATU (57.0 mg, 1.2 eq., 150 μmol), followed by DIPEA (63.8 μL, 3 eq., 375 μmol) were added. The reaction mixture was stirred at RT for 30 min. The reaction mixture was quenched with AcOH.

The reaction mixture was directly purified by preparative RP HPLC (Column: XSelect® Peptide CSH C18, OBD™ Prep Column, 130 Å, 5 μm, 19 mm×150 mm; Eluent A: H₂O+0.1% TFA and eluent B: ACN+0.1% TFA). Pure fractions were combined and lyophilized to afford Example E1 (16.17 mg, 6.4 μmol, 5.1% yield) as a white solid. LCMS Method AP-3, t_{R}=5.20 min, [M+2H]²⁺=1208.3.
6.2 Synthesis of 2-(4-(((11S,48S)-48-((S)-2-((3S,6S,9S,12S,15S,18S,21S,24S,27S)-9-((1H-indol-3-yl)methyl)-27-acetamido-15-(4-acetamidobutyl)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-11-carbamoyl-5,13,47-trioxo-1-((3aS,4S,6aR)-2-oxohexahydro-1H-thieno[3,4-d]imidazol-4-yl)-16,19,22,25,28,31,34,37,40,43-decaoxa-6,12,46-triazadopentacontan-52-yl) carbamoyl) piperazin-1-yl) acetic acid TFA salt (Example E2 (E2-2 disclosed as SEQ ID NO: 118 and E2 disclosed as SEQ ID NO: 85))

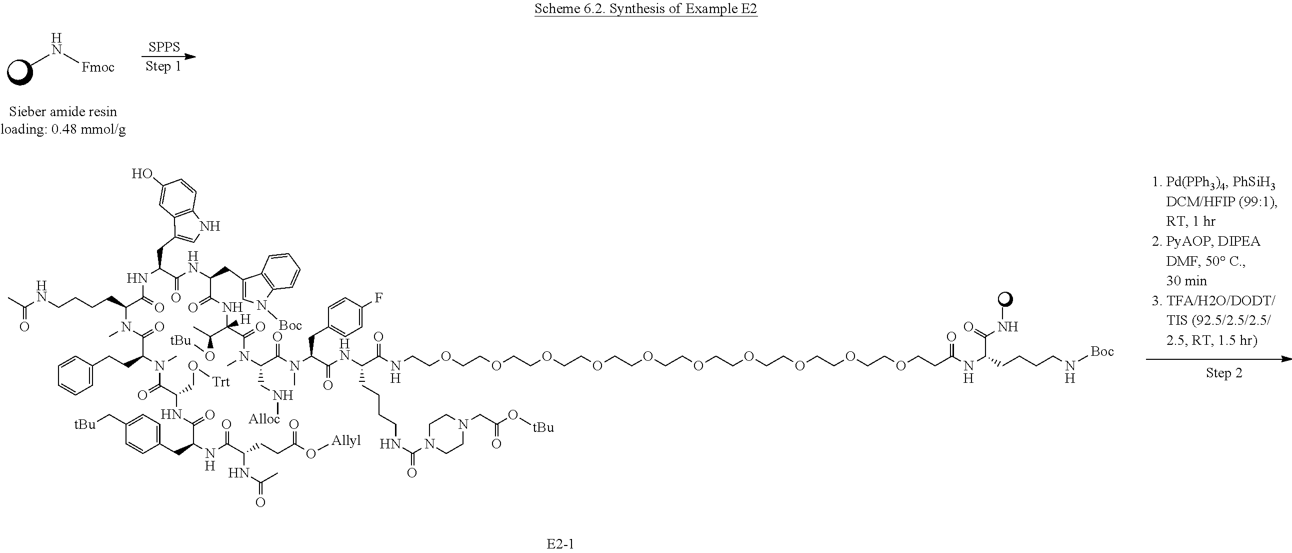

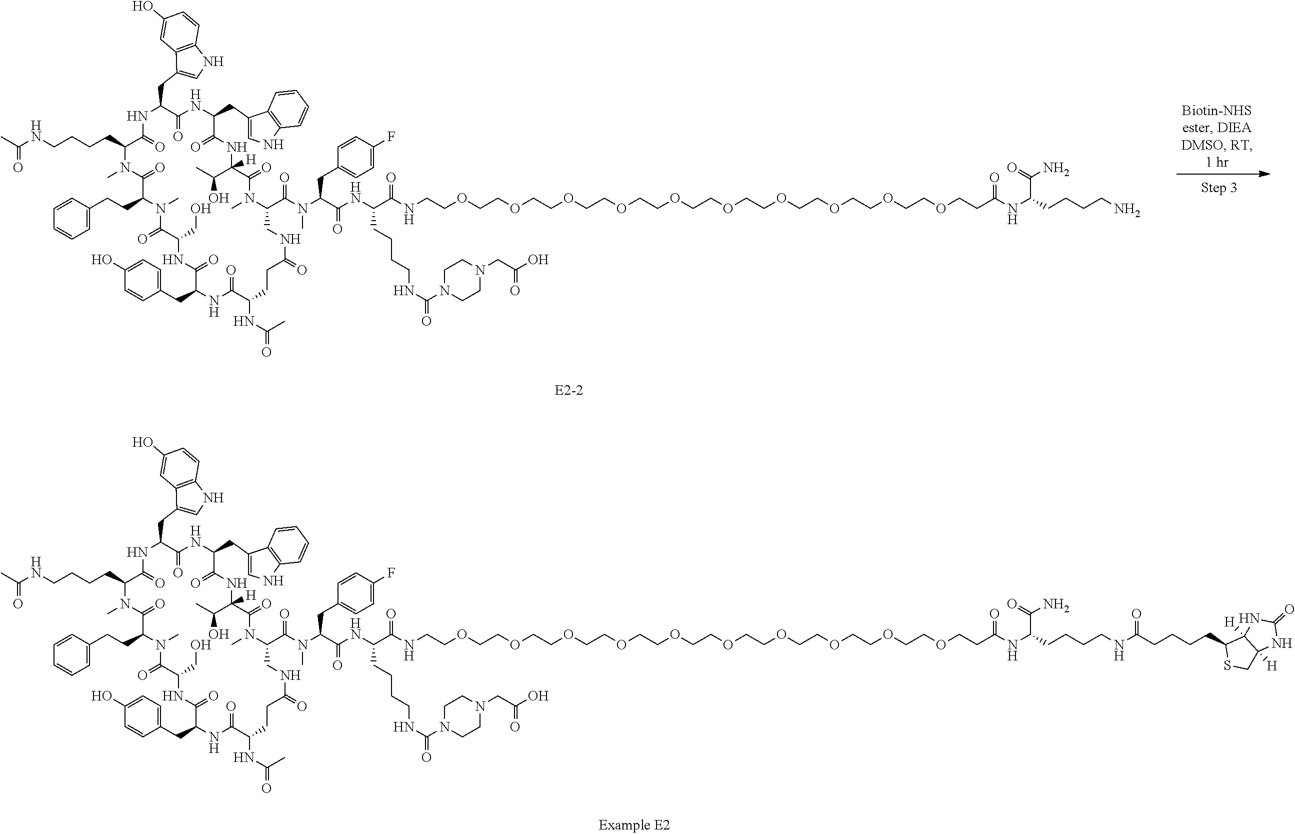

### Step 1. E2-1

The assembly of the linear peptide was performed on the CEM Liberty Blue Synthesizer and by manual coupling using commercial Sieber amide resin (loading 0.48 mmol/g, 521 mg, 0.25 mmol). The couplings were carried out using the following solutions: Fmoc-amino acid (0.21 M solution DMF, 5 mL, 4.2 eq.), DIC (1 M solution in DMF, 2 mL, 8 eq.), Oxyma Pure® (0.5 M solution in DMF, 2 mL, 4 eq.), addition by synthesizer. The Fmoc removal was performed using a solution of pyrrolidine (10% in DMF, 10 mL) or 53 mM Oxyma Pure® in pyrrolidine (5.3% in DMF, 19 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 2×3 min at 90° C.

Fmoc deprotection (after Synthesis cycle 13) was performed using a solution of pyrrolidine (10% in DMF, 10 mL) or 53 mM Oxyma Pure® in pyrrolidine (5.3% in DMF, 19 mL), addition by synthesizer, 2×1 min at RT.

### Method F (Manual Coupling)

The resin was treated with a 5% solution of Ac₂O in DCM (15 mL) and mixed at RT for 10 min.

After the assembly of the linear peptide the resin was washed with DMF, DCM, diethyl ether, and dried in vacuo. The amino acids and coupling methods are summarized in Table 6.3.

TABLE 6.3

| | Amino | | |
|---|---|---|---|
| Synthesis | acid | | |
| cycle | residue^{a} | Amino acid | SPPS Method |
| 1 | A¹⁰*** | Fmoc-L-Lys(Boc)-OH | A |
| 2 | A¹⁰** | Fmoc-PEG10-OH | A |
| 3 | A¹⁰* | Fmoc-L-K(COpipzaa)(OtBu)-OH | A |
| 4 | A¹⁰ | Fmoc-L-N-Me-Phe(4F)-OH | A |
| 5 | A⁹ | Fmoc-L-N-Me-Dap(Alloc)-OH | B |
| 6 | A⁸ | Fmoc-L-allo-Thr(tBu)-OH | C |
| 7 | A⁷ | Fmoc-L-Trp(Boc)-OH | D |
| 8 | A⁶ | Fmoc-L-Trp(5-OH)-OH | A |
| 9 | A⁵ | Fmoc-L-N-Me-Lys(Ac)-OH | A |
| 10 | A⁴ | Fmoc-L-N-Me-Homo-Phe-OH | B |
| 11 | A³ | Fmoc-L-Ser(Trt)-OH | E |
| 12 | A² | Fmoc-L-Tyr(tBu)-OH | D |
| 13 | A¹ | Fmoc-L-Glu(OAll)-OH | A + Fmoc |
| | | | Deprotection |
| 14 | A¹* | Ac₂O | F (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

### Step 2. Example E2-2

Step 2-1: The resin from Step 1 (calculated with 250 μmol) was suspended with DCM/HFIP (99:1, 15 mL) and treated with phenylsilane (615 μL, 541 mg, 20 eq., 5.0 mmol) and tetrakis(triphenylphosphin) palladium (86.7 mg, 0.3 eq., 0.075 mmol). The suspension was shaken at RT for 1 hr. The solvent was drained, and then the resin was washed with DCM and DMF.

Step 2-2: The resin from Step 2-1 (calculated with 250 μmol) was suspended with DMF (10 mL). Then PyAOP (521 mg, 4.0 eq., 1.0 mmol) and DIPEA (340 μL, 8 eq., 2 mmol) were added. The reaction mixture was stirred at 50° C. for 30 min under microwave irradiation.

Step 2-3: To the resin from Step 2-2 was added TFA/H₂O/DODT/TIS (92.5/2.5/2.5/2.5) (9 mL) and the resin was shaken at RT for 1.5 hr. The resin was removed by filtration. The crude peptide was precipitated with diisopropyl ether/hexane (1/1) (80 mL) giving a precipitate. The suspension was centrifuged, and the solvent was decanted. The product was washed with another portion of diethyl ether. The suspension was centrifuged, and the solvent was decanted. The crude peptide was dried at RT in vacuo.

### Step 3. Example E2

To a solution of the cyclized peptide from Step 2-3 (calculated with 250 μmol) in DMSO (25 mM, 10 mL) were added Biotin-NHS ester (93.9 mg, 1.1 eq., 0.275 mmol) and DIPEA (128 μL, 3 eq., 0.75 mmol). The solution was shaken at RT for 1 hr. The reaction mixture was quenched with AcOH and concentrated in vacuo using EZ-II elite (Genevac, 40° C.). The crude product was purified, and the product was isolated by preparative RP HPLC (Column: XSelect® Peptide CSH C18, OBD™ Prep Column, 130 Å, 5 μm, 19 mm×150 mm; Eluent A: H₂O+0.1% TFA and eluent B: ACN+0.1% TFA). Pure fractions were combined and lyophilized to afford Example E2 (13.35 mg, 4.7 μmol, 1.9% yield, TFA salt) as a white solid. LCMS Method AP-3, t_{R}=4.47 min, [M+2H]²⁺=1357.4.

### 6.3 Synthesis of 2-(4-(((S)-5-((S)-2-((3S,6S,9S,12S,15S,18S,21S,24S,27S)-9-((1H-indol-3-yl)methyl)-15-(4-acetamidobutyl)-27-(5,39-dioxo-43-((3aS,4S,6aR)-2-oxohexahydro-1H-thieno[3,4-d]imidazol-4-yl)-8,11,14,17,20,23,26,29,32,35-decaoxa-4,38-diazatritetracontanamido)-12-((5-hydroxy-1H-indol-3-yl)methyl)-24-(4-hydroxybenzyl)-6-((S)-1-hydroxyethyl)-21-(hydroxymethyl)-N,4,16,19-tetramethyl-5,8,11,14,17,20,23,26,30-nonaoxo-18-phenethyl-1,4,7,10,13,16,19,22,25-nonaazacyclotriacontane-3-carboxamido)-3-(4-fluorophenyl)propanamido)-6-amino-6-oxohexyl)carbamoyl)piperazin-1-yl) acetic acid TFA salt (Example E3 (E3 disclosed as SEQ ID NO: 86))

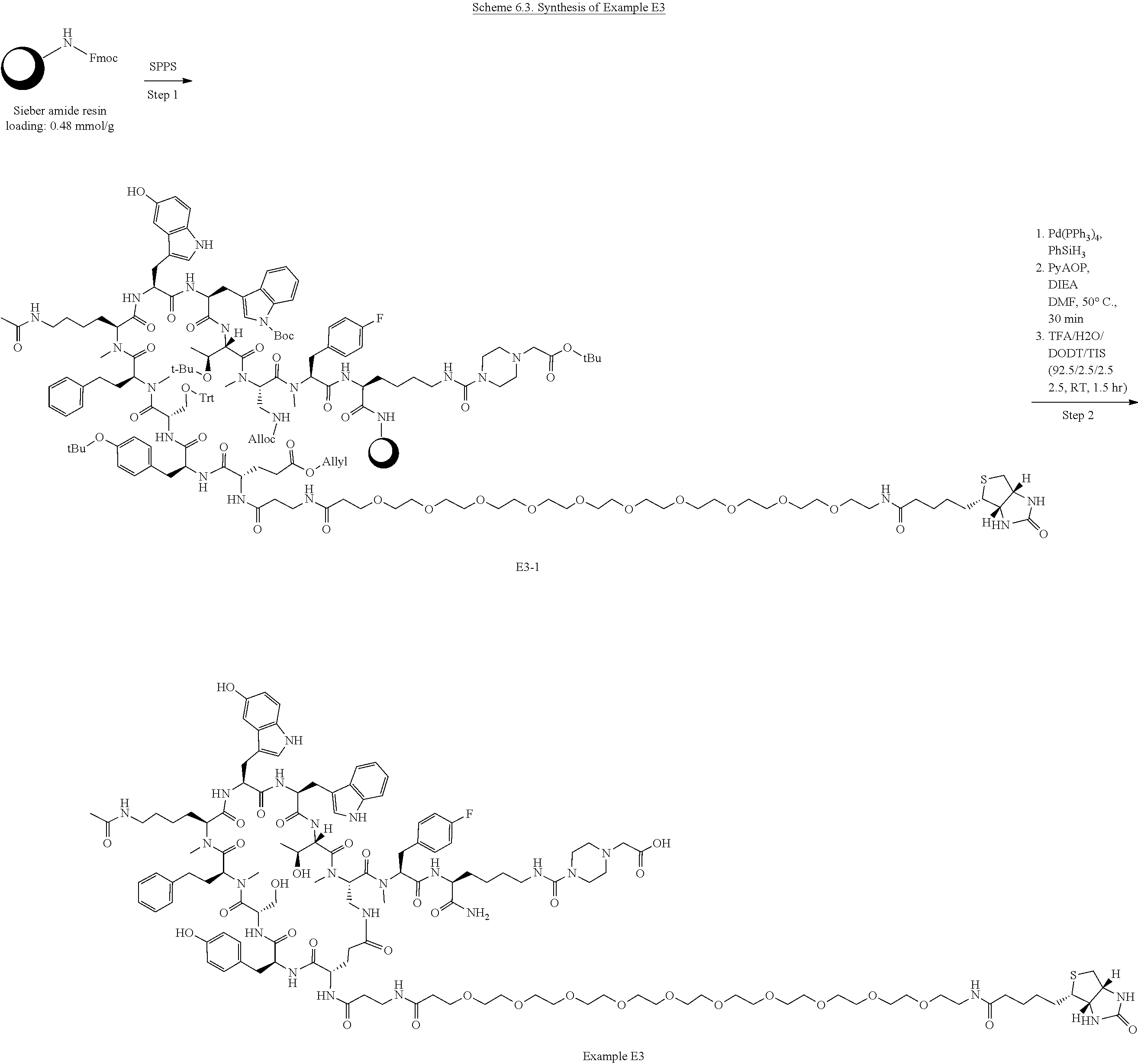

### Step 1. E3-1

The assembly of the linear peptide was performed on the CEM Liberty Blue Synthesizer and by manual coupling using commercial Sieber amide resin (loading 0.48 mmol/g, 521 mg, 0.25 mmol). The couplings were carried out using the following solutions: Fmoc-amino acid (0.21 M solution DMF, 5 mL, 4.2 eq.), DIC (1 M solution in DMF, 2 mL, 8 eq.), Oxyma Pure® (0.5 M solution in DMF, 2 mL, 4 eq.), addition by synthesizer. The Fmoc removal was performed using a solution of pyrrolidine (10% in DMF, 10 mL) or 53 mM Oxyma Pure® in pyrrolidine (5.3% in DMF, 19 mL), addition by synthesizer.

The amino acids were coupled with a set of different methods.
Method A Fmoc removal: 1×1 min at 90° C./Coupling: 1×3 min at 90° C.
Method B Fmoc removal: 1×1 min at 90° C./Coupling: 2×3 min at 90° C.
Method C Fmoc removal: 2×1 min at RT/Coupling: 2×30 min at 75° C.
Method D Fmoc removal: 2×1 min at RT/Coupling: 1×3 min at 90° C.
Method E Fmoc removal: 2×1 min at RT/Coupling: 2×3 min at 90° C.

Fmoc deprotection (after Synthesis cycle 13) was performed using a solution of pyrrolidine (10% in DMF, 10 mL) or 53 mM Oxyma Pure® in pyrrolidine (5.3% in DMF, 19 mL), addition by synthesizer, 1×1 min at RT.

### Method F (Manual Coupling)

The resin was treated with Biotin-NHS ester (341 mg, 4 eq., 1 mmol) and DIPEA (213 μL, 5 eq., 1.25 mmol) in DMSO/DMF (1:1, 15 mL) and mixed at RT for 4 hr.

After the assembly of the linear peptide the resin was washed with DMF, DCM, diethyl ether, and dried in vacuo. The amino acids and coupling methods are summarized in Table 6.4.

TABLE 6.4

| | Amino | | |
|---|---|---|---|
| Synthesis | acid | | |
| cycle | residue^{a} | Amino acid | SPPS Method |
| 1 | A¹⁰* | Fmoc-L-K(Copipzaa)(OtBu)-OH | A |
| 2 | A¹⁰ | Fmoc-L-N-Me-Phe(4F)-OH | A |
| 3 | A⁹ | Fmoc-L-N-Me-Dap(Alloc)-OH | B |
| 4 | A⁸ | Fmoc-L-allo-Thr(tBu)-OH | C |
| 5 | A⁷ | Fmoc-L-Trp(Boc)-OH | D |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(Trt)-OH | E |
| 10 | A² | Fmoc-L-Tyr(tBu)-OH | D |
| 11 | A¹ | Fmoc-L-Glu(OAll)-OH | A |
| 12 | A¹* | Fmoc-L-Beta-Ala-OH | D |
| 13 | A¹** | Fmoc-PEG10-OH | A + Fmoc |
| | | | deprotection |
| 14 | A¹*** | Biotin-NHS | F (Manual |
| | | | Coupling) |

^{a}Refers to the corresponding amino acid residue of cyclic peptide, P, as found, e.g., in Formulae (I), (Ia), (Ib), (Ic), (Id), or in the Table of Example III.

### Step 2. Example E3

Step 2-1: The resin from Step 1 (calculated with 250 μmol) was suspended with DCM/HFIP (99:1, 15 mL) and treated with phenylsilane (615 μL, 541 mg, 20 eq., 5.0 mmol) and tetrakis(triphenylphosphin) palladium (86.7 mg, 0.3 eq., 0.075 mmol). The suspension was shaken at RT for 1 hr. The solvent was drained, and then the resin was washed with DCM and DMF.

Step 2-2: The resin from Step 2-1 (calculated with 250 μmol) was suspended with DMF (10 mL). Then PyAOP (521 mg, 4.0 eq., 1.0 mmol) and DIPEA (340 μL, 8 eq., 2 mmol) were added. The reaction mixture was stirred at 50° C. for 30 min under microwave irradiation.

Step 2-3: To the resin from Step 2-2 was added TFA/H₂O/DODT/TIS (92.5/2.5/2.5/2.5) (9 mL) and the resin was shaken at RT for 1.5 hr. The resin was removed by filtration. The crude peptide was precipitated with diisopropyl ether/hexane (1/1) (80 mL) giving a precipitate. The suspension was centrifuged, and the solvent was decanted. The product was washed with another portion of diethyl ether. The suspension was centrifuged, and the solvent was decanted. The crude peptide was dried at RT under vacuo. The crude product was purified, and the product was isolated by preparative RP HPLC twice (1; Column: XSelect® Peptide CSH C18, OBD™ Prep Column, 130 Å, 5 μm, 19 mm×150 mm; Eluent A: H₂O+0.1% TFA and eluent B: ACN+0.1% TFA, and then 2; Column: Kinetex® EVO C18, OBD™ Prep Column, 130 Å, 5 μm, 30 mm×150 mm; Eluent A: H₂O+1% AcOH and eluent B: ACN+1% AcOH). Pure fractions were combined. TFA (170 μL) was added. The resulting solution was lyophilized to afford Example E3 (3.25 mg, 1.2 μmol, 0.5% yield, TFA salt) as a white solid. LCMS Method AP-3, t_{R}=4.49 min, [M+2H]²⁺=1307.9.

### 6.4 Synthesis of (3S,6S,9S,12S,15S,18S,21S,24S,27S)-9-((1H-indol-3-yl)methyl)-27-acetamido-15-(4-acetamidobutyl)-N-((2S,5S)-5-carbamoyl-1-(4-fluorophenyl)-3,11,45-trioxo-49-((3aS,4S,6aR)-2-oxohexahydro-1H-thieno[3,4-d]imidazol-4-yl)-14,17,20,23,26,29,32,35,38,41-decaoxa-4,10,44-triazanonatetracontan-2-yl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide (Example E4 (A76 disclosed as SEQ ID NO: 34 and E4 disclosed as SEQ ID NO: 87))

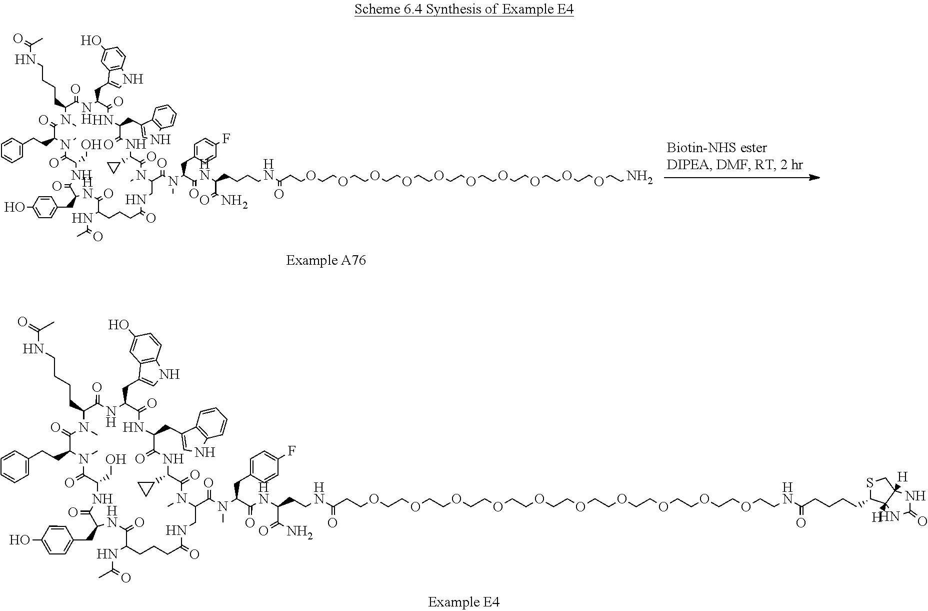

To a clear colorless solution of Example A76 (8 mg, 1 eq., 3.459 μmol, TFA salt) in DMF (0.5 mL) at RT were added Biotin-NHS ester (1.77 mg, 1.5 eq., 5.189 μmol) and DIPEA (2.236 mg, 3.01 μL, 5 eq., 17.3 μmol). The resulting clear solution was stirred at RT for 2 hr. The reaction mixture was quenched with ACN/water (1/1) and the product was isolated by preparative RP HPLC (Column: XSelect® Peptide CSH C18, OBD™ Prep Column, 130 Å, 5 μm, 30×250 mm; Eluent A: H₂O+0.1% TFA and eluent B: ACN). Pure fractions were combined and lyophilized to afford Example E4 (6.9 mg, 2.8 μmol, 81% yield) as a white solid. LCMS Method AP-1, t_{R}=4.69 min, [M+H]⁺=2425.2.

The following examples (for structures, see Table 6.5) were synthesized in analogy to Example E4:

Example E5 was synthesized from Example A77.

TABLE 6.5
Examples synthesized in analogy to Example E4 (Scheme 6.4).

| Ex. No. | Structure/Example Name | LCMS |
|---|---|---|
| E5 (SEQ ID NO: 88) | 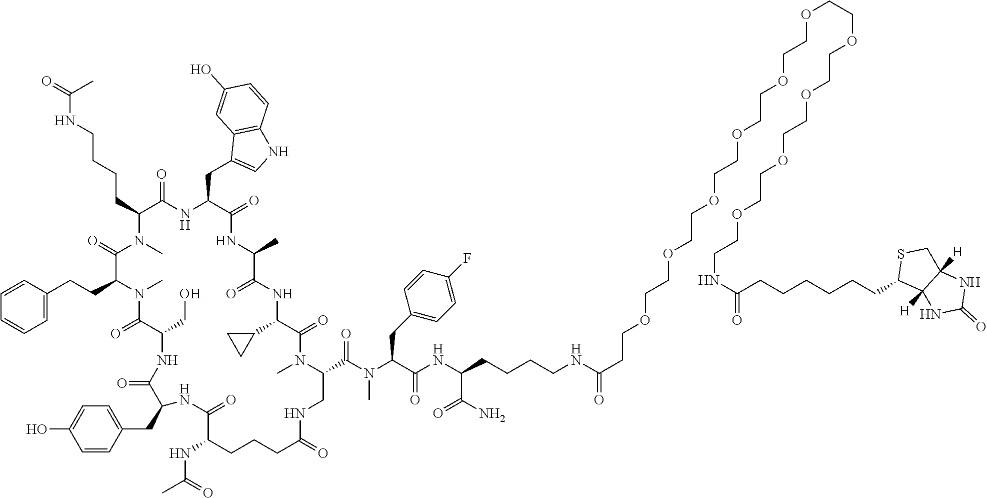 | Method AP-1 t_{R} = 4.32 min [M + H]⁺ = 2310.2 |
| | (3S,6S,9S,12S,15S,18S,21S,24S,27S)-27-acetamido-15-(4-acetamidobutyl)-N-((2S,5S)-5-carbamoyl-1-(4-fluorophenyl)- | |
| | 3,11,45-trioxo-49-((3aS,4S,6aR)-2-oxohexahydro-1H-thieno[3,4-d]imidazol-4-yl)-14,17,20,23,26,29,32,35,38,41- | |
| | decaoxa-4,10,44-triazanonatetracontan-2-yl)-6-cyclopropyl-12-((5-hydroxy-1H-indol-3-yl)methyl)-24-(4- | |
| | hydroxybenzyl)-21-(hydroxymethyl)-N,4,9,16,19-pentamethyl-5,8,11,14,17,20,23,26,31-nonaoxo-18-phenethyl- | |
| | 1,4,7,10,13,16,19,22,25-nonaazacyclohentriacontane-3-carboxamide | |

### 7 Varia 1 (“F” Examples)

### 7.1 Synthesis of (3S,6S,9S,12S,15S,18S,21S,24S,27S)-9-((1H-indol-3-yl)methyl)-27-acetamido-15-(4-acetamidobutyl)-N—((S)-1-(((S)-1-amino-6-(6-fluoronicotinamido)-1-oxohexan-2-yl)amino)-3-(4-fluorophenyl)-1-oxopropan-2-yl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide (Example F1 (A40 disclosed as SEQ ID NO: 14 and F1 disclosed as SEQ ID NO: 89))

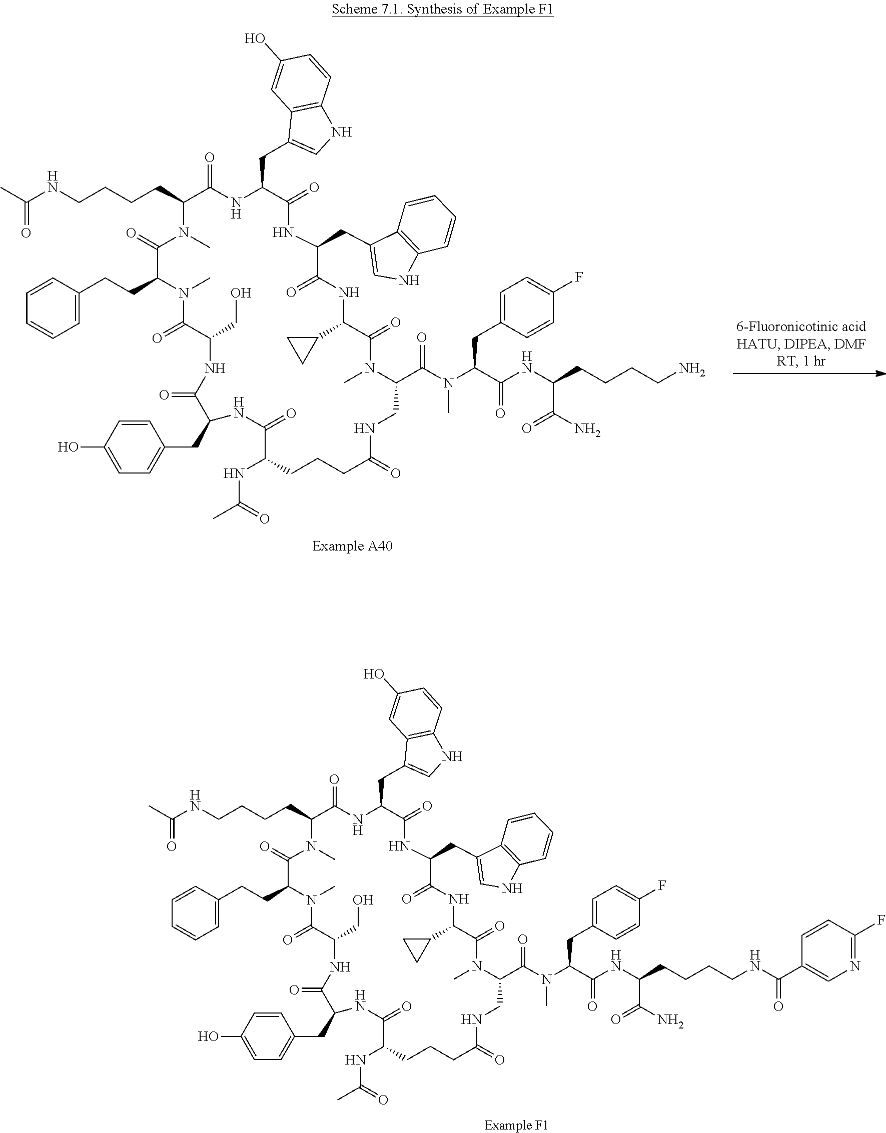

To a clear colorless solution of Example A40 (27 mg, 1 eq., 14.99 μmol, TFA salt) in DMF (0.5 mL) at RT was added a solution of 6-fluoronicotinic acid (CAS 403-45-2, 3.2 mg, 1.5 eq., 22.49 μmol), HATU (7.98 mg, 1.4 eq., 20.99 μmol) and DIPEA (97 mg, 13.1 μL, 5 eq., 74.96 μmol) in DMF (0.5 mL) (pre-activated at RT for 5 min). The reaction mixture was stirred at RT for 1 hr. The reaction mixture was quenched with ACN/water (1/1), and the product was isolated by preparative RP HPLC (Column: XBridge BEH C18 OBD Prep Column, 130 Å, 5 μm, 30 mm×150 mm; 30 mL/min; Eluent A: H₂O+0.08% NH₄HCO₃ and eluent B: ACN). Pure fractions were combined and lyophilized to afford Example F1 (19 mg, 10 μmol, 69% yield) as a white solid. LCMS Method AP-1, t_{R}=4.45 min, [M+H]⁺=1809.9.

### 7.2 Synthesis of (3S,6S,9S,12S,15S,18S,21S,24S,27S)-9-((1H-indol-3-yl)methyl)-27-acetamido-15-(4-acetamidobutyl)-N—((S)-1-(((S)-1-amino-6-(6-fluoronicotinamido)-1-oxohexan-2-yl)amino)-3-(4-fluorophenyl)-1-oxopropan-2-yl)-6-cyclopropyl-12-((5-hydroxy-1H-indol-3-yl)methyl)-24-(4-hydroxybenzyl)-21-(hydroxymethyl)-N,4,16,19-tetramethyl-5,8,11,14,17,20,23,26,31-nonaoxo-18-phenethyl-1,4,7,10,13,16,19,22,25-nonaazacyclohentriacontane-3-carboxamide TFA salt (Example F2 (A40 disclosed as SEQ ID NO: 14 and F2 disclosed as SEQ ID NO: 90))

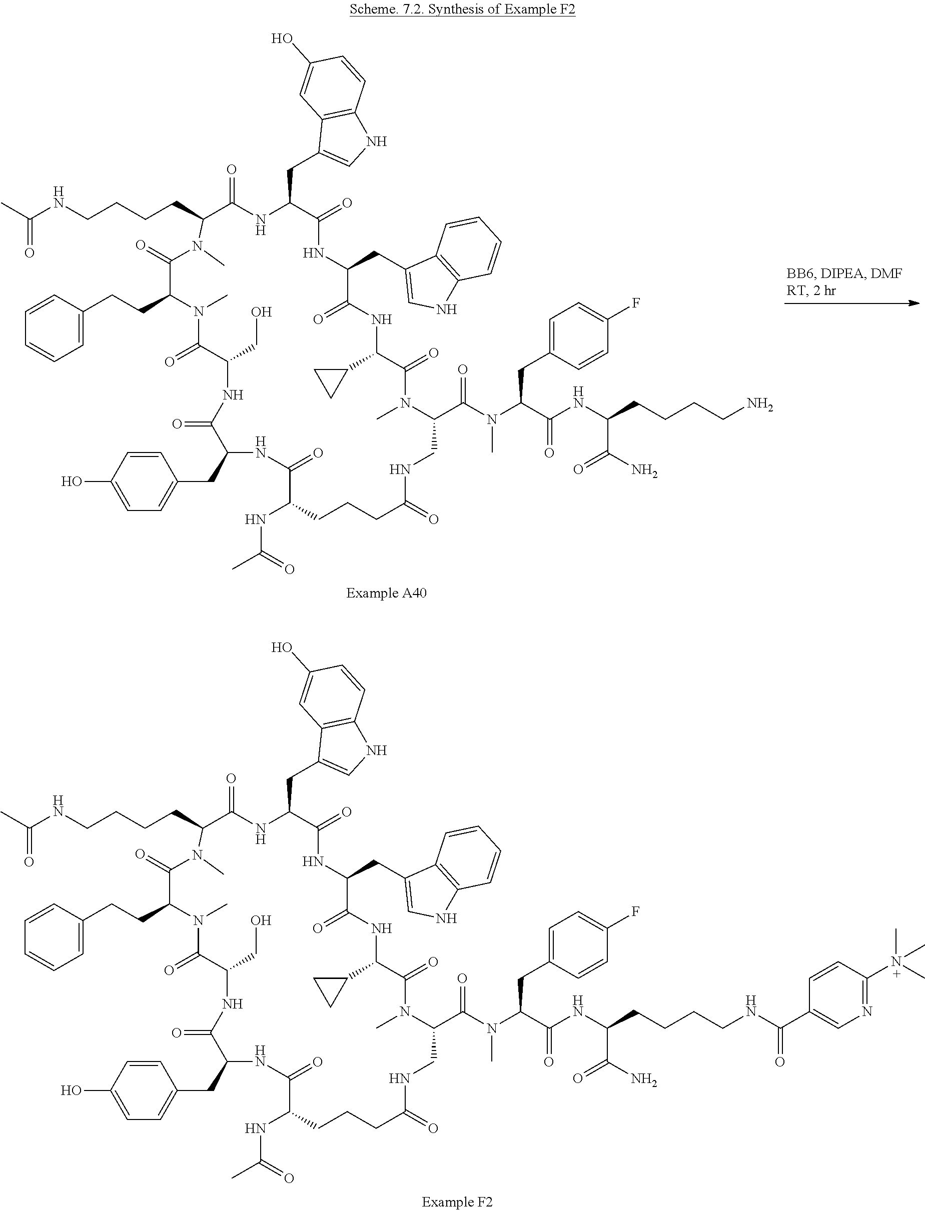

To a clear colorless solution of Example A40 (51.8 mg, 1 eq., 28.76 μmol, TFA salt) and DIPEA (11.15 mg, 15 μL, 3 eq., 86.29 μmol) in DMF (1 mL) at RT was added BB6 (16.51 mg, 1.2 eq., 34.51 μmol). The reaction mixture was stirred at RT for 2 hr. The reaction mixture was precipitated with cold MTBE (40 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford Example F2 (51.5 mg, 26 μmol, 90% yield, TFA salt) as a white solid. LCMS Method AP-1, t_{R}=3.99 min, [M+H]⁺=1850.9.

The following examples (for structures, see Table 7.1) were synthesized in analogy to Example F1 and Example F2:

Example F3 was synthesized from Example A43 in analogy to Example F1.

Example F4 was synthesized from Example A43 in analogy to Example F2.

TABLE 7.1
Examples synthesized in analogy to Example F1 (Scheme 7.1) and Example
F2 (Scheme 7.2)

| Ex. No. | Structure/Example Name | LCMS |
|---|---|---|
| F3 (SEQ ID NO: 91) | 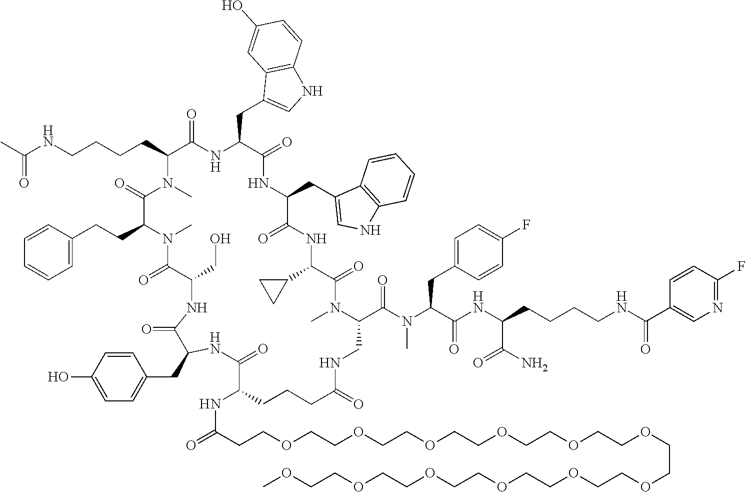 [SMILES: COCCOCCOCCOCCOCCOCCOCCOCCOCCOCCOCCOCCC(=O)N[C@H]1CCCC(=O)NC[C@@H](C(=O)N(C)[C@@H](Cc2ccc(F)cc2)C(=O)N[C@@H](CCCCNC(=O)c2ccc(F)nc2)C(N)=O)N(C)C(=O)[C@H](C2CC2)NC(=O)[C@H](Cc2c[nH]c3ccccc23)NC(=O)[C@H](Cc2c[nH]c3ccc(O)cc23)NC(=O)[C@H](CCCCNC(C)=O)N(C)C(=O)[C@H](CCc2ccccc2)N(C)C(=O)[C@H](CO)NC(=O)[C@H](Cc2ccc(O)cc2)NC1=O] | Method AP-1 t_{R} = 4.83 min [M + H]⁺ = 2339.2 |
| | (3S,6S,9S,12S,15S,18S,21S,24S,27S)-9-((1H-indol-3-yl)methyl)-15-(4- | |
| | acetamidobutyl)-N-((S)-1-(((S)-1-amino-6-(6-fluoronicotinamido)-1-oxohexan- | |
| | 2-yl)amino)-3-(4-fluorophenyl)-1-oxopropan-2-yl)-6-cyclopropyl-12-((5- | |
| | hydroxy-1H-indol-3-yl)methyl)-24-(4-hydroxybenzyl)-21-(hydroxymethyl)- | |
| | N,4,16,19-tetramethyl-27-(2,5,8,11,14,17,20,23,26,29,32,35- | |
| | dodecaoxaoctatriacontan-38-amido)-5,8,11,14,17,20,23,26,31-nonaoxo-18- | |
| | phenethyl-1,4,7,10,13,16,19,22,25-nonaazacyclohentriacontane-3-carboxamide | |
| F4 (SEQ ID NO: 92) | 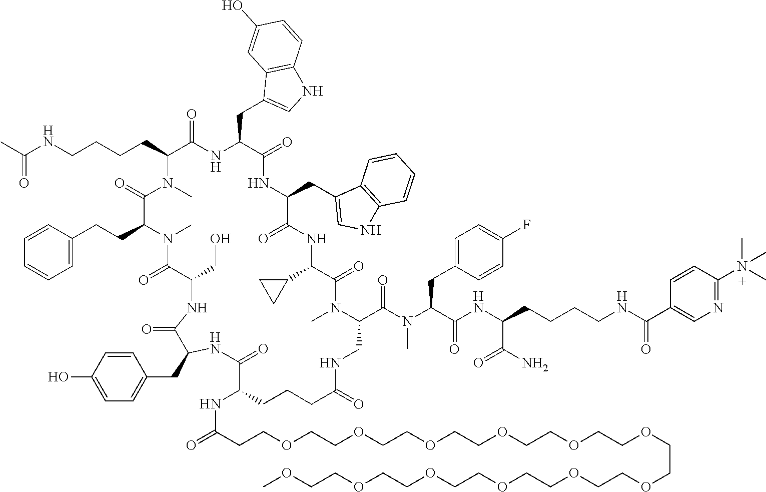 | Method AP-1 t_{R} = 4.37 min [M + H]⁺ = 2379.2 |
| | 5-(((S)-5-((S)-2-((3S,6S,9S,12S,15S,18S,21S,24S,27S)-9-((1H-indol-3- | |
| | yl)methyl)-15-(4-acetamidobutyl)-6-cyclopropyl-12-((5-hydroxy-1H-indol-3- | |
| | yl)methyl)-24-(4-hydroxybenzyl)-21-(hydroxymethyl)-N,4,16,19-tetramethyl- | |
| | 27-(2,5,8,11,14,17,20,23,26,29,32,35-dodecaoxaoctatriacontan-38-amido)- | |
| | 5,8,11,14,17,20,23,26,31-nonaoxo-18-phenethyl-1,4,7,10,13,16,19,22,25- | |
| | nonaazacyclohentriacontane-3-carboxamido)-3-(4-fluorophenyl)propanamido)- | |
| | 6-amino-6-oxohexyl)carbamoyl)-N,N,N-trimethylpyridin-2-aminium TFA salt | |

### 8 Varia 2 (“G” Examples)

### 8.1 Synthesis of Example G1 (G1-4 Disclosed as SEQ ID NO: 119 and G1 Disclosed as SEQ ID NO: 93)

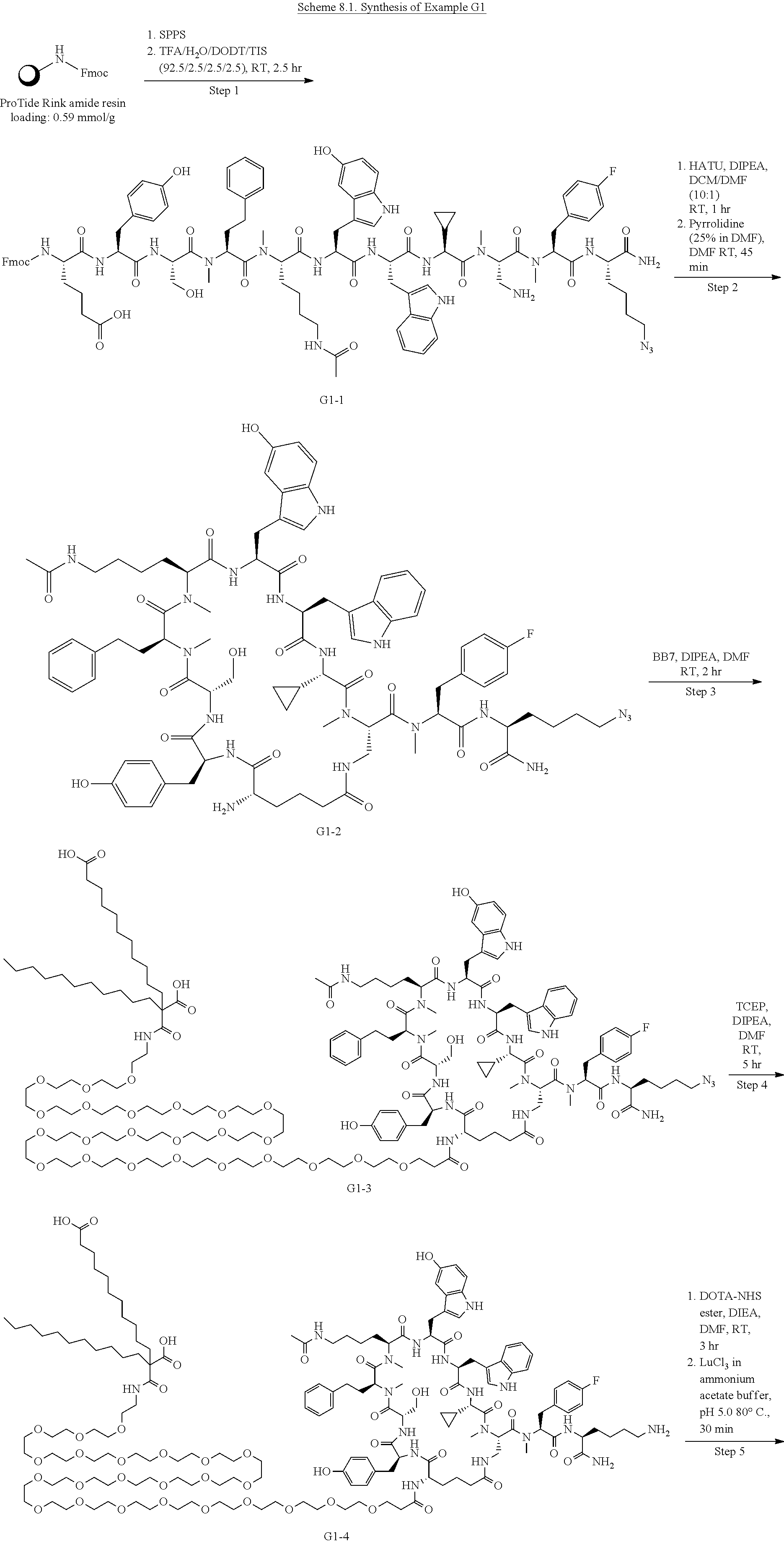

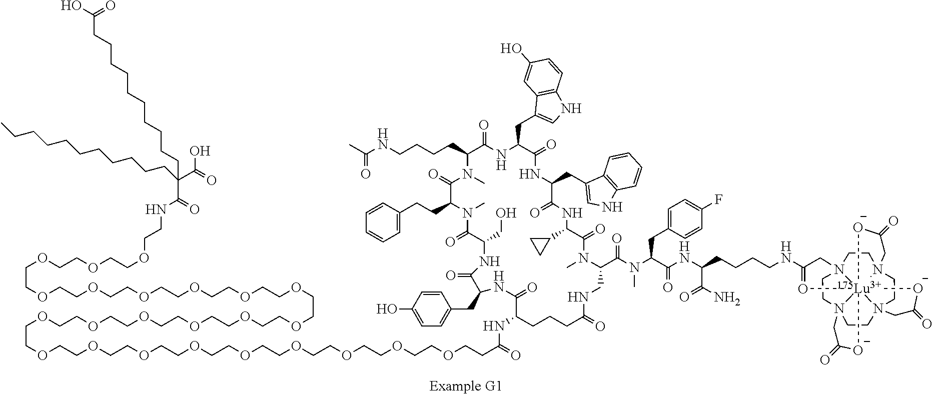

### Step 1. G1-1

Step 1-1: The assembly of the linear peptide was done on the CEM Liberty Prime Synthesizer and by manual coupling using commercial Rink amide resin R1 (loading 0.59 mmol/g, 508 mg, 0.300 mmol). The couplings were carried out using the following solutions: Fmoc-amino acid (0.5 M solution DMF, 2.5 mL, 4.2 eq.), DIC (4 M solution in DMF, 0.63 mL, 8.4 eq.), Oxyma Pure® (0.25 M solution in DMF, 5 mL, 4.2 eq.), addition by synthesizer. Fmoc removal was performed using a solution of pyrrolidine (25% in DMF, 1.5 mL), addition by synthesizer.

The amino acids were coupled with a set of different methods.
Method A Fmoc removal: 1×1 min at 110° C./Coupling: 1×2 min at 105° C.
Method B Fmoc removal: 2×10 min at RT/Coupling: 2×2 min at 105° C.

Fmoc deprotection (after Synthesis cycle 10) was performed using 25% pyrrolidine in DMF (1.5 mL), addition by synthesizer, 1×1 min at 110° C.

### Method C (Manual Coupling)

The resin was treated with a pre-activated solution of Fmoc-L-Homo-Glu(OtBu)-OH (198 mg, 1.5 eq., 0.45 mmol), HATU (228 mg, 2 eq., 0.60 mmol) and DIPEA (194 mg, 261 μL, 5 eq., 1.5 mmol) in DMF (6 mL) and mixed at RT for 2 hr.

After the assembly of the linear peptide the resin was washed with DMF (5×) and DCM (5×). The amino acids and coupling methods are summarized in Table 8.1.

TABLE 8.1

| Synthesis | Amino acid | | |
|---|---|---|---|
| cycle | residue^{a} | Amino acid | Method |
| 1 | A¹⁰* | Fmoc-L-Lys(N3)-OH | A |
| 2 | A¹⁰ | Fmoc-L-N-Me-Phe(4F)-OH | A |
| 3 | A⁹ | Fmoc-L-N-Me-Dap(Boc)-OH | B |
| 4 | A⁸ | Fmoc-L-CyclopropylGly-OH | B |
| 5 | A⁷ | Fmoc-L-Trp(Boc)-OH | A |
| 6 | A⁶ | Fmoc-L-Trp(5-OH)-OH | A |
| 7 | A⁵ | Fmoc-L-N-Me-Lys(Ac)-OH | A |
| 8 | A⁴ | Fmoc-L-N-Me-Homo-Phe-OH | B |
| 9 | A³ | Fmoc-L-Ser(tBu)-OH | B |
| 10 | A² | Fmoc-L-Tyr(tBu)-OH | B + Fmoc |
| | | | deprotection |
| 11 | A¹ | Fmoc-L-Homo-Glu(OtBu)-OH | C (Manual |
| | | | coupling) |

^{a}Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added TFA/H₂O/DODT/TIS (92.5/2.5/2.5/2.5) (15 mL) and the resin was shaken at RT for 2.5 hr. The crude peptide was precipitated with cold diethyl ether (160 mL). The suspension was centrifuged, and the solvent was decanted. The product was washed with another portion of cold diethyl ether (120 mL). The suspension was centrifuged, and the solvent was decanted. The crude peptide was dried at RT under vacuo, and the product was isolated by preparative RP HPLC (Column: XSelect® Peptide CSH C18, OBD™ Prep Column, 130 Å, 5 μm, 30×250 mm; Eluent A: H₂O+0.1% TFA and eluent B: ACN). Pure fractions were combined and lyophilized to afford G1-1 (110 mg, 54 μmol, 18% yield, TFA salt) as a white solid. LCMS Method AP-1, t_{R}=4.96 min, [M+H]⁺=1911.9.

### Step 2. G1-2

Step 2-1: G1-1 (110 mg, 1 eq., 57 μmol, TFA salt) was dissolved in DCM (30 mL) and DMF (3 mL). Then HATU (22.7 mg, 1.1 eq., 59.8 μmol), followed by DIPEA (35.1 mg, 47.3 μL, 5 eq., 271.6 μmol) were added. The reaction mixture was stirred at RT for 1 hr, then concentrated at 30° C. in vacuo to remove the DCM.

Step 2-2: The residue from Step 2-1 was treated with pyrrolidine (25% in DMF) (154.5 mg, 178 μL, 10 eq., 543.2 μmol) and shaken at RT for 45 min. The reaction mixture was directly purified, and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford G1-2 (31.7 mg, 17 μmol, 32% yield, TFA salt) as a white solid. LCMS Method AP-1, t_{R}=4.29 min, [M+H]⁺=1670.8.

### Step 3. G1-3

G1-2 (31.7 mg, 1 eq., 17.76 μmol, TFA salt) was dissolved in DMF (1 mL), then BB7 (44.4 mg, 1.5 eq., 26.64 μmol) and DIPEA (11.5 mg, 15.5 μL, 5 eq., 88.8 μmol) were added. The reaction mixture was shaken at RT for 2 hr. The reaction mixture was quenched with ACN/water (1/1) and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford G1-3 (40 mg, 11 μmol, 64% yield) as a white solid. LCMS Method AP-1, t_{R}=7.43 min, [M+2H]²⁺=1612.4.

### Step 4. G1-4

G1-3 (40 mg, 1 eq., 12.41 μmol) was dissolved in DMF (1.500 mL), then TCEP (17.8 mg, 5 eq., 62.04 μmol, HCl salt) and DIPEA (16 mg, 21.6 μL, 10 eq., 124.1 μmol) were added. The reaction mixture was shaken at RT for 3 hr. The reaction mixture was quenched with ACN/water (1/1) and the product was isolated by preparative RP HPLC (Column: XSelect® Peptide CSH C18, OBD™ Prep Column, 130 Å, 5 μm, 30 mm×250 mm; Eluent A: H₂O+0.1% TFA and eluent B: ACN). Pure fractions were combined and lyophilized to afford G1-4 (18.2 mg, 5.4 μmol, 44% yield, TFA salt) as a white solid. LCMS Method AP-1, t_{R}=6.49 min, [M+2H]²⁺=1599.9.

### Step 5. Example G1

Step 5-1: G1-4 (18 mg, 1 eq., 5.435 μmol, TFA salt) was dissolved in DMF (1 mL), then DOTA-NHS ester (8.3 mg, 2 eq., 10.87 μmol, hexafluorophosphate-trifluoroacetate) and DIPEA (3.5 mg, 4.73 μL, 5 eq., 27.17 μmol) were added. The reaction mixture was shaken at RT for 3 hr.

Step 5-2: The reaction mixture from Step 5-1 was diluted with ammonium acetate buffer pH 5.0 (1.000 mL). Then 100 mM LuCl₃ in ammonium acetate buffer pH 5.0 (271.7 mL, 100 mM, 5 eq., 27.17 μmol) was added. The reaction mixture was shaken at 80° C. for 30 min. The reaction mixture was quenched with ACN/water (1/1) and the product was isolated by preparative RP HPLC (Column: XSelect® Peptide CSH C18, OBD™ Prep Column, 130 Å, 5 μm, 30 mm×100 mm; Eluent A: H₂O+0.1% TFA and eluent B: ACN). Pure fractions were combined and lyophilized to afford Example G1 (15.6 mg, 4.1 μmol, 76% yield) as a white solid. LCMS Method AP-1, t_{R}=6.94 min, [M+2H]²⁺=1879.0.

### 9 Radiolabeling

### 9.1 General Procedures

The compounds of the present invention can be radiolabeled using a variety of methods, which lead to high radiochemical yields and purities of the final products. The following methods represent a selection of possible radiolabeling processes suitable for the chelator-conjugated compounds disclosed herein. Other variations of the radiolabeling processes (e.g., different salts of the precursors, different labeling buffers, reaction pH's, temperatures, radiometals) are also valid and should result in similar intermediate and final products.

For radiolabeling with [¹⁷⁷Lu]LuCl₃, [¹⁷⁷Lu]LuCl₃ in 0.04 M HCl, as received from the supplier (68-1300 MBq), was diluted in labeling buffer (a combination of, e.g., acetic acid/ascorbic acid/sodium acetate) at a preferable pH from 4.3 to 5.5. A solution containing the DOTA-conjugated compound (0.9-32.4 nmol in a minimum volume) was subsequently added. The reaction mixture was heated for 15 min at 95° C. and then cooled down. Finally, the product was diluted in formulation solution to reach the desired activity concentration (370-740 MBq/mL). The details of each radiolabeling method with [¹⁷⁷Lu]LuCl₃ are specified in 9.3.

For radiolabeling with [⁶⁸Ga]GaCl₃, [⁶⁸Ga]GaCl₃ in HCl 0.1 M (370 MBq) was obtained from a ⁶⁸Ge/⁶⁸Ga generator and eluted directly into a glass vial containing the labeling buffer (a combination of, ascorbic acid and sodium acetate) and a solution of the DOTA-conjugated compound (30 nmol in a minimum volume) at a preferable pH from 3.5 to 3.9. The reaction mixture was heated for 15 min at 97° C. and then cooled down. The details for the radiolabeling method with [⁶⁸Ga]GaCl₃ are specified in Section 9.4.

For radiolabeling with Ac-225, the radioisotope in the solid form was first dissolved on-site in 0.04 M HCl to an activity concentration of 50 MBq/mL. Then, the [²²⁵Ac]AcCl₃ solution was mixed in labeling buffer at pH 8.0 followed by the addition of the needed DOTA-conjugated compound to reach the desired molar activity. The reaction mixture was heated for 20 min at 95° C. cooled down, diluted with sodium ascorbate, gentisic acid, and 0.9% saline solutions to achieve the desired target activity concentration. The details for the radiolabeling method with [²²⁵Ac]AcCl₃ are specified in Section 9.5.

### 9.2 Analytical Methods

Radio-HPLC Analytical Method: Eluent A: Water+0.1% TFA; Eluent B: ACN+0.1% TFA; Column temperature: 25° C.; Flow: 1.0 mL/min; Column: Luna 5 μm C18 (2) 100 Å, LC Column 150×4.6 mm; Gradient: hold 10% B for 1 min, from 10 to 80% B in 8 min, hold 80% B for 2 min, from 80 to 15% B in 1 min.

Radio-iTLC Analytical Method: Performed on glass microfiber chromatography paper impregnated with silica gel (Agilent iTLC-SG SGI0001). For Lu-177 and Ga-68, the radiotracer-spotted iTLC plates were directly read in a radio-TLC scanner after developing in a sodium citrate 0.1M (pH 4.6) mobile phase. Rf([¹⁷⁷Lu/⁶⁸Ga]Lu/Ga-labeled compounds)=0.0-0.5 and Rf (unchelated ¹⁷⁷Lu³⁺/⁶⁸Ga³⁺)=0.5-1.0.

For Ac-225, the radiotracer-spotted TLC-plates were developed in an ammonium acetate 3M/methanol (20/80) mobile phase and measured, after at least 16 hours, using a phosphor imaging technique. Rf([²²⁵Ac]Ac-labeled compounds)=0.5-1.0 and Rf (unchelated ²²⁵Ac³⁺)=0.0-0.5.

9.3 Radiolabeling with [¹⁷⁷Lu]LuCl₃ (“H” Examples)

### 9.3.1 Method 1

### Materials:

| Name | Supplier |
|---|---|
| Ethanol absolute ≥99.9% for analysis EMSURE ® ACS, ISO, Reag. Ph Eur | Merck |
| Acetic acid (glacial) 100% anhydrous for analysis EMSURE ® ACS | Merck |
| Hydrochloric acid 30% for inorganic trace analysis | Merck |
| Non-carrier added [¹⁷⁷Lu]LuCl₃ in 0.04M hydrochloric acid solution | DSD Pharma |
| | GmbH |
| Sodium acetate trihydrate for analysis EMSURE ® ACS, ISO, Reag. Ph Eur | Merck |
| Ascorbic acid ≥99.9998% (metals basis), TraceSELECT ™, Fluka ™ | Honeywell |
| Water TraceSELECT ™, for trace analysis | Honeywell |
| Phosphate Buffered Saline w/o Calcium w/o Magnesium w/o Potassium | VWR |
| Chloride | |

Labeling buffer: The labeling buffer consists of ascorbic acid (7.14 mg/mL), sodium acetate (204.0 mg/mL), and ethanol (27.6 mg/mL) in TraceSELECT™ water, adjusted to pH 4.7 with acetic acid. The labeling buffer was stored at 4-8° C.

Formulation solution (5% EtOH in PBS): The formulation solution was prepared by mixing 2.5 mL ethanol in 47.5 mL Phosphate Buffered Saline. The formulation solution was stored at 4-8° C.

Precursor stock solution for radiolabeling (1 mM): The DOTA precursors were dissolved in 1.0 mL ethanol in TraceSELECT™ water. The solution was fractionated and stored in a freezer at −20° C.

Radiolabeling (37 MBg/nmol): In a low protein binding tube, 50 μL [¹⁷⁷Lu]LuCl₃ in 0.04 M HCl (120 MBq) was diluted in 106.5 μL labeling buffer. After that, 3.2 nmol of the DOTA-precursor stock solution (1 mM) was added to the reaction mixture to fulfill a molar activity of 37 MBq/nmol. This reaction mixture, with a pH of approx. 4.7, was left to stir (450 rpm) at 95° C. for 15 min in a shielded Thermoshaker Incubator. After cooling at room temperature, the completion of the reaction was confirmed by radio-iTLC and radio-HPLC and there was no need for further purification steps. The final product was diluted with formulation solution to an activity concentration of 370 MBq/mL.

### 9.3.2 Method 2

### Materials:

| Name | Supplier |
|---|---|
| Ethanol absolute ≥99.9% for analysis EMSURE ® ACS, ISO, Reag. Ph Eur | Merck |
| Hydrochloric acid 30% for inorganic trace analysis | Merck |
| Non-carrier added [¹⁷⁷Lu]LuCl₃ in 0.04M hydrochloric acid solution | DSD Pharma |
| | GmbH |
| Sodium acetate trihydrate for analysis EMSURE ® ACS, ISO, Reag. Ph Eur | Merck |
| Ascorbic acid ≥99.9998% (metals basis), TraceSELECT ™, Fluka ™ | Honeywell |
| Water TraceSELECT ™, for trace analysis | Honeywell |
| Phosphate Buffered Saline w/o Calcium w/o Magnesium w/o Potassium | |
| Chloride | |
| Kolliphor ® HS 15 | Merck |

Labeling buffer: The labeling buffer consists of ascorbic acid (140.9 mg/mL) and sodium acetate (136.1 mg/mL) in TraceSELECT™ water. The labeling buffer was stored at 4-8° C.

Formulation solution (Kolliphor in PBS with 5% EtOH): The formulation solution was prepared by mixing Kolliphor HS15 (0.3 mg/ml) with PBS containing 5% ethanol. The formulation solution was stored at 4-8° C.

Precursor stock solution for radiolabeling (1 mM): The DOTA precursors were dissolved in 1.0 mL ethanol in TraceSELECT™ water. The solution was fractionated and stored in a freezer at −20° C.

Radiolabeling (37-74 MBq/nmol): In a low protein binding tube, 30-50 μL [¹⁷⁷Lu]LuCl₃ in 0.04 M HCl (68-1200 MBq) was diluted in 60-120 μL labeling buffer. After that, 0.9-32.4 nmol of the DOTA-precursor stock solution (1 mM) was added to the reaction mixture to fulfill a molar activity of 37-74 MBq/nmol. This reaction mixture, with a pH of approx. 4.3, was left to stir (450 rpm) at 95° C. for 15 min in a shielded Thermoshaker Incubator. After cooling at room temperature, the completion of the reaction was confirmed by radio-iTLC and radio-HPLC and there was no need for further purification steps. The final product was diluted with formulation solution to an activity concentration of 370-740 MBq/mL.

The following examples (for structures, see Table 9.1) were synthesized following the standard method:

Example H1 was synthesized from Example B2.

Example H2 was synthesized from Example B1.

Example H3 was synthesized from Example B3.

Example H4 was synthesized from Example B4.

TABLE 9.1
Examples of [¹⁷⁷Lu]Lu-labeled peptides synthesized following the general ¹⁷⁷Lu-radiolabeling methods

| Ex. No. | Structure/Example Name* | HPLC |
|---|---|---|
| H1 (SEQ ID NO: 94) | 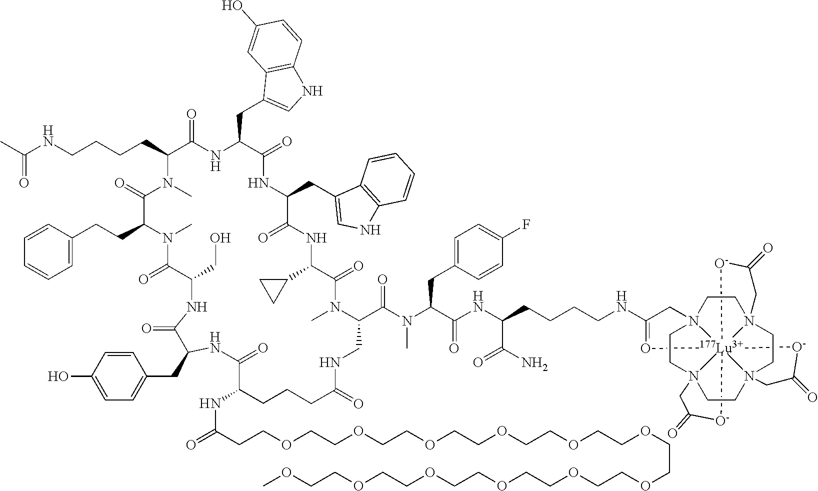 Example B2 labelled with ¹⁷⁷Lu | t_{R} = 7.58 min |
| H2 (SEQ ID NO: 95) | 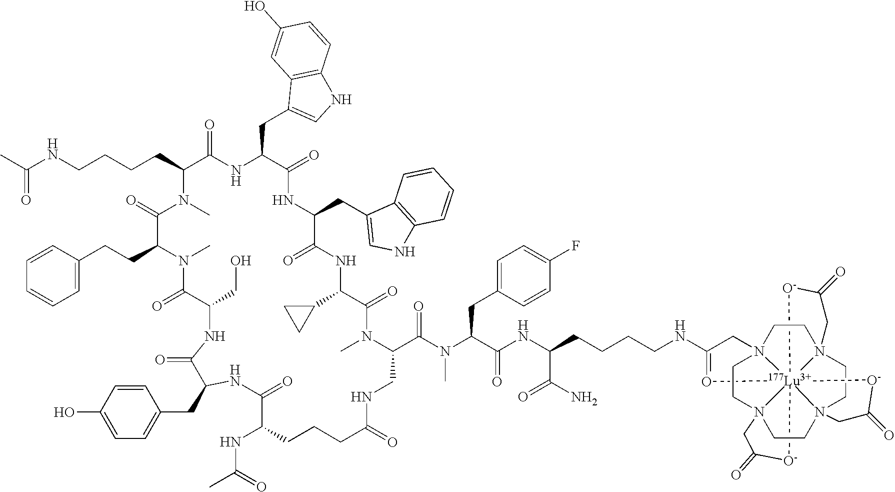 Example B1 labelled with ¹⁷⁷Lu | t_{R} = 7.47 min |
| H3 (SEQ ID NO: 96) | 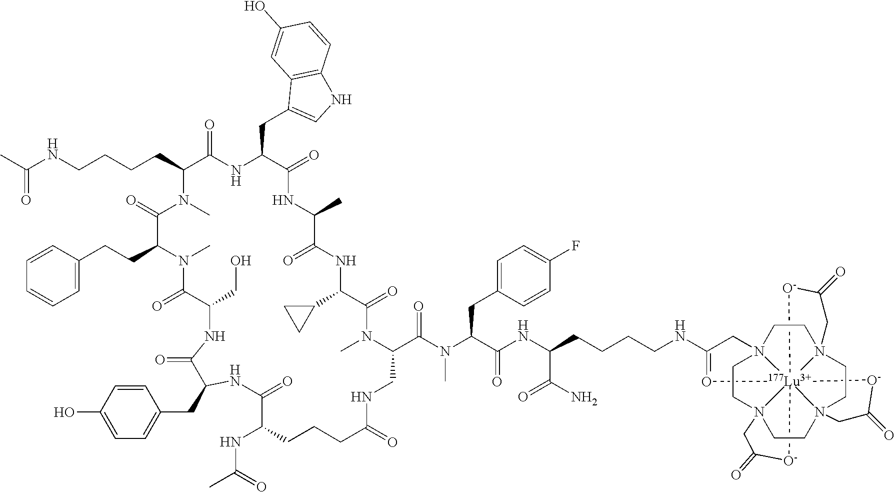 Example B3 labelled with ¹⁷⁷Lu | t_{R} = 7.03 min |
| H4 (SEQ ID NO: 97) | 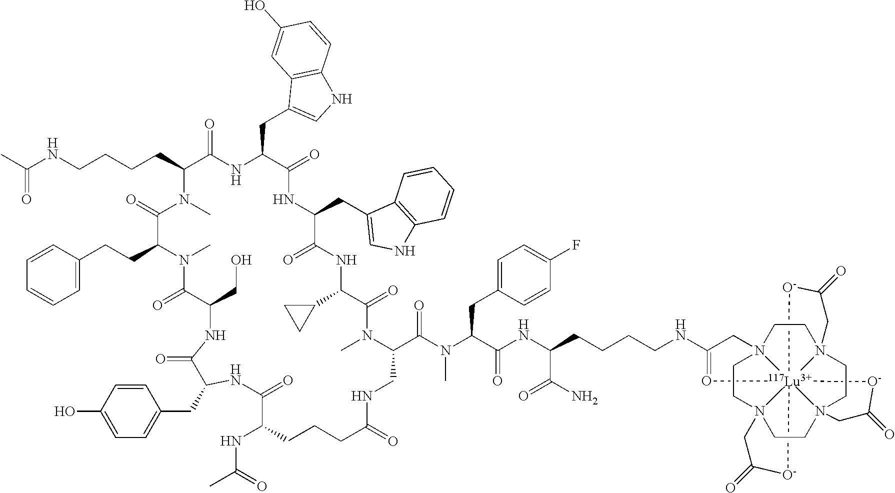 Example B4 labelled with ¹⁷⁷Lu | t_{R} = 6.85 min |

* The structures represent one way in which the [¹⁷⁷Lu]Lu-DOTA complexes (¹⁷⁷Lu radiolabeled - DOTA conjugates) could be illustrated. The same compound could be drawn, e.g., with a different number of coordination bonds, represented by either dashed or solid lines.

The initial radiochemical purity of the [¹⁷⁷Lu]Lu-labeled compounds was analyzed by radio-iTLC and radio-iTLC and the stability in solution at 24° C. was evaluated after the end of synthesis by radio-HPLC.

TABLE 9.2
Radiochemistry results for [¹⁷⁷Lu]Lu-labeled peptides

| | | Molar activity | | | | | | |
|---|---|---|---|---|---|---|---|---|
| Ex. | Radiolabeling | final product | Initial | Initial | 5 h | 24 h | 48 h | 72 h |
| No. | method | (MBq/nmol) | (iTLC) | (HPLC) | (%) | (%) | (%) | (%) |
| H1 | Method 1 | 37 | 99.6% | 99.4% | 98.5 | 98.2 | 98.0 | 97.4 |
| H1 | Method 1 | 74 | 97.5% | 98.3% | 97.3 | 95.4 | 93.5 | 92.5 |
| H2 | Method 1 | 37 | 98.4% | 97.5% | 97.5 | 97.1 | 96.6 | 96.1 |
| H2 | Method 1 | 74 | 99.6% | 97.7% | 97.9 | 97.0 | 94.5 | — |
| H2 | Method 2 | 74 | 98.6% | 99.8% | 95.9 | 93.6 | 93.6 | 93.2 |
| H3 | Method 2 | 37 | 99.8% | 98.5% | — | — | — | — |
| H4 | Method 2 | 74 | 99.6% | 99.6% | 98.6 | 98.2 | 97.6 | 97.8 |

9.4 Radiolabeling with [⁶⁸Ga]GaCl₃ (“I” Examples)

### Materials:

| Name | Supplier |
|---|---|
| Ethanol absolute ≥99.9% for analysis EMSURE ® | Merck |
| ACS, ISO, Reag. Ph Eur | |
| Hydrochloric acid 30% for inorganic trace analysis | Merck |
| [⁶⁸Ga]GaCl₃ in 0.1M hydrochloric acid solution | IRE Elit |
| | ⁶⁸Ge/⁶⁸Ga |
| | generator |
| Sodium acetate trihydrate for analysis EMSURE ® | Merck |
| ACS, ISO, Reag. Ph Eur | |
| Ascorbic acid ≥99.9998% (metals basis), TraceSELECT ™, | Honeywell |
| Fluka ™ | |
| Water TraceSELECT ™, for trace analysis | Honeywell |
| Phosphate Buffered Saline w/o Calcium w/o Magnesium w/o | VWR |
| Potassium Chloride | |
| Sodium hydroxide 2 mol/L (2N) in aqueous solution, Reagent | VWR |
| Grade | |
| Kolliphor ® HS 15 | Merck |

Labeling buffer: The labeling buffer consists of ascorbic acid (9.20 mg/mL) and sodium acetate (204 mg/mL) in TraceSELECT™ water. The labeling buffer was stored at 4-8° C.

Formulation solution: The formulation solution was prepared by mixing ascorbic acid (20 mg/ml), Kolliphor HS15 (0.3 mg/ml), and NaOH 2M (50 μl/ml) in PBS with 5% ethanol. The formulation solution was stored at 4-8° C.

Precursor stock solution for radiolabeling (1 mM): The DOTA precursors were dissolved in 1.0 mL ethanol. The solution was fractionated and stored in a freezer at −20° C.

Radiolabeling (10 MBq/nmol): In a glass vial containing 213.2 μL of the labeling buffer and 30 nmol of the DOTA-precursor stock solution (1 mM), it was added approximately 1.1 mL of [⁶⁸Ga]GaCl₃ in 0.1 M HCl (370 MBq) directly transferred from the ⁶⁸Ge/⁶⁸Ga generator. This reaction mixture, with pH 3.7, was left to stir (650 rpm) at 97° C. for 15 min in a shielded Thermoshaker Incubator. After cooling at room temperature, the final pH was corrected to ≥4.0 with 150 μL of the formulation solution and the radiochemical purity was followed by radio-HPLC without further purification.

The following examples (for structures, see Table 9.3) were synthesized following the standard method above:

Example I1 was synthesized from Example B2.

Example I2 was synthesized from Example B1.

TABLE 9.3
Examples of [⁶⁸Ga]Ga-labeled peptides synthesized following the general
⁶⁸Ga-labeling methods

| Ex. No. | Structure/Example Name* | HPLC |
|---|---|---|
| I1 (SEQ ID NO: 98) | 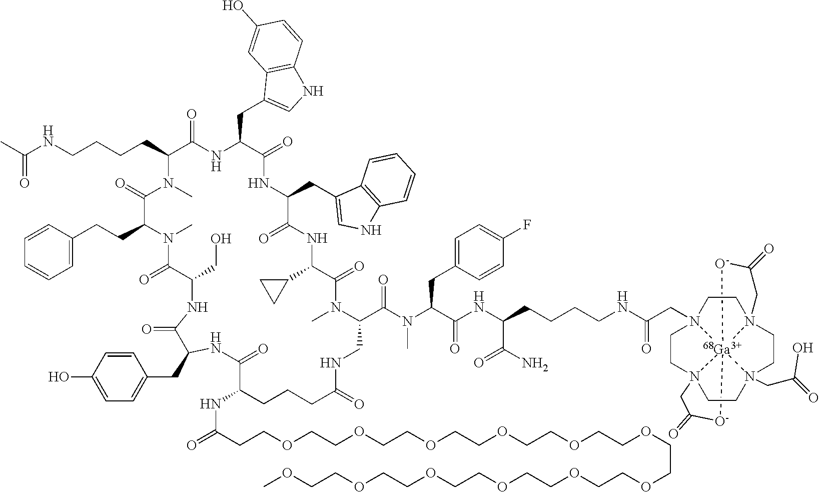 Example B2 labelled with ⁶⁸Ga | t_{R} = 7.60 min |
| I2 (SEQ ID NO: 99) | 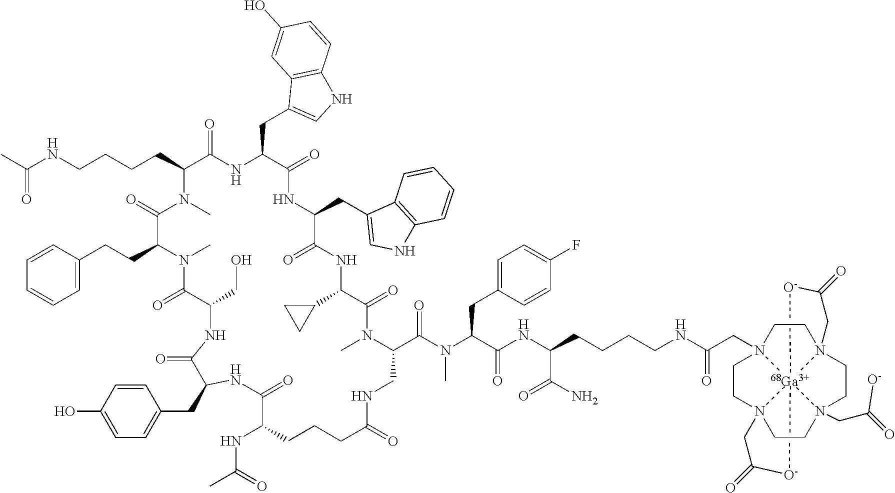 Example B1 labelled with ⁶⁸Ga | t_{R} = 7.50 min |

* The structures represent one way in which the [⁶⁸Ga]Ga-DOTA complexes (⁶⁸Ga radiolabeled- DOTA conjugates) could be illustrated. The same compound could be drawn, e.g., with a different number of coordination bonds, represented by either dashed or solid lines.

The initial radiochemical purity of [⁶⁸Ga]Ga-labeled compounds was analyzed by radio-HPLC and the stability in solution at 24° C. was evaluated 4 h after the end of synthesis by radio-HPLC.

TABLE 9.4
Radiochemistry results for [⁶⁸Ga]Ga-labeled peptides

| Example No. | Initial (HPLC) | 4 h |
|---|---|---|
| I1 | 99.1% | 97.2% |
| I2 | 97.7% | 97.2% |

9.5 Radiolabeling with [²²⁵Ac]AcCl₃ (“J” Examples)

### Materials

| Name | Supplier |
|---|---|
| Trometamol | Merck |
| Sodium ascorbate | ABX GmbH |
| Gentisic acid | Merck |
| Kolliphor HS15 | BASF |
| WFI | NA |
| Ac-225 | JSC, TerraPower, Panthera |
| CH₃COONH₄ | |
| CH₃OH | |

Radiolabeling buffer: Trometamol 0.25M at pH 8. The formulation solution was stored at room temperature.

Sodium ascorbate stock solution (100 mg/mL): 100 mg/mL sodium ascorbate. The formulation solution was stored at room temperature.

Gentisic acid stock solution (5 mg/mL): 5 mg/mL gentisic acid. The formulation solution was stored at room temperature.

Peptide stock solution for radiolabeling (1 mg/mL): The chemical precursor (Example B1) was dissolved with a 10 mg/mL Kolliphor HS15 solution. The formulation solution was stored at room temperature.

Radiolabeling (1 MBg/mL): 1.5 mL of Trometamol 0.25M buffer and 2 mL of chemical precursor (1000 μg/mL) were added to the reaction vial. Ac-225 was diluted in 0.04 M HCl to achieve an activity concentration of 50 MBq/mL. 1 mL of Ac-225 solution was added to the reaction vial, then 1.5 mL of WFI was used to rinse the source vial. The solutions were transferred to the reaction vial and gently mixed to homogenize the solution. The reaction mixture was heated at 95° C. for 20 min. After cooling to room temperature, the reaction mixture was transferred to a bulk glass vial. 5 mL of Sodium ascorbate stock solution (100 mg/mL), 10 mL of Gentisic acid stock solution (5 mg/mL) and 29 mL of 0.9% saline solution were added to the bulk vial to achieve a target volumetric activity of 1 MBq/mL and the solution was gently mixed until homogeneity.

The completion of the reaction was confirmed by TLC, and there was no need for further purification steps. RCP>97% was determined by TLC and stability up to 5 days was confirmed by TLC with RCP>95%.

TABLE 9.5
Example(s) of [²²⁵Ac]Ac-labeled peptides synthesized according to the ²²⁵Ac-
labeling methods

| Ex. No. | Structure/Example Name* |
|---|---|
| J1 (SEQ ID NO: 100) | 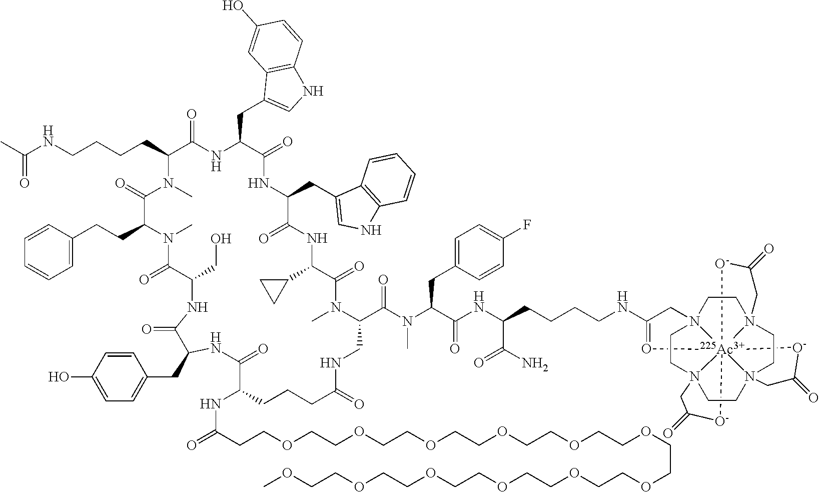 Example B2 labelled with ²²⁵Ac; J1 can be prepared in a manner analogous to the preparation of J2. |
| J2 (SEQ ID NO: 101) | 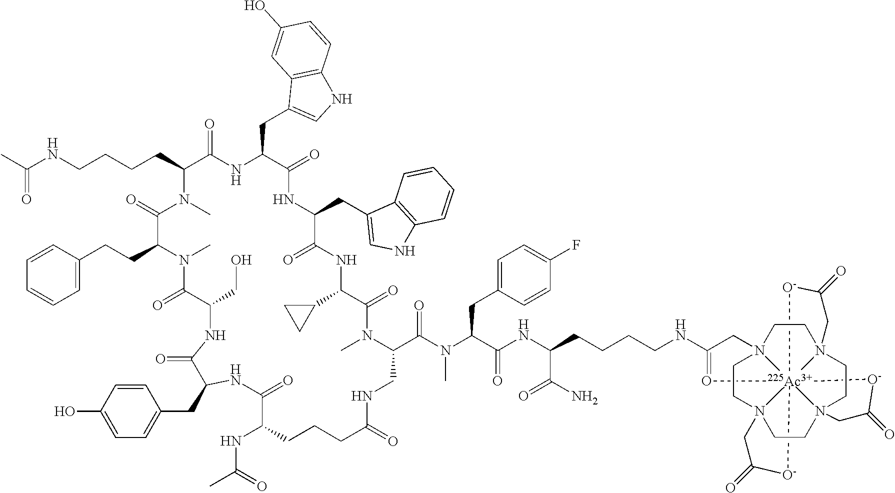 Example B1 labelled with ²²⁵Ac |

10 Building blocks

### 10.1 Resins

TABLE 10.1
Examples of Resins:

| No | Name | Details | Reference |
|---|---|---|---|
| R1 | Fmoc-Rink | Mesh size: 100-200 mesh, Loading: | Commercially available |
| | amide ProTide | 0.40-0.80 mmol/g | |
| | resin | | |
| R2 | Fmoc-Rink | Mesh size: 100-200 mesh, Loading: | Commercially available |
| | amide AM resin | 0.30-0.80 mmol/g | (CAS 183599-10-2) |
| R3 | Fmoc-Sieber | Mesh size: 100-200 mesh, Loading: | Commercially available |
| | amide resin | 0.48-0.65 mmol/g | (CAS 915706-90-0) |
| R4 | Fmoc- | Mesh size: 100-200 mesh, Loading: | Commercially available |
| | Lys(Boc)-Wang | 0.61 mmol/g | (CAS 65307-53-1) |
| | resin | | |

### 10.2 Fmoc-Amino Acid Building Blocks

TABLE 10.2
Examples of Fmoc-protected amino acid building blocks:

| | | | Reference or |
|---|---|---|---|
| Abbreviation | Structure | Name | LC-MS |
| Fmoc-L- Tyr(tBu)-OH | 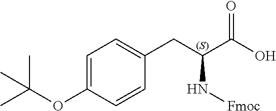 | (S)-2-((((9H-fluoren-9- yl)methoxy)carbonyl) amino)-3-(4-(tert- butoxy)phenyl)propanoic acid | Commercially available (CAS 71989-38-3) |
| Fmoc-D- Tyr(tBu)-OH | 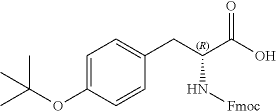 | (R)-2-((((9H-fluoren-9- yl)methoxy)carbonyl) amino)-3-(4-(tert- butoxy)phenyl)propanoic acid | Commercially available (CAS 118488-18-9) |
| Fmoc-L- 3PyA(6OH)- OH | 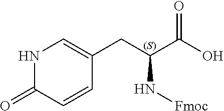 | (S)-2-((((9H-fluoren-9- yl)methoxy)carbonyl) amino)-3-(6-oxo-1,6- dihydropyridin-3- yl)propanoic acid | Commercially available (CAS 169555-94-6) |
| Fmoc-L- Glu(OtBu)-OH | 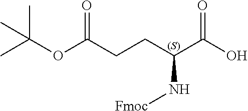 | (S)-2-((((9H-fluoren-9- yl)methoxy)carbonyl) amino)-5- (tert-butoxy)-5- oxopentanoic acid | Commercially available (CAS 71989-18-9) |
| Fmoc-D- Glu(OtBu)-OH | 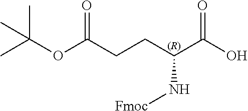 | (R)-2-((((9H-fluoren-9- yl)methoxy)carbonyl) amino)-5- (tert-butoxy)-5- oxopentanoic acid | Commercially available (CAS 104091-08-9) |
| Fmoc-L- Glu(OAll)-OH | 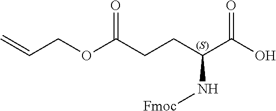 | (S)-2-((((9H-fluoren-9- yl)methoxy)carbonyl) amino)-5-(allyloxy)-5- oxopentanoic acid | Commercially available (CAS 133464-46-7) |
| Fmoc-L- Ser(tBu)-OH | 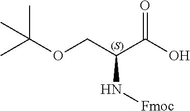 | N-(((9H-fluoren-9- yl)methoxy)carbonyl)-O- (tert-butyl)-L-serine | Commercially available (CAS 71989-33-8) |
| Fmoc-D- Ser(tBu)-OH | 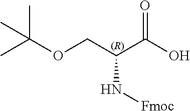 | N-(((9H-fluoren-9- yl)methoxy)carbonyl)-O- (tert-butyl)-D-serine | Commercially available (CAS 128107-47-1) |
| Fmoc-L- Ser(Trt)-OH | 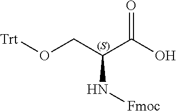 | N-(((9H-fluoren-9- yl)methoxy)carbonyl)-O- trityl-L-serine | Commercially available (CAS 111061-56-4) |
| Fmoc-L-N- Me-Ser(tBu)- OH | 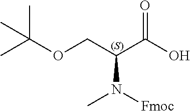 | N-(((9H-fluoren-9- yl)methoxy)carbonyl)-O- (tert-butyl)-N-methyl-L- serine | Commercially available (CAS 197632-77-2) |
| Fmoc-L-N- Me-Homo- Phe-OH | 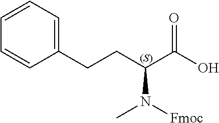 | (S)-2-((((9H-fluoren-9- yl)methoxy)carbonyl) (methyl)amino)-4- phenylbutanoic acid | Commercially available (CAS 1065076-30-3) |
| Fmoc-L-N- Me-Lys-OH | 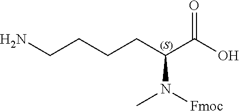 | N²-(((9H-fluoren-9- yl)methoxy)carbonyl)-N²- methyl-L-lysine | Commercially available (1436392-70-9) |
| Fmoc-L-N- Me-Lys(Ac)- OH | 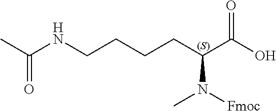 | N²-(((9H-fluoren-9- yl)methoxy)carbonyl)-N⁶- acetyl-N²-methyl-L-lysine | PeptiDream Inc. - US2023/257343, 2023, A1 |
| Fmoc-L-Trp(5- OH)-OH | 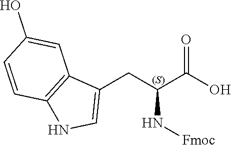 | (S)-2-((((9H-fluoren-9- yl)methoxy)carbonyl) amino)-3-(5- hydroxy-1H-indol- 3-yl)propanoic acid | Commercially available (CAS 178119-94-3) |
| Fmoc-L- Trp(Boc)-OH | 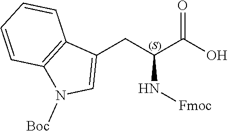 | N^{α}-(((9H-fluoren-9- yl)methoxy)carbonyl)-1- (tert-butoxycarbonyl)-L- tryptophan | Commercially available (CAS 143824-78-6) |
| Fmoc-D- Trp(Boc)-OH | 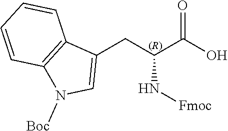 | N^{α}-(((9H-fluoren-9- yl)methoxy)carbonyl)-1- (tert-butoxycarbonyl)-D- tryptophan | Commercially available (CAS 163619-04-3) |
| Fmoc-L- Trp(8N)-OH | 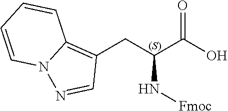 | (S)-2-((((9H-fluoren-9- yl)methoxy)carbonyl) amino)-3-(pyrazolo[1,5- a]pyridin-3-yl)propanoic acid | Commercially available (CAS 2350078-63-4) |
| Fmoc-L- CyclopropylGly- OH | 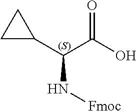 | (S)-2-((((9H-fluoren-9- yl)methoxy)carbonyl) amino)-2- cyclopropylacetic acid | Commercially available (CAS 1212257-18-5) |
| Fmoc-L-N- Me- Dap(Alloc)- OH | 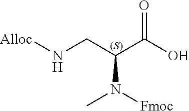 | (S)-2-((((9H-fluoren-9- yl)methoxy)carbonyl) (methyl)amino)-3- (((allyloxy)carbonyl) amino)propanoic acid | PeptiDream Inc. - US2023/257343, 2023, A1 |
| Fmoc-L-N- Me-Dap(Boc)- OH | 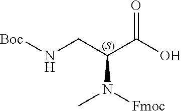 | (S)-2-((((9H-fluoren-9- yl)methoxy)carbonyl) (methyl)amino)-3-((tert- butoxycarbonyl)amino) propanoic acid | PeptiDream Inc. - US2023/257343, 2023, A1 |
| Fmoc-L- Dap(Boc)-OH | 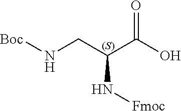 | (S)-2-((((9H-fluoren-9- yl)methoxy)carbonyl) amino)-3-((tert- butoxycarbonyl)amino) propanoic acid | Commercially available (CAS 162558-25-0) |
| Fmoc-L- Dap(Alloc)- OH | 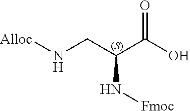 | (S)-2-((((9H-fluoren-9- yl)methoxy)carbonyl) amino)-3- (((allyloxy)carbonyl) amino)propanoic acid | Commercially available (CAS 188970-92-5) |
| Fmoc-L-N- Me- Dab(Alloc)- OH | 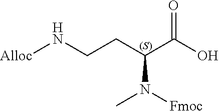 | (S)-2-((((9H-fluoren-9- yl)methoxy)carbonyl) (methyl)amino)-4- (((allyloxy)carbonyl)amin o)butanoic acid | PeptiDream Inc. - US2023/257343, 2023, A1 |
| Fmoc-L-N- Me-Dab(Boc)- OH | 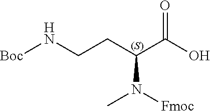 | (S)-2-((((9H-fluoren-9- yl)methoxy)carbonyl) (methyl)amino)-4-((tert- butoxycarbonyl)amino)but anoic acid | Commercially available (CAS 2044702-38-5) |
| Fmoc-L-N- Me-Phe(4F)- OH | 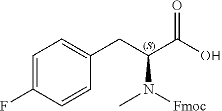 | (S)-2-((((9H-fluoren-9- yl)methoxy)carbonyl) (methyl)amino)-3-(4- fluorophenyl)propanoic acid | Commercially available (CAS 1979176-87-8) |
| Fmoc-L- Lys(Boc)-OH | 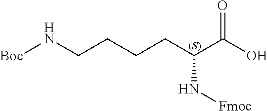 | N²-(((9H-fluoren-9- yl)methoxy)carbonyl)-N⁶- (tert-butoxycarbonyl)-L- lysine | Commercially available (CAS 71989-26-9) |
| Fmoc-D- Lys(Boc)-OH | 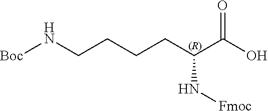 | N²-(((9H-fluoren-9- yl)methoxy)carbonyl)-N⁶- (tert-butoxycarbonyl)-D- lysine | Commercially available (CAS 92122-45-7) |
| Fmoc-L- Homo- Lys(Boc)-OH | 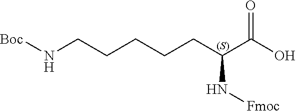 | (S)-2-((((9H-fluoren-9- yl)methoxy)carbonyl) amino)-7-((tert- butoxycarbonyl)amino) heptanoic acid | Commercially available (CAS 194718-17-7) |
| Fmoc-L- Lys(N3)-OH | 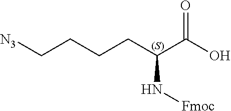 | N²-(((9H-fluoren-9- yl)methoxy)carbonyl)-N⁶- diazo-L-lysine | Commercially available (CAS 159610-89-6) |
| Fmoc-L-allo- Thr(tBu)-OH | 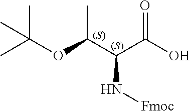 | N-(((9H-fluoren-9- yl)methoxy)carbonyl)-O- (tert-butyl)-L- allothreonine | Commercially available (CAS 201481-37-0) |
| BB1 | 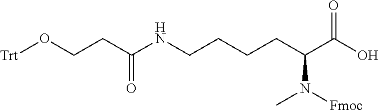 | N²-(((9H-fluoren-9- yl)methoxy)carbonyl)-N²- methyl-N⁶-(3- (trityloxy)propanoyl)-L- lysine | Synthesized as described below, LCMS method AB-2 tR = 1.95 min; [M-Trt + H]⁺ = 455.3 |
| Fmoc-L-N- Me- K(COCH2O (tBu))-OH | 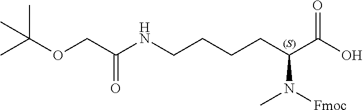 | N²-(((9H-fluoren-9- yl)methoxy)carbonyl)-N⁶- (2-(tert-butoxy)acetyl)-N²- methyl-L-lysine | PeptiDream Inc. - US2023/257343, 2023, A1 |
| Fmoc-L- K(COpipzaa) (OtBu)-OH | 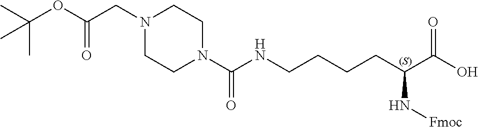 | N²-(((9H-fluoren-9- yl)methoxy)carbonyl)-N⁶- (4-(2-(tert-butoxy)-2- oxoethyl)piperazine-1- carbonyl)-L-lysine | US2023/257343, 2023, A1 |
| Fmoc-L-Beta- Ala-OH | 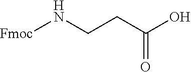 | 3-((((9H-fluoren-9- yl)methoxy)carbonyl) amino)propanoic acid | Commercially available (CAS 35737-10-1) |
| Fmoc-L- Homo- Glu(OAll)-OH | 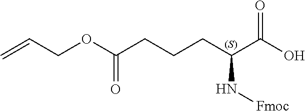 | (S)-2-((((9H-fluoren-9- yl)methoxy)carbonyl) amino)-6-(allyloxy)-6- oxohexanoic acid | Commercially available (CAS 133464-45-6) |
| Fmoc-L- Homo- Glu(OtBu)-OH | 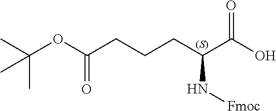 | (S)-2-((((9H-fluoren-9- yl)methoxy)carbonyl) amino)-6-(tert-butoxy)-6- oxohexanoic acid | Commercially available (CAS 159751-47-0) |
| Fmoc-L-N- Me- Glu(OtBu)-OH | 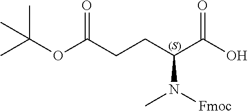 | (S)-2-((((9H-fluoren-9- yl)methoxy)carbonyl) (methyl)amino)-5-(tert- butoxy)-5-oxopentanoic acid | Commercially available (CAS 200616-40-6) |
| Fmoc-L-Ala- OH | 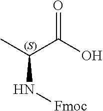 | (((9H-fluoren-9- yl)methoxy)carbonyl)-L- alanine | Commercially available (CAS 35661-39-3) |
| Fmoc-L-Gly- OH | 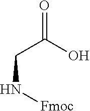 | (((9H-fluoren-9- yl)methoxy)carbonyl) glycine | Commercially available (CAS 29022-11-5) |
| Fmoc-L- Asp(OtBu)- OH | 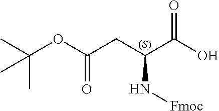 | (S)-2-((((9H-fluoren-9- yl)methoxy)carbonyl) amino)-4-(tert-butoxy)-4- oxobutanoic acid | Commercially available (CAS 71989-14-5) |
| Fmoc-L-N- Me- Asp(OtBu)- OH | 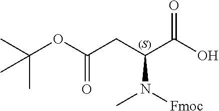 | (S)-2-((((9H-fluoren-9- yl)methoxy)carbonyl) (methyl)amino)-4-(tert- butoxy)-4-oxobutanoic acid | Commercially available (CAS 152548-66-8) |
| Fmoc-L-Phe- OH | 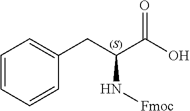 | (((9H-fluoren-9- yl)methoxy)carbonyl)-L- phenylalanine | Commercially available (CAS 35661-40-6) |
| Fmoc-D-Phe- OH | 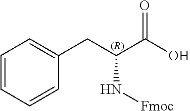 | (((9H-fluoren-9- yl)methoxy)carbonyl)-D- phenylalanine | Commercially available (CAS 86123-10-6) |
| Fmoc-L- Cys(Trt)-OH | 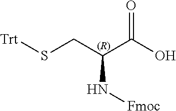 | N-(((9H-fluoren-9- yl)methoxy)carbonyl)-S- trityl-L-cysteine | Commercially available (CAS 103213-32-7) |
| Fmoc-L-N- Me-Cys(Trt)- OH | 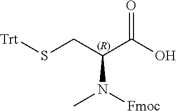 | N-(((9H-fluoren-9- yl)methoxy)carbonyl)-N- methyl-S-trityl-L-cysteine | Commercially available (CAS 944797-51-7) |
| Fmoc-L- Homo- Cys(Trt)-OH | 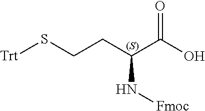 | N-(((9H-fluoren-9- yl)methoxy)carbonyl)-S- trityl-L-homocysteine | Commercially available (CAS 167015-23-8) |
| Fmoc-L-N- Me-Homo- Cys(Trt)-OH | 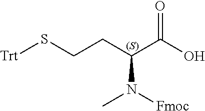 | N-(((9H-fluoren-9- yl)methoxy)carbonyl)-N- methyl-S-trityl-L- homocysteine | Commercially available (CAS 526210-71-9) |
| Fmoc-L-Pro- OH | 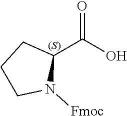 | (((9H-fluoren-9- yl)methoxy)carbonyl)-L- proline | Commercially available (CAS 71989-31-6) |
| Fmoc-L- Orn(Boc)-OH | 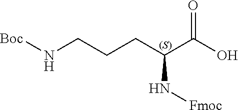 | (S)-2-((((9H-fluoren-9- yl)methoxy)carbonyl) amino)-5-((tert- butoxycarbonyl)amino) pentanoic acid | Commercially available (CAS 109425-55-0) |
| Fmoc-L- Trp(5OMe)- OH | 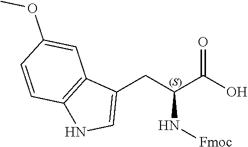 | (S)-2-((((9H-fluoren-9- yl)methoxy)carbonyl) amino)-3-(5-methoxy-1H- indol-3-yl)propanoic acid | Commercially available (CAS 460751-69-3) |
| Fmoc-Ahp-OH | 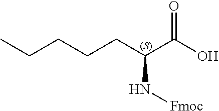 | (S)-2-((((9H-fluoren-9- yl)methoxy)carbonyl) amino)heptanoic acid | Commercially available (CAS 1197020-22-6) |
| Fmoc-L-N- Me-Ahp-OH | 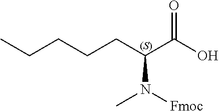 | (S)-2-((((9H-fluoren-9- yl)methoxy)carbonyl) (methyl)amino)heptanoic acid | Commercially available (CAS 2642726-02-9) |
| Fmoc-L-Aoc- OH | 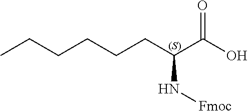 | (S)-2-((((9H-fluoren-9- yl)methoxy)carbonyl) amino)octanoic acid | Commercially available (CAS 888725-91-5) |
| Fmoc-L-N- Me-Aoc-OH | 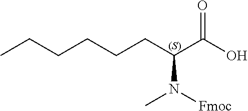 | (S)-2-((((9H-fluoren-9- yl)methoxy)carbonyl) (methyl)amino)octanoic acid | Commercially available (CAS 2389078-28-6) |
| BB2 | 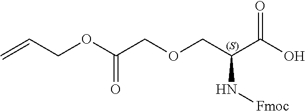 | N-(((9H-fluoren-9- yl)methoxy)carbonyl)-O- (2-(allyloxy)-2-oxoethyl)- L-serine | J. Org. Chem. 1992 57 (24), 6421-6430 (CAS 133486- 74-5) |
| Fmoc-L-N- Me-Homo- Phe(3Cl)-OH | 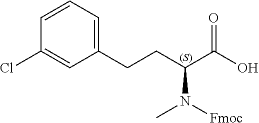 | (S)-2-((((9H-fluoren-9- yl)methoxy)carbonyl) (methyl)amino)-4-(3- chlorophenyl)butanoic acid | Commercially available (CAS 2255321-19-6) |
| Fmoc-L- bA(2S-Me)- OH | 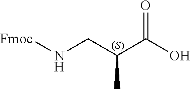 | (R)-3-((((9H-fluoren-9- yl)methoxy)carbonyl) amino)-2- methylpropanoic acid | Commercially available (CAS 203854-58-4) |
| Fmoc-L- bA(2S-OH)- OH | 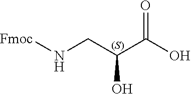 | (S)-3-((((9H-fluoren-9- yl)methoxy)carbonyl) amino)-2- hydroxypropanoic acid | Commercially available (CAS 172721-23-2) |
| Fmoc-beta-2- Glu(OtBu)-OH | 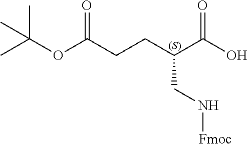 | (S)-2-(((((9H-fluoren-9- yl)methoxy)carbonyl) amino)methyl)-5- (tert-butoxy)- 5-oxopentanoic acid | Commercially available (CAS 941292-99-5) |
| BB3 | 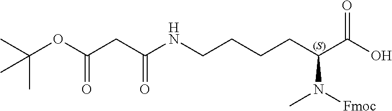 | N²-(((9H-fluoren-9- yl)methoxy)carbonyl)-N⁶- (3-(tert-butoxy)-3- oxopropanoyl)-N²-methyl- L-lysine | Synthesized as described below, LCMS method AB-1 t_{R} = 1.13 min; [M + H]⁺ = 547.3 |
| BB4 | 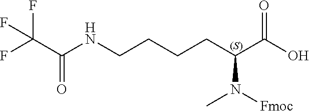 | N²-(((9H-fluoren-9- yl)methoxy)carbonyl)-N²- methyl-N⁶-(2,2,2- trifluoroacetyl)-L-lysine | Synthesized as described below, LCMS method AB-1 t_{R} = 1.09 min; [M + H]⁺ = 479.3 |
| Fmoc-L- F3AA(OtBu)- OH | 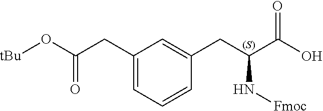 | (S)-2-((((9H-fluoren-9- yl)methoxy)carbonyl) amino)-3-(3-(2-(tert- butoxy)-2- oxoethyl)phenyl)propanoic acid | PeptiDream Inc. - US2023/257343, 2023, A1 |
| Fmoc-L-Alk1- OH | 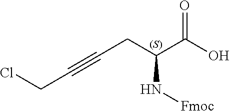 | (S)-2-((((9H-fluoren-9- yl)methoxy)carbonyl) amino)-6-chlorohex- 4-ynoic acid | Commercially available (CAS 2382461-67-6) |
| Fmoc- 3(CH₂NH₂)Bz | 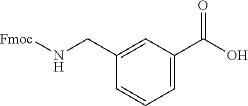 | 3-(((((9H-fluoren-9- yl)methoxy)carbonyl) amino)methyl) benzoic acid | Commercially available (CAS 155369-11-2) |
| Fmoc-PEG10- OH | 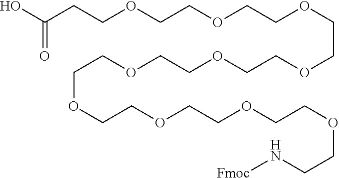 | 1-(9H-fluoren-9-yl)-3-oxo- 2,7,10,13,16,19,22,25,28,3 1,34-undecaoxa-4- azaheptatriacontan-37-oic acid | Commercially available (CAS 2101563-45-3) |
| BB5 | 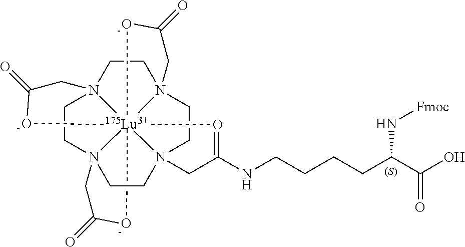 | (S)-2,2′,2″-(10-(2-((5- ((((9H-fluoren-9- yl)methoxy)carbonyl) amino)-5- carboxypentyl)amino)- (((¹⁷⁵lutetium(3+)))-2- oxoethyl)-1,4,7,10- tetraazacyclododecane- 1,4,7-triyl)triacetic acid) | Synthesized as described below, LCMS method AB-3 t_{R} = 2.817 min; [M + H]⁺ = 927.2 |
| Fmoc- propargyl-Gly- OH | 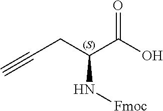 | (S)-2-((((9H-fluoren-9- yl)methoxy)carbonyl) amino)pent-4- ynoic acid | Commercially available (CAS 198561-07-8) |
| Fmoc-Sar-OH | 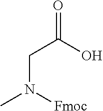 | N-(((9H-fluoren-9- yl)methoxy)carbonyl)-N- methylglycine | Commercially available (CAS 77128-70-2) |
| Fmoc-L- Lys(Alloc)-OH | 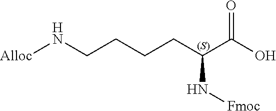 | N²-(((9H-fluoren-9- yl)methoxy)carbonyl)-N⁶- ((allyloxy)carbonyl)-L- lysine | Commercially available (CAS 146982-27-6) |
| Alloc-L- Lys(Fmoc)- OH | 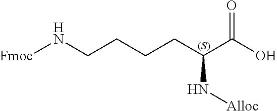 | N⁶-(((9H-fluoren-9- yl)methoxy)carbonyl)-N²- ((allyloxy)carbonyl)-L- lysine | Commercially available (CAS 186350-56-1) |

Certain amino acids were synthesized as described below.

### 10.2.1 Synthesis of N₂-(((9H-fluoren-9-yl) methoxy) carbonyl)-N₆-(3-(tert-butoxy)-3-oxopropanoyl)-N²-methyl-L-lysine (BB3)

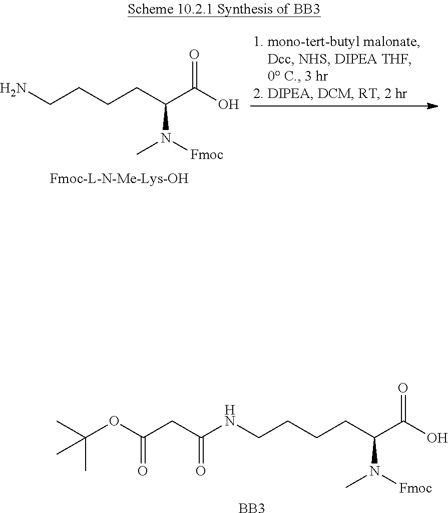

Step 1-1: To a solution of mono-tert-butyl malonate (CAS 40052-13-9, 1.996 g, 1.92 mL, 4 eq., 12.46 mmol) and NHS (1.434 g, 4 eq., 12.46 mmol) in THF (15 mL) at 0° C. was added a solution of DCC (2.571 g, 4 eq., 12.46 mmol) in THF (30 mL). The reaction mixture was stirred at 0° C. for 3 hr. The reaction mixture was filtered, and the filtrate was concentrated to dryness in vacuo to afford the crude intermediate.

Step 1-2: The crude intermediate from Step 1-1 was added to a solution of Fmoc-L-N-Me-Lys-OH (1.45 g, 90% Wt, 1 eq., 3.115 mmol, HCl salt) and DIPEA (1.208 g, 1.63 mL, 3 eq., 9.345 mmol) in DCM (20 mL) at RT. The reaction mixture was stirred at RT for 2 hr. The reaction mixture was filtered, and the filtrate was concentrated to dryness in vacuo. The resulting residue was purified by silica gel flash column chromatography (gradient, DCM/MeOH=100/0 to 96/4) to give compound BB3 (1.188 g, 2.20 mmol, 71% yield) as a white solid. 1H NMR (400 MHZ, DMSO-d₆) δ 12.76 (s, 1H), 7.96 (t, J=5.6 Hz, 1H), 7.89 (t, J=6.6 Hz, 2H), 7.68-7.58 (m, 2H), 7.41 (td, J=7.3, 3.9 Hz, 2H), 7.32 (tdd, J=11.3, 5.3, 2.9 Hz, 2H), 4.49 (dd, J=11.0, 4.7 Hz, 1H), 4.41-4.32 (m, 2H), 4.32-4.23 (m, 1H), 3.07 (d, J=2.6 Hz, 2H), 3.06-2.99 (m, 2H), 2.76-2.69 (m, 3H), 1.86-1.53 (m, 2H), 1.45-1.41 (m, 1H), 1.38 (d, J=1.7 Hz, 9H), 1.18 (ddt, J=20.9, 14.6, 8.3 Hz, 2H). LCMS method AB-1, t_{R}=1.13 min; [M+Na]⁺=547.3.

### 10.2.2 Synthesis of N²-(((9H-fluoren-9-yl) methoxy) carbonyl)-N²-methyl-N⁶-(2,2,2-trifluoroacetyl)-L-lysine (BB4)

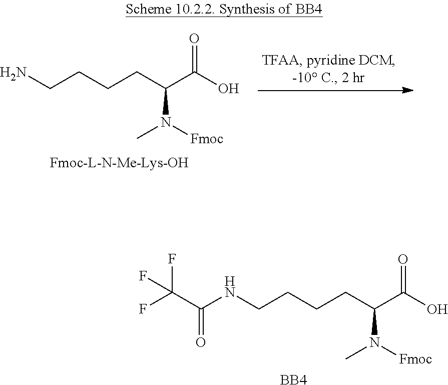

To a solution of Fmoc-L-N-Me-Lys-OH (500 mg, 90% Wt, 1 eq., 1.1 mmol) in DCM (8 mL) at −10° C. were added pyridine (420 mg, 0.43 mL, 5 eq., 5.4 mmol) and TFAA (230 mg, 0.15 mL, 1 eq., 1.1 mmol). The reaction mixture was stirred at −10° C. for 1.5 hr. Then another portion of pyridine (0.42 g, 0.43 mL, 5 eq., 5.4 mmol) and TFAA (0.23 g, 0.15 mL, 1 eq., 1.1 mmol) were added and the reaction mixture stirred at −10° C. for 0.5 hr. The reaction mixture was quenched with water at 0° C. and diluted with DCM. The mixture was washed two times with aq. HCl (0.2 M). The organic layer was dried over Na₂SO₄, filtered, and concentrated to dryness in vacuo. The residue was purified by silica gel flash column chromatography (gradient, DCM/MeOH=100/0 to 96/4) to give compound BB4 (310 mg, 0.62 mmol, 57% yield) as a white solid. 1H NMR (400 MHZ, DMSO-d₆) δ 12.79 (s, 1H), 9.41 (s, 2H), 7.89 (t, J=7.4 Hz, 4H), 7.68-7.58 (m, 4H), 7.41 (q, J=6.9 Hz, 4H), 7.32 (q, J=6.6 Hz, 4H), 4.48 (dd, J=11.0, 4.6 Hz, 1H), 4.42-4.22 (m, 7H), 3.17 (q, J=6.7 Hz, 4H), 2.71 (d, J=8.1 Hz, 6H), 1.47 (dq, J=23.0, 7.3 Hz, 4H), 1.17 (p, J=7.6 Hz, 3H), 1.11-1.05 (m, 1H). LCMS method AB-1, t_{R}=1.09 min; [M+H]⁺=479.3.

### 10.2.3 Synthesis of N²-(((9H-fluoren-9-yl) methoxy) carbonyl)-N²-methyl-N⁶-(3-(trityloxy) propanoyl)-L-lysine (BB1)

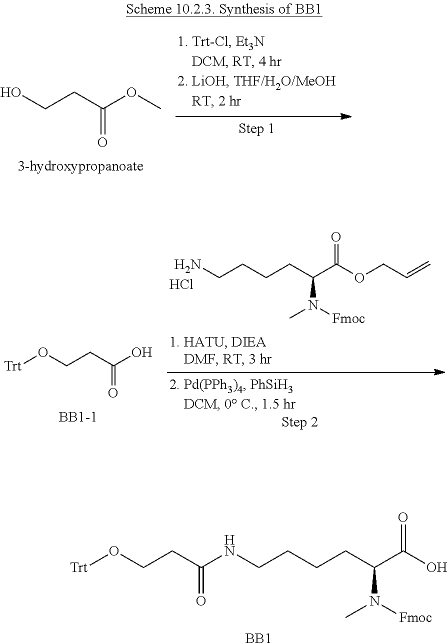

### Step 1:3-(trityloxy) propanoic acid (BB1-2)

Step 1-1: To a solution of methyl 3-hydroxypropanoate (CAS: 6149-41-3, 1.52 g, 1 eq., 12.7 mmol) in DCM (63.5 mL) at RT were added Et₃N (1.29 g, 1 eq., 12.7 mmol) and trityl chloride (CAS: 76-83-5, 3.54 g, 1 eq., 12.7 mmol). The mixture was stirred at RT for 4 hr and then diluted with H₂O. The mixture was extracted three times with EtOAc. The combined organic extracts were dried over Na₂SO₄, filtered, and concentrated in vacuo. The resulting residue was purified by silica gel flash column chromatography (gradient, hexane/EtOAc=100/0 to 50/50). Appropriate fractions were collected and concentrated in vacuo.

Step 1-2: To a solution of the resulting residue from Step 1.1 (2.58 g, 1 eq., 7.45 mmol) in THF/H₂O/MeOH (12 mL/12 mL/12 mL) at RT was added lithium hydroxide (CAS: 1310-65-2, 0.178 g, 1 eq., 7.45 mmol). The mixture was stirred at RT for 2 hr. The reaction mixture was washed with diisopropyl ether three times. The aqueous layer was acidified with 1M HCl to adjust to pH 5-6 and then extracted with EtOAc. The combined organic extracts were dried over Na₂SO₄, filtered, and concentrated in vacuo to afford BB1-2 (1.36 g, 3.81 mmol, 30% yield) as a white powder. 1H NMR (500 MHz, DMSO-d6) ¿ 12.27 (s, 1H), 7.40-7.16 (m, 15H), 3.18 (t, J=6.4 Hz, 2H), 2.49-2.47 (m, 2H).

### Step 2: Synthesis of N²-(((9H-fluoren-9-yl) methoxy) carbonyl)-N²-methyl-N⁶-(3-(trityloxy) propanoyl)-L-lysine (BB1)

Step 2-1: To a solution of prop-2-en-1-yl (2S)-6-amino-2-({[(9H-fluoren-9-yl) methoxy]carbonyl}(methyl)amino) hexanoate hydrochloride (CAS: 2973752-98-4, 2.00 g, 1.07 eq., 4.36 mmol) and BB1-2 (1.36 g, 1 eq., 4.09 mmol) in DMF (20.5 mL) at RT were added DIPEA (0.845 g, 1.17 mL, 1.5 eq., 6.54 mmol) and HATU (1.99 g, 1.2 eq., 5.23 mmol). The mixture was stirred at RT for 3 hr and then diluted with H₂O. The mixture was extracted three times with EtOAc. The combined organic extracts were washed with brine, dried over Na₂SO₄, filtered, and concentrated in vacuo. The resulting residue was purified by silica gel flash column chromatography (gradient, hexane/EtOAc=100/0 to 0/100). Appropriate fractions were collected and concentrated in vacuo.

Step 2-2: To a solution of the resulting residue from Step 2.1 (2.30 g, 3.12 mmol) in DCM (46 mL) at 0° C. were added phenylsilane (0.676 g, 0.770 ml, 2 eq., 6.24 mmol) and Pd(PPh₃)₄ (0.090 g, 0.025 eq., 0.078 mmol). The mixture was stirred at 0° C. for 1.5 hr, and then concentrated in vacuo. The resulting residue was purified by silica gel flash column chromatography (gradient, DCM/MeOH=100/0 to 70/30). Appropriate fractions were collected and concentrated in vacuo to afford the title compound BB1 (1.92 g, 2.59 mmol, 63% yield) as a brown powder. 1H NMR (500 MHZ, DMSO-d6) § 12.82 (s, 1H), 7.97 (t, J=5.3 Hz, 1H), 7.92-7.86 (m, 2H), 7.67-7.53 (m, 2H), 7.44-7.17 (m, 19H), 4.50-4.18 (m, 4H), 3.19-3.01 (m, 4H), 2.73-2.68 (m, 3H), 2.42-2.29 (m, 2H), 1.87-1.53 (m, 2H), 1.51-1.33 (m, 2H), 1.31-1.07 (m, 2H). LCMS method AB-2, t_{R}=1.95 min; [M-Trt+H]⁺=455.3.

### 10.2.4 Synthesis of(S)-2,2′,2″-(10-(2-((5-((((9H-fluoren-9-yl) methoxy) carbonyl)amino)-5-carboxypentyl)amino)-(((¹⁷⁵lutetium (3+)))-2-oxoethyl)-1,4,7,10-tetraazacyclododecane-1,4,7-triyl)triacetic acid) (BB5)

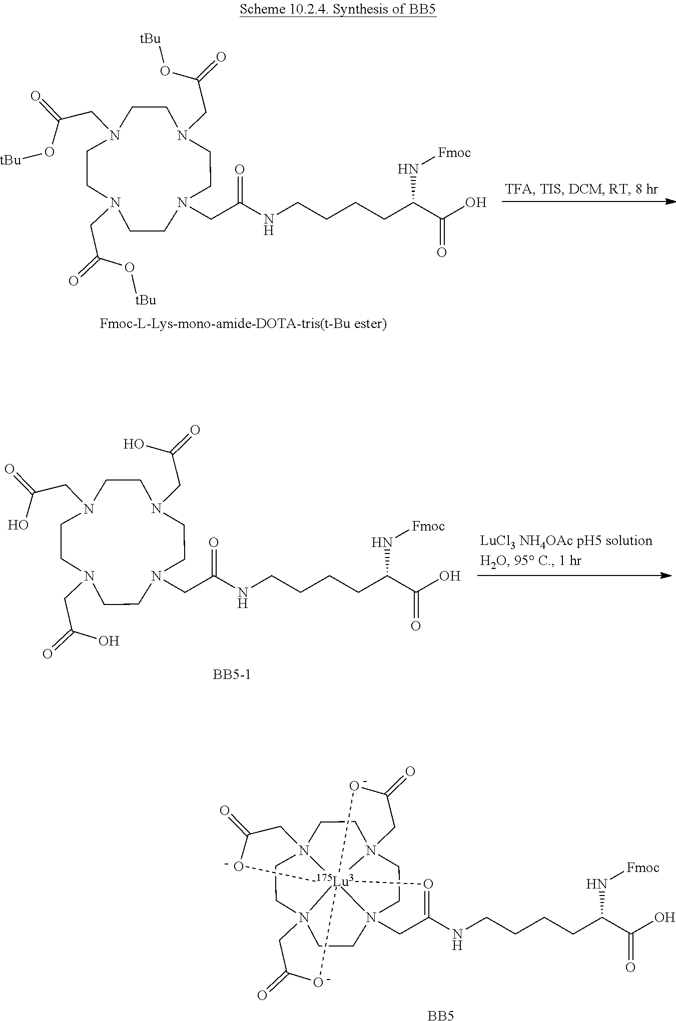

### Step 1: BB5-1

To a solution of Fmoc-L-Lys-mono-amide-DOTA-tris(t-Bu ester) (CAS: 479081-06-6, 52.4 g, 1 eq., 56.76 mol) and triisopropylsilane (36 mL, 27.9 g, 3.1 eq., 175.96 mmol) in DCM (1.05 L) was added TFA (437 mL, 647.2 g, 100 eq., 5.68 mol). The reaction mixture was stirred at RT for 15 hr. After concentration in vacuo, the residue was triturated with MTBE (4×200 mL). After the final trituration, the filter cake was re-dissolved in ACN (150 mL) and concentrated in vacuo to afford crude compound BB5-1 (53.80 g, 44.43 mmol, 78% yield) as a yellow solid. 1H NMR (400 MHZ, DMSO-d6) δ 8.50 (s, 1H), 7.91 (d, J=7.5 Hz, 2H), 7.74 (dd, J=7.2, 3.2 Hz, 2H), 7.64 (d, J=8.0 Hz, 1H), 7.43 (t, J=7.4 Hz, 2H), 7.34 (t, J=7.3 Hz, 2H), 4.37-4.18 (m, 3H), 4.11 (s, 2H), 3.93 (dd, J=13.6, 8.7 Hz, 3H), 3.79-3.48 (m, 4H), 3.33-2.59 (m, 12H), 2.08 (s, 4H), 1.72 (d, J=7.0 Hz, 1H), 1.66-1.53 (m, 1H), 1.53-1.24 (m, 4H).

### Step 2: BB5

To a clear solution of compound BB5-1 (167.6 g, 1 eq., 138.41 mmol) in aq. ammonium acetate solution (1 M) (1.25 L, 1.25 mol) and H₂O (1.34 L) was added lutetium chloride hexahydrate (64.70 g, 1.2 eq., 166.09 mmol). The reaction mixture was stirred at 95° C. for 1 hr. The reaction mixture was cooled to RT. After filtration, the filtrate was concentrated in vacuo to 200 mL and then purified by R^{P} Flash Chromatography (Stationary phase: C18 irregular 60 Å, 330 g; Eluent A: H₂O+0.1% Formic acid and eluent B: ACN, 5% to 100% Solvent B). After concentration, the desired product was obtained as a yellow solid (88 g, 94.96 mmol, 96% in HPLC, 68.0% yield over two steps). 1H NMR (400 MHZ, DMSO-d6) δ 9.59 (s, 1H), 7.90 (d, J=7.5 Hz, 2H), 7.75 (d, J=6.9 Hz, 2H), 7.65 (s, 1H), 7.43 (t, J=7.4 Hz, 2H), 7.34 (t, J=7.4 Hz, 2H), 4.26 (t, J=7.2 Hz, 3H), 3.94 (d, J=5.1 Hz, 1H), 3.72 (d, J=102.0 Hz, 8H), 3.18 (s, 8H), 2.83 (s, 3H), 2.67 (dd, J=11.5, 9.7 Hz, 4H), 2.33 (s, 4H), 1.76-1.55 (m, 2H), 1.49 (s, 2H), 1.42-1.28 (m, 2H). LCMS method AB-3, t_{R}=2.817 min; [M+H]⁺=927.2.

### 10.3 Other Building Blocks

TABLE 10.3
Examples of other building blocks:

| Abbreviation | Structure | Name | Reference |
|---|---|---|---|
| tert- butoxyacetic acid | 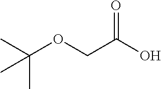 | 2-(tert-butoxy)acetic acid | Commercially available (CAS 13211-32-0) |
| Glutaric acid mono-tert- butyl ester | 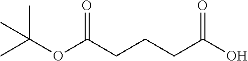 | 5-(tert-butoxy)-5- oxopentanoic acid | Commercially available (CAS 63128-51-8) |
| Chloroacetic acid | 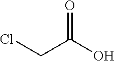 | 2-chloroacetic acid | Commercially available (CAS 79-11-8) |
| BB6 | 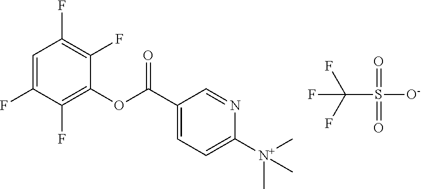 [SMILES: C[N+](C)(C)c1ccc(C(=O)Oc2c(F)c(F)cc(F)c2F)cn1 ; O=S(=O)([O-])C(F)(F)F] | N,N,N-trimethyl-5- ((2,3,5,6- tetrafluorophenoxy) carbonyl)pyridin-2- aminium trifluoromethanesulfonate | Commercially available (CAS 1246467-94-6) |
| m-PEG12- NHS ester | 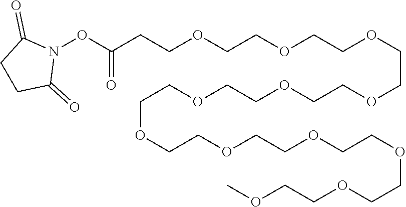 [SMILES: COCCOCCOCCOCCOCCOCCOCCOCCOCCOCCOCCOCCC(=O)ON1C(=O)CCC1=O] | 2,5-dioxopyrrolidin-1- yl 2,5,8,11,14,17,20,23,2 6,29,32,35- dodecaoxaoctatriacontan- 38-oate | Commercially available (CAS 174569-25-6) |
| DOTA-NHS ester | 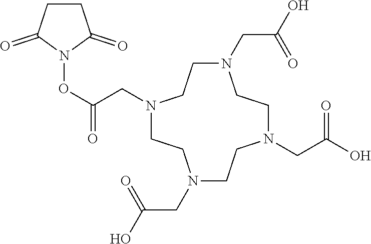 [SMILES: O=C(O)CN1CCN(CC(=O)O)CCN(CC(=O)ON2C(=O)CCC2=O)CCN(CC(=O)O)CC1] | 2,2′,2″-(10-(2-((2,5- dioxopyrrolidin-1- yl)oxy)-2-oxoethyl)- 1,4,7,10- tetraazacyclododecane- 1,4,7-triyl)triacetic acid | Commercially available (170908-81-3) |
| SulfoCy5- NHS ester | 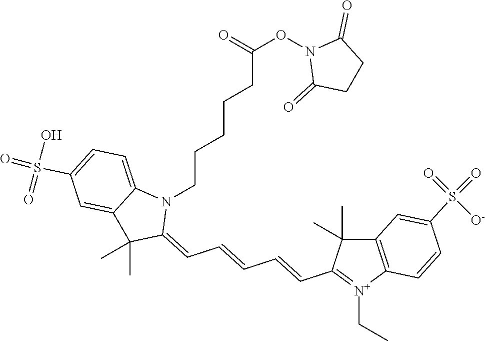 [SMILES: CC[N+]1=C(/C=C/C=C/C=C2\N(CCCCCC(=O)ON3C(=O)CCC3=O)c3ccc(S(=O)(=O)O)cc3C2(C)C)C(C)(C)c2cc(S(=O)(=O)[O-])ccc21] | 2-((1E,3E)-5-((Z)-1- (6-((2,5- dioxopyrrolidin-1- yl)oxy)-6-oxohexyl)- 3,3-dimethyl-5- sulfoindolin-2- ylidene)penta-1,3- dien-1-yl)-1-ethyl-3,3- dimethyl-3H-indol-1- ium-5-sulfonate | Commercially available (CAS 146368-14-1) |
| BB7 | 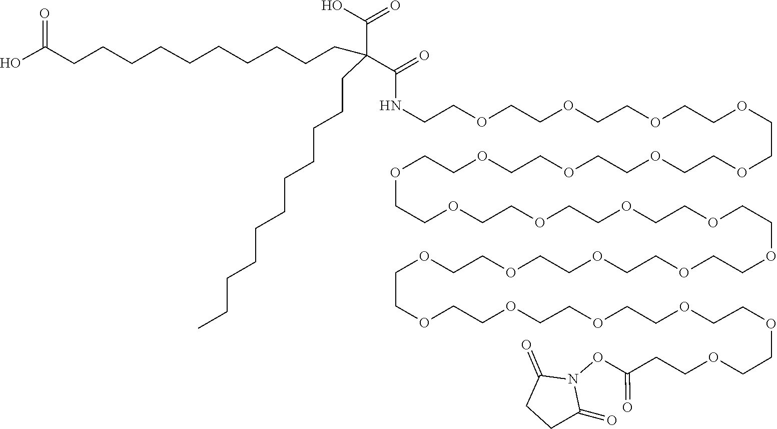 [SMILES: CCCCCCCCCCCC(CCCCCCCCCCC(=O)O)(C(=O)O)C(=O)NCCOCCOCCOCCOCCOCCOCCOCCOCCOCCOCCOCCOCCOCCOCCOCCOCCOCCOCCOCCOCCOCCOCCOCCOCCC(=O)ON1C(=O)CCC1=O] | 2-((75-((2,5- dioxopyrrolidin-1- yl)oxy)-75-oxo- 3,6,9,12,15,18,21,24,2 7,30,33,36,39,42,45,4 8,51,54,57,60,63,66,6 9,72- tetracosaoxapentaheptacontyl) carbamoyl)-2- undecyltridecanedioic acid | Novartis AG, U.S. 9,266,925, 2016, B2, incorporated herein by reference in its entirety |
| 1H-Tetrazole- 5-acetic acid | 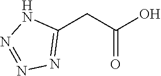 | 2-(1H-tetrazol-5- yl)acetic acid | Commercially available CAS: 21743-75-9 |
| Boc-L-Beta- Ala-OH | 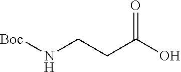 [SMILES: CC(C)(C)OC(=O)NCCC(=O)O] | 3-((tert- butoxycarbonyl)amino) propanoic acid | Commercially available (CAS 3303-84-2) |
| Biotin-NHS ester | 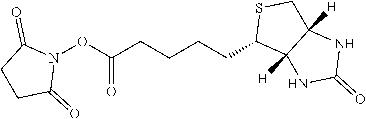 [SMILES: O=C1N[C@H]2[C@H](CS[C@H]2CCCCC(=O)ON2C(=O)CCC2=O)N1] | 2,5-dioxopyrrolidin-1- yl 5-((3aS,4S,6aR)-2- oxohexahydro-1H- thieno[3,4-d]imidazol- 4-yl)pentanoate | Commercially available (CAS 35013-72-0) |

### Example II. Analysis of Peptide Affinity and Potency Against HER2

### 11 Biological Data

### 11.1 Surface Plasmon Resonance (SPR) for Assessment of Peptide Affinity: Human & Mouse HER2

Peptide affinities (K_{D}) were determined by SPR using a Biacore™ 8K device (Cytiva) towards the proteins expressed in mammalian cells produced internally or purchased from external vendors: in house biotinylated His, Avi tag, human HER2 (23-652); biotinylated His, Avi tag, human HER2 (1-652, PDMU080) provided by PeptiDream Japan; in house biotinylated His tag, Avi tag, mouse HER2 (23-653). Each protein was immobilized either onto a SA sensorchip (Cytiva, BR-1005-31) or CM5 sensorchip (Cytiva, BR-1005-30) to a density of 800-2000 RU. The running buffer HBS-EP+ pH 7.6 (20× from Teknova CAT.No: H8022) was containing 10 mM HEPES pH 7.6, 150 mM NaCl, 3 mM EDTA, 0.05% Tween20 and 2% DMSO. Experiments were carried out at 25° C. using a flow rate of 30 uL/min. Compounds were tested in single cycle kinetic mode at 8 different concentrations. Curve fitting was performed using the Biacore™ 8K evaluation software. The sensorgrams were fitted by applying a 1:1 binding model to calculate kinetic rate constants and equilibrium dissociation constants (K_{D}).

When peptides reached the pM range of affinity combined with slow dissociation rates, the Fc capture method was used alternatively to increase the throughput for human HER2 SPR. For the assay setup the same conditions (flow rate, running buffer, peptide dilution) as described above were used. In a first step the Anti Fc IgG Ab (from Human Antibody Capture Kit, Ref. 29234600, Cytiva) was diluted into acetate pH 5.0 at 25 ug/mL and immobilized at a flow rate of 10 uL/min over 360 s to reach a density around 6000 RU. The human HER2 construct His, Fc tag, human HER2 (23-652, 1129-ER) purchased from R&D System was diluted at 33.3 ug/mL into 10 mM HEPES pH 7.6, 150 mM NaCl, 3 mM EDTA, 0.05% Tween20 and 2% DMSO and used with a contact time up to 300 s at a flow rate of 10 uL/min to reach a density of 200-500 RU. The peptides were tested with an association time of 280 s followed by a dissociation time of 3000 s at a flow rate of 30 uL/min. The regeneration was done using 3 M magnesium chloride regeneration solution as obtained with a contact time of 30 s at a flow rate of 10 uL/min. Curve fitting was performed using the Biacore™ 8K evaluation software. The sensorgrams were fitted by applying a 1:1 binding model to calculate kinetic rate constants and equilibrium dissociation constants (K_{D}).

### 11.2 Time Resolved-Fluorescence Energy Transfer (TR-FRET) for Assessment of Peptide Potency: Human & Mouse HER2

Peptide binding potencies (IC₅₀) were determined by TR-FRET competition assay towards human HER2 (23-652) and mouse HER2 (23-653) using a Cy5 labeled peptide as a probe. Both proteins containing a His-, Avi-tag and biotin in Nterminus, were expressed in mammalian cells and produced in house. The Cy5-peptide probe with similar motif as the tested peptides showed an affinity of 1-2 nM towards human and mouse HER2 (determined by fluorescence polarization) and was produced internally. Standard assay conditions consist of 20 uL total volume in white 384-well plates (Greiner), in 10 mM HEPES buffer containing 150 mM NaCl, 3 mM EDTA, 0.05% Tween20 and 1% final DMSO. Tested peptides at 14 different concentrations were added to 0.1 nM human or mouse HER2, 1 nM europium labeled streptavidin (Perkin Elmer) and 0.5 nM Cy5 labeled peptide probe. Samples were then incubated at 22-24° C. for 24 h before reading (excitation wavelength at 340 nm; emission wavelengths at 620 and 665 nm). The ratiometric FRET assay readout is calculated from the ratio of the raw data of the two distinct fluorescence signals measured in time resolved mode (fluorescence 665 nm/fluorescence 620 nm). IC₅₀ values were calculated with the standard Novartis in-house assay data analysis software (Helios software application, Novartis Institutes for BioMedical Research, unpublished) using the methods described in Comput. Methods. Programs. Biomed. 2006, 82, 31-37 (regression algorithms for nonlinear dose-response curve fitting).

TABLE 11.1
Summary of biological activities, in vitro,
as tested for compounds of the disclosure

| | huHER2-SPR- | moHER2- | huHER2 TR- | moHER2 TR- |
|---|---|---|---|---|
| | ONC | SPR-ONC | FRET | FRET |
| Example | K_{D} | K_{D} | IC₅₀ | IC₅₀ |
| No. | (nM) | (nM) | (nM) | (nM) |
| A1 | 97 | 98 | 111 | 224 |
| A2 | 0.4 | 0.2 | 0.3 | 0.3 |
| A3 | 1.3 | 2.4 | 1.4 | 3.2 |
| A4 | 8.9 | 12 | 14 | 29 |
| A5 | 0.5 | 0.6 | 0.4 | 0.8 |
| A6 | 2.4 | | 1.3 | 2.9 |
| A7 | 18 | 23 | 18 | 40 |
| A8 | 1.0 | | 1.2 | 2.3 |
| A9 | 117 | 243 | 75 | 172 |
| A10 | 482 | 520 | 536 | 684 |
| A11 | 445 | 786 | 966 | >1000 |
| A12 | 1344 | 864 | >1000 | >1000 |
| A13 | 40 | 44 | 46 | 107 |
| A14 | 1.3 | 1.2 | 1.7 | 3.7 |
| A15 | 24 | 24 | 27 | 55 |
| A16 | >6000 | >6000 | >1000 | >1000 |
| A17 | >6000 | >6000 | >1000 | >1000 |
| A18 | >6000 | >6000 | >1000 | >1000 |
| A19 | no binding* | no binding* | >1000 | >1000 |
| A20 | 1.8 | 2.5 | 2.2 | 4.7 |
| A21 | >6000 | >6000* | >1000 | >1000 |
| A22 | 9.7 | | 5.7 | 9.9 |
| A23 | 4.5 | 2.7 | 3 | 5.5 |
| A24 | 2810 | 5084 | >1000 | >1000 |
| A25 | 96 | 93 | 82 | 162 |
| A26 | 11 | 8.4 | 14 | 13 |
| A27 | 248 | 210 | 391 | 627 |
| A28 | 309 | 421 | 223 | 413 |
| A29 | 30 | 27 | 25 | 53 |
| A30 | 31 | 30 | 51 | 67 |
| A31 | 485 | 755 | 844 | >1000 |
| A32 | 1.4 | 2.2 | 1.2 | 2.6 |
| A33 | 15 | 23 | 14 | 32 |
| A34 | 68 | 64 | 158 (118)** | 296 (175)** |
| A35 | 37 | 66 | 53 | 122 |
| A36 | 38 | 34 | 52 | 102 |
| A37 | 452 | 213 | 810 | >1000 |
| A38 | 46 | 67 | 93 | 206 |
| A39 | 148 | 162 | 129 | 436 |
| A40 | <0.1 | <0.1 | <0.1 | 0.2 |
| A43 | 0.2 | | <0.1 | 0.2 |
| A45 | >6000 | >6000 | >900 | >1000 |
| A48 | | | 8298 | >10000 |
| A50 | 117 | 122 | 150 | 430 |
| A51 | 7.3 | 10 | 4.5 | 15 |
| A52 | 997 | 570 | 427 (567)** | >1000 |
| A53 | 132 | 154 | 163 | 466 |
| A54 | 1711 | 5305 | >1000 | >1000 |
| A55 | 178 | 148 | 97 | 287 |
| A56 | 443 | 413 | 229 | 755 |
| A57 | 2004 | 1462 | >1000 | >1000 |
| A74 | 0.1 | 0.1 | 0.1 | 0.5 (0.3)** |
| B1 | <0.1 | <0.1 | <0.1 | <0.1 |
| B2 | <0.1 | 0.1 | <0.1 | 0.2 |
| B3 | >6000 | >6000 | >1000 | >1000 |
| B4 | >6000 | >6000 | >1000 | >1000 |
| B5 | <0.1 | 0.1 | <0.1 | 0.2 |
| B6 | 0.5 | 1.2 | 0.3 | 1.1 |
| B7 | 0.4 | 0.8 | 0.4 | 1.3 |
| B8 | <0.1 | <0.1 | 0.1 | 0.3 |
| B9 | 5109 | >6000 | >1000 | >1000 |
| B10 | <0.1 | | <0.1 | <0.1 |
| B11 | <0.1 | | <0.1 | 0.2 |
| B12 | <0.1 | | <0.1 | <0.1 |
| B13 | <0.1 | <0.1 | <0.1 | <0.1 |
| C1 | <0.1 | <0.1 | <0.1 | 0.2 |
| C2 | <0.1 | 0.2 | <0.1 | 0.2 |
| C3 | >6000 | >6000 | >9500 | >10000 |
| C4 | <0.1 | | 0.1 | 0.4 |
| C5 | 0.7 | | 0.5 | 0.7 |
| C6 | 0.3 | | 0.5 (0.3)** | 1.5 (1)** |
| C7 | 1154 (794)** | 799 | 236 (164)** | 422 (434)** |
| C8 | 2.6 | | 2.1 | 3.3 |
| C9 | <0.1 | | <0.1 | 0.2 |
| C10 | <0.1 | | <0.1 | <0.1 |
| C11 | <0.1 | <0.1 | <0.1 | 0.1 |
| C12 | 0.4 | 1.4 | 0.3 | 1 |
| C13 | <0.1 | | <0.1 | 0.1 |
| C14 | <0.1 | 0.2 | <0.1 | 0.3 |
| C15 | <0.1 | <0.1 | <0.1 | 0.2 |
| C16 | <0.1 | | <0.1 | 0.1 |
| C17 | <0.1 | | <0.1 | 0.1 |
| C18 | 0.1 | | 0.1 | 0.6 |
| C19 | 5.5 | 10 | 2.9 | 7.6 |
| C20 | <0.1 | | 0.1 | 0.3 |
| C21 | 0.3 | 0.7 | 0.3 | 1.2 |
| C22 | 0.5 | | 0.4 | 1.8 |
| C23 | 47 | 39 | 62 (51)** | 76 (49)** |
| C24 | 12 | 9.3 | 13 (11)** | 13 |
| C25 | 0.6 | 2 | 1.5 (0.9)** | 6.1 (3.7)** |
| C26 | 5.1 | 11 | 4.2 | 17 |
| C27 | 32 | 54 | 23 | 57 |
| C28 | 0.6 | 3.1 | 1.8 (1.3)** | 4.4 |
| C29 | <0.1 | <0.1 | <0.1 | 0.2 |
| C30 | <0.1 | <0.1 | <0.1 | 0.2 |
| C31 | >6000 | >6000 | 4843 | >10000 |
| C32 | >6000 | >6000 | 9815 | >10000 |
| C33 | <0.1 | <0.1 | <0.1 | <0.1 |
| C34 | <0.1 | | <0.1 | <0.1 |
| C35 | <0.1 | <0.1 | <0.1 | <0.1 |
| C36 | 107 | 472 | 116 | 396 |
| C37 | <0.1 | 0.3 | 0.1 | 0.4 |
| D1 | <0.1 | | | |
| D2 | >6000 | | | |
| D3 | 26 | | | |
| D4 | 0.4 | | | |
| D5 | 1.5 | | | |
| D6 | 0.1 | | | |
| D7 | <0.1 | | | |
| F1 | <0.1 | 0.3 | <0.1 | 0.2 |
| F2 | <0.1 | <0.1 | <0.1 | 0.2 |
| F3 | <0.1 | | <0.1 | 0.3 |
| F4 | <0.1 | 0.1 | <0.1 | 0.3 |
| G1 | 0.7 | 1.5 | 1 | 3.8 |

*No binding was observed at concentrations up to 200 nM, which was the highest concentration tested due to limited availability.
**Recalculated value achieved after further repetition.

Affinities (SPR K_{D}) and potencies (TR-FRET IC₅₀) were performed on the extracellular domain of HER2, demonstrating that the tested HER2 peptides exhibit varying degrees of binding strength to this extracellular region of the protein. Notably, several of these peptides achieved remarkably high affinity, reaching into the picomolar range.

Several compounds (e.g., B1, B2, B4, C1, C2, and C29) were tested for selectivity over close protein homologs using a SPR method such as that described above for HER2, except that the immobilization of the protein homologs was done on a CM5 chip. The compounds showed selectivity for human HER2, as they did not show significant binding to human Herstatin (a soluble truncated HER2 protein) or other members of the ErbB family, including human EGFR, HER3, and HER4 proteins (all K_{D}>6000 nM).

### In Vivo Biological Data

### 11.3 In Vivo Biodistribution Experiments

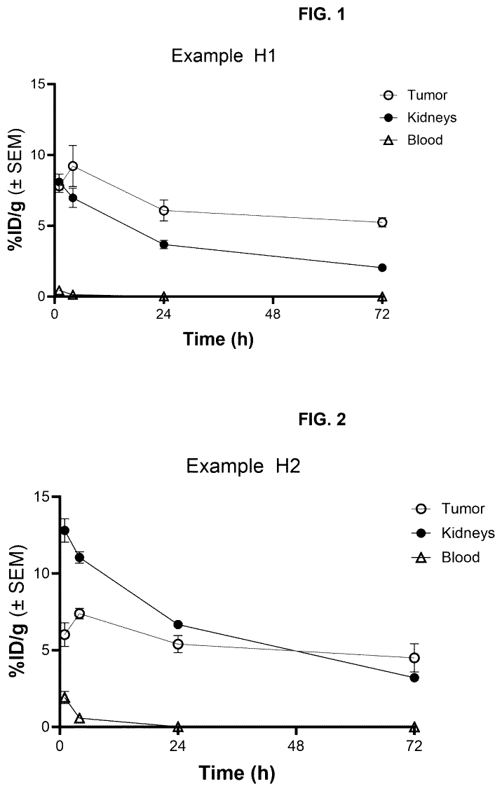

Biodistribution studies were performed at Minerva Imaging. All animal experimentation was carried out under a license approved by the National Animal Experiments Inspectorate under the Ministry of Environment and Food of Denmark, and further approved by the Novartis Animal Welfare Office. Biodistribution studies were performed in Nu (NCr)-Foxnlnu-homozygous mice bearing subcutaneous JimT1 tumors. Mice were subcutaneously implanted with 2×10⁶ JimT1 cells into right flank. The biodistribution studies were typically performed when tumors were approximately 200 mm³ in volume. Compounds according to Examples H1 and H2 were prepared as described in the radiochemistry part (Section 9). Animals were single dose administered with an activity concentration of 4 MBq/nmol per animal. The biodistribution of the compounds was assessed using conventional ex vivo biodistribution at different time points (1, 4, 24 and 72 hrs) after intravenous injection of Lu-177 labelled compounds. Mice were euthanized and organs were collected, weighed, and placed inside counting vials. Organ uptake was assessed by gamma counting using an automated gamma counter for 60 seconds immediately after tissue collection. Tissue counts and injected doses for individual animals were decay-corrected to the time of injection. Four animals were used per time point. Results expressed as a percentage of injected dose per gram of the tissue (% ID/g). The results of the biodistribution studies (tumor, kidney and blood uptake) are presented in FIG. 1 and FIG. 2. The tested compounds demonstrate high tumor uptake and retention and favorable tumor/normal tissue ratios.

### 11.4 In Vivo Efficacy Experiments

For all efficacy studies performed at Minerva Imaging: animal experimentation was carried out under a license approved by the National Animal Experiments Inspectorate under the Ministry of Environment and Food of Denmark, and further approved by the Novartis Animal Welfare Office. ¬¬ All efficacy studies performed at Novartis were conducted in accordance with Cantonal Veterinary Office of Basel-City and strictly adhered to the Federal Animal Protection Act and the Federal Animal Welfare Ordinance.

### HCC1187 Breast Cancer CDX Tumor Model:

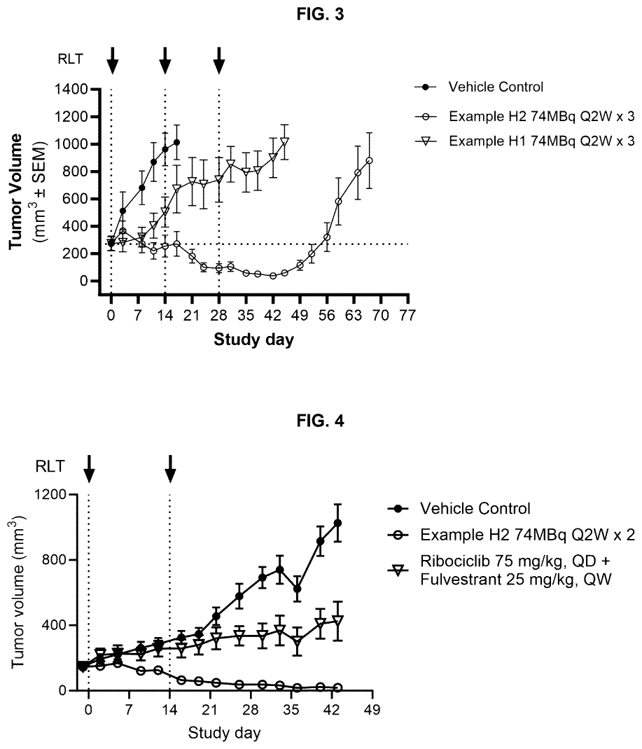

Antitumor efficacy studies were performed in Nu (NCr)-Foxnlnu-homozygous mice bearing subcutaneous HCC1187 breast CDX tumors. Mice were subcutaneously implanted with 5×10⁶ HCC1187 cells into the right flank. The studies were typically performed when tumors were approximately 200-300 mm³ in volume. Compounds according to Examples H1 and H2 were prepared as described in the radiochemistry part (Section 9). Animals were administered with an activity concentration of 74 MBq/nmol per animal every two weeks, 3 times, as indicated by the dotted lines. The efficacy was evaluated by tumor growth monitoring, mean and SEM shown in FIG. 3 (7 animals were used per group).

### ST313 Breast Cancer PDX Tumor Model:

Antitumor efficacy studies were performed in NMRI mice bearing the subcutaneous ST313 ER⁺ Breast Cancer PDX model. Mice were subcutaneously implanted with a PDX fragment into the right flank. The studies were typically performed when tumors were approximately 150 mm³ in volume. Example H2 was prepared as described in the radiochemistry part (Section 9). Animals were administered with an activity concentration of 74 MBq/nmol per animal every second week, two times, as indicated by the dotted lines. Another group was also administered 75 mg/kg Ribociclib daily together with 25 mg/kg Fulvestrant weekly. The efficacy was evaluated by tumor growth monitoring, mean and SEM shown in FIG. 4 (8 animals were used per group).

### ST1243 NSCLC PDX Tumor Model:

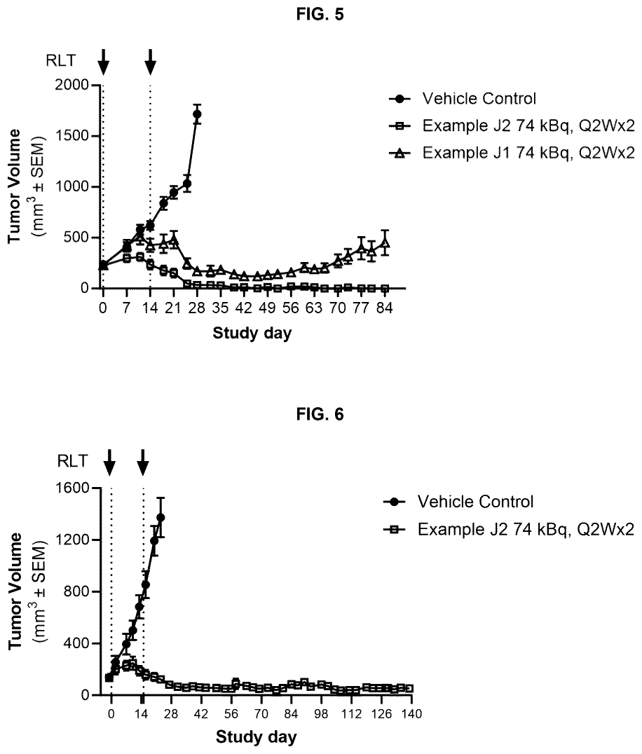

Antitumor efficacy studies were performed in NMRI mice bearing the subcutaneous ST1243 NSCLC PDX model. Mice were subcutaneously implanted with a PDX fragment into the right flank. The studies were typically performed when tumors were approximately 150-250 mm³ in volume. Examples J1 and J2 were radiolabeled as described in the radiochemistry part (Section 9). Animals were administered with an activity concentration of 74 MBq/nmol per animal every second week for a total of two weeks as indicated by the dotted lines. The efficacy was evaluated by tumor growth monitoring, mean and SEM shown in FIG. 5. (8 animals were used per group).

### ST3932 Breast Cancer PDX Tumor Model:

Antitumor efficacy studies were performed in NMRI mice bearing the subcutaneous ST3932 ER Breast Cancer PDX model. Mice were subcutaneously implanted with a PDX fragment into the right flank. The studies were typically performed when tumors were approximately 150-250 mm³ in volume. Example J2 was radiolabeled as described in the radiochemistry part (Section 9). Animals were administered with an activity concentration of 74 MBq/nmol per animal every second week for a total of two weeks as indicated by the dotted lines. The efficacy was evaluated by tumor growth monitoring, mean and SEM shown in FIG. 6. (8 animals were used per group).

These studies show the compounds of the instant disclosure are efficacious against several tumor models.

### Example III. Table of Compounds

| Ex. No. | A¹ | A² | A³ | A⁴ | A⁵ |
|---|---|---|---|---|---|
| A1 | NAc-E | 3PyA(6OH) | S | NMehF | NMeS |
| A2⁵ | NAc-hE | Y | S | NMehF | MeKAc |
| A3 | NAc-hE | Y | S | NMehF | MeKAc |
| A4 | NAc-hE | Y | S | NMehF | NMe-K(TFA) |
| A5 | NAc-hE | Y | S | NMehF | MeKAc |
| A6 | NAc-hE | Y | S | NMehF | MeKAc |
| A7 | H-GlutarA | Y | S | NMehF | MeKAc |
| A8 | NAc-hE | Y | S | NMehF (3Cl) | MeKAc |
| A9 | NAc-Asp | Y | S | NMehF | NMeS |
| A10 | NAc-E | Y | S | NMehF | NMeS |
| A11 | NAc-hE | Y | S | NMehF | MeKAc |
| A12 | NAc-E | Y | S | NMehF | NMeS |
| A13 | NAc-hE | Y | S | NMehF | MeKAc |
| A14 | NAc-hE | Y | S | NMeAhp | MeKAc |
| A15 | NAc-hE | Y | S | NMehF | MeKAc |
| A16 | NAc-E | Y | S | NMehF | NMeS |
| A17 | NAc-hE | Y | S | NMehF | MeKAc |
| A18 | NAc-hE | Y | S | NMehF | MeKAc |
| A19 | NAc-Dap | Y | S | NMehF | NMeS |
| A20 | NAc-hE | Y | S | NMeAoc | MeKAc |
| A21 | NAc-E | Y | S | NMehF | NMeS |
| A22 | H-bA(2S-Me) | Y | S | NMehF | MeKAc |
| A23 | H-bA(2S-OH) | Y | S | NMehF | MeKAc |
| A24 | 3-(CH2NH2)Bz | Y | S | NMehF | NMeS |
| A25 | NAc-E | Y | S | NMehF | NMeS |
| A26⁵ | NAc-hE | Y | S | NMehF | NMeS |
| A27 | NAc-E | Y | hE | NMehF | NMeS |
| A28 | NAc-Asp | Y | S | NMehF | NMeS |
| A29 | NAc-hE | Y | S | NMehF | NMeS |
| A30⁵ | NAc-E | Y | S | NMehF | NMeS |
| A31 | H-F(3aa) | Y | S | NMehF | NMeS |
| A32 | H-E | Y | S | NMehF | MeKAc |
| A33 | H-E | Y | S | NMehF | NMe-K(TFA) |
| A34 | H-Dap | Y | S | NMehF | NMeS |
| A35 | H-E | Y | S | NMehF | NMeS |
| A36 | H-hE | Y | S | NMehF | NMeS |
| A37 | H-Asp | Y | S | NMehF | NMeS |
| A38 | H-E | Y | NMeS | NMehF | NMeS |
| A39 | beta2E | Y | S | NMehF | NMeS |
| A40⁵ | NAc-hE | Y | S | NMehF | MeKAc |
| A41⁵ | NAc-hE | Y | S | NMehF | MeKAc |
| A42⁵ | NAc-hE | Y | KCOpipzaa | NMehF | MeKAc |
| A43⁵ | mPEG12-hE | Y | S | NMehF | MeKAc |
| A44⁵ | mPEG12-hE | Y | S | NMehF | MeKAc |
| A45⁵ | NAc-hE | Y | S | NMehF | MeKAc |
| A46⁵ | HOAc-hE | Y | S | NMehF | MeKAc |
| A47⁵ | NAc-hE | Y | S | NMehF | MeKAc |
| A48⁵ | NAc-hE | dY | dS | NMehF | MeKAc |
| A49⁵ | NAc-S(aa) | Y | S | NMehF | MeKAc |
| A50 | NAc-Dap(ClAc) | Y | S | NMehF | NMeS |
| A51 | NAc-Dap(ClAc) | Y | S | NMehF | MeKAc |
| A52 | H-Alk1 | Y | S | NMehF | NMeS |
| A53 | H-Dap(ClAc) | Y | S | NMehF | NMeS |
| A54 | H-Cys | Y | S | NMehF | NMeS |
| A55 | H-hCys | Y | S | NMehF | MeKAc |
| A56 | H-Cys | Y | S | NMehF | MeKAc |
| A57 | H-hCys | Y | S | NMehF | NMeS |
| A58 | H-hE | Y | S | NMehF | MeKAc |
| A59 | H-PEG10-hE | Y | S | NMehF | MeKAc |
| A60⁵ | Malonyl-hE | Y | S | NMehF | MeKAc |
| A61⁶ | NAc-hE | Y | S | NMehF | MeKAc |
| A62⁵ | AdipicA | Y | S | NMehF | MeKAc |
| A63⁵ | Succinyl-hE | Y | S | NMehF | MeKAc |
| A64⁵ | COCH2Tet(1H)-hE | Y | S | NMehF | MeKAc |
| A65⁵ | NAc-hE | Y | S | NMehF | MeK(COCH2OH) |
| A66⁵ | NAc-hE | Y | S | NMehF | MeK(COCH2OH) |
| A67⁵ | NAc-hE | Y | S | NMehF | MeK(COEtOH) |
| A68², 11 | NAc-E | Y | S | NMehF | MeKAc |
| A69², 10 | NAc-E | Y | S | NMehF | MeKAc |
| A70⁵ | NAc-E | Y | S | NMehF | MeKAc |
| A71⁵ | NAc-E | Y | Dap | NMehF | MeKAc |
| A72⁵ | H-E | Y | S | NMehF | MeKAc |
| A73⁵ | bA-E | Y | S | NMehF | MeKAc |
| A74⁵ | NAc-hE | Y | S | NMehF | MeKAc |
| A75⁵ | NAc-hE | Y | S | NMehF | MeKAc |
| A76⁵ | NAc-hE | Y | S | NMehF | MeKAc |
| A77⁵ | NAc-hE | Y | S | NMehF | MeKAc |
| B1⁵ | NAc-hE | Y | S | NMehF | MeKAc |
| B2⁵ | mPEG12-hE | Y | S | NMehF | MeKAc |
| B3⁵ | NAc-hE | Y | S | NMehF | MeKAc |
| B4⁵ | NAc-hE | dY | dS | NMehF | MeKAc |
| B5⁵ | NAc-hE | Y | S | NMehF | MeK(COCH2OH) |
| B6⁵ | NAc-hE | Y | S | NMehF | MeK(COCH2OH) |
| B7⁵ | NAc-hE | Y | S | NMehF | MeK(COEtOH) |
| B9⁵ | NAc-hE | Y | S | NMehF | MeKAc |
| B10⁵ | NAc-hE | Y | S | NMehF | MeKAc |
| B11⁵ | mPEG12-hE | Y | S | NMehF | MeKAc |
| B12⁵ | NAc-hE | Y | S | NMehF | MeKAc |
| B13⁵ | NAc-hE | Y | S | NMehF | MeKAc |
| C1⁵ | NAc-hE | Y | S | NMehF | MeKAc |
| C2⁵ | mPEG12-hE | Y | S | NMehF | MeKAc |
| C5 | PEG10(DOTA-Lu)-hE | Y | S | NMehF | MeKAc |
| C6⁵ | NAc-hE | Y | S | NMehF | MeKAc |
| C7⁵ | NAc-hE | Y | KCOpipzaa | NMehF | MeKAc |
| C8 | DOTA-Lu-hE | Y | S | NMehF | MeKAc |
| C9⁵ | NAc-hE | Y | S | NMehF | MeKAc |
| C10⁵ | Malonyl-hE | Y | S | NMehF | MeKAc |
| C11⁵ | Succinyl-hE | Y | S | NMehF | MeKAc |
| C12⁶ | NAc-hE | Y | S | NMehF | MeKAc |
| C13⁵ | COCH2Tet(1H)-hE | Y | S | NMehF | MeKAc |
| C14⁵ | AdipicA | Y | S | NMehF | MeKAc |
| C15⁵ | HOAc-hE | Y | S | NMehF | MeKAc |
| C16⁵ | NAc-S(aa) | Y | S | NMehF | MeKAc |
| C17⁵ | NAc-hE | Y | S | NMehF | MeKAc |
| C18⁵ | H-hE | Y | S | NMehF | MeKAc |
| C19⁵ | NAc-hE | Y | S | NMehF | MeK(Malonyl) |
| C20⁵ | NAc-hE | Y | S | NMehF | MeK(COCH2OH) |
| C21⁵ | NAc-hE | Y | S | NMehF | MeK(COCH2OH) |
| C22⁵ | NAc-hE | Y | S | NMehF | MeK(COEtOH) |
| C23², ⁷ | NAc-E | Y | S | NMehF | MeKAc |
| C24², ⁸ | NAc-E | Y | S | NMehF | MeKAc |
| C25⁵ | NAc-E | Y | S | NMehF | MeKAc |
| C26⁵ | NAc-E | Y | Dap(DOTA-Lu) | NMehF | MeKAc |
| C27⁵ | DOTA-Lu-E | Y | S | NMehF | MeKAc |
| C28⁵ | b-A(DOTA-Lu)E | Y | S | NMehF | MeKAc |
| C29⁵ | NAc-hE | Y | S | NMehF | MeKAc |
| C30⁵ | NAc-hE | Y | S | NMehF | MeKAc |
| C31⁵ | NAc-hE | Y | S | NMehF | MeKAc |
| C32⁵ | NAc-hE | Y | S | NMehF | MeKAc |
| C33⁵ | NAc-hE | Y | S | NMehF | MeKAc |
| C34⁵ | NAc-hE | Y | S | NMehF | MeKAc |
| C35⁵ | NAc-hE | Y | S | NMehF | MeKAc |
| C36⁵ | NAc-hE | dY | dS | NMehF | MeKAc |
| C37⁵ | mPEG12-hE | Y | S | NMehF | MeKAc |
| D1⁵ | NAc-hE | Y | S | NMehF | MeKAc |
| D2⁵ | NAc-hE | Y | S | NMehF | MeKAc |
| D3¹, ³, ⁹ | NAc-E | Y | S | NMehF | MeKAc |
| D4⁵ | NAc-E | Y | S | NMehF | MeKAc |
| D5⁵ | b-Ala(Cy5)-E | Y | S | NMehF | MeKAc |
| D6⁵ | PEG10(Cy5)-hE | Y | S | NMehF | MeKAc |
| D7⁵ | NAc-hE | Y | S | NMehF | MeKAc |
| E1⁵ | NAc-E | Y | S | NMehF | MeKAc |
| E2¹, ⁴, ⁹ | NAc-E | Y | S | NMehF | MeKAc |
| E3⁵ | b-A(PEG10Biotin)-E | Y | S | NMehF | MeKAc |
| E4⁵ | NAc-hE | Y | S | NMehF | MeKAc |
| E5⁵ | NAc-hE | Y | S | NMehF | MeKAc |
| F1⁵ | NAc-hE | Y | S | NMehF | MeKAc |
| F2⁵ | NAc-hE | Y | S | NMehF | MeKAc |
| F3⁵ | mPEG12-hE | Y | S | NMehF | MeKAc |
| F4⁵ | mPEG12-hE | Y | S | NMehF | MeKAc |
| G1⁵ | BB7-hE | Y | S | NMehF | MeKAc |
| H1⁵ | mPEG12-hE | Y | S | NMehF | MeKAc |
| H2⁵ | NAc-hE | Y | S | NMehF | MeKAc |
| H3 | NAc-hE | Y | S | NMehF | MeKAc |
| H4 | NAc-hE | dY | dS | NMehF | MeKAc |
| I1⁵ | mPEG12-hE | Y | S | NMehF | MeKAc |
| I2⁵ | NAc-hE | Y | S | NMehF | MeKAc |
| J1⁵ | mPEG12-hE | Y | S | NMehF | MeKAc |
| J2⁵ | NAc-hE | Y | S | NMehF | MeKAc |

| Ex. No. | A⁶ | A⁷ | A⁸ | A⁹ | A¹⁰ | A¹⁰* | SEQ ID NO |
|---|---|---|---|---|---|---|---|
| A1 | W(5OH) | W | CproG | NMeDap | NMeF(4F) | NH2 | |
| A2⁵ | W(5OH) | W | CproG | NMeDap | NMeF(4F) | Pro | 1 |
| A3 | W(5OAc) | W | CproG | NMeDap | NMeF(4F) | NH2 | |
| A4 | W(5OH) | W | CproG | NMeDap | NMeF(4F) | NH2 | |
| A5 | W(5OH) | W | CproG | NMeDap | NMeF(4F) | NH2 | |
| A6 | W(5OH) | W | AlloT | NMeDap | NMeF(4F) | NH2 | |
| A7 | W(5OH) | W | CproG | NMeDap | NMeF(4F) | NH2 | |
| A8 | W(5OH) | W | CproG | NMeDap | NMeF(4F) | NH2 | |
| A9 | W(5OH) | W | CproG | NMeDab | NMeF(4F) | NH2 | 2 |
| A10 | W(5OH) | W | CproG | NMeDab | NMeF(4F) | NH2 | 3 |
| A11 | Ahp | W | cProGG | NMeDap | NMeF(4F) | NH2 | |
| A12 | W(5OH) | W | cProGG | Dap | NMeF(4F) | NH2 | 4 |
| A13 | W(5OH) | W | cProGG | NMeDap | NMeAoc | NH2 | |
| A14 | W(5OH) | W | cProGG | NMeDap | NMeF(4F) | NH2 | |
| A15 | W(5OH) | W | cProGG | NMeDap | NMeAhp | NH2 | |
| A16 | W8N | W | cProGG | NMeDap | NMeF(4F) | NH2 | 5 |
| A17 | W(5OH) | Ahp | cProGG | NMeDap | NMeF(4F) | NH2 | |
| A18 | W(5OH) | Aoc | cProGG | NMeDap | NMeF(4F) | NH2 | |
| A19 | W(5OH) | W | cProGG | NMeE | NMeF(4F) | NH2 | |
| A20 | W(5OH) | W | cProGG | NMeDap | NMeF(4F) | NH2 | |
| A21 | W(5OH) | W8N | cProGG | NMeDap | NMeF(4F) | NH2 | |
| A22 | W(5OH) | W | CproG | NMeE | NMeF(4F) | NH2 | |
| A23 | W(5OH) | W | CproG | NMeE | NMeF(4F) | NH2 | |
| A24 | W(5OH) | W | cProGG | NMeD | NMeF(4F) | NH2 | |
| A25 | W(5OH) | W | CproG | NMeDap | NMeF(4F) | NH2 | 6 |
| A26⁵ | W(5OH) | W | CproG | NMeDap | NMeF(4F) | Pro | 7 |
| A27 | W(5OH) | W | CproG | NMeDap | NMeF(4F) | NH2 | |
| A28 | W(5OH) | W | CproG | NMeDap | NMeF(4F) | NH2 | 8 |
| A29 | W(5OH) | W | CproG | NMeDap | NMeF(4F) | NH2 | |
| A30⁵ | W(5OH) | W | CproG | NMeDap | NMeF(4F) | Pro | 9 |
| A31 | W(5OH) | W | cProG | NMeDap | NMeF(4F) | NH2 | |
| A32 | W(5OH) | W | CproG | NMeDap | NMeF(4F) | NH2 | 10 |
| A33 | W(5OH) | W | CproG | NMeDap | NMeF(4F) | NH2 | 11 |
| A34 | W(5OH) | W | CproG | NMeE | NMeF(4F) | NH2 | |
| A35 | W(5OH) | W | CproG | NMeDap | NMeF(4F) | NH2 | 12 |
| A36 | W(5OH) | W | CproG | NMeDap | NMeF(4F) | NH2 | |
| A37 | W(5OH) | W | CproG | NMeDap | NMeF(4F) | NH2 | 13 |
| A38 | W(5OH) | W | CproG | NMeDap | NMeF(4F) | NH2 | |
| A39 | W(5OH) | W | CproG | NMeDap | NMeF(4F) | NH2 | |
| A40⁵ | W(5OH) | W | cProG | NMeDap | NMeF(4F) | Lys | 14 |
| A41⁵ | W(5OH) | W | AlloT | NMeDap | NMeF(4F) | Lys | 15 |
| A42⁵ | W(5OH) | W | CproG | NMeDap | NMeF(4F) | Lys | |
| A43⁵ | W(5OH) | W | cProG | NMeDap | NMeF(4F) | Lys | 16 |
| A44⁵ | W(5OH) | W | cProG | NMeDap | NMeF(4F) | K(N3) | |
| A45⁵ | W(5OH) | Ala | cProG | NMeDap | NMeF(4F) | Lys | 17 |
| A46⁵ | W(5OH) | W | CproG | NMeDap | NMeF(4F) | K(N3) | |
| A47⁵ | W(5OH) | W | CproG | NMeDap | NMeF(4F) | Orn | |
| A48⁵ | W(5OH) | W | CproG | NMeDap | NMeF(4F) | Lys | 18 |
| A49⁵ | W(5OH) | W | CproG | NMeDap | NMeF(4F) | K(N3) | |
| A50 | W(5OH) | W | CproG | NMeCys | NMeF(4F) | NH2 | |
| A51 | W(5OH) | W | CproG | NMeCys | NMeF(4F) | NH2 | |
| A52 | W(5OH) | W | cProG | NMeC | NMeF(4F) | NH2 | |
| A53 | W(5OH) | W | CproG | NMeCys | NMeF(4F) | NH2 | |
| A54 | W(5OH) | W | cProG | NMeC | NMeF(4F) | NH2 | 19 |
| A55 | W(5OH) | W | CproG | NMehCys | NMeF(4F) | NH2 | |
| A56 | W(5OH) | W | cProG | NMehC | NMeF(4F) | NH2 | 20 |
| A57 | W(5OH) | W | cProG | NMeC | NMeF(4F) | NH2 | |
| A58 | W(5OH) | W | CproG | NMeDap | NMeF(4F) | NH2 | |
| A59 | W(5OH) | W | CproG | NMeDap | NMeF(4F) | NH2 | |
| A60⁵ | W(5OH) | W | CproG | NMeDap | NMeF(4F) | Lys | 21 |
| A61⁶ | W(5OH) | W | CproG | NMeDap | NMeF(4F) | Lys | 22 |
| A62⁵ | W(5OH) | W | CproG | NMeDap | NMeF(4F) | Lys | 23 |
| A63⁵ | W(5OH) | W | CproG | NMeDap | NMeF(4F) | Lys | 24 |
| A64⁵ | W(5OH) | W | CproG | NMeDap | NMeF(4F) | Lys | 25 |
| A65⁵ | W(5OH) | W | CproG | NMeDap | NMeF(4F) | Lys | 26 |
| A66⁵ | W(5OH) | W | AlloT | NMeDap | NMeF(4F) | Lys | 27 |
| A67⁵ | W(5OH) | W | AlloT | NMeDap | NMeF(4F) | Lys | 28 |
| A68², ¹¹ | W(5OH) | W | AlloT | NMeDap | NMeF(4F) | KCOpipzaa | 29 |
| A69², ¹⁰ | W(5OH) | W | AlloT | NMeDap | NMeF(4F) | KCOpipzaa | 30 |
| A70⁵ | W(5OH) | W | AlloT | NMeDap | NMeF(4F) | Lys | 31 |
| A71⁵ | W(5OH) | W | AlloT | NMeDap | NMeF(4F) | KCOpipzaa | |
| A72⁵ | W(5OH) | W | AlloT | NMeDap | NMeF(4F) | KCOpipzaa | 32 |
| A73⁵ | W(5OH) | W | AlloT | NMeDap | NMeF(4F) | KCOpipzaa | 121 |
| A74⁵ | W(5OH) | W | cProG | NMeDap | NMeF(4F) | K(N3) | |
| A75⁵ | W(5OH) | dW | cProG | NMeDap | NMeF(4F) | Lys | 33 |
| A76⁵ | W(5OH) | W | cProG | NMeDap | NMeF(4F) | K(PEG10) | 34 |
| A77⁵ | W(5OH) | A | cProG | NMeDap | NMeF(4F) | K(PEG10) | 35 |
| B1⁵ | W(5OH) | W | CproG | NMeDap | NMeF(4F) | K(DOTA) | 36 |
| B2⁵ | W(5OH) | W | CproG | NMeDap | NMeF(4F) | K(DOTA) | 37 |
| B3⁵ | W(5OH) | Ala | cProG | NMeDap | NMeF(4F) | K(DOTA) | 38 |
| B4⁵ | W(5OH) | W | CproG | NMeDap | NMeF(4F) | K(DOTA) | 39 |
| B5⁵ | W(5OH) | W | CproG | NMeDap | NMeF(4F) | K(DOTA) | 40 |
| B6⁵ | W(5OH) | W | AlloT | NMeDap | NMeF(4F) | K(DOTA) | 41 |
| B7⁵ | W(5OH) | W | AlloT | NMeDap | NMeF(4F) | K(DOTA) | 42 |
| B9⁵ | W(5OH) | dW | CproG | NMeDap | NMeF(4F) | K(DOTA) | 43 |
| B10⁵ | W(5OH) | W | cProG | NMeDap | NMeF(4F) | K(NOTA) | 44 |
| B11⁵ | W(5OH) | W | CproG | NMeDap | NMeF(4F) | K(NOTA) | 45 |
| B12⁵ | W(5OH) | W | cProG | NMeDap | NMeF(4F) | K((R)-DOTAGA) | 46 |
| B13⁵ | W(5OH) | W | cProG | NMeDap | NMeF(4F) | K(NODAGA) | 47 |
| C1⁵ | W(5OH) | W | CproG | NMeDap | NMeF(4F) | K(DOTA-Lu) | 48 |
| C2⁵ | W(5OH) | W | CproG | NMeDap | NMeF(4F) | K(DOTA-Lu) | 49 |
| C5 | W(5OH) | W | CproG | NMeDap | NMeF(4F) | NH2 | |
| C6⁵ | W(5OH) | W | AlloT | NMeDap | NMeF(4F) | K(DOTA-Lu) | 50 |
| C7⁵ | W(5OH) | W | CproG | NMeDap | NMeF(4F) | K(DOTA-Lu) | |
| C8 | W(5OH) | W | CproG | NMeDap | NMeF(4F) | NH2 | |
| C9⁵ | W(5OH) | W | CproG | NMeDap | NMeF(4F) | Orn(DOTA-Lu) | |
| C10⁵ | W(5OH) | W | CproG | NMeDap | NMeF(4F) | K(DOTA-Lu) | 51 |
| C11⁵ | W(5OH) | W | CproG | NMeDap | NMeF(4F) | K(DOTA-Lu) | 52 |
| C12⁶ | W(5OH) | W | CproG | NMeDap | NMeF(4F) | K(DOTA-Lu) | 53 |
| C13⁵ | W(5OH) | W | CproG | NMeDap | NMeF(4F) | K(DOTA-Lu) | 54 |
| C14⁵ | W(5OH) | W | CproG | NMeDap | NMeF(4F) | K(DOTA-Lu) | 55 |
| C15⁵ | W(5OH) | W | CproG | NMeDap | NMeF(4F) | K(DOTA-Lu) | 56 |
| C16⁵ | W(5OH) | W | CproG | NMeDap | NMeF(4F) | K(DOTA-Lu) | 57 |
| C17⁵ | W(5OH) | W | CproG | NMeDap | NMeF(4F) | hK(DOTA-Lu) | |
| C18⁵ | W(5OH) | W | CproG | NMeDap | NMeF(4F) | K(DOTA-Lu) | 58 |
| C19⁵ | W(5OH) | W | CproG | NMeDap | NMeF(4F) | K(DOTA-Lu) | 59 |
| C20⁵ | W(5OH) | W | CproG | NMeDap | NMeF(4F) | K(DOTA-Lu) | 60 |
| C21⁵ | W(5OH) | W | AlloT | NMeDap | NMeF(4F) | K(DOTA-Lu) | 61 |
| C22⁵ | W(5OH) | W | AlloT | NMeDap | NMeF(4F) | K(DOTA-Lu) | 62 |
| C23², ⁷ | W(5OH) | W | AlloT | NMeDap | NMeF(4F) | KCOpipzaa | 63 |
| C24², ⁸ | W(5OH) | W | AlloT | NMeDap | NMeF(4F) | KCOpipzaa | 64 |
| C25⁵ | W(5OH) | W | AlloT | NMeDap | NMeF(4F) | K(DOTA-Lu) | 65 |
| C26⁵ | W(5OH) | W | AlloT | NMeDap | NMeF(4F) | KCOpipzaa | |
| C27⁵ | W(5OH) | W | AlloT | NMeDap | NMeF(4F) | KCOpipzaa | 66 |
| C28⁵ | W(5OH) | W | AlloT | NMeDap | NMeF(4F) | KCOpipzaa | 67 |
| C29⁵ | W(5OH) | W | CproG | NMeDap | NMeF(4F) | K(DOTA-Ga) | 68 |
| C30⁵ | W(5OH) | W | CproG | NMeDap | NMeF(4F) | K(DOTA-La) | 69 |
| C31⁵ | W(5OH) | dW | cProG | NMeDap | NMeF(4F) | K(DOTA-Lu) | 70 |
| C32⁵ | W(5OH) | A | CproG | NMeDap | NMeF(4F) | K(DOTA-Lu) | 71 |
| C33⁵ | W(5OH) | W | cProG | NMeDap | NMeF(4F) | K((R)-DOTAGA-Ga) | 72 |
| C34⁵ | W(5OH) | W | cProG | NMeDap | NMeF(4F) | K(NODAGA-Ga) | 73 |
| C35⁵ | W(5OH) | W | cProG | NMeDap | NMeF(4F) | K(NODAGA-Ga) | 74 |
| C36⁵ | W(5OH) | W | CproG | NMeDap | NMeF(4F) | K(DOTA-La) | 75 |
| C37⁵ | W(5OH) | W | CproG | NMeDap | NMeF(4F) | K(DOTA-La) | 76 |
| D1⁵ | W(5OH) | W | CproG | NMeDap | NMeF(4F) | K(Cy5) | 77 |
| D2⁵ | W(5OH) | A | CproG | NMeDap | NMeF(4F) | K(Cy5) | 78 |
| D3¹, ³, ⁹ | W(5OH) | W | AlloT | NMeDap | NMeF(4F) | KCOpipzaa | 79 |
| D4⁵ | W(5OH) | W | AlloT | NMeDap | NMeF(4F) | K(Cy5) | 80 |
| D5⁵ | W(5OH) | W | AlloT | NMeDap | NMeF(4F) | KCOpipzaa | 81 |
| D6⁵ | W(5OH) | W | CproG | NMeDap | NMeF(4F) | K(DOTA-Lu) | 82 |
| D7⁵ | W(5OH) | W | cProG | NMeDap | NMeF(4F) | K(PEG10sCy5) | 83 |
| E1⁵ | W(5OH) | W | AlloT | NMeDap | NMeF(4F) | K(PEG10biotin) | 84 |
| E2¹, ⁴, ⁹ | W(5OH) | W | AlloT | NMeDap | NMeF(4F) | KCOpipzaa | 85 |
| E3⁵ | W(5OH) | W | AlloT | NMeDap | NMeF(4F) | KCOpipzaa | 86 |
| E4⁵ | W(5OH) | W | cProG | NMeDap | NMeF(4F) | K(PEG10biotin) | 87 |
| E5⁵ | W(5OH) | A | cProG | NMeDap | NMeF(4F) | K(PEG10biotin) | 88 |
| F1⁵ | W(5OH) | W | CproG | NMeDap | NMeF(4F) | K(COPy3(4F)) | 89 |
| F2⁵ | W(5OH) | W | CproG | NMeDap | NMeF(4F) | K(CO3Py4(NME3)) | 90 |
| F3⁵ | W(5OH) | W | CproG | NMeDap | NMeF(4F) | K(COPy3(4F)) | 91 |
| F4⁵ | W(5OH) | W | CproG | NMeDap | NMeF(4F) | K(CO3Py4(NME3)) | 92 |
| G1⁵ | W(5OH) | W | CproG | NMeDap | NMeF(4F) | K(DOTA-Lu) | 93 |
| H1⁵ | W(5OH) | W | CproG | NMeDap | NMeF(4F) | K(DOTA-¹⁷⁷Lu) | 94 |
| H2⁵ | W(5OH) | W | CproG | NMeDap | NMeF(4F) | K(DOTA-¹⁷⁷Lu) | 95 |
| H3 | W(5OH) | Ala | cProG | NMeDap | NMeF(4F) | K(DOTA-¹⁷⁷Lu) | 96 |
| H4 | W(5OH) | W | CproG | NMeDap | NMeF(4F) | K(DOTA-¹⁷⁷Lu) | 97 |
| I1⁵ | W(5OH) | W | CproG | NMeDap | NMeF(4F) | K(DOTA-⁶⁸Ga) | 98 |
| I2⁵ | W(5OH) | W | CproG | NMeDap | NMeF(4F) | K(DOTA-⁶⁸Ga) | 99 |
| J1⁵ | W(5OH) | W | CproG | NMeDap | NMeF(4F) | K(DOTA-²²⁵Ac) | 100 |
| J2⁵ | W(5OH) | W | CProG | NMeDap | NMeF(4F) | K(DOTA-²²⁵Ac) | 101 |

¹A¹⁰**** is NH₂; ²A¹⁰*** is NH₂; ³A¹⁰*** is K(Cy5); ⁴A¹⁰*** is K(biotin); ⁵A¹⁰** is NH₂; ⁶A¹⁰** is OH; ⁷A¹⁰** is dK(DOTA-Lu); ⁸A¹⁰** is K(DOTA-Lu); ⁹A¹⁰** is PEG10; ¹⁰A¹⁰** is K; and ¹¹A¹⁰** is dK.

Those skilled in the art will recognize, or be able to ascertain, using no more than routine experimentation, numerous equivalents to the specific embodiments described specifically herein. Such equivalents are intended to be encompassed in the scope of the following claims.

1. A compound, or a pharmaceutically acceptable salt or solvate thereof, comprising
a) at least one cyclized peptide \textcircled{P}, wherein \textcircled{P} is 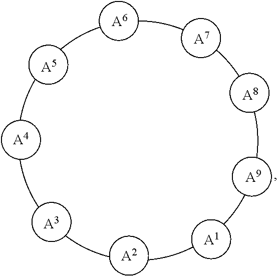
wherein:
A¹ is 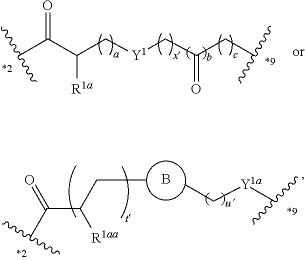
wherein:
R^{1a} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁-6-alkyl)₂, and —NH₂;
\textcircled{B} is selected from C₃₋₈-cycloalkyl, phenyl, and 5-6 membered heteroaryl, wherein the C₃₋₈-cycloalkyl, phenyl, and 5-6 membered heteroaryl of \textcircled{B} are optionally substituted with 1 or 2 substituents independently selected from halo, —OH, —NH₂, and C₁₋₆-alkyl;
R^{1aa} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁-6-alkyl)₂, and —NH₂;
Y¹ is selected from a bond, C≡C, NH, NC₁₋₆-alkyl, O, and S;
Y^{1a} is selected from C(O), NH, NC₁₋₆-alkyl, C(O)NH, C(O)NC₁₋₆-alkyl, NHC(O), N(C₁₋₆-alkyl) C(O), O, and S;
a is 1, 2, 3, or 4;
b, c, t′, and x′ are each independently 0 or 1;
u′ is 0, 1, 2, or 3; and
2 indicates the point of attachment to A² and *9 indicates the point of attachment to A⁹;
A² is 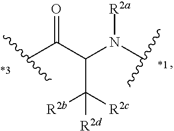
wherein:
R^{2a} is selected from H and C₁₋₆-alkyl;
R^{2b} is selected from H, C₁₋₆-alkyl, and halo;
R^{2c} is selected from halo, C₁₋₆-alkyl, C₃₋₈-cycloalkyl, phenyl, and 5-6 membered heteroaryl, wherein the C₁₋₆-alkyl, phenyl, and 5-6 membered heteroaryl of R^{2c} are optionally substituted with 1, 2, 3, or 4 substituents independently selected from halo, —C(O)NH₂, —C(O)OH, —C(O)C₁₋₆-alkyl, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —NHC₁₋₆-alkyl, —N(C₁₋₆-alkyl)₂, and —NH₂;
R^{2d} is selected from H, C₁₋₆-alkyl, and halo; and
3 indicates the point of attachment to A³ and *1 indicates the point of attachment to A¹;
A³ is 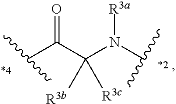
wherein:
R^{3a} is selected from H and C₁₋₆-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)—(5-6 membered heterocycloalkyl), and —C₁₋₆-alkyl-N(H)—C₁₋₆-alkyl-(5-6 membered heterocycloalkyl), wherein the C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)—(5-6 membered heterocycloalkyl), and —C₁₋₆-alkyl-N(H)—C₁₋₆-alkyl-(5-6 membered heterocycloalkyl) of R^{3c} are optionally substituted with 1, 2, 3, 4, 5, or 6 substituents independently selected from —CN, —C(O)OH, —C₁₋₆-alkyl-C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *4 indicates the point of attachment to A⁴ and *2 indicates the point of attachment to A²;
A⁴ is 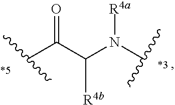
wherein:
R^{4a} is selected from H, C₁₋₆-alkyl, and —CH₂-phenyl, wherein the C₁₋₆-alkyl and —CH₂-phenyl of R^{4a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{4b} is selected from H, C₁₋₆-alkyl, —C₁₋₆-alkyl-phenyl, —C₁₋₆-alkyl-O-phenyl, —C₁₋₆-alkyl-(5-6 membered heteroaryl), —C₁₋₇-alkyl-C(O)OH, and —C₁₋₆-alkyl-NH—C(O)OH, wherein the C₁₋₆-alkyl, —C₁₋₆-alkyl-phenyl, —C₁₋₆-alkyl-O-phenyl, and —C₁₋₆-alkyl-(5-6 membered heteroaryl) of R^{4b} are optionally substituted with 1, 2, or 3 substituents independently selected from halo, —C(O)NH₂, —NH₂, —NHC₁₋₆-alkyl, —N(C₁₋₆-alkyl)₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —OC₁₋₆-alkyl, and C₁₋₆-alkyl; and
wherein *5 indicates the point of attachment to A⁵ and *3 indicates the point of attachment to A³;
A⁵ is 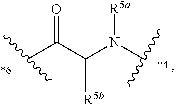
wherein:
R^{5a} is selected from H, C₁₋₆-alkyl, and —CH₂-phenyl, wherein the C₁₋₆-alkyl and —CH₂-phenyl of R^{5a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5b} is selected from H, C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c}, wherein the C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c} of R^{5b} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5c} is selected from H, C₁₋₆-alkyl, —NH₂, —NHC₁₋₆-alkyl, —N(C₁₋₆-alkyl)₂, —OH, —OC₁₋₆-alkyl, C₁-6-haloalkyl, C₁₋₆-alkyl-C(O)OH, and C₁₋₆-alkyl-C(O) OC₁₋₆-alkyl; and
wherein *6 indicates the point of attachment to A⁶ and *4 indicates the point of attachment to A⁴;
A⁶ is 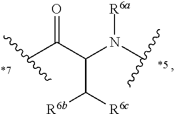
wherein:
R^{6a} is selected from H and C₁₋₆-alkyl;
R^{6b} is selected from H and C₁₋₆-alkyl;
R^{6c} is selected from C₁₋₆-alkyl and 5-10 membered heteroaryl, wherein the C₁₋₆-alkyl and 5-10 membered heteroaryl of R^{6c} are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —OC(O)—C₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *5 indicates the point of attachment to A⁵ and *7 indicates the point of attachment to A⁷;
A⁷ is 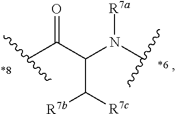
wherein:
R^{7a} is selected from H and C₁₋₆-alkyl;
R^{7b} is selected from H and C₁₋₆-alkyl;
R^{7c} is selected from C₁₋₆-alkyl and 5-10 membered heteroaryl, wherein the C₁₋₆-alkyl and 5-10 membered heteroaryl are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *8 indicates the point of attachment to A⁸ and *6 indicates the point of attachment to A⁶;
A⁸ is 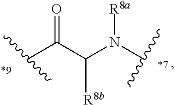
wherein:
R^{8a} is selected from H and C₁₋₆-alkyl;
R^{8b} is selected from H, C₁₋₆-alkyl, C₃₋₈-cycloalkyl, 5-7 membered heterocycloalkyl ring, and 5-10 membered heteroaryl, wherein the C₁₋₆-alkyl, C₃₋₈-cycloalkyl, 5-7 membered heterocycloalkyl ring, and 5-10 membered heteroaryl of R^{8b} are optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, and —OC₁₋₆-alkyl; and
wherein *9 indicates the point of attachment to A⁹ and *7 indicates the point of attachment to A⁷;
A⁹ is 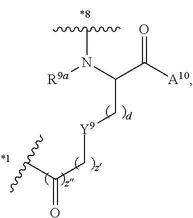
wherein:
Y⁹ is selected from a bond, C(O), NH, NC₁₋₆-alkyl, O, and S;
R^{9a} is selected from H and C₁₋₆-alkyl;
d is 1, 2, or 3; and
z′ and z″ are each independently 0 or 1; and
wherein *8 indicates the point of attachment to A⁸ and *1 indicates the point of attachment to A¹; and
A¹⁰ is 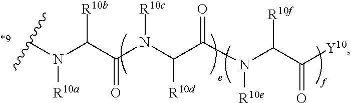
wherein:
Y¹⁰ is selected from OH and N(R^{10g})(R^{10h});
R^{10a}, R^{10c}, R^{10e}, R^{10g}, and R^{10h} are each independently selected from H and C₁₋₆-alkyl;
R^{10b} is selected from H, C₁₋₆-alkyl, —C₁₋₆-alkyl-phenyl, —C₁₋₆-alkyl-(5-6 membered heteroaryl), —C₁₋₇-alkyl-C(O)R¹⁰ⁱ, —C₁₋₆-alkyl-O—C(O)R¹⁰ⁱ, and —C₁₋₆-alkyl-NH—C(O)R¹⁰ⁱ, wherein the C₁₋₆-alkyl, —C₁₋₆-alkyl-phenyl, and —C₁₋₆-alkyl-(5-6 membered heteroaryl) of R¹⁰⁰ are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10a} and R^{10b}, together with the atoms to which they are attached, form a 5-7 membered heterocycloalkyl ring;
R^{10d} is selected from H, C₁₋₆-alkyl, —C₁₋₆-alkyl-phenyl, —C₁₋₆-alkyl-(5-6 membered heteroaryl), —C₁₋₇-alkyl-C(O)R^{10j}, —C₁₋₆-alkyl-O—C(O)R^{10j}, and —C₁₋₆-alkyl-NH—C(O)R^{10j}, wherein the C₁₋₆-alkyl, —C₁₋₆-alkyl-phenyl, and —C₁₋₆-alkyl-(5-6 membered heteroaryl) of R^{10d} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, —C₁₋₆-alkyl-(5-6 membered heteroaryl), —C₁₋₇-alkyl-C(O)R^{10k}, —C₁₋₆-alkyl-O—C(O)R^{10k}, and —C₁₋₆-alkyl-NH—C(O)R^{10k}, wherein the C₁₋₆-alkyl, —C₁₋₆-alkyl-phenyl, and —C₁₋₆-alkyl-(5-6 membered heteroaryl) of R^{10f} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10e} and R^{10f}, together with the atoms to which they are attached, form a 5-7 membered heterocycloalkyl ring;
R¹⁰ⁱ, R^{10j}, and R^{10k} are each independently selected from H, C₁₋₆-alkyl, C₃₋₇ cycloalkyl, 5-6 membered heteroaryl, and 3-7 membered heterocycloalkyl, wherein the C₃₋₇ cycloalkyl, 5-6 membered heteroaryl, and 3-7 membered heterocycloalkyl of R¹⁰ⁱ, R^{10j}, and R^{10k} are each optionally substituted with 1, 2, 3, or 4 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, C₁₋₆-alkyl, —C(O)OH, —C(O)NH₂, and C₁₋₆-alkyl-C(O)NH₂;
e and f are each independently 0 or 1; and
wherein *9 indicates the point of attachment to A⁹; and
b) at least one imaging agent, chelating agent, radionuclide, or cytotoxic drug, wherein at least one cyclized peptide \textcircled{P} is conjugated to the at least one imaging agent, chelating agent, radionuclide, or cytotoxic drug via any one of A¹-A¹⁰, optionally through a linker.

2. (canceled)

3. The compound of claim 1, or a pharmaceutically acceptable salt or solvate thereof, wherein the compound is a compound of formula (I-i): 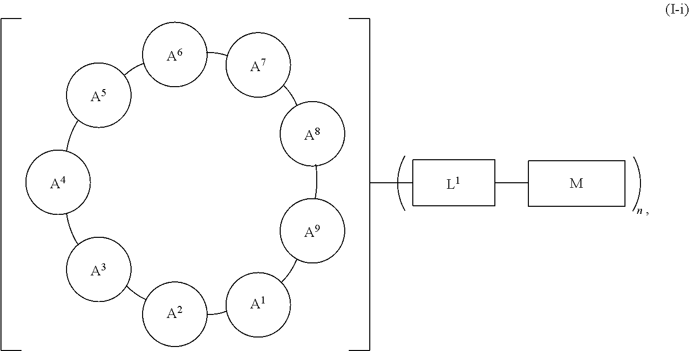
or a pharmaceutically acceptable salt or solvate thereof,
wherein:
L¹ is, independently at each occurrence, selected from a bond and a linker;
M is, independently at each occurrence, selected from an imaging agent, a chelating agent, and a radionuclide, wherein the chelating agent is optionally radiolabeled with a radionuclide;
n is 1, 2, 3, or 4; and
wherein any of A¹-A¹⁰ and L¹ are optionally substituted with an albumin binder or one or more PEG chains.

4. (canceled)

5. The compound of claim 1, or a pharmaceutically acceptable salt or solvate thereof, wherein:
R^{1a} is selected from H, C₁₋₆-alkyl, halo, —NH₂, —N(H)—C(O)—C₁₋₆-alkyl, —NHC₁₋₆-alkyl, and —N(C₁₋₆-alkyl)₂, wherein the C₁₋₆-alkyl, —N(H)—C(O)—C₁₋₆-alkyl, —NHC₁₋₆-alkyl, and —N(C₁₋₆-alkyl) 2 of R^{1a} are optionally substituted with 1 or 2 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, 5-membered heteroaryl, —OH, and —NH₂;
\textcircled{B} is selected from C₅₋₇-cycloalkyl and phenyl, wherein the C₅₋₇-cycloalkyl and phenyl of \textcircled{B} are optionally substituted with 1 substituent selected from C₁₋₆-alkyl, halo, —OH, and —NH₂;
R^{1aa} is selected from H, C₁₋₆-alkyl, —NHC₁₋₆-alkyl, —N(C₁₋₆-alkyl)₂, and —NH₂;
Y¹ is selected from a bond, C≡C, NH, NC₁₋₆-alkyl, O, and S;
Y^{1a} is selected from C(O), NH, NC₁₋₆-alkyl, C(O)NH, NHC(O), O, and S;
a is 1, 2, or 3;
b, c, t′, and x′ are each independently 0 or 1;
u′ is 0, 1, or 2;
R^{2a} is selected from H and C₁₋₃-alkyl;
R^{2b} is selected from H, C₁₋₆-alkyl, and halo;
R^{2c} is selected from C₁₋₆-alkyl, phenyl, and 6-membered heteroaryl, wherein the phenyl and 6-membered heteroaryl of R^{2c} are optionally substituted with 1 or 2 substituents independently selected from halo, —C(O)NH₂, —C(O)OH, —C(O)C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, and —NH₂;
R^{2d} is selected from H, C₁₋₆-alkyl, and halo;
R^{3a} is selected from H and C₁₋₃-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-(6-membered heterocycloalkyl), —C₁₋₆-alkyl-N(H)-(6-membered heterocycloalkyl), —C₁₋₆-alkyl-N(H)—C(O)—(6-membered heterocycloalkyl), and —C₁₋₆-alkyl-N(H)—C₁₋₆-alkyl-(6-membered heterocycloalkyl),
wherein the C₁₋₆-alkyl, —C₁₋₆-alkyl-C(O)NH—C₁₋₆-alkyl, —C₁₋₆-alkyl-(6-membered heterocycloalkyl), —C₁₋₆-alkyl-N(H)-(6-membered heterocycloalkyl), —C₁₋₆-alkyl-N(H)—C(O)—(6-membered heterocycloalkyl), and —C₁₋₆-alkyl-N(H)—C₁₋₆-alkyl-(6-membered heterocycloalkyl) of R^{3c} are optionally substituted with 1 2, 3, 4, or 5 substituents independently selected from —C(O)OH, —C₁₋₆-alkyl-C(O)OH, —C(O)NH₂, —NH₂, —NHC(O)C₁₋₆-alkyl, —OH, and —OC₁₋₆-alkyl;
R^{4a} is selected from H and C₁₋₃-alkyl, wherein the C₁₋₃-alkyl of R^{4a} is optionally substituted with 1 substituent selected from —NH₂, —OH, —OC₁₋₆-alkyl, and C₁₋₆-alkyl;
R^{4b} is selected from H, C₁₋₆-alkyl, —C₁₋₆-alkyl-phenyl, —C₁₋₆-alkyl-pyridyl, and —C₁₋₆-alkyl-NH—C(O)OH, wherein the C₁₋₆-alkyl, —C₁₋₆-alkyl-phenyl, and —C₁₋₆-alkyl-pyridyl of R^{4b} are optionally substituted with 1 or 2 substituents independently selected from halo, —NH₂, —OH, and —OC₁₋₆-alkyl;
R^{5a} is selected from H and C₁₋₃-alkyl, wherein the C₁₋₃-alkyl of R^{5a} is optionally substituted with 1 substituent selected from —NH₂, —NHC₁₋₆-alkyl, —N(C₁₋₆-alkyl)₂, and —OH;
R^{5b} is selected from H, C₁₋₆-alkyl, —C₁₋₆-alkyl-O—C(O)R^{5c}, and —C₁₋₆-alkyl-NH—C(O)R^{5c}, wherein the C₁₋₆-alkyl, —C₁₋₆-alkyl-O—C(O)R^{5c}, and —C₁₋₆-alkyl-NH—C(O)R^{5c} of R^{5b} are each optionally substituted with 1 or 2 substituents independently selected from —NH₂, —NHC₁₋₆-alkyl, —N(C₁₋₆-alkyl)₂, and —OH;
R^{5c} is selected from H, C₁₋₆-alkyl, —NH₂, —OH, —OC₁₋₆-alkyl, C₁₋₆-haloalkyl, C₁₋₆-alkyl-C(O)OH, and C₁₋₆-alkyl-C(O) OC₁₋₆-alkyl;
R^{6a} is selected from H and C₁₋₃-alkyl;
R^{6b} is selected from H and C₁₋₆-alkyl;
R^{6c} is selected from C₁₋₆-alkyl and 8-9 membered heteroaryl, wherein the C₁₋₆-alkyl and 8-9 membered heteroaryl of R^{6c} are each optionally substituted with 1 or 2 substituents independently selected from C₁₋₆-alkyl, —CN, halo, —NH₂, —OH, —OC₁₋₆-alkyl, and —OC(O)—C₁₋₆-alkyl;
R^{7a} is selected from H and C₁₋₃-alkyl;
R^{7b} is selected from H and C₁₋₆-alkyl;
R^{7c} is selected from C₁₋₆-alkyl and 8-9 membered heteroaryl, wherein the C₁₋₆-alkyl and 8-9 membered heteroaryl are each optionally substituted with 1 or 2 substituents independently selected from —CN, halo, —NH₂, and —OH;
R^{8a} is selected from H and C₁₋₃-alkyl;
R^{8b} is selected from H, C₁₋₆-alkyl, and C₃₋₆-cycloalkyl, wherein the C₁₋₆-alkyl and C₃₋₆-cycloalkyl of R^{8b} are optionally substituted with 1 or 2 substituents independently selected from —CN, halo, —NH₂, and —OH;
Y⁹ is selected from a bond, C(O), NH, NC₁₋₆-alkyl, O, and S;
R^{9a} is selected from H and C₁₋₃-alkyl;
d is 1 or 2;
Y¹⁰ is selected from OH and N(R^{10g})(R^{10h});
R^{10a}, R^{10c}, and R^{10e} are each independently selected from H and C₁₋₃-alkyl;
R^{10g} and R^{10h} are each independently selected from H and C₁₋₆-alkyl;
R^{10b} is selected from H, C₁₋₃-alkyl, —C₁₋₆-alkyl-phenyl, and —C₁₋₆-alkyl-NH—C(O)R¹⁰ⁱ, wherein the C₁₋₆-alkyl and —C₁₋₆-alkyl-phenyl of R^{10b} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
R^{10d} is selected from H, C₁₋₆-alkyl, —C₁₋₆-alkyl-phenyl, and —C₁₋₆-alkyl-NH—C(O)R^{10j}, wherein the C₁₋₆-alkyl and —C₁₋₆-alkyl-phenyl of R^{10d} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, and —C₁₋₆-alkyl-NH—C(O)R^{10k}, wherein the C₁₋₆-alkyl, —C₁₋₆-alkyl-phenyl, and —C₁₋₆-alkyl-(5-6 membered heteroaryl) of R^{10f} are optionally substituted with 1 or 2 substituents independently selected from halo, —NH₂, —N₃, —OH, —OC₁₋₆-alkyl, C₁₋₆-alkyl, —C(O)NH₂, —C(O)OH, and —C₁₋₆-alkyl-C(O)OH;
R¹⁰ⁱ, R^{10j}, and R^{10k} are each independently selected from H, C₁₋₆-alkyl, 6-membered heterocycloalkyl, and 6-membered heteroaryl, wherein the 6-membered heterocycloalkyl, and 6-membered heteroaryl of R¹⁰ⁱ, R^{10j}, and R^{10k} are each optionally substituted with 1 or 2 substituents independently selected from halo, —NH₂, —NHC₁₋₆-alkyl, —N(C₁₋₆-alkyl)₂, —OH, C₁₋₆-alkyl, and C₁₋₆-alkyl-C(O)OH; and
e and f are each independently 0 or 1.

6. The compound of claim 1, or a pharmaceutically acceptable salt or solvate thereof, wherein:
A¹ is a moiety of formula (A1-I), formula (A1-II), formula (A1-III), or formula (A1-IV):

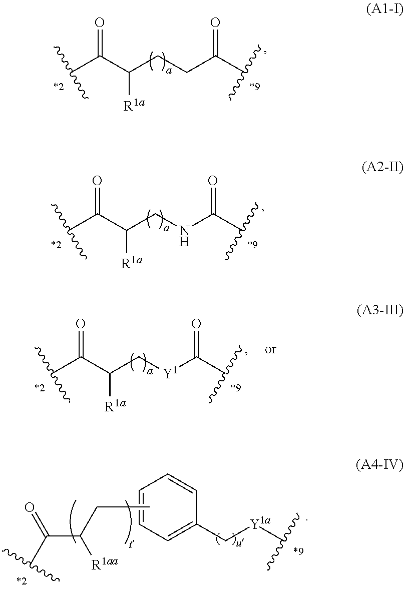

7. The compound of claim 1, or a pharmaceutically acceptable salt or solvate thereof, wherein A¹ is selected from:

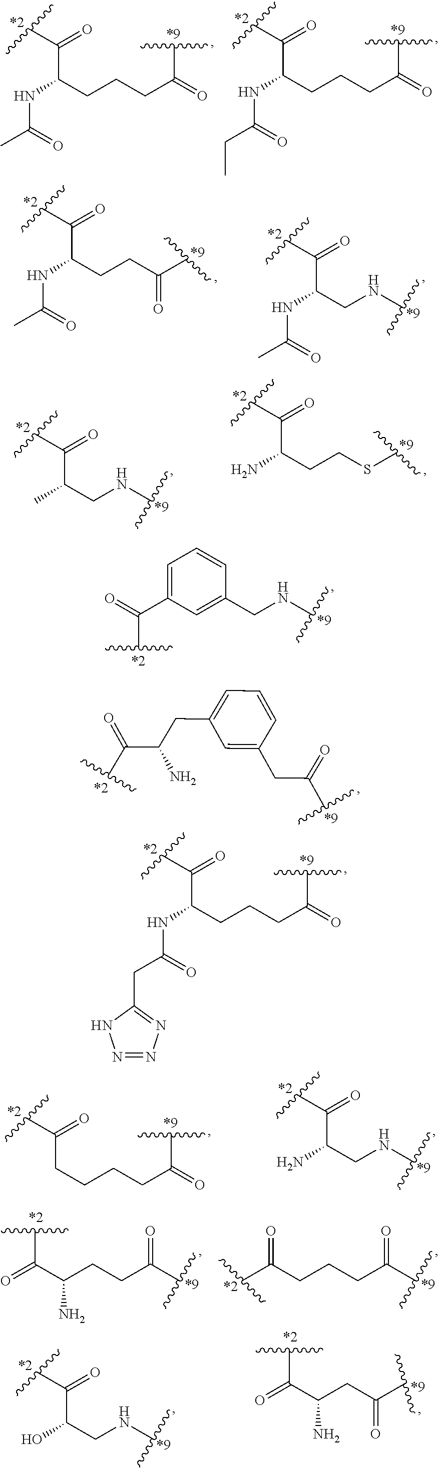

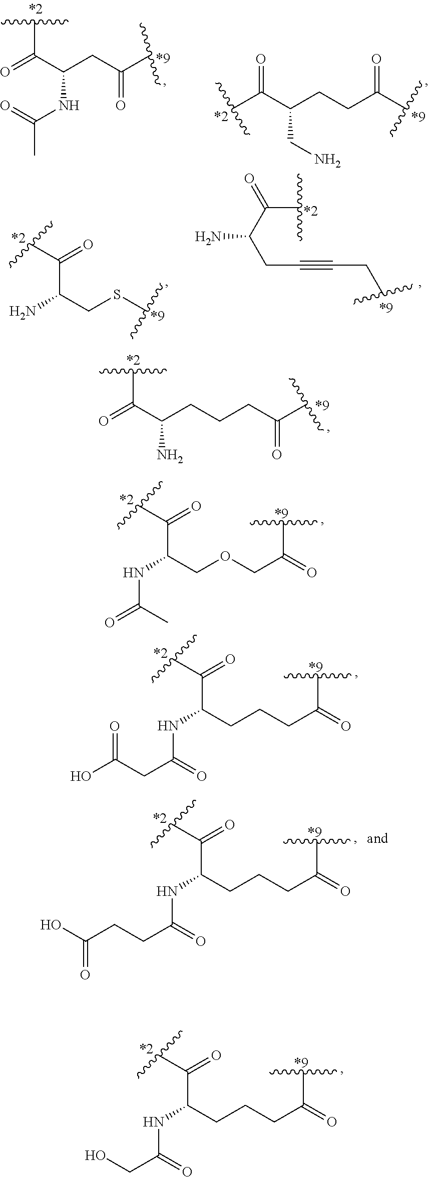

8. The compound of claim 1, or a pharmaceutically acceptable salt or solvate thereof, wherein:
A² is a moiety of formula (A2-I): 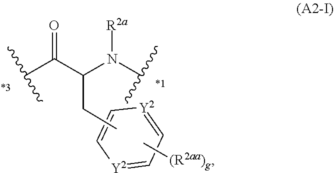
wherein:
each Y² is independently selected from N and CH;
R^{2a} is selected from H and C₁₋₃-alkyl;
each R^{2a} is independently selected from halo, —C(O)NH₂, —C(O)OH, —C(O)C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, and —NH₂; and
g is 0, 1, or 2.

9. The compound of claim 1, or a pharmaceutically acceptable salt or solvate thereof, wherein A² is selected from:

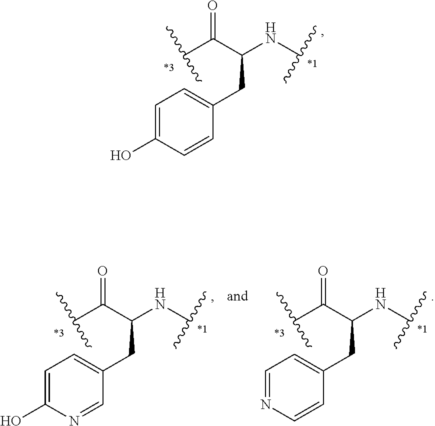 [SMILES: *N[C@@H](Cc1ccc(O)cc1)C(*)=O ; *N[C@@H](Cc1ccc(O)nc1)C(*)=O ; *N[C@@H](Cc1ccncc1)C(*)=O]

10. The compound of claim 1, or a pharmaceutically acceptable salt or solvate, thereof, wherein:
A³ is a moiety of formula (A3-I) or formula (A3-II): 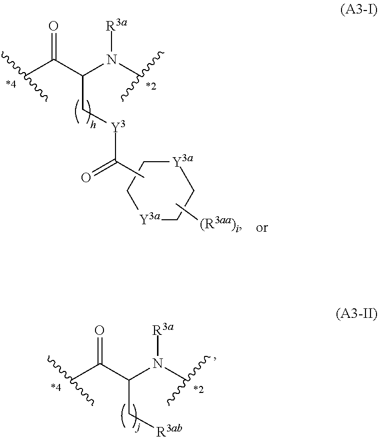
wherein:
Y³ is selected from a bond and NH;
each Y^{3a} is selected from NH and CH₂, provided that at least one Y^{3a} is NH;
R^{3a} is selected from H and C₁₋₃-alkyl;
each R^{3aa} is independently selected from —C(O)OH, —C₁₋₆-alkyl-C(O)OH, —C(O)NH₂, —NH₂, —NHC(O)C₁₋₆-alkyl, and —OC₁₋₆-alkyl;
R^{3ab} is selected from H, —OH, —C(O)OH, —C₁₋₆-alkyl-C(O)OH, —C(O)NH₂, —NH₂, —NHC(O)C₁-6-alkyl, —C(O)NHC₁₋₆-alkyl, and —OC₁₋₆-alkyl, wherein the —C(O)NHC₁₋₆-alkyl of R^{3ab} is optionally substituted with 1, 2, 3, 4, or 5 substituents independently selected from halo and —OH;
h is 1, 2, 3, 4, 5, or 6;
i is 0, 1, or 2; and
j is 1, 2, 3, 4, 5, or 6.

11. The compound of claim 1, or a pharmaceutically acceptable salt or solvate thereof, wherein A³ is selected from:

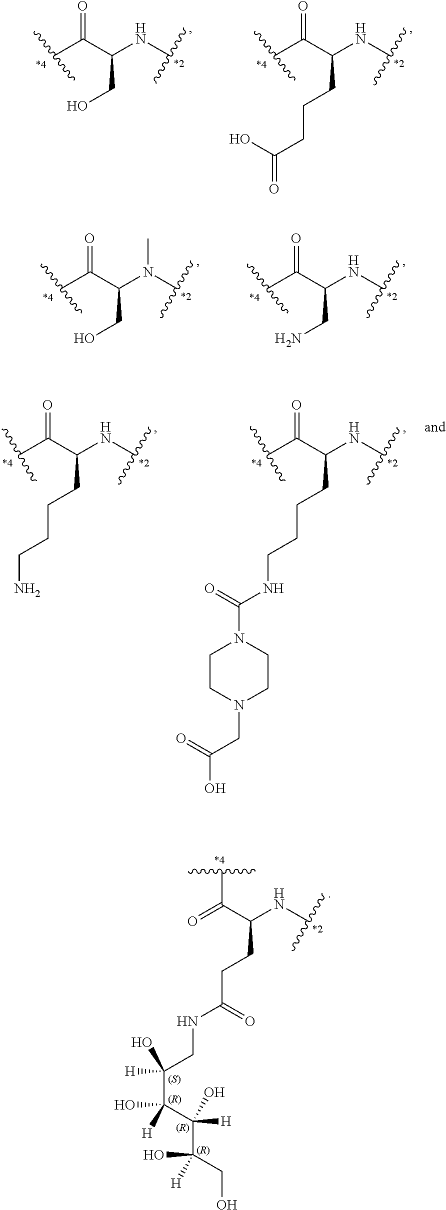

12. The compound of claim 1, or a pharmaceutically acceptable salt or solvate thereof, wherein:
A⁴ is a moiety of formula (A4-I) or formula (A4-II): 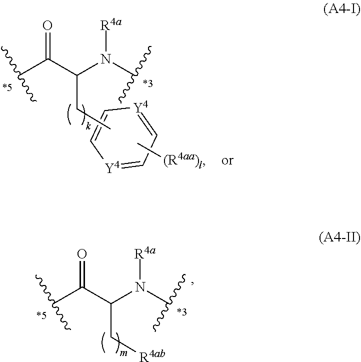
wherein:
each Y⁴ is independently selected from N and CH;
R^{4a} is selected from H and C₁₋₃-alkyl;
each R^{4a} is independently selected from C₁₋₆-alkyl, halo, —NH₂, —OH, and —OC₁₋₆-alkyl;
R^{4ab} is selected from H, halo, —NH₂, —OH, —OC₁₋₆-alkyl, and —O-phenyl;
k is 1, 2, 3, 4, 5, or 6;
l is 0, 1, or 2; and
m is 1, 2, 3, 4, 5, or 6.

13. The compound of claim 1, or a pharmaceutically acceptable salt or solvate thereof, wherein A⁴ is selected from:

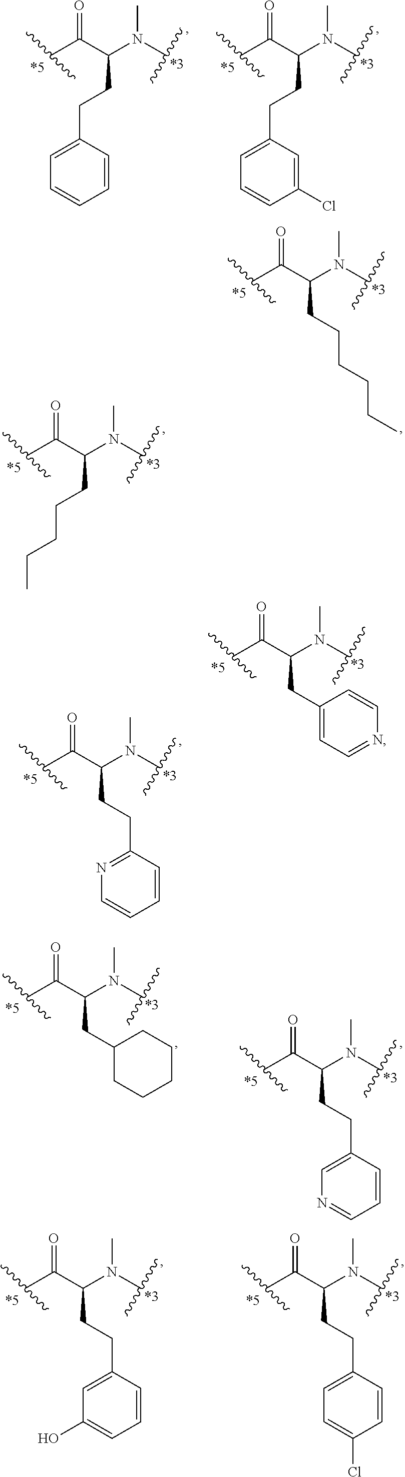

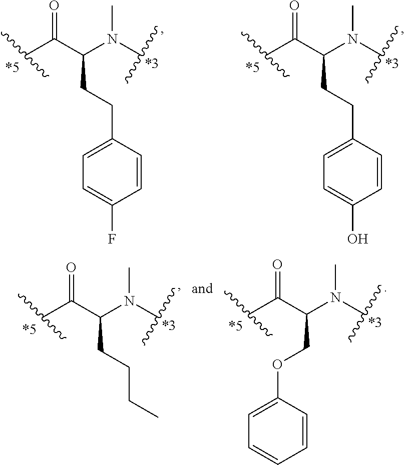

14. The compound of claim 1, or a pharmaceutically acceptable salt or solvate thereof, wherein:
A⁵ is a moiety of formula (A5-1), formula (A5-II), or formula (A5-III): 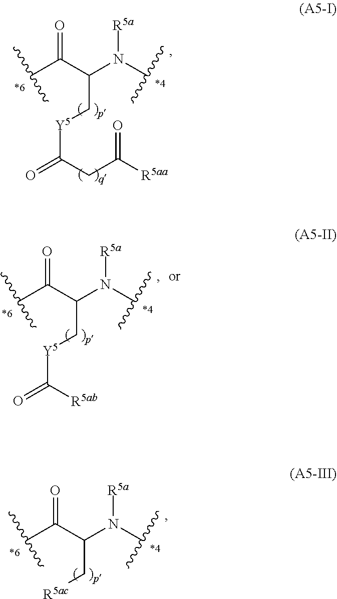
wherein:
Y⁵ is selected from O and NH;
R^{5a} is selected from H and C₁₋₃-alkyl;
R^{5aa} is selected from H, —OH, and —OC₁₋₆-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
R^{5ac} is selected from H, —NH₂, —NHC₁₋₆-alkyl, —N(C₁₋₆-alkyl)₂, and —OH; and
p′ and q′ are each independently 1, 2, 3, 4, 5, or 6.

15. The compound of claim 1, or a pharmaceutically acceptable salt or solvate thereof, wherein A⁵ is selected from:

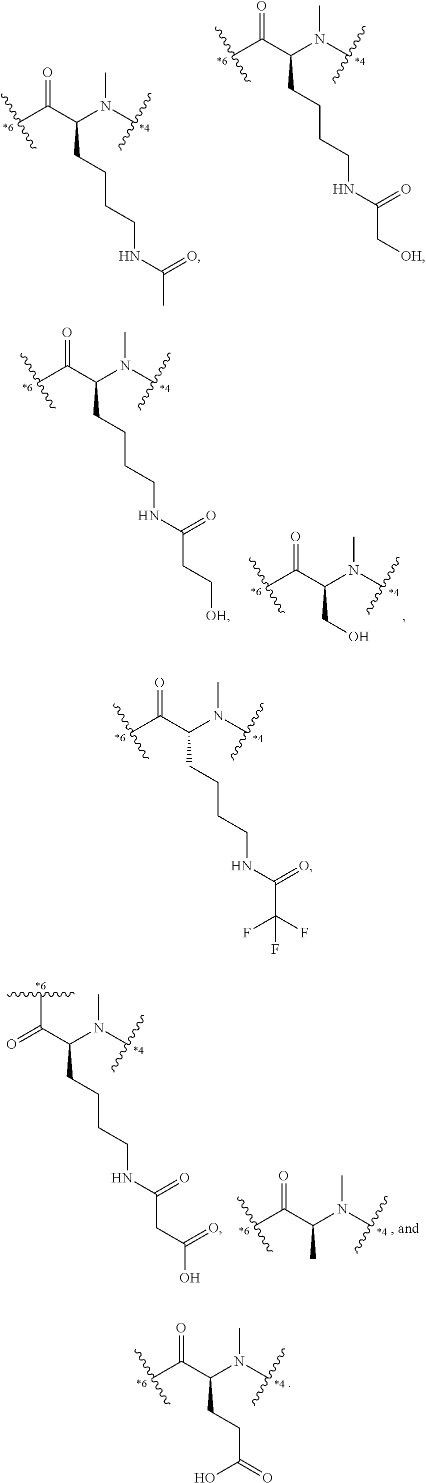

16-17. (canceled)

18. The compound of claim 1, or a pharmaceutically acceptable salt or solvate thereof, wherein:
A⁶ is a moiety of formula (A6-I), formula (A6-II), or formula (A6-III): 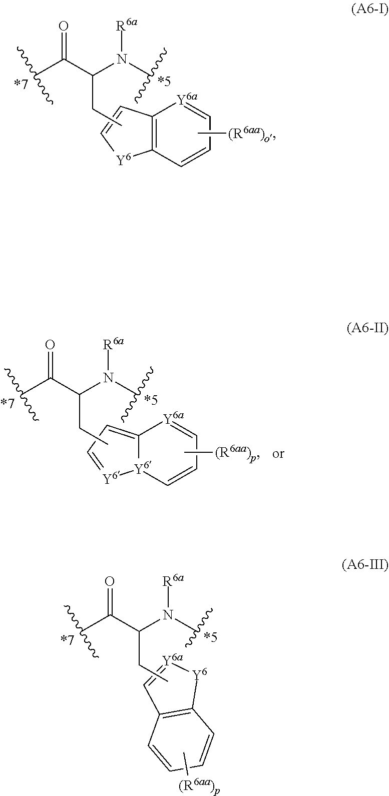
wherein:
Y⁶ is selected from NH and CH₂;
Y^{6a} and Y^{6′} are each independently selected from N and CH, provided that at least one of Y⁶ and Y^{6a} is selected from N and NH and provided that at least one of Y^{6′} and Y^{6a} is N;
R^{6a} is selected from H and C₁₋₃-alkyl;
each R^{6aa} is selected from C₁₋₆-alkyl, —CN, halo, —NH₂, —OH, —OC₁₋₆-alkyl, and —OC(O)—C₁₋₆-alkyl; and
o′ and p are each independently 0, 1, or 2.

19. (canceled)

20. The compound of claim 1, or a pharmaceutically acceptable salt or solvate thereof, wherein A⁶ is selected from:

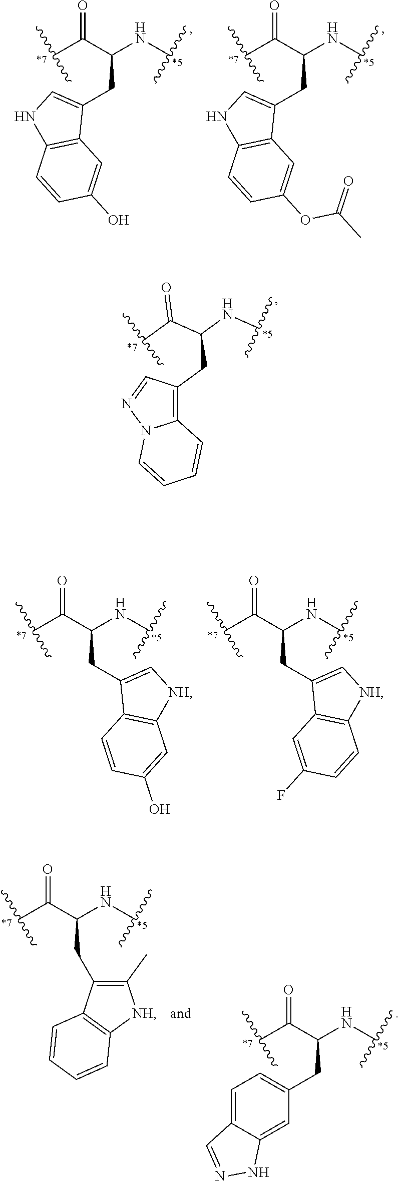

21-22. (canceled)

23. The compound of claim 1, or a pharmaceutically acceptable salt or solvate thereof, wherein:
A⁷ is a moiety of formula (A7-I) or formula (A7-II): 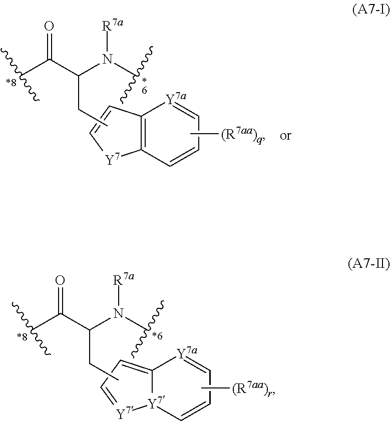
wherein:
Y⁷ is selected from NH and CH₂;
Y⁷a and YT are each independently selected from N and CH, provided that at least one of Y⁷ and Y^{7a} is selected from N and NH and provided that at least one of Y″ and Y⁷a is N;
R^{7a} is selected from H and C₁₋₃-alkyl;
each R^{7aa} is selected from C₁₋₃-alkyl, —CN, halo, —NH₂, and —OH; and
q and r are each independently 0, 1, or 2.

24. (canceled)

25. The compound of claim 1, or a pharmaceutically acceptable salt or solvate thereof, wherein A⁷ is selected from:

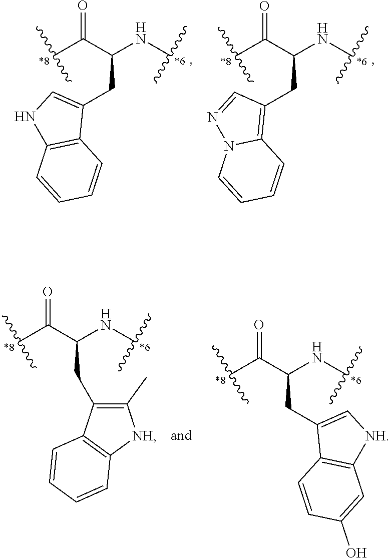 [SMILES: *N[C@@H](Cc1c[nH]c2ccccc12)C(*)=O ; *N[C@@H](Cc1cnn2ccccc12)C(*)=O ; *N[C@@H](Cc1c(C)[nH]c2ccccc12)C(*)=O ; *N[C@@H](Cc1c[nH]c2cc(O)ccc12)C(*)=O]

26. The compound of claim 1, or a pharmaceutically acceptable salt or solvate thereof, wherein:
A⁸ is a moiety of formula (A8-I) or formula (A8-II): 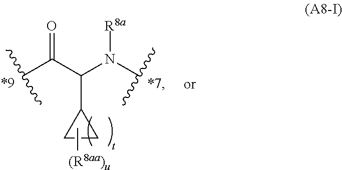 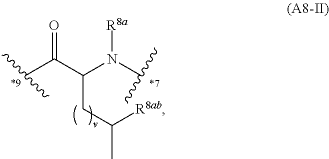
wherein:
R^{8a} is selected from H and C₁₋₃-alkyl;
R^{8aa} is selected from —CN, halo, —NH₂, and —OH;
R^{8ab} is selected from H, —CN, halo, —NH₂, and —OH;
t is 1 or 2;
u is 0, 1, or 2; and
v is 0, 1, 2, 3, or 4.

27. The compound of claim 1, or a pharmaceutically acceptable salt or solvate thereof, wherein A⁸ is selected from:

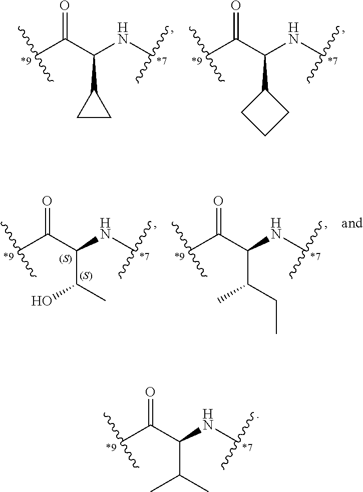

28. The compound of claim 1, or a pharmaceutically acceptable salt or solvate thereof, wherein:
A⁹ is a moiety of formula (A9-1) or formula (A9-II): 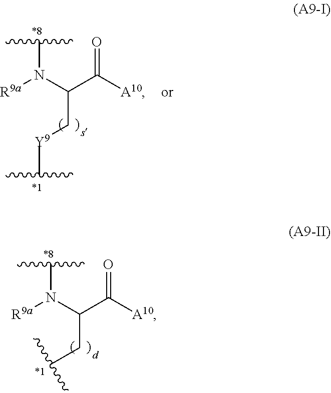
wherein:
Y⁹ is selected from C(O), NH, NC₁₋₆-alkyl, and S;
R^{9a} is selected from H and C₁₋₃-alkyl; and
s′ is 1 or 2; and
d is 1 or 2.

29. The compound of claim 1, or a pharmaceutically acceptable salt or solvate thereof, wherein A⁹ is selected from:

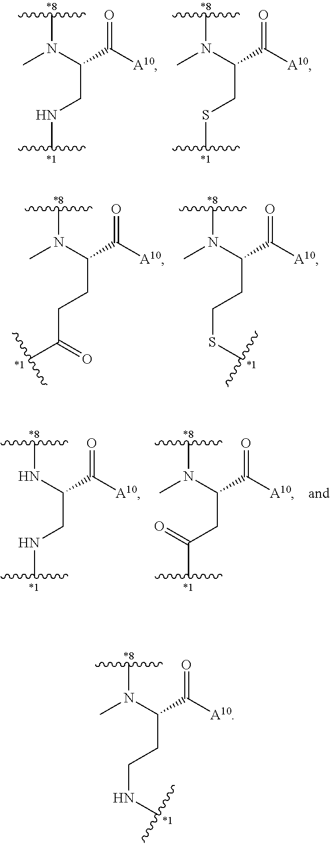

30. The compound of claim 1, or a pharmaceutically acceptable salt or solvate thereof, wherein:
A¹⁰ is a moiety of formula (A10-I), formula (A10-II), formula (A10-III), or formula (A10-IV):

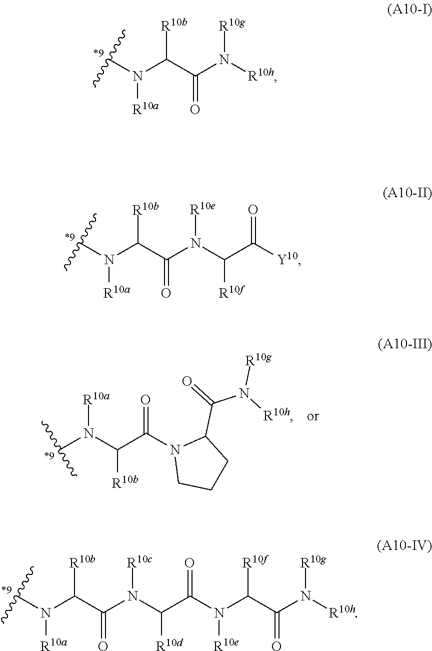

31-34. (canceled)

35. The compound of claim 1, or a pharmaceutically acceptable salt or solvate thereof, wherein A¹⁰ is selected from:

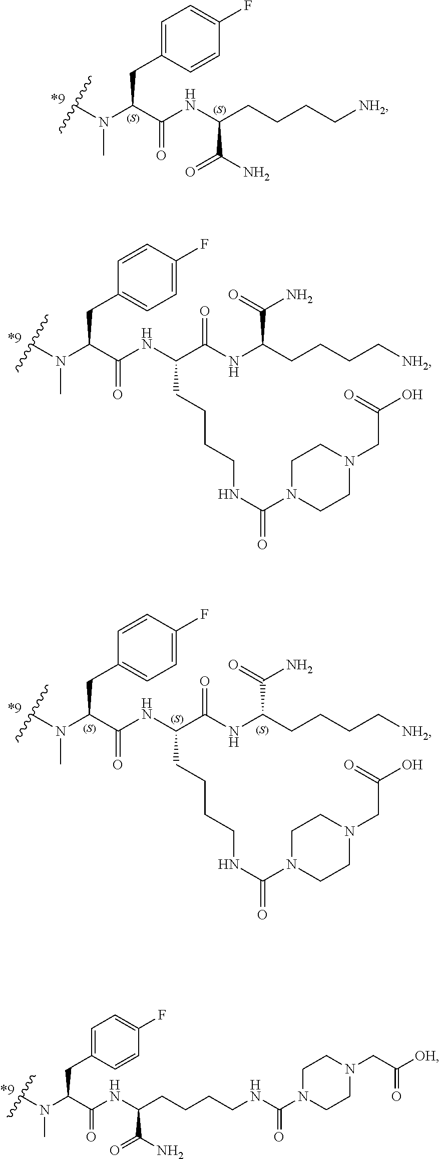

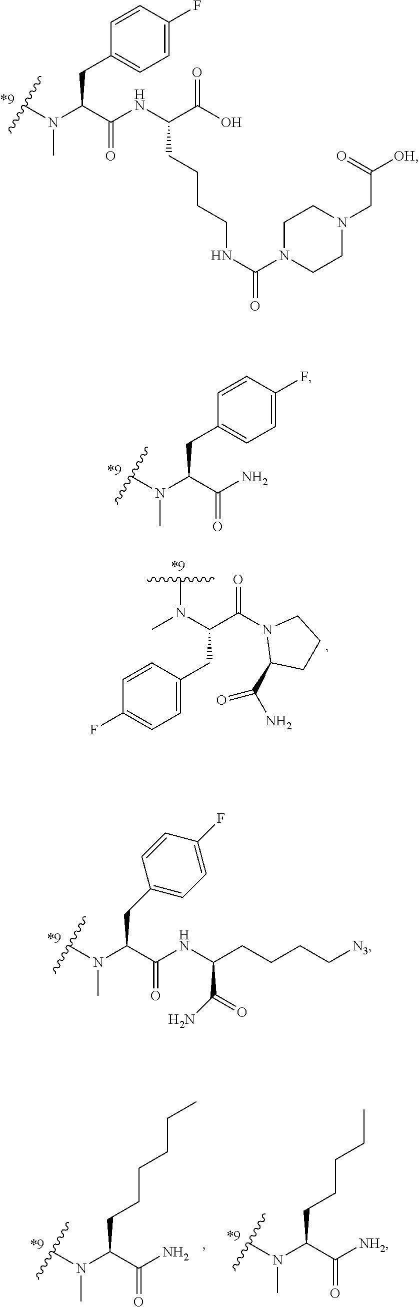 [SMILES: *N(C)[C@@H](Cc1ccc(F)cc1)C(=O)N[C@@H](CCCCNC(=O)N1CCN(CC(=O)O)CC1)C(=O)O ; *N(C)[C@@H](Cc1ccc(F)cc1)C(N)=O ; *N(C)[C@@H](Cc1ccc(F)cc1)C(=O)N1CCC[C@H]1C(N)=O ; *N(C)[C@@H](Cc1ccc(F)cc1)C(=O)N[C@@H](CCCCN=[N+]=[N-])C(N)=O ; *N(C)[C@@H](CCCCCC)C(N)=O ; *N(C)[C@@H](CCCCC)C(N)=O]

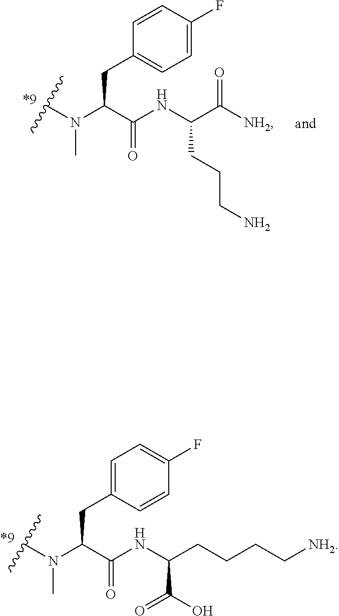 [SMILES: *N(C)[C@@H](Cc1ccc(F)cc1)C(=O)N[C@@H](CCCN)C(N)=O ; *N(C)[C@@H](Cc1ccc(F)cc1)C(=O)N[C@@H](CCCCN)C(=O)O]

36-43. (canceled)

44. The compound of claim 3, or a pharmaceutically acceptable salt or solvate thereof, wherein M is a cyclic chelating agent.

45-49. (canceled)

50. The compound of claim 3, or a pharmaceutically acceptable salt or solvate thereof, wherein L¹ is a bond, and A¹⁰, L¹, and M, together, have the structure: 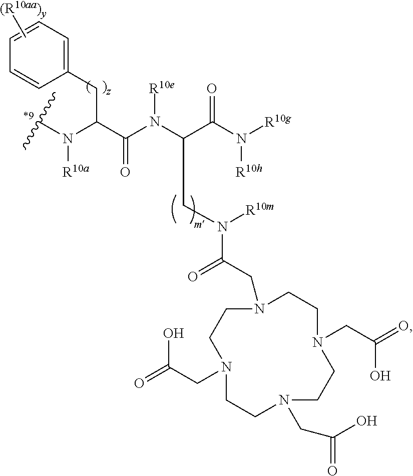
wherein:
R^{10m} is selected from H and C₁₋₃-alkyl; and
m′ is 1, 2, 3, 4, 5, or 6,
wherein M is optionally radiolabeled with a radionuclide.

51-54. (canceled)

55. The compound of claim 3, or a pharmaceutically acceptable salt or solvate thereof, wherein the compound has a structure of formula (I), wherein:
A¹ is a moiety of formula (A1-I): 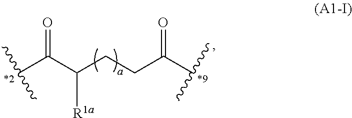
wherein:
R^{1a} is selected from H, C₁₋₆-alkyl, halo, —NH₂, —N(H)—C(O)—(C₁₋₆-alkyl), —NHC₁₋₆-alkyl, and —N(C₁₋₆-alkyl)₂, wherein the C₁₋₆-alkyl and —N(H)—C(O)—C₁₋₆-alkyl of R^{1a} are optionally substituted with 1 substituent selected from halo, —C(O)NH₂, —C(O)OH, —C(O)C₁₋₆-alkyl, —C(O)C₁₋₆-alkyl-OH, and 5-membered heteroaryl; and
a is 1, 2, or 3;
A² is a moiety of formula (A2-I): 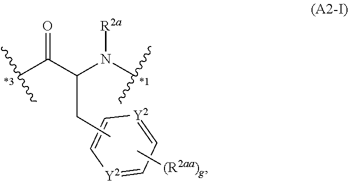
wherein:
each Y² is independently selected from N and CH;
R^{2a} is selected from H and C₁₋₃-alkyl;
each R^{2aa} is independently selected from halo, —C(O)NH₂, —C(O)OH, —C(O)C₁₋₆-alkyl, —OH, and —NH₂; and
g is 0, 1, or 2;
A³ is a moiety of formula (A3-II): 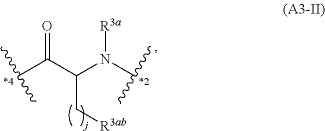
wherein:
R^{3a} is selected from H and C₁₋₃-alkyl;
R^{3ab} is selected from H, —OH, —C(O)OH, —C₁₋₆-alkyl-C(O)OH, —C(O)NH₂, —NH₂, —NHC(O)C₁₋₆-alkyl, and —OC₁₋₆-alkyl; and
j is 1, 2, 3, 4, 5, or 6;
A⁴ is a moiety of formula (A4-I): 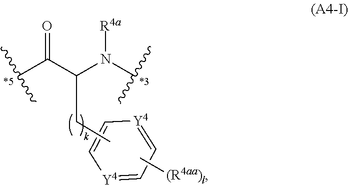
wherein:
each Y⁴ is independently selected from N and CH;
R^{4a} is selected from H and C₁₋₃-alkyl;
each R^{4aa} is independently selected from C₁₋₆-alkyl, halo, —NH₂, —OH, and —OC₁₋₆-alkyl;
k is 1, 2, 3, 4, 5, or 6; and
l is 0, 1, or 2;
A⁵ is a moiety of formula (A5-II): 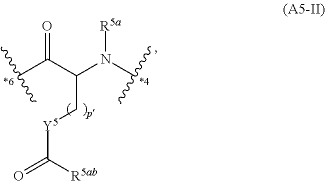
wherein:
Y⁵ is selected from O and NH;
R^{5a} is selected from H and C₁₋₃-alkyl;
R^{5ab} is selected from H, C₁₋₆-alkyl, —NH₂, —OH, and C₁₋₆-haloalkyl, wherein the C₁₋₆-alkyl of R^{5ab} is optionally substituted with 1 substituent independently selected from —NH₂ and —OH;
p′ is 1, 2, 3, 4, 5, or 6;
A⁶ is a moiety of formula (A6-la): 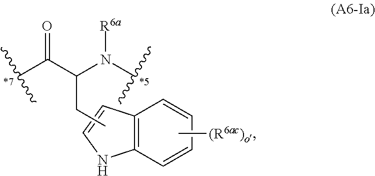
wherein:
R^{6a} is selected from H and C₁₋₃-alkyl;
R^{6ac} is selected from OH, C₁₋₆-alkyl, and —C(O)—C₁₋₆-alkyl; and
o′ is 0, 1, or 2;
A⁷ is a moisty of formula (A7-la): 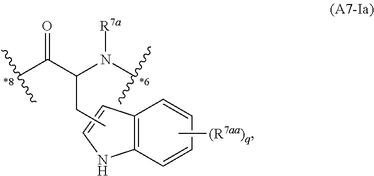
wherein:
R^{7a} is selected from H and C₁₋₃-alkyl;
each R^{7aa} is selected from —CN, halo, —NH₂, and —OH; and
q is 0, 1, or 2;
A⁸ is a moiety of formula (A8-I): 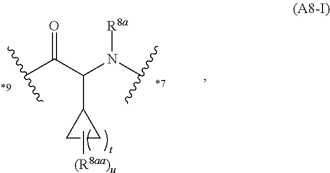
wherein:
R^{8a} is selected from H and C₁₋₃-alkyl;
R^{8aa} is selected from —CN, halo, —NH₂, and —OH;
t is 1 or 2; and
u is 0, 1, or 2;
A⁹ is a moiety of formula (A9-I): 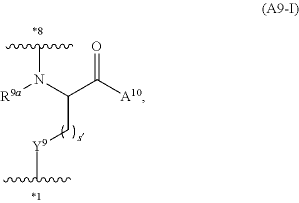
wherein:
Y⁹ is selected from C(O), NH, NC₁₋₄-alkyl, and S;
R^{9a} is selected from H and C₁₋₃-alkyl; and
s′ is 1 or 2; and
A¹⁰, L¹, and M, together, have the structure: 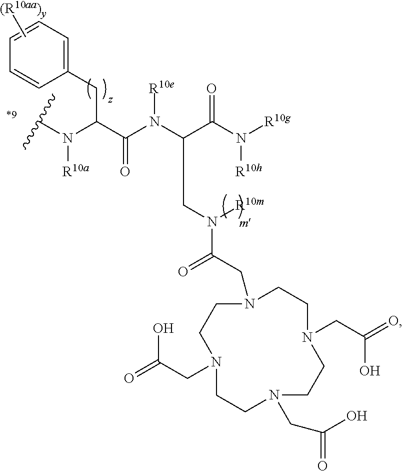
wherein:
R^{10a}, R^{10e}, and R^{10m} are each independently selected from H and C₁₋₃-alkyl;
R^{10g} and R^{10h} are each independently selected from H and C₁₋₆-alkyl;
each R^{10aa} is independently selected from C₁₋₆-alkyl, halo, —NH₂, —N₃, —OH, and —OC₁₋₆-alkyl;
y is 0, 1, or 2;
z is 1, 2, 3, 4, 5, or 6; and
m′ is 1, 2, 3, 4, 5, or 6,
wherein M is optionally radiolabeled with a radionuclide.

56. The compound of claim 1, which is: 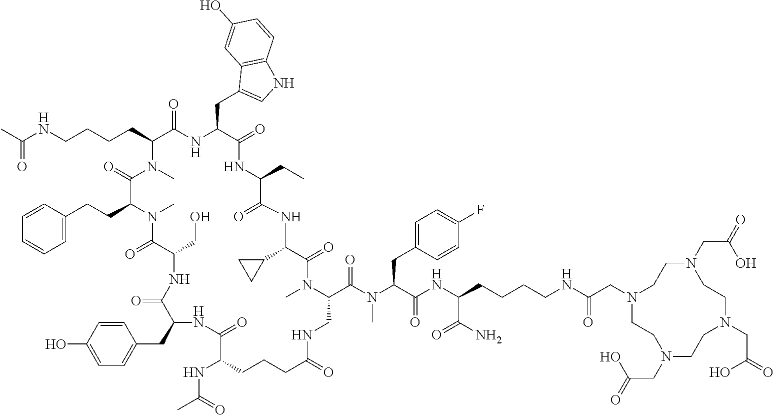 [SMILES: CC[C@@H]1NC(=O)[C@H](Cc2c[nH]c3ccc(O)cc23)NC(=O)[C@H](CCCCNC(C)=O)N(C)C(=O)[C@H](CCc2ccccc2)N(C)C(=O)[C@H](CO)NC(=O)[C@H](Cc2ccc(O)cc2)NC(=O)[C@@H](NC(C)=O)CCCC(=O)NC[C@@H](C(=O)N(C)[C@@H](Cc2ccc(F)cc2)C(=O)N[C@@H](CCCCNC(=O)CN2CCN(CC(=O)O)CCN(CC(=O)O)CCN(CC(=O)O)CC2)C(N)=O)N(C)C(=O)[C@H](C2CC2)NC1=O]
or a pharmaceutically acceptable salt or solvate thereof, which is optionally radiolabeled with a radionuclide.

57. (canceled)

58. The compound of claim 1, which is radiolabeled with a radionuclide.

59. The compound of claim 1 as selected from B1-B8, C1-C30, D1-D6, E1-E3, F1-F4, and G1, or a pharmaceutically acceptable salt or solvate thereof, which is optionally radiolabeled with a radionuclide.

60. (canceled)

61. A pharmaceutical composition comprising a compound of claim 1, or a pharmaceutically acceptable salt or solvate thereof, and one or more pharmaceutically acceptable carriers.

62. A pharmaceutical composition comprising a compound of claim 58, or a pharmaceutically acceptable salt or solvate thereof, and one or more pharmaceutically acceptable stabilizers.

63-64. (canceled)

65. A method of imaging cancer in a subject, comprising administering to the subject a compound according to claim 58, or a pharmaceutically acceptable salt or solvate thereof.

66-67. (canceled)

68. A method of treating cancer in a subject, comprising administering to the subject a therapeutically effective amount of a compound according to claim 58.

69-72. (canceled)

73. A cyclized peptide (P): 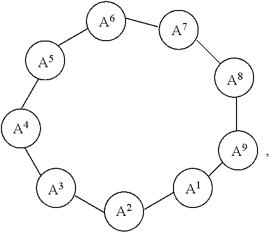
or a pharmaceutically acceptable salt or solvate thereof,
wherein:
A¹ is 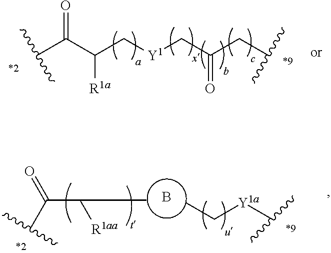
wherein:
R^{1a} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁-6-alkyl)₂, and —NH₂;
\textcircled{B} is selected from C₃₋₈-cycloalkyl, phenyl, and 5-6 membered heteroaryl, wherein the C₃₋₈-cycloalkyl, phenyl, and 5-6 membered heteroaryl of \textcircled{B} are optionally substituted with 1 or 2 substituents independently selected from halo, —OH, —NH₂, and C₁₋₆-alkyl;
R^{1aa} is selected from H, C₁₋₆-alkyl, OH, halo, —NH₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —NHC₁₋₆-alkyl, —N(C₁₋₆-alkyl)₂, C₃₋₈-cycloalkyl, phenyl, and 5-6 membered heteroaryl, wherein the C₁₋₆-alkyl, —N(H)—C(O)—C₁₋₆-alkyl, —NHC₁₋₆-alkyl, —N(C₁₋₆-alkyl)₂, phenyl, and 5-6 membered heteroaryl of R^{1a} are optionally substituted with 1, 2, 3, or 4 substituents independently selected from halo, —C(O)NH₂, —C(O)OH, —C(O)C₁₋₆-alkyl, —C(O)C₁₋₆-alkyl-OH, —C(O)C₁₋₆-alkyl-C(O)OH, —C(O)C₁₋₆-alkyl-NH₂, —C(O)C₁₋₆-alkyl-NHC₁₋₆-alkyl, —C(O)C₁₋₆-alkyl-N(C₁₋₆-alkyl)₂, 5-6 membered heteroaryl, —OH, —NHC₁₋₆-alkyl, —N(C₁-6-alkyl)₂, and —NH₂;
Y¹ is selected from a bond, C≡C, NH, NC₁₋₆-alkyl, O, and S;
Y¹a is selected from C(O), NH, NC₁₋₆-alkyl, C(O)NH, C(O)NC₁₋₆-alkyl, NHC(O), N(C₁₋₆-alkyl) C(O), O, and S;
a is 1, 2, 3, or 4;
b, c, t′, and x′ are each independently 0 or 1;
u′ is 0, 1, 2, or 3; and
*2 indicates the point of attachment to A² and *9 indicates the point of attachment to A⁹;
A² is 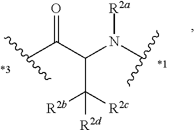
wherein:
R^{2a} is selected from H and C₁₋₆-alkyl;
R^{2b} is selected from H, C₁₋₆-alkyl, and halo;
R^{2c} is selected from halo, C₁₋₆-alkyl, C₃₋₈-cycloalkyl, phenyl, and 5-6 membered heteroaryl, wherein the C₁₋₆-alkyl, phenyl, and 5-6 membered heteroaryl of R^{2c} are optionally substituted with 1, 2, 3, or 4 substituents independently selected from halo, —C(O)NH₂, —C(O)OH, —C(O)C₁₋₆-alkyl, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —NHC₁₋₆-alkyl, —N(C₁₋₆-alkyl)₂, and —NH₂;
R^{2d} is selected from H, C₁₋₆-alkyl, and halo; and
*3 indicates the point of attachment to A³ and *1 indicates the point of attachment to A1;
A³ is 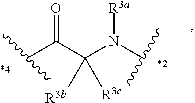
wherein:
R^{3a} is selected from H and C₁₋₆-alkyl;
R^{3b} is selected from H and C₁₋₆-alkyl;
R^{3c} is selected from C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)—(5-6 membered heterocycloalkyl), and —C₁₋₆-alkyl-N(H)—C₁₋₆-alkyl-(5-6 membered heterocycloalkyl), wherein the C₁₋₆-alkyl, —C₁₋₆-alkyl-C(O)NH—C₁₋₆-alkyl, —C₁₋₆-alkyl-phenyl, —C₁₋₆-alkyl-N(H)-phenyl, —C₁₋₆-alkyl-N(H)—C(O)-phenyl, —C₁₋₆-alkyl-N(H)—C₁₋₆-alkyl-phenyl, —C₁₋₆-alkyl-(5-6 membered heterocycloalkyl), —C₁₋₆-alkyl-N(H)-(5-6 membered heterocycloalkyl), —C₁₋₆-alkyl-N(H)—C(O)—(5-6 membered heterocycloalkyl), and —C₁₋₆-alkyl-N(H)—C₁₋₆-alkyl-(5-6 membered heterocycloalkyl) of R^{3c} are optionally substituted with 1, 2, 3, 4, 5, or 6 substituents independently selected from —CN, —C(O)OH, —C₁₋₆-alkyl-C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *4 indicates the point of attachment to A⁴ and *2 indicates the point of attachment to A²;
A⁴ is 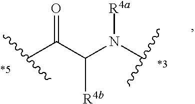
wherein:
R^{4a} is selected from H, C₁₋₆-alkyl, and —CH₂-phenyl, wherein the C₁₋₆-alkyl and —CH₂-phenyl of R^{4a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{4b} is selected from H, C₁₋₆-alkyl, —C₁₋₆-alkyl-phenyl, —C₁₋₆-alkyl-O-phenyl, —C₁₋₆-alkyl-(5-6 membered heteroaryl), —C₁₋₇-alkyl-C(O)OH, and —C₁₋₆-alkyl-NH—C(O)OH, wherein the C₁₋₆-alkyl, —C₁₋₆-alkyl-phenyl, —C₁₋₆-alkyl-O-phenyl, and —C₁₋₆-alkyl-(5-6 membered heteroaryl) of R^{4b} are optionally substituted with 1, 2, or 3 substituents independently selected from halo, —C(O)NH₂, —NH₂, —NHC₁₋₆-alkyl, —N(C₁₋₆-alkyl)₂, —N(H)—C(O)—C₁₋₆-alkyl, —N(H)—C(O)—NH₂, —N(H)—C(O)—N(H) (C₁₋₆-alkyl), —N(H)—C(O)—N(C₁₋₆-alkyl)₂, —OH, —OC₁₋₆-alkyl, and C₁₋₆-alkyl; and
wherein *5 indicates the point of attachment to A⁵ and *3 indicates the point of attachment to A³;
A⁵ is 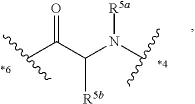
wherein:
R^{5a} is selected from H, C₁₋₆-alkyl, and —CH₂-phenyl, wherein the C₁₋₆-alkyl and —CH₂-phenyl of R^{5a} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5b} is selected from H, C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c}, wherein the C₁₋₆-alkyl, —C₁₋₆-alkyl-phenyl, —C₁₋₆-alkyl-(5-6 membered heteroaryl), —C₁₋₇-alkyl-C(O)R^{5c}, —C₁₋₆-alkyl-O—C(O)R^{5c}, and —C₁₋₆-alkyl-NH—C(O)R^{5c} of R^{5b} are each optionally substituted with 1, 2, or 3 substituents independently selected from halo, —NH₂, —NHC₁₋₆-alkyl, —N(C₁₋₆-alkyl)₂, —OH, —OC₁₋₆-alkyl, and C₁₋₆-alkyl;
R^{5c} is selected from H, C₁₋₆-alkyl, —NH₂, —NHC₁₋₆-alkyl, —N(C₁₋₆-alkyl)₂, —OH, —OC₁₋₆-alkyl, C₁-6-haloalkyl, C₁₋₆-alkyl-C(O)OH, and C₁₋₆-alkyl-C(O) OC₁₋₆-alkyl; and
wherein *6 indicates the point of attachment to A⁶ and *4 indicates the point of attachment to A⁴;
A⁶ is 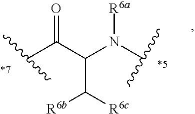
wherein:
R^{6a} is selected from H and C₁₋₆-alkyl;
R^{6b} is selected from H and C₁₋₆-alkyl;
R^{6c} is selected from C₁₋₆-alkyl and 5-10 membered heteroaryl, wherein the C₁₋₆-alkyl and 5-10 membered heteroaryl of R^{6c} are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —OC(O)—C₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *5 indicates the point of attachment to A⁵ and *7 indicates the point of attachment to A⁷;
A⁷ is 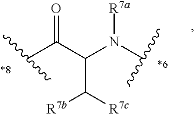
wherein:
R^{7a} is selected from H and C₁₋₆-alkyl;
R^{7b} is selected from H and C₁₋₆-alkyl;
R^{7c} is selected from C₁₋₆-alkyl and 5-10 membered heteroaryl, wherein the C₁₋₆-alkyl and 5-10 membered heteroaryl are each optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, —OC₁₋₆-alkyl, —N(H)—C(O)—C₁₋₆-alkyl, —N(H)—C(O)—NH₂, —N(H)—C(O)—N(H) (C₁₋₆-alkyl), and —N(H)—C(O)—N(C₁₋₆-alkyl)₂; and
wherein *8 indicates the point of attachment to A⁸ and *6 indicates the point of attachment to A⁶;
A⁸ is 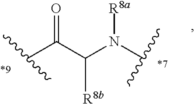
wherein:
R^{8a} is selected from H and C₁₋₆-alkyl;
R^{8b} is selected from H, C₁₋₆-alkyl, C₃₋₈-cycloalkyl, 5-7 membered heterocycloalkyl ring, and 5-10 membered heteroaryl, wherein the C₁₋₆-alkyl, C₃₋₈-cycloalkyl, 5-7 membered heterocycloalkyl ring, and 5-10 membered heteroaryl of R⁸% are optionally substituted with 1, 2, 3, or 4 substituents independently selected from C₁₋₆-alkyl, —CN, halo, —C(O)NH₂, —C(O)OH, —NH₂, —NHC₁₋₆-alkyl, —N(C₁₋₆-alkyl)₂, —OH, and —OC₁₋₆-alkyl; and
wherein *9 indicates the point of attachment to A⁹ and *7 indicates the point of attachment to A⁷;
A⁹ is 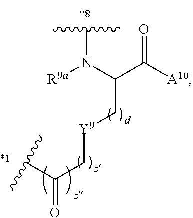
wherein:
Y⁹ is selected from a bond, C(O), NH, NC₁₋₆-alkyl, O, and S;
R^{9a} is selected from H and C₁₋₆-alkyl;
d is 1, 2, or 3; and
z′ and z″ are each independently 0 or 1; and
wherein *8 indicates the point of attachment to A⁸ and *1 indicates the point of attachment to A¹; and
A¹⁰ is 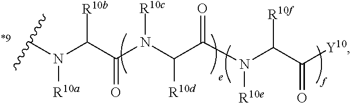
wherein:
Y¹⁰ is selected from OH and N(R^{10g})(R^{10h});
R^{10a}, R^{10c}, R^{10e}, R^{10g}, and R^{10h} are each independently selected from H and C₁₋₆-alkyl;
R^{10b} is selected from H, C₁₋₆-alkyl, —C₁₋₆-alkyl-phenyl, —C₁₋₆-alkyl-(5-6 membered heteroaryl), —C₁₋₇-alkyl-C(O)R¹⁰ⁱ, —C₁₋₆-alkyl-O—C(O)R¹⁰ⁱ, and —C₁₋₆-alkyl-NH—C(O)R¹⁰ⁱ, wherein the C₁₋₆-alkyl, —C₁₋₆-alkyl-phenyl, and —C₁₋₆-alkyl-(5-6 membered heteroaryl) of R^{10b} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10a} and R^{10b}, together with the atoms to which they are attached, form a 5-7 membered heterocycloalkyl ring;
R^{10d} is selected from H, C₁₋₆-alkyl, —C₁₋₆-alkyl-phenyl, —C₁₋₆-alkyl-(5-6 membered heteroaryl), —C₁₋₇-alkyl-C(O)R^{10j}, —C₁₋₆-alkyl-O—C(O)R^{10j}, and —C₁₋₆-alkyl-NH—C(O)R^{10j}, wherein the C₁₋₆-alkyl, —C₁₋₆-alkyl-phenyl, and —C₁₋₆-alkyl-(5-6 membered heteroaryl) of R^{10d} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10c} and R^{10d}, together with the atoms to which they are attached, form a 5-7 membered heterocycloalkyl ring;
R^{10f} is selected from H, C₁₋₆-alkyl, —C₁₋₆-alkyl-phenyl, —C₁₋₆-alkyl-(5-6 membered heteroaryl), —C₁₋₇-alkyl-C(O)R^{10k}, —C₁₋₆-alkyl-O—C(O)R^{10k}, and —C₁₋₆-alkyl-NH—C(O)R^{10k}, wherein the C₁₋₆-alkyl, —C₁₋₆-alkyl-phenyl, and —C₁₋₆-alkyl-(5-6 membered heteroaryl) of R^{10f} are optionally substituted with 1, 2, or 3 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, —C(O)OH, —C₁₋₆-alkyl-C(O)OH, and —C(O)NH₂;
alternatively, R^{10e} and R^{10f}, together with the atoms to which they are attached, form a 5-7 membered heterocycloalkyl ring;
R¹⁰ⁱ, R^{10j}, and R^{10k} are each independently selected from H, C₁₋₆-alkyl, C₃₋₇ cycloalkyl, 5-6 membered heteroaryl, and 3-7 membered heterocycloalkyl, wherein the C₃₋₇ cycloalkyl, 5-6 membered heteroaryl, and 3-7 membered heterocycloalkyl of R¹⁰ⁱ, R^{10j}, and R^{10k} are each optionally substituted with 1, 2, 3, or 4 substituents independently selected from halo, —NH₂, —NHC₁₋₆-alkyl, —N(C₁₋₆-alkyl)₂, —N₃, —OH, —OC₁₋₆-alkyl, C₁₋₆-alkyl, C₁₋₆-alkyl, —C(O)OH, —C(O)NH₂, and C₁₋₆-alkyl-C(O)NH₂;
e and f are each independently 0 or 1; and
wherein *9 indicates the point of attachment to A⁹.

74. (canceled)


SEQUENCE LISTING (121 sequences)

SEQ ID NO: 1 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXP

SEQ ID NO: 2 - protein, 10 aa, synthetic construct
  SITE 1: note = Acetyl-L-aspartic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-4-amino-2-(methylamino)butanoic acid
  SITE 10: note = Amidated (S)-3-(4-fluorophenyl)-2-(methylamino)propanoic acid
  SITE 1..9: note = Cyclized via the side chain
DYSXXXWXXX

SEQ ID NO: 3 - protein, 10 aa, synthetic construct
  SITE 1: note = Acetyl-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-4-amino-2-(methylamino)butanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 4 - protein, 10 aa, synthetic construct
  SITE 1: note = Acetyl-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-2,3-diaminopropanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 5 - protein, 10 aa, synthetic construct
  SITE 1: note = Acetyl-L-glutamic acid
  SITE 4: note = Methyl-L-homophenylalanine
  SITE 5: note = Methyl-L-serine
  SITE 6: note = (S)-2-amino-3-(pyrazolo[1,5-a]pyridin-3-yl)propanoi c acid
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 6 - protein, 10 aa, synthetic construct
  SITE 1: note = Acetyl-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 7 - protein, 11 aa, synthetic construct
  SITE 1: note = Acetyl-L-homoglutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXP

SEQ ID NO: 8 - protein, 10 aa, synthetic construct
  SITE 1: note = Acetyl-L-aspartic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
DYSXXXWXXX

SEQ ID NO: 9 - protein, 11 aa, synthetic construct
  SITE 1: note = Acetyl-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
EYSXXXWXXXP

SEQ ID NO: 10 - protein, 10 aa, synthetic construct
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 11 - protein, 10 aa, synthetic construct
  SITE 4: note = Methyl-L-homophenylalanine
  SITE 5: note = N2-methyl-N6-(2,2,2-trifluoroacetyl)-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 12 - protein, 10 aa, synthetic construct
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
EYSXXXWXXX

SEQ ID NO: 13 - protein, 10 aa, synthetic construct
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
  SITE 1..9: note = Cyclized via the side chain
DYSXXXWXXX

SEQ ID NO: 14 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 15 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 16 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 17 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXAXXXK

SEQ ID NO: 18 - protein, 11 aa, synthetic construct
  SITE 1: note = Acetyl-L-homoglutamic acid
  SITE 2: note = D-Tyrosine
  SITE 3: note = D-Serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 19 - protein, 10 aa, synthetic construct
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = Methyl-L-cysteine
  SITE 10: note = Amidated (S)-3-(4-fluorophenyl)-2-(methylamino)propanoic acid
  SITE 1..9: note = Intrachain disulfide bridge
CYSXXXWXXX

SEQ ID NO: 20 - protein, 10 aa, synthetic construct
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = Methyl-L-homocysteine
  SITE 10: note = Amidated (S)-3-(4-fluorophenyl)-2-(methylamino)propanoic acid
  SITE 1..9: note = Intrachain disulfide bridge
CYSXXXWXXX

SEQ ID NO: 21 - protein, 11 aa, synthetic construct
  SITE 1: note = (2-carboxyacetyl)-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 22 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 1..9: note = Cyclized via the side chain
XYSXXXWXXXK

SEQ ID NO: 23 - protein, 11 aa, synthetic construct
  SITE 1: note = Adipic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 24 - protein, 11 aa, synthetic construct
  SITE 1: note = (S)-2-(3-carboxypropanamido)hexanedioic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 25 - protein, 11 aa, synthetic construct
  SITE 1: note = Homoglutamic acid-2-(1H-tetrazol-5-yl)acetic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 26 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 27 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 28 - protein, 11 aa, synthetic construct
  SITE 1: note = Acetyl-L-homoglutamic acid
  SITE 4: note = Methyl-L-homophenylalanine
  SITE 5: note = N6-(3-hydroxypropanoyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 29 - protein, 12 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e
  SITE 12: note = Amidated D-Lysine
  SITE 1..9: note = Cyclized via the side chain
EYSXXXWXXXXK

SEQ ID NO: 30 - protein, 12 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e
  SITE 12: note = Amidated Residue
  SITE 1..9: note = Cyclized via the side chain
EYSXXXWXXXXK

SEQ ID NO: 31 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
EYSXXXWXXXK

SEQ ID NO: 32 - protein, 11 aa, synthetic construct
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated N6-(4-(carboxymethyl)piperazine-1-carbonyl)-L-lysine
  SITE 1..9: note = Cyclized via the side chain
EYSXXXWXXXX

SEQ ID NO: 33 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 7: note = D-Tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 34 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid
  SITE 1..9: note = Cyclized via the side chain
XYSXXXWXXXK

SEQ ID NO: 35 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid
  SITE 1..9: note = Cyclized via the side chain
XYSXXXAXXXK

SEQ ID NO: 36 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 37 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 38 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXAXXXK

SEQ ID NO: 39 - protein, 11 aa, synthetic construct
  SITE 1: note = Acetyl-L-homoglutamic acid
  SITE 2: note = D-Tyrosine
  SITE 3: note = D-Serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 40 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 41 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 42 - protein, 11 aa, synthetic construct
  SITE 1: note = Acetyl-L-homoglutamic acid
  SITE 4: note = Methyl-L-homophenylalanine
  SITE 5: note = N6-(3-hydroxypropanoyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 43 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 7: note = D-Tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 44 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2''-(1,4,7-triazonane-1,4,7-triyl)triacetic acid
  SITE 1..9: note = Cyclized via the side chain
XYSXXXWXXXK

SEQ ID NO: 45 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2''-(1,4,7-triazonane-1,4,7-triyl)triacetic acid
  SITE 1..9: note = Cyclized via the side chain
XYSXXXWXXXK

SEQ ID NO: 46 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to (R)-2-(4,7,10-tris(ca rboxymethyl)-1,4,7,10-tetraazacyclododecan-1-yl)pentanedio ic acid
  SITE 1..9: note = Cyclized via the side chain
XYSXXXWXXXK

SEQ ID NO: 47 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2-(4,7-bis(carboxymet hyl)-1,4,7-triazonan-1-yl)pentanedioic acid
  SITE 1..9: note = Cyclized via the side chain
XYSXXXWXXXK

SEQ ID NO: 48 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 49 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 50 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 51 - protein, 11 aa, synthetic construct
  SITE 1: note = (2-carboxyacetyl)-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 52 - protein, 11 aa, synthetic construct
  SITE 1: note = (S)-2-(3-carboxypropanamido)hexanedioic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 53 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Lysine conjugated to 2,2',2'',2'''-(1,4,7,10-tetraa zacyclododecane-1,4,7,10-tetrayl)tetraacetic acid and labelled with 175Lu
  SITE 1..9: note = Cyclized via the side chain
XYSXXXWXXXK

SEQ ID NO: 54 - protein, 11 aa, synthetic construct
  SITE 1: note = Homoglutamic acid-2-(1H-tetrazol-5-yl)acetic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 55 - protein, 11 aa, synthetic construct
  SITE 1: note = Adipic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 56 - protein, 11 aa, synthetic construct
  SITE 1: note = (S)-2-(2-hydroxyacetamido)hexanedioic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 57 - protein, 11 aa, synthetic construct
  SITE 1: note = N-acetyl-O-(carboxymethyl)-L-serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 58 - protein, 11 aa, synthetic construct
  SITE 1: note = Homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 59 - protein, 11 aa, synthetic construct
  SITE 1: note = Acetyl-L-homoglutamic acid
  SITE 4: note = Methyl-L-homophenylalanine
  SITE 5: note = N6-(2-carboxyacetyl)-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 60 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 61 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 62 - protein, 11 aa, synthetic construct
  SITE 1: note = Acetyl-L-homoglutamic acid
  SITE 4: note = Methyl-L-homophenylalanine
  SITE 5: note = N6-(3-hydroxypropanoyl)-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 63 - protein, 12 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e
  SITE 12: note = Amidated D-Lysine conjugated to 2,2',2'',2'''-(1,4, 7,10-tetraazacyclododecane-1,4,7,10-tetrayl)tetraacetic acid and labelled with 175Lu
  SITE 1..9: note = Cyclized via the side chain
EYSXXXWXXXXK

SEQ ID NO: 64 - protein, 12 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e
  SITE 12: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
EYSXXXWXXXXK

SEQ ID NO: 65 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
EYSXXXWXXXK

SEQ ID NO: 66 - protein, 11 aa, synthetic construct
  SITE 1: note = Glutamic acid conjugated to 2,2',2'',2'''-(1,4,7,10 -tetraazacyclododecane-1,4,7,10-tetrayl)tetraacetic acid and labelled with 175Lu AcOH salt
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated N6-(4-(carboxymethyl)piperazine-1-carbonyl)-L-lysine
  SITE 1..9: note = Cyclized via the side chain
EYSXXXWXXXX

SEQ ID NO: 67 - protein, 12 aa, synthetic construct
  SITE 1: note = Beta-alanine conjugated to 2,2',2'',2'''-(1,4,7,10- tetraazacyclododecane-1,4,7,10-tetrayl)tetraacetic acid and labelled with 175Lu AcOH salt
  SITE 5: note = Methyl-L-homophenylalanine
  SITE 6: note = N6-acetyl-N2-methyl-L-lysine
  SITE 7: note = 5-hydroxy tryptophan
  SITE 9: note = Allothreonine
  SITE 10: note = (S)-3-amino-2-(methylamino)propanoic acid
  SITE 11: note = (S)-3-(4-fluorophenyl)-2-(methylamino)propanoic acid
  SITE 12: note = Amidated N6-(4-(carboxymethyl)piperazine-1-carbonyl)-L-lysine
  SITE 1..9: note = Cyclized via the side chain
XEYSXXXWXXXX

SEQ ID NO: 68 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with natural Ga
  SITE 1..9: note = Cyclized via the side chain
XYSXXXWXXXK

SEQ ID NO: 69 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with natural La
  SITE 1..9: note = Cyclized via the side chain
XYSXXXWXXXK

SEQ ID NO: 70 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 7: note = D-Tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 71 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXAXXXK

SEQ ID NO: 72 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to (R)-2-(4,7,10-tris(ca rboxymethyl)-1,4,7,10-tetraazacyclododecan-1-yl)pentanedio ic acid labelled with natural Ga
  SITE 1..9: note = Cyclized via the side chain
XYSXXXWXXXK

SEQ ID NO: 73 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2-(4,7-bis(carboxymet hyl)-1,4,7-triazonan-1-yl)pentanedioic acid and labelled with natural Ga
  SITE 1..9: note = Cyclized via the side chain
XYSXXXWXXXK

SEQ ID NO: 74 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2-(4,7-bis(carboxymet hyl)-1,4,7-triazonan-1-yl)pentanedioic acid and labelled with natural Ga
  SITE 1..9: note = Cyclized via the side chain
XYSXXXWXXXK

SEQ ID NO: 75 - protein, 11 aa, synthetic construct
  SITE 1: note = Acetyl-L-homoglutamic acid
  SITE 2: note = D-Tyrosine
  SITE 3: note = D-Serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with natural La
  SITE 1..9: note = Cyclized via the side chain
XYSXXXWXXXK

SEQ ID NO: 76 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with natural La
  SITE 1..9: note = Cyclized via the side chain
XYSXXXWXXXK

SEQ ID NO: 77 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to SulfoCy5
  SITE 1..9: note = Cyclized via the side chain
XYSXXXWXXXK

SEQ ID NO: 78 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to SulfoCy5
  SITE 1..9: note = Cyclized via the side chain
XYSXXXAXXXK

SEQ ID NO: 79 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e attached to amidated Lysine conjugated to SulfoCy5 via 1-amino-3,6,9,12,15,18,21,24,27,30-decaoxatritriacontan-33 -oic acid
  SITE 1..9: note = Cyclized via the side chain
EYSXXXWXXXX

SEQ ID NO: 80 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to SulfoCy5
  SITE 1..9: note = Cyclized via the side chain
EYSXXXWXXXK

SEQ ID NO: 81 - protein, 12 aa, synthetic construct
  SITE 1: note = Beta-alanine labelled with SulfoCy5
  SITE 5: note = Methyl-L-homophenylalanine
  SITE 6: note = N6-acetyl-N2-methyl-L-lysine
  SITE 7: note = 5-hydroxy tryptophan
  SITE 9: note = Allothreonine
  SITE 10: note = (S)-3-amino-2-(methylamino)propanoic acid
  SITE 11: note = (S)-3-(4-fluorophenyl)-2-(methylamino)propanoic acid
  SITE 12: note = Amidated N6-(4-(carboxymethyl)piperazine-1-carbonyl)-L-lysine
  SITE 1..9: note = Cyclized via the side chain
XEYSXXXWXXXX

SEQ ID NO: 82 - protein, 11 aa, synthetic construct
  SITE 1: note = Homoglutamic acid-1-amino-3,6,9,12,15,18,21,24,27,3 0-decaoxatritriacontan-33-oic acid conjugated to SulfoCy5
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 83 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid conjugated to SulfoCy5
  SITE 1..9: note = Cyclized via the side chain
XYSXXXWXXXK

SEQ ID NO: 84 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid conjugated to biotin
  SITE 1..9: note = Cyclized via the side chain
EYSXXXWXXXK

SEQ ID NO: 85 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e attached to amidated lysine-biotin via 1-amino-3,6,9,12, 15,18,21,24,27,30-decaoxatritriacontan-33-oic acid
  SITE 1..9: note = Cyclized via the side chain
EYSXXXWXXXX

SEQ ID NO: 86 - protein, 12 aa, synthetic construct
  SITE 1: note = Beta-alanine-1-amino-3,6,9,12,15,18,21,24,27,30-dec aoxatritriacontan-33-oic acid conjugated to biotin
  SITE 5: note = Methyl-L-homophenylalanine
  SITE 6: note = N6-acetyl-N2-methyl-L-lysine
  SITE 7: note = 5-hydroxy tryptophan
  SITE 9: note = Allothreonine
  SITE 10: note = (S)-3-amino-2-(methylamino)propanoic acid
  SITE 11: note = (S)-3-(4-fluorophenyl)-2-(methylamino)propanoic acid
  SITE 12: note = Amidated N6-(4-(carboxymethyl)piperazine-1-carbonyl)-L-lysine
  SITE 1..9: note = Cyclized via the side chain
XEYSXXXWXXXX

SEQ ID NO: 87 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid conjugated to biotin
  SITE 1..9: note = Cyclized via the side chain
XYSXXXWXXXK

SEQ ID NO: 88 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid conjugated to biotin
  SITE 1..9: note = Cyclized via the side chain
XYSXXXAXXXK

SEQ ID NO: 89 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 6-fluoronicotinic acid
  SITE 1..9: note = Cyclized via the side chain
XYSXXXWXXXK

SEQ ID NO: 90 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 5-carboxy-N,N,N-trimethylpyridin-2-aminium salt
  SITE 1..9: note = Cyclized via the side chain
XYSXXXWXXXK

SEQ ID NO: 91 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 6-fluoronicotinic acid
  SITE 1..9: note = Cyclized via the side chain
XYSXXXWXXXK

SEQ ID NO: 92 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 5-carboxy-N,N,N-trimethylpyridin-2-aminium salt
  SITE 1..9: note = Cyclized via the side chain
XYSXXXWXXXK

SEQ ID NO: 93 - protein, 11 aa, synthetic construct
  SITE 1: note = Homoglutamic acid-1-amino-3,6,9,12,15,18,21,24,27,3 0,33,36,39,42,45,48,51,54,57,60,63,66,69,72-tetracosaoxape ntaheptacontane-75-peroxoic acid conjugated to docosane-1,11,11-tricarboxylic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 94 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 177Lu
  SITE 1..9: note = Cyclized via the side chain
XYSXXXWXXXK

SEQ ID NO: 95 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 177Lu
  SITE 1..9: note = Cyclized via the side chain
XYSXXXWXXXK

SEQ ID NO: 96 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Lysine conjugated to 2,2',2'',2'''-(1,4,7,10-tetraa zacyclododecane-1,4,7,10-tetrayl)tetraacetic acid and labelled with 177Lu
  SITE 1..9: note = Cyclized via the side chain
XYSXXXAXXXK

SEQ ID NO: 97 - protein, 11 aa, synthetic construct
  SITE 1: note = Acetyl-L-homoglutamic acid
  SITE 2: note = D-Tyrosine
  SITE 3: note = D-Serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Lysine conjugated to 2,2',2'',2'''-(1,4,7,10-tetraa zacyclododecane-1,4,7,10-tetrayl)tetraacetic acid and labelled with 177Lu
  SITE 1..9: note = Cyclized via the side chain
XYSXXXWXXXK

SEQ ID NO: 98 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 68Ga
  SITE 1..9: note = Cyclized via the side chain
XYSXXXWXXXK

SEQ ID NO: 99 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 68Ga
  SITE 1..9: note = Cyclized via the side chain
XYSXXXWXXXK

SEQ ID NO: 100 - protein, 11 aa, synthetic construct
  SITE 1: note = Homoglutamic acid-2,5,8,11,14,17,20,23,26,29,32,35- dodecaoxaoctatriacontan-38-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 225Ac
  SITE 1..9: note = Cyclized via the side chain
XYSXXXWXXXK

SEQ ID NO: 101 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 225Ac
  SITE 1..9: note = Cyclized via the side chain
XYSXXXWXXXK

SEQ ID NO: 102 - protein, 11 aa, synthetic construct
  SITE 1: note = Homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 103 - protein, 11 aa, synthetic construct
  SITE 1: note = N-acetyl-O-(carboxymethyl)-L-serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 104 - protein, 11 aa, synthetic construct
  SITE 1: note = (2-carboxyacetyl)-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 105 - protein, 11 aa, synthetic construct
  SITE 1: note = (S)-2-(3-carboxypropanamido)hexanedioic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 106 - protein, 11 aa, synthetic construct
  SITE 1: note = Homoglutamic acid-2-(1H-tetrazol-5-yl)acetic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 107 - protein, 11 aa, synthetic construct
  SITE 1: note = Adipic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 108 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
EYSXXXWXXXK

SEQ ID NO: 109 - protein, 11 aa, synthetic construct
  SITE 1: note = Acetyl-L-homoglutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid
  SITE 1..9: note = Cyclized via the side chain
XYSXXXWXXXK

SEQ ID NO: 110 - protein, 10 aa, synthetic construct
  SITE 1: note = Acetyl-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
EYSXXXWXXX

SEQ ID NO: 111 - protein, 10 aa, synthetic construct
  SITE 1: note = (((9H-fluoren-9-yl)methoxy)carbonyl)-L-glutamic acid
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = Amidated (S)-3-(4-fluorophenyl)-2-(methylamino)propanoic acid
EYSXXXWXXX

SEQ ID NO: 112 - protein, 11 aa, synthetic construct
  SITE 1: note = N-(((9H-fluoren-9-yl)methoxy)carbonyl)-O-(carboxyme thyl)-L-serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated N6-diazo-L-lysine
SYSXXXWXXXX

SEQ ID NO: 113 - protein, 10 aa, synthetic construct
  SITE 4: note = Methyl-L-homophenylalanine
  SITE 5: note = Methyl-L-serine
  SITE 6: note = 5-hydroxy tryptophan
  SITE 8: note = 2-cyclopropyl glycine
  SITE 9: note = Methyl-L-cysteine
  SITE 10: note = Amidated (S)-3-(4-fluorophenyl)-2-(methylamino)propanoic acid
CYSXXXWXXX

SEQ ID NO: 114 - protein, 11 aa, synthetic construct
  SITE 1: note = Acetyl-L-homoglutamic acid
  SITE 4: note = Methyl-L-homophenylalanine
  SITE 5: note = N6-(2-hydroxyacetyl)-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated N6-((allyloxy)carbonyl)-L-lysine
  SITE 1..9: note = Cyclized via the side chain
XYSXXXWXXXK

SEQ ID NO: 115 - protein, 11 aa, synthetic construct
  SITE 1: note = Homoglutamic acid-1-(9H-fluoren-9-yl)-3-oxo-2,7,10, 13,16,19,22,25,28,31,34-undecaoxa-4-azaheptatriacontan-37- oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
XYSXXXWXXXK

SEQ ID NO: 116 - protein, 11 aa, synthetic construct
  SITE 1: note = Homoglutamic acid-1-amino-3,6,9,12,15,18,21,24,27,3 0-decaoxatritriacontan-33-oic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 117 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine-1-amino-3,6,9,12,15,18,21,24,27,30- decaoxatritriacontan-33-oic acid conjugated to biotin
EYSXXXWXXXK

SEQ ID NO: 118 - protein, 11 aa, synthetic construct
  SITE 1: note = Acetyl-L-glutamic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = Allothreonine
  SITE 9: note = (S)-3-amino-2-(methylamino)propanoic acid
  SITE 10: note = (S)-3-(4-fluorophenyl)-2-(methylamino)propanoic acid
  SITE 11: note = N6-(4-(carboxymethyl)piperazine-1-carbonyl)-L-lysin e attached to amidated Lysine via 1-amino-3,6,9,12,15,18,2 1,24,27,30-decaoxatritriacontan-33-oic acid
  SITE 1..9: note = Cyclized via the side chain
EYSXXXWXXXX

SEQ ID NO: 119 - protein, 11 aa, synthetic construct
  SITE 1: note = Homoglutamic acid-1-amino-3,6,9,12,15,18,21,24,27,3 0,33,36,39,42,45,48,51,54,57,60,63,66,69,72-tetracosaoxape ntaheptacontane-75-peroxoic acid conjugated to docosane-1,11,11-tricarboxylic acid
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Residue
  SITE 1..9: note = Cyclized via the side chain
XYSXXXWXXXK

SEQ ID NO: 120 - protein, 11 aa, synthetic construct
  SITE 1: note = Acetyl-L-homoglutamic acid
  SITE 2: note = D-Tyrosine
  SITE 3: note = D-Serine
  SITE 4: note = Methyl-L-homophenylalanine
  SITE 5: note = N6-acetyl-N2-methyl-L-lysine
  SITE 6: note = 5-hydroxy tryptophan
  SITE 8: note = 2-cyclopropyl glycine
  SITE 9: note = (S)-3-amino-2-(methylamino)propanoic acid
  SITE 10: note = (S)-3-(4-fluorophenyl)-2-(methylamino)propanoic acid
  SITE 11: note = Amidated Lysine conjugated to 2,2',2'',2'''-(1,4,7, 10-tetraazacyclododecane-1,4,7,10-tetrayl)tetraacetic acid and labelled with 175Lu
  SITE 1..9: note = Cyclized via the side chain
XYSXXXWXXXK

SEQ ID NO: 121 - protein, 12 aa, synthetic construct
  SITE 1: note = Beta-alanine
  SITE 5: note = Methyl-L-homophenylalanine
  SITE 6: note = N6-acetyl-N2-methyl-L-lysine
  SITE 7: note = 5-hydroxy tryptophan
  SITE 9: note = Allothreonine
  SITE 10: note = (S)-3-amino-2-(methylamino)propanoic acid
  SITE 11: note = (S)-3-(4-fluorophenyl)-2-(methylamino)propanoic acid
  SITE 12: note = Amidated N6-(4-(carboxymethyl)piperazine-1-carbonyl)-L-lysine
  SITE 1..9: note = Cyclized via the side chain
XEYSXXXWXXXX